(19) United States
(12) Patent Application Publication
(10) Pub. No.: US 2026/0258014 A1
(43) Pub. Date: Sep. 3, 2026

(54) JAK2 Inhibitors And Their Use As Pharmaceuticals

(71) Applicant: Prelude Therapeutics Incorporated, Wilmington, DE (US)

(72) Inventors: Andrew Combs, Kennett Square, PA (US); Song Mei, Wilmington, DE (US); Xiaowei Wu, Wilmington, DE (US); John Rose, Newark, DE (US); Klare Lazor Bersch, Glen Mills, PA (US); Danielle Roth, Wilmington, DE (US)

(21) Appl. No.: 19/414,669

(22) Filed: Dec. 10, 2025

Related U.S. Application Data
(60) Provisional application No. 63/730,023, filed on Dec. 10, 2024.
(60) Provisional application No. 63/843,353, filed on Jul. 14, 2025.

(51) Int. Cl. C07D 401/14; A61K 31/501; A61K 31/5513; A61K 31/553; C07D 401/12; C07D 403/12; C07D 405/14; C07D 413/14; C07D 417/14; C07D 471/04; C07D 491/048; C07D 493/10; C07D 519/00

(52) U.S. Cl. CPC C07D 401/14; A61K 31/501; A61K 31/5513; A61K 31/553; C07D 401/12; C07D 403/12; C07D 405/14; C07D 413/14; C07D 417/14; C07D 471/04; C07D 491/048; C07D 493/10; C07D 519/00

(57) ABSTRACT

The disclosure is directed to compounds of Formula I

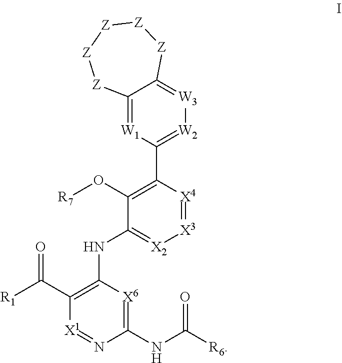

Pharmaceutical compositions comprising compounds of Formula I, as well as methods of their use and preparation, are also described.

This application claims the benefit of U.S. Provisional Application Nos. 63/730,023, filed Dec. 10, 2024, and 63/843,353, filed Jul. 14, 2025, the entireties of which are incorporated by reference herein.

### TECHNICAL FIELD

The disclosure is directed to JAK2 inhibitors and methods of their use.

### BACKGROUND

The Janus kinase (JAK) family of kinases (JAK1, JAK2, JAK3, and TYK2) are a family of intracellular, non-receptor tyrosine kinases that transduce cytokine-mediated signals. JAKs are in the cell selectively associated with the cytoplasmic domains of various cytokine receptors. Receptor-associated JAKs are activated in a ligand-dependent manner. Upon binding of the ligand and subsequent activation, JAKs can phosphorylate another JAK protein on the paired receptor and the intracellular tail of the receptors to which the JAKs are bound. These phosphorylated peptides serve as docking sites for a family of transcription factors, the signal transducers and activators of transcription (STAT). Upon binding of the STATs to the activated receptor-JAK complex, the STATs are phosphorylated, dimerize, and then are translocated to the nucleus where the binding of DNA and regulate gene expression occurs. Alterations in JAK2 signaling can occur through point mutations/deletions/insertions or chromosomal translocations. These JAK2 alterations drive diseases that are primarily characterized by abnormal proliferation of terminally differentiated myeloid cells. Examples of disease with JAK2 alterations are essential thrombocytosis or essential thrombocythemia (ET), polycythemia vera (PV), myelofibrosis (MF), primary myelofibrosis (PMF), and secondary myelofibrosis (SMF). Clinical features of these diseases include progressive anemia, splenomegaly, and constitutional symptoms (cough, fatigue, pruritus, and bone pain).

Existing compounds that inhibit JAK2 (e.g. ruxoltinib and fedratinib) have been developed for these myeloproliferative indications and have proven beneficial to patients in terms of spleen volume reduction and symptomatic improvement. However, a significant limitation in the clinical effectiveness of existing JAK2 inhibitors has been the inability to achieve clinically effective doses while avoiding toxicity in patients with myeloid proliferative disease, due to their strong inhibition of wildtype JAK2. Treatment of patients with the approved JAK2 inhibitors can result in anemia and thrombocytopenia and these toxicities are consistent with the known function of wildtype JAK2 in regulation of erythrocytes and platelets. Thus, there exists a need for JAK2 compounds that inhibit mutant JAK2 activity while sparing cytokine-mediated activity of wildtype JAK2, in order to increase the therapeutic window and allow for more complete inhibition of the mutant protein.

Additional small molecule JAK2 selective inhibitors are needed.

### SUMMARY OF THE INVENTION

The disclosure is directed to compounds of Formula I:

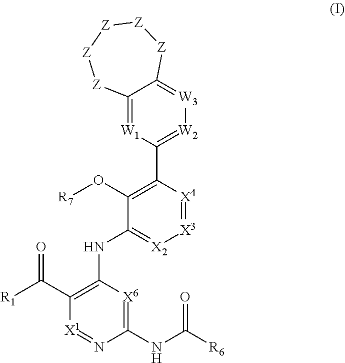

or a pharmaceutically acceptable salt or solvate or N-oxide thereof, wherein
each of W₁, W₂, and W₃ is independently N or CR₈;
each Z is independently O, S, SO₂, NR₄, NR₉ or C(R₈)₂, wherein at least 3 Z are C(R₈)₂ and wherein at least one Z is NR₄;
each R₈ is independently H, D, halogen, —OH, —CN, —NO₂, —C₁-C₆alkyl, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R_{b}R^{b}, —P(O)(OR^{b})(OR^{b}), —B(OR^{c})(OR^{d}) or —S(O)₂R^{b};
or any two R₈ groups attached to the same carbon form ═O;
or any two R₈ groups attached to the same carbon form a 3-6 membered spirocycloalkyl or spiroheterocycloalkyl ring;
or any two R₈ groups attached to adjacent carbon atoms form a 3-6 membered cycloalkyl or heterocycloalkyl ring;
each R₄ is independently -L¹-L²-L³;
each R₉ is independently H, —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl, —C(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR), —B(OR^{c})(OR^{d}) or —S(O)₂R^{b}; wherein each of —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl can be optionally substituted by 1-6 R_{f} groups;
each L¹ is independently absent, or is independently —(CR^{c}R^{d})ₚ—, —(CR^{a}R^{c})ₚO—, —S(O)—, —S(O)₂—, —C(═O)—, —C(═O)O—, —C(═O)NR^{a}—, —S(═O)NR^{a}—, —S(═O)₂NR^{a}—, or aryl, heteroaryl, cycloalkyl, cycloalkenyl, heterocycloalkyl, or heterocycloalkenyl;
each L² is independently absent, or is independently aryl, heteroaryl, cycloalkyl, cycloalkenyl, heterocycloalkyl, or heterocycloalkenyl;
each L³ is independently H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
p is 1, 2, 3 or 4;
each X₁, X₂, X₃, X₄, and X₆ is independently N or CR₁₀;
each R₁₀ is independently H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
R₁ is H, D, —C₁-C₆alkyl, —C₁-C₆haloalkyl, C₁-C₈ alkoxide, —C₂-C₆alkenyl, —C₂-C₆alkynyl, —NHR₅;
R₅ is H, —C₁-C₆alkyl, C₁-C₈ alkoxide, —C₂-C₆alkenyl, —C₂-C₆alkynyl, cycloalkyl, cycloalkenyl, heterocycloalkyl, or heterocycloalkenyl;
R₆ is H, D, —OH, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl or spiroheterocycloalkenyl;
R₇ is H, —C₁-C₆alkyl, —C₁-C₆haloalkyl, —C₂-C₆alkenyl, or cycloalkyl;
each R^{a} is independently H, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group;
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁-R₁₀ and L¹-L³ is optionally substituted by 1-6 R^{f} groups; and
each R^{f} is independently H, D, oxo, halogen, C₁-C₈ alkoxy, C₁-C₈ alkyl, C₁₋₆ haloalkyl, C₁₋₆ haloalkoxy, —OH, —CN, —NO₂, —C₂₋₆ alkenyl, —C₂₋₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

Stereoisomers of the compounds of Formula I, and the pharmaceutical salts and stereoisomers thereof, are also contemplated, described, and encompassed herein. Methods of using compounds of Formula I are described, as well as pharmaceutical compositions including the compounds of Formula I.

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any subcombination.

At various places in the present specification, substituents of compounds of the invention are disclosed in groups or in ranges. It is specifically intended that the invention include each and every individual subcombination of the members of such groups and ranges. For example, the term “C₁-C₆ alkyl” is specifically intended to individually disclose methyl, ethyl, C₃ alkyl, C₄ alkyl, C₅ alkyl, and C₆ alkyl. “C₀ alkyl” refers to a covalent bond.

It is further intended that the compounds of the invention are stable. As used herein “stable” refers to a compound that is sufficiently robust to survive isolation to a useful degree of purity from a reaction mixture, and preferably capable of formulation into an efficacious therapeutic agent.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable sub-combination.

The term “alkyl,” when used alone or as part of a substituent group, refers to a straight- or branched-chain hydrocarbon group having from 1 to 12 carbon atoms (“C₁-C₁₂”), preferably 1 to 6 carbons atoms (“C₁-C₆”), in the group. Examples of alkyl groups include methyl (Me, C₁alkyl), ethyl (Et, C₂alkyl), n-propyl (C₃alkyl), isopropyl (C₃alkyl), butyl (C₄alkyl), isobutyl (C₄alkyl), sec-butyl (C₄alkyl), tert-butyl (C₄alkyl), pentyl (C₅alkyl), isopentyl (C₅alkyl), tert-pentyl (C₅alkyl), hexyl (C₆alkyl), isohexyl (C₆alkyl), and the like. Alkyl groups may be optionally substituted.

Unless otherwise specified, in those embodiments wherein the alkyl group is substituted, the alkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the alkyl group is optionally substituted by 1-6 R groups selected from H, D, halogen, —OH, —CN, —OR^{a}, —SR^{a}, —NR^{a}R^{d}, or NR^{c}R^{d}; or the alkyl group is optionally substituted by 1-6 R groups.

The term “halo” or halogen refers to chloro, fluoro, bromo, or iodo.

The term “cycloalkyl” when used alone or as part of a substituent group refers to cyclic-containing, non-aromatic hydrocarbon groups having from 3 to 10 carbon atoms (“C₃-C₁₀”), preferably from 3 to 6 carbon atoms (“C₃-C₆”). Cycloalkyl groups of the disclosure include monocyclic groups, as well as multicyclic groups such as bicyclic and tricyclic groups. In those embodiments having at least one multicyclic cycloalkyl group, the cyclic groups can share one common atom (i.e., spirocyclic). In other embodiments having at least one multicyclic cycloalkyl group, the cyclic groups share two common atoms (e.g., fused or bridged). Examples of cycloalkyl groups include, for example, cyclopropyl (C₃), cyclobutyl (C₄), cyclopropylmethyl (C₄), cyclopentyl (C₅), cyclohexyl (C₆), 1-methylcyclopropyl (C₄), 2-methylcyclopentyl (C₄), adamantanyl (C₁₀), spiro[3.3]heptanyl, bicyclo[3.3.0]octanyl, and the like. Cycloalkyl groups of the disclosure are optionally substituted. Unless otherwise specified, in those embodiments wherein the cycloalkyl group is substituted, the cycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkyl group is optionally substituted by 1-6 R groups selected from H, D, halogen, —OH, —CN, —OR^{a}, —SR^{a}, —NR^{a}R^{d}, or NR^{c}R^{d}; or the cycloalkyl group is optionally substituted by 1-6 R^{f} groups.

The term “cycloalkenyl” when used alone or as part of a substituent group refers to monocyclic or multicyclic, partially saturated ring structure having from 3 to 10 carbon atoms (“C₃-C₁₀”), preferably from 3 to 6 carbon atoms (“C₃-C₆”). Cycloalkenyl groups of the disclosure include monocyclic groups, as well as multicyclic groups such as bicyclic and tricyclic groups. In those embodiments having at least one multicyclic cycloalkenyl group, the cyclic groups can share one common atom (i.e., spirocyclic). In other embodiments having at least one multicyclic cycloalkenyl group, the cyclic groups share two common atoms (e.g., fused or bridged). The term —C₃-C₆ cycloalkenyl refers to a cycloalkenyl group having between three and six carbon atoms. The cycloalkenyl group may be attached at any carbon atom of the partially saturated ring such that the result is a stable structure. Cycloalkenyl groups include groups in which the partially saturated ring is fused to an aryl group. Examples of cycloalkenyl groups include, for example, cyclopropenyl (C₃), cyclobutenyl (C₄), cyclopropenylmethyl (C₄), cyclopentenyl (C₅), cyclohexenyl (C₆), 1-methylcyclopropenyl (C₄), 2-methylcyclopentenyl (C₄), adamantenyl (C₁₀), spiro[3.3]heptenyl, bicyclo[3.3.0]octenyl, indanyl, and the like. Cycloalkenyl groups of the disclosure are optionally substituted. Unless otherwise specified, in those embodiments wherein the cycloalkenyl group is substituted, the cycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the cycloalkenyl group is optionally substituted by 1-6 R groups selected from H, D, halogen, —OH, —CN, —OR^{a}, —SR^{a}, —NR^{a}R^{d}, or NR^{c}R^{d}; or the cycloalkenyl group is optionally substituted by 1-6 R^{f} groups.

The term “heterocycloalkyl” when used alone or as part of a substituent group refers to any three to twelve membered monocyclic or multicyclic, saturated ring structure containing at least one heteroatom selected from the group consisting of O, N and S. Heterocycloalkyl groups of the disclosure include monocyclic groups, as well as multicyclic groups such as bicyclic and tricyclic groups. In those embodiments having at least one multicyclic heterocycloalkyl group, the cyclic groups can share one common atom (i.e., spirocyclic). In other embodiments having at least one multicyclic heterocycloalkyl group, the cyclic groups share two common atoms (e.g., fused or bridged). The term —C₃-C₆ heterocycloalkyl refers to a heterocycloalkyl group having between three and six carbon ring atoms. The heterocycloalkyl group may be attached at any heteroatom or carbon atom of the group such that the result is a stable structure. Examples of heterocycloalkyl groups include, but are not limited to, azepanyl, aziridinyl, azetidinyl, pyrrolidinyl, dioxolanyl, imidazolidinyl, pyrazolidinyl, piperazinyl, piperidinyl, dioxanyl, morpholinyl, dithianyl, thiomorpholinyl, oxazepanyl, oxiranyl, oxetanyl, quinuclidinyl, tetrahydrofuranyl, tetrahydropyranyl, piperazinyl, azepanyl, diazepanyl, oxepanyl, dioxepanyl, azocanyl diazocanyl, oxocanyl, dioxocanyl, azaspiro[2.2]pentanyl, oxaazaspiro[3.3]heptanyl, oxaspiro[3.3]heptanyl, dioxaspiro[3.3]heptanyl, 3-azabicyclo[3.1.0]hexanyl,

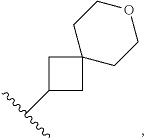 [SMILES: *C1CC2(CCOCC2)C1]

and the like. Heteroycloalkyl groups of the disclosure are optionally substituted. Unless otherwise specified, in those embodiments wherein the heterocycloalkyl group is substituted, the heterocycloalkyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkyl group is optionally substituted by 1-6 R groups selected from H, D, halogen, —OH, —CN, —OR^{a}, —SR^{a}, —NR^{a}R^{d}, or NR^{c}R^{d}; or the heterocycloalkyl group is optionally substituted by 1-6 R^{f} groups.

The term “heterocycloalkenyl” when used alone or as part of a substituent group refers to any three to twelve membered monocyclic or multicyclic, partially saturated ring structure containing at least one heteroatom selected from the group consisting of O, N and S. Heterocycloalkenyl groups of the disclosure include monocyclic groups, as well as multicyclic groups such as bicyclic and tricyclic groups. In those embodiments having at least one multicyclic heterocycloalkyenyl group, the cyclic groups can share one common atom (i.e., spirocyclic). In other embodiments having at least one multicyclic heterocycloalkenyl group, the cyclic groups share two common atoms (e.g., fused or bridged). The term —C₃-C₆ heterocycloalkenyl refers to a heterocycloalkenyl group having between three and six carbon atoms. The heterocycloalkenyl group may be attached at any heteroatom or carbon atom of ring system such that the result is a stable structure. Heterocycloalkenyl groups include groups in which the partially saturated ring is fused to an aryl group, such as, for example isoindoline,

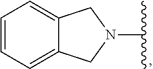 [SMILES: *N1Cc2ccccc2C1]

or for example a dihydroisoquinolinone such as 3,4-dihydroisoquinolin-1(2H)-one,

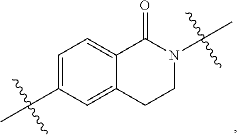 [SMILES: *c1ccc2c(c1)CCN(*)C2=O]

or in which the partially saturated ring is fused to a heteroaryl group, such as, for example, 6,7-dihydro-5H-pyrrolo[3,4-b]pyridine,

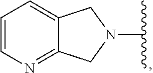

wherein [symbol] represents a point of attachment. Heteroycloalkenyl groups of the disclosure are optionally substituted. Unless otherwise specified, in those embodiments wherein the heterocycloalkenyl group is substituted, the heterocycloalkenyl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heterocycloalkenyl group is optionally substituted by 1-6 R groups selected from H, D, halogen, —OH, —CN, —OR^{a}, —SR^{a}, —NR^{a}R^{d}, or NR^{c}R^{d}; or the heterocycloalkenyl group is optionally substituted by 1-6 R^{f} groups.

The term “heterocyclic group,” when used alone or as part of a substituent group, refers to a heterocycloalkyl group or a heterocycloalkenyl group.

The term “heteroaryl” when used alone or as part of a substituent group refers to a mono- or bicyclic-aromatic ring structure including carbon atoms as well as up to five heteroatoms selected from nitrogen, oxygen, and sulfur. Heteroaryl rings can include a total of 5, 6, 7, 8, 9, or 10 ring atoms. Examples of heteroaryl groups include but are not limited to, pyrrolyl, furyl, thiophenyl (thienyl), oxazolyl, imidazolyl, pyrazolyl, isoxazolyl, isothiazolyl, triazolyl, thiadiazolyl, pyrazolyl, pyridinyl, pyridazinyl, pyrimidinyl, pyrazinyl, pyranyl, furazanyl, indolizinyl, indolyl, and the like. Heteroaryl groups of the disclosure are optionally substituted. Unless otherwise specified, in those embodiments wherein the heteroaryl group is substituted, the heteroaryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the heteroaryl group is optionally substituted by 1-6 R groups selected from H, D, halogen, —OH, —CN, —OR^{a}, —SR^{a}, —NR^{a}R^{d}, or NR^{c}R^{d}; or the heteroaryl group is optionally substituted by 1-6 R^{f} groups.

The term “aryl” when used alone or as part of a substituent group refers to a mono- or bicyclic-aromatic carbon ring structure. Aryl rings can include a total of 5, 6, 7, 8, 9, or 10 ring atoms. Examples of aryl groups include but are not limited to, phenyl, napthyl, and the like. Aryl groups of the disclosure are optionally substituted. Unless otherwise specified, in those embodiments wherein the aryl group is substituted, the aryl group can be substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the aryl group is optionally substituted by 1-6 R groups selected from H, D, halogen, —OH, —CN, —OR^{a}, —SR^{a}, —NR^{a}R^{d}, or NR^{c}R^{d}; or the aryl group is optionally substituted by 1-6 R^{f} groups.

The term “spirocycloalkyl” when used alone or as part of a substituent group refers to a cycloalkyl ring that shares one carbon atom with another cycloalkyl ring.

The term “spirocycloalkenyl” when used alone or as part of a substituent group refers to a cycloalkenyl ring that shares one carbon atom with another cycloalkyl or cycloalkenyl ring.

The term “spiroheterocycloalkyl” when used alone or as part of a substituent group refers to a heterocycloalkyl ring that shares one carbon atom with another cycloalkyl or heterocycloalkyl ring.

The term “spiroheterocycloalkenyl” when used alone or as part of a substituent group refers to a heterocycloalkenyl ring that shares one carbon atom with another cycloalkyl, heterocycloalkyl, or heterocycloalkenyl ring.

When a range of carbon atoms is used herein, for example, C₁-C₆, all ranges, as well as individual numbers of carbon atoms are encompassed, for example, “C₁₋₃” includes C₁₋₃, C₁₋₂, C₂₋₃, C₁, C₂, and C₃. The term “C₁₋₆alk” refers to an aliphatic linker having 1, 2, 3, 4, 5, or 6 carbon atoms and includes, for example, —CH₂—, —CH(CH₃)—, —CH(CH₃)—CH₂—, and —C(CH₃)₂—. The term “—C₀alk-” refers to a bond.

The term “C₀-C₆alk” when used alone or as part of a substituent group refers to an aliphatic linker having 0, 1, 2, 3, 4, 5 or 6 carbon atoms. The term “—C₁alk-”, for example, refers to a —CH₂—.

The term “—C₀alk-” refers to a bond.

Unless otherwise specified, in those embodiments wherein the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are substituted, they can be optionally substituted with 1, 2, or 3 substituents independently selected from —OH, —CN, amino, halo, C₁-C₆alkyl, C₁-C₆alkoxy, C₁-C₆haloalkyl, and C₁-C₆haloalkoxy, —C(O)NH(C₁-C₆alkyl), —C(O)N(C₁-C₆alkyl)₂, —OC(O)NH(C₁-C₆alkyl), —OC(O)N(C₁-C₆alkyl)₂, —S(O)₂NH(C₁-C₆alkyl), and —S(O)₂N(C₁-C₆alkyl)₂. In other embodiments, the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are optionally substituted by 1-6 R groups selected from H, D, halogen, —OH, —CN, —OR^{a}, —SR^{a}, —NR^{a}R^{d}, or NR^{c}R^{d}; or the —C₁-C₆alkyl, —C₁-C₁₀ alkyl, —C₁-C₈ alkoxide, —C₂-C₆alkenyl, —C₂-C₁₀alkenyl, —C₂-C₆alkynyl, —C₂-C₁₀alkynyl, aryl, heteroaryl, cycloalkyl, cycloalkenyl, heterocycloalkenyl, and heterocycloalkyl groups are optionally substituted by 1-6 R^{f} groups.

As used herein, “alkoxy” refers to an —O-alkyl group. Example alkoxy groups include methoxy, ethoxy, propoxy (e.g., n-propoxy and isopropoxy), t-butoxy, and the like.

As used herein, “hydroxylalkyl” refers to an alkyl group substituted by OH.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Geometric isomers of olefins, C═N double bonds, and the like can also be present in the compounds described herein, and all such stable isomers are contemplated in the present invention. Geometric isomers of the compounds of the present invention are described and may be isolated as a mixture of isomers or as separated isomeric forms.

Compounds of the invention may also include tautomeric forms. All tautomeric forms are encompassed.

In some embodiments, the compounds of the present invention may exist as rotational isomers. In some embodiments, the compounds of the present invention exist as mixtures of rotational isomers in any proportion. In other embodiments, the compounds of the present invention exist as particular rotational isomers, substantially free of other rotational isomers.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 1 (1977) p. 1-19, each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols.

A “solvate” refers to a physical association of a compound of Formula I with one or more solvent molecules.

“Subject” includes humans. The terms “human,” “patient,” and “subject” are used interchangeably herein.

“Treating” or “treatment” of any disease or disorder refers, in one embodiment, to ameliorating the disease or disorder (i.e., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

“Compounds of the present disclosure,” and equivalent expressions, are meant to embrace compounds of Formula I as described herein, as well as its subgenera, which expression includes the stereoisomers (e.g., enantiomers, diastereomers) and constitutional isomers (e.g., tautomers) of compounds of Formula I as well as the pharmaceutically acceptable salts, where the context so permits.

As used herein, the term “isotopic variant” refers to a compound that contains proportions of isotopes at one or more of the atoms that constitute such compound that is greater than natural abundance. For example, an “isotopic variant” of a compound can be radiolabeled, that is, contain one or more radioactive isotopes, or can be labeled with non-radioactive isotopes such as for example, deuterium (²H or D), carbon-13 (¹³C), nitrogen-15 (¹⁵N), or the like. It will be understood that, in a compound where such isotopic substitution is made, the following atoms, where present, may vary, so that for example, any hydrogen may be ²H/D, any carbon may be ¹³C, or any nitrogen may be ¹⁵N, and that the presence and placement of such atoms may be determined within the skill of the art.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers,” for example, diastereomers, enantiomers, and atropisomers. The compounds of this disclosure may possess one or more asymmetric centers; such compounds can therefore be produced as individual (R)- or (S)-stereoisomers at each asymmetric center, or as mixtures thereof. Unless indicated otherwise, the description or naming of a particular compound in the specification and claims is intended to include all stereoisomers and mixtures, racemic or otherwise, thereof. Where one chiral center exists in a structure, but no specific stereochemistry is shown for that center, both enantiomers, individually or as a mixture of enantiomers, are encompassed by that structure. Where more than one chiral center exists in a structure, but no specific stereochemistry is shown for the centers, all enantiomers and diastereomers, individually or as a mixture, are encompassed by that structure. The methods for the determination of stereochemistry and the separation of stereoisomers are well-known in the art.

The disclosure is directed to compounds of Formula I:

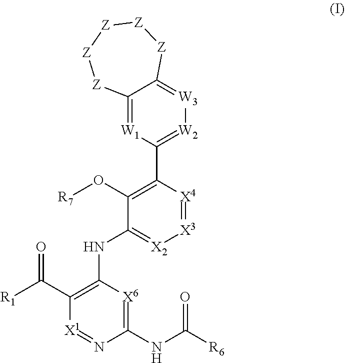

or a pharmaceutically acceptable salt or solvate or N-oxide thereof, wherein
each of W₁, W₂, and W₃ is independently N or CR₈;
each Z is independently O, S, SO₂, NR₄, NR₉ or C(R₈)₂, wherein at least 3 Z are C(R₈)₂ and wherein at least one Z is NR₄;
each R₈ is independently H, D, halogen, —OH, —CN, —NO₂, —C₁-C₆alkyl, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or any two R₈ groups attached to the same carbon form ═O;
or any two R₈ groups attached to the same carbon form a 3-6 membered spirocycloalkyl or spiroheterocycloalkyl ring;
or any two R₈ groups attached to adjacent carbon atoms form a 3-6 membered cycloalkyl or heterocycloalkyl ring;
each R₄ is independently -L¹-L²-L³;
each R₉ is independently H, —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl, —C(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR), —B(OR^{c})(OR^{d}) or —S(O)₂R^{b}; wherein each of —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl can be optionally substituted by 1-6 R^{f} groups;
each L¹ is independently absent, or is independently —(CR^{c}R^{d})ₚ—, —(CR^{a}R^{c})ₚO—, —S(O)—, —S(O)₂—, —C(═O)—, —C(═O)O—, —C(═O)NR^{a}—, —S(═O)NR^{a}—, —S(═O)₂NR^{a}—, or aryl, heteroaryl, cycloalkyl, cycloalkenyl, heterocycloalkyl, or heterocycloalkenyl;
each L² is independently absent, or is independently aryl, heteroaryl, cycloalkyl, cycloalkenyl, heterocycloalkyl, or heterocycloalkenyl;
each L³ is independently absent or is independently H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R_{b}R^{b}, —P(O)(OR^{b})(OR^{b}), —B(OR^{c})(OR^{d}) or —S(O)₂R^{b};
p is 1, 2, 3 or 4;
each X₁, X₂, X₃, X₄, and X₆ is independently N or CR₁₀;
each R₁₀ is independently H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
R¹ is H, D, —C₁-C₆alkyl, —C₁-C₆haloalkyl, C₁-C₈ alkoxide, —C₂-C₆alkenyl, —C₂-C₆alkynyl, —NHR₅;
R₅ is H, —C₁-C₆alkyl, C₁-C₈ alkoxide, —C₂-C₆alkenyl, —C₂-C₆alkynyl, cycloalkyl, cycloalkenyl, heterocycloalkyl, or heterocycloalkenyl;
R₆ is H, D, —OH, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl or spiroheterocycloalkenyl;
R₇ is H, —C₁-C₆alkyl, —C₁-C₆haloalkyl, —C₂-C₆alkenyl, or cycloalkyl;
each R^{a} is independently H, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group;
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁-R₁₀ and L¹-L³ is optionally substituted by 1-6 R^{f} groups; and
each R^{f} is independently H, D, oxo, halogen, C₁-C₈ alkoxy, C₁-C₈ alkyl, C₁₋₆ haloalkyl, C₁₋₆ haloalkoxy, —OH, —CN, —NO₂, —C₂₋₆ alkenyl, —C₂₋₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R_{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d}, or NR^{c}R^{d}.

In some embodiments, each W₁, W₂, and W₃ in Formula (I) is independently N or CR₈.

In some embodiments, W₁ in Formula (I) is N. In other embodiments, W₁ in Formula (I) is CR₈.

In some embodiments, W₂ in Formula (I) is N. In other embodiments, W₂ in Formula (I) is CR₈.

In other embodiments, W₃ in Formula (I) is N. In other embodiments, W₃ in Formula (I) is CR₈.

In yet other embodiments, at least one of W₁, W₂, and W₃ is CR₈. In yet other embodiments, each W₁, W₂, and W₃ is CR₈.

In some embodiments, each Z in Formula (I) is independently O, S, SO₂, NR₄, NR₉ or C(R₈)₂, wherein at least 3 Z are C(R₈)₂ and wherein at least one Z is NR₄.

In some embodiments, three Z are C(R₈)₂. In other embodiments, four Z are C(R₈)₂. In other embodiments, each Z is C(R₈)₂.

In some embodiments, one Z in Formula (I) is O and four Z are C(R₈)₂. In some embodiments, one Z in Formula (I) is O, one Z is NR₄ and three Z are C(R₈)₂. In other embodiments, two Z in Formula (I) are O and three Z are C(R₈)₂.

In some embodiments, one Z in Formula (I) is S and four Z are C(R₈)₂. In some embodiments, one Z in Formula (I) is S, one Z is NR₄ and three Z are C(R₈)₂. In other embodiments, two Z in Formula (I) are S and three Z are C(R₈)₂.

In some embodiments, one Z in Formula (I) is SO₂ and four Z are C(R₈)₂. In some embodiments, one Z in Formula (I) is SO₂, one Z is NR₄ and three Z are C(R₈)₂. In other embodiments, two Z in Formula (I) are SO₂ and three Z are C(R₈)₂.

In some embodiments, one Z in Formula (I) is NR₄ and four Z are C(R₈)₂. In some embodiments, one Z in Formula (I) is NR₄, one Z is NR₉ and three Z are C(R₈)₂. In other embodiments, two Z in Formula (I) are SO₂ and three Z are C(R₈)₂.

In some embodiments, R₈ in Formula (I) is H, D, halogen, —OH, —CN, —NO₂, —C₁-C₆ alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R_{b}R^{b}, —P(O)(OR^{b})(OR^{b}), —B(OR^{C})(OR^{d}) or —S(O)₂R^{b}.

In some embodiments, R₈ in Formula I is H. In some embodiments, R₈ in Formula I is D. In some embodiments, R₈ in Formula I is halogen. In some embodiments, R₈ in Formula I is —OH. In some embodiments, R₈ in Formula I is —CN. In some embodiments, R₈ in Formula I is —NO₂. In some embodiments, R₈ in Formula I is C₁-C₆ alkyl. In some embodiments, R₈ in Formula I is C₁-C₈ alkoxide. In some embodiments, R₈ in Formula I is —C₂-C₆ alkenyl. In some embodiments, R₈ in Formula I is —C₂-C₆ alkynyl. In some embodiments, R₈ in Formula I is aryl. In some embodiments, R₈ in Formula I is heteroaryl. In other embodiments, R₈ in Formula I is cycloalkyl. In other embodiments, R₈ in Formula I is cycloalkenyl. In other embodiments, R₈ in Formula I is heterocycloalkyl. In other embodiments, R₈ in Formula I is heterocycloalkyl. In other embodiments, R₈ in Formula I is OR^{a}. In other embodiments, R₈ in Formula I is SR^{a}. In other embodiments, R₈ in Formula I is —NR^{c}R^{d}. In other embodiments, R₈ in Formula I is —NR^{a}R^{c}. In other embodiments, R₈ in Formula I is —C(O)R^{b}. In other embodiments, R₈ in Formula I is —OC(O)R^{b}. In other embodiments, R₈ in Formula I is —C(O)OR^{b}. In yet other embodiments, R₈ in Formula I is —C(O)NR^{c}R^{d}. In yet other embodiments, R₈ in Formula I is —S(O)R^{b}. In yet other embodiments, R₈ in Formula I is —S(O)₂NR^{c}R^{d}. In yet other embodiments, R₈ in Formula I is —S(O)(═NR^{b})R^{b}. In yet other embodiments, R₈ in Formula I is —SF₅. In yet other embodiments, R₈ in Formula I is —P(O)R^{b}R^{b}. In yet other embodiments, R₈ in Formula I is —P(O)(OR^{b})(OR^{b}). In yet other embodiments, R₈ in Formula I is —B(OR^{c})(OR^{d}). In yet other embodiments, R₈ in Formula I is —S(O)₂R^{b}.

In yet other embodiments, any two R₈ groups attached to the same carbon form ═O. In yet other embodiments, any two R₈ groups attached to the same carbon form a 3-6 membered spirocycloalkyl or spiroheterocycloalkyl ring. In yet other embodiments, any two R₈ groups attached to adjacent carbon atoms form a 3-6 membered cycloalkyl or heterocycloalkyl ring.

In some embodiments, each R₄ in Formula (I) is independently -L¹-L²-L³.

In some embodiments, each R₉ in Formula (I) is independently H, D, —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl, —C(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}.

In some embodiments, R₉ in Formula I is H. In some embodiments, R₉ in Formula I is D. In some embodiments, R₉ in Formula I is C₁-C₆ alkyl. In some embodiments, R₉ in Formula I is —C₂-C₆ alkenyl. In some embodiments, R₉ in Formula I is —C₂-C₆ alkynyl. In some embodiments, R₉ in Formula I is aryl. In some embodiments, R₉ in Formula I is heteroaryl. In other embodiments, R₉ in Formula I is cycloalkyl. In other embodiments, R₉ in Formula I is cycloalkenyl. In other embodiments, R₉ in Formula I is heterocycloalkyl. In other embodiments, R₉ in Formula I is heterocycloalkyl. In other embodiments, R₉ in Formula I is heterocycloalkenyl. In other embodiments, R₉ in Formula I is spirocycloalkyl. In other embodiments, R₉ in Formula I is spiroheterocycloalkyl. In other embodiments, R₉ in Formula I is spirocycloalkenyl. In other embodiments, R₉ in Formula I is spiroheterocycloalkenyl. In other embodiments, R₉ in Formula I is —C(O)R^{b}. In other embodiments, R₉ in Formula I is —C(O)OR^{b}. In yet other embodiments, R₉ in Formula I is —C(O)NR^{c}R^{d}. In yet other embodiments, R₉ in Formula I is —S(O)R^{b}. In yet other embodiments, R₉ in Formula I is —S(O)₂NR^{c}R^{d}. In yet other embodiments, R₉ in Formula I is —S(O)(═NR^{b})R^{b}. In yet other embodiments, R₉ in Formula I is —SF₅. In yet other embodiments, R₉ in Formula I is —P(O)R^{b}R^{b}. In yet other embodiments, R₉ in Formula I is —P(O)(OR^{b})(OR^{b}). In yet other embodiments, R₉ in Formula I is —B(OR^{c})(OR^{d}). In yet other embodiments, R₉ in Formula I is —S(O)₂R^{b}.

In some embodiments, each of —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl in R₉ can be optionally substituted by 1-6 R^{f} groups.

In some embodiments, each L¹ in Formula (I) is independently absent, or is independently —(CR^{c}R^{d})ₚ—, —(CR^{a}R^{c})ₚO—, —O—, —S—, —S(O)—, —S(O)₂—, —C(═O)—, —NR^{a}—, —OC(═O)—, —C(═O)O—, —NR^{a}C(O)—, —C(═O)NR^{a}—, —OC(═O)N(R^{a})—, —NR^{a}C(O)O—, —S(═O)NR^{a}—, —NR^{a}S(O)—, —S(═O)₂NR^{a}—, or —NR^{a}S(O)₂—, aryl, heteroaryl, cycloalkyl, cycloalkenyl, heterocycloalkyl, or heterocycloalkenyl.

In some embodiments, L¹ in Formula (I) is absent. In some embodiments, L¹ in Formula (I) is —(CR^{c}R^{d})ₚ—. In some embodiments, L¹ in Formula (I) is —(CR^{a}R^{c})ₚO—. In some embodiments, L¹ in Formula (I) is —O—. In some embodiments, L¹ in Formula (I) is —S—. In some embodiments, L¹ in Formula (I) is —S(O)—. In some embodiments, L¹ in Formula (I) is —S(O)₂—. In some embodiments, L¹ in Formula (I) is —C(═O)—. In some embodiments, L¹ in Formula (I) is —NR^{a}—. In other embodiments, L¹ in Formula (I) is —OC(═O)—. In other embodiments, L¹ in Formula (I) is —C(═O)O—. In other embodiments, L¹ in Formula (I) is —NR^{a}C(O)—. In other embodiments, L¹ in Formula (I) is —C(═O)NR^{a}—. In other embodiments, L¹ in Formula (I) is —OC(═O)N(R^{a})—. In other embodiments, L¹ in Formula (I) is —NR^{a}C(O)O—. In other embodiments, L¹ in Formula (I) is —S(═O)NR^{a}—. In other embodiments, L¹ in Formula (I) is —NR^{a}S(O)—. In yet other embodiments, L¹ in Formula (I) is —S(═O)₂NR^{a}—. In yet other embodiments, L¹ in Formula (I) is —NR^{a}S(O)₂—. In yet other embodiments, L¹ in Formula (I) is aryl. In yet other embodiments, L¹ in Formula (I) is heteroaryl. In yet other embodiments, L¹ in Formula (I) is cycloalkyl. In yet other embodiments, L¹ in Formula (I) is cycloalkenyl. In yet other embodiments, L¹ in Formula (I) is heterocycloalkyl. In yet other embodiments, L¹ in Formula (I) is heterocycloalkenyl.

In some embodiments, L¹ in Formula (I) is —(CR^{c}R^{d})ₚ—. In some embodiments, L¹ in Formula (I) is —CH₂—.

In some embodiments, p in Formula (I) is 1, 2, 3 or 4. In some embodiments, p in Formula (I) is 0. In some embodiments, p in Formula (I) is 1. In some embodiments, p in Formula (I) is 2. In other embodiments, p in Formula (I) is 3. In other embodiments, p in Formula (I) is 4.

In some embodiments, each L² in Formula (I) is independently absent, or is independently aryl, heteroaryl, cycloalkyl, cycloalkenyl, heterocycloalkyl, or heterocycloalkenyl.

In some embodiments, L² in Formula (I) is absent. In some embodiments, L² in Formula (I) is aryl. In some embodiments, L² in Formula (I) is heteroaryl. In other embodiments, L² in Formula (I) is cycloalkyl. In other embodiments, L² in Formula (I) is cycloalkenyl. In yet other embodiments, L² in Formula (I) is heterocycloalkyl. In yet other embodiments, L² in Formula (I) is heterocycloalkenyl.

In some embodiments, L² in Formula (I) is phenyl or pyridyl. In some embodiments, L² in Formula (I) is phenyl. In other embodiments, L² in Formula (I) is pyridyl.

In some embodiments, L³ in Formula (I) is independently absent, or is independently H, D, halogen, —OH, —CN, —NO₂, —C₁-C₆ alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R_{b}R^{b}, —P(O)(OR^{b})(OR^{b}), —B(OR^{c})(OR^{d}) or —S(O)₂R^{b}.

In some embodiments, L³ in Formula (I) is absent. In some embodiments, L³ in Formula I is H. In some embodiments, L³ in Formula I is D. In some embodiments, L³ in Formula I is halogen. In some embodiments, L³ in Formula I is —OH. In some embodiments, L³ in Formula I is —CN. In some embodiments, L³ in Formula I is —NO₂. In some embodiments, L³ in Formula I is C₁-C₆ alkyl. In some embodiments, L³ in Formula I is C₁-C₈ alkoxide. In some embodiments, L³ in Formula I is —C₂-C₆ alkenyl. In some embodiments, L³ in Formula I is —C₂-C₆ alkynyl. In some embodiments, L³ in Formula I is aryl. In some embodiments, L³ in Formula I is heteroaryl. In other embodiments, L³ in Formula I is cycloalkyl. In other embodiments, L³ in Formula I is cycloalkenyl. In other embodiments, L³ in Formula I is heterocycloalkyl. In other embodiments, L³ in Formula I is heterocycloalkyl. In other embodiments, L³ in Formula I is OR^{a}. In other embodiments, L³ in Formula I is SR^{a}. In other embodiments, L³ in Formula I is —NR^{c}R^{d}. In other embodiments, L³ in Formula I is —NR^{a}R^{c}. In other embodiments, L³ in Formula I is —C(O)R^{b}. In other embodiments, L³ in Formula I is —OC(O)R^{b}. In other embodiments, L³ in Formula I is —C(O)OR^{b}. In yet other embodiments, L³ in Formula I is —C(O)NR^{c}R^{d}. In yet other embodiments, L³ in Formula I is —S(O)R^{b}. In yet other embodiments, L³ in Formula I is —S(O)₂NR^{c}R^{d}. In yet other embodiments, L³ in Formula I is —S(O)(═NR^{b})R^{b}. In yet other embodiments, L³ in Formula I is —SF₅. In yet other embodiments, L³ in Formula I is —P(O)R_{b}R^{b}. In yet other embodiments, L³ in Formula I is —P(O)(OR^{b})(OR^{b}). In yet other embodiments, L³ in Formula I is —B(OR^{c})(OR^{d}). In yet other embodiments, L³ in Formula I is —S(O)₂R^{b}.

In some embodiments, L³ in Formula (I) is H or —C₁-C₆alkyl. In some embodiments, L³ in Formula (I) is H or CH₃. In some embodiments, L³ in Formula (I) is H. In some embodiments, L³ in Formula (I) is CH₃.

In some embodiments, L³ in Formula (I) is —C(O)NR^{c}R^{d} wherein R^{c} and R^{d} are each independently —C₁-C₁₀ alkyl; or R^{c} and R^{d}, together with the atom to which they are both attached, form a monocyclic or multicyclic heterocycloalkyl.

In some embodiments, L³ in Formula (I) is —C(O)NR^{c}R^{d} wherein R^{c} and R^{d} are each independently —C₁-C₁₀ alkyl. In other embodiments, L³ in Formula (I) is —C(O)NR^{c}R^{d} wherein R^{c} and R^{d} are each independently —C₁-C₁₀ alkyl; or R^{c} and R^{d}, together with the atom to which they are both attached, form a monocyclic or multicyclic heterocycloalkyl.

In some embodiments, L³ in Formula (I) is —C(O)NR^{c}R^{d} and at least one of R^{c} and R^{d} is methyl.

In some embodiments, each X₁, X₂, X₃, X₄, and X₆ in Formula (I) is independently N or CR₁₀.

In some embodiments, X₁ in Formula (I) is N. In some embodiments, X₁ in Formula (I) is CR₁₀. In other embodiments, X₁ in Formula (I) is CH.

In some embodiments, X₂ in Formula (I) is N. In other embodiments, X₂ in Formula (I) is CR₁₀.

In some embodiments, X₃ in Formula (I) is N. In other embodiments, X₃ in Formula (I) is CR₁₀.

In some embodiments, X₄ in Formula (I) is N. In other embodiments, X₄ in Formula (I) is CR₁₀.

In some embodiments, X₂ in Formula (I) is N and X₄ in Formula (I) is N.

In other embodiments, X₆ in Formula (I) is N. In other embodiments, X₆ in Formula (I) CR₁₀.

In some embodiments, each of X₂, X₃, and X₄ in Formula (I) is CH. In some embodiments, X₆ in Formula (I) is CH.

In some embodiments, R₁₀ in Formula (I) is H, D, halogen, —OH, —CN, —NO₂, —C₁-C₆ alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, —OR^{a}, —SR^{a}, —NR^{c}R^{d}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{C})(OR^{d}) or —S(O)₂R^{b}.

In some embodiments, R₁₀ in Formula I is H. In some embodiments, R₁₀ in Formula I is D. In some embodiments, R₁₀ in Formula I is halogen. In some embodiments, R₁₀ in Formula I is —OH. In some embodiments, R₁₀ in Formula I is —CN. In some embodiments, R₁₀ in Formula I is —NO₂. In some embodiments, R₁₀ in Formula I is C₁-C₆ alkyl. In some embodiments, R₁₀ in Formula I is C₁-C₈ alkoxide. In some embodiments, R₁₀ in Formula I is —C₂-C₆ alkenyl. In some embodiments, R₁₀ in Formula I is —C₂-C₆ alkynyl. In some embodiments, R₁₀ in Formula I is aryl. In some embodiments, R₁₀ in Formula I is heteroaryl. In other embodiments, R₁₀ in Formula I is cycloalkyl. In other embodiments, R₁₀ in Formula I is cycloalkenyl. In other embodiments, R₁₀ in Formula I is heterocycloalkyl. In other embodiments, R₁₀ in Formula I is heterocycloalkyl. In other embodiments, R₁₀ in Formula I is OR^{a}. In other embodiments, R₁₀ in Formula I is SR^{a}. In other embodiments, R₁₀ in Formula I is —NR^{c}R^{d}. In other embodiments, R₁₀ in Formula I is —C(O)R^{b}. In other embodiments, R₁₀ in Formula I is —OC(O)R^{b}. In other embodiments, R₁₀ in Formula I is —C(O)OR^{b}. In yet other embodiments, R₁₀ in Formula I is —C(O)NR^{c}R^{d}. In yet other embodiments, R₁₀ in Formula I is —S(O)R^{b}. In yet other embodiments, R₁₀ in Formula I is —S(O)₂NR^{c}R^{d}. In yet other embodiments, R₁₀ in Formula I is —S(O)(═NR^{b})R^{b}. In yet other embodiments, R₁₀ in Formula I is —SF₅. In yet other embodiments, R₁₀ in Formula I is —P(O)R_{b}R^{b}. In yet other embodiments, R₁₀ in Formula I is —P(O)(OR^{b})(OR^{b}). In yet other embodiments, R₁₀ in Formula I is —B(OR^{c})(OR^{d}). In yet other embodiments, R₁₀ in Formula I is —S(O)₂R^{b}.

In some embodiments, R₁ in Formula (I) is H, D, —C₁-C₆alkyl, —C₁-C₆haloalkyl, C₁-C₈ alkoxide, —C₂-C₆alkenyl, —C₂-C₆alkynyl, —NHR₅.

In some embodiments, R₁ in Formula I is H. In some embodiments, R₁ in Formula I is D. In some embodiments, R₁ in Formula I is C₁-C₆ alkyl. In some embodiments, R₁ in Formula I is C₁-C₈ haloalkyl. In other embodiments, R₁ in Formula I is C₁-C₈ alkoxide. In other embodiments, R₁ in Formula I is —C₂-C₆ alkenyl. In other embodiments, R₁ in Formula I is —C₂-C₆ alkynyl.

In some embodiments, R₁ is methyl optionally substituted by 1-6 D atoms. In other embodiments, R₁ is methyl substituted by at least one D atom.

In some embodiments, R¹ in Formula I is —NHR₅. In some embodiments, R₅ in Formula (I) is H, —C₁-C₆alkyl, C₁-C₈ alkoxide, —C₂-C₆alkenyl, —C₂-C₆alkynyl, cycloalkyl, cycloalkenyl, heterocycloalkyl, or heterocycloalkenyl.

In some embodiments, R₅ in Formula I is H. In some embodiments, R₅ in Formula I is C₁-C₆ alkyl. In some embodiments, R₅ in Formula I is C₁-C₈ alkoxide. In some embodiments, R₅ in Formula I is —C₂-C₆ alkenyl. In some embodiments, R₅ in Formula I is —C₂-C₆ alkynyl. In some embodiments, R₅ in Formula I is aryl. In some embodiments, R₅ in Formula I is heteroaryl. In other embodiments, R₅ in Formula I is cycloalkyl. In other embodiments, R₅ in Formula I is cycloalkenyl. In other embodiments, R₅ in Formula I is heterocycloalkyl. In other embodiments, R₅ in Formula I is heterocycloalkenyl.

In some embodiments, R₅ is methyl optionally substituted by 1-6 D atoms. In some embodiments, R₅ is methyl substituted by at least one D atom. In some embodiments, R₅ in Formula I is methyl or CD₃. In some embodiments, R₅ in Formula I is CD₃.

In some embodiments, R₆ in Formula (I) is H, D, —OH, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl or spiroheterocycloalkenyl.

In some embodiments, R₆ in Formula I is H. In some embodiments, R₆ in Formula I is D. In some embodiments, R₆ in Formula I is —OH. In some embodiments, R₆ in Formula I is C₁-C₆ alkyl. In some embodiments, R₆ in Formula I is C₁-C₈ alkoxide. In some embodiments, R₆ in Formula I is —C₂-C₆ alkenyl. In some embodiments, R₆ in Formula I is —C₂-C₆ alkynyl. In some embodiments, R₆ in Formula I is aryl. In some embodiments, R₆ in Formula I is heteroaryl. In other embodiments, R₆ in Formula I is cycloalkyl. In other embodiments, R₆ in Formula I is cycloalkenyl. In other embodiments, R₆ in Formula I is heterocycloalkyl. In other embodiments, R₆ in Formula I is heterocycloalkenyl. In yet other embodiments, R₆ in Formula I is spirocycloalkyl. In yet other embodiments, R₆ in Formula I is spiroheterocycloalkyl. In yet other embodiments, R₆ in Formula I is spiroheterocycloalkenyl.

In some embodiments, R₆ in Formula (I) is —C₁-C₆alkyl, cycloalkyl or spirocycloalkyl, wherein the —C₁-C₆alkyl, cycloalkyl and spirocycloalkyl are optionally substituted by 1-6 R_{f} groups. In some embodiments, R₆ in Formula (I) is —C₁-C₆alkyl optionally substituted by 1-6 R_{f} groups. In other embodiments, R₆ in Formula (I) is cycloalkyl optionally substituted by 1-6 R_{f} groups. In other embodiments, R₆ in Formula (I) is spirocycloalkyl optionally substituted by 1-6 R^{f} groups.

In some embodiments, R₆ in Formula (I) is cyclopropyl optionally substituted by 1-6 R_{f} groups. In some embodiments, R₆ in Formula (I) is fluorine substituted cyclopropane.

In some embodiments, R₆ in Formula (I) is —C₁-C₆alkyl optionally substituted by 1-6 R_{f} groups.

In some embodiments, R₇ in Formula (I) is H, D, —C₁-C₆ alkyl, —C₁-C₆ haloalkyl, —C₂-C₆alkenyl, or cycloalkyl.

In some embodiments, R₇ in Formula I is H. In some embodiments, R₇ in Formula I is D. In some embodiments, R₇ in Formula I is —C₁-C₆ alkyl. In some embodiments, R₇ in Formula I is —C₁-C₆ haloalkyl. In some embodiments, R₇ in Formula I is —C₂-C₆ alkenyl. In other embodiments, R₇ in Formula I is cycloalkyl.

In some embodiments, each R^{a} in Formula I is independently H, D, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl.

In some embodiments, R^{a} in Formula I is H. In some embodiments, R^{a} in Formula I is D. In some embodiments, R^{a} in Formula I is —C₁-C₁₀alkyl. In some embodiments, R^{a} in Formula I is —C₂-C₁₀ alkenyl. In some embodiments, R^{a} in Formula I is —C₂-C₁₀ alkynyl. In other embodiments, R^{a} in Formula I is aryl. In other embodiments, R^{a} in Formula I is cycloalkyl. In other embodiments, R^{a} in Formula I is cycloalkenyl. In yet other embodiments, R^{a} in Formula I is heteroaryl. In yet other embodiments, R^{a} in Formula I is heterocycloalkyl. In yet other embodiments, R^{a} in Formula I is heterocycloalkenyl.

In some embodiments, each R^{b} in Formula I is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl.

In some embodiments, R^{b} in Formula I is H. In some embodiments, R^{b} in Formula I is D. In some embodiments, R^{b} in Formula I is —C₁-C₆ alkyl. In some embodiments, R^{b} in Formula I is —C₂-C₆ alkenyl. In some embodiments, R^{b} in Formula I is —C₂-C₆ alkynyl. In other embodiments, R^{b} in Formula I is aryl. In other embodiments, R^{b} in Formula I is cycloalkyl. In other embodiments, R^{b} in Formula I is cycloalkenyl. In other embodiments, R^{b} in Formula I is heteroaryl. In other embodiments, R^{b} in Formula I is heterocycloalkyl. In other embodiments, R^{b} in Formula I is heterocycloalkenyl.

In some embodiments, each R^{c} or R^{d} in Formula I is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl.

In some embodiments, R^{c} or R^{d} in Formula I is H. In some embodiments, R^{c} or R^{d} in Formula I is D. In some embodiments, R^{c} or R^{d} in Formula I is —C₁-C₁₀ alkyl. In some embodiments, R^{c} or R^{d} in Formula I is —C₂-C₆ alkenyl. In some embodiments, R^{c} or R^{d} in Formula I is —C₂-C₆ alkynyl. In other embodiments, R^{c} or R^{d} in Formula I is —OC₁-C₆alkyl. In other embodiments, R^{c} or R^{d} in Formula I is —O-cycloalkyl. In other embodiments, R^{c} or R^{d} in Formula I is aryl. In other embodiments, R^{c} or R^{d} in Formula I is cycloalkyl. In other embodiments, R^{c} or R^{d} in Formula I is cycloalkenyl. In other embodiments, R^{c} or R^{d} in Formula I is heteroaryl. In other embodiments, R^{c} or R^{d} in Formula I is heterocycloalkyl. In other embodiments, R^{c} or R^{d} in Formula I is heterocycloalkenyl.

In yet other embodiments, R^{c} and R^{d} in Formula I, together with the atom to which they are both attached, form a monocyclic or multicyclic heterocycloalkyl, or a monocyclic or multicyclic heterocyclo-alkenyl group. In yet other embodiments, R^{c} and R^{d} in Formula I form a monocyclic heterocycloalkyl. In yet other embodiments, R^{c} and R^{d} in Formula I form a multicyclic heterocycloalkyl. In yet other embodiments, R^{c} and R^{d} in Formula I form a monocyclic heterocyclo-alkenyl group. In yet other embodiments, R^{c} and R^{d} in Formula I form a multicyclic heterocyclo-alkenyl group.

In some embodiments, each R in Formula I, when present, is independently H, D, oxo, halogen, C₁-C₈ alkoxide, C₁-C₈ alkyl, haloalkyl, —OH, —CN, —NO₂, —C₂-C₆ alkenyl, —C₂-C₆ alkynyl, aryl, heteroaryl, cycloalkyl, cycloalkenyl, heterocycloalkyl, heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R_{b}R^{b}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 R groups selected from H, D, halogen, —OH, —CN, —OR^{a}, —SR^{a}, —NR^{a}R^{d}, or NR^{c}R^{d}.

In some embodiments, Rin Formula I is H. In some embodiments, Rin Formula I is D. In some embodiments, Rin Formula I is oxo. In some embodiments, R in Formula I is halogen. In some embodiments, R in Formula I is C₁-C₈ alkoxide. In some embodiments, R in Formula I is C₁-C₈ alkyl. In some embodiments, the C₁-C₈ alkyl is optionally substituted by 1-6 R groups selected from H, D, halogen, —OH, —CN, —OR^{a}, —SR^{a}, —NR^{a}R^{d}, or NR^{c}R^{d}. In some embodiments, R in Formula I is haloalkyl. In some embodiments, R^{f} in Formula I is —OH. In some embodiments, R^{f} in Formula I is —CN. In some embodiments, R^{f} in Formula I is —NO2. In some embodiments, R^{f} in Formula I is —C₂-C₆ alkenyl. In some embodiments, R^{f} in Formula I is —C₂-C₆ alkynyl. In some embodiments, R^{f} in Formula I is aryl. In some embodiments, R^{f} in Formula I is heteroaryl. In some embodiments, R^{f} in Formula I is cycloalkyl. In other embodiments, R^{f} in Formula I is cycloalkenyl. In other embodiments, R^{f} in Formula I is heterocycloalkyl. In other embodiments, R^{f} in Formula I is heterocycloalkenyl. In other embodiments, R^{f} in Formula I is —OR^{a}. In other embodiments, R^{f} in Formula I is —SR^{a}. In other embodiments, R^{f} in Formula I is —NR^{c}R^{d}. In other embodiments, R^{f} in Formula I is —NR^{a}R^{c}. In other embodiments, R^{f} in Formula I is —C(O)R^{b}. In other embodiments, R^{f} in Formula I is —OC(O)R^{b}. In other embodiments, R^{f} in Formula I is —C(O)OR^{b}. In other embodiments, R^{f} in Formula I is —C(O)NR^{c}R^{d}. In yet other embodiments, R^{f} in Formula I is —S(O)R^{b}. In yet other embodiments, R^{f} in Formula I is —S(O)₂NR^{c}R^{d}. In yet other embodiments, R^{f} in Formula I is —S(O)(═NR^{b})R. In yet other embodiments, R^{f} in Formula I is —SF₅. In yet other embodiments, R^{f} in Formula I is —P(O)R^{b}R^{b}. In yet other embodiments, R^{f} in Formula I is —P(O)(OR^{b})(OR^{b}). In yet other embodiments, R^{f} in Formula I is —B(OR^{c})(OR^{d}). In yet other embodiments, R^{f} in Formula I is —S(O)₂R^{b}. In yet other embodiments, R^{f} in Formula I is —C(O)NR^{b}OR^{b}. In yet other embodiments, R^{f} in Formula I is —S(O)₂OR^{b}. In yet other embodiments, R^{f} in Formula I is —OS(O)₂OR^{b}. In yet other embodiments, R^{f} in Formula I is —OPO(OR^{b})(OR).

In some embodiments, the compound of Formula (I) is a compound of Formula (II):

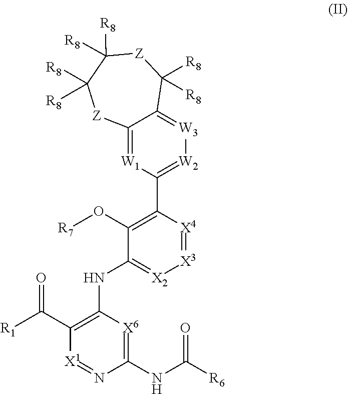

or a pharmaceutically acceptable salt or solvate or N-oxide thereof; wherein R₁, R₆, R₇, R₈, W₁, W₂, W₃, X₁, X₂, X₃, X₄, X₆, and Z are as defined herein.

In some embodiments, the compound of Formula (I) is a compound of Formula (III), Formula (IV) or Formula (V):

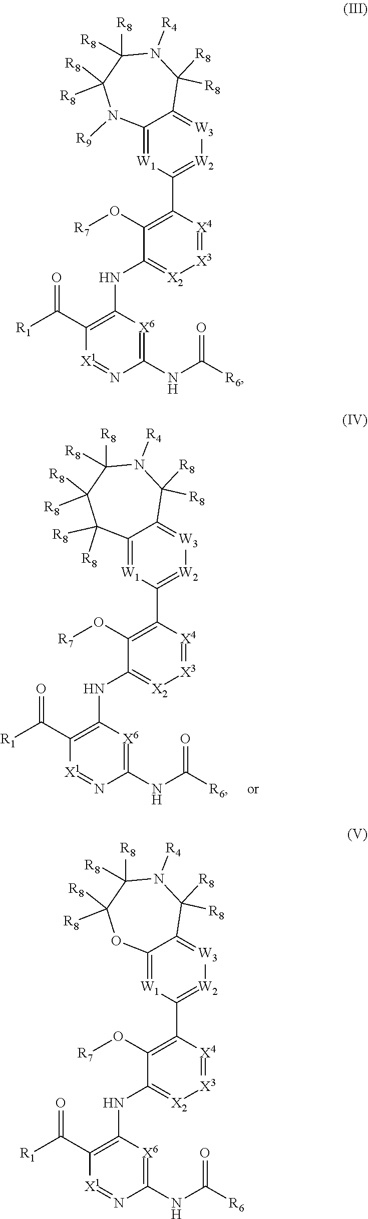

or a pharmaceutically acceptable salt or solvate or N-oxide thereof; wherein R₁, R₄, R₆, R₇, R₈, R₉, W₁, W₂, W₃, X₁, X₂, X₃, X₄, and X₆ are as defined herein.

In some embodiments, the compound of Formula (I) is a compound of Formula (III):

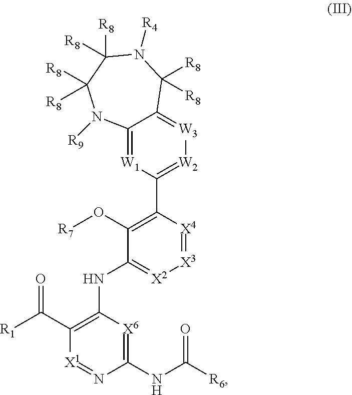

or a pharmaceutically acceptable salt or solvate or N-oxide thereof; wherein R₁, R₄, R₆, R₇, R₈, R₉, W₁, W₂, W₃, X₁, X₂, X₃, X₄, and X₆ are as defined herein.

In other embodiments, the compound of Formula (I) is a compound of Formula (IV):

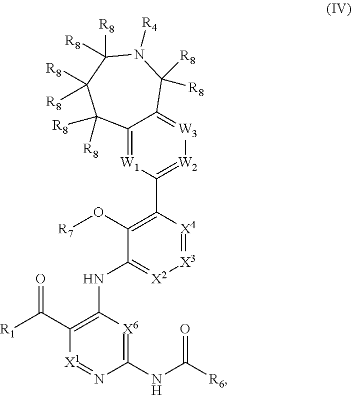

or a pharmaceutically acceptable salt or solvate or N-oxide thereof; wherein R₁, R₄, R₆, R₇, R₈, W₁, W₂, W₃, X₁, X₂, X₃, X₄, and X₆ are as defined herein.

In yet other embodiments, the compound of Formula (I) is a compound of Formula (V):

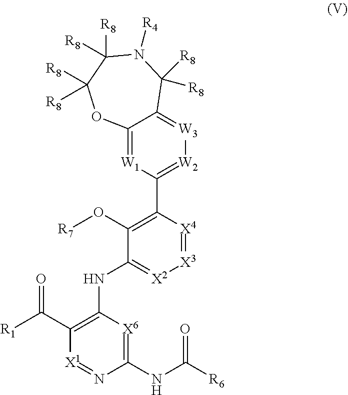

or a pharmaceutically acceptable salt or solvate or N-oxide thereof; wherein R₁, R₄, R₆, R₇, R₈, W₁, W₂, W₃, X₁, X₂, X₃, X₄, and X₆ are as defined herein.

In some embodiments, the compound of Formula (I) is a compound of Formula (VI), Formula (VII), Formula (VIII), Formula (IX), Formula (X), Formula (XI), Formula (XII), Formula (XIII), Formula (XIV), or Formula (XV):

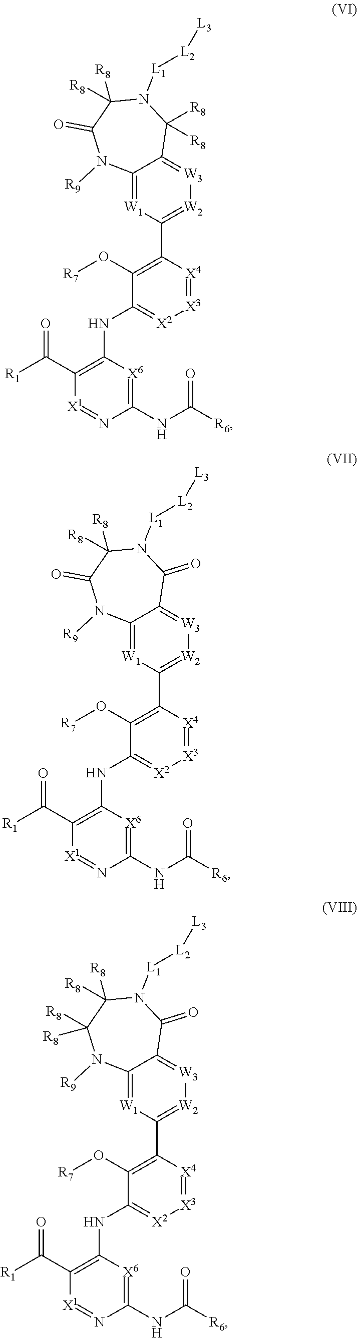

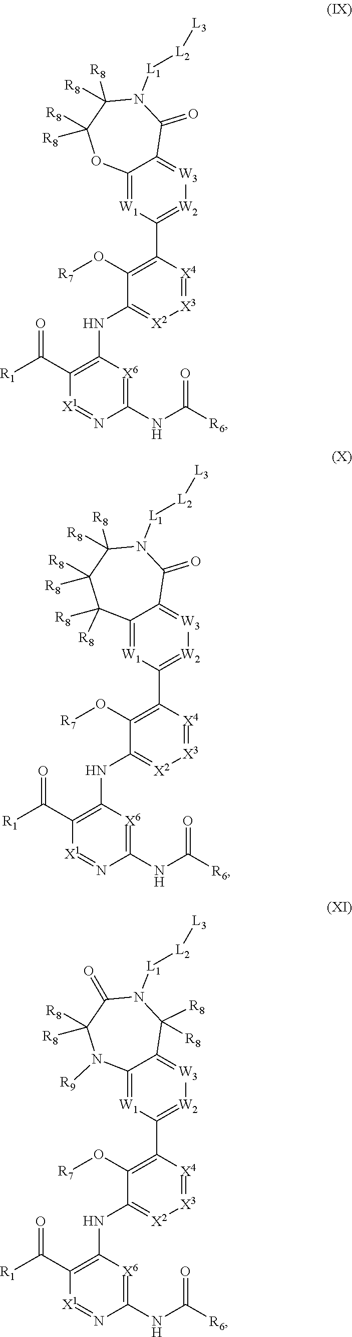

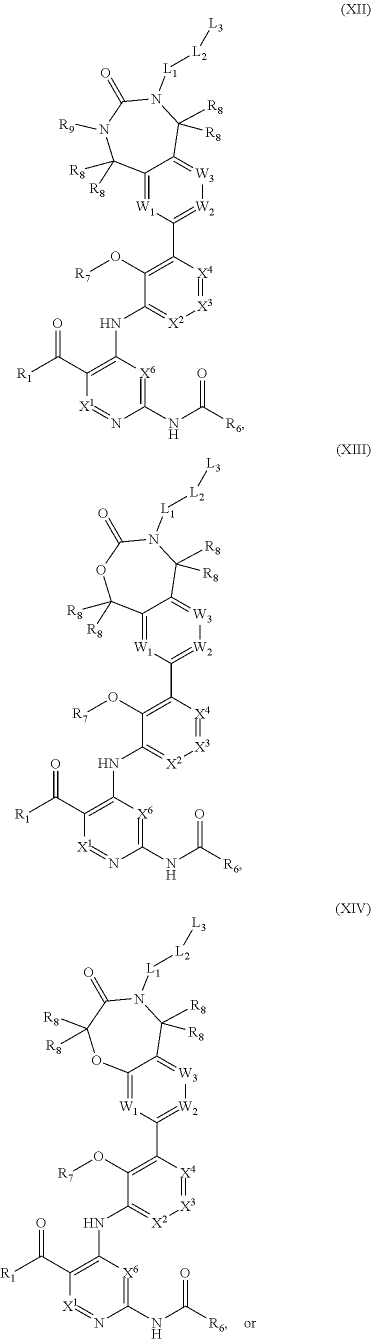

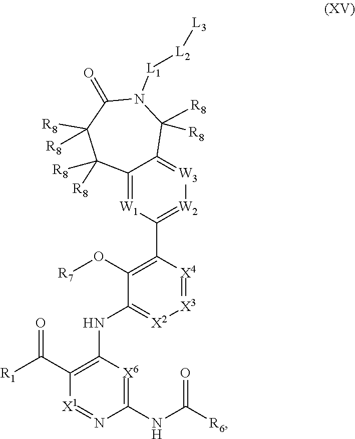

or a pharmaceutically acceptable salt or solvate or N-oxide thereof; wherein R₁, R₆, R₇, R₈, R₉, W₁, W₂, W₃, X₁, X₂, X₃, X₄, and X₆ are as defined herein; and wherein
L¹ is absent, —(CR^{c}R^{d})ₚ—, —S(O)—, —S(O)₂—, or —C(═O)—;
L² is absent, aryl or heteroaryl; and
L³ is H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, and heterocycloalkenyl groups of L² and L³ are optionally substituted by 1-6 R^{f} groups.

In some embodiments, the compound of Formula (I) is a compound of Formula (VI)

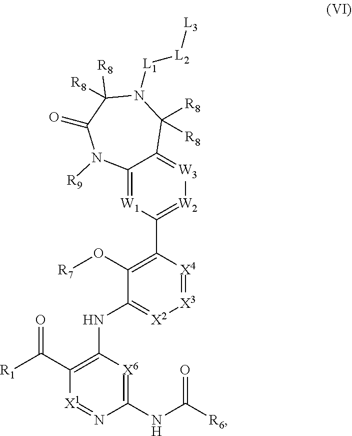

or a pharmaceutically acceptable salt or solvate or N-oxide thereof; wherein R₁, R₆, R₇, R₈, R₉, L₁, L₂, L₃, W₁, W₂, W₃, X₁, X₂, X₃, X₄, and X₆ are as defined herein.

In some embodiments, the compound of Formula (I) is a compound of Formula (VII)

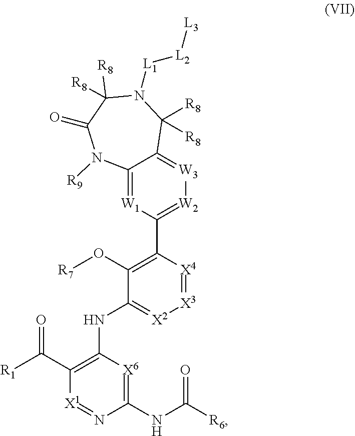

or a pharmaceutically acceptable salt or solvate or N-oxide thereof; wherein R₁, R₆, R₇, R₈, R₉, L₁, L₂, L₃, W₁, W₂, W₃, X₁, X₂, X₃, X₄, and X₆ are as defined herein.

In some embodiments, the compound of Formula (I) is a compound of Formula (VIII)

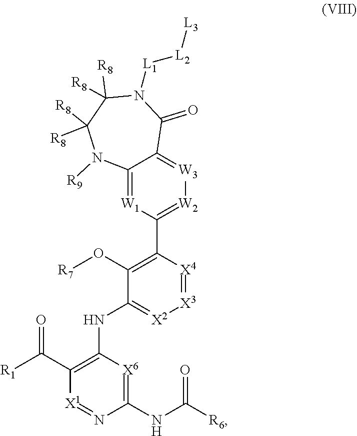

or a pharmaceutically acceptable salt or solvate or N-oxide thereof; wherein R₁, R₆, R₇, R₈, R₉, L₁, L₂, L₃, W₁, W₂, W₃, X₁, X₂, X₃, X₄, and X₆ are as defined herein.

In some embodiments, the compound of Formula (I) is a compound of Formula (XIX)

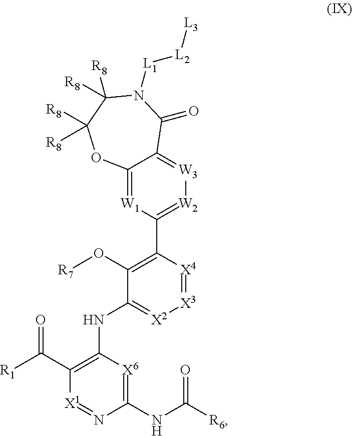

or a pharmaceutically acceptable salt or solvate or N-oxide thereof; wherein R₁, R₆, R₇, R₈, L₁, L₂, L₃, W₁, W₂, W₃, X₁, X₂, X₃, X₄, and X₆ are as defined herein.

In other embodiments, the compound of Formula (I) is a compound of Formula (X)

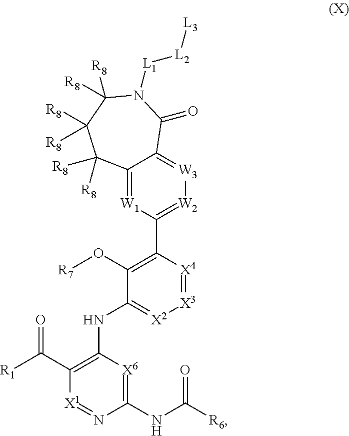

or a pharmaceutically acceptable salt or solvate or N-oxide thereof; wherein R₁, R₆, R₇, R₈, L₁, L₂, L₃, W₁, W₂, W₃, X₁, X₂, X₃, X₄, and X₆ are as defined herein.

In other embodiments, the compound of Formula (I) is a compound of Formula (XI)

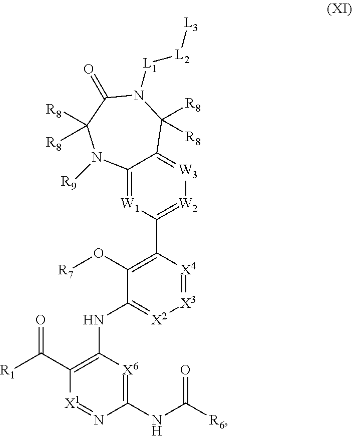

or a pharmaceutically acceptable salt or solvate or N-oxide thereof; wherein R₁, R₆, R₇, R₈, R₉, L₁, L₂, L₃, W₁, W₂, W₃, X₁, X₂, X₃, X₄, and X₆ are as defined herein.

In other embodiments, the compound of Formula (I) is a compound of Formula (XII)

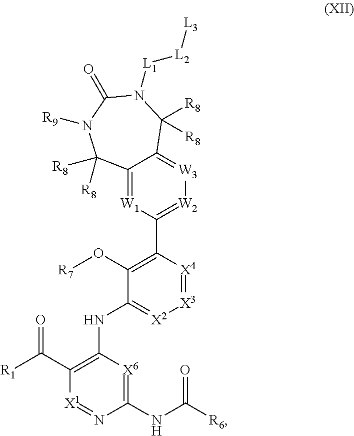

or a pharmaceutically acceptable salt or solvate or N-oxide thereof; wherein R₁, R₆, R₇, R₈, L₁, L₂, L₃, W₁, W₂, W₃, X₁, X₂, X₃, X₄, and X₆ are as defined herein.

In other embodiments, the compound of Formula (I) is a compound of Formula (XIII)

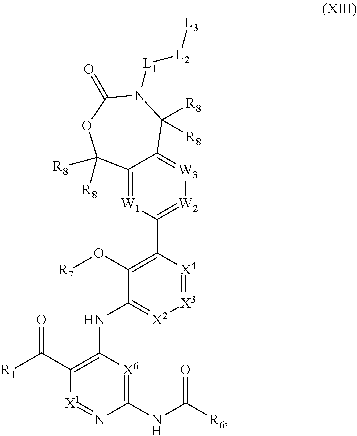

or a pharmaceutically acceptable salt or solvate or N-oxide thereof; wherein R₁, R₆, R₇, R₈, L₁, L₂, L₃, W₁, W₂, W₃, X₁, X₂, X₃, X₄, and X₆ are as defined herein.

In yet other embodiments, the compound of Formula (I) is a compound of Formula (XIV)

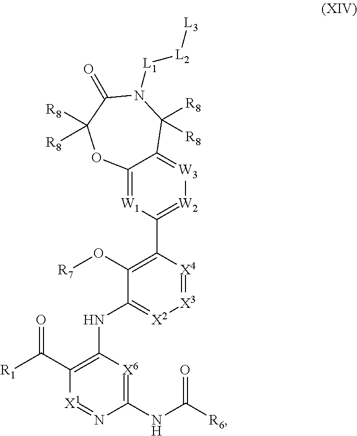

or a pharmaceutically acceptable salt or solvate or N-oxide thereof; wherein R₁, R₆, R₇, R₈, L₁, L₂, L₃, W₁, W₂, W₃, X₁, X₂, X₃, X₄, and X₆ are as defined herein.

In yet other embodiments, the compound of Formula (I) is a compound of Formula (XV)

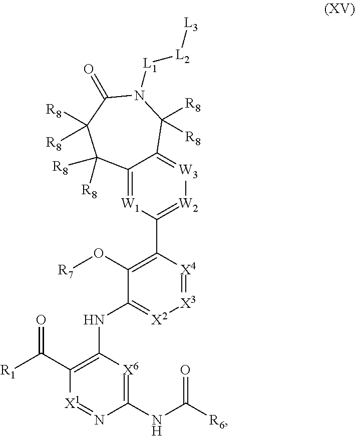

or a pharmaceutically acceptable salt or solvate or N-oxide thereof; wherein R₁, R₆, R₇, R₈, L₁, L₂, L₃, W₁, W₂, W₃, X₁, X₂, X₃, X₄, and X₆ are as defined herein.

In some embodiments, the compound of Formula (I) is a compound that is:
6-(Cyclopropanecarboxamido)-4-((3-(1-ethyl-4-((6-(dimethylcarbamoyl)pyridin-2-yl)methyl)-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-isopropyl-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-(3-(dimethylcarbamoyl)-2-fluorobenzyl)-1-ethyl-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(1-cyclopropyl-4-((6-(dimethylcarbamoyl)pyridin-2-yl)methyl)-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(2-((6-(dimethylcarbamoyl)pyridin-2-yl)methyl)-1-oxo-2,3,4,5-tetrahydro-1H-benzo[c]azepin-7-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-5-oxo-2,3,4,5-tetrahydrobenzo[f][1,4]oxazepin-8-yl)-2-methoxyphenyl)amino)-N-methylpyridazine-3-carboxamide; 6-(Cyclopropanecarboxamido)-4-((3-(4-((6-(dimethylcarbamoyl)pyridin-2-yl)methyl)-6-fluoro-5-oxo-2,3,4,5-tetrahydrobenzo[f][1,4]oxazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-methyl-5-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-ethyl-5-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-isopropyl-5-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-methyl-2,5-dioxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-ethyl-2,5-dioxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-isopropyl-2,5-dioxo-2,3,4,5-tetrahydro-1H-benzo[e][1,4]diazepin-8-yl)-2-methoxyphenyl)amino)-N-methylpyridazine-3-carboxamide;
6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
3-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-2-fluoro-N,N-dimethylbenzamide;
6-((8-(3-((5-Acetyl-2-(cyclopropanecarboxamido)pyridin-4-yl)amino)-2-methoxyphenyl)-1-(2-fluoroethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(2,2,2-trifluoroethyl)-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(cyanomethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(oxetan-3-yl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(3-((5-Acetyl-2-((1R,2S)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
(S)-6-((8-(3-((5-Acetyl-2-(spiro[2.2]pentane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
(R)-6-((8-(3-((5-Acetyl-2-(spiro[2.2]pentane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-((1S,2R)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-((1R,2R)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-((1S,2S)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-cyclopropyl-2-oxo-1,2,3,5-tetrahydro-4H-benzo[e][1,4]diazepin-4-yl)methyl)-N,N-dimethylpicolinamide;
   or a pharmaceutically acceptable salt thereof.

In some embodiments, the compound of Formula (I) is a compound that is:
6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(S)-6-((8-(3-((5-acetyl-2-(cyclopropanecarboxamido)pyridin-4-yl)amino)-5-fluoro-2-methoxyphenyl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-(2-fluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-isopropyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-isobutyl-2-oxo-1,2,3,5-tetrahydro-4H-pyrido[3,2-e][1,4]diazepin-4-yl)methyl)-N,N-dimethylpicolinamide;
   or a pharmaceutically acceptable salt thereof.

In some embodiments, the compound of Formula (I) is a compound that is:
6-((8-(3-((5-acetyl-2-(cyclopropanecarboxamido)pyridin-4-yl)amino)-2-methoxyphenyl)-1-cyclobutyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(1-fluoropropan-2-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(thiazol-5-ylmethyl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(cyclopropylmethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-cyclopentyl-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-2-oxo-1-((S)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-2-oxo-1-((S)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-(2-fluoroethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2R)-2-methylcyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2S)-2-methylcyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-(2-fluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]-diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-cyclobutyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-6-fluoro-2-oxo-1-((S)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-2-oxo-1-((R)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-(2,2-difluoroethyl)-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-(2-fluoroethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-6-fluoro-2-methoxy-phenyl)-1-ethyl-2-oxo-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxy-5-(trifluoromethyl)phenyl)-1-methyl-2-oxo-1,2,3,5-tetrahydro-4H-pyrido[3,2-e][1,4]-diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxy-5-(trifluoromethyl)phenyl)-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-2-oxo-1-((S)-tetrahydrofuran-3-yl)-1,2,3,5-tetrahydro-4H-pyrido[3,2-e][1,4]-diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-2-oxo-1-((S)-tetrahydrofuran-3-yl)-1,2,3,5-tetrahydro-4H-pyrido[3,2-e][1,4]-diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-1-(2-fluoro-2-methylpropyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-1-(2-methoxy-2-methylpropyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-1-isobutyl-2-oxo-1,2,3,5-tetrahydro-4H-pyrido[3,2-e][1,4]diazepin-4-yl)methyl)-N,N-dimethylpicolinamide; 3-((8-(3-((5-acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N,N-dimethylbenzamide;
6-((8-(3-((5-Acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N-methyl-N-(2,2,2-trifluoroethyl)picolinamide;
N-(5-acetyl-4-((3-(1-(2,2-difluoroethyl)-4-((6-(3-hydroxypyrrolidine-1-carbonyl)pyridin-2-yl)methyl)-2-oxo-2,3,4,5-tetrahydro-1H-pyrido[3,2-e][1,4]diazepin-8-yl)-5-fluoro-2-methoxy-phenyl)amino)pyridin-2-yl)cyclopropanecarboxamide;
N-(5-acetyl-4-((3-(1-(2,2-difluoroethyl)-2-oxo-4-((6-(pyrrolidine-1-carbonyl)pyridin-2-yl)methyl)-2,3,4,5-tetrahydro-1H-pyrido[3,2-e][1,4]diazepin-8-yl)-5-fluoro-2-methoxyphenyl)-amino)pyridin-2-yl)cyclopropanecarboxamide;
6-((8-(3-((5-acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N-(2,2-difluoroethyl)-N-methylpicolinamide;
6-((8-(3-((5-acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N-(2-hydroxyethyl)-N-methylpicolinamide;
6-((8-(3-((5-acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N-(tert-butyl)-N-methylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-6-fluoro-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-6-fluoro-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-6-fluoro-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxy-5-methyl-phenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxy-5-(trifluoromethyl)phenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-4-fluoro-2-methoxy-phenyl)-1-methyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(R)-6-((8-(3-((5-acetyl-2-(cyclopropanecarboxamido)pyridin-4-yl)amino)-2-methoxy-phenyl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
(S)-6-((8-(3-((5-acetyl-2-(cyclopropanecarboxamido)pyridin-4-yl)amino)-2-methoxy-phenyl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-isopropyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-ethyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-1-isopropyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-((1s,3s)-3-cyanocyclobutyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(1-cyanoethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxy-5-(trifluoromethyl)phenyl)-1-(2,2-difluoroethyl)-2-oxo-1,2,3,5-tetrahydro-4H-pyrido[3,2-e][1,4]-diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-ethyl-7-fluoro-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2-cyanoethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-methyl-2-oxo-1,2,3,5-tetrahydro-4H-pyrido[4,3-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-1-(2-methoxyethyl)-2-oxo-1,2,3,5-tetrahydro-4H-pyrido[4,3-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-pyrido[4,3-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-isopropyl-2-oxo-1,2,3,5-tetrahydro-4H-pyrido[4,3-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[4,3-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2,2-difluoropropyl)-2-oxo-1,2,3,5-tetrahydro-4H-pyrido[3,2-e][1,4]diazepin-4-yl)methyl)-N,N-dimethylpicolinamide;
(R)-6-((8-(3-((5-acetyl-2-(cyclopropanecarboxamido)pyridin-4-yl)amino)-2-methoxy-phenyl)-1-(1-methoxypropan-2-yl)-2-oxo-1,2,3,5-tetrahydro-4H-pyrido[3,2-e][1,4]diazepin-4-yl)methyl)-N,N-dimethylpicolinamide;
(S)-6-((8-(3-((5-acetyl-2-(cyclopropanecarboxamido)pyridin-4-yl)amino)-2-methoxy-phenyl)-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(2,2-difluoroethyl)-6-fluoro-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-ethyl-6-fluoro-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-methyl-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-isopropyl-2-oxo-1,2,3,5-tetrahydro-4H-benzo[e][1,4]diazepin-4-yl)methyl)-N,N-dimethylpicolinamide;
6-((8-(2-((5-acetyl-2-(cyclopropanecarboxamido)pyridin-4-yl)amino)-3-methoxypyridin-4-yl)-1-isopropyl-2-oxo-1,2,3,5-tetrahydro-4H-benzo[e][1,4]diazepin-4-yl)methyl)-N,N-dimethylpicolinamide;
6-((8-(4-((5-acetyl-2-((1R,2S)-2-fluorocyclopropane-1-carboxamido)pyridin-4-yl)amino)-3-methoxypyridin-2-yl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-((1S,2S)-2-methylcyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]-diazepin-4-yl)methyl)-N,N-dimethylpicolinamide;
6-((8-(3-((5-acetyl-2-((1R,2R)-2-methylcyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]-diazepin-4-yl)methyl)-N,N-dimethylpicolinamide;
6-(8-(3-((5-acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-2-oxo-1-((S)-tetrahydrofuran-3-yl)-1,2,3,5-tetrahydro-4H-benzo[e][1,4]-diazepin-4-yl)methyl)-N,N-dimethylpicolinamide;
6-((8-(2-((5-acetyl-2-(cyclopropanecarboxamido)pyridin-4-yl)amino)-3-methoxypyridin-4-yl)-1-isopropyl-2-oxo-1,2,3,5-tetrahydro-4H-pyrido[3,2-e][1,4]diazepin-4-yl)methyl)-N,N-dimethylpicolinamide;
6-((8-(2-((5-acetyl-2-(cyclopropanecarboxamido)pyridin-4-yl)amino)-3-methoxypyridin-4-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-neopentyl-2-oxo-1,2,3,5-tetrahydro-4H-pyrido[3,2-e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(2,2-difluoroethyl)-2-oxo-1,2,3,5-tetrahydro-4H-benzo[e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2S)-2-methylcyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-methylcyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1R,2R)-2-methylcyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1R,2S)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2S)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1R,2R)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((2-(cyclopropanecarboxamido)-5-propionylpyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-chloro-2-methoxy-phenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(R)-6-((8-(4-((5-acetyl-2-(cyclopropanecarboxamido)pyridin-4-yl)amino)-3-methoxypyridin-2-yl)-1-(1-methoxypropan-2-yl)-2-oxo-1,2,3,5-tetrahydro-4H-pyrido[3,2-e][1,4]diazepin-4-yl)methyl)-N,N-dimethylpicolinamide;
(1R,2S)—N-(5-acetyl-4-((5-fluoro-2-methoxy-3-(1-methyl-4-((6-(morpholine-4-carbonyl)-pyridin-2-yl)methyl)-2-oxo-2,3,4,5-tetrahydro-1H-benzo[e][1,4]diazepin-8-yl)phenyl)amino)-pyridin-2-yl)-2-fluorocyclopropane-1-carboxamide;
N-(5-acetyl-4-((3-(4-((6-(3,3-difluoropyrrolidine-1-carbonyl)pyridin-2-yl)methyl)-1-ethyl-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-((1-methyl-1H-pyrazol-4-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-((6-((3aR,6aR)-hexahydro-1H-furo[3,4-c]pyrrole-5-carbonyl)-pyridin-2-yl)methyl)-2-oxo-2,3,4,5-tetrahydro-1H-benzo[e][1,4]diazepin-8-yl)-2-methoxyphenyl)-amino)pyridin-2-yl)cyclopropanecarboxamide;
N-(5-acetyl-4-((3-(1-ethyl-4-((6-(morpholine-4-carbonyl)pyridin-2-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(4-((3-(4-([1,2,4]triazolo[1,5-a]pyridin-5-ylmethyl)-1-ethyl-2-oxo-2,3,4,5-tetrahydro-1H-benzo[e][1,4]diazepin-8-yl)-2-methoxyphenyl)amino)-5-acetylpyridin-2-yl)cyclopropanecarboxamide;
N-(4-((3-(4-([1,2,4]triazolo[1,5-a]pyridin-8-ylmethyl)-1-ethyl-2-oxo-2,3,4,5-tetrahydro-1H-benzo[e][1,4]diazepin-8-yl)-2-methoxyphenyl)amino)-5-acetylpyridin-2-yl)cyclopropanecarboxamide;
N-(5-acetyl-4-((3-(1-ethyl-4-(imidazo[1,5-a]pyridin-8-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-(imidazo[1,2-a]pyridin-8-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-2-oxo-4-(pyrazolo[1,5-a]pyridin-7-ylmethyl)-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-(imidazo[1,2-a]pyridin-5-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-((6-(methylsulfonyl)pyridin-2-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-(isoquinolin-1-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(4-((4-cyanopyridin-2-yl)methyl)-1-ethyl-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-(2,2-difluoroethyl)-4-(imidazo[1,5-a]pyridin-8-ylmethyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-(imidazo[1,2-a]pyridin-8-ylmethyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-(3-(difluoromethyl)-2-fluorobenzyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(4-(3-chlorobenzyl)-1-(2,2-difluoroethyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-((1-methyl-2-oxo-1,2-dihydropyridin-3-yl)methyl)-2-oxo-2,3,4,5-tetrahydro-1H-pyrido[3,2-e][1,4]diazepin-8-yl)-2-methoxyphenyl)amino)-pyridin-2-yl)cyclopropanecarboxamide;
N-(5-acetyl-4-((3-(1-(2,2-difluoroethyl)-2-oxo-4-((6-oxo-1,6-dihydropyridin-2-yl)methyl)-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-((6-(difluoromethyl)pyridin-2-yl)methyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-2-oxo-4-(pyridin-2-ylmethyl)-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-2-oxo-4-(pyridin-3-ylmethyl)-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-4-((6-(hydroxymethyl)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-2-oxo-4-(thiazol-4-ylmethyl)-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
6-((8-(3-((5-acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N-methoxy-N-methylpicolinamide;
3-((8-(3-((5-acetyl-2-(cyclopropanecarboxamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N-methoxy-N-methylbenzamide;
N-(5-acetyl-4-((3-(4-((6-aminopyridin-2-yl)methyl)-1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-4-((6-(3-methylureido)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-4-((6-((N-methylsulfamoyl)amino)pyridin-2-yl)methyl)-2-oxo-2,3,4,5-tetrahydro-1H-pyrido[3,2-e][1,4]diazepin-8-yl)-5-fluoro-2-methoxy-phenyl)amino)pyridin-2-yl)cyclopropanecarboxamide;
N-(5-Acetyl-4-((2-methoxy-3-(1-methyl-2-oxo-4-((6-(tetrahydrofuran-2-yl)pyridin-2-yl)methyl)-2,3,4,5-tetrahydro-1H-benzo[e][1,4]diazepin-8-yl)phenyl)amino)pyridin-2-yl)cyclopropanecarboxamide;
N-(5-Acetyl-4-((2-methoxy-3-(4-((6-(3-methoxyoxetan-3-yl)pyridin-2-yl)methyl)-1-methyl-2-oxo-2,3,4,5-tetrahydro-1H-benzo[e][1,4]diazepin-8-yl)phenyl)amino)pyridin-2-yl)cyclopropanecarboxamide;
N-(5-Acetyl-4-((3-(4-((6-acetylpyridin-2-yl)methyl)-1-methyl-2-oxo-2,3,4,5-tetrahydro-1H-benzo[e][1,4]diazepin-8-yl)-2-methoxyphenyl)amino)pyridin-2-yl)cyclopropanecarboxamide;
6-(2-(8-(3-((5-Acetyl-2-(cyclopropanecarboxamido)pyridin-4-yl)amino)-2-methoxyphenyl)-1-methyl-2-oxo-1,2,3,5-tetrahydro-4H-benzo[e][1,4]diazepin-4-yl)propan-2-yl)-N,N-dimethylpicolinamide;
6-((8-(3-((5-Acetyl-2-(cyclopropanecarboxamido)pyridin-4-yl)amino)-2-methoxyphenyl)-1-(tert-butyl)-2-oxo-1,2,3,5-tetrahydro-4H-pyrido[3,2-e][1,4]diazepin-4-yl)methyl)-N,N-dimethylpicolinamide;
(S)-6-((8-(3-((5-Acetyl-2-(cyclopropanecarboxamido)pyridin-4-yl)amino)-2-methoxy-phenyl)-1-(1-acetylpyrrolidin-3-yl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(azetidin-3-yl)-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxy-6-(trifluoromethyl)pyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxy-6-(trifluoromethyl)pyridin-2-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(4-((5-acetyl-2-((1S,2S)-2-methylcyclopropane-1-carboxamido)pyridin-4-yl)amino)-3-methoxy-6-(trifluoromethyl)pyridin-2-yl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
(S)-6-((8-(2-((5-Acetyl-2-(cyclopropanecarboxamido)pyridin-4-yl)amino)-3-methoxy-6-(trifluoromethyl)pyridin-4-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-(bicyclo[1.1.1]pentan-1-yl)-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(3-fluorobicyclo[1.1.1]pentan-1-yl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-(bicyclo[1.1.1]pentan-1-yl)-2-oxo-1,2,3,5-tetrahydro-4H-pyrido[3,2-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(2-oxabicyclo[2.1.1]hexan-4-yl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-isobutyl-2-oxo-1,2,3,5-tetrahydro-4H-pyrido[3,2-e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-cyclopentyl-2-oxo-1,2,3,5-tetrahydro-4H-pyrido[3,2-e][1,4]diazepin-4-yl)methyl)-N,N-dimethylpicolinamide;
6-((8-(3-((5-Acetyl-2-((1R,2S)-2-fluorocyclopropane-1-carboxamido)pyridin-4-yl)amino)-2-methoxyphenyl)-2-oxo-1-((S)-tetrahydro-2H-pyran-3-yl)-1,2,3,5-tetrahydro-4H-pyrido[3,2-e][1,4]-diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-1-(sec-butyl)-2-oxo-1,2,3,5-tetrahydro-4H-pyrido[3,2-e][1,4]diazepin-4-yl)methyl)-N,N-dimethylpicolinamide;
(R)-6-((8-(4-((5-acetyl-2-(cyclopropanecarboxamido)pyridin-4-yl)amino)-3-methoxypyridin-2-yl)-1-(sec-butyl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-((R)-sec-butyl)-2-oxo-1,2,3,5-tetrahydro-4H-pyrido[3,2-e][1,4]diazepin-4-yl)methyl)-N,N-dimethylpicolinamide;
5-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylbicyclo[3.1.1]heptane-1-carboxamide;
6-((8-(4-((5-Acetyl-2-(cyclopropanecarboxamido)pyridin-4-yl)amino)-3-methoxypyridin-2-yl)-1-cyclopentyl-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-2-oxo-1-propyl-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-2-oxo-1-(pentan-3-yl)-1,2,3,5-tetrahydro-4H-pyrido[3,2-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-cyclohexyl-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-cyclohexyl-2-oxo-1,2,3,5-tetrahydro-4H-pyrido[3,2-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(tetrahydro-2H-pyran-4-yl)-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-5-fluoro-2-methoxy-phenyl)-1-(2-hydroxy-2-methylpropyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-cyclopropyl-2-oxo-1,2,3,5-tetrahydro-4H-benzo[e][1,4]diazepin-4-yl)methyl)-N,N-dimethylpicolinamide;
3-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-2-methoxy-N,N-dimethylbenzamide;
3-((8-(3-((5-acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N,2-trimethylbenzamide;
6-(8-(3-((5-Acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylpicolinamide;
5-(8-(3-((5-Acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylbicyclo[3.1.1]heptane-1-carboxamide;
6-((1-(4-(1H-Pyrazol-1-yl)phenyl)-8-(3-((5-acetyl-2-(cyclopropanecarboxamido)pyridin-4-yl)amino)-2-methoxyphenyl)-2-oxo-1,2,3,5-tetrahydro-4H-benzo[e][1,4]diazepin-4-yl)methyl)-N,N-dimethylpicolinamide;
6-((1-(6-(1H-Pyrazol-1-yl)pyridin-3-yl)-8-(3-((5-acetyl-2-(cyclopropanecarboxamido)-pyridin-4-yl)amino)-2-methoxyphenyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(6-methoxypyridin-3-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2-fluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3,4-difluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(quinolin-7-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(quinolin-6-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(5-fluoropyridin-2-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3-fluoropyridin-4-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2,4-difluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(4-cyanophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-phenyl-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3-chlorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3-fluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(6-methylpyridin-3-yl)-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-2-oxo-1-(quinolin-3-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(4-fluorophenyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(thiazol-5-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(pyridin-2-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(pyridin-4-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(5-fluoropyridin-3-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2-methoxypyridin-4-yl)-2-oxo-1,2,3,5-tetrahydro-4H-benzo[e][1,4]diazepin-4-yl)methyl)-N,N-dimethylpicolinamide;
N-(5-acetyl-4-((2-methoxy-3-(4-((6-(morpholine-4-carbonyl)pyridin-2-yl)methyl)-2-oxo-1-(pyridin-3-yl)-2,3,4,5-tetrahydro-1H-benzo[e][1,4]diazepin-8-yl)phenyl)amino)pyridin-2-yl)cyclopropanecarboxamide;
6-((8-(3-((5-acetyl-2-(cyclopropanecarboxamido)pyridin-4-yl)amino)-2-methoxyphenyl)-1-(methylsulfonyl)-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-6-cyano-1-isopropyl-2-oxo-1,2,3,5-tetrahydro-4H-benzo[e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-1-(1,1-difluoropropan-2-yl)-2-oxo-1,2,3,5-tetrahydro-4H-pyrido[3,2-e][1,4]diazepin-4-yl)methyl)-N,N-dimethylpicolinamide;
(R)-6-((8-(4-((5-Acetyl-2-(cyclopropanecarboxamido)pyridin-4-yl)amino)-3-methoxy-pyridin-2-yl)-1-(1,1-difluoropropan-2-yl)-2-oxo-1,2,3,5-tetrahydro-4H-pyrido[3,2-e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-6-(ethylamino)-1-isopropyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-pyrido[2,3-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-6-ethoxy-1-ethyl-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-6-ethoxy-1-ethyl-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-isopropyl-6-methoxy-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-6-chloro-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)sulfonyl)-N,N-dimethylpicolinamide;
N-(5-Acetyl-4-((2-methoxy-3-(1-methyl-4-((6-(morpholine-4-carbonyl)pyridin-2-yl)sulfonyl)-2-oxo-2,3,4,5-tetrahydro-1H-benzo[e][1,4]diazepin-8-yl)phenyl)amino)pyridin-2-yl)cyclopropanecarboxamide;
6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)sulfonyl)-N,N-dimethylpicolinamide;
(R)-6-((8-(3-((5-Acetyl-2-(cyclopropanecarboxamido)pyridin-4-yl)amino)-2-methoxy-phenyl)-1-ethyl-3-methyl-2-oxo-1,2,3,5-tetrahydro-4H-benzo[e][1,4]diazepin-4-yl)methyl)-N,N-dimethylpicolinamide;
(S)-6-((8-(3-((5-Acetyl-2-(cyclopropanecarboxamido)pyridin-4-yl)amino)-2-methoxy-phenyl)-1-ethyl-3-methyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-ethyl-5-methyl-2-oxo-1,2,3,5-tetrahydro-4H-benzo[e][1,4]diazepin-4-yl)methyl)-N,N-dimethylpicolinamide;
6-((8-(3-((2-(Cyclopropanecarboxamido)-5-(2-fluoroacetyl)pyridin-4-yl)amino)-2-methoxy-phenyl)-1-ethyl-2-oxo-1,2,3,5-tetrahydro-4H-benzo[e][1,4]diazepin-4-yl)methyl)-N,N-dimethylpicolinamide;
6-((8-(3-((2-(Cyclopropanecarboxamido)-5-(2,2-difluoroacetyl)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyrimidin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((3-Acetyl-6-(cyclopropanecarboxamido)pyridazin-4-yl)amino)-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((2-(Cyclopropanecarboxamido)-5-oxo-5,6-dihydro-1,6-naphthyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-acetyl-2-(2-cyclopropylacetamido)pyridin-4-yl)amino)-5-fluoro-2-methoxy-phenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2R)-2-(trifluoromethyl)cyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido-[3,2-e][1,4]diazepin-4-yl)methyl)-N,N-dimethylpicolinamide;
(S)-6-((8-(3-((5-acetyl-2-(2,2-difluorocyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]-diazepin-4-yl)methyl)-N,N-dimethylpicolinamide;
(R)-6-((8-(3-((5-acetyl-2-(2,2-difluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-acetyl-2-(2,2-dimethylcyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]-diazepin-4-yl)methyl)-N,N-dimethylpicolinamide;
6-((5-((1-((3′,6′-dihydroxy-3-oxo-3H-spiro[isobenzofuran-1,9′-xanthen]-5-yl)amino)-1-thioxo-5,8,11-trioxa-2-azatridecan-13-yl)carbamoyl)pyridin-2-yl)amino)-4-((3-(1-(1-((6-(dimethylcarbamoyl)pyridin-2-yl)methyl)azetidin-3-yl)-1H-pyrazol-4-yl)-2-methoxyphenyl)amino)-N-methylpyridazine-3-carboxamide;
   or a pharmaceutically acceptable salt thereof.

In yet further embodiments, the compounds of Formula (I) are in the form of a pharmaceutically acceptable salt.

In yet further embodiments, disclosed is a pharmaceutical composition comprising a compound according to Formula (I), or a pharmaceutically acceptable salt thereof, and a pharmaceutically acceptable excipient.

It will be apparent that the compounds of Formula I, including all subgenera described herein, may have multiple stereogenic centers. As a result, there exist multiple stereoisomers (enantiomers and diastereomers) of the compounds of Formula I (and subgenera described herein).

The present disclosure contemplates and encompasses each stereoisomer of any compound of Formula I (and subgenera described herein), as well as mixtures of said stereoisomers.

Pharmaceutically acceptable salts and solvates of the compounds of Formula I (including all subgenera described herein) are also within the scope of the disclosure.

Isotopic variants of the compounds of Formula I (including all subgenera described herein) are also contemplated by the present disclosure.

### Pharmaceutical Compositions and Methods of Administration

In some embodiments, the disclosure is directed to pharmaceutical compositions comprising compounds of Formula I, or a pharmaceutically acceptable salt or solvate thereof.

The subject pharmaceutical compositions are typically formulated to provide a therapeutically effective amount of a compound of the present disclosure as the active ingredient, or a pharmaceutically acceptable salt, ester, prodrug, solvate, hydrate or derivative thereof. Where desired, the pharmaceutical compositions contain pharmaceutically acceptable salt and/or coordination complex thereof, and one or more pharmaceutically acceptable excipients, carriers, including inert solid diluents and fillers, diluents, including sterile aqueous solution and various organic solvents, permeation enhancers, solubilizers and adjuvants.

The subject pharmaceutical compositions can be administered alone or in combination with one or more other agents, which are also typically administered in the form of pharmaceutical compositions. Where desired, the one or more compounds of the invention and other agent(s) may be mixed into a preparation or both components may be formulated into separate preparations to use them in combination separately or at the same time.

In some embodiments, the concentration of one or more compounds provided in the pharmaceutical compositions of the present invention is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v or v/v.

In some embodiments, the concentration of one or more compounds of the invention is greater than 90%, 80%, 70%, 60%, 50%, 40%, 30%, 20%, 19.75%, 19.50%, 19.25%, 19%, 18.75%, 18.50%, 18.25% 18%, 17.75%, 17.50%, 17.25% 17%, 16.75%, 16.50%, 16.25%, 16%, 15.75%, 15.50%, 15.25% 15%, 14.75%, 14.50%, 14.25% 14%, 13.75%, 13.50%, 13.25%, 13%, 12.75%, 12.50%, 12.25%, 12%, 11.75%, 11.50%, 11.25% 11%, 10.75%, 10.50%, 10.25% 10%, 9.75%, 9.50%, 9.25%, 9%, 8.75%, 8.50%, 8.25% 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, or 0.0001% (or a number in the range defined by and including any two numbers above) w/w, w/v, or v/v.

In some embodiments, the concentration of one or more compounds of the invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v or v/v.

In some embodiments, the concentration of one or more compounds of the invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w, w/v or v/v.

In some embodiments, the amount of one or more compounds of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g (or a number in the range defined by and including any two numbers above).

In some embodiments, the amount of one or more compounds of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g (or a number in the range defined by and including any two numbers above).

In some embodiments, the amount of one or more compounds of the invention is in the range of 0.0001-10 g, 0.0005-9 g, 0.001-8 g, 0.005-7 g, 0.01-6 g, 0.05-5 g, 0.1-4 g, 0.5-4 g, or 1-3 g.

The compounds according to the invention are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. An exemplary dosage is 10 to 30 mg per day. The exact dosage will depend upon the route of administration, the form in which the compound is administered, the subject to be treated, the body weight of the subject to be treated, and the preference and experience of the attending physician.

A pharmaceutical composition of the invention typically contains an active ingredient (i.e., a compound of the disclosure) of the present invention or a pharmaceutically acceptable salt and/or coordination complex thereof, and one or more pharmaceutically acceptable excipients, carriers, including but not limited to inert solid diluents and fillers, diluents, sterile aqueous solution and various organic solvents, permeation enhancers, solubilizers and adjuvants.

Described below are non-limiting exemplary pharmaceutical compositions and methods for preparing the same.

### Pharmaceutical Compositions for Oral Administration.

In some embodiments, the invention provides a pharmaceutical composition for oral administration containing a compound of the invention, and a pharmaceutical excipient suitable for oral administration.

In some embodiments, the invention provides a solid pharmaceutical composition for oral administration containing: (i) an effective amount of a compound of the invention; optionally (ii) an effective amount of a second agent; and (iii) a pharmaceutical excipient suitable for oral administration. In some embodiments, the composition further contains: (iv) an effective amount of a third agent.

In some embodiments, the pharmaceutical composition may be a liquid pharmaceutical composition suitable for oral consumption. Pharmaceutical compositions of the invention suitable for oral administration can be presented as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

This invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

An active ingredient can be combined in an intimate admixture with a pharmaceutical carrier according to conventional pharmaceutical compounding techniques. The carrier can take a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrants may be used in the compositions of the invention to provide tablets that disintegrate when exposed to an aqueous environment. Too much of a disintegrant may produce tablets which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant, or about 1 to about 5 weight percent of disintegrant, may be used in the pharmaceutical composition. Disintegrants that can be used to form pharmaceutical compositions and dosage forms of the invention include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

Lubricants which can be used to form pharmaceutical compositions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof, lysophospholipids and derivatives thereof, carnitine fatty acid ester salts; salts of alkylsulfates; fatty acid salts; sodium docusate; acyl lactylates; mono- and diacetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof, carnitine fatty acid ester salts; salts of alkylsulfates; fatty acid salts; sodium docusate; acylactylates; mono- and diacetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof, polyoxyethylated vitamins and derivatives thereof, polyoxyethylene-polyoxypropylene block copolymers; and mixtures thereof, polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-1Ooleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof, and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a subject using conventional techniques, such as distillation or evaporation. Thus, if present, the solubilizer can be in a weight ratio of 10%, 25%o, 50%), 100%o, or up to about 200%> by weight, based on the combined weight of the drug, and other excipients. If desired, very small amounts of solubilizer may also be used, such as 5%>, 2%>, 1%) or even less. Typically, the solubilizer may be present in an amount of about 1%> to about 100%, more typically about 5%> to about 25%> by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)-aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Pharmaceutical Compositions for Topical (e.g., Transdermal) Delivery.

In some embodiments, the invention provides a pharmaceutical composition for transdermal delivery containing a compound of the present invention and a pharmaceutical excipient suitable for transdermal delivery.

Compositions of the present invention can be formulated into preparations in solid, semisolid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation.

Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another exemplary formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 20037ybg; Goodman and Gilman, eds., The Pharmacological Basis of Therapeutics, Tenth Edition, McGraw Hill, 2001; Remingtons Pharmaceutical Sciences, 20th Ed., Lippincott Williams & Wilkins., 2000; Martindale, The Extra Pharmacopoeia, Thirty-Second Edition (The Pharmaceutical Press, London, 1999); all of which are incorporated by reference herein in their entirety.

Administration of the compounds or pharmaceutical composition of the present invention can be affected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to 7 g/day, preferably about 0.05 to about 2.5 g/day. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the invention is administered in a single dose.

Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the compounds of the invention may continue as long as necessary. In some embodiments, a compound of the invention is administered for more than 1, 2, 3, 4, 5, 6, 7, 14, or 28 days. In some embodiments, a compound of the invention is administered for less than 28, 14, 7, 6, 5, 4, 3, 2, or 1 day. In some embodiments, a compound of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of the compound of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of the compound of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis.

A variety of stent devices which may be used as described are disclosed, for example, in the following references, all of which are hereby incorporated by reference: U.S. Pat. Nos. 5,451,233; 5,040,548; 5,061,273; 5,496,346; 5,292,331; 5,674,278; 3,657,744; 4,739,762; 5,195,984; 5,292,331; 5,674,278; 5,879,382; 6,344,053.

The compounds of the invention may be administered in dosages. It is known in the art that due to intersubject variability in compound pharmacokinetics, individualization of dosing regimen is necessary for optimal therapy. Dosing for a compound of the invention may be found by routine experimentation in light of the instant disclosure.

When a compound of the invention is administered in a composition that comprises one or more agents, and the agent has a shorter half-life than the compound of the invention unit dose forms of the agent and the compound of the invention may be adjusted accordingly.

The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compound in sterile aqueous solutions, for example, aqueous propylene glycol or dextrose solutions. Such dosage forms can be suitably buffered, if desired.

### Methods of Use

The method typically comprises administering to a subject a therapeutically effective amount of a compound of the invention. The therapeutically effective amount of the subject combination of compounds may vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of proliferation or downregulation of activity of a target protein. The specific dose will vary depending on the particular compounds chosen, the dosing regimen to be followed, whether it is administered in combination with other compounds, timing of administration, the tissue to which it is administered, and the physical delivery system in which it is carried.

As used herein, the term “IC₅₀” refers to the half maximal inhibitory concentration of an inhibitor in inhibiting biological or biochemical function. This quantitative measure indicates how much of a particular inhibitor is needed to inhibit a given biological process (or component of a process, i.e., an enzyme, cell, cell receptor or microorganism) by half. In other words, it is the half maximal (50%) inhibitory concentration (IC) of a substance (50% IC, or IC₅₀). EC₅₀ refers to the plasma concentration required for obtaining 50% of a maximum effect in vivo.

In some aspects, the present disclosure provides a method of modulating JAK2 activity (e.g., in vitro or in vivo), comprising contacting a cell with a therapeutically effective amount of a compound as described herein or a pharmaceutically acceptable salt thereof.

In some aspects, the present disclosure provides a method of treating or preventing a disease or disorder disclosed herein in a subject in need thereof, comprising administering to the subject a therapeutically effective amount of a compound as described herein or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound as described herein or a pharmaceutically acceptable salt thereof, or a pharmaceutical composition of the present disclosure.

### Treatment of Disorders

Provided compounds are inhibitors of JAK2 and are therefore useful for treating one or more disorders associated with activity of JAK2 or mutants thereof. Thus, in certain embodiments, the present disclosure provides a method of treating a JAK2-mediated disorder in a subject, comprising administering a therapeutically effective amount of a compound of the present disclosure, or a pharmaceutically acceptable salt thereof, or a pharmaceutically acceptable composition of either of the foregoing, to a subject in need thereof. In certain embodiments, the present disclosure provides a method of treating a JAK2-mediated disorder in a subject comprising administering a therapeutically effective amount of a compound of the present disclosure, or a pharmaceutically acceptable composition thereof, to a subject in need thereof. In some embodiments, the subject has a mutant JAK2. In some embodiments, the subject has JAK2 containing a V617F mutations.

As used herein, the term “JAK2-mediated” disorders, diseases, and/or conditions means any disease or other deleterious condition in which JAK2 or a mutant thereof is known to play a role. Accordingly, another embodiment of the present disclosure relates to treating or lessening the severity of one or more diseases in which JAK2, or a mutant thereof, is known to play a role. Such JAK2-mediated disorders include, but are not limited to, cellular proliferative disorders (e.g. cancer). In some embodiments, the JAK2-mediated disorder is a disorder mediated by a mutant JAK2. In some embodiments, the JAK2-mediated disorder is a disorder mediated by a JAK2 containing a V617F mutations.

In some embodiments, the present disclosure provides a method for treating a cellular proliferative disease, said method comprising administering to a patient in need thereof a therapeutically effective amount of a compound of the present disclosure, or a pharmaceutically acceptable salt thereof, or a pharmaceutically acceptable composition of either of the foregoing. In some embodiments, the present disclosure provides a method for treating a cellular proliferative disease, said method comprising administering to a patient in need thereof a therapeutically effective amount of a compound of the present disclosure, or a pharmaceutically acceptable composition thereof.

In some embodiments, the method of treatment comprises the steps of: i) identifying a subject in need of such treatment; (ii) providing a disclosed compound, or a pharmaceutically acceptable salt thereof; and (iii) administering said provided compound in a therapeutically effective amount to treat, suppress and/or prevent the disease state or condition in a subject in need of such treatment. In some embodiments, the subject has a mutant JAK2. In some embodiments, the subject has JAK2 containing a V617F mutation.

In some embodiments, the method of treatment comprises the steps of: i) identifying a subject in need of such treatment; (ii) providing a composition comprising a disclosed compound, or a pharmaceutically acceptable salt thereof; and (iii) administering said composition in a therapeutically effective amount to treat, suppress and/or prevent the disease state or condition in a subject in need of such treatment. In some embodiments, the subject has a mutant JAK2. In some embodiments, the subject has JAK2 containing a V617F mutation.

Another aspect of the disclosure provides a compound according to the definitions herein, or a pharmaceutically acceptable salt thereof, or a pharmaceutical composition of either of the foregoing, for use in the treatment of a disorder described herein. Another aspect of the disclosure provides the use of a compound according to the definitions herein, or a pharmaceutically acceptable salt thereof, or a pharmaceutical composition of either of the foregoing, for the treatment of a disorder described herein. Similarly, the disclosure provides the use of a compound according to the definitions herein, or a pharmaceutically acceptable salt thereof, for the preparation of a medicament for the treatment of a disorder described herein.

### Cellular Proliferative Diseases

In some embodiments, the disorder is a cellular proliferative disease. In some embodiments, the cellular proliferative disease is cancer. In some embodiments, the cancer is a tumor. In some embodiments, the cancer is a hematopoietic cancer. In some embodiments, the cancer is a solid tumor. In some embodiments, the cellular proliferative disease is a tumor and/or cancerous cell growth. In some embodiments, the cellular proliferative disease is a tumor. In some embodiments, the cellular proliferative disease is a solid tumor. In some embodiments, the cellular proliferative disease is a cancerous cell growth.

In some embodiments, the cancer is selected from sarcoma; lung; bronchus; prostate; breast (including sporadic breast cancers and sufferers of Cowden disease); pancreas; gastrointestinal; colon; rectum; carcinoma; colon carcinoma; adenoma; colorectal adenoma; thyroid; liver; intrahepatic bile duct; hepatocellular; adrenal gland; stomach; gastric, glioma; glioblastoma; endometrial; melanoma; kidney; renal pelvis; urinary bladder; uterine corpus; uterine cervix; vagina; ovary (including clear cell ovarian cancer); multiple myeloma; esophagus; a leukemia; acute myelogenous leukemia; acute megakaryocytic leukemia; chronic myelogenous leukemia; lymphocytic leukemia: myeloid leukemia; T-cell acute lymphoblastic leukemia (T-ALL); B-cell acute lymphoblastic leukemia (B-ALL); acute myeloid leukemia (AML); Chronic Myelomonocytic Leukemia (CMML); T-cell large granular lymphocytic leukemia (T-LGL); T-cell prolymphocytic leukemia (T-PLL), brain; a carcinoma of the brain; oral cavity and pharynx; larynx, small intestine; non-Hodgkin lymphoma; villous colon adenoma; a neoplasia; a neoplasia of epithelial character; lymphoma; a mammary carcinoma; basal cell carcinoma; squamous cell carcinoma; actinic keratosis; neck; head; polycythemia vera; essential thrombocythemia; myelofibrosis with myeloid metaplasia; and Waldenstrom macroglobulinemia.

In some embodiments, the cancer is selected from lung; bronchus; prostate; breast (including sporadic breast cancers and Cowden disease); pancreas; gastrointestinal; colon; rectum; thyroid, liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; a leukemia; acute myelogenous leukemia; chronic myelogenous leukemia; lymphocytic leukemia; myeloid leukemia; brain; oral cavity and pharynx; larynx; small intestine; neck; and head. In some embodiments, the cancer is selected from sarcoma; carcinoma; colon carcinoma; adenoma; colorectal adenoma; glioma; glioblastoma; melanoma: multiple myeloma; a carcinoma of the brain; non-Hodgkin lymphoma; villous colon adenoma; a neoplasia; a neoplasia of epithelial character; lymphoma; a mammary carcinoma; basal cell carcinoma; squamous cell carcinoma; actinic keratosis; polycythemia vera; essential thrombocythemia; myelofibrosis with myeloid metaplasia; and Waldenstrom macroglobulinemia. [0172] In some embodiments, the cancer is selected from lung; bronchus; prostate; breast (including sporadic breast cancers and Cowden disease); pancreas, gastrointestinal; colon; rectum; thyroid; liver; intrahepatic bile duct; hepatocellular; adrenal gland; stomach; gastric; endometrial; kidney; renal pelvis; urinary bladder; uterine corpus; uterine cervix; vagina; ovary (including clear cell ovarian cancer); esophagus; brain; oral cavity and pharynx; larynx; small intestine; neck; and head. In some embodiments, the cancer is a leukemia. In some embodiments, the cancer is acute myelogenous leukemia; chronic myelogenous leukemia; lymphocytic leukemia; or myeloid leukemia.

In some embodiments, the cancer is breast cancer (including sporadic breast cancers and Cowden disease). In some embodiments, the cancer is breast cancer. In some embodiments, the cancer is ER+/HER2− breast cancer. In some embodiments, the cancer is ER+/HER2− breast cancer, and the subject is intolerant to, or ineligible for, treatment with alpelisib. In some embodiments, the cancer is sporadic breast cancer. In some embodiments, the cancer is Cowden disease.

In some embodiments, the cellular proliferative disease has mutant JAK2. In some embodiments, the cellular proliferative disease is a myeloproliferative disorder. In some embodiments, the cancer has mutant JAK2. In some embodiments, the hematopoietic cancer has mutant JAK2 in some embodiments, the myeloproliferative disorder has mutant JAK2.

In some embodiments, the cancer is adenoma; carcinoma; sarcoma; glioma; glioblastoma; melanoma; multiple myeloma; or lymphoma. In some embodiments, the cancer is a colorectal adenoma or avillous colon adenoma. In some embodiments, the cancer is colon carcinoma; a carcinoma of the brain; a mammary carcinoma; basal cell carcinoma; or a squamous cell carcinoma.

In some embodiments, the cancer is a neoplasia or a neoplasia of epithelial character. In some embodiments, the cancer is non-Hodgkin lymphoma. In some embodiments, the cancer is actinic keratosis; polycythemia vera; essential thrombocythemia; myelofibrosis with myeloid metaplasia; or Waldenstrom macroglobulinemia.

In some embodiments, the cellular proliferative disease displays overexpression or amplification of JAK2, or somatic mutation of JAK2.

### Additional Disorders

In some embodiments, the JAK2-mediated disorder is selected from the group consisting of: polycythemia vera, essential thrombocythemia, myelofibrosis with myeloid metaplasia, asthma, COPD, ARDS, PROS (PI3K-related overgrowth syndrome), venous malformation, Loftier's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia greata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In some embodiments, the JAK2-mediated disorder is polycythemia vera, essential thrombocythemia, or myelofibrosis with myeloid metaplasia. In some embodiments, the JAK2-mediated disorder is asthma, COPD, ARDS, PROS (PI3K-related overgrowth syndrome), venous malformation, Loffler's syndrome, eosinophilic pneumonia, parasitic (in particular metazoan) infestation (including tropical eosinophilia), or bronchopulmonary aspergillosis. In some embodiments, the JAK2-mediated disorder is polvarteritis nodosa (including Churg-Strauss syndrome), eosinophilic granuloma, eosinophil-related disorders affecting the airways occasioned by drug-reaction, psoriasis, contact dermatitis, atopic dermatitis, alopecia areata, erythema multiforme, dermatitis herpetiformis, or scleroderma. In some embodiments, the JAK2-mediated disorder is vitiligo, hypersensitivity angiitis, urticaria, bullous pemphigoid, lupus erythematosus, pemphisus, epidermolysis bullosa acquisita, or autoimmune haematogical disorders (e.g. haemolytic anaemia, aplastic anaemia, pure red cell anaemia and idiopathic thrombocytopenia). In some embodiments, the JAK2-mediated disorder is systemic lupus erythematosus, polychondritis, scleroderma, Wegener granulomatosis, dermatomyositis, chronic active hepatitis, myasthenia gravis, Steven-Johnson syndrome, idiopathic sprue, or autoimmune inflammatory bowel disease (e.g. ulcerative colitis and Crohn's disease).

In some embodiments, the JAK2-mediated disorder is endocrine opthalmopathy, Graves' disease, sarcoidosis, alveolitis, chronic hypersensitivity pneumonitis, multiple sclerosis, primary biliary cirrhosis, uveitis (anterior and posterior), interstitial lung fibrosis, or psoriatic arthritis. In some embodiments, the JAK2-mediated disorder is glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, or reperfusion injuries. In some embodiments, the JAK2-mediated disorder is retinopathy, such as diabetic retinopathy or hyperbaric oxygen-induced retinopathy, and conditions characterized by elevated intraocular pressure or secretion of ocular aqueous humor, such as glaucoma.

In some embodiments, the JAK2-mediated disorder is myelofibrosis (MF), polycythemia Vera (PV), essential thrombocythemia (ET), acute megakaryocytic leukemia, T-cell acute lymphoblastic leukemia (T-ALL), B-cell acute lymphoblastic leukemia (B-ALL), acute myeloid leukemia (AML), Chronic Myelomonocytic Leukemia (CMML), T-cell large granular lymphocytic leukemia (T-LGL), T-cell prolymphocytic leukemia (T-PLL), or graft versus host disease (GVHD).

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially nonreactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd. Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), or mass spectrometry, or by chromatography such as high performance liquid chromatography (HPLC) or thin layer chromatography.

The expressions, “ambient temperature,” “room temperature,” and “r.t.” as used herein, are understood in the art, and refer generally to a temperature, e.g., a reaction temperature, that is about the temperature of the room in which the reaction is carried out, for example, a temperature from about 20° C. to about 30° C.

Compounds of the invention can be prepared using numerous preparatory reactions known in the literature. The Schemes below provide general guidance in connection with preparing the compounds of the invention. One skilled in the art would understand that the preparations shown in the Schemes can be modified or optimized using general knowledge of organic chemistry to prepare various compounds of the invention. Example synthetic methods for preparing compounds of the invention are provided in the Schemes below.

The following Examples are provided to illustrate some of the concepts described within this disclosure. While the Examples are considered to provide an embodiment, it should not be considered to limit the more general embodiments described herein.

### EXAMPLES

### General Synthetic Procedures

### Intermediate 1. 4-((3-Bromo-2-methoxyphenyl)amino)-6-(cyclopropanecarboxamido)-N-methylpyridazine-3-carboxamide

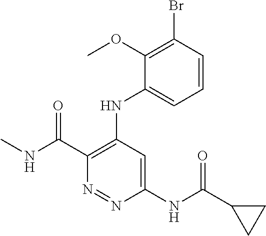 [SMILES: CNC(=O)c1nnc(NC(=O)C2CC2)cc1Nc1cccc(Br)c1OC]

### Step 1. Lithium 4,6-dichloropyridazine-3-carboxylate

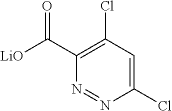

To a solution of methyl 4,6-dichloropyridazine-3-carboxylate (1.00 g, 4.83 mmol) in MeCN (5.0 mL) and water (0.75 mL) was added LiBr (1.26 g, 14.5 mmol) and DIPEA (1.87 g, 14.5 mmol). The reaction was stirred at 25° C. for 3 h. Then the reaction mixture was filtered, and the filter cake was washed with MeCN (5 mL). The filter cake was dried under reduced pressure to afford the title compound (1.00 g, 5.03 mmol, 104% yield) as a white solid, which was used in the next step directly without further purification. ¹H NMR (400 MHz, DMSO-d₆) δ 8.12 (s, 1H).

### Step 2. 4,6-Dichloro-N-methylpyridazine-3-carboxamide

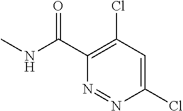

To a solution of lithium 4,6-dichloropyridazine-3-carboxylate (1.00 g, 5.03 mmol) in DCM (15 mL) was added methylamine hydrochloride (815 mg, 12.1 mmol) and pyridine (1.91 g, 24.2 mmol) at 0° C. under N₂. Then to the mixture was added POCl₃ (3.70 g, 24.15 mmol) dropwise at 0° C. The resulting mixture was stirred at 25° C. for 3 h. Then the mixture was poured into water and extracted with DCM (30 mL). The organic layer was washed with a saturated solution of NaHCO₃ (50 mL) and brine (50 mL), dried over Na₂SO₄ and concentrated under reduced pressure to give a residue. The residue was purified by trituration with hexane:EtOAc=10:1 to afford the title compound (720 mg, 3.49 mmol, 72.3% yield) as a white solid. ¹H NMR (400 MHz, DMSO-d₆) δ 9.08-8.81 (m, 1H), 8.96-8.90 (m, 1H), 2.83 (d, J=4.7 Hz, 3H).

### Step 3. 4-((3-Bromo-2-methoxyphenyl)amino)-6-chloro-N-methylpyridazine-3-carboxamide

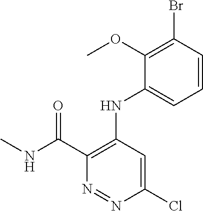 [SMILES: CNC(=O)c1nnc(Cl)cc1Nc1cccc(Br)c1OC]

To a solution of 4,6-dichloro-N-methylpyridazine-3-carboxamide (500 mg, 2.43 mmol) in THF (10 mL) was added 3-bromo-2-methoxyaniline (490 mg, 2.43 mmol). The mixture was purged with N₂ three times and then to the mixture was added lithium hexamethyldisilazide (5.6 mL, 7.28 mmol, 1.3 M in THF) at −10° C. The resulting mixture was stirred at 20° C. for 1 h. The reaction mixture was poured into a saturated solution of NH₄Cl (20 mL). The mixture was extracted with DCM (20 mL×2). The combined organic layer was washed with brine, dried over Na₂SO₄, and concentrated under reduced pressure to give a residue. The residue was purified by trituration with hexane:EtOAc=5:1 to afford the title compound (510 mg, 1.37 mmol, 56.6% yield) as a white solid. LCMS calc. for C₁₃H₁₃BrClN₄O₂ [M+H]⁺: m/z=370.98; Found: 371.1.

### Step 4. 4-((3-Bromo-2-methoxyphenyl)amino)-6-(cyclopropanecarboxamido)-N-methylpyridazine-3-carboxamide

To a solution of 4-(3-bromo-2-methoxyanilino)-6-chloro-N-methylpyridazine-3-carboxamide (500 mg, 1.35 mmol) in 1,4-dioxane (10 mL) and water (2.0 mL) was added cyclopropanecarboxamide (343 mg, 4.04 mmol), Pd₂(dba)₃ (123 mg, 0.13 mmol), dppf (149 mg, 0.27 mmol) and K₃PO₄ (857 mg, 4.04 mmol). The resulting mixture was purged with N₂ three times, and the mixture was stirred at 110° C. for 16 h. The mixture was concentrated to give a residue. The residue was purified by silica gel column chromatography (5-50% EtOAc/hexane) to afford the title compound (210 mg, 0.50 mmol, 37% yield) as a yellow solid. LCMS calc. for C₁₇H₁₉BrN₅O₃ [M+H]⁺: m/z=420.06; Found: 420.1. ¹H NMR (400 MHz, DMSO-d₆) δ 11.35 (s, 1H), 11.02 (s, 1H), 9.18 (d, J=4.9 Hz, 1H), 8.13 (s, 1H), 7.45 (ddd, J=12.6, 8.1, 1.2 Hz, 2H), 7.16 (t, J=8.1 Hz, 1H), 3.73 (s, 3H), 2.85 (dd, J=8.9, 4.8 Hz, 3H), 2.29-1.98 (m, 1H), 0.83 (dd, J=8.5, 4.6 Hz, 4H).

### Intermediate 2. 6-(Cyclopropanecarboxamido)-4-((2-methoxy-3-(4,4,5,5-tetramethyl-1,3,2-dioxaborolan-2-yl)phenyl)amino)-N-methylpyridazine-3-carboxamide

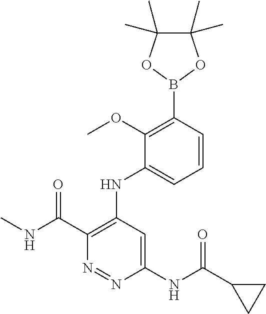 [SMILES: CNC(=O)c1nnc(NC(=O)C2CC2)cc1Nc1cccc(B2OC(C)(C)C(C)(C)O2)c1OC]

To a solution of 4-((3-bromo-2-methoxyphenyl)amino)-6-(cyclopropanecarboxamido)-N-methylpyridazine-3-carboxamide (Int. 1) (1.00 g, 2.38 mmol) in 1,4-dioxane (15.0 mL) was added bis(pinacolato)diboron (1.20 g, 4.73 mmol), Pd(dppf)Cl₂ (174 mg, 0.238 mmol) and KOAc (700 mg, 7.13 mmol). The resulting mixture was purged with N₂ three times, and the mixture was stirred at 100° C. for 16 h. The mixture was diluted with EtOAc (20 mL) and filtered and extracted with EtOAc (50 mL). The organic layer was then washed with brine (100 mL), dried over Na₂SO₄, filtered, and concentrated. The residue was purified by silica gel column chromatography (0-50% EtOAc/hexane) to afford the title compound (660 mg, 1.41 mmol, 59% yield) as a yellow solid. LCMS calc. for C₂₃H₃₁BN₅O₅[M+H]⁺: m/z=468.24; Found: 468.3.

### Intermediate 3. N-(5-Acetyl-4-((3-bromo-2-methoxyphenyl)amino)pyridin-2-yl)cyclopropanecarboxamide

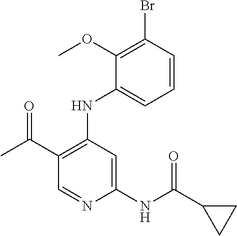 [SMILES: COc1c(Br)cccc1Nc1cc(NC(=O)C2CC2)ncc1C(C)=O]

### Step 1. 1-(4-((3-Bromo-2-methoxyphenyl)amino)-6-chloropyridin-3-yl)ethan-1-one

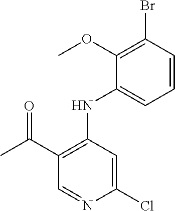 [SMILES: COc1c(Br)cccc1Nc1cc(Cl)ncc1C(C)=O]

To a solution of 1-(4,6-dichloro-3-pyridinyl)ethanone (2.2 g, 11.6 mmol) in ethanol (40 mL) was added 3-bromo-2-methoxyaniline (2.34 g, 11.6 mmol) and HCl (11.6 mL, 139 mmol, 12 M in water) under N₂. The resulting mixture was stirred at 80° C. for 3 hours. The reaction mixture was quenched by adding sat. Na₂CO₃ (aq) (100 mL) and extracted with EtOAc (3×100 mL). The combined organic layers were washed with brine, dried over Na₂SO₄, filtered and concentrated under reduced pressure. The residue obtained was purified by silica gel column chromatography (5-45% EtOAc/Hexanes) to yield the title compound (1.89 g, 46% yield) as a yellow solid. LCMS calc. for C₁₄H₁₃BrClN₂O₂[M+H]⁺: m/z=354.9/356.9; Found 355.0/357.0.

In an alternative preparation, to a solution of 1-(4,6-dichloro-3-pyridinyl)ethanone (4.5 g, 24 mmol) in ethanol (90 mL) was added 3-bromo-2-methoxyaniline (4.8 g, 24 mmol) and HCl (18 mL, 24 mmol) under N₂. The resulting mixture was stirred at 80° C. for 15 h. The reaction mixture was quenched by adding water and extracted with EtOAc (3×50 mL). the combined organic layers were washed with brine, dried over Na₂SO₄, filtered and concentrated under reduced pressure to obtain the crude, which was purified by prep-HPLC on a C₁₈ column (20-70% MeCN in 0.1% TFA(aq), pH=2) to obtain the title compound (4.3 g, 51% yield) as a yellow solid. LCMS calc. for C₁₄H₁₃BrClN₂O₂ [M+H]⁺: m/z=354.9/356.9; Found 355.0/357.0.

### Step 2. N-(5-Acetyl-4-((3-bromo-2-methoxyphenyl)amino)pyridin-2-yl)cyclopropanecarboxamide

To a solution of 1-(4-((3-bromo-2-methoxyphenyl)amino)-6-chloropyridin-3-yl)ethan-1-one (5.0 g, 14 mmol) in 1,4-dioxane (100 mL) and water (10 mL) was added cyclopropanecarboxamide (5.9 g, 70 mmol), dppf (1.6 g, 2.8 mmol), K₃PO₄ (8.9 g, 42 mmol) and Pd₂(dba)₃ (1.3 g, 1.4 mmol) under N₂. The resulting mixture was stirred at 110° C. for 14 hours. The reaction mixture was quenched by adding water (50 mL) and extracted with EtOAc (50 ml×3). The combined organic layers were washed with brine, dried over Na₂SO₄, filtered and concentrated under reduced pressure. The residue obtained was purified by silica gel column chromatography (0-50% EtOAc/Hexanes) to obtain the title compound (5.6 g, 99% yield) as a yellow solid. LCMS calc. for C₁₈H₁₉BrN₃O₃[M+H]⁺: m/z=404.1/406.1; Found 404.1/406.1.

### Intermediates 4-6

Intermediates 4-6 shown below in Table 1 were prepared in accordance with the synthetic protocols set forth in Int. 3 Steps 1-2 using appropriate intermediates, as well as commercial starting materials

TABLE 1
Intermediate 4-6

| Intermediate | Name | Structure | Analytical Data |
|---|---|---|---|
| 4 | N-(5-acetyl-4-((3-bromo- 5-fluoro-2- methoxyphenyl)- amino)pyridin-2-yl)cyclo- propanecarboxamide | 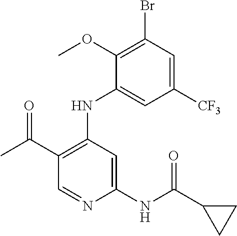 | LCMS calc. for C₁₈H₁₈BrFN₃O₃ [M + H]⁺: m/z = 422.1/424.1; Found: 422.1/424.0 |
| 5 | N-(5-acetyl-4-((3-bromo- 4-fluoro-2- methoxyphenyl)- amino)pyridin-2-yl)cyclo- propanecarboxamide | 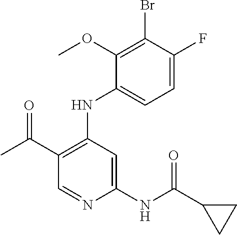 [SMILES: COc1c(Nc2cc(NC(=O)C3CC3)ncc2C(C)=O)ccc(F)c1Br] | LCMS calc. for C₁₈H₁₈BrFN₃O₃ [M + H]⁺: m/z = 422.1/424.1; Found: 422.1/424.1 |
| 6 | N-(5-acetyl-4-((3-bromo- 6-fluoro-2- methoxyphenyl)- amino)pyridin-2-yl)cyclo- propanecarboxamide | 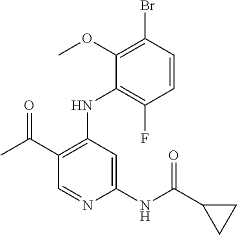 [SMILES: COc1c(Br)ccc(F)c1Nc1cc(NC(=O)C2CC2)ncc1C(C)=O] | LCMS calc. for C₁₈H₁₈BrFN₃O₃ [M + H]⁺: m/z = 422.1/424.1; Found: 421.9/423.9 |

### Intermediate 7. N-(5-Acetyl-4-((3-bromo-2-methoxy-5-(trifluoromethyl)phenyl)-amino)pyridin-2-yl)cyclopropanecarboxamide

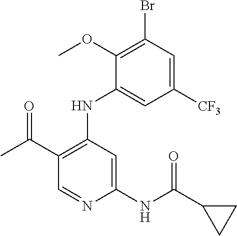 [SMILES: COc1c(Br)cc(C(F)(F)F)cc1Nc1cc(NC(=O)C2CC2)ncc1C(C)=O]

### Step 1. 1-Bromo-2-methoxy-3-nitro-5-(trifluoromethyl)benzene

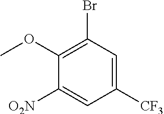 [SMILES: COc1c(Br)cc(C(F)(F)F)cc1[N+](=O)[O-]]

Potassium carbonate (125 mg, 0.9 mmol) and dimethyl sulfate (114 mg, 0.9 mmol) were added in sequence to a stirring solution of 2-bromo-6-nitro-4-(trifluoromethyl)phenol (129 mg, 0.45 mmol) in acetone (5 mL). The reaction mixture was heated to reflux for 2 hours. The product mixture was filtered through celite and concentrated under reduced pressure to yield the title compound (99 mg, 73% yield) which was used without further purification. ¹H NMR (400 MHz, CDCl₃) δ 8.05 (d, J=3.0 Hz, 2H), 4.08 (s, 3H).

### Step 2. 3-Bromo-2-methoxy-5-(trifluoromethyl)aniline

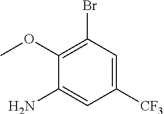

Iron (92 g, 1.65 mmol) and ammonium chloride (176 mg, 3.3 mmol) were added to a stirring solution of 1-bromo-2-methoxy-3-nitro-5-(trifluoromethyl)benzene (99 mg, 0.33 mmol) in ethanol (5 mL) and water (2.5 mL). The reaction mixture was heated to 80° C. for 2 hours. The product mixture was filtered and concentrated under reduced pressure. The residue obtained was diluted with water (30 mL) and extracted with EtOAc (2×30 mL). The combined organic layers were dried over Na₂SO₄, filtered and concentrated under reduced pressure. The residue obtained was purified by silica gel column chromatography (5-30% EtOAc/Hexanes) to yield the title compound (60 mg, 67% yield). LCMS calc. for C₈H₇BrF₃NO [M+H]⁺: m/z=270.0/272.0; Found: 270.0/272.1.

### Step 3. 1-(4-((3-Bromo-2-methoxy-5-(trifluoromethyl)phenyl)amino)-6-chloropyridin-3-yl)ethan-1-one

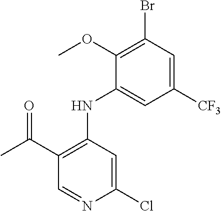 [SMILES: COc1c(Br)cc(C(F)(F)F)cc1Nc1cc(Cl)ncc1C(C)=O]

To a solution of 1-(4,6-dichloro-3-pyridinyl)ethanone (352 mg, 1.85 mmol) in ethanol (5 mL) was added 3-bromo-2-methoxyaniline (500 mg, 1.85 mmol) and HCl (2.5 mL, 60 mmol, 12.1 M in isopropyl acetate) under N₂. The resulting mixture was stirred at 75° C. for 16 h. The reaction mixture was quenched by adding water and was extracted with EtOAc (3×50 mL). The combined organic layers were washed with brine, dried over Na₂SO₄, filtered and concentrated under reduced pressure. The residue obtained was purified by silica gel column chromatography (10-20% EtOAc/Hexanes) to yield the title compound (500 mg, 64% yield). LCMS calc. for C₁₅H₁₂BrClF₃N₂O₂ [M+H]⁺: m/z=423.0/425.0; Found: 423.0/424.9.

### Step 4. N-(5-Acetyl-4-((3-bromo-2-methoxy-5-(trifluoromethyl)phenyl)amino)pyridin-2-yl)cyclopropanecarboxamide

The title compound was prepared in accordance with the synthetic protocols set forth in Int. 3 Step 2 using appropriate intermediates, as well as commercial starting materials. LCMS calc. for C₁₉H₁₈BrF₃N₃O₃ [M+H]⁺: m/z=472.0/474.0; Found: 472.2/474.2.

### Intermediate 8. N-(5-Acetyl-4-((3-bromo-2-methoxy-5-methylphenyl)amino)pyridin-2-yl)cyclopropanecarboxamide

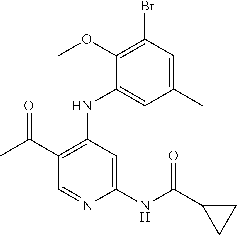 [SMILES: COc1c(Br)cc(C)cc1Nc1cc(NC(=O)C2CC2)ncc1C(C)=O]

### Step 1. 1-Bromo-2-methoxy-5-methyl-3-nitrobenzene

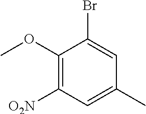

Iodomethane (1.4 mL, 22.6 mmol) was added to a stirring solution of 2-bromo-4-methyl-6-nitrophenol (3.5 g, 15.1 mmol) and potassium carbonate (6.25 g, 45.3 mmol) in DMF (150 mL) at rt. The reaction mixture was heated to 60° C. for 16 hours. The product mixture was diluted with water (100 mL) resulting in precipitation of the product. The suspension was filtered, and the filter cake was dried under reduced pressure to yield the title compound (3.2 g, 86% yield). LCMS calc. for C₈H₉BrNO₃ [M+H]⁺: m/z=246.0/248.0; Found: 246.0/247.0.

### Step 2. N-(5-Acetyl-4-((3-bromo-2-methoxy-5-methylphenyl)amino)pyridin-2-yl)cyclopropanecarboxamide

The title compound was prepared using procedures analogous to Intermediate 7 Steps 2 through 4 using appropriate intermediates and commercial starting materials. LCMS calc. for C₁₉H₂₁BrN₃O₃[M+H]⁺: m/z=418.1/420.1; Found: 418.0/419.9.

### Intermediate 9. (1S,2R)—N-(5-Acetyl-4-((3-bromo-2-methoxyphenyl)amino)pyridin-2-yl)-2-fluorocyclopropane-1-carboxamide

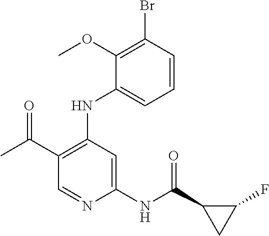 [SMILES: COc1c(Br)cccc1Nc1cc(NC(=O)[C@@H]2C[C@H]2F)ncc1C(C)=O]

### Step 1. tert-Butyl (5-acetyl-4-((3-bromo-2-methoxyphenyl)amino)pyridin-2-yl)carbamate

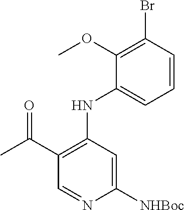 [SMILES: COc1c(Br)cccc1Nc1cc(NC(=O)OC(C)(C)C)ncc1C(C)=O]

To a solution of 1-(4-((3-bromo-2-methoxyphenyl)amino)-6-chloropyridin-3-yl)ethan-1-one (500 mg, 1.4 mmol) in 1,4-dioxane (10 mL) and water (1 mL) was added tert-butyl carbamate (820 mg, 7.0 mmol), dppf (160 mg, 0.28 mmol), K₃PO₄ (900 mg, 4.2 mmol) and Pd₂(dba)₃ (130 mg, 0.14 mmol) under N₂. The resulting mixture was stirred at 110° C. for 12 h. The reaction mixture was quenched by adding water (50 mL) and extracted with EtOAc (50 ml×3). The combined organic layers were washed with brine, dried over Na₂SO₄, filtered and concentrated under reduced pressure. The residue obtained was purified directly by silica gel column chromatography (3:1 hexane; EtOAc;) to obtain the title compound (266 mg, 43% yield). LCMS calc. for C₁₉H₂₃BrN₃O₄[M+H]⁺: m/z=436.1; Found: 436.2.

### Step 2. 1-(6-Amino-4-((3-bromo-2-methoxyphenyl)amino)pyridin-3-yl)ethan-1-one

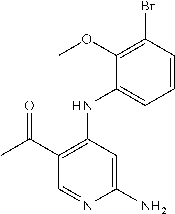 [SMILES: COc1c(Br)cccc1Nc1cc(N)ncc1C(C)=O]

To a solution of tert-butyl (5-acetyl-4-((3-bromo-2-methoxyphenyl)amino)pyridin-2-yl)carbamate (260 mg, 0.60 mmol) in DCM (3 mL) was added trifluoroacetic acid (3 mL, 0.6 mmol) under N₂ at rt. The resulting mixture was stirred for 1 hour. The reaction mixture was concentrated under reduced pressure to obtain the TFA salt of title compound (170 mg, 85% yield). LCMS calc. for C₁₄H₁₅BrN₃O₂[M+H]⁺: m/z=336.1; Found 336.1.

### Step 3. (1S,2R)—N-(5-Acetyl-4-((3-bromo-2-methoxyphenyl)amino)pyridin-2-yl)-2-fluorocyclopropane-1-carboxamide

To a solution of 1-(6-amino-4-((3-bromo-2-methoxyphenyl)amino)pyridin-3-yl)ethan-1-one (30 mg, 0.09 mmol) in pyridine (1.2 mL) was added (1R,2S)-2-fluorocyclopropane-1-carboxylic acid (9.3 mg, 0.09 mmol) under N₂ at rt. Then, POCl₃ (68 mg, 0.45 mmol) was added under N₂ at 0° C. The resulting mixture was stirred at 0° C. for 10 min. The reaction mixture was quenched by adding water (30 mL) and extracted with EtOAc (20 mL×3). The combined organic layers were washed with brine, dried over Na₂SO₄, filtered and concentrated under reduced pressure to obtain the title compound (26 mg, 69% yield). LCMS calc. for C₁₈H₁₈BrFN₃O₃[M+H]⁺: m/z=422.1; Found: 422.2.

### Intermediates 10-15

Intermediates 10-15 shown below in Table 2 were prepared in accordance with the synthetic protocols set forth in Int. 9 Steps 1-3 using appropriate starting materials, as well as commercial starting materials

TABLE 2
Intermediates 10-15

| | | | Analytical |
|---|---|---|---|
| Intermediate | Name | Structure | Data |
| 10 | (1R,2S)-N-(5-acetyl-4-((3- bromo-5-fluoro-2-methoxy- phenyl)amino)pyridin-2-yl)-2- fluorocyclopropane-1- carboxamide | 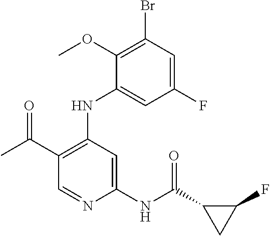 [SMILES: COc1c(Br)cc(F)cc1Nc1cc(NC(=O)[C@H]2C[C@@H]2F)ncc1C(C)=O] | LCMS calc. for C₁₈H₁₇BrF₂N₃O₃ [M + H]⁺: m/z = 440.0/442.0; Found: 440.0/442.0. |
| 11 | (1S,2R)-N-(5-acetyl-4-((3- bromo-5-fluoro-2-methoxy- phenyl)amino)pyridin-2-yl)-2- fluorocyclopropane-1- carboxamide | 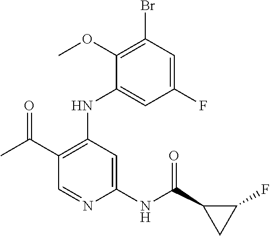 [SMILES: COc1c(Br)cc(F)cc1Nc1cc(NC(=O)[C@@H]2C[C@H]2F)ncc1C(C)=O] | LCMS calc. for C₁₈H₁₇BrF₂N₃O₃ [M + H]⁺: m/z = 440.0/442.0; Found: 440.0/442.0. |
| 12 | (1R,2S)-N-(5-acetyl-4-((3- bromo-2-methoxyphenyl)- amino)pyridin-2-yl)-2- fluorocyclopropane-1- carboxamide | 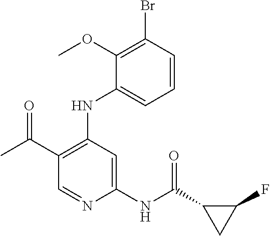 [SMILES: COc1c(Br)cccc1Nc1cc(NC(=O)[C@H]2C[C@@H]2F)ncc1C(C)=O] | LCMS calc. for C₁₈H₁₈BrFN₃O₃ [M + H]⁺: m/z = 422.1/424.1; Found: 422.1/424.1 |
| 13 | (1R,2R)-N-(5-acetyl-4-((3- bromo-2-methoxyphenyl)- amino)pyridin-2-yl)-2- methylcyclopropane-1- carboxamide | 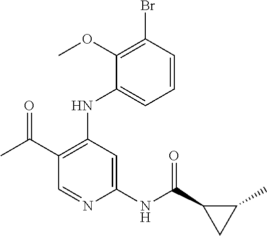 [SMILES: COc1c(Br)cccc1Nc1cc(NC(=O)[C@@H]2C[C@H]2C)ncc1C(C)=O] | LCMS calc. for C₁₉H₂₁BrN₃O₃ [M + H]⁺: m/z = 418.1/420.1; Found: 418.2/420.2 |
| 14 | (1S,2S)-N-(5-acetyl-4-((3- bromo-2-methoxyphenyl)- amino)pyridin-2-yl)-2- methylcyclopropane-1- carboxamide | 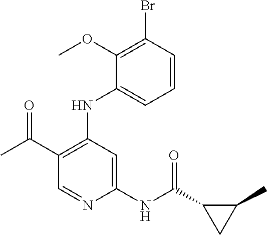 [SMILES: COc1c(Br)cccc1Nc1cc(NC(=O)[C@H]2C[C@@H]2C)ncc1C(C)=O] | LCMS calc. for C₁₉H₂₁BrN₃O₃ [M + H]⁺: m/z = 418.1/420.1; Found: 418.2/420.2. |
| 15 | (1R,2S)-N-(5-acetyl-4-((3- bromo-2-methoxy-5- (trifluoromethyl)phenyl)amino)- pyridin-2-yl)-2- fluorocyclo-propane-1- carboxamide | 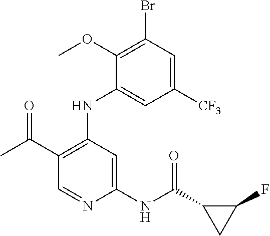 [SMILES: COc1c(Br)cc(C(F)(F)F)cc1Nc1cc(NC(=O)[C@H]2C[C@@H]2F)ncc1C(C)=O] | LCMS calc. for C₁₉H₁₇BrF₄N₃O₃ [M + H]⁺: m/z = 490.0/492.0; Found: 490.0/491.9. |

### Intermediate 16. N-(5-Acetyl-4-((2-chloro-3-methoxypyridin-4-yl)amino)pyridin-2-yl)cyclopropanecarboxamide

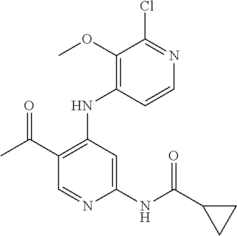 [SMILES: COc1c(Nc2cc(NC(=O)C3CC3)ncc2C(C)=O)ccnc1Cl]

### Step 1. 2-Chloro-4-iodo-3-methoxypyridine

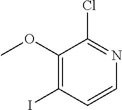

Sodium methoxide (899 μL, 4.86 mmol, 5.4 M in methanol) was added to a stirring solution of 2-chloro-3-fluoro-4-iodopyridine (500 mg, 1.94 mmol) in methanol (3.3 mL) at 0° C. The reaction mixture was heated to 45° C. for 16 hours. The product mixture was diluted with sat. NaHCO₃ (aq) solution (50 mL) and extracted with DCM (3×50 mL). The combined organic layers were dried over MgSO₄, filtered and concentrated under reduced pressure. The residue obtained was purified by silica gel column chromatography (0-50% EtOAc/Hexanes) to yield the title compound (255 mg, 49% yield). LC-MS calc. for C₆H₆ClINO [M+H]⁺: m/z=269.9; Found: 269.9.

### Step 2. 1-(6-Chloro-4-((2,4-dimethoxybenzyl)amino)pyridin-3-yl)ethan-1-one

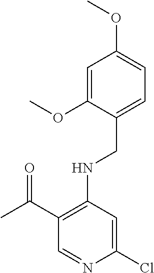 [SMILES: COc1ccc(CNc2cc(Cl)ncc2C(C)=O)c(OC)c1]

Triethylamine (440 μL, 3.2 mmol) and 2,4-dimethoxybenzylamine (356 μL, 2.4 mmol) were added in sequence to a stirring solution of 1-(4,6-dichloro-3-pyridinyl)ethanone (300 mg, 1.6 mmol) in MeCN (4 mL) at rt. The reaction mixture was stirred for 16 hours. The resulting slurry was filtered, and the solid was washed with MeCN (2×2 mL). The solid was dried under reduced pressure to yield the title compound (506 mg, 100% yield). LC-MS calc. for C₁₆H₁₈ClN₂O₃[M+H]⁺: m/z=321.1; Found: 321.1.

### Step 3. N-(5-Acetyl-4-((2,4-dimethoxybenzyl)amino)pyridin-2-yl)cyclopropanecarboxamide

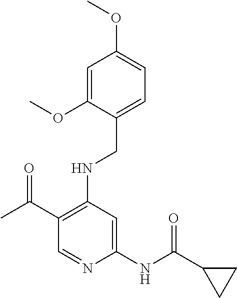 [SMILES: COc1ccc(CNc2cc(NC(=O)C3CC3)ncc2C(C)=O)c(OC)c1]

The title compound was prepared in accordance with the synthetic protocols set forth in Int. 3 Step 2 using appropriate intermediates, as well as commercial starting materials. LC-MS calc. for C₂₀H₂₄N₃O₄ [M+H]⁺: m/z=370.2; Found: 370.2.

### Step 4. N-(5-Acetyl-4-aminopyridin-2-yl)cyclopropanecarboxamide

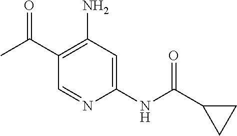 [SMILES: CC(=O)c1cnc(NC(=O)C2CC2)cc1N]

Trifluoroacetic acid (600 μL) was added to a stirring solution of N-(5-acetyl-4-((2,4-dimethoxybenzyl)amino)pyridin-2-yl)cyclopropanecarboxamide (222 mg, 0.6 mmol) in DCM (8 mL) at rt. The reaction mixture was stirred for 2 hours. The product mixture was diluted with sat. NaHCO₃ (aq) solution and extracted with a 3:1 CHCl₃/iPrOH mixture (3×100 mL). The combined organic layers were dried over MgSO₄, filtered and concentrated under reduced pressure to yield the title compound (130 mg, 99% yield) which was used without further purification. LC-MS calc. for C₁₁H₁₄N₃O₂ [M+H]⁺: m/z=220.1; Found: 220.1.

### Step 5. N-(5-Acetyl-4-((2-chloro-3-methoxypyridin-4-yl)amino)pyridin-2-yl)cyclopropanecarboxamide

A 20 mL scintillation vial was charged with N-(5-acetyl-4-aminopyridin-2-yl)cyclopropanecarboxamide (3.54 g, 16.1 mmol), 2-chloro-4-iodo-3-methoxypyridine (4.35 g, 16.1 mmol), tris(dibenzylideneacetone)dipalladium (0) (2.96 g, 3.2 mmol), 4,5-bis(diphenylphosphino)-9,9-dimethylxanthene (1.87 g, 3.2 mmol), and cesium carbonate (15.8 g, 48.4 mmol). The mixture was dissolved in 1,4-dioxane (71 mL), was sparged with nitrogen gas for 5 minutes and sealed. The reaction mixture was heated to 100° C. for 3 hours. The product mixture was diluted with water (100 mL) and extracted with EtOAc (3×100 mL). The combined organic layers were dried over Na₂SO₄, filtered and concentrated under reduced pressure. The residue obtained was purified by silica gel column chromatography (0-100% EtOAc/Hexanes) to yield the title compound (1.79 g, 31% yield). LC-MS calc. for C₁₇H₁₈ClN₄O₃[M+H]⁺: m/z=361.1; Found: 361.0.

### Intermediate 17. (1S,2R)—N-(5-Acetyl-4-((2-chloro-3-methoxypyridin-4-yl)amino)pyridin-2-yl)-2-fluorocyclopropane-1-carboxamide

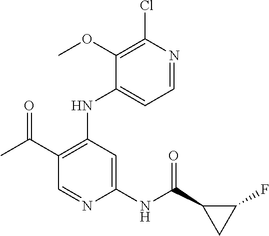 [SMILES: COc1c(Nc2cc(NC(=O)[C@@H]3C[C@H]3F)ncc2C(C)=O)ccnc1Cl]

### Step 1. 1-(6-Amino-4-((2-chloro-3-methoxypyridin-4-yl)amino)pyridin-3-yl)ethan-1-one

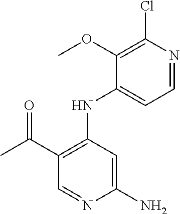 [SMILES: COc1c(Nc2cc(N)ncc2C(C)=O)ccnc1Cl]

Sodium hydroxide (450 μL, 22.2 mmol, 4 M in water) was added to a stirring solution of N-(5-acetyl-4-((2-chloro-3-methoxypyridin-4-yl)amino)pyridin-2-yl)cyclopropanecarboxamide (Int. 16) (800 mg, 2.22 mmol) in 1,4-dioxane (4.5 mL) and water (500 μL). The reaction mixture was heated to 100° C. for 24 hours. The product mixture was diluted with sat. NaHCO₃ (aq) solution (20 mL) and extracted with a 10:1 DCM/MeOH mixture (3×30 mL). The combined organic layers were dried over Na₂SO₄, filtered and concentrated under reduced pressure to yield the title compound (431 mg, 66% yield) which was used without further purification. LCMS calc. for C₁₃H₁₄ClN₄O₂ [M+H]⁺: m/z=293.1; Found: 293.2.

### Step 2. (1S,2R)—N-(5-Acetyl-4-((2-chloro-3-methoxypyridin-4-yl)amino)pyridin-2-yl)-2-fluorocyclopropane-1-carboxamide

Phosphorus (V) oxychloride (1.11 mL, 12 mmol) was added to a stirring solution of 1-(6-amino-4-((2-chloro-3-methoxypyridin-4-yl)amino)pyridin-3-yl)ethan-1-one (700 mg, 2.39 mmol) and (1S,2R)-2-fluorocyclopropane-1-carboxylic acid (249 mg, 2.39 mmol) in pyridine (10 mL) at 0° C. The reaction mixture was stirred for 20 minutes. The product mixture was diluted with water (200 mL) and extracted with EtOAc (3×100 mL). The combined organic layers were dried over Na₂SO₄, filtered and concentrated under reduced pressure. The residue obtained was purified by silica gel column chromatography (0-66% EtOAc/Hexanes) to yield the title compound (672 mg, 74% yield). LCMS calc. for C₁₇H₁₇C₁FN₄O₃[M+H]⁺: m/z=379.1; Found: 379.2.

### Intermediate 18. N-(5-Acetyl-4-chloropyridin-2-yl)cyclopropanecarboxamide

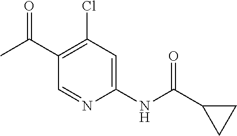 [SMILES: CC(=O)c1cnc(NC(=O)C2CC2)cc1Cl]

A 40 mL scintillation vial was charged with 1-(4,6-dichloropyridin-3-yl)ethan-1-one (300 mg, 1.6 mmol), cyclopropanecarboxamide (134 mg, 1.6 mmol), tris(dibenzylideneacetone)-dipalladium (0) (72 mg, 0.079 mmol), 4,5-bis(diphenylphosphino)-9,9-dimethylxanthene (91 mg, 0.16 mmol), and cesium acetate (909 mg, 4.7 mmol). The reaction mixture was dissolved in 1,4-dioxane (5 mL), sparged with nitrogen gas for 5 minutes, and sealed. The reaction mixture was heated to 85° C. for 2 hours. The product mixture was diluted with water (60 mL) and extracted with DCM (3×60 mL). The combined organic layers were dried over Na₂SO₄, filtered and concentrated under reduced pressure. The residue obtained was purified by silica gel column chromatography (0-5% MeOH/DCM) to yield the title compound (340 mg, 90% yield). LC-MS calc. for C₁₁H₁₂ClN₂O₂ [M+H]⁺: m/z=239.1; Found: 239.0.

### Intermediate 19. (1R,2S)—N-(5-Acetyl-4-chloropyridin-2-yl)-2-fluorocyclopropane-1-carboxamide

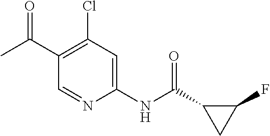 [SMILES: CC(=O)c1cnc(NC(=O)[C@H]2C[C@@H]2F)cc1Cl]

### Step 1. tert-Butyl (5-acetyl-4-chloropyridin-2-yl)carbamate

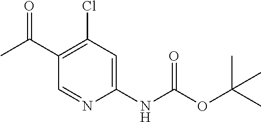 [SMILES: CC(=O)c1cnc(NC(=O)OC(C)(C)C)cc1Cl]

A 40 mL scintillation vial was charged with 1-(4,6-dichloropyridin-3-yl)ethan-1-one (500 mg, 2.6 mmol), tert-butyl carbamate (308 mg, 2.6 mmol), tris(dibenzylideneacetone)dipalladium (0) (120 mg, 0.13 mmol), 4,5-bis(diphenylphosphino)-9,9-dimethylxanthene (152 mg, 0.26 mmol), and cesium acetate (1.52 g, 7.9 mmol). The reaction mixture was dissolved in 1,4-dioxane (8.7 mL), sparged with nitrogen gas for 5 minutes, and sealed. The reaction mixture was heated to 85° C. for 2 hours. The product mixture was diluted with water (60 mL) and extracted with DCM (3×60 mL).

The combined organic layers were dried over Na₂SO₄, filtered and concentrated under reduced pressure. The residue obtained was purified by silica gel column chromatography (0-100% EtOAc/Hexane) to yield the title compound (548 mg, 77% yield). LC-MS calc. for C₈H₈ClN₂O₃ [M+H—C₄H₈]⁺: m/z=215.0; Found: 215.1.

### Step 2. 1-(6-Amino-4-chloropyridin-3-yl)ethan-1-one

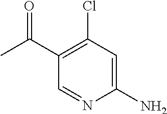

Hydrochloric acid (7.6 mL, 30.4 mmol, 4.0 M in 1,4-dioxane) was added to a stirring solution of tert-butyl (5-acetyl-4-chloropyridin-2-yl)carbamate (548 mg, 2.0 mmol) in DCM (10 mL). The reaction mixture was stirred at rt for 24 hours. The product mixture was diluted with sat. Na₂CO₃ (aq) (60 mL) and was extracted with a 3:1 CHCl₃/iPrOH mixture (3×60 mL). The combined organic layers were dried over MgSO₄, filtered, and concentrated under reduced pressure to yield the title compound (305 mg, 88% yield). LC-MS calc. for C₇H₈ClN₂O [M+H]⁺: m/z=171.0; Found: 171.0.

### Step 3. (1R,2S)—N-(5-Acetyl-4-chloropyridin-2-yl)-2-fluorocyclopropane-1-carboxamide

Phosphorus (V) oxychloride (100 μL, 1.1 mmol) was added dropwise to a stirring solution of 1-(6-amino-4-chloropyridin-3-yl)ethan-1-one (76 mg, 0.45 mmol) and (1R,2S)-2-fluorocyclopropane-1-carboxylic acid (53 mg, 0.51 mmol) in pyridine (3 mL) at 0° C. The reaction mixture was stirred for 30 minutes. The product mixture was quenched with sat. Na₂CO₃ (aq) (20 mL) and extracted with a 3:1 CHCl₃/iPrOH mixture (3×20 mL). The combined organic layers were dried over MgSO₄, filtered and concentrated under reduced pressure. The residue obtained was purified by silica gel column chromatography (0-5% MeOH/DCM) to yield the title compound (90 mg, 79% yield). LC-MS calc. for C₁₁H₁₁C₁FN₂O₂[M+H]⁺: m/z=257.0; Found: 257.0.

### Intermediates 20-22

Intermediates 20-22 shown below in Table 3 were prepared in accordance with the synthetic protocols set forth in Int. 19 Step 3 using appropriate intermediates, as well as commercial starting materials.

TABLE 3
Intermediates 20-22

| Intermediate | Name | Structure | LCMS Data |
|---|---|---|---|
| 20 | (1S,2R)-N-(5-acetyl- 4-chloropyridin-2- yl)-2-fluorocyclo- propane-1- carboxamide | 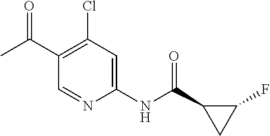 [SMILES: CC(=O)c1cnc(NC(=O)[C@@H]2C[C@H]2F)cc1Cl] | LC-MS calc. for C₁₁H₁₁ClFN₂O₂ [M + H]⁺: m/z = 257.0; Found: 257.0 |
| 21 | (1R,2R)-N-(5-acetyl- 4-chloropyridin-2- yl)-2-methylcyclo- propane-1- carboxamide | 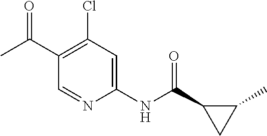 [SMILES: CC(=O)c1cnc(NC(=O)[C@@H]2C[C@H]2C)cc1Cl] | LC-MS calc. for C₁₂H₁₄ClN₂O₂ [M + H]⁺: m/z = 253.1; Found: 253.1 |
| 22 | (1S,2S)-N-(5-acetyl- 4-chloropyridin-2- yl)-2-methylcyclo- propane-1- carboxamide | 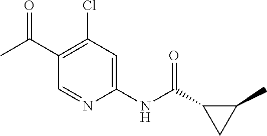 [SMILES: CC(=O)c1cnc(NC(=O)[C@H]2C[C@@H]2C)cc1Cl] | LC-MS calc. for C₁₂H₁₄ClN₂O₂ [M + H]⁺: m/z = 253.1; Found: 253.1 |

### Intermediate 23. 6-(Bromomethyl)-N,N-dimethylpicolinamide

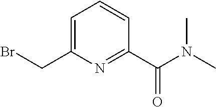

### Step 1. 6-(Hydroxymethyl)-N,N-dimethylpicolinamide

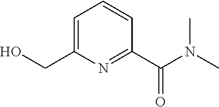

To a solution of methyl 6-(hydroxymethyl)picolinate (2.0 g, 12.0 mmol) in THF (22 mL) was added MgCl₂ (1.7 g, 17.9 mmol) and dimethylamine (20 mL, 40 mmol, 2 M in THF) in sequence. The rection mixture was stirred at 70° C. for 24 hours. The product mixture was diluted with sat. Na₂CO₃ (aq) (100 mL) and extracted with a 3:1 CHCl₃/iPrOH mixture (5×150 mL). The combined organic layers were dried over MgSO₄, filtered and concentrated under reduced pressure to yield the title compound (2.1 g, 97% yield) which was used without further purification. LCMS calc. for C₉H₁₃N₂O₂ [M+H]⁺: m/z=181.1; Found: 181.1.

In an alternative procedure, to a solution of methyl 6-(hydroxymethyl)pyridine-2-carboxylate (2.0 g, 12.0 mmol) in THF (50 mL) was added MgCl₂ (3.42 g, 35.9 mmol) and dimethylamine (15.0 mL, 29.9 mmol, 2M in THF). The rection mixture was stirred at 65° C. for 18 h then cooled and extracted with DCM, which was then dried over Na₂SO₄, condensed, and used directly in the next step. LCMS calcd. for C₉H₁₃N₂O₂ [M+H]⁺: m/z=181.1; Found: 181.1.

### Step 2. 6-(Bromomethyl)-N,N-dimethylpicolinamide

Phosphorus tribromide (1.76 mL, 18.8 mmol) was added to a stirring solution of 6-(hydroxymethyl)-N,N-dimethylpicolinamide (3.04 g, 16.9 mmol) in chloroform (45 mL) at 0° C. The reaction mixture warmed to rt and stirred for 1.5 hours. The product mixture was then cooled to 0° C., diluted with sat. K₂CO₃ (aq) (100 mL) and extracted with DCM (3×80 mL). The combined organic layers were dried over Na₂SO₄, filtered, and concentrated under reduced pressure. The residue obtained was purified by silica gel column chromatography (0-100% EtOAc/Hexanes) to yield the title compound (2.9 g, 71% yield). LCMS calc. for C₉H₁₂BrN₂O [M+H]⁺: m/z=243.0/245.0; Found: 242.9/244.9.

### Intermediate 24. 6-Formyl-N,N-dimethylpicolinamide

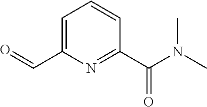

### Step 1. 6-Formyl-N,N-dimethylpicolinamide

2-Iodoxybenzoic acid (6.66 g, 16.6 mmol) was added to a stirring solution of 6-(hydroxymethyl)-N,N-dimethylpicolinamide (2.0 g, 11 mmol) in MeCN (25 mL). The reaction mixture was heated to 55° C. and stirred for 2 hours. The product mixture was diluted with water (150 mL) and extracted with EtOAc (3×100 mL). The combined organic layers were dried over Na₂SO₄, filtered and concentrated under reduced pressure. The residue obtained was purified by silica gel column chromatography (0-100% EtOAc/Hexane) to yield the title compound (1.75 g, 88% yield). LC-MS calc. for C₉H₁₁N₂O₂ [M+H]⁺: m/z=179.1; Found: 179.1.

### Intermediate 25. 8-Bromo-1,3,4,5-tetrahydro-2H-benzo[e][1,4]diazepin-2-one

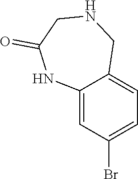 [SMILES: O=C1CNCc2ccc(Br)cc2N1]

### Step 1. tert-Butyl (2-amino-4-bromobenzyl)glycinate

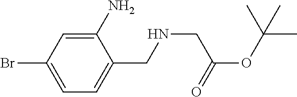 [SMILES: CC(C)(C)OC(=O)CNCc1ccc(Br)cc1N]

2-Amino-4-bromobenzaldehyde (30 mg, 0.15 mmol) and tert-butyl glycinate (110 mg, 0.83 mmol) were dissolved in DMSO (1.5 mL) and stirred at rt for an hour followed by the addition of sodium triacetoxyborohydride (180 mg, 0.83 mmol) in one portion. The reaction mixture was stirred overnight at rt. After completion, it was diluted with MeOH and purified by prep HPLC (0.1% formic acid in water and MeCN). The pure fractions were concentrated, basified with NaHCO₃ (aq) and extracted with DCM. The combined organic layers were dried over Na₂SO₄, filtered, and concentrated under reduced pressure to obtain the title compound (47 mg, 99% yield). LCMS calc. for C₁₃H₂₀BrN₂O₂[M+H]⁺: m/z=315.1; Found: 315.1.

### Step 2. 8-Bromo-1,3,4,5-tetrahydro-2H-benzo[e][1,4]diazepin-2-one

To tert-butyl (2-amino-4-bromobenzyl)glycinate (2.7 g, 8.5 mmol) in DCM (6 mL) was added trifluoroacetic acid (6 mL, 78 mmol) dropwise. After overnight, the reaction was concentrated under reduced pressure. The residue obtained was dissolved in toluene (20 mL) and the reaction was concentrated again to dryness under reduced pressure (repeated 2×). The resulting residue was dissolved in anhydrous toluene (20 mL) and refluxed at 120° C. After 2 hours, the reaction was complete and cooled to rt. The crude was purified by FCC (0-25% MeOH:DCM) to yield the title compound (2.1 g, quant. yield). ¹H NMR (300 MHz, MeOD) δ 7.46 (dd, J=8.1, 1.8 Hz, 1H), 7.42 (s, 1H), 7.38 (t, J=2.4 Hz, 1H), 4.29 (s, 2H), 3.74 (s, 2H).

### Intermediate 26. 6-((8-Bromo-2-oxo-1,2,3,5-tetrahydro-4H-benzo[e][1,4]diazepin-4-yl)methyl)-N,N-dimethylpicolinamide

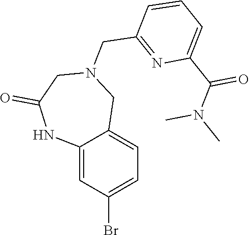 [SMILES: CN(C)C(=O)c1cccc(CN2CC(=O)Nc3cc(Br)ccc3C2)n1]

A mixture of 8-bromo-1,3,4,5-tetrahydro-2H-benzo[e][1,4]diazepin-2-one (520 mg, 2.2 mmol) and 6-formyl-N,N-dimethylpicolinamide (Int. 24) (380 mg, 2.2 mmol) in DCM (15 mL) was stirred at rt for 1 hour, followed by the addition of sodium triacetoxyborohydride (1.4 g, 6.5 mmol). After stirring overnight, the reaction was poured into sat. NaHCO₃ (aq) and extracted 3×(DCM/MeOH (v/v=15/1)). The combined organic phase was washed with brine and dried over Na₂SO₄, filtered and condensed. The crude was purified by FCC (0-10% MeOH/DCM) to yield the title compound (610 mg, 70% yield). LCMS calc. for C₁₈H₂₀BrN₄O₂[M+H]⁺: m/z=403.1; Found: 403.1.

### Intermediate 27. (S)-6-((8-Bromo-2-oxo-1-(tetrahydrofuran-3-yl)-1,2,3,5-tetrahydro-4H-benzo[e][1,4]diazepin-4-yl)methyl)-N,N-dimethylpicolinamide

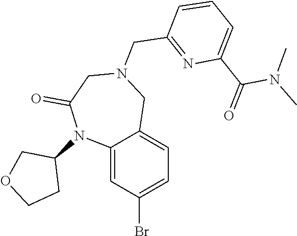 [SMILES: CN(C)C(=O)c1cccc(CN2CC(=O)N([C@H]3CCOC3)c3cc(Br)ccc3C2)n1]

Cesium carbonate (4.85 g, 14.9 mmol) was added to a stirring solution of 6-((8-bromo-2-oxo-1,2,3,5-tetrahydro-4H-benzo[e][1,4]diazepin-4-yl)methyl)-N,N-dimethylpicolinamide (3.00 g, 7.4 mmol) and 3-iodotetrahydrofuran (2.21 g, 11.2 mmol) in DMF (150 mL) and the resulting reaction mixture was sealed and stirred at 80° C. for 2 days. The cooled reaction mixture was then poured into water and extracted with EtOAc. The combined extracts were washed with brine, dried, condensed, and purified with a DAICEL CHIRAL PAK IC column (250 mm×30 mm, 10 μM) by using 35% CO₂/(20% MeCN/iPrOH) with a flow rate of 100 mL/min. to afford the title compound (669 mg, 19% yield). LC-MS calc. for C₂₂H₂₆BrN₄O₃[M+H]⁺: m/z=473.1/475.1. Found: 473.1/475.0.

### Intermediate 28. 6-((8-Bromo-2-oxo-1,2,3,5-tetrahydro-4H-pyrido[3,2-e][1,4]diazepin-4-yl)methyl)-N,N-dimethylpicolinamide

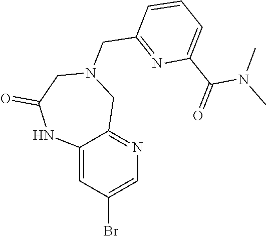 [SMILES: CN(C)C(=O)c1cccc(CN2CC(=O)Nc3cc(Br)cnc3C2)n1]

### Step 1. tert-Butyl ((3-amino-5-bromopyridin-2-yl)methyl)glycinate

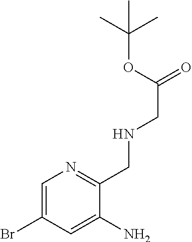 [SMILES: CC(C)(C)OC(=O)CNCc1ncc(Br)cc1N]

Borane dimethyl sulfide complex (4.3 mL, 45.5 mmol) was added dropwise to a stirring solution of 3-amino-5-bromopicolinonitrile (3.0 g, 15.2 mmol) in THF (36 mL). The reaction mixture was heated to 50° C. and stirred for 1 hour. The product mixture was cooled to 0° C. and quenched with dropwise addition of methanol (10 mL). The diluted product mixture was concentrated under reduced pressure. The residue obtained was dissolved in THF (36 mL). The mixture was cooled to 0° C. and triethylamine (6.3 mL, 45.5 mmol) and tert-butyl 2-bromoacetate (2.13 mL, 14.4 mmol) were added sequentially. The reaction mixture was stirred overnight and allowed to warm to rt. The product mixture was diluted with EtOAc (100 mL), washed with brine (100 mL), dried over Na₂SO₄, filtered and concentrated under reduced pressure. The residue obtained was purified by silica gel column chromatography (0-50% EtOAc/Hexanes) to yield the title compound (1.55 g, 32% yield). LC-MS calc. for C₁₂H₁₉BrN₃O₂[M+H]⁺: m/z=316.1/318.1; Found: 316.0/318.0.

### Step 2. tert-Butyl N-((3-amino-5-bromopyridin-2-yl)methyl)-N-((6-(dimethylcarbamoyl)-pyridin-2-yl)methyl)glycinate

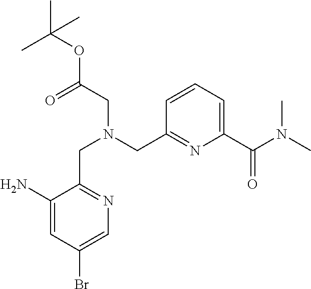 [SMILES: CN(C)C(=O)c1cccc(CN(CC(=O)OC(C)(C)C)Cc2ncc(Br)cc2N)n1]

Potassium carbonate (2.03 g, 14.7 mmol) was added to a stirring solution of tert-butyl ((3-amino-5-bromopyridin-2-yl)methyl)glycinate (1.55 g, 4.9 mmol) and 6-(bromomethyl)-N,N-dimethylpicolinamide (Int. 23) (1.19 g, 4.9 mmol) in MeCN (16 mL). The reaction mixture was heated to 50° C. and stirred for 3 hours. The product mixture was diluted with EtOAc (80 mL). The diluted product mixture was washed with brine, dried over Na₂SO₄, filtered and concentrated under reduced pressure. The residue obtained was purified by silica gel column chromatography (0-10% MeOH/DCM) to yield the title compound (1.93 g, 82% yield). LC-MS calc. for C₂₁H₂₉BrN₅O₃ [M+H]⁺: m/z=478.1/480.1; Found: 478.1/480.1.

### Step 3. 6-((8-Bromo-2-oxo-1,2,3,5-tetrahydro-4H-pyrido[3,2-e][1,4]diazepin-4-yl)methyl)-N,N-dimethylpicolinamide

Sodium hydride (303 mg, 7.6 mmol, 60% dispersion in mineral oil) was added to a stirring solution of tert-butyl N-((3-amino-5-bromopyridin-2-yl)methyl)-N-((6-(dimethyl-carbamoyl)pyridin-2-yl)methyl)glycinate (1.21 g, 2.53 mmol) in THF (24 mL). The reaction mixture was heated to 60° C. and stirred for 3 hours. The product mixture was diluted with sat. NaHCO₃ (aq) solution (100 mL) and extracted with a 3:1 CHCl₃/iPrOH mixture (3×100 mL). The combined organic layers were dried over MgSO₄, filtered and concentrated under reduced pressure. The residue obtained was purified by silica gel column chromatography (0-10% MeOH/DCM) to yield the title compound (1.0 g, 98% yield). LC-MS calc. for C₁₇H₁₉BrN₅O₂[M+H]⁺: m/z=404.1/406.1; Found: 404.1/406.1.

### Intermediate 29-30

Intermediates 29-30 shown below in Table 4 were prepared in accordance with the synthetic protocols set forth in Int. 28 Steps 1-3 using appropriate intermediates, as well as commercial starting materials.

TABLE 4
Intermediate 29-30

| Inter- | | | |
|---|---|---|---|
| mediate | Name | Structure | Analytical Data |
| 29 | 6-((8-bromo-6-fluoro-2- oxo-1,2,3,5-tetrahydro-4H- benzo[e][1,4]diazepin-4- yl)methyl)-N,N- dimethylpicolinamide | 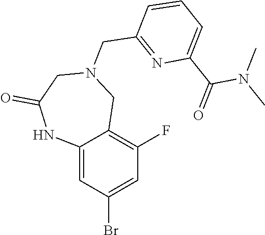 [SMILES: CN(C)C(=O)c1cccc(CN2CC(=O)Nc3cc(Br)cc(F)c3C2)n1] | LCMS calc. for C₁₈H₁₉BrFN₄O₂ [M + H]⁺: m/z = 421.1/423.1; Found: 421.1/423.1. |
| 30 | 6-((8-bromo-7-fluoro-2- oxo-1,2,3,5-tetrahydro-4H- benzo[e][1,4]diazepin-4- yl)methyl)-N,N- dimethylpicolinamide | 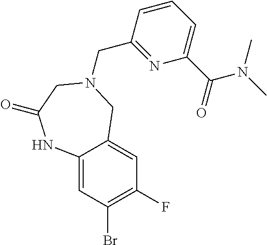 [SMILES: CN(C)C(=O)c1cccc(CN2CC(=O)Nc3cc(Br)c(F)cc3C2)n1] | LCMS calc. for C₁₈H₁₉BrFN₄O₂ [M + H]⁺: m/z = 421.1/423.1; Found: 421.1/423.1. |

### Intermediate 31. 6-((8-Chloro-2-oxo-2,3-dihydro-1H-pyrido[4,3-e][1,4]diazepin-4(5H)-yl)methyl)-N,N-dimethylpicolinamide

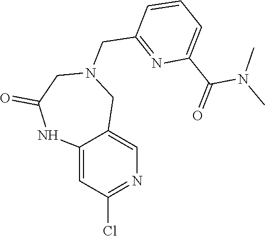 [SMILES: CN(C)C(=O)c1cccc(CN2CC(=O)Nc3cc(Cl)ncc3C2)n1]

### Step 1. tert-Butyl ((4-amino-6-chloropyridin-3-yl)methyl)glycinate

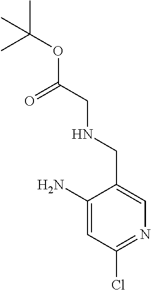 [SMILES: CC(C)(C)OC(=O)CNCc1cnc(Cl)cc1N]

To a solution of 4-amino-6-chloronicotinaldehyde (391 mg, 2.5 mmol) and tert-butyl glycinate (984 mg, 7.5 mmol) in DMSO (10 mL) was added sodium triacetoxyborohydride (2.65 g, 12.5 mmol). The rection mixture was stirred at 25° C. overnight. The product mixture was diluted with water and extracted with DCM. The organic layer was washed with brine, dried over Na₂SO₄, filtered and concentrated under reduced pressure. The residue obtained was purified by silica gel chromatography (0-10% MeOH/DCM) to give the title compound (600 mg, 88% yield). LCMS calc. for C₁₂H₁₉ClN₃O₂[M+H]⁺: m/z=272.1; Found: 272.2.

### Step 2. tert-Butyl N-((4-amino-6-chloropyridin-3-yl)methyl)-N-((6-(dimethylcarbamoyl)-pyridin-2-yl)methyl)glycinate

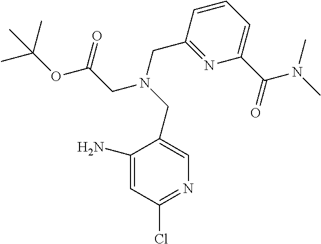 [SMILES: CN(C)C(=O)c1cccc(CN(CC(=O)OC(C)(C)C)Cc2cnc(Cl)cc2N)n1]

To a solution of tert-butyl ((4-amino-6-chloropyridin-3-yl)methyl)glycinate (300 mg, 1.1 mmol) and 6-(bromomethyl)-N,N-dimethylpicolinamide (Int. 23) (295 mg, 1.21 mmol) in acetonitrile (5 mL) was added potassium carbonate (458 mg, 3.31 mmol). The reaction mixture was stirred at 50° C. overnight. The product mixture was diluted with water and was extracted with DCM. The organic layer was washed with brine, dried over Na₂SO₄, filtered and concentrated under reduced pressure. The residue obtained was purified by prep-HPLC (20-70% MeCN in 0.1% TFA (aq), pH=2) to give the title compound (120 mg, 25% yield). LCMS calc. for C₂₁H₂₉ClN₅O₃ [M+H]⁺: m/z=434.19; Found: 434.26. ¹H NMR (400 MHz, Chloroform-d) δ 8.20 (s, 1H), 7.95 (s, 1H), 7.44 (s, 1H), 7.38 (s, 1H), 6.56 (s, 1H), 4.28-4.11 (m, 2H), 3.98 (s, 2H), 3.62 (s, 2H), 3.17 (s, 3H), 2.99 (s, 3H), 1.47 (s, 9H).

### Step 3. N-((4-Amino-6-chloropyridin-3-yl)methyl)-N-((6-(dimethylcarbamoyl)pyridin-2-yl)methyl)glycine

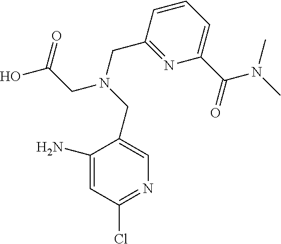 [SMILES: CN(C)C(=O)c1cccc(CN(CC(=O)O)Cc2cnc(Cl)cc2N)n1]

To a solution of tert-butyl N-((4-amino-6-chloropyridin-3-yl)methyl)-N-((6-(dimethyl-carbamoyl)pyridin-2-yl)methyl)glycinate (200 mg, 0.46 mmol) was added trifluoroacetic acid (3.0 mL). The reaction mixture was stirred at 50° C. for two hours. The product mixture was concentrated to dryness to afford the crude product which was used directly in the next step. LCMS calc. for C₁₇H₂₁ClN₅O₃[M+H]⁺: m/z=378.1; Found: 378.1.

### Step 4. 6-((8-Chloro-2-oxo-2,3-dihydro-1H-pyrido[4,3-e][1,4]diazepin-4(5H)-yl)methyl)-N,N-dimethylpicolinamide

To a solution of N-((4-amino-6-chloropyridin-3-yl)methyl)-N-((6-(dimethylcarbamoyl)-pyridin-2-yl)methyl)glycine (55.0 mg, 0.15 mmol) and N,N-diisopropylethylamine (22.6 mg, 0.17 mmol) in DMF (5 mL) was added 2-(7-Azabenzotriazol-1-yl)-N,N,N′,N′-tetramethyluronium hexafluorophosphate (60.9 mg, 0.16 mmol). The reaction mixture was stirred at 15° C. for two hours. The product mixture was diluted with water and extracted with DCM. The organic layer was washed with brine, dried over Na₂SO₄, filtered and concentrated under reduced pressure. The residue was purified by prep-TLC (Ethyl acetate) to give the title compound (25.0 mg, 48% yield) LCMS calc. for C₁₇H₁₉ClN₅O₂[M+H]⁺: m/z=360.1; Found: 360.0. ¹H NMR (400 MHz, Chloroform-d) δ 8.35 (s, 1H), 7.98 (s, 1H), 7.79 (t, J=7.7 Hz, 1H), 7.52 (d, J=7.7 Hz, 1H), 7.41-7.37 (m, 1H), 6.87 (s, 1H), 4.03 (s, 2H), 3.91 (s, 2H), 3.88 (s, 2H), 3.12 (s, 3H), 3.01 (s, 3H).

### Intermediate 32. tert-Butyl 8-bromo-2-oxo-1,2,3,5-tetrahydro-4H-pyrido[3,2-e][1,4]diazepine-4-carboxylate

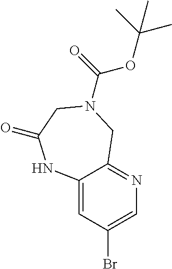 [SMILES: CC(C)(C)OC(=O)N1CC(=O)Nc2cc(Br)cnc2C1]

### Step 1. tert-Butyl N-((3-amino-5-bromopyridin-2-yl)methyl)-N-(tert-butoxycarbonyl)-glycinate

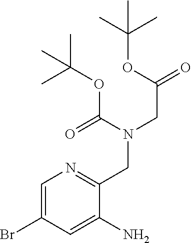 [SMILES: CC(C)(C)OC(=O)CN(Cc1ncc(Br)cc1N)C(=O)OC(C)(C)C]

Di-tert-butyl dicarbonate (959 μL, 4.17 mmol) was added to a stirring solution of tert-butyl ((3-amino-5-bromopyridin-2-yl)methyl)glycinate (1.2 g, 3.8 mmol) and N,N-diisopropylethylamine (991 μL, 5.69 mmol) in DCM (20 mL) at rt. The reaction mixture was stirred for 2 hours. The product mixture was diluted with water (60 mL) and extracted with EtOAc (2×50 mL). The combined organic layers were dried over Na₂SO₄, filtered and concentrated under reduced pressure. The residue obtained was purified by silica gel column chromatography (0-0.3% MeOH/DCM) to yield the title compound (1.58 g, 82% yield). LCMS calc. for C₁₇H₂₇BrN₃O₄ [M+H]⁺: m/z=416.1/418.2; Found: 416.2/418.2.

### Step 2. N-((3-Amino-5-bromopyridin-2-yl)methyl)-N-(tert-butoxycarbonyl)glycine

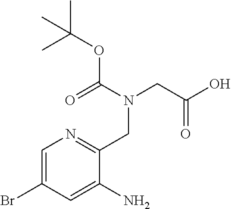 [SMILES: CC(C)(C)OC(=O)N(CC(=O)O)Cc1ncc(Br)cc1N]

Sodium hydroxide (1.2 g, 30 mmol) was added to a stirring solution of tert-butyl N-((3-amino-5-bromopyridin-2-yl)methyl)-N-(tert-butoxycarbonyl)glycinate (2.5 g, 6.0 mmol) in MeOH (8 mL), water (8 mL), and THF (8 mL). The reaction mixture was stirred at rt for 16 hours. The product mixture was concentrated under reduced pressure. The residue obtained was diluted with water (100 mL) and extracted with DCM (3×100 mL). The combined organic layers were dried over Na₂SO₄, filtered and concentrated under reduced pressure to yield the title compound (2.0 g, 92% yield) which was used without further purification. LCMS calc. for C₁₃H₁₉BrN₃O₄[M+H]⁺: m/z=360.1/362.1; Found: 359.7/361.6.

### Step 3. tert-Butyl 8-bromo-2-oxo-1,2,3,5-tetrahydro-4H-pyrido[3,2-e][1,4]diazepine-4-carboxylate

Pyridine (1.12 mL, 13.9 mmol), 1-ethyl-3-(3-dimethylaminopropyl)carbodiimide (639 mg, 3.33 mmol) and 4-dimethylaminopyridine (34 mg, 0.28 mmol) were added in sequence to a stirring solution of N-((3-amino-5-bromopyridin-2-yl)methyl)-N-(tert-butoxycarbonyl)glycine (1.0 g, 2.78 mmol) in 1,2-dichloroethane (15 mL). The reaction mixture was heated to 40° C. and stirred for 1 hour. The product mixture was diluted with water (100 mL) and extracted with EtOAc (3×100 mL). The combined organic layers were dried over Na₂SO₄, filtered, and concentrated under reduced pressure. The residue obtained was purified by silica gel column chromatography (0-50% EtOAc) to yield the title compound (674 mg, 71% yield). LCMS calc. for C₁₃H₁₇BrN₃O₃ [M+H]⁺: m/z=342.0/344.0; Found: 342.2/344.2.

### Intermediate 33. (S)-6-((8-Bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide and (R)-6-((8-bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide

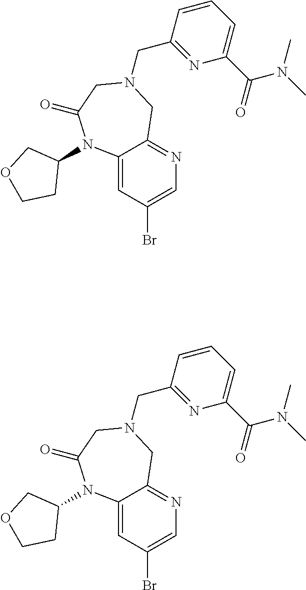 [SMILES: CN(C)C(=O)c1cccc(CN2CC(=O)N([C@H]3CCOC3)c3cc(Br)cnc3C2)n1 ; CN(C)C(=O)c1cccc(CN2CC(=O)N([C@@H]3CCOC3)c3cc(Br)cnc3C2)n1]

### Step 1. (S)-6-((8-Bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide and (R)-6-((8-bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide

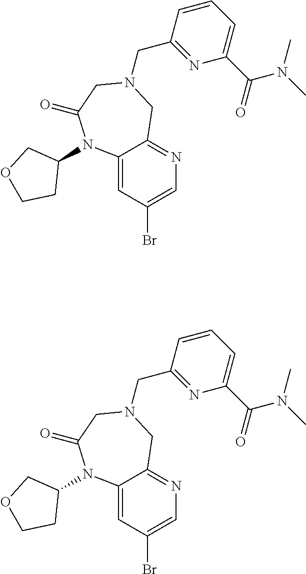 [SMILES: CN(C)C(=O)c1cccc(CN2CC(=O)N([C@H]3CCOC3)c3cc(Br)cnc3C2)n1 ; CN(C)C(=O)c1cccc(CN2CC(=O)N([C@@H]3CCOC3)c3cc(Br)cnc3C2)n1]

(R)-3-Iodotetrahydrofuran (612 mg, 3.1 mmol) was added to a stirring solution of 6-((8-bromo-2-oxo-1,2,3,5-tetrahydro-4H-pyrido[3,2-e][1,4]diazepin-4-yl)methyl)-N,N-dimethylpicolinamide (Int. 28) (250 mg, 0.62 mmol) and cesium carbonate (604 mg, 1.9 mmol) in DMF (3 mL). The reaction mixture was heated to 60° C. for 2 hours. The reaction mixture was cooled to rt, and (R)-3-iodotetrahydrofuran (612 mg, 3.1 mmol) and cesium carbonate (604 mg, 1.9 mmol) were added sequentially to the reaction mixture. The reaction mixture was heated to 60° C. for 2 hours. The product mixture was diluted with brine (60 mL) and extracted with a 3:1 CHCl₃/iPrOH mixture (3×60 mL). The combined organic layers were dried over MgSO₄, filtered and concentrated under reduced pressure. The residue obtained was purified by silica gel column chromatography (0-5% MeOH/DCM) to yield a mixture of the title compounds (169 mg, 58% yield). LC-MS calc. for C₂₁H₂₅BrN₅O₃[M+H]⁺: m/z=474.1/476.1; Found: 474.1/476.1.

### Step 2. (S)-6-((8-Bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide and (R)-6-((8-bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide

The mixture of (S)-6-((8-bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide and (R)-6-((8-bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide was purified via chiral separation (Lux Cellulose-3, 30 mL/min of 70:15:15 Hexanes/IPA/MeOH) to yield (S)-6-((8-bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide (83 mg, Peak A, 49% yield) and (R)-6-((8-bromo-2-oxo-1-(tetrahydrofuran-3-yl)-1,2,3,5-tetrahydro-4H-pyrido[3,2-e][1,4]diazepin-4-yl)methyl)-N,N-dimethylpicolinamide (33 mg, Peak B, 20% yield). LC-MS calc. for C₂₁H₂₅BrN₅O₃[M+H]⁺: m/z=474.1/476.1; Found: 474.1/476.1.

### Intermediate 34. (S)-6-((8-Bromo-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide

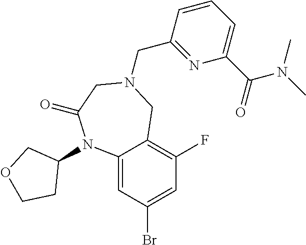 [SMILES: CN(C)C(=O)c1cccc(CN2CC(=O)N([C@H]3CCOC3)c3cc(Br)cc(F)c3C2)n1]

### Step 1. (S)-6-((8-Bromo-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide and (R)-6-((8-bromo-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide

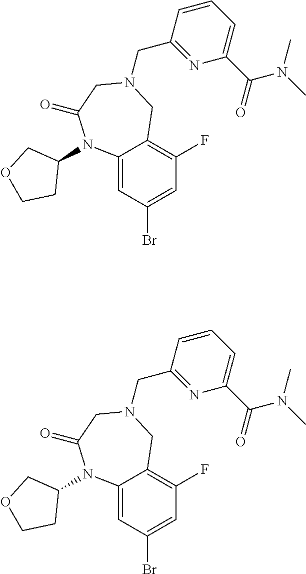 [SMILES: CN(C)C(=O)c1cccc(CN2CC(=O)N([C@H]3CCOC3)c3cc(Br)cc(F)c3C2)n1 ; CN(C)C(=O)c1cccc(CN2CC(=O)N([C@@H]3CCOC3)c3cc(Br)cc(F)c3C2)n1]

The title compounds were prepared using synthetic protocols set forth in Int. 33 Step 1 using appropriate intermediates and commercial starting materials. LCMS calc. for C₂₂H₂₅BrFN₄O₃ [M+H]⁺: m/z=491.1/493.1; Found: 491.1/493.1.

### Step 2. (S)-6-((8-Bromo-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide

The mixture of (S)-6-((8-bromo-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide and (R)-6-((8-bromo-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide was purified via chiral separation (Lux Cellulose-3, 30 mL/min of 70:15:15 Hexanes/IPA/MeOH) to yield (S)-6-((8-bromo-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide (62 mg, Peak A, 55% yield) LCMS calc. for C₂₂H₂₅BrFN₄O₃[M+H]⁺: m/z=491.1/493.1; Found: 491.1/493.1.

### Example 1. 6-(Cyclopropanecarboxamido)-4-((3-(1-ethyl-4-((6-(dimethylcarbamoyl)pyridin-2-yl)methyl)-2-oxo-2,3,4,5-tetrahydro-1H-benzo[e][1,4]diazepin-8-yl)-2-methoxyphenyl) amino)-N-methylpyridazine-3-carboxamide

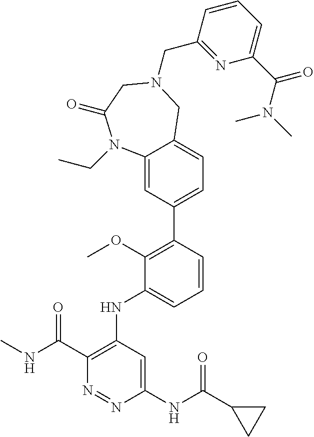 [SMILES: CCN1C(=O)CN(Cc2cccc(C(=O)N(C)C)n2)Cc2ccc(-c3cccc(Nc4cc(NC(=O)C5CC5)nnc4C(=O)NC)c3OC)cc21]

### Step 1. 6-((8-Bromo-1-ethyl-2-oxo-1,2,3,5-tetrahydro-4H-benzo[e][1,4]diazepin-4-yl)methyl)-N,N-dimethylpicolinamide

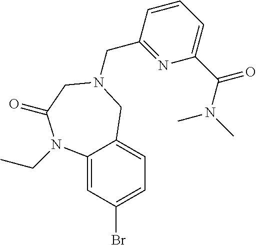 [SMILES: CCN1C(=O)CN(Cc2cccc(C(=O)N(C)C)n2)Cc2ccc(Br)cc21]

6-((8-Bromo-2-oxo-1,2,3,5-tetrahydro-4H-benzo[e][1,4]diazepin-4-yl)methyl)-N,N-dimethylpicolinamide (62 mg, 150 μmol), cesium carbonate (100 mg, 308 μmol) and iodoethane (48 mg, 310 μmol) in DMSO (1 mL) were stirred at rt for 1.5 h. The reaction was partitioned between EtOAc (25 mL) and water (25 mL). The organic phase was separated and further washed with brine (25 mL), dried over Na₂SO₄, filtered and concentrated. The residue was purified by prep-HPLC (15-75% MeCN in H₂O w/0.1% TFA) to yield the title compound as yellow solid (43 mg, 65% yield). LCMS calc. for C₂₀H₂₄BrN₄O₂[M+H]⁺: m/z=431.1; Found: 431.2.

### Step 2. 6-(Cyclopropanecarboxamido)-4-((3-(1-ethyl-4-((6-(methylcarbamoyl)pyridin-2-yl)methyl)-2-oxo-2,3,4,5-tetrahydro-1H-benzo[e][1,4]diazepin-8-yl)-2-methoxyphenyl)amino)-N-methylpyridazine-3-carboxamide

6-((8-Bromo-1-ethyl-2-oxo-1,2,3,5-tetrahydro-4H-benzo[e][1,4]diazepin-4-yl)methyl)-N,N-dimethylpicolinamide (20 mg, 46 μmol), 6-(cyclopropanecarboxamido)-4-((2-methoxy-3-(4,4,5,5-tetramethyl-1,3,2-dioxaborolan-2-yl)phenyl)amino)-N-methylpyridazine-3-carboxamide (Int. 2) (43 mg, 93 μmol) and XPhos Pd G2 (3.4 mg, 4.0 μmol) were added to a vial with 1,4-dioxane (1 mL) followed by the addition of potassium phosphate tribasic (30 mg, 140 μmol) and water (0.2 mL). The mixture was purged with N₂ and then stirred at 95° C. for 6 hours. After completion, the reaction was quenched with water, extracted with DCM and condensed. The crude was purified by prep-HPLC (0.02% formic acid in water and MeCN) to yield the title compound as the formic salt (16 mg, 50% yield). LCMS calc. for C₃₇H₄₂N₉O₅ [M+H]⁺: m/z=692.3; Found: 692.4.

### Examples 2-3

Examples 2-3 are shown in Table 5 below and were prepared in accordance with the synthetic protocols set forth in Example 1 using the appropriate intermediates, as well as commercial starting materials. The following compounds are TFA salts unless otherwise noted.

TABLE 5
Examples 2-3

| | | | Analytical Data (LCMS |
|---|---|---|---|
| Ex. | Name | Structure | and/or NMR) |
| 2 | 6-(cyclopropanecarboxamido)- 4-((3-(4-((6-(dimethyl-car- bamoyl)pyridin-2-yl)methyl)- 1-isopropyl-2-oxo-2,3,4,5- tetrahydro-1H-benzo[e]- [1,4]diazepin-8-yl)-2- methoxyphenyl)amino)-N- methyl-pyridazine-3- carboxamide | 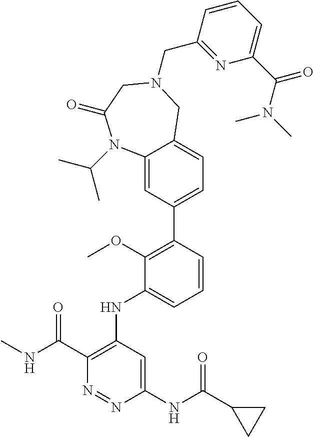 [SMILES: CNC(=O)c1nnc(NC(=O)C2CC2)cc1Nc1cccc(-c2ccc3c(c2)N(C(C)C)C(=O)CN(Cc2cccc(C(=O)N(C)C)n2)C3)c1OC] | LCMS calc. for C₃₈H₄₄N₉O₅ [M + H]⁺: m/z = 706.3; Found 706.8. |
| 3 | 6-(cyclopropanecarbox- amido)-4-((3-(4-(3-(dimethyl- carbamoyl)-2-fluorobenzyl)-1- ethyl-2-oxo-2,3,4,5-tetrahydro- 1H-benzo[e][1,4]diazepin-8- yl)-2-methoxyphenyl)amino)- N-methylpyridazine-3- carboxamide | 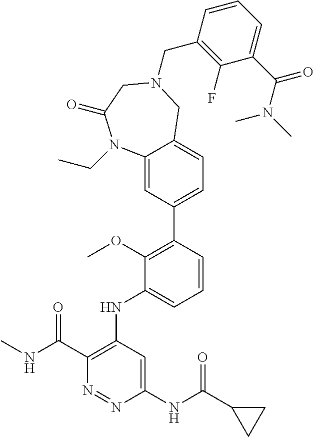 [SMILES: CCN1C(=O)CN(Cc2cccc(C(=O)N(C)C)c2F)Cc2ccc(-c3cccc(Nc4cc(NC(=O)C5CC5)nnc4C(=O)NC)c3OC)cc21] | LCMS calc. for C₃₈H₄₂FN₈O₅ [M + H]⁺: m/z = 709.3 Found 709.4. |

### Example 4. 6-(Cyclopropanecarboxamido)-4-((3-(1-cyclopropyl-4-((6-(dimethylcarbamoyl)-pyridin-2-yl)methyl)-2-oxo-2,3,4,5-tetrahydro-1H-benzo[e][1,4]diazepin-8-yl)-2-methoxy-phenyl)amino)-N-methylpyridazine-3-carboxamide

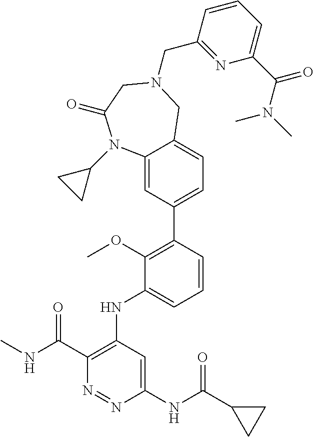 [SMILES: CNC(=O)c1nnc(NC(=O)C2CC2)cc1Nc1cccc(-c2ccc3c(c2)N(C2CC2)C(=O)CN(Cc2cccc(C(=O)N(C)C)n2)C3)c1OC]

### Step 1. 6-((8-Bromo-1-cyclopropyl-2-oxo-1,2,3,5-tetrahydro-4H-benzo[e][1,4]diazepin-4-yl)methyl)-N,N-dimethylpicolinamide

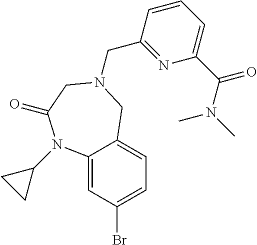 [SMILES: CN(C)C(=O)c1cccc(CN2CC(=O)N(C3CC3)c3cc(Br)ccc3C2)n1]

Cyclopropylboronic acid (43 mg, 0.5 mmol), cupric acetate (45 mg, 0.25 mmol), cesium carbonate (120 mg, 0.37 mmol), 6-((8-bromo-2-oxo-1,2,3,5-tetrahydro-4H-benzo[e][1,4]diazepin-4-yl)methyl)-N,N-dimethylpicolinamide (100 mg, 0.25 mmol) and 4-(dimethylamino)pyridine (91 mg, 0.74 mmol) were added to anhydrous toluene (2 mL). It was stirred at 100° C. After overnight, the reaction was diluted in water and extracted with EtOAc. The organic layer was concentrated and purified by FCC (40-90% Hep:EtOAc) to yield the titled compound (48 mg, 33% yield). LCMS calc. for C₂₁H₂₄BrN₄O₂[M+H]⁺: m/z=443.1/445.1; Found: 445.1.

### Step 2. 6-(Cyclopropanecarboxamido)-4-((3-(1-cyclopropyl-4-((6-(dimethylcarbamoyl)-pyridin-2-yl)methyl)-2-oxo-2,3,4,5-tetrahydro-1H-benzo[e][1,4]diazepin-8-yl)-2-methoxyphenyl) amino)-N-methylpyridazine-3-carboxamide

XPhos Pd G2 (2.0 mg, 2.5 μmol), cesium acetate (15 mg, 0.08 mmol), 6-((8-bromo-1-cyclopropyl-2-oxo-1,2,3,5-tetrahydro-4H-benzo[e][1,4]diazepin-4-yl)methyl)-N,N-dimethylpicolinamide (11 mg, 0.03 mmol), and 6-(cyclopropanecarboxamido)-4-((2-methoxy-3-(4,4,5,5-tetramethyl-1,3,2-dioxaborolan-2-yl)phenyl)amino)-N-methylpyridazine-3-carboxamide (Int. 2) (16 mg, 0.03 mmol) were added in 1,4-dioxane (0.9 mL) and water (0.1 mL) under N₂. After stirring at 100° C. overnight, it was purified by prep-TLC (80% Hep:EtOAc) followed by prep-HPLC (15-35% MeCN in water with 0.05% TFA) to yield the title compound as the TFA salt (2.8 mg, 16% yield). LCMS calc. for C₃₈H₄₂N₉O₅ [M+H]⁺: m/z=704.3; Found: 704.7.

### Example 5. 6-(Cyclopropanecarboxamido)-4-((3-(2-((6-(dimethylcarbamoyl)pyridin-2-yl)methyl)-1-oxo-2,3,4,5-tetrahydro-1H-benzo[c]azepin-7-yl)-2-methoxyphenyl)amino)-N-methylpyridazine-3-carboxamide

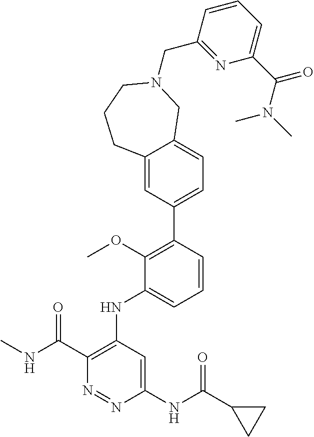 [SMILES: CNC(=O)c1nnc(NC(=O)C2CC2)cc1Nc1cccc(-c2ccc3c(c2)CCCN(Cc2cccc(C(=O)N(C)C)n2)C3)c1OC]

### Step 1. 6-((7-Bromo-1-oxo-1,3,4,5-tetrahydro-2H-benzo[c]azepin-2-yl)methyl)-N,N-dimethylpicolinamide

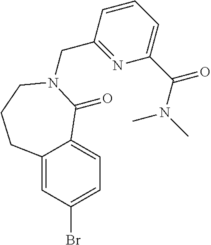 [SMILES: CN(C)C(=O)c1cccc(CN2CCCc3cc(Br)ccc3C2=O)n1]

To a solution of 7-bromo-2,3,4,5-tetrahydro-2-benzazepin-1-one (270 mg, 1.1 mmol) in DMF (5 mL) at 0° C. was added NaH, 60% dispersion mineral oil, (89 mg, 2.2 mmol). After 30 min at 0° C., 6-(chloromethyl)-N,N-dimethylpyridine-2-carboxamide; hydrochloride (390 mg 1.6 mmol) was added and then stirred at rt overnight. The reaction was then slowly quenched with water and extracted with EtOAc followed by DCM. The combined organic layer was then dried over Na₂SO₄, filtered and concentrated. The crude was purified by SiO₂ FCC (10-30% Hex:EtOAc) to yield the title compound as a yellow solid (295 mg, 66% yield). LCMS calc. for C₁₉H₂₁BrN₃O₂ [M+H]⁺: m/z=402.1; Found: 401.9.

### Step 2. 6-(Cyclopropanecarboxamido)-4-((3-(2-((6-(dimethylcarbamoyl)pyridin-2-yl)methyl)-1-oxo-2,3,4,5-tetrahydro-1H-benzo[c]azepin-7-yl)-2-methoxyphenyl)amino)-N-methylpyridazine-3-carboxamide

To a solution of 6-((7-bromo-1-oxo-1,3,4,5-tetrahydro-2H-benzo[c]azepin-2-yl)methyl)-N,N-dimethylpicolinamide (20 mg, 50 μmol), 6-(cyclopropanecarbonylamino)-4-[2-methoxy-3-(4,4,5,5-tetramethyl-1,3,2-dioxaborolan-2-yl)anilino]-N-methylpyridazine-3-carboxamide (Int. 2) (28 mg, 60 μmol), Pd(dppf)Cl₂ (3.6 mg, 5.0 μmol) and K₂CO₃ (14 mg, 99 μmol) in 1,4-dioxane (2 mL) and water (0.4 mL) then stirred at 100° C. After 2 h, the reaction was cooled to rt, filtered and concentrated. The crude was diluted with water and extracted with DCM (10 mL×3). The combined organic phase was washed with brine (sat.) (20 mL×3), dried over Na₂SO₄, filtered, and concentrated. The residue was purified by TLC, followed by prep-HPLC to yield the title compound (10 mg, 29% yield) as the formate salt. LCMS calc. for C₃₆H₃₉N₈O₅ [M+H]⁺: m/z=663.3; Found: 663.4.

### Examples 6-7

Examples 6-7 are shown below in Table 6 and were prepared in accordance with the synthetic protocols set forth in Example 5 using the appropriate intermediates, as well as commercial starting materials. The following compounds are TFA salts unless otherwise noted.

TABLE 6
Examples 6-7

| | | | Analytical Data (LCMS |
|---|---|---|---|
| Ex. | Name | Structure | and/or NMR) |
| 6 | 6-(cyclopropanecarboxamido)- 4-((3-(4-((6-(dimethyl- carbamoyl)pyridin-2- yl)methyl)-5-oxo-2,3,4,5- tetrahydro-benzo[f][1,4]- oxazepin-8-yl)-2- methoxyphenyl)amino)-N- methylpyridazine-3- carboxamide | 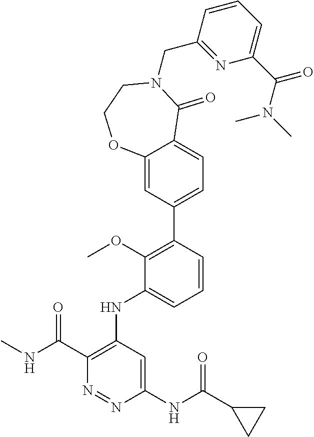 [SMILES: CNC(=O)c1nnc(NC(=O)C2CC2)cc1Nc1cccc(-c2ccc3c(c2)OCCN(Cc2cccc(C(=O)N(C)C)n2)C3=O)c1OC] | LCMS calc. for C₃₅H₃₇N₈O₆ [M + H]⁺: m/z = 665.3; Found 665.2. |
| 7 | 6-(cyclopropanecarboxamido)- 4-((3-(4-((6-(dimethyl- carbamoyl)pyridin-2- yl)methyl)-6-fluoro-5-oxo- 2,3,4,5-tetrahydro- benzo[f][1,4]oxazepin-8-yl)-2- methoxyphenyl)amino)-N- methylpyridazine-3- carboxamide | 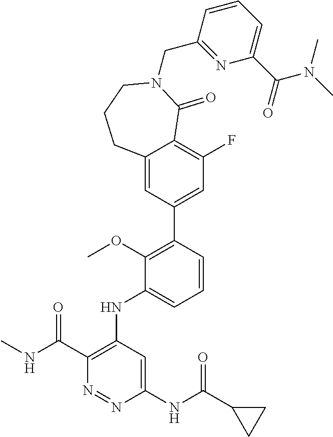 | LCMS calc. for C₃₅H₃₆FN₈O₆ [M + H]⁺: m/z = 683.3; Found: 683.2. ¹H NMR (400 MHz, DMSO- d₆) δ 11.29 (s, 1H), 10.91 (s, 1H), 9.10 (q, J = 4.7 Hz, 1H), 8.08 (s, 1H), 7.89 (t, J = 7.8 Hz, 1H), 7.43 (td, J = 8.7, 2.8 Hz, 3H), 7.30-7.23 (m, 3H), 7.12 (s, 1H), 4.84 (s, 2H), 4.26 (t, J = 5.4 Hz, 2H), 3.65 (t, J = 5.5 Hz, 2H), 3.35 (s, 3H), 2.95 (s, 3H), 2.89 (s, 3H), 2.80 (d, J = 4.7 Hz, 3H), 2.05-2.00 (m, 1H), 0.82-0.75 (m, 4H). |

### Example 8. 6-(Cyclopropanecarboxamido)-4-((3-(4-((6-(dimethylcarbamoyl)pyridin-2-yl)methyl)-1-methyl-5-oxo-2,3,4,5-tetrahydro-1H-benzo[e][1,4]diazepin-8-yl)-2-methoxyphenyl)amino)-N-methylpyridazine-3-carboxamide

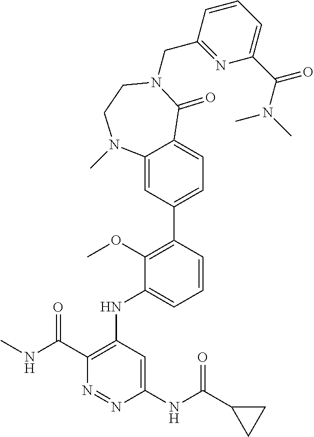 [SMILES: CNC(=O)c1nnc(NC(=O)C2CC2)cc1Nc1cccc(-c2ccc3c(c2)N(C)CCN(Cc2cccc(C(=O)N(C)C)n2)C3=O)c1OC]

### Step 1. 4-Bromo-2-((2-((tert-butoxycarbonyl)amino)ethyl)amino)benzoic acid

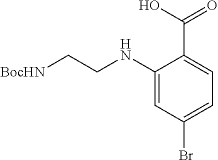 [SMILES: CC(C)(C)OC(=O)NCCNc1cc(Br)ccc1C(=O)O]

A mixture of 4-bromo-2-fluorobenzoic acid (2.0 g, 9.13 mmol) and N-boc-ethylenediamine (4.4 g, 27.0 mmol) in NMP (8 mL) stirred at 120° C. for 16 h. The reaction was then cooled to rt, poured into 1M HCl solution (20 mL), and extracted with EtOAc (40 mL×3). The organic layer was dried over Na₂SO₄, filtered and concentrated under reduced pressure to give the title compound (3.3 g, quant. yield). LCMS calc. for C₉H₁₂BrN₂O₂[M-Boc+H]⁺: m/z=259.0; Found: 259.3.

### Step 2. 2-((2-Aminoethyl)amino)-4-bromobenzoic acid

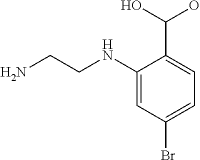 [SMILES: NCCNc1cc(Br)ccc1C(O)O]

To a solution of 4-bromo-2-((2-((tert-butoxycarbonyl)amino)ethyl)amino)benzoic acid (3.3 g, 9.1 mmol) in EtOAc (25 mL) was added 4 M HCl in dioxane (50 mL) dropwise at 0° C. After 2 h at rt, the reaction was condensed to yield the title compound (3.7 g, quant. yield). LCMS calc. for C₉H₁₂BrN₂O₂[M+H]⁺: m/z=259.0; Found: 259.3.

### Step 3. 8-bromo-1,2,3,4-tetrahydro-5H-benzo[e][1,4]diazepin-5-one

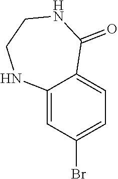 [SMILES: O=C1NCCNc2cc(Br)ccc21]

To a solution of 2-((2-aminoethyl)amino)-4-bromobenzoic acid (500 mg, 1.9 mmol) and HATU (1.1 g, 2.9 mmol) in DCM (5 mL) was added DIPEA (1 mL, 5.8 mmol). After 2 h at rt, the reaction was poured into water and extracted with DCM. The organic layer was washed with brine, dried over Na₂SO₄, filtered and concentrated to dryness. The residue was purified by FCC (2% MeOH/DCM) to yield the title compound as a white solid (300 mg, 65% yield). LCMS calc. for C₉H₁₀BrN₂O [M+H]⁺: m/z=240.9; Found: 240.8.

### Step 4. 8-Bromo-1-methyl-1,2,3,4-tetrahydro-5H-benzo[e][1,4]diazepin-5-one

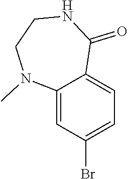

To a mixture of 8-bromo-1,2,3,4-tetrahydro-5H-benzo[e][1,4]diazepin-5-one (75 mg, 0.31 mmol), formaldehyde (47 mg, 0.62 mmol), AcOH (0.1 mL, 0.31 mmol) and NaBH₃CN (39 mg, 0.62 mmol) was added MeOH (1 mL). The reaction was stirred for 1 h, then concentrated under reduced pressure to give a residue. The residue was purified by silica gel column chromatography (5-75% EtOAc/hexane) to yield the title compound (30 mg, 38% yield). LCMS calc. for C₁₀H₁₂BrN₂O [M+H]⁺: m/z=255.0; Found: 255.2.

### Step 5. 6-((8-Bromo-1-methyl-5-oxo-1,2,3,5-tetrahydro-4H-benzo[e][1,4]diazepin-4-yl)methyl)-N,N-dimethylpicolinamide

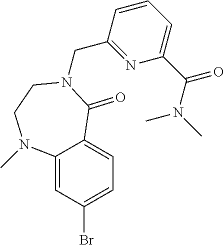 [SMILES: CN(C)C(=O)c1cccc(CN2CCN(C)c3cc(Br)ccc3C2=O)n1]

The title compound was synthesized by a procedure analogous to that outlined in Example 5 Step 1 with appropriate starting materials. LCMS calc. for C₁₉H₂₂BrN₄O₂[M+H]⁺: m/z=417.1; Found: 416.9.

### Step 6. 6-(Cyclopropanecarboxamido)-4-((3-(4-((6-(dimethylcarbamoyl)pyridin-2-yl)methyl)-1-methyl-5-oxo-2,3,4,5-tetrahydro-1H-benzo[e][1,4]diazepin-8-yl)-2-methoxyphenyl) amino)-N-methylpyridazine-3-carboxamide

The title compound, as the formate salt, was synthesized by a procedure analogous to that outlined in Example 5 Step 2 with appropriate starting materials. LCMS calc. for C₃₆H₄₀N₉O₅ [M+H]⁺: m/z=678.3; Found: 678.5. ¹H NMR (400 MHz, DMSO-d₆) δ 11.30 (s, 1H), 10.96 (s, 1H), 9.13 (d, J=4.9 Hz, 1H), 8.16 (s, 1H), 7.91 (d, J=7.8 Hz, 1H), 7.53 (d, J=7.8 Hz, 1H), 7.46-7.44 (m, 1H), 7.44 (s, 1H), 7.42 (d, J=2.0 Hz, 1H), 7.27 (d, J=7.7 Hz, 1H), 7.25-7.21 (m, 2H), 7.13-7.11 (m, 1H), 4.84 (s, 2H), 3.57-3.54 (m, 2H), 3.38 (s, 3H), 3.26-3.23 (m, 2H), 2.98 (s, 3H), 2.92 (s, 3H), 2.83 (d, J=4.8 Hz, 3H), 2.80 (s, 3H), 2.07 (d, J=5.3 Hz, 1H), 0.83-0.79 (m, 4H).

### Examples 9-10

Examples 9-10 are shown below in Table 7 and were prepared in accordance with the synthetic protocols set forth in Example 8 using the appropriate intermediates, as well as commercial starting materials. The following compounds are TFA salts unless otherwise noted.

TABLE 7
Examples 9-10

| | | | Analytical Data (LCMS |
|---|---|---|---|
| Ex. | Name | Structure | and/or NMR) |
| 9 | 6-(cyclopropanecarbox- amido)-4-((3-(4-((6- (dimethyl-carbamoyl)pyridin- 2-yl)methyl)-1-ethyl-5-oxo- 2,3,4,5-tetrahydro-1H- benzo[e][1,4]diazepin-8-yl)- 2-methoxyphenyl)amino)-N- methylpyridazine-3- carboxamide | 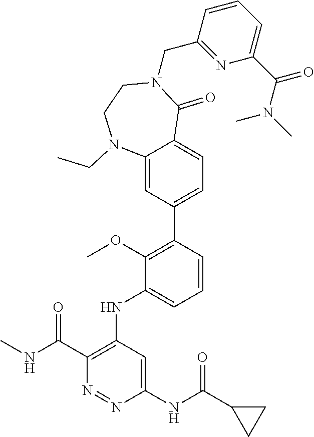 [SMILES: CCN1CCN(Cc2cccc(C(=O)N(C)C)n2)C(=O)c2ccc(-c3cccc(Nc4cc(NC(=O)C5CC5)nnc4C(=O)NC)c3OC)cc21] | LCMS calc. for C₃₇H₄₂N₉O₅ [M + H]⁺: m/z = 692.3; Found 692.3. ¹H NMR (400 MHz, DMSO-d₆) δ 11.29 (s, 1H), 10.95 (s, 1H), 9.12 (d, J = 4.9 Hz, 1H), 8.13 (s, 1H), 7.88 (t, J = 7.8 Hz, 1H), 7.52 (d, J = 7.8 Hz, 1H), 7.46- 7.40 (m, 3H), 7.25 (t, J = 7.8 Hz, 1H), 7.20 (dd, J = 7.7, 1.7 Hz, 1H), 7.15-7.09 (m, 2H), 4.82 (s, 2H), 3.53 (t, J = 5.5 Hz, 2H), 3.35 (s, 3H), 3.25-3.11 (m, 4H), 2.97 (s, 3H), 2.91 (s, 3H), 2.82 (d, J = 4.7 Hz, 3H), 2.05 (h, J = 5.8 Hz, 1H), 1.09 (t, J = 7.0 Hz, 3H), 0.84-0.75 (m, 4H). |
| 10 | 6-(cyclopropanecarbox- amido)-4-((3-(4-((6- (dimethyl-carbamoyl)pyridin- 2-yl)methyl)-1-isopropyl-5- oxo-2,3,4,5-tetrahydro-1H- benzo[e][1,4]diazepin-8-yl)- 2-methoxyphenyl)amino)-N- methylpyridazine-3- carboxamide | 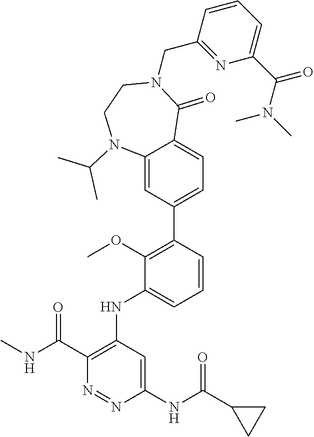 [SMILES: CNC(=O)c1nnc(NC(=O)C2CC2)cc1Nc1cccc(-c2ccc3c(c2)N(C(C)C)CCN(Cc2cccc(C(=O)N(C)C)n2)C3=O)c1OC] | LCMS calc. for C₃₈H₄₄N₉O₅ [M + H]⁺: m/z = 706.3; Found 705.9. ¹H NMR (400 MHz, DMSO-d₆) δ 11.27 (s, 1H), 10.92 (s, 1H), 9.11 (q, J = 4.8 Hz, 1H), 8.14 (s, 1H), 7.86 (t, J = 7.8 Hz, 1H), 7.50 (d, J = 7.9 Hz, 1H), 7.42 (dd, J = 7.7, 3.0 Hz, 3H), 7.26-7.15 (m, 3H), 7.06 (dd, J = 8.0, 1.4 Hz, 1H), 4.80 (s, 2H), 3.79 (p, J = 6.5 Hz, 1H), 3.55 (t, J = 5.6 Hz, 2H), 3.35 (s, 3H), 3.22 (t, J = 5.5 Hz, 2H), 2.96 (s, 3H), 2.90 (s, 3H), 2.81 (d, J = 4.8 Hz, 3H), 2.08-2.01 (m, 1H), 1.14 (d, J = 6.4 Hz, 6H), 0.78 (h, J = 3.1 Hz, 4H). |

### Example 11. 6-(Cyclopropanecarboxamido)-4-((3-(4-((6-(dimethylcarbamoyl)pyridin-2-yl)methyl)-1-methyl-2,5-dioxo-2,3,4,5-tetrahydro-1H-benzo[e][1,4]diazepin-8-yl)-2-methoxyphenyl)amino)-N-methylpyridazine-3-carboxamide

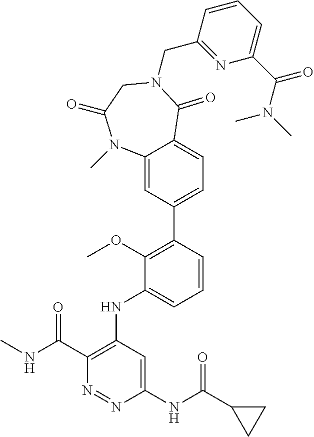 [SMILES: CNC(=O)c1nnc(NC(=O)C2CC2)cc1Nc1cccc(-c2ccc3c(c2)N(C)C(=O)CN(Cc2cccc(C(=O)N(C)C)n2)C3=O)c1OC]

### Step 1. 7-Bromo-1-methyl-2H-benzo[d][1,3]oxazine-2,4(1H)-dione

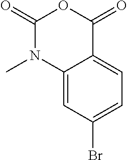

To a solution of 7-bromo-1H-3,1-benzoxazine-2,4-dione (1.0 g, 4.1 mmol) in DMF (10 mL) cooled to 0° C. by ice-bath under nitrogen was added NaH, 60% dispersion mineral oil, (180 mg, 4.5 mmol) in batches. After the addition, the mixture was stirred at 0° C. for 30 mins. Mel (1.2 g, 8.3 mol) was added dropwise and left to stir at rt for 1 h. The reaction was slowly quenched with NH₄Cl (aq) (100 mL) and extracted with EtOAc (3×30 mL). The combined organic layers were washed with brine (2×30 mL), dried over anhydrous Na₂SO₄, filtered and concentrated under reduced pressure to yield the title compound (1.1 g, quant. yield) as a yellow solid. LCMS calc. for C₉H₇BrNO₃ [M+H]⁺: m/z=255.9/257.9; Found 255.5/257.8.

### Step 2. 8-Bromo-1-methyl-3,4-dihydro-1H-benzo[e][1,4]diazepine-2,5-dione

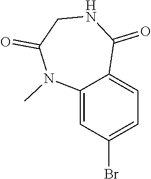

To a mixture of 7-bromo-1-methyl-2H-benzo[d][1,3]oxazine-2,4(1H)-dione (1.8 g, 7.1 mmol) and glycine (0.58 g, 7.7 mmol) in DMF (10 mL) was added Et₃N (2.1 g, 21 mmol). The mixture was stirred at 60° C. for 1 h. The reaction was complete and purified by prep-HPLC on a C₁₈ column (20-70% MeCN in 0.1% TFA(aq), pH=2) to yield the title compound (140 mg, 53% yield). LCMS calc. for C₁₀H₁₀BrN₂O₂[M+H]⁺: m/z=268.9/270.9; Found: 268.9.

### Step 3. 6-((8-Bromo-1-methyl-2,5-dioxo-1, 2,3,5-tetrahydro-4H-benzo[e][1,4]diazepin-4-yl)methyl)-N,N-dimethylpicolinamide

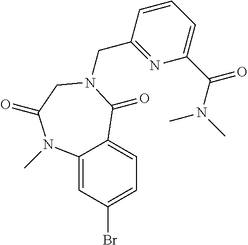 [SMILES: CN(C)C(=O)c1cccc(CN2CC(=O)N(C)c3cc(Br)ccc3C2=O)n1]

The title compound was synthesized by a procedure analogous to that outlined in Example 5 Step 1 with appropriate starting materials. LCMS calc. for C₁₉H₂₀BrN₄O₃[M+H]⁺: m/z=431.1/433.1; Found: 431.1/433.1.

### Step 4. 6-(Cyclopropanecarboxamido)-4-((3-(4-((6-(dimethylcarbamoyl)pyridin-2-yl)methyl)-1-methyl-2,5-dioxo-2,3,4,5-tetrahydro-1H-benzo[e][1,4]diazepin-8-yl)-2-methoxy-phenyl)amino)-N-methylpyridazine-3-carboxamide

The title compound, as the TFA salt, was synthesized by a procedure analogous to that outlined in Example 5 Step 2 with appropriate starting materials. LCMS calc. for C₃₆H₃₈N₉O₆ [M+H]⁺: m/z=692.3; Found: 692.5. ¹H NMR (400 MHz, DMSO-d₆) δ 11.34 (s, 1H), 11.00 (s, 1H), 9.17 (q, J=4.7 Hz, 1H), 8.18 (s, 1H), 7.90 (t, J=7.8 Hz, 1H), 7.83 (d, J=8.1 Hz, 1H), 7.61 (d, J=1.6 Hz, 1H), 7.52 (ddd, J=9.2, 7.4, 2.3 Hz, 2H), 7.45 (d, J=7.6 Hz, 1H), 7.39 (d, J=7.8 Hz, 1H), 7.36-7.27 (m, 2H), 4.92 (s, 2H), 4.28 (d, J=15.2 Hz, 1H), 3.85 (d, J=15.3 Hz, 1H), 3.41 (s, 3H), 3.33 (s, 3H), 2.98 (s, 3H), 2.92-2.82 (m, 6H), 2.09 (dd, J=8.9, 4.0 Hz, 1H), 0.83 (t, J=5.5 Hz, 4H).

### Example 12

Example 12 is shown below in Table 8 and was prepared in accordance with the synthetic protocols set forth in Example 11 using the appropriate intermediates, as well as commercial starting materials. The following compounds are TFA salts unless otherwise noted.

TABLE 8
Example 12

| | | | Analytical Data |
|---|---|---|---|
| Ex. | Name | Structure | (LCMS and/or NMR) |
| 12 | 6-(cyclopropanecarboxamido)- 4-((3-(4-((6-(dimethyl- carbamoyl)pyridin-2- yl)methyl)-1-ethyl-2,5-dioxo- 2,3,4,5-tetrahydro-1H-benzo[e] [1,4]diazepin-8-yl)-2- methoxyphenyl)amino)-N- methylpyridazine-3- carboxamide | 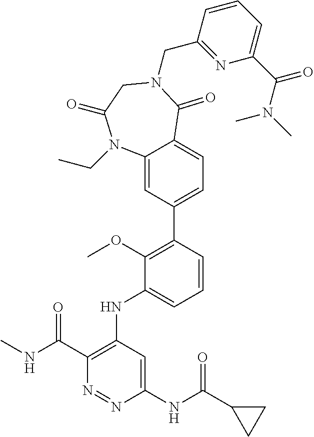 [SMILES: CCN1C(=O)CN(Cc2cccc(C(=O)N(C)C)n2)C(=O)c2ccc(-c3cccc(Nc4cc(NC(=O)C5CC5)nnc4C(=O)NC)c3OC)cc21] | LCMS calc. for C₃₇H₄₀N₉O₆ [M + H]⁺: m/z = 706.3; Found 706.5. |

### Example 13. 6-(Cyclopropanecarboxamido)-4-((3-(4-((6-(dimethylcarbamoyl)pyridin-2-yl)methyl)-1-isopropyl-2,5-dioxo-2,3,4,5-tetrahydro-1H-benzo[e][1,4]diazepin-8-yl)-2-methoxyphenyl)amino)-N-methylpyridazine-3-carboxamide

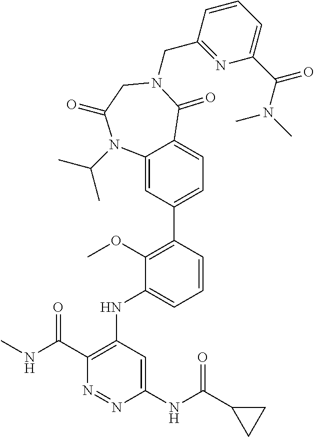 [SMILES: CNC(=O)c1nnc(NC(=O)C2CC2)cc1Nc1cccc(-c2ccc3c(c2)N(C(C)C)C(=O)CN(Cc2cccc(C(=O)N(C)C)n2)C3=O)c1OC]

### Step 1. Methyl ((6-(dimethylcarbamoyl)pyridin-2-yl)methyl)glycinate

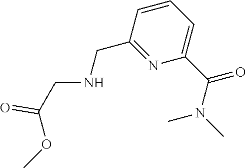 [SMILES: COC(=O)CNCc1cccc(C(=O)N(C)C)n1]

To a mixture of glycine methyl ester; hydrochloride (540 mg, 6.1 mmol) and 6-(chloromethyl)-N,N-dimethylpyridine-2-carboxamide (1.0 g, 5.1 mmol) was added K₂CO₃ (1.1 g, 7.6 mmol) and KI (84 mg, 0.51 mmol) in MeCN (10 mL). The reaction mixture was stirred at 80° C.

After 4 h, the reaction was purified by silica gel column chromatography (50-100% ethyl acetate/hexanes) to yield the title compound (730 mg, 58% yield). LCMS calc. for C₁₂H₁₈N₃O₃ [M+H]⁺: m/z=252.1; Found: 252.0.

### Step 2. Methyl N-(4-bromo-2-nitrobenzoyl)-N-((6-(dimethylcarbamoyl)pyridin-2-yl)methyl)glycinate

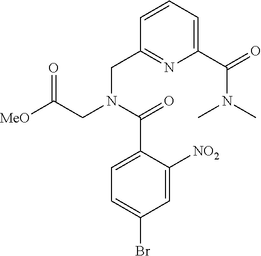 [SMILES: COC(=O)CN(Cc1cccc(C(=O)N(C)C)n1)C(=O)c1ccc(Br)cc1[N+](=O)[O-]]

Methyl ((6-(dimethylcarbamoyl)pyridin-2-yl)methyl)glycinate (270 mg, 1.1 mmol), 4-bromo-2-nitrobenzoic acid (260 mg, 1.1 mmol), HATU (610 mg, 1.6 mmol) and DIPEA (700 mg, 5.4 mmol) were dissolved in DMA (3 mL). The reaction mixture was stirred at rt for 1 h. LCMS showed the reaction was complete. Then the mixture was poured into water and extracted with EtOAc (5 mL). The organic layer was dried over Na₂SO₄, filtered and concentrated under reduced pressure to give a residue. The residue was purified by prep-TLC (hexane:ethyl acetate=0:1) to yield the title compound (107 mg, 21% yield). LCMS calc. for C₁₉H₂₀BrN₄O₆[M+H]⁺: m/z=479.4/481.1; Found 479.1/480.9.

### Step 3. 6-((8-Bromo-2,5-dioxo-1, 2,3,5-tetrahydro-4H-benzo[e][1,4]diazepin-4-yl)methyl)-N,N-dimethylpicolinamide

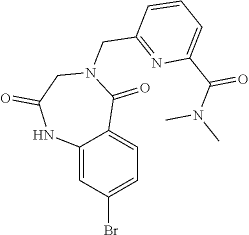 [SMILES: CN(C)C(=O)c1cccc(CN2CC(=O)Nc3cc(Br)ccc3C2=O)n1]

Methyl N-(4-bromo-2-nitrobenzoyl)-N-((6-(dimethylcarbamoyl)pyridin-2-yl)methyl) glycinate (90 mg, 0.19 mmol), Fe (52 mg, 0.94 mmol) and NH₄Cl (100 mg, 1.9 mmol) were dissolved in ethanol (1 mL) and water (0.25 mL). The mixture was stirred at 80° C. for 1 h. Then the mixture was poured into water and extracted with EtOAc (5 mL). The organic layer was dried over Na₂SO₄ and concentrated under reduced pressure to give a residue. The residue was purified by prep-TLC (hexane:ethyl acetate=0:1) to yield the title compound (47 mg, 60% yield). LCMS calc. for C₁₈H₁₈BrN₄O₃[M+H]⁺: m/z=417.1/419.1; Found 417.0/419.0.

### Step 4. 6-((8-Bromo-1-isopropyl-2,5-dioxo-1, 2,3,5-tetrahydro-4H-benzo[e][1,4]diazepin-4-yl)methyl)-N,N-dimethylpicolinamide

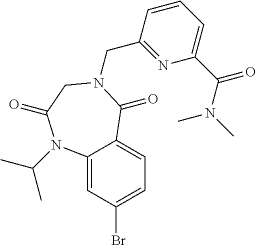 [SMILES: CC(C)N1C(=O)CN(Cc2cccc(C(=O)N(C)C)n2)C(=O)c2ccc(Br)cc21]

6-((8-Bromo-2,5-dioxo-1,2,3,5-tetrahydro-4H-benzo[e][1,4]diazepin-4-yl)methyl)-N,N-dimethylpicolinamide (130 mg, 0.31 mmol), Cs₂CO₃ (310 mg, 0.94 mmol) and 2-iodopropane (160 mg, 0.94 mmol) were dissolved in DMF (1 mL). The reaction mixture was stirred at rt for 16 hrs. Then the mixture was poured into water and extracted with EtOAc (5 mL). The organic layer was dried over Na₂SO₄ and concentrated under reduced pressure to give a residue. The residue was purified by prep-TLC (hexane:ethyl acetate=0:1) to yield the title compound (60 mg, 42% yield). LCMS calc. for C₂₁H₂₄BrN₄O₃[M+H]⁺: m/z=459.1/461.1; Found 459.0/461.0.

### Step 5. 6-(Cyclopropanecarboxamido)-4-((3-(4-((6-(dimethylcarbamoyl)pyridin-2-yl)methyl)-1-isopropyl-2,5-dioxo-2, 3,4,5-tetrahydro-1H-benzo[e][1,4]diazepin-8-yl)-2-methoxyphenyl)amino)-N-methylpyridazine-3-carboxamide

The title compound, as the TFA salt, was synthesized by a procedure analogous to that outlined in Example 5 Step 2 with appropriate starting materials. LCMS calc. for C₃₈H₄₂N₉O₆ [M+H]⁺: m/z=720.3; Found: 720.3. ¹H NMR (400 MHz, DMSO-d₆) δ 11.35 (s, 1H), 10.98 (s, 1H), 9.17 (d, J=4.6 Hz, 1H), 8.16 (s, 1H), 7.89 (s, 1H), 7.79 (s, 1H), 7.56 (s, 2H), 7.52 (dd, J=7.4, 1.6 Hz, 1H), 7.45 (d, J=7.6 Hz, 1H), 7.31 (s, 3H), 4.91 (s, 2H), 4.16 (s, 2H), 3.71 (d, J=15.0 Hz, 1H), 3.41 (s, 3H), 2.98 (s, 3H), 2.93 (s, 3H), 2.86 (d, J=4.6 Hz, 3H), 1.41 (d, J=6.6 Hz, 3H), 1.25 (d, J=6.8 Hz, 3H), 1.23 (s, 1H), 0.82 (t, J=5.8 Hz, 4H).

### Example 14. 6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

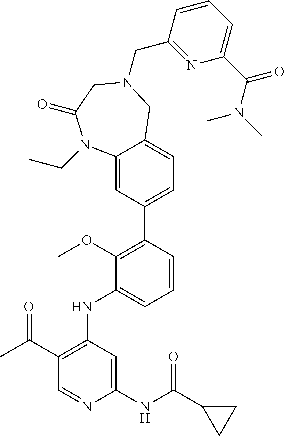 [SMILES: CCN1C(=O)CN(Cc2cccc(C(=O)N(C)C)n2)Cc2ccc(-c3cccc(Nc4cc(NC(=O)C5CC5)ncc4C(C)=O)c3OC)cc21]

### Step 1. 6-((1-Ethyl-2-oxo-8-(4,4,5,5-tetramethyl-1, 3,2-dioxaborolan-2-yl)-1,2,3,5-tetrahydro-4H-benzo[e][1,4]diazepin-4-yl)methyl)-N,N-dimethylpicolinamide

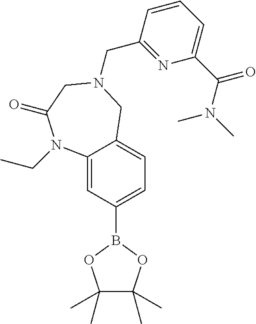 [SMILES: CCN1C(=O)CN(Cc2cccc(C(=O)N(C)C)n2)Cc2ccc(B3OC(C)(C)C(C)(C)O3)cc21]

To a microwave tube was added 6-((8-bromo-1-ethyl-2-oxo-1,2,3,5-tetrahydro-4H-benzo[e][1,4]diazepin-4-yl)methyl)-N,N-dimethylpicolinamide (170 mg, 0.4 mmol) and a magnetic stir bar. The starting material was dissolved in 1,4-dioxane (3 mL) and to this was added bis-(pinacolato)diboron (200 mg, 0.79 mmol) and potassium acetate (120 mg, 1.2 mmol), respectively. The reaction was bubbled with N₂ and to this was added dichloro 1,1′-bisdiphenylphosphino)-ferrocene palladium (II) dichloromethane (32 mg, 0.1 mmol). The vessel was degassed and filled with N₂ (3×). The reaction was heated to 95° C. and allowed to stir overnight. The crude material was filtered through a celite pad and concentrated. The sample was pushed forward without purification (188 mg, quant. yield). LCMS calc. for C₂₆H₃₆BN₄O₄[M+H]⁺: m/z=479.3; Found: 479.5.

### Step 2. 6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

To a solution of N-(5-acetyl-4-((3-bromo-2-methoxyphenyl)amino)pyridin-2-yl)cyclopropanecarboxamide (Int. 3) (61 mg, 0.15 mmol) in dioxane (2 mL) and water (100 μL) was added 6-((1-ethyl-2-oxo-8-(4,4,5,5-tetramethyl-1,3,2-dioxaborolan-2-yl)-1,2,3,5-tetrahydro-4H-benzo[e][1,4]diazepin-4-yl)methyl)-N,N-dimethylpicolinamide (60 mg, 0.13 mmol), cesium acetate (72 mg, 0.38 mmol), Xphos Pd G2 (10 mg, 0.01 mmol). The solution was purged with N₂ for 1 minute. The reaction was stirred at 90° C. for 16 h. Then the reaction mixture was quenched with water, and the water phase was washed with DCM (10 mL×3). The combined organic phase was dried over Na₂SO₄, filtered and concentrated with reduced pressure. The residue was purified by prep-HPLC on a C₁₈ column (5-50% MeCN in 0.1% TFA (aq), pH=2) to afford the title compound (3.0 mg, 3.5% yield) as a white TFA salt. LCMS calc. for C₃₈H₄₂N₇O₅ [M+H]⁺: m/z=676.3; Found: 676.8. ¹H NMR (300 MHz, MeOD) δ 8.81 (s, 1H), 8.10 (t, J=7.8 Hz, 1H), 7.84 (s, 1H), 7.75-7.66 (m, 4H), 7.57 (ddd, J=12.6, 7.8, 1.8 Hz, 2H), 7.45 (t, J=7.8 Hz, 1H), 6.90 (s, 1H), 4.71 (s, 2H), 4.50 (s, 2H), 4.14 (d, J=7.5 Hz, 2H), 3.82 (s, 2H), 3.51 (s, 3H), 3.18 (s, 3H), 3.09 (s, 3H), 2.74 (s, 3H), 1.85 (tt, J=7.7, 4.5 Hz, 1H), 1.30 (t, J=7.1 Hz, 3H), 1.10 (ddt, J=17.1, 7.8, 3.1 Hz, 4H).

### Examples 15-35

Examples 15-35 are shown below in Table 9 and were prepared in accordance with the synthetic protocols set forth in Example 14 using the appropriate intermediates, as well as commercial starting materials. The following compounds are TFA salts unless otherwise noted.

TABLE 9
Examples 15-35

| | | | Analytical Data |
|---|---|---|---|
| Ex. | Name | Structure | (LCMS and/or NMR) |
| 15 | 3-((8-(3-((5-acetyl-2- (cyclopropanecarboxamido)pyri- din-4-yl)amino)-2- methoxyphenyl)-1-ethyl-2- oxo-1,2,3,5-tetrahydro-4H- benzo[e][1,4]diazepin-4- yl)methyl)-2-fluoro-N,N- dimethylbenzamide | 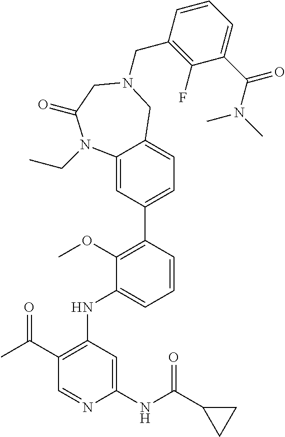 [SMILES: CCN1C(=O)CN(Cc2cccc(C(=O)N(C)C)c2F)Cc2ccc(-c3cccc(Nc4cc(NC(=O)C5CC5)ncc4C(C)=O)c3OC)cc21] | LCMS calc. for C₃₉H₄₂FN₆O₅ [M + H]⁺: m/z = 693.3; Found: 693.4. |
| 16 | 6-((8-(3-((5-acetyl-2- (cyclopropanecarboxamido)pyri- din-4-yl)amino)-2- methoxyphenyl)-1-(2- fluoroethyl)-2-oxo-1,2,3,5- tetrahydro-4H- benzo[e][1,4]diazepin-4- yl)methyl)-N,N- dimethylpicolinamide | 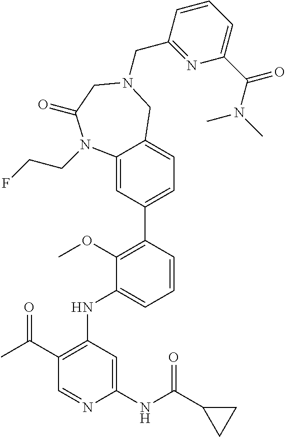 [SMILES: COc1c(Nc2cc(NC(=O)C3CC3)ncc2C(C)=O)cccc1-c1ccc2c(c1)N(CCF)C(=O)CN(Cc1cccc(C(=O)N(C)C)n1)C2] | LCMS calc. for C₃₈H₄₁FN₇O₅ [M + H]⁺: m/z = 694.3; Found: 694.7. ¹H NMR (300 MHz, MeOD) δ 8.80 (s, 1H), 8.06 (t, J = 7.8 Hz, 1H), 7.83 (s, 1H), 7.75- 7.54 (m, 5H), 7.52- 7.38 (m, 2H), 7.08 (s, 1H), 4.79 (d, J = 4.7 Hz, 1H), 4.64 (t, J = 4.7 Hz, 1H), 4.43 (s, 2H), 4.39 (s, 1H), 4.30 (s, 1H), 4.25 (s, 2H), 3.62 (s, 2H), 3.49 (s, 3H), 3.18 (s, 3H), 3.08 (s, 3H), 2.73 (s, 3H), 1.83 (td, J = 7.8, 4.0 Hz, 1H), 1.07 (ddd, J = 17.3, 6.8, 3.1 Hz, 4H). |
| 17 | 6-((8-(3-((5-acetyl-2- (cyclopropanecarboxamido)pyri- din-4-yl)amino)-2- methoxyphenyl)-1-(2,2- difluoroethyl)-2-oxo-1,2,3,5- tetrahydro-4H- benzo[e][1,4]diazepin-4- yl)methyl)-N,N- dimethylpicolinamide | 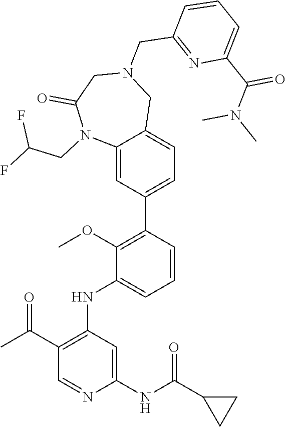 [SMILES: COc1c(Nc2cc(NC(=O)C3CC3)ncc2C(C)=O)cccc1-c1ccc2c(c1)N(CC(F)F)C(=O)CN(Cc1cccc(C(=O)N(C)C)n1)C2] | LCMS calc. for C₃₈H₄₀F₂N₇O₅ [M + H]⁺: m/z = 712.3; Found: 712.7. |
| 18 | 6-((8-(3-((5-acetyl-2- (cyclopropanecarboxamido)pyri- din-4-yl)amino)-2- methoxyphenyl)-2-oxo-1- (2,2,2-trifluoroethyl)-1,2,3,5- tetrahydro-4H- benzo[e][1,4]diazepin-4- yl)methyl)-N,N- dimethylpicolinamide | 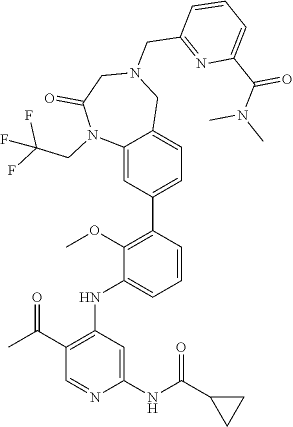 [SMILES: COc1c(Nc2cc(NC(=O)C3CC3)ncc2C(C)=O)cccc1-c1ccc2c(c1)N(CC(F)(F)F)C(=O)CN(Cc1cccc(C(=O)N(C)C)n1)C2] | LCMS calc. for C₃₈H₃₉F₃N₇O₅ [M + H]⁺: m/z = 730.3; Found: 730.8. |
| 19 | 6-((8-(3-((5-acetyl-2- (cyclopropanecarboxamido)pyri- din-4-yl)amino)-2- methoxyphenyl)-1- (cyanomethyl)-2-oxo-1,2,3,5- tetrahydro-4H- benzo[e][1,4]diazepin-4- yl)methyl)-N,N- dimethylpicolinamide | 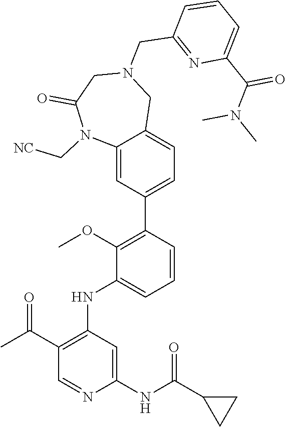 [SMILES: COc1c(Nc2cc(NC(=O)C3CC3)ncc2C(C)=O)cccc1-c1ccc2c(c1)N(CC#N)C(=O)CN(Cc1cccc(C(=O)N(C)C)n1)C2] | LCMS calc. for C₃₈H₃₉N₈O₅ [M + H]⁺: m/z = 687.3; Found: 687.7. ¹H NMR (300 MHz, MeOD) δ 8.77 (s, 1H), 8.04 (t, J = 7.8 Hz, 1H), 7.82 (d, J = 1.5 Hz, 1H), 7.76-7.50 (m, 6H), 7.42 (t, J = 7.8 Hz, 1H), 6.83 (s, 1H), 5.02 (s, 2H), 4.42 (s, 2H), 4.26 (s, 2H), 3.67 (s, 2H), 3.49 (s, 3H), 3.15 (s, 3H), 3.05 (s, 3H), 2.71 (s, 3H), 1.80 (td, J = 7.7, 3.9 Hz, 1H), 1.19- 0.94 (m, 4H). |
| 20 | 6-((8-(3-((5-acetyl-2- (cyclopropanecarboxamido)pyri- din-4-yl)amino)-2- methoxyphenyl)-1-(oxetan-3- yl)-2-oxo-1,2,3,5-tetrahydro- 4H-benzo[e][1,4]diazepin-4- yl)methyl)-N,N- dimethylpicolinamide | 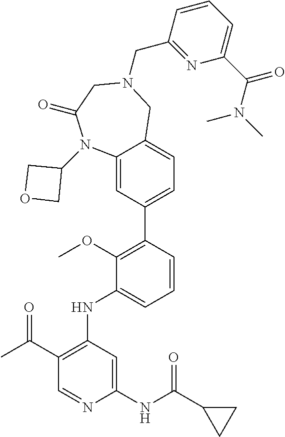 [SMILES: COc1c(Nc2cc(NC(=O)C3CC3)ncc2C(C)=O)cccc1-c1ccc2c(c1)N(C1COC1)C(=O)CN(Cc1cccc(C(=O)N(C)C)n1)C2] | LCMS calc. for C₃₉H₄₂N₇O₆ [M + H]⁺: m/z = 704.3; Found: 704.7. |
| 21 | 6-((8-(3-((5-acetyl-2- (cyclopropanecarboxamido)pyri- din-4-yl)amino)-2- methoxyphenyl)-2-oxo-1- (tetrahydrofuran-3-yl)-1,2,3,5- tetrahydro-4H- benzo[e][1,4]diazepin-4- yl)methyl)-N,N- dimethylpicolinamide | 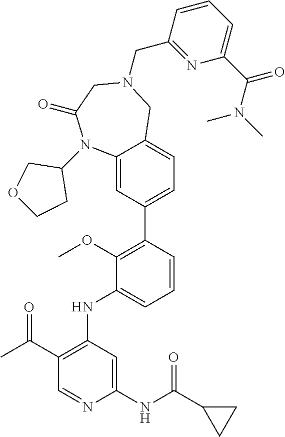 [SMILES: COc1c(Nc2cc(NC(=O)C3CC3)ncc2C(C)=O)cccc1-c1ccc2c(c1)N(C1CCOC1)C(=O)CN(Cc1cccc(C(=O)N(C)C)n1)C2] | LCMS calc. for C₄₀H₄₄N₇O₆ [M + H]⁺: m/z = 718.3; Found: 718.7. |
| 22 | 6-((8-(3-((5-Acetyl-2-((1S,2R)- 2-fluorocyclopropane-1- carboxamido)pyridin-4- yl)amino)-2-methoxyphenyl)- 1-ethyl-2-oxo-1,2,3,5- tetrahydro-4H- benzo[e][1,4]diazepin-4- yl)methyl)-N,N- dimethylpicolinamide | 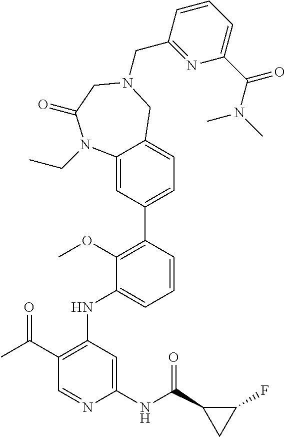 [SMILES: CCN1C(=O)CN(Cc2cccc(C(=O)N(C)C)n2)Cc2ccc(-c3cccc(Nc4cc(NC(=O)[C@@H]5C[C@H]5F)ncc4C(C)=O)c3OC)cc21] | LCMS calc. for C₃₈H₄₁FN₇O₅ [M + H]⁺: m/z = 694.3; Found: 694.7. ¹H NMR (400 MHz, DMSO-d₆) δ 11.12 (s, 1H), 11.02 (s, 1H), 8.83 (s, 1H), 8.01 (s, 1H), 7.91 (s, 1H), 7.68 (s, 1H), 7.63 (d, J = 7.8 Hz, 1H), 7.55 (d, J = 7.1 Hz, 3H), 7.48-7.46 (m, 1H), 7.29-7.26 (m, 2H), 4.83 (dt, J = 64.7, 4.6 Hz, 1H), 4.45 (s, 2H), 4.22 (s, 2H), 3.96 (d, J = 7.2 Hz, 2H), 3.78 (s, 1H), 3.34 (s, 3H), 2.99 (s, 3H), 2.91 (s, 3H), 2.62 (s, 3H), 2.54 (d, J = 7.6 Hz, 1H), 1.52- 1.47 (m, 1H), 1.18 (d, J = 6.6 Hz, 1H), 1.10 (t, J = 7.1 Hz, 3H). |
| 23 | (S)-6-((8-(3-((5-acetyl-2- (spiro[2.2]pentane-1- carboxamido)pyridin-4- yl)amino)-2-methoxyphenyl)- 1-ethyl-2-oxo-1,2,3,5- tetrahydro-4H- benzo[e][1,4]diazepin-4- yl)methyl)-N,N- dimethylpicolinamide | 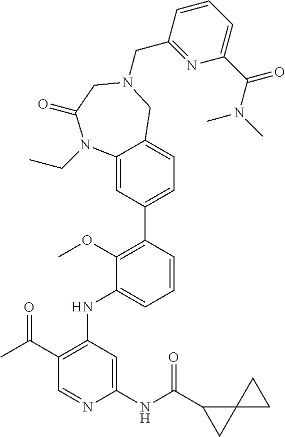 [SMILES: CCN1C(=O)CN(Cc2cccc(C(=O)N(C)C)n2)Cc2ccc(-c3cccc(Nc4cc(NC(=O)C5CC56CC6)ncc4C(C)=O)c3OC)cc21] | LCMS calc. for C₄₀H₄₃N₇O₅ [M + H]⁺: m/z = 702.3; Found 702.7. |
| 24 | (R)-6-((8-(3-((5-acetyl-2- (spiro[2.2]pentane-1- carboxamido)pyridin-4- yl)amino)-2-methoxyphenyl)- 1-ethyl-2-oxo-1,2,3,5- tetrahydro-4H- benzo[e][1,4]diazepin-4- yl)methyl)-N,N- dimethylpicolinamide | 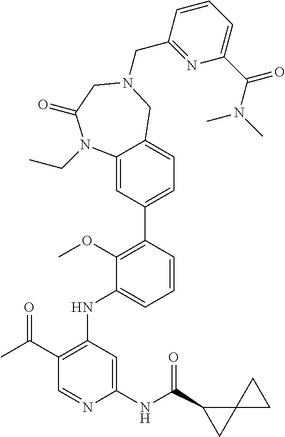 [SMILES: CCN1C(=O)CN(Cc2cccc(C(=O)N(C)C)n2)Cc2ccc(-c3cccc(Nc4cc(NC(=O)[C@@H]5CC56CC6)ncc4C(C)=O)c3OC)cc21] | LCMS calc. for C₄₀H₄₃N₇O₅ [M + H]⁺: m/z = 702.3; Found 702.7. ¹H NMR (400 MHz, DMSO-d₆) δ 11.04 (s, 1H), 10.85 (s, 1H), 8.79 (s, 1H), 8.00 (s, 1H), 7.88 (s, 1H), 7.68 (s, 1H), 7.62 (d, J = 7.8 Hz, 1H), 7.53 (d, J = 5.4 Hz, 3H), 7.49 (d, J = 7.5 Hz, 1H), 7.32-7.28 (m, 2H), 4.41 (s, 1H), 4.19 (s, 2H), 3.96 (d, J = 7.3 Hz, 2H), 3.69 (s, 2H), 3.34 (s, 4H), 2.99 (s, 3H), 2.90 (s, 3H), 2.61 (s, 3H), 2.32-2.29 (m, 1H), 1.33 (d, J = 3.8 Hz, 2H), 1.10 (t, J = 7.1 Hz, 3H), 0.83 (d, J = 8.5 Hz, 2H), 0.69 (d, J = 3.8 Hz, 1H). |
| 25 | 6-((8-(3-((5-acetyl-2-((1R,2S)- 2-fluorocyclopropane-1- carboxamido)pyridin-4- yl)amino)-2-methoxyphenyl)- 1-ethyl-2-oxo-1,2,3,5- tetrahydro-4H- benzo[e][1,4]diazepin-4- yl)methyl)-N,N- dimethylpicolinamide | 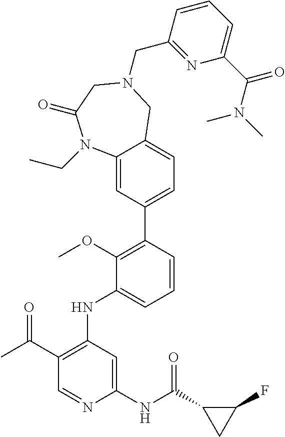 [SMILES: CCN1C(=O)CN(Cc2cccc(C(=O)N(C)C)n2)Cc2ccc(-c3cccc(Nc4cc(NC(=O)[C@H]5C[C@@H]5F)ncc4C(C)=O)c3OC)cc21] | LCMS calc. for C₃₈H₄₁FN₇O₅ [M + H]⁺: m/z = 694.3; Found: 694.7. ¹H NMR (400 MHz, DMSO-d₆) δ 11.15 (s, 1H), 11.03 (s, 1H), 8.83 (s, 1H), 8.01 (t, J = 7.8 Hz, 1H), 7.88 (s, 1H), 7.69 (s, 1H), 7.63 (d, J = 7.8 Hz, 1H), 7.56 (d, J = 6.8 Hz, 3H), 7.49-7.46 (m, 1H), 7.28 (d, J = 4.4 Hz, 2H), 4.83 (dt, J = 64.8, 4.5 Hz, 1H), 4.50 (s, 2H), 4.27 (s, 2H), 3.96 (d, J = 7.3 Hz, 2H), 3.79-3.75 (m, 2H), 3.34 (s, 3H), 2.99 (s, 3H), 2.91 (s, 3H), 2.61 (d, J = 12.2 Hz, 3H), 2.55-2.50 (m, 1H), 1.53-1.48 (m, 1H), 1.21-1.16 (m, 1H), 1.10 (t, J = 7.1 Hz, 3H). |
| 26 | 6-((8-(3-((5-acetyl-2-((1R,2R)- 2-fluorocyclopropane-1- carboxamido)pyridin-4- yl)amino)-2-methoxyphenyl)- 1-ethyl-2-oxo-1,2,3,5- tetrahydro-4H- benzo[e][1,4]diazepin-4- yl)methyl)-N,N- dimethylpicolinamide | 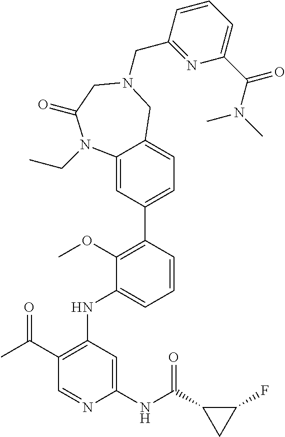 [SMILES: CCN1C(=O)CN(Cc2cccc(C(=O)N(C)C)n2)Cc2ccc(-c3cccc(Nc4cc(NC(=O)[C@H]5C[C@H]5F)ncc4C(C)=O)c3OC)cc21] | LCMS calc. for C₃₈H₄₁FN₇O₅ [M + H]⁺: m/z = 694.3; Found: 694.7. |
| 27 | 6-((8-(3-((5-acetyl-2-((1S,2S)- 2-fluorocyclopropane-1- carboxamido)pyridin-4- yl)amino)-2-methoxyphenyl)- 1-ethyl-2-oxo-1,2,3,5- tetrahydro-4H- benzo[e][1,4]diazepin-4- yl)methyl)-N,N- dimethylpicolinamide | 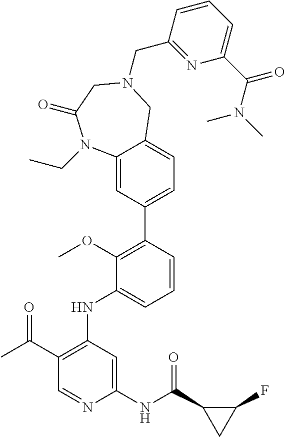 [SMILES: CCN1C(=O)CN(Cc2cccc(C(=O)N(C)C)n2)Cc2ccc(-c3cccc(Nc4cc(NC(=O)[C@@H]5C[C@@H]5F)ncc4C(C)=O)c3OC)cc21] | LCMS calc. for C₃₈H₄₁FN₇O₅ [M + H]⁺: m/z = 694.3; Found: 694.7. ¹H NMR (400 MHz, DMSO-d₆) δ 11.04 (d, J = 11.7 Hz, 2H), 8.81 (s, 1H), 8.00 (t, J = 7.8 Hz, 1H), 7.89 (s, 1H), 7.69 (s, 1H), 7.62 (d, J = 7.8 Hz, 1H), 7.58-7.46 (m, 4H), 7.30 (d, J = 7.4 Hz, 2H), 4.89 (dq, J = 66.2, 5.3 Hz, 1H), 4.46 (s, 2H), 4.23 (s, 2H), 3.96 (d, J = 7.3 Hz, 2H), 3.78 (s, 1H), 3.34 (s, 5H), 2.99 (s, 3H), 2.90 (s, 3H), 2.62 (s, 3H), 2.17 (p, J = 6.9 Hz, 1H), 1.63-1.49 (m, 1H), 1.21-1.05 (m, 5H). |
| 28 | 6-((8-(3-((5-acetyl-2- (cyclopropanecarboxamido)pyri- din-4-yl)amino)-2- methoxyphenyl)-1-cyclobutyl- 2-oxo-1,2,3,5-tetrahydro-4H- benzo[e][1,4]diazepin-4- yl)methyl)-N,N- dimethylpicolinamide | 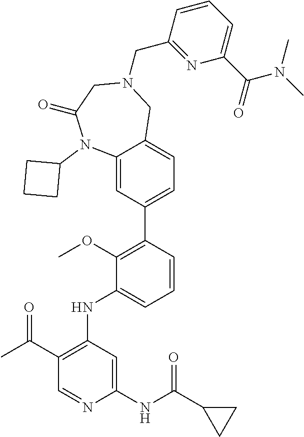 [SMILES: COc1c(Nc2cc(NC(=O)C3CC3)ncc2C(C)=O)cccc1-c1ccc2c(c1)N(C1CCC1)C(=O)CN(Cc1cccc(C(=O)N(C)C)n1)C2] | LC-MS calc. for C₄₀H₄₄N₇O₅ [M + H]⁺: m/z = 702.3; Found: 702.5 |
| 29 | 6-((8-(3-((5-acetyl-2- (cyclopropanecarboxamido)pyri- din-4-yl)amino)-2- methoxyphenyl)-1-(1- fluoropropan-2-yl)-2-oxo- 1,2,3,5-tetrahydro-4H- benzo[e][1,4]diazepin-4- yl)methyl)-N,N- dimethylpicolinamide | 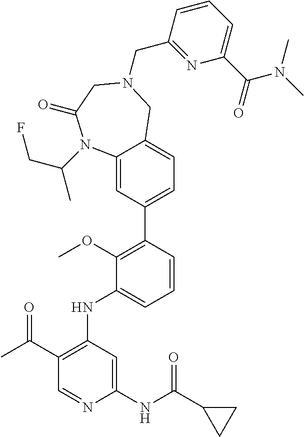 [SMILES: COc1c(Nc2cc(NC(=O)C3CC3)ncc2C(C)=O)cccc1-c1ccc2c(c1)N(C(C)CF)C(=O)CN(Cc1cccc(C(=O)N(C)C)n1)C2] | LC-MS calc. for C₃₉H₄₃FN₇O₅ [M + H]⁺: m/z = 708.3; Found: 708.7 |
| 30 | 6-((8-(3-((5-acetyl-2- (cyclopropanecarboxamido)pyri- din-4-yl)amino)-2- methoxyphenyl)-2-oxo-1- (thiazol-5-ylmethyl)-1,2,3,5- tetrahydro-4H- benzo[e][1,4]diazepin-4- yl)methyl)-N,N- dimethylpicolinamide | 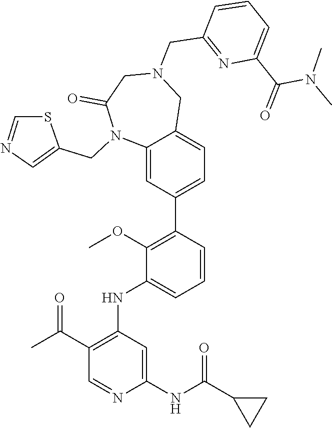 [SMILES: COc1c(Nc2cc(NC(=O)C3CC3)ncc2C(C)=O)cccc1-c1ccc2c(c1)N(Cc1cncs1)C(=O)CN(Cc1cccc(C(=O)N(C)C)n1)C2] | LC-MS calc. for C₄₀H₄₁N₈O₅S [M + H]⁺: m/z = 745.3; Found: 745.6. ¹H NMR (400 MHz, DMSO-d₆) δ 11.07 (d, J = 7.5 Hz, 2H), 8.97 (s, 1H), 8.86 (s, 1H), 7.98 (d, J = 10.0 Hz, 2H), 7.81 (d, J = 14.3 Hz, 2H), 7.62 (d, J = 7.8 Hz, 1H), 7.52 (dd, J = 13.4, 8.8 Hz, 4H), 7.31 (dd, J = 13.2, 7.7 Hz, 2H), 5.42 (s, 2H), 4.26 (s, 2H), 3.88 (s, 2H), 3.47 (s, 2H), 3.34 (s, 3H), 3.02 (s, 3H), 2.94 (s, 3H), 2.66 (s, 3H), 2.01 (d, J = 7.9 Hz, 1H), 0.82 (t, J = 6.6 Hz, 4H). |
| 31 | 6-((8-(3-((5-acetyl-2- (cyclopropanecarboxamido)pyri- din-4-yl)amino)-2- methoxyphenyl)-1- (cyclopropylmethyl)-2-oxo- 1,2,3,5-tetrahydro-4H- benzo[e][1,4]diazepin-4- yl)methyl)-N,N- dimethylpicolinamide | 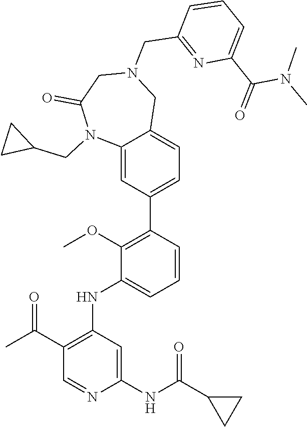 [SMILES: COc1c(Nc2cc(NC(=O)C3CC3)ncc2C(C)=O)cccc1-c1ccc2c(c1)N(CC1CC1)C(=O)CN(Cc1cccc(C(=O)N(C)C)n1)C2] | LC-MS calc. for C₄₀H₄₄N₇O₅ [M + H]⁺: m/z = 702.3; Found: 702.5 |
| 32 | 6-((8-(3-((5-acetyl-2- (cyclopropanecarboxamido)pyri- din-4-yl)amino)-2- methoxyphenyl)-1- cyclopentyl-2-oxo-1,2,3,5- tetrahydro-4H- benzo[e][1,4]diazepin-4- yl)methyl)-N,N- dimethylpicolinamide | 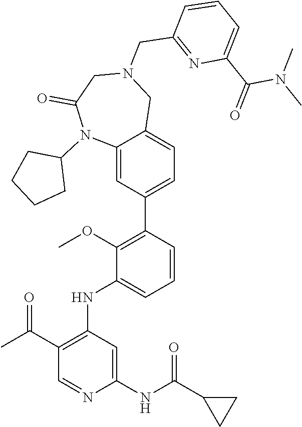 [SMILES: COc1c(Nc2cc(NC(=O)C3CC3)ncc2C(C)=O)cccc1-c1ccc2c(c1)N(C1CCCC1)C(=O)CN(Cc1cccc(C(=O)N(C)C)n1)C2] | LC-MS calc. for C₄₁H₄₆N₇O₅ [M + H]⁺: m/z = 716.4; Found: 716.7 |
| 33 | 6-((8-(3-((5-acetyl-2-((1R,2S)- 2-fluorocyclopropane-1- carboxamido)pyridin-4- yl)amino)-2-methoxyphenyl)- 2-oxo-1-((S)-tetrahydrofuran-3- yl)-1,2,3,5-tetrahydro-4H- benzo[e][1,4]diazepin-4- yl)methyl)-N,N- dimethylpicolinamide | 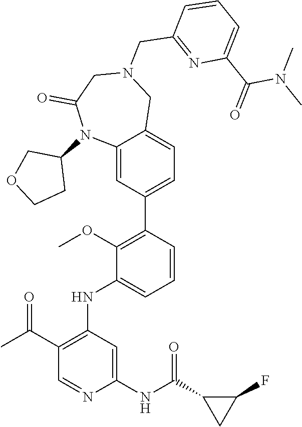 [SMILES: COc1c(Nc2cc(NC(=O)[C@H]3C[C@@H]3F)ncc2C(C)=O)cccc1-c1ccc2c(c1)N([C@H]1CCOC1)C(=O)CN(Cc1cccc(C(=O)N(C)C)n1)C2] | LC-MS calc. for C₄₀H₄₃FN₇O₆ [M + H]⁺: m/z = 736.3; Found: 736.6. ¹H NMR (400 MHz, DMSO-d₆) δ 11.13 (s, 1H), 11.04 (s, 1H), 8.84 (s, 1H), 8.03-7.91 (m, 2H), 7.62 (d, J = 7.8 Hz, 1H), 7.54 (dt, J = 7.8, 2.1 Hz, 4H), 7.48 (dd, J = 7.8, 1.7 Hz, 1H), 7.32-7.21 (m, 2H), 4.93-4.87 (m, 1H), 4.36 (s, 4H), 4.19 (s, 2H), 3.93 (s, 2H), 3.64 (d, J = 8.2 Hz, 1H), 3.42 (s, 2H), 3.33 (s, 3H), 2.99 (s, 3H), 2.91 (s, 3H), 2.63 (s, 3H), 2.57-2.50 (m, 1H), 2.36-1.68 (m, 2H), 1.50 (dddd, J = 20.5, 10.0, 6.4, 3.4 Hz, 1H), 1.19 (dq, J = 12.9, 6.4 Hz, 1H). |
| 34 | 6-((8-(3-((5-acetyl-2-((1R,2S)- 2-fluorocyclopropane-1- carboxamido)pyridin-4- yl)amino)-5-fluoro-2- methoxyphenyl)-2-oxo-1-((S)- tetrahydrofuran-3-yl)-1,2,3,5- tetrahydro-4H- benzo[e][1,4]diazepin-4- yl)methyl)-N,N- dimethylpicolinamide | 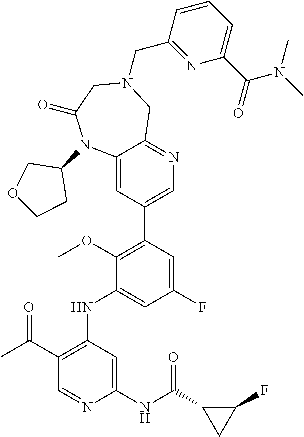 [SMILES: COc1c(Nc2cc(NC(=O)[C@H]3C[C@@H]3F)ncc2C(C)=O)cc(F)cc1-c1cnc2c(c1)N([C@H]1CCOC1)C(=O)CN(Cc1cccc(C(=O)N(C)C)n1)C2] | LC-MS calc. for C₄₀H₄₂F₂N₇O₆ [M + H]⁺: m/z = 754.3; Found: 754.2. |
| 35 | 6-((8-(3-((5-acetyl-2-((1S,2R)- 2-fluorocyclopropane-1- carboxamido)pyridin-4- yl)amino)-5-fluoro-2- methoxyphenyl)-1-(2- fluoroethyl)-2-oxo-1,2,3,5- tetrahydro-4H- benzo[e][1,4]diazepin-4- yl)methyl)-N,N- dimethylpicolinamide | 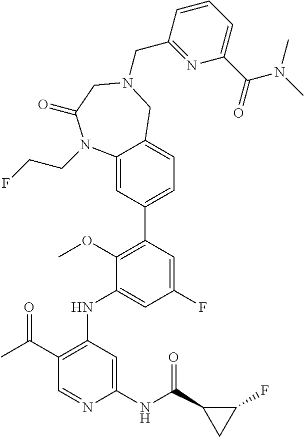 [SMILES: COc1c(Nc2cc(NC(=O)[C@@H]3C[C@H]3F)ncc2C(C)=O)cc(F)cc1-c1ccc2c(c1)N(CCF)C(=O)CN(Cc1cccc(C(=O)N(C)C)n1)C2] | LC-MS calc. for C₃₈H₃₉F₃N₇O₅ [M + H]⁺: m/z = 730.3; Found: 730.2. ¹H NMR (400 MHz, CDCl₃) δ 13.13 (s, 1H), 11.66 (s, 1H), 8.69 (s, 1H), 8.20 (s, 1H), 7.93 (t, J = 7.8 Hz, 1H), 7.69-7.52 (m, 5H), 7.13 (ddd, J = 53.3, 8.4, 3.0 Hz, 2H), 4.92-4.01 (m, 9H), 3.94 (s, 2H), 3.35 (s, 3H), 3.15 (s, 3H), 3.04 (s, 3H), 2.69 (s, 3H), 2.51 (ddt, J = 16.7, 10.1, 6.1 Hz, 1H), 1.63 (dtd, J = 17.5, 6.9, 3.3 Hz, 1H), 1.40 (dq, J = 12.9, 6.5 Hz, 1H). |

### Example 36. 6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

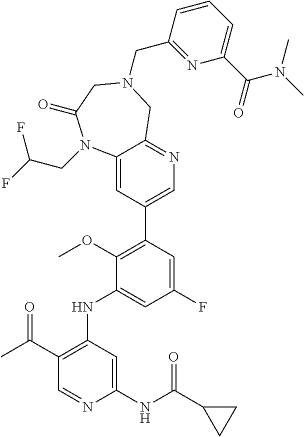 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)C)n1)C2]

Step 1. 6-((8-Bromo-1-(2, 2-difluoroethyl)-2-oxo-1,2,3,5-tetrahydro-4H-pyrido[3,2-e][1,4]diazepin-4-yl)methyl)-N,N-dimethylpicolinamide

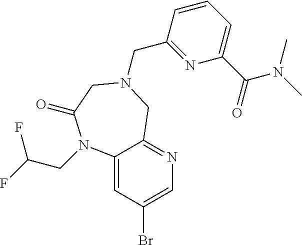 [SMILES: CN(C)C(=O)c1cccc(CN2CC(=O)N(CC(F)F)c3cc(Br)cnc3C2)n1]

To a stirring solution of 6-((8-bromo-2-oxo-1,2,3,5-tetrahydro-4H-pyrido[3,2-e][1,4]diazepin-4-yl)methyl)-N,N-dimethylpicolinamide (Int. 28) (170 mg, 0.42 mmol) in DMF (2 mL) was added cesium carbonate (411 mg, 1.26 mmol) and 1,1-difluoro-2-iodoethane (111 μL, 1.26 mmol) sequentially. The reaction mixture was stirred for 5 hours. The product mixture was diluted with sat. brine (aq) (30 mL) and extracted with a 3:1 CHCl₃/iPrOH mixture (3×30 mL). The combined organic layers were dried over MgSO₄, filtered and concentrated under reduced pressure. The residue obtained was purified by silica gel column chromatography (0-10% MeOH/DCM) to yield the title compound (183 mg, 93% yield). LC-MS calc. for C₁₉H₂₁BrF₂N₅O₂ [M+H]⁺: m/z=468.1/470.1; Found: 468.1/470.1.

### Step 2. 6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

A 20 mL scintillation vial was charged with bis(pinacolato)diboron (73 mg, 0.29 mmol), potassium pivalate (81 mg, 0.58 mmol), [1,1′-bis(diphenylphosphino)ferrocene]dichloro-palladium(II) complex with dichloromethane (16 mg, 0.019 mmol), and 6-((8-bromo-1-(2,2-difluoroethyl)-2-oxo-1,2,3,5-tetrahydro-4H-pyrido[3,2-e][1,4]diazepin-4-yl)methyl)-N,N-dimethylpicolinamide (90 mg, 0.19 mmol). The mixture was dissolved in 1,4-dioxane (1 mL), was sparged with nitrogen gas for 5 minutes and sealed. Then the reaction mixture was heated to 95° C. for 50 minutes. The mixture was cooled to rt, and N-(5-acetyl-4-((3-bromo-5-fluoro-2-methoxyphenyl)-amino)pyridin-2-yl)cyclopropanecarboxamide (Int. 4) (81 mg, 0.19 mmol), potassium carbonate (42 mg, 0.3 mmol), [1,1′-bis(diphenylphosphino)ferrocene]dichloro-palladium(II) complex with dichloromethane (8.2 mg, 0.01 mmol), 1,4-dioxane (1 mL) and water (500 μL) were added to the reaction mixture sequentially. The mixture was sparged with nitrogen gas for 5 minutes and sealed. The reaction mixture was heated to 80° C. for 3 hours. The product mixture was diluted with MeCN (7 mL), filtered, and purified directly by prep-HPLC on a C₁₈ column (18.5-38.5% MeCN in 0.2% TFA(aq)) to afford the TFA salt of the title compound (80 mg, 43% yield). LC-MS calc. for C₃₇H₃₈F₃N₈O₅[M+H]⁺: m/z=731.3; Found: 731.3. ¹H NMR (400 MHz, DMSO-d₆) δ 11.20 (s, 1H), 11.17 (s, 1H), 8.89 (s, 1H), 8.74 (d, J=1.8 Hz, 1H), 8.24 (d, J=1.9 Hz, 1H), 8.02 (s, 1H), 8.00 (t, J=7.7 Hz, 1H), 7.62 (d, J=7.9 Hz, 1H), 7.52 (d, J=7.8 Hz, 1H), 7.48 (dd, J=9.7, 3.0 Hz, 1H), 7.31 (dd, J=9.0, 3.0 Hz, 1H), 6.32 (tt, J=54.8, 4.3 Hz, 1H), 4.51-4.38 (m, 2H), 4.23 (s, 2H), 4.12 (s, 2H), 3.48 (s, 2H), 3.38 (s, 3H), 3.02 (s, 3H), 2.95 (s, 3H), 2.67 (s, 3H), 2.03 (p, J=6.6 Hz, 1H), 0.91-0.79 (m, 4H).

### Examples 37 to 63

Examples 37-63 are shown below in Table 10 and were prepared in accordance with the synthetic protocols set forth in Example 36 using appropriate intermediates, as well as commercial starting materials. The following compounds are TFA salts unless otherwise noted.

TABLE 10
Example 37-63

| | | | Analytical Data (LCMS |
|---|---|---|---|
| Ex. | Name | Structure | and/or NMR) |
| 37 | 6-((8-(3-((5-acetyl-2- ((1S,2R)-2- fluorocyclopropane-1- carboxamido)pyridin- 4-yl)amino)-2- methoxyphenyl)-1- (2,2-difluoroethyl)-2- oxo-1,2,3,5- tetrahydro-4H- pyrido[3,2- e][1,4]diazepin-4- yl)methyl)-N,N- dimethylpicolinamide | 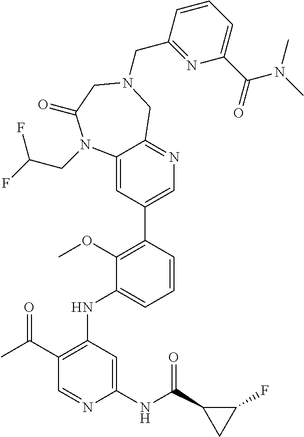 [SMILES: COc1c(Nc2cc(NC(=O)[C@@H]3C[C@H]3F)ncc2C(C)=O)cccc1-c1cnc2c(c1)N(CC(F)F)C(=O)CN(Cc1cccc(C(=O)N(C)C)n1)C2] | LC-MS calc. for C₃₇H₃₈F₃N₈05 [M + H]⁺: m/z = 731.3; Found: 731.3. |
| 38 | 6-((8-(3-((5-acetyl-2- ((1R,2S)-2- fluorocyclopropane-1- carboxamido)pyridin- 4-yl)amino)-2- methoxyphenyl)-1- (2,2-difluoroethyl)-2- oxo-1,2,3,5- tetrahydro-4H- pyrido[3,2- e][1,4]diazepin-4- yl)methyl)-N,N- dimethylpicolinamide | 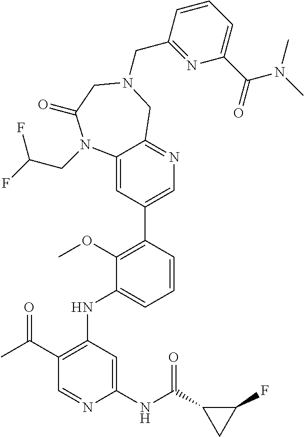 [SMILES: COc1c(Nc2cc(NC(=O)[C@H]3C[C@@H]3F)ncc2C(C)=O)cccc1-c1cnc2c(c1)N(CC(F)F)C(=O)CN(Cc1cccc(C(=O)N(C)C)n1)C2] | LC-MS calc. for C₃₇H₃₈F₃N₈O₅ [M + H]⁺: m/z = 731.3; Found: 731.2. |
| 39 | 6-((8-(3-((5-acetyl-2- ((1R,2R)-2- methylcyclopropane- 1- carboxamido)pyridin- 4-yl)amino)-2- methoxyphenyl)-1- (2,2-difluoroethyl)-2- oxo-1,2,3,5- tetrahydro-4H- pyrido[3,2- e][1,4]diazepin-4- yl)methyl)-N,N- dimethylpicolinamide | 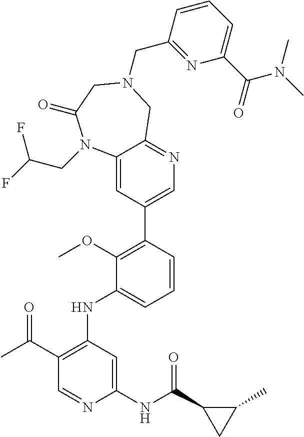 [SMILES: COc1c(Nc2cc(NC(=O)[C@@H]3C[C@H]3C)ncc2C(C)=O)cccc1-c1cnc2c(c1)N(CC(F)F)C(=O)CN(Cc1cccc(C(=O)N(C)C)n1)C2] | LC-MS calc. for C₃₈H₄₁F₂N₈O₅ [M + H]⁺: m/z = 727.3 ; Found: 727.3 |
| 40 | 6-((8-(3-((5-acetyl-2- ((1S,2S)-2- methylcyclopropane- 1- carboxamido)pyridin- 4-yl)amino)-2- methoxyphenyl)-1- (2,2-difluoroethyl)-2- oxo-1,2,3,5- tetrahydro-4H- pyrido[3,2- e][1,4]diazepin-4- yl)methyl)-N,N- dimethylpicolinamide | 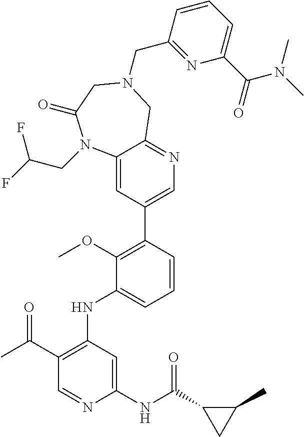 [SMILES: COc1c(Nc2cc(NC(=O)[C@H]3C[C@@H]3C)ncc2C(C)=O)cccc1-c1cnc2c(c1)N(CC(F)F)C(=O)CN(Cc1cccc(C(=O)N(C)C)n1)C2] | LC-MS calc. for C₃₈H₄₁F₂N₈0₅ [M + H]⁺: m/z = 727.3 ; Found: 727.3 |
| 41 | (S)-6-((8-(3-((5-acetyl- 2- (cyclopropanecarboxa- mido)pyridin-4- yl)amino)-5-fluoro-2- methoxyphenyl)-2- oxo-1- (tetrahydrofuran-3-yl)- 1,2,3,5-tetrahydro-4H- pyrido[3,2- e][1,4]diazepin-4- yl)methyl)-N,N- dimethylpicolinamide | 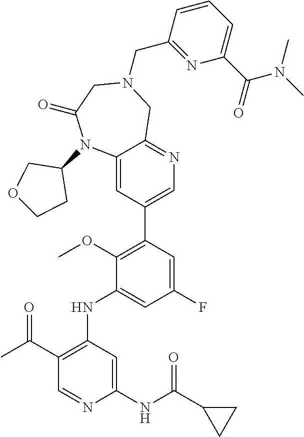 [SMILES: COc1c(Nc2cc(NC(=O)C3CC3)ncc2C(C)=O)cc(F)cc1-c1cnc2c(c1)N([C@H]1CCOC1)C(=O)CN(Cc1cccc(C(=O)N(C)C)n1)C2] | LC-MS calc. for C₃₉H₄₂FN₈0₆ [M + H]⁺: m/z = 737.3 ; Found: 737.3. ¹H NMR (400 MHz, DMSO-d₆) δ 11.17 (s, 1H), 11.09 (s, 1H), 8.89 (s, 1H), 8.73 (d, J = 1.9 Hz, 1H), 8.09 (s, 1H), 8.04-7.89 (m, 2H), 7.65- 7.58 (m, 1H), 7.52 (d, J = 7.7 Hz, 1H), 7.47 (dd, J = 9.8, 3.0 Hz, 1H), 7.23 (dd, J = 9.0, 3.0 Hz, 1H), 4.96 (s, 1H), 4.21 (s, 2H), 4.11 (s, 2H), 4.01-3.92 (m, 2H), 3.88 (s, 1H), 3.68 (q, J = 7.8 Hz, 1H), 3.39 (s, 3H), 3.35 (s, 2H), 3.02 (s, 3H), 2.95 (s, 3H), 2.67 (s, 3H), 2.36- 2.20 (m, 1H), 2.04 (dt, J = 12.5, 6.3 Hz, 2H), 1.01- 0.74 (m, 4H). |
| 42 | 6-((8-(3-((5-acetyl-2- ((1R,2S)-2- fluorocyclopropane-1- carboxamido)pyridin- 4-yl)amino)-5-fluoro- 2-methoxyphenyl)-2- oxo-1-((S)- tetrahydrofuran-3-yl)- 1,2,3,5-tetrahydro-4H- pyrido[3,2- e][1,4]diazepin-4- yl)methyl)-N,N- dimethylpicolinamide | 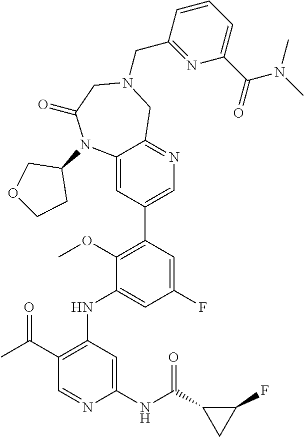 [SMILES: COc1c(Nc2cc(NC(=O)[C@H]3C[C@@H]3F)ncc2C(C)=O)cc(F)cc1-c1cnc2c(c1)N([C@H]1CCOC1)C(=O)CN(Cc1cccc(C(=O)N(C)C)n1)C2] | LC-MS calc. for C₃₉H₄₁F₂N₈O₆ [M + H]⁺: m/z = 755.3; Found: 755.3 |
| 43 | 6-((8-(3-((5-acetyl-2- (cyclopropanecarboxa- mido)pyridin-4- yl)amino)-5-fluoro-2- methoxyphenyl)-1-(2- fluoroethyl)-2-oxo- 1,2,3,5-tetrahydro-4H- pyrido[3,2- e][1,4]diazepin-4- yl)methyl)-N,N- dimethylpicolinamide | 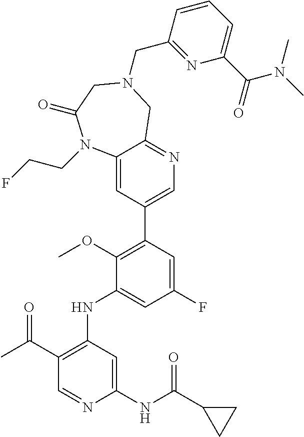 [SMILES: COc1c(Nc2cc(NC(=O)C3CC3)ncc2C(C)=O)cc(F)cc1-c1cnc2c(c1)N(CCF)C(=O)CN(Cc1cccc(C(=O)N(C)C)n1)C2] | LC-MS calc. for C₃₇H₃₉F₂N₈O₅ [M + H]⁺: m/z = 713.3; Found: 713.3. ¹H NMR (400 MHz, DMSO- d₆) δ 11.19 (s, 1H), 11.18 (s, 1H), 8.88 (s, 1H), 8.74 (d, J = 1.8 Hz, 1H), 8.19 (d, J = 1.9 Hz, 1H), 8.05-7.95 (m, 2H), 7.63 (d, J = 7.9 Hz, 1H), 7.54 (d, J = 7.7 Hz, 1H), 7.48 (dd, J = 9.7, 3.0 Hz, 1H), 7.30 (dd, J = 9.0, 3.0 Hz, 1H), 4.65 (dt, J = 47.3, 4.8 Hz, 2H), 4.39- 4.19 (m, 6H), 3.57 (s, 2H), 3.39 (s, 2H), 3.02 (s, 2H), 2.95 (s, 2H), 2.66 (s, 3H), 2.02 (ddd, J = 12.5, 7.2, 5.3 Hz, 1H), 0.89-0.80 (m, 4H). |
| 44 | 6-((8-(3-((5-acetyl-2- ((1R,2S)-2- fluorocyclopropane-1- carboxamido)pyridin- 4-yl)amino)-5-fluoro- 2-methoxyphenyl)-1- (2-fluoroethyl)-2-oxo- 1,2,3,5-tetrahydro-4H- pyrido[3,2- e][1,4]diazepin-4- yl)methyl)-N,N- dimethylpicolinamide | 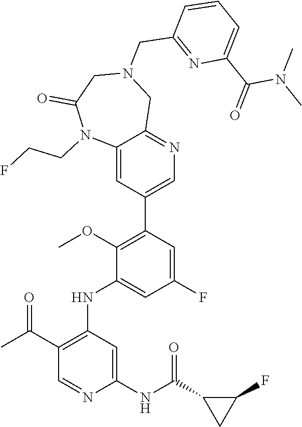 [SMILES: COc1c(Nc2cc(NC(=O)[C@H]3C[C@@H]3F)ncc2C(C)=O)cc(F)cc1-c1cnc2c(c1)N(CCF)C(=O)CN(Cc1cccc(C(=O)N(C)C)n1)C2] | LC-MS calc. for C₃₇H₃₈F₃N₈O₅ [M + H]⁺: m/z = 731.3; Found: 731.3 |
| 45 | 6-((8-(3-((5-acetyl-2- ((1R,2S)-2- fluorocyclopropane-1- carboxamido)pyridin- 4-yl)amino)-5-fluoro- 2-methoxyphenyl)-1- (2,2-difluoroethyl)-2- oxo-1,2,3,5- tetrahydro-4H- pyrido[3,2- e][1,4]diazepin-4- yl)methyl)-N,N- dimethylpicolinamide | 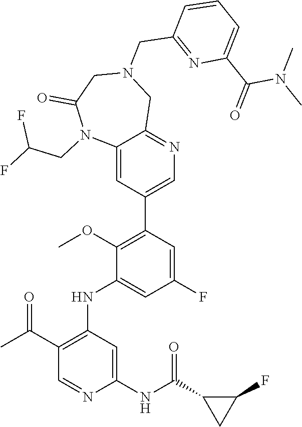 [SMILES: COc1c(Nc2cc(NC(=O)[C@H]3C[C@@H]3F)ncc2C(C)=O)cc(F)cc1-c1cnc2c(c1)N(CC(F)F)C(=O)CN(Cc1cccc(C(=O)N(C)C)n1)C2] | LC-MS calc. for C₃₇H₃₇F₄N₈O₅ [M + H]⁺: m/z = 749.3; Found: 749.3 |
| 46 | 6-((8-(3-((5-acetyl-2- ((1S,2R)-2- fluorocyclopropane-1- carboxamido)pyridin- 4-yl)amino)-5-fluoro- 2-methoxyphenyl)-1- (2,2-difluoroethyl)-2- oxo-1,2,3,5- tetrahydro-4H- pyrido[3,2- e][1,4]diazepin-4- yl)methyl)-N,N- dimethylpicolinamide | 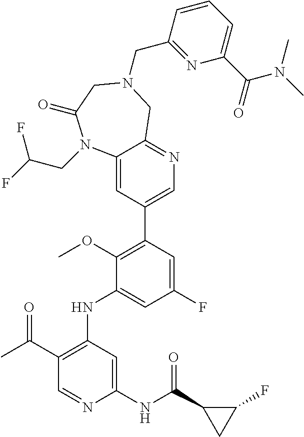 [SMILES: COc1c(Nc2cc(NC(=O)[C@@H]3C[C@H]3F)ncc2C(C)=O)cc(F)cc1-c1cnc2c(c1)N(CC(F)F)C(=O)CN(Cc1cccc(C(=O)N(C)C)n1)C2] | LC-MS calc. for C₃₇H₃₇F₄N₈O₅ [M + H]⁺: m/z = 749.3; Found: 749.5 |
| 47 | 6-((8-(3-((5-acetyl-2- ((1S,2R)-2- fluorocyclopropane-1- carboxamido)pyridin- 4-yl)amino)-5-fluoro- 2-methoxyphenyl)-1- isopropyl-2-oxo- 1,2,3,5-tetrahydro-4H- pyrido[3,2- e][1,4]diazepin-4- yl)methyl)-N,N- dimethylpicolinamide | 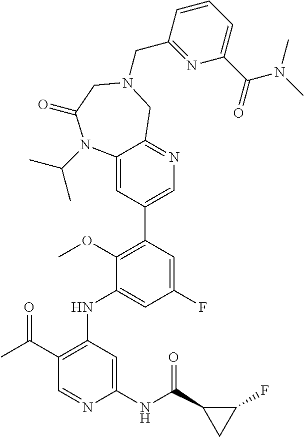 [SMILES: COc1c(Nc2cc(NC(=O)[C@@H]3C[C@H]3F)ncc2C(C)=O)cc(F)cc1-c1cnc2c(c1)N(C(C)C)C(=O)CN(Cc1cccc(C(=O)N(C)C)n1)C2] | LC-MS calc. for C₃₈H₄₁F₂N₈O₅ [M + H]⁺: m/z = 727.3; Found: 727.4 |
| 48 | 6-((8-(3-((5-acetyl-2- (cyclopropanecarboxa- mido)pyridin-4- yl)amino)-2- methoxyphenyl)-1- isobutyl-2-oxo- 1,2,3,5-tetrahydro-4H- pyrido[3,2- e][1,4]diazepin-4- yl)methyl)-N,N- dimethylpicolinamide | 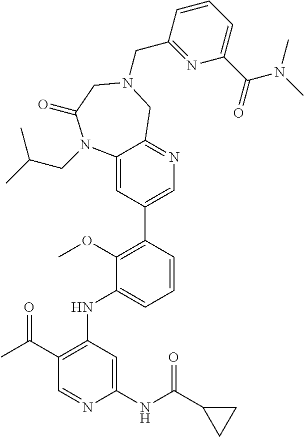 [SMILES: COc1c(Nc2cc(NC(=O)C3CC3)ncc2C(C)=O)cccc1-c1cnc2c(c1)N(CC(C)C)C(=O)CN(Cc1cccc(C(=O)N(C)C)n1)C2] | LC-MS calc. for C₃₉H₄₅N₈O₅ [M + H]⁺: m/z = 705.4; Found: 705.3. ¹H NMR (300 MHz, CDCl₃) δ 11.37 (s, 1H), 10.81 (s, 1H), 8.70-8.60 (m, 2H), 8.30-8.25 (m, 1H), 8.25-8.20 (m, 1H), 7.95-7.90 (m, 1H), 7.85-7.80 (m, 1H), 7.70-7.65 (m, 1H), 7.60-7.50 (m, 2H), 7.40-7.35 (m, 1H), 4.12 (s, 2H), 4.04 (s, 2H), 3.87 (d, J = 6.4 Hz, 2H), 3.45 (s, 3H), 3.34 (s, 2H), 3.16 (s, 3H), 3.13 (s, 3H), 2.70 (s, 3H), 2.00-1.90 (m, 1H), 1.85-1.80 (m, 1H), 1.15-1.05 (m, 2H), 1.00-0.95 (m, 2H), 0.95 (d, J = 6.4 Hz, 4H). |
| 49 | 6-((8-(3-((5-acetyl-2- ((1R,2S)-2- fluorocyclopropane-1- carboxamido)pyridin- 4-yl)amino)-5-fluoro- 2-methoxyphenyl)-1- cyclobutyl-2-oxo- 1,2,3,5-tetrahydro-4H- pyrido[3,2- e][1,4]diazepin-4- yl)methyl)-N,N- dimethylpicolinamide | 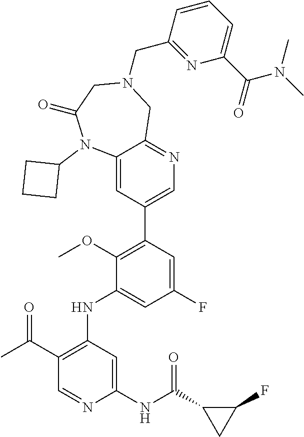 [SMILES: COc1c(Nc2cc(NC(=O)[C@H]3C[C@@H]3F)ncc2C(C)=O)cc(F)cc1-c1cnc2c(c1)N(C1CCC1)C(=O)CN(Cc1cccc(C(=O)N(C)C)n1)C2] | LC-MS calc. for C₃₉H₄₁F₂N₈O₅ [M + H]⁺: m/z = 739.3; Found: 739.3. |
| 50 | 6-((8-(3-((5-acetyl-2- ((1R,2S)-2- fluorocyclopropane-1- carboxamido)pyridin- 4-yl)amino)-5-fluoro- 2-methoxyphenyl)-6- fluoro-2-oxo-1-((S)- tetrahydrofuran-3-yl)- 1,2,3,5-tetrahydro-4H- benzo[e][1,4]diazepin- 4-yl)methyl)-N,N- dimethylpicolinamide | 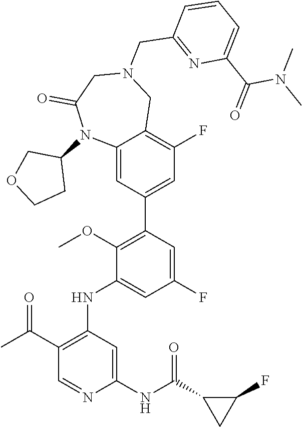 [SMILES: COc1c(Nc2cc(NC(=O)[C@H]3C[C@@H]3F)ncc2C(C)=O)cc(F)cc1-c1cc(F)c2c(c1)N([C@H]1CCOC1)C(=O)CN(Cc1cccc(C(=O)N(C)C)n1)C2] | LC-MS calc. for C₄₀H₄₁F₃N₇O₆ [M + H]⁺: m/z = 772.3; Found: 772.3. |
| 51 | 6-((8-(3-((5-acetyl-2- ((1S,2R)-2- fluorocyclopropane-1- carboxamido)pyridin- 4-yl)amino)-2- methoxyphenyl)-2- oxo-1-((R)- tetrahydrofuran-3-yl)- 1,2,3,5-tetrahydro-4H- benzo[e][1,4]diazepin- 4-yl)methyl)-N,N- dimethylpicolinamide | 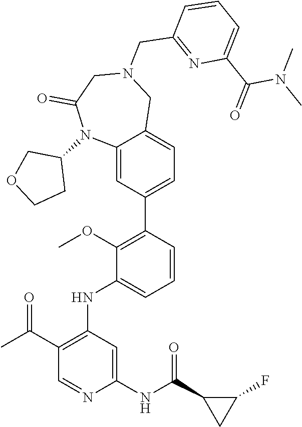 [SMILES: COc1c(Nc2cc(NC(=O)[C@@H]3C[C@H]3F)ncc2C(C)=O)cccc1-c1ccc2c(c1)N([C@@H]1CCOC1)C(=O)CN(Cc1cccc(C(=O)N(C)C)n1)C2] | LC-MS calc. for C₄₀H₄₃FN₇O₆ [M + H]⁺: m/z = 736.3; Found: 736.6. ¹H NMR (400 MHz, DMSO-d₆) δ 11.15 (s, 1H), 11.06 (s, 1H), 8.87 (s, 1H), 8.03 (t, J = 7.7 Hz, 1H), 7.97 (s, 1H), 7.65 (d, J = 7.8 Hz, 1H), 7.57 (d, J = 7.0 Hz, 4H), 7.51 (dd, J = 7.7, 1.7 Hz, 1H), 7.31 (t, J = 7.7 Hz, 1H), 7.26 (d, J = 7.7 Hz, 1H), 4.41 (s, 4H), 3.99-3.93 (m, 2H), 3.67 (d, J = 8.3 Hz, 1H), 3.47 (s, 2H), 3.36 (s, 3H), 3.03 (s, 3H), 2.94 (s, 3H), 2.66 (s, 3H), 2.61-2.52 (m, 1H), 2.25 (s, 2H), 1.59-1.46 (m, 1H), 1.21 (dt, J = 13.0, 6.5 Hz, 1H). |
| 52 | 6-((8-(3-((5-acetyl-2- ((1R,2S)-2- fluorocyclopropane-1- carboxamido)pyridin- 4-yl)amino)-2- methoxyphenyl)-1- (2,2-difluoroethyl)-2- oxo-1,2,3,5- tetrahydro-4H- benzo[e][1,4]diazepin- 4-yl)methyl)-N,N- dimethylpicolinamide | 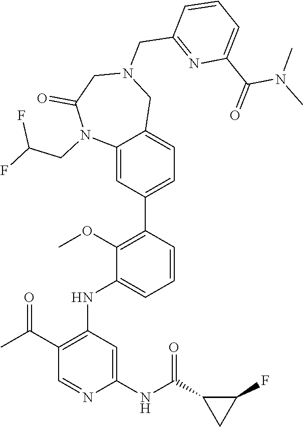 [SMILES: COc1c(Nc2cc(NC(=O)[C@H]3C[C@@H]3F)ncc2C(C)=O)cccc1-c1ccc2c(c1)N(CC(F)F)C(=O)CN(Cc1cccc(C(=O)N(C)C)n1)C2] | LC-MS calc. for C₃₈H₃₉F₃N₇O₅ [M + H]⁺: m/z = 730.3; Found: 730.5. |
| 53 | 6-((8-(3-((5-acetyl-2- ((1R,2S)-2- fluorocyclopropane-1- carboxamido)pyridin- 4-yl)amino)-2- methoxyphenyl)-1-(2- fluoroethyl)-2-oxo- 1,2,3,5-tetrahydro-4H- benzo[e][1,4]diazepin- 4-yl)methyl)-N,N- dimethylpicolinamide | 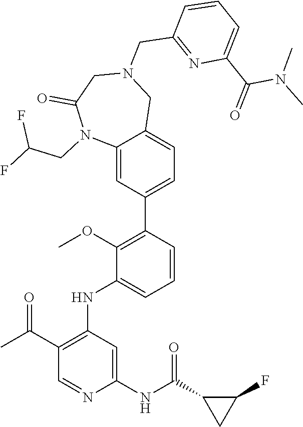 [SMILES: COc1c(Nc2cc(NC(=O)[C@H]3C[C@@H]3F)ncc2C(C)=O)cccc1-c1ccc2c(c1)N(CC(F)F)C(=O)CN(Cc1cccc(C(=O)N(C)C)n1)C2] | LC-MS calc. for C₃₈H₄₀F₂N₇O₅ [M + H]⁺: m/z = 712.3; Found: 712.5. |
| 54 | 6-((8-(3-((5-acetyl-2- (cyclopropanecarboxa- mido)pyridin-4- yl)amino)-6-fluoro-2- methoxyphenyl)-1- ethyl-2-oxo-1,2,3,5- tetrahydro-4H- benzo[e][1,4]diazepin- 4-yl)methyl)-N,N- dimethylpicolinamide | 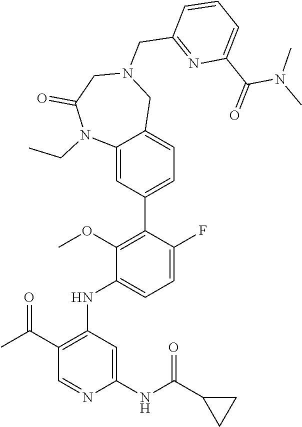 [SMILES: CCN1C(=O)CN(Cc2cccc(C(=O)N(C)C)n2)Cc2ccc(-c3c(F)ccc(Nc4cc(NC(=O)C5CC5)ncc4C(C)=O)c3OC)cc21] | LC-MS calc. for C₃₈H₄₁FN₇O₅ [M + H]⁺: m/z = 694.3; Found: 694.4. |
| 55 | 6-((8-(3-((5-acetyl-2- ((1S,2R)-2- fluorocyclopropane-1- carboxamido)pyridin- 4-yl)amino)-2- methoxyphenyl)-2- oxo-1,2,3,5- tetrahydro-4H- benzo[e][1,4]diazepin- 4-yl)methyl)-N,N- dimethylpicolinamide | 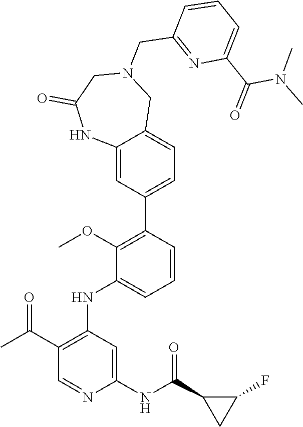 [SMILES: COc1c(Nc2cc(NC(=O)[C@@H]3C[C@H]3F)ncc2C(C)=O)cccc1-c1ccc2c(c1)NC(=O)CN(Cc1cccc(C(=O)N(C)C)n1)C2] | LC-MS calc. for C₃₆H₃₇FN₇O₅ [M + H]⁺: m/z = 666.3; Found: 666.5. |
| 56 | 6-((8-(3-((5-acetyl-2- ((1R,2S)-2- fluorocyclopropane-1- carboxamido)pyridin- 4-yl)amino)-2- methoxy-5- (trifluoromethyl)phenyl)- 1-methyl-2-oxo- 1,2,3,5-tetrahydro-4H- pyrido[3,2- e][1,4]diazepin-4- yl)methyl)-N,N- dimethylpicolinamide | 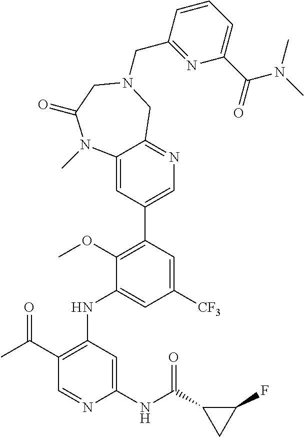 [SMILES: COc1c(Nc2cc(NC(=O)[C@H]3C[C@@H]3F)ncc2C(C)=O)cc(C(F)(F)F)cc1-c1cnc2c(c1)N(C)C(=O)CN(Cc1cccc(C(=O)N(C)C)n1)C2] | LC-MS calc. for C₃₇H₃₇F₄N₈O₅ [M + H]⁺: m/z = 749.3; Found: 749.2. |
| 57 | 6-((8-(3-((5-acetyl-2- ((1R,2S)-2- fluorocyclopropane-1- carboxamido)pyridin- 4-yl)amino)-2- methoxy-5- (trifluoromethyl)phenyl)- 2-oxo-1,2,3,5- tetrahydro-4H- benzo[e][1,4]diazepin- 4-yl)methyl)-N,N- dimethylpicolinamide | 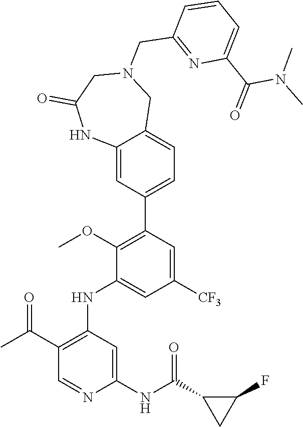 [SMILES: COc1c(Nc2cc(NC(=O)[C@H]3C[C@@H]3F)ncc2C(C)=O)cc(C(F)(F)F)cc1-c1ccc2c(c1)NC(=O)CN(Cc1cccc(C(=O)N(C)C)n1)C2] | LC-MS calc. for C₃₇H₃₆F₄N₇O₅ [M + H]⁺: m/z = 734.3; Found: 734.2. ¹H NMR (400 MHz, DMSO-d₆) δ 11.15 (s, 2H), 10.12 (s, 1H), 8.90 (s, 1H), 8.34 (s, 1H), 7.99 (s, 1H), 7.92 (t, J = 7.7 Hz, 1H), 7.81 (s, 1H), 7.55 (d, J = 7.6 Hz, 1H), 7.48 (s, 1H), 7.44 (d, J = 7.7 Hz, 1H), 7.31 (d, J = 12.9 Hz, 2H), 4.92 (s, 1H), 4.76 (s, 1H), 3.87 (d, J = 15.1 Hz, 4H), 3.44 (s, 3H), 2.99 (s, 3H), 2.91 (s, 3H), 2.66 (s, 3H), 1.59-1.46 (m, 2H), 1.20 (dd, J = 13.4, 6.7 Hz, 2H). |
| 58 | 6-((8-(3-((5-acetyl-2- ((1R,2S)-2- fluorocyclopropane-1- carboxamido)pyridin- 4-yl)amino)-2- methoxyphenyl)-2- oxo-1-((S)- tetrahydrofuran-3-yl)- 1,2,3,5-tetrahydro-4H- pyrido[3,2- e][1,4]diazepin-4- yl)methyl)-N,N- dimethylpicolinamide | 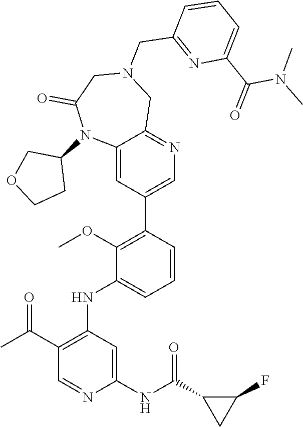 [SMILES: COc1c(Nc2cc(NC(=O)[C@H]3C[C@@H]3F)ncc2C(C)=O)cccc1-c1cnc2c(c1)N([C@H]1CCOC1)C(=O)CN(Cc1cccc(C(=O)N(C)C)n1)C2] | LC-MS calc. for C₃₉H₄₂FN₈O₆ [M + H]⁺: m/z = 737.3; Found: 737.2. |
| 59 | 6-((8-(3-((5-acetyl-2- ((1S,2R)-2- fluorocyclopropane-1- carboxamido)pyridin- 4-yl)amino)-2- methoxyphenyl)-2- oxo-1-((S)- tetrahydrofuran-3-yl)- 1,2,3,5-tetrahydro-4H- pyrido[3,2- e][1,4]diazepin-4- yl)methyl)-N,N- dimethylpicolinamide | 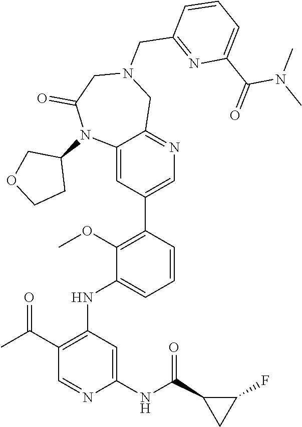 [SMILES: COc1c(Nc2cc(NC(=O)[C@@H]3C[C@H]3F)ncc2C(C)=O)cccc1-c1cnc2c(c1)N([C@H]1CCOC1)C(=O)CN(Cc1cccc(C(=O)N(C)C)n1)C2] | LC-MS calc. for C₃₉H₄₂FN₈O₆ [M + H]⁺: m/z = 737.3; Found: 737.2. |
| 60 | 6-((8-(3-((5-acetyl-2- ((1S,2R)-2- fluorocyclopropane-1- carboxamido)pyridin- 4-yl)amino)-5-fluoro- 2-methoxyphenyl)-2- oxo-1-((S)- tetrahydrofuran-3-yl)- 1,2,3,5-tetrahydro-4H- pyrido[3,2- e][1,4]diazepin-4- yl)methyl)-N,N- dimethylpicolinamide | 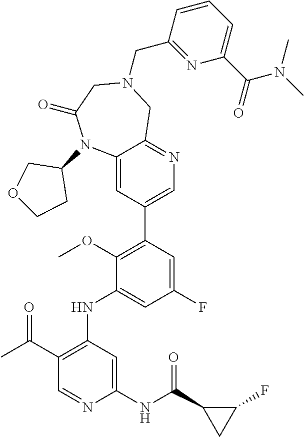 [SMILES: COc1c(Nc2cc(NC(=O)[C@@H]3C[C@H]3F)ncc2C(C)=O)cc(F)cc1-c1cnc2c(c1)N([C@H]1CCOC1)C(=O)CN(Cc1cccc(C(=O)N(C)C)n1)C2] | LC-MS calc. for C₃₉H₄₁F₂N₈O₆ [M + H]⁺: m/z = 755.3; Found: 755.2. |
| 61 | 6-((8-(3-((5-acetyl-2- (cyclopropanecarboxa- mido)pyridin-4- yl)amino)-5-fluoro-2- methoxyphenyl)-1-(2- fluoro-2- methylpropyl)-2-oxo- 1,2,3,5-tetrahydro-4H- pyrido[3,2- e][1,4]diazepin-4- yl)methyl)-N,N- dimethylpicolinamide | 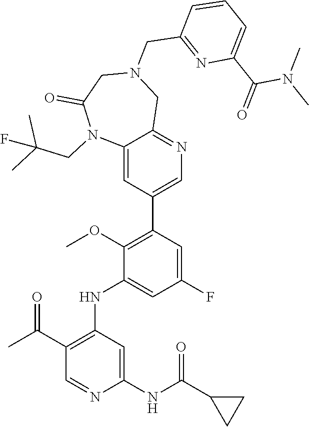 [SMILES: COc1c(Nc2cc(NC(=O)C3CC3)ncc2C(C)=O)cc(F)cc1-c1cnc2c(c1)N(CC(C)(C)F)C(=O)CN(Cc1cccc(C(=O)N(C)C)n1)C2] | LC-MS calc. for C₃₉H₄₃F₂N₈O₅ [M + H]⁺: m/z = 741.3; Found: 741.3. |
| 62 | 6-((8-(3-((5-acetyl-2- (cyclopropanecarboxa- mido)pyridin-4- yl)amino)-5-fluoro-2- methoxy-2- methoxyphenyl)-1-(2- methylpropyl)-2-oxo- 1,2,3,5-tetrahydro-4H- pyrido[3,2- e][1,4]diazepin-4- yl)methyl)-N,N- dimethylpicolinamide | 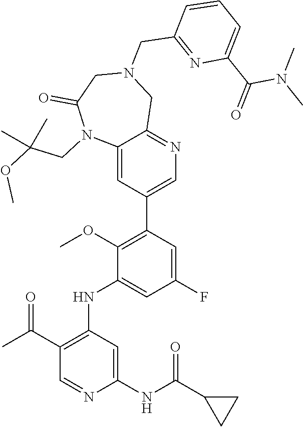 [SMILES: COc1c(Nc2cc(NC(=O)C3CC3)ncc2C(C)=O)cc(F)cc1-c1cnc2c(c1)N(CC(C)(C)OC)C(=O)CN(Cc1cccc(C(=O)N(C)C)n1)C2] | LC-MS calc. for C₄₀H₄₆FN₈O₆ [M + H]⁺: m/z = 753.4; Found: 753.4. |
| 63 | 6-((8-(3-((5-acetyl-2- (cyclopropanecarboxa- mido)pyridin-4- yl)amino)-5-fluoro-2- methoxyphenyl)-1- isobutyl-2-oxo- 1,2,3,5-tetrahydro-4H- pyrido[3,2- e][1,4]diazepin-4- yl)methyl)-N,N- dimethylpicolinamide | 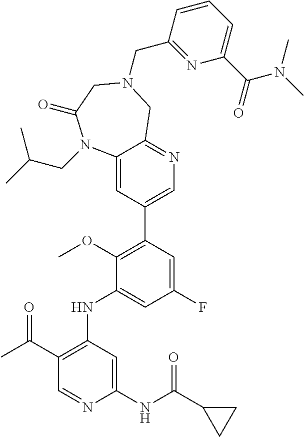 [SMILES: COc1c(Nc2cc(NC(=O)C3CC3)ncc2C(C)=O)cc(F)cc1-c1cnc2c(c1)N(CC(C)C)C(=O)CN(Cc1cccc(C(=O)N(C)C)n1)C2] | LC-MS calc. for C₃₉H₄₄FN₈O₅ [M + H]⁺: m/z = 723.3; Found: 723.3. |

### Example 64. 3-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylbenzamide

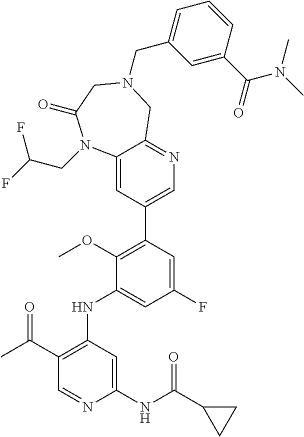 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)C)c1)C2]

### Step 1. tert-butyl 8-bromo-1-(2,2-difluoroethyl)-2-oxo-1,2,3,5-tetrahydro-4H-pyrido[3,2-e][1,4]diazepine-4-carboxylate

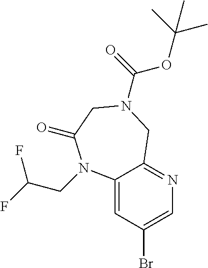 [SMILES: CC(C)(C)OC(=O)N1CC(=O)N(CC(F)F)c2cc(Br)cnc2C1]

The title compound was prepared in accordance with the synthetic protocols set forth in Example 36 Step 1 using the appropriate intermediates. LC-MS calc. for C₁₅H₁₉BrF₂N₃O₃ [M+H]⁺: m/z=406.1/408.1; Found: 406.1/408.1.

### Step 2. tert-butyl 8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepine-4-carboxylate

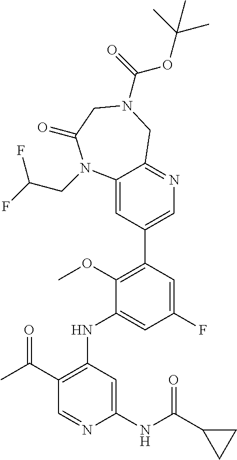 [SMILES: COc1c(Nc2cc(NC(=O)C3CC3)ncc2C(C)=O)cc(F)cc1-c1cnc2c(c1)N(CC(F)F)C(=O)CN(C(=O)OC(C)(C)C)C2]

The title compound was prepared in accordance with the synthetic protocols set forth in Example 36 Step 2 using the appropriate intermediates. LC-MS calc. for C₃₃H₃₆F₃N₆O₆[M+H]⁺: m/z=669.3; Found: 669.2.

### Step 3. N-(5-acetyl-4-((3-(1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide

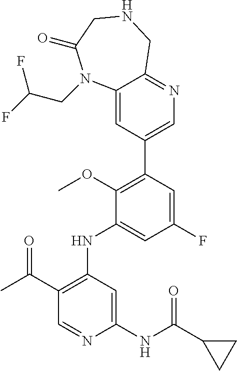 [SMILES: COc1c(Nc2cc(NC(=O)C3CC3)ncc2C(C)=O)cc(F)cc1-c1cnc2c(c1)N(CC(F)F)C(=O)CNC2]

To a stirring solution of tert-butyl 8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepine-4-carboxylate (1820 mg, 2.73 mmol) in 1,4-dioxane (12 mL) was added HCl (12 mL, 48 mmol, 4 M in 1,4-dioxane). The suspension was stirred at rt for 3 hours, then directly condensed to afford the title compound (1650 mg, 99% yield) as the HCl salt. LC-MS calc. for C₂₈H₂₈F₃N₆O₄[M+H]⁺: m/z=569.2; Found: 569.2.

### Step 4. 3-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylbenzamide

To a stirring solution of 3-(hydroxymethyl)-N,N-dimethylbenzamide (6 mg, 0.033 mmol) and triethylamine (21 μL, 0.15 mmol) in DMSO (0.5 mL) at 0° C. was added SO₃ pyridine (16 mg, 0.099 mmol) in DMSO (0.5 mL). The resulting solution was allowed to warm to rt and stirred for 2 h before another portion of SO₃ pyridine (25 mg, 0.16 mmol) in DMSO (100 μL) was added. The reaction was stirred an additional 45 minutes before MeCN (0.5 mL) was added and the reaction was returned to 0° C. Acetic acid (14 μL, 0.25 mmol) and N-(5-acetyl-4-((3-(1-(2,2-difluoroethyl)-2-oxo-2,3,4,5-tetrahydro-1H-pyrido[3,2-e][1,4]diazepin-8-yl)-5-fluoro-2-methoxyphenyl)amino)-pyridin-2-yl)cyclopropanecarboxamide HCl (10 mg, 0.0165 mmol) were added sequentially and the reaction was allowed to stir at rt for 30 minutes before STAB (17.5 mg, 0.083 mmol) was added. After stirring at rt overnight, the reaction mixture was diluted in MeCN and purified by prep-LCMS (CSH-C18, 19.2-39.2% MeCN/water with 0.2% TFA over 5 min) to afford the title compound (2.4 mg, 15% yield) as a TFA salt. LC-MS calc. for C₃₈H₃₉F₃N₇O₅[M+H]⁺: m/z=730.3; Found: 730.3.

### Example 65. 6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methyl-N-(2,2,2-trifluoroethyl)picolinamide

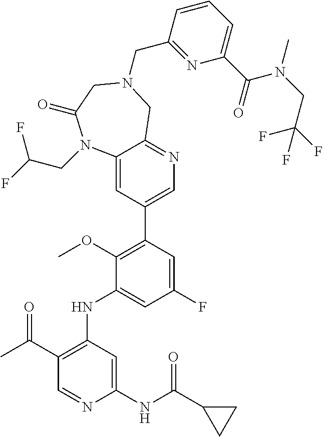 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)CC(F)(F)F)n1)C2]

### Step 1. Methyl 6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)picolinate

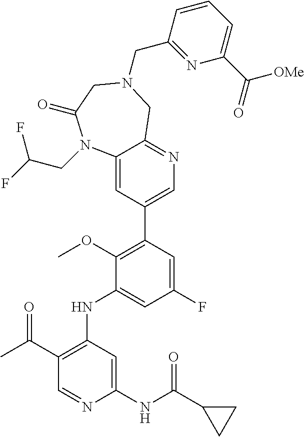 [SMILES: COC(=O)c1cccc(CN2CC(=O)N(CC(F)F)c3cc(-c4cc(F)cc(Nc5cc(NC(=O)C6CC6)ncc5C(C)=O)c4OC)cnc3C2)n1]

To a solution of N-(5-acetyl-4-((3-(1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide; HCl (204 mg, 0.34 mmol) in DMF (3 mL) was added acetic acid (0.19 mL, 3.4 mmol) and methyl 6-formylpicolinate (170 mg, 1.0 mmol). The solution stirred at 35° C. for 20 min then sodium triacetoxyborohydride (360 mg, 1.7 mmol) was added. After 1 hour, the reaction was cooled to rt and quenched with sat. sodium bicarbonate (aq) and extracted with EtOAc (2×). The combined organic layers were dried with sodium sulfate, filtered and condensed. Crude was purified by silica gel column chromatography (0-100% EtOAc/Hexanes then 0-10% MeOH/EtOAc) to elute the product as a yellow-orange solid. LCMS calc. for C₃₆H₃₅F₃N₇O₆[M+H]⁺: m/z=718.3; Found: 718.2.

### Step 2. 6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)picolinic acid

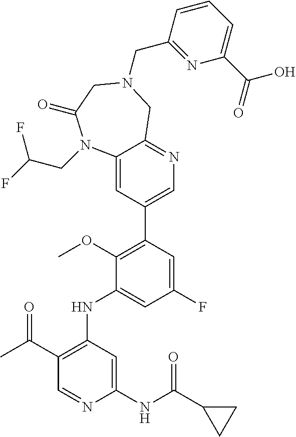 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)O)n1)C2]

To a solution of methyl 6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)picolinate (210 mg, 0.29 mmol) in methanol (10 mL), THF (10 mL) and water (10 mL) was added lithium hydroxide hydrate (1:1:1) (62 mg, 1.5 mmol). The solution was stirred for 30 min. The reaction mixture was quenched with citric acid to adjust the pH to 6~7. The mixture was then extracted with EtOAc (3×). The combined organic layers were washed with brine, dried over sodium sulfate, filtered and condensed to yield the title compound (150 mg, 73% yield). LCMS calc. for C₃₅H₃₃F₃N₇O₆[M+H]⁺: m/z=704.2; Found: 704.1.

### Step 3. 6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methyl-N-(2,2,2-trifluoroethyl)picolinamide

To a solution of 6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)picolinic acid (7.0 mg, 0.01 mmol) in DMF (0.50 mL) was added HATU (4.2 mg, 0.01 mmol) and DIPEA (7.0 μL, 0.04 mmol) followed by 2,2,2-trifluoro-N-methylethanamine; hydrobromide (3.0 mg, 0.01 mmol). After 2 h, the reaction was diluted in acetonitrile and purified by prep-LCMS (CSH-C18, 25.6-45.6% MeCN/water with 0.2% TFA over 5 min). Product fractions were lyophilized to yield the title compound as a TFA salt (3.2 mg, 28% yield). LCMS calc. for C₃₈H₃₇F₆N₈O₅[M+H]⁺: m/z=799.3; Found: 799.2.

### Examples 66-70

Examples 66-70 are shown below in Table 11 and were prepared in accordance with the synthetic protocols set forth in Example 65 using the appropriate intermediates, as well as commercial starting materials. The following compounds are TFA salts unless otherwise noted.

TABLE 11
Examples 66-70

| | | | Analytical Data |
|---|---|---|---|
| Ex. | Name | Structure | (LCMS and/or NMR) |
| 66 | N-(5-acetyl-4-((3-(1-(2,2- difluoroethyl)-4-((6-(3- hydroxypyrrolidine-1- carbonyl)pyridin-2- yl)methyl)-2-oxo-2,3,4,5- tetrahydro-1H-pyrido[3,2- e][1,4]diazepin-8-yl)-5- fluoro-2-methoxyphenyl)- amino)pyridin-2- yl)cyclopropane- carboxamide | 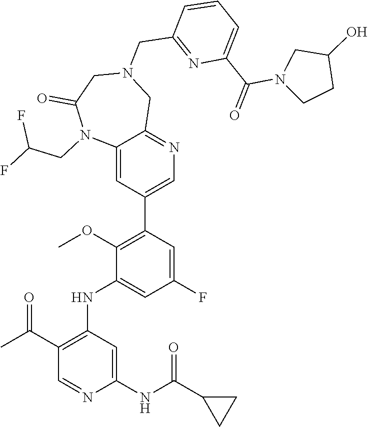 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N3CCC(O)C3)n1)C2] | LCMS calc. for C₃₉H₄₀F₃N₈O₆ [M + H]⁺: m/z = 773.3; Found: 773.4. |
| 67 | N-(5-acetyl-4-((3-(1-(2,2- difluoroethyl)-2-oxo-4-((6- (pyrrolidine-1- carbonyl)pyridin-2- yl)methyl)-2,3,4,5- tetrahydro-1H-pyrido[3,2- e][1,4]diazepin-8-yl)-5- fluoro-2-methoxyphenyl)- amino)pyridin-2- yl)cyclopropane- carboxamide | 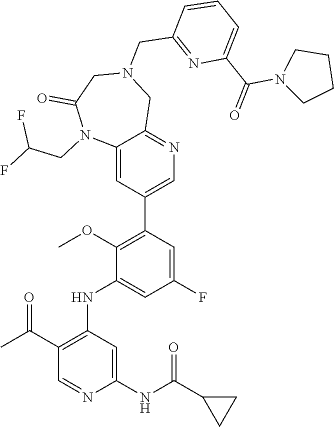 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N3CCCC3)n1)C2] | LCMS calc. for C₃₉H₄₀F₃N₈O₅ [M + H]⁺: m/z = 757.3; Found: 757.2. |
| 68 | 6-((8-(3-((5-acetyl-2- (cyclopropanecarboxamido) pyridin-4-yl)amino)-5- fluoro-2-methoxyphenyl)- 1-(2,2-difluoroethyl)-2- oxo-1,2,3,5-tetrahydro-4H- pyrido[3,2-e][1,4]diazepin- 4-yl)methyl)-N-(2,2- difluoroethyl)-N- methylpicolinamide | 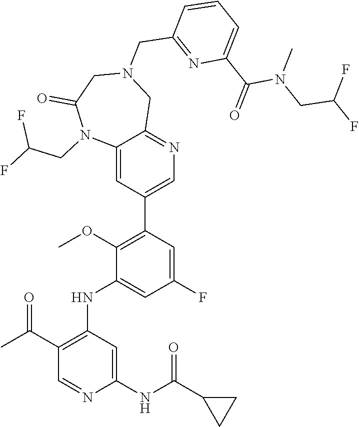 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)CC(F)F)n1)C2] | LCMS calc. for C₃₈H₃₈F₅N₈O₅ [M + H]⁺: m/z = 781.3; Found: 781.2. |
| 69 | 6-((8-(3-((5-acetyl-2- (cyclopropanecarboxamido) pyridin-4-yl)amino)-5- fluoro-2-methoxyphenyl)- 1-(2,2-difluoroethyl)-2- oxo-1,2,3,5-tetrahydro-4/I- pyrido[3,2-e][1,4]diazepin- 4-yl)methyl)-N-(2- hydroxyethyl)-N- methylpicolinamide | 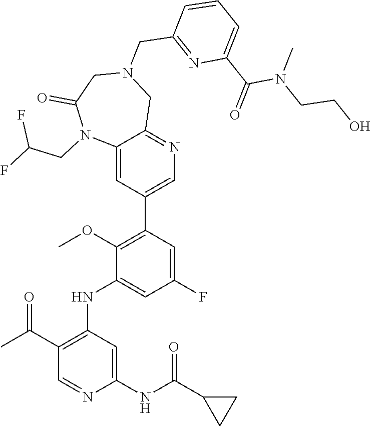 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)CCO)n1)C2] | LCMS calc. for C₃₈H₄₀F₃N₈O₆ [M + H]⁺: m/z = 761.3; Found: 761.4. |
| 70 | 6-((8-(3-((5-acetyl-2- (cyclopropanecarboxamido) pyridin-4-yl)amino)-5- fluoro-2-methoxyphenyl)- 1-(2,2-difluoroethyl)-2- oxo-1,2,3,5-tetrahydro-4H- pyrido[3,2-e][1,4]diazepin- 4-yl)methyl)-N-(tert-butyl)- N-methylpicolinamide | 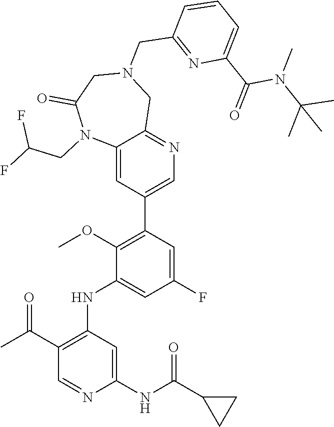 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)C(C)(C)C)n1)C2] | LCMS calc. for C₄₀H₄₄F₃N₈O₅ [M + H]⁺: m/z = 773.3; Found: 773.2. |

### Example 71. 6-((8-(3-((5-acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-pyrido[3,2-e][1,4]diazepin-4-yl)methyl)-N,N-dimethylpicolinamide

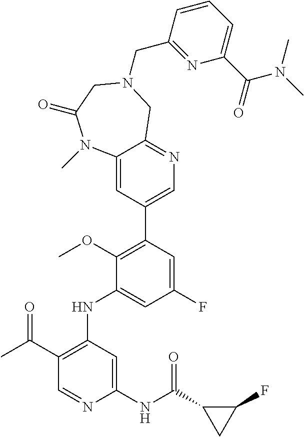 [SMILES: COc1c(Nc2cc(NC(=O)[C@H]3C[C@@H]3F)ncc2C(C)=O)cc(F)cc1-c1cnc2c(c1)N(C)C(=O)CN(Cc1cccc(C(=O)N(C)C)n1)C2]

### Step 1. 6-((Bromo-1-methyl-2-oxo-2,3,5-tetrahydro-4H-pyrido[3,2-e][1,4]diazepin-4-yl)methyl)-N,N-dimethylpicolinamide

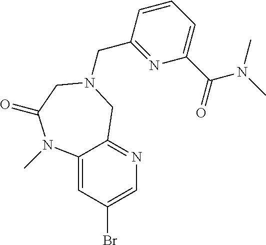 [SMILES: CN(C)C(=O)c1cccc(CN2CC(=O)N(C)c3cc(Br)cnc3C2)n1]

Iodomethane (33 μL, 0.53 mmol) was added to a stirring solution of 6-((8-bromo-2-oxo-1,2,3,5-tetrahydro-4H-pyrido[3,2-e][1,4]diazepin-4-yl)methyl)-N,N-dimethylpicolinamide (Int. 28) (180 mg, 0.45 mmol) and cesium carbonate (290 mg, 0.89 mmol) in DMF (2 mL) at 0° C. The reaction mixture was warmed to rt and stirred for 2 hours. The product mixture was diluted with brine (30 mL) and was extracted with a 3:1 CHCl₃/iPrOH mixture (3×30 mL). The combined organic layers were dried over MgSO₄, filtered and concentrated under reduced pressure. The residue obtained was purified by silica gel column chromatography (0-10% MeOH/DCM) to yield the title compound (163 mg, 87% yield). LC-MS calc. for C₁₈H₂₁BrN₅O₂[M+H]⁺: m/z=418.1/420.1; Found: 418.1/420.1.

### Step 2. 6-((8-(3-((5-Acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-pyrido[3,2-e][1,4]diazepin-4-yl)methyl)-N,N-dimethylpicolinamide

The title compound was prepared in accordance with the synthetic protocols set forth in Example 36 Step 2 using appropriate intermediates, as well as commercial starting materials. LCMS calc. for C₃₆H₃₇F₂N₈O₅[M+H]⁺: m/z=699.3; Found: 699.3. ¹H NMR (400 MHz, DMSO-d₆) δ 11.23 (s, 1H), 11.17 (s, 1H), 8.91 (s, 1H), 8.72 (d, J=1.9 Hz, 1H), 8.16 (d, J=1.9 Hz, 1H), 8.05 (s, 1H), 8.02 (t, J=7.8 Hz, 1H), 7.64 (dd, J=7.8, 1.1 Hz, 1H), 7.55 (dd, J=7.8, 1.0 Hz, 1H), 7.46 (dd, J=9.7, 3.0 Hz, 1H), 7.27 (dd, J=9.0, 3.0 Hz, 1H), 5.01-4.79 (m, 1H), 4.39 (s, 2H), 4.26 (s, 2H), 3.59 (s, 2H), 3.41 (s, 3H), 3.41 (s, 3H), 3.03 (s, 3H), 2.95 (s, 3H), 2.67 (s, 3H), 2.65-2.52 (m, 1H), 1.55 (dddd, J=22.0, 10.1, 6.4, 3.3 Hz, 1H), 1.25 (dq, J=13.0, 6.4 Hz, 1H).

### Examples 72-83

Examples 72-83 are shown below in Table 12 and were prepared in accordance with the synthetic protocols set forth in Example 71 using the appropriate intermediates, as well as commercial starting materials. The following compounds are TFA salts unless otherwise noted.

TABLE 12
Examples 72-83

| | | | Analytical Data |
|---|---|---|---|
| Ex. | Name | Structure | (LCMS and/or NMR) |
| 72 | 6-((8-(3-((5-acetyl-2- (cyclopropanecarboxamido) pyridin-4-yl)amino)-5- fluoro-2-methoxyphenyl)- 1-methyl-2-oxo- 1,2,3,5-tetrahydro- 4H-pyrido[3,2-e][1,4] diazepin-4-yl)methyl)- N,N-dimethylpicolinamide | 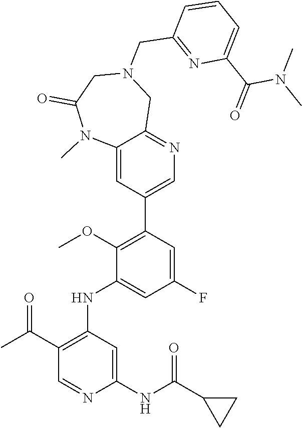 [SMILES: COc1c(Nc2cc(NC(=O)C3CC3)ncc2C(C)=O)cc(F)cc1-c1cnc2c(c1)N(C)C(=O)CN(Cc1cccc(C(=O)N(C)C)n1)C2] | LCMS calc. for C₃₆H₃₈FN₈O₅ [M + H]⁺: m/z = 681.3; Found: 681.3. ¹H NMR (400 MHZ, DMSO-d₆) δ 11.18 (s, 1H), 11.17 (s, 1H), 8.89 (s, 1H), 8.73 (d, J = 1.8 Hz, 1H), 8.17 (d, J = 1.9 Hz, 1H), 8.07-7.99 (m, 2H), 7.65 (dd, J = 7.9, 1.0 Hz, 1H), 7.57 (dd, J = 7.8, 1.0 Hz, 1H), 7.48 (dd, J = 9.7, 3.0 Hz, 1H), 7.29 (dd, J = 9.0, 3.0 Hz, 1H), 4.45 (s, 2H), 4.33 (s, 2H), 3.65 (s, 2H), 3.44-3.39 (m, 6H), 3.03 (s, 3H), 2.95 (s, 3H), 2.67 (s, 3H), 2.03 (ddd, J = 12.5, 7.5, 5.2 Hz, 1H), 0.91- 0.79 (m, 4H). |
| 73 | 6-((8-(3-((5-acetyl- 2-((1R,2S)-2- fluorocyclopropane-1- carboxamido)pyridin- 4-yl)amino)-2- methoxyphenyl)-1- methyl-2-oxo- 1,2,3,5-tetrahydro- 4H-pyrido[3,2-e][1,4] diazepin-4-yl)methyl)- N,N-dimethylpicolinamide | 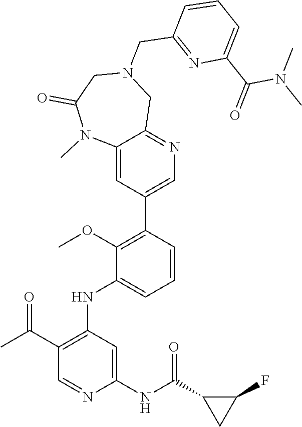 [SMILES: COc1c(Nc2cc(NC(=O)[C@H]3C[C@@H]3F)ncc2C(C)=O)cccc1-c1cnc2c(c1)N(C)C(=O)CN(Cc1cccc(C(=O)N(C)C)n1)C2] | LCMS calc. for C₃₆H₃₈FN₈O₅ [M + H]⁺: m/z = 681.3; Found: 681.4. |
| 74 | 6-((8-(3-((5-acetyl-2- (cyclopropanecarboxamido) pyridin-4-yl)amino)-2- methoxyphenyl)-1- methyl-2-oxo- 1,2,3,5-tetrahydro- 4H-pyrido[3,2-e][1,4] diazepin-4-yl)methyl)- N,N-dimethylpicolinamide | 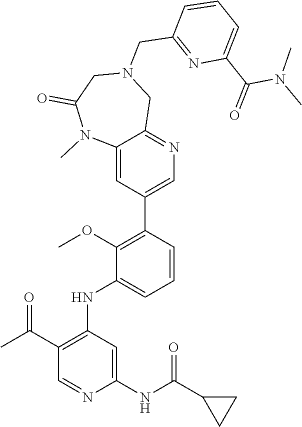 [SMILES: COc1c(Nc2cc(NC(=O)C3CC3)ncc2C(C)=O)cccc1-c1cnc2c(c1)N(C)C(=O)CN(Cc1cccc(C(=O)N(C)C)n1)C2] | LCMS calc. for C₃₆H₃₉N₈O₅ [M + H]⁺: m/z = 663.3; Found: 663.4. |
| 75 | 6-((8-(3-((5-acetyl- 2-((1R,2S)-2- fluorocyclopropane-1- carboxamido)pyridin-4- yl)amino)-2- methoxyphenyl)-6- fluoro-1-methyl-2- oxo-1,2,3,5- tetrahydro-4H- benzo[e][1,4]diazepin- 4-yl)methyl)-N,N- dimethylpicolinamide | 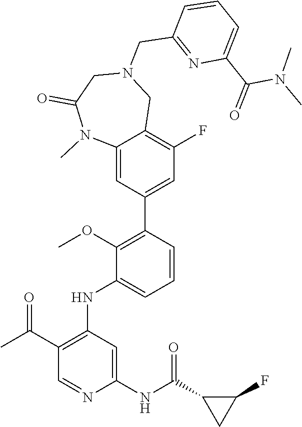 [SMILES: COc1c(Nc2cc(NC(=O)[C@H]3C[C@@H]3F)ncc2C(C)=O)cccc1-c1cc(F)c2c(c1)N(C)C(=O)CN(Cc1cccc(C(=O)N(C)C)n1)C2] | LCMS calc. for C₃₇H₃₈F₂N₇O₅ [M + H]⁺: m/z = 698.3; Found: 698.3. |
| 76 | 6-((8-(3-((5-acetyl-2- (cyclopropanecarbox amido)pyridin-4- yl)amino)-5-fluoro- 2-methoxyphenyl)- 6-fluoro-1-methyl-2- oxo-1,2,3,5- tetrahydro-4H- benzo[e][1,4]diazepin- 4-yl)methyl)-N,N- dimethylpicolinamide | 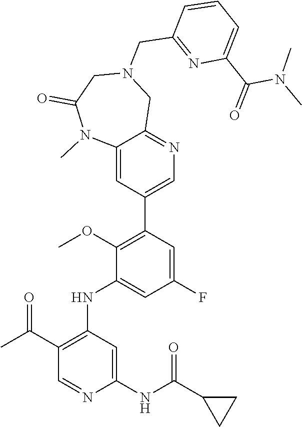 [SMILES: COc1c(Nc2cc(NC(=O)C3CC3)ncc2C(C)=O)cc(F)cc1-c1cnc2c(c1)N(C)C(=O)CN(Cc1cccc(C(=O)N(C)C)n1)C2] | LCMS calc. for C₃₇H₃₈F₂N₇O₅ [M + H]⁺: m/z = 698.3; Found: 698.3. |
| 77 | 6-((8-(3-((5-acetyl-2- (cyclopropanecarboxamido) pyridin-4-yl)amino)-2- methoxyphenyl)-6- fluoro-1-methyl-2- oxo-1,2,3,5- tetrahydro-4H- benzo[e][1,4]diazepin- 4-yl)methyl)-N,N- dimethylpicolinamide | 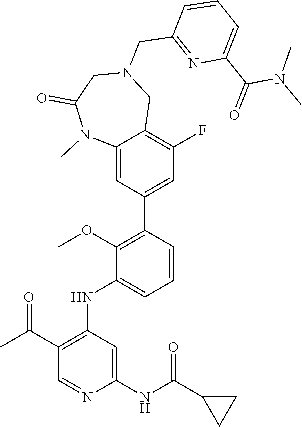 [SMILES: COc1c(Nc2cc(NC(=O)C3CC3)ncc2C(C)=O)cccc1-c1cc(F)c2c(c1)N(C)C(=O)CN(Cc1cccc(C(=O)N(C)C)n1)C2] | LCMS calc. for C37H39FN7O5 [M + H]⁺: m/z = 680.3; Found: 680.3. |
| 78 | 6-((8-(3-((5-acetyl- 2-((1R,2S)-2- fluorocyclopropane-1- carboxamido)pyridin- 4-yl)amino)-5-fluoro-2- methoxyphenyl)-1- methyl-2-oxo- 1,2,3,5-tetrahydro-4H- benzo[e][1,4]diazepin- 4-yl)methyl)-N,N- dimethylpicolinamide | 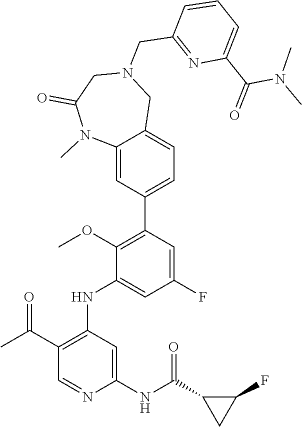 [SMILES: COc1c(Nc2cc(NC(=O)[C@H]3C[C@@H]3F)ncc2C(C)=O)cc(F)cc1-c1ccc2c(c1)N(C)C(=O)CN(Cc1cccc(C(=O)N(C)C)n1)C2] | LCMS calc. for C₃₇H₃₈F₂N₇O₅ [M + H]⁺: m/z = 698.3; Found: 698.5. |
| 79 | 6-((8-(3-((5-acetyl- 2-((1S,2R)-2- fluorocyclopropane-1- carboxamido)pyridin- 4-yl)amino)-5- fluoro-2- methoxyphenyl)-1- methyl-2-oxo- 1,2,3,5-tetrahydro-4H- benzo[e][1,4]diazepin- 4-yl)methyl)-N,N- dimethylpicolinamide | 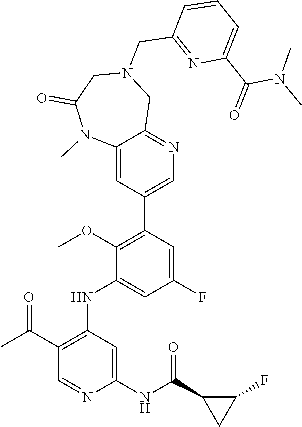 [SMILES: COc1c(Nc2cc(NC(=O)[C@@H]3C[C@H]3F)ncc2C(C)=O)cc(F)cc1-c1cnc2c(c1)N(C)C(=O)CN(Cc1cccc(C(=O)N(C)C)n1)C2] | LCMS calc. for C₃₇H₃₈F₂N₇O₅ [M + H]⁺: m/z = 698.3; Found: 698.5. |
| 80 | 6-((8-(3-((5-acetyl-2- (cyclopropanecarboxamido) pyridin-4- yl)amino)-5-fluoro- 2-methoxyphenyl)- 1-methyl-2-oxo- 1,2,3,5-tetrahydro-4H- benzo[e][1,4]diazepin- 4-yl)methyl)-N,N- dimethylpicolinamide | 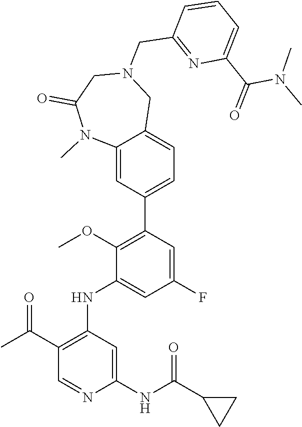 [SMILES: COc1c(Nc2cc(NC(=O)C3CC3)ncc2C(C)=O)cc(F)cc1-c1ccc2c(c1)N(C)C(=O)CN(Cc1cccc(C(=O)N(C)C)n1)C2] | LCMS calc. for C₃₇H₃₉FN₇O₅ [M + H]⁺: m/z = 680.3; Found: 680.6. |
| 81 | 6-((8-(3-((5-acetyl-2- (cyclopropanecarboxamido) pyridin-4-yl)amino)-2- methoxy-5- methylphenyl)-1- methyl-2-oxo- 1,2,3,5-tetrahydro-4H- benzo[e][1,4]diazepin- 4-yl)methyl)-N,N- dimethylpicolinamide | 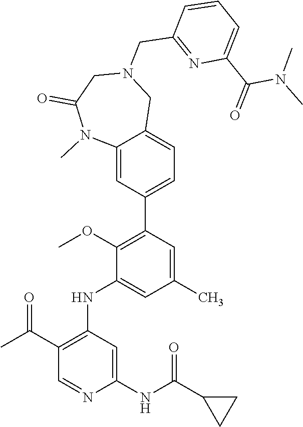 [SMILES: COc1c(Nc2cc(NC(=O)C3CC3)ncc2C(C)=O)cc(C)cc1-c1ccc2c(c1)N(C)C(=O)CN(Cc1cccc(C(=O)N(C)C)n1)C2] | LCMS calc. for C₃₈H₄₂N₇O₅ [M + H]⁺: m/z = 676.3; Found: 676.4. |
| 82 | 6-((8-(3-((5-acetyl-2- (cyclopropanecarboxamido) pyridin-4-yl)amino)-2- methoxy-5- (trifluoromethyl) phenyl)-1-methyl-2- oxo-1,2,3,5- tetrahydro-4H- benzo[e][1,4]diazepin- 4-yl)methyl)-N,N- dimethylpicolinamide | 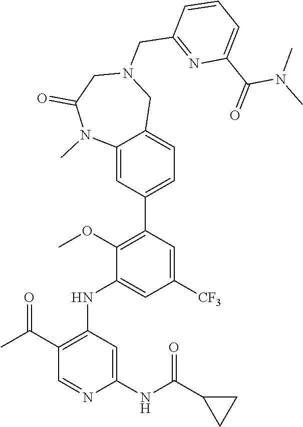 [SMILES: COc1c(Nc2cc(NC(=O)C3CC3)ncc2C(C)=O)cc(C(F)(F)F)cc1-c1ccc2c(c1)N(C)C(=O)CN(Cc1cccc(C(=O)N(C)C)n1)C2] | LCMS calc. for C₃₈H₃₉F₃N₇O₅ [M + H]⁺: m/z = 730.3; Found: 730.5. |
| 83 | 6-((8-(3-((5-acetyl-2- (cyclopropanecarboxamido) pyridin-4-yl)amino)-4- fluoro-2-methoxyphenyl)- 1-methyl-2-oxo- 1,2,3,5-tetrahydro-4H- benzo[e][1,4]diazepin- 4-yl)methyl)-N,N- dimethylpicolinamide | 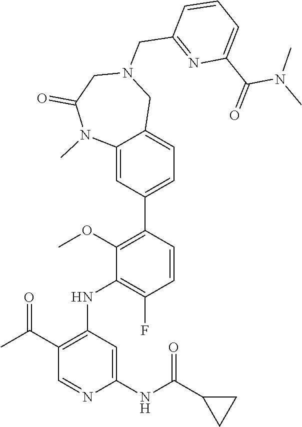 [SMILES: COc1c(-c2ccc3c(c2)N(C)C(=O)CN(Cc2cccc(C(=O)N(C)C)n2)C3)ccc(F)c1Nc1cc(NC(=O)C2CC2)ncc1C(C)=O] | LCMS calc. for C₃₇H₃₉FN₇O₅ [M + H]⁺: m/z = 680.3; Found 680.4. |

### Example 84. 6-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

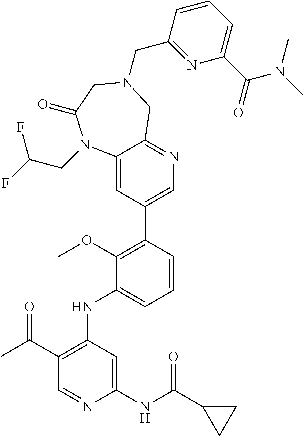 [SMILES: COc1c(Nc2cc(NC(=O)C3CC3)ncc2C(C)=O)cccc1-c1cnc2c(c1)N(CC(F)F)C(=O)CN(Cc1cccc(C(=O)N(C)C)n1)C2]

### Step 1. (3-((5-Acetyl-2-(cyclopropanecarboxamido)pyridin-4-yl)amino)-2-methoxyphenyl)boronic acid

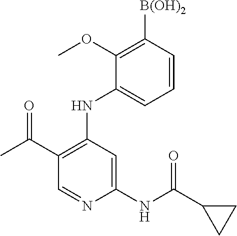 [SMILES: COc1c(Nc2cc(NC(=O)C3CC3)ncc2C(C)=O)cccc1B(O)O]

Tetrahydroxydiboron (2.13 g, 23.7 mmol) was added to a stirring solution of N-(5-acetyl-4-((3-bromo-2-methoxyphenyl)amino)pyridin-2-yl)cyclopropanecarboxamide (Int. 3) (3.2 g, 7.9 mmol), potassium acetate (2.33 g, 23.7 mmol), and 2-dicyclohexylphosphino-2′,4′,6′-triisopropyl-biphenyl (377 mg, 0.79 mmol) in THF (30 mL), EtOH (30 mL), and ethylene glycol (10 mL). The reaction mixture was purged with nitrogen gas three times. Then chloro(2-dicyclohexylphosphino-2′,4′,6′-triisopropyl-1,1′-biphenyl)[2-(2′-amino-1,1′-biphenyl)]palladium(II) (622 mg, 0.79 mmol) was added to the reaction mixture under N₂. The reaction mixture was heated to 50° C. for 2 hours. The product mixture was diluted with water (100 mL) and extracted with DCM (3×100 mL). The combined organic layers were washed with brine (300 mL). The separated organic layer was concentrated under reduced pressure. The residue obtained was purified by silica gel column chromatography (eluting with 1:1:1 EtOAc/DCM/Hexane) to yield the title compound (3.5 g, quant. yield). LC-MS calc. for C₁₈H₂₁BN₃O₅[M+H]⁺: m/z=370.2; Found: 370.0.

### Step 2. 6-((8-(3-((5-acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

A 20 mL scintillation vial was charged with 6-((8-bromo-1-(2,2-difluoroethyl)-2-oxo-1,2,3,5-tetrahydro-4H-pyrido[3,2-e][1,4]diazepin-4-yl)methyl)-N,N-dimethylpicolinamide (53 mg, 0.11 mmol), (3-((5-acetyl-2-(cyclopropanecarboxamido)pyridin-4-yl)amino)-2-methoxyphenyl)-boronic acid (63 mg, 0.17 mmol), potassium carbonate (47 mg, 0.34 mmol), and [1,1′-bis(diphenyl-phosphino)ferrocene]dichloropalladium(II) complex with dichloromethane (9.3 mg, 0.011 mmol). The mixture was dissolved in 1,4-dioxane (1 mL) and water (200 μL), was sparged with nitrogen gas for 5 minutes and sealed. Then the reaction mixture was heated to 80° C. for 4 hours. The product mixture was diluted with MeCN (6 mL), filtered, and purified directly by prep-HPLC on a C₁₈ column (16.7-36.7% MeCN in 0.2% TFA(aq)) to afford the TFA salt of the title compound (54 mg, 51% yield). LC-MS calc. for C₃₇H₃₉F₂N₈O₅[M+H]⁺: m/z=713.3; Found: 713.3.

### Examples 85-102

Examples 85-102 are shown below in Table 13 and were prepared in accordance with the synthetic protocols set forth in Example 84 using the appropriate intermediates, as well as commercial starting materials. The following compounds are TFA salts unless otherwise noted.

TABLE 13
Examples 85-102

| | | | Analytical Data |
|---|---|---|---|
| Ex. | Name | Structure | (LCMS and/or NMR) |
| 85 | (R)-6-((8-(3-((5- acetyl-2- (cyclopropanecarboxamido) pyridin-4-yl)amino)-2- methoxyphenyl)-2- oxo-1- (tetrahydrofuran-3- yl)-1,2,3,5- tetrahydro-4H-pyrido [3,2-e][1,4]diazepin-4- yl)methyl)-N,N- dimethylpicolinamide | 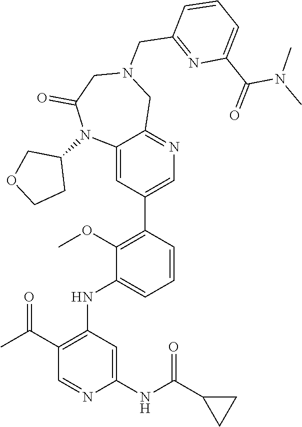 [SMILES: COc1c(Nc2cc(NC(=O)C3CC3)ncc2C(C)=O)cccc1-c1cnc2c(c1)N([C@@H]1CCOC1)C(=O)CN(Cc1cccc(C(=O)N(C)C)n1)C2] | LC-MS calc. for C₃₉H₄₃N₈O₆ [M + H]⁺: m/z = 719.3; Found: 719.3. |
| 86 | (S)-6-((8-(3-((5-acetyl-2- (cyclopropanecarbox amido)pyridin-4- yl)amino)-2- methoxyphenyl)-2- oxo-1-(tetrahydrofuran-3- yl)-1,2,3,5- tetrahydro-4H- pyrido[3,2-e][1,4] diazepin-4-yl)methyl)- N,N-dimethylpicolinamide | 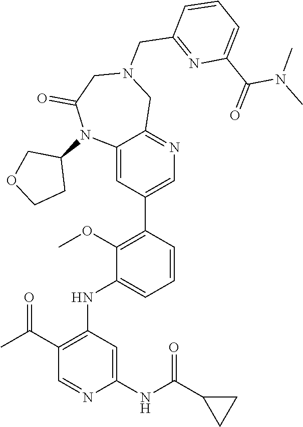 [SMILES: COc1c(Nc2cc(NC(=O)C3CC3)ncc2C(C)=O)cccc1-c1cnc2c(c1)N([C@H]1CCOC1)C(=O)CN(Cc1cccc(C(=O)N(C)C)n1)C2] | LC-MS calc. for C₃₉H₄₃N₈O₆ [M + H]⁺: m/z = 719.3; Found: 719.3. |
| 87 | 6-((8-(3-((5-acetyl-2- (cyclopropanecarboxamido) pyridin-4- yl)amino)-2- methoxyphenyl)-1- isopropyl-2-oxo- 1,2,3,5-tetrahydro- 4H-pyrido[3,2-e] [1,4]diazepin-4- yl)methyl)-N,N- dimethylpicolinamide | 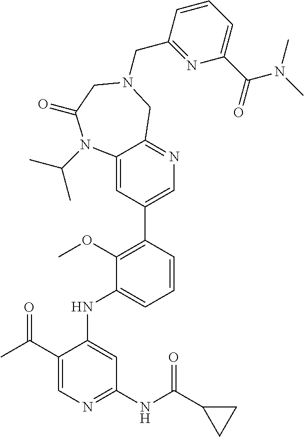 [SMILES: COc1c(Nc2cc(NC(=O)C3CC3)ncc2C(C)=O)cccc1-c1cnc2c(c1)N(C(C)C)C(=O)CN(Cc1cccc(C(=O)N(C)C)n1)C2] | LC-MS calc. for C₃₈H₄₃N₈O₅ [M + H]⁺: m/z= 691.3; Found: 691.4. |
| 88 | 6-((8-(3-((5-acetyl-2- (cyclopropanecarboxamido) pyridin-4- yl)amino)-2- methoxyphenyl)-1- ethyl-2-oxo-1,2,3,5- tetrahydro-4H- pyrido[3,2-e][1,4] diazepin-4- yl)methyl)-N,N- dimethylpicolinamide | 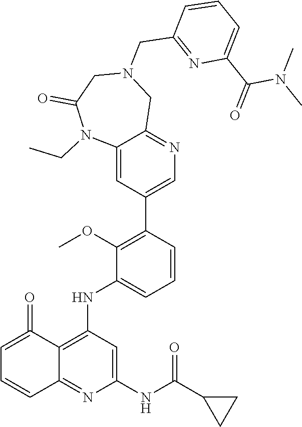 | LC-MS calc. for C₃₇H₄₁N₈O₅ [M + H]⁺: m/z = 677.3; Found: 677.3. |
| 89 | 6-((8-(3-((5-acetyl-2- (cyclopropanecarboxamido) pyridin-4- yl)amino)-5-fluoro-2- methoxyphenyl)-1- isopropyl-2-oxo- 1,2,3,5-tetrahydro- 4H-pyrido[3,2-e][1,4] diazepin-4- yl)methyl)-N,N- dimethylpicolinamide | 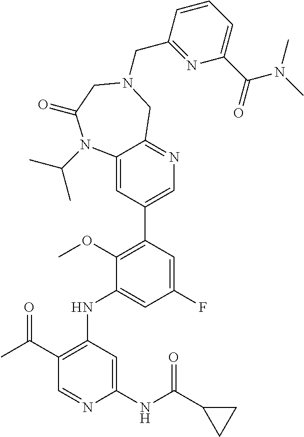 [SMILES: COc1c(Nc2cc(NC(=O)C3CC3)ncc2C(C)=O)cc(F)cc1-c1cnc2c(c1)N(C(C)C)C(=O)CN(Cc1cccc(C(=O)N(C)C)n1)C2] | LC-MS calc. for C₃₈H₄₂FN₈O₅ [M + H]⁺: m/z = 709.3; Found: 709.5. |
| 90 | 6-((8-(3-((5-acetyl-2- (cyclopropanecarboxamido) pyridin-4-yl)amino)-2- methoxyphenyl)-1- ((1s,3s)-3- cyanocyclobutyl)-2- oxo-1,2,3,5- tetrahydro-4H- benzo[e][1,4]diazepin-4- yl)methyl)-N,N- dimethylpicolinamide | 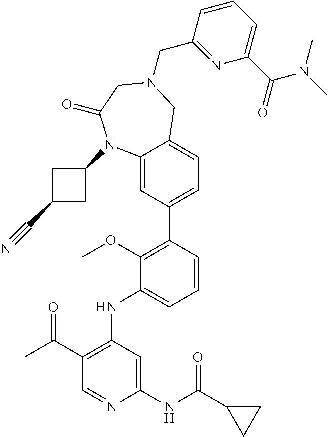 [SMILES: COc1c(Nc2cc(NC(=O)C3CC3)ncc2C(C)=O)cccc1-c1ccc2c(c1)N([C@H]1C[C@@H](C#N)C1)C(=O)CN(Cc1cccc(C(=O)N(C)C)n1)C2] | LC-MS calc. for C₄₁H₄₃N₈O₅ [M + H]⁺: m/z = 727.3; Found: 727.5. |
| 91 | 6-((8-(3-((5-acetyl-2- (cyclopropanecarboxamido) pyridin-4-yl)amino)-2- methoxyphenyl)-1- (1-cyanoethyl)-2- oxo-1,2,3,5- tetrahydro-4H- benzo[e][1,4]diazepin-4- yl)methyl)-N,N- dimethylpicolinamide | 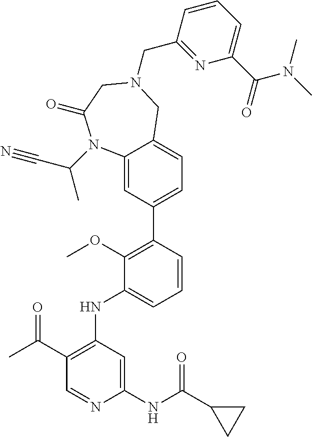 [SMILES: COc1c(Nc2cc(NC(=O)C3CC3)ncc2C(C)=O)cccc1-c1ccc2c(c1)N(C(C)C#N)C(=O)CN(Cc1cccc(C(=O)N(C)C)n1)C2] | LC-MS calc. for C₃₉H₄₁N₈O₅ [M + H]⁺: m/z = 701.3; Found: 701.6. |
| 92 | 6-((8-(3-((5-acetyl-2- (cyclopropanecarboxamido) pyridin-4-yl)amino)-2- methoxy-5- (trifluoromethyl)phen yl)-1-(2,2- difluoroethyl)-2-oxo- 1,2,3,5-tetrahydro- 4H-pyrido[3,2-e] [1,4]diazepin-4- yl)methyl)-N,N- dimethylpicolinamide | 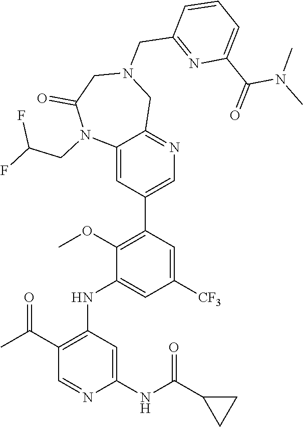 [SMILES: COc1c(Nc2cc(NC(=O)C3CC3)ncc2C(C)=O)cc(C(F)(F)F)cc1-c1cnc2c(c1)N(CC(F)F)C(=O)CN(Cc1cccc(C(=O)N(C)C)n1)C2] | LC-MS calc. for C₃₈H₃₈F₅N₈O₅ [M + H]⁺: m/z = 781.3; Found: 781.4. ¹H NMR (600 MHZ, DMSO-d₆) δ 11.12 (s, 1H), 11.01 (s, 1H), 8.89 (s, 1H), 8.67 (s, 1H), 8.19 (s, 1H), 8.04 (s, 1H), 7.94 (t, J = 7.8 Hz, 1H), 7.89 (s, 1H), 7.70 (s, 1H), 7.57 (d, J = 7.6 Hz, 1H), 7.46 (d, J = 7.7 Hz, 1H), 6.31 (s, 1H), 4.41 (t, J = 15.5 Hz, 2H), 3.94 (s, 2H), 3.85 (s, 2H), 3.43 (s, 3H), 3.21 (s, 2H), 3.01 (s, 3H), 2.94 (s, 3H), 2.66 (s, 3H), 2.06- 1.98 (m, 1H), 0.85-0.73 (m, 4H). |
| 93 | 6-((8-(3-((5-acetyl-2- (cyclopropanecarboxamido) pyridin-4-yl)amino)-2- methoxyphenyl)-1- ethyl-7-fluoro-2-oxo- 1,2,3,5-tetrahydro-4H- benzo[e][1,4]diazepin- 4-yl)methyl)-N,N- dimethylpicolinamide | 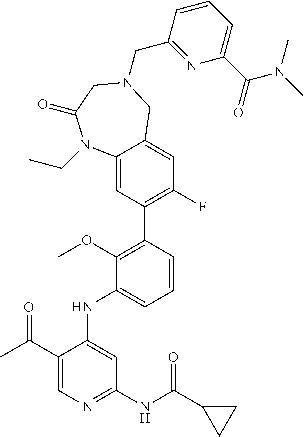 [SMILES: CCN1C(=O)CN(Cc2cccc(C(=O)N(C)C)n2)Cc2cc(F)c(-c3cccc(Nc4cc(NC(=O)C5CC5)ncc4C(C)=O)c3OC)cc21] | LC-MS calc. for C₃₈H₄₁FN₇O₅ [M + H]⁺: m/z = 694.3; Found: 694.4. |
| 94 | 6-((8-(3-((5-acetyl-2- (cyclopropanecarboxamido) pyridin-4-yl)amino)-2- methoxyphenyl)-1- (2-cyanoethyl)-2- oxo-1,2,3,5- tetrahydro-4H- benzo[e][1,4]diazepin- 4-yl)methyl)-N,N- dimethylpicolinamide | 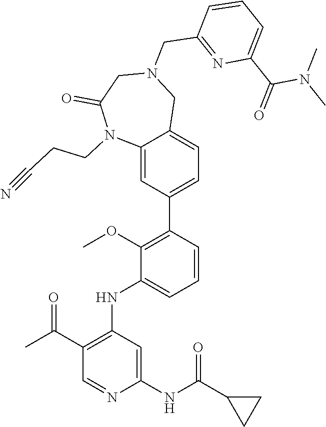 [SMILES: COc1c(Nc2cc(NC(=O)C3CC3)ncc2C(C)=O)cccc1-c1ccc2c(c1)N(CCC#N)C(=O)CN(Cc1cccc(C(=O)N(C)C)n1)C2] | LCMS calc. for C₃₉H₄₁N₈O₅ [M + H]⁺: m/z = 701.3; Found: 701.4. |
| 95 | 6-((8-(3-((5-acetyl-2- (cyclopropanecarboxamido) pyridin-4-yl)amino)-2- methoxyphenyl)-1- methyl-2-oxo-1,2,3,5- tetrahydro-4H- pyrido[4,3-e][1,4] diazepin-4- yl)methyl)-N,N- dimethylpicolinamide | 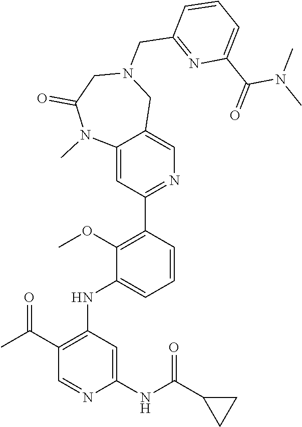 [SMILES: COc1c(Nc2cc(NC(=O)C3CC3)ncc2C(C)=O)cccc1-c1cc2c(cn1)CN(Cc1cccc(C(=O)N(C)C)n1)CC(=O)N2C] | LCMS calc. for C₃₆H₃₉N₈O₅ [M + H]⁺: m/z = 663.30; Found: 663.35. |
| 96 | 6-((8-(3-((5-acetyl-2- ((1S,2R)-2- fluorocyclopropane-1- carboxamido)pyridin- 4-yl)amino)-2- methoxyphenyl)-1- (2-methoxyethyl)-2- oxo-1,2,3,5- tetrahydro-4H- pyrido[4,3-e][1,4] diazepin-4- yl)methyl)-N,N- dimethylpicolinamide | 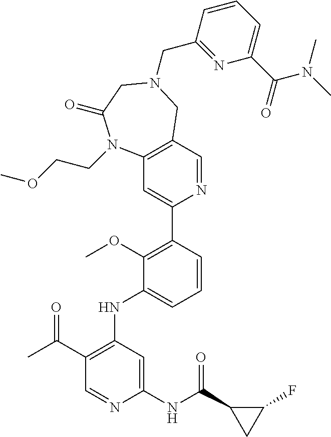 | LCMS calc. for C₃₈H₄₂FN₈O₆ [M + H]⁺: m/z = 725.3; Found: 725.2. |
| 97 | 6-((8-(3-((5-acetyl-2- ((1R,2S)-2- fluorocyclopropane-1- carboxamido)pyridin- 4-yl)amino)-5-fluoro- 2-methoxyphenyl)-1- methyl-2-oxo-1,2,3,5- tetrahydro-4H-pyrido[4,3- e][1,4]diazepin-4- yl)methyl)-N,N- dimethylpicolinamide | 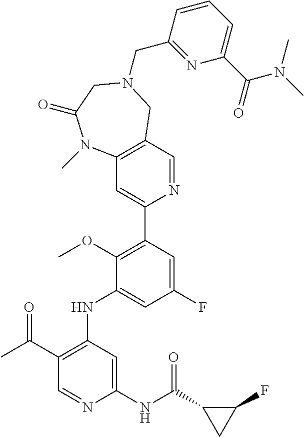 [SMILES: COc1c(Nc2cc(NC(=O)[C@H]3C[C@@H]3F)ncc2C(C)=O)cc(F)cc1-c1cc2c(cn1)CN(Cc1cccc(C(=O)N(C)C)n1)CC(=O)N2C] | LCMS calc. for C₃₆H₃₇F₂N₈O₅ [M + H]⁺: m/z = 699.3; Found: 699.2. |
| 98 | 6-((8-(3-((5-acetyl-2- (cyclopropanecarboxamido) pyridin-4-yl)amino)-2- methoxyphenyl)-1- isopropyl-2-oxo- 1,2,3,5-tetrahydro- 4H-pyrido[4,3-e] [1,4]diazepin-4- yl)methyl)-N,N- dimethylpicolinamide | 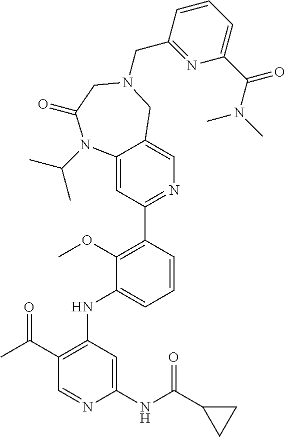 [SMILES: COc1c(Nc2cc(NC(=O)C3CC3)ncc2C(C)=O)cccc1-c1cc2c(cn1)CN(Cc1cccc(C(=O)N(C)C)n1)CC(=O)N2C(C)C] | LCMS calc. for C₃₈H₄₃N₈O₅ [M + H]⁺: m/z = 691.3; Found 691.2. |
| 99 | 6-((8-(3-((5-acetyl-2- (cyclopropanecarboxamido) pyridin-4- yl)amino)-5-fluoro-2- methoxyphenyl)-1- (2,2-difluoroethyl)-2- oxo-1,2,3,5- tetrahydro-4H- pyrido[4,3-e][1,4] diazepin-4-yl)methyl)- N,N-dimethylpicolinamide | 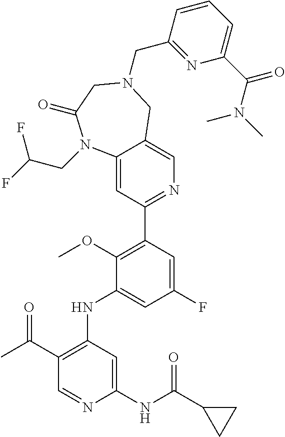 [SMILES: COc1c(Nc2cc(NC(=O)C3CC3)ncc2C(C)=O)cc(F)cc1-c1cc2c(cn1)CN(Cc1cccc(C(=O)N(C)C)n1)CC(=O)N2CC(F)F] | LCMS calc. for C₃₇H₃₈F₃N₈O₅ [M + H]⁺: m/z = 731.3; Found 731.2. |
| 100 | 6-((8-(3-((5-acetyl-2- (cyclopropanecarbox amido)pyridin-4- yl)amino)-2- methoxyphenyl)-1- (2,2-difluoropropyl)- 2-oxo-1,2,3,5- tetrahydro-4H- pyrido[3,2-e][1,4] diazepin-4-yl)methyl)-N,N- dimethylpicolinamide | 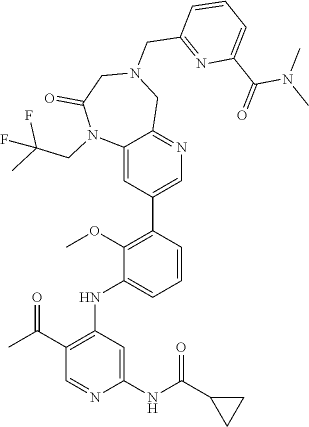 [SMILES: COc1c(Nc2cc(NC(=O)C3CC3)ncc2C(C)=O)cccc1-c1cnc2c(c1)N(CC(C)(F)F)C(=O)CN(Cc1cccc(C(=O)N(C)C)n1)C2] | LC-MS calc. for C₃₈H₄₁F₂N₈O₅ [M + H]⁺: m/z = 727.3; Found: 727.3. |
| 101 | (R)-6-((8-(3-((5-acetyl-2- (cyclopropanecarboxamido) pyridin-4-yl)amino)-2- methoxyphenyl)-1- (1-methoxypropan-2- yl)-2-oxo-1,2,3,5- tetrahydro-4H- pyrido[3,2-e][1,4] diazepin-4- yl)methyl)-N,N- dimethylpicolinamide | 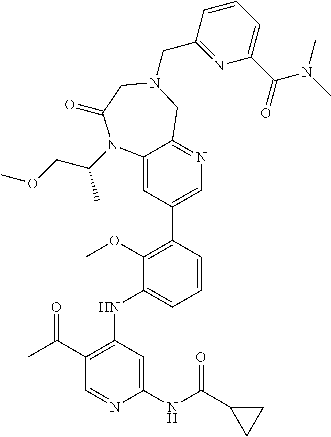 | LC-MS calc. for C₃₉H₄₅N₈O₆ [M + H]⁺: m/z = 721.3; Found: 721.2. |
| 102 | (S)-6-((8-(3-((5-acetyl-2- (cyclopropanecarbox amido)pyridin-4- yl)amino)-2- methoxyphenyl)-2-oxo-1- (tetrahydrofuran-3- yl)-1,2,3,5- tetrahydro-4H- benzo[e][1,4]diazepin- 4-yl)methyl)-N,N- dimethylpicolinamide | 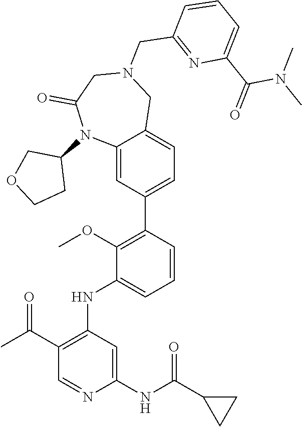 [SMILES: COc1c(Nc2cc(NC(=O)C3CC3)ncc2C(C)=O)cccc1-c1ccc2c(c1)N([C@H]1CCOC1)C(=O)CN(Cc1cccc(C(=O)N(C)C)n1)C2] | LC-MS calc. for C₄₀H₄₄N₇O₆ [M + H]⁺: m/z = 718.3; Found: 718.2. ¹H NMR (400 MHz, CDCl₃) δ 12.79 (s, 1H), 11.59 (s, 1H), 8.61 (s, 1H), 8.26 (s, 1H), 7.92 (t, J = 7.8 Hz, 1H), 7.68-7.55 (m, 5H), 7.48 (d, J = 7.7 Hz, 1H), 7.35 (q, J = 6.2 Hz, 2H), 5.09 (d, J = 117.7 Hz, 1H), 4.74-4.53 (m, 3H), 4.09 (s, 3H), 3.81 (s, 3H), 3.40 (s, 3H), 3.15 (s, 3H), 3.04 (s, 3H), 2.67 (s, 3H), 2.51 (s, 2H), 1.99 (dt, J = 7.8, 4.9 Hz, 1H), 1.06 (s, 2H), 0.99 (dd, J = 7.8, 3.5 Hz, 2H). |

### Example 103. 6-((8-(4-((5-Acetyl-2-(cyclopropanecarboxamido)pyridin-4-yl)amino)-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide

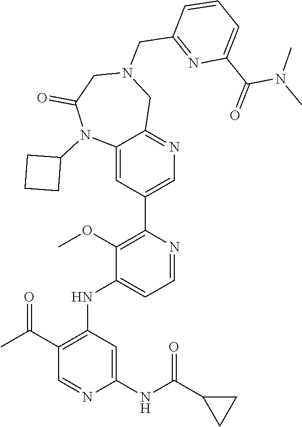 [SMILES: COc1c(Nc2cc(NC(=O)C3CC3)ncc2C(C)=O)ccnc1-c1cnc2c(c1)N(C1CCC1)C(=O)CN(Cc1cccc(C(=O)N(C)C)n1)C2]

### Step 1. 6-((8-Bromo-1-cyclobutyl-2-oxo-1,2,3,5-tetrahydro-4H-pyrido[3,2-e][1,4]diazepin-4-yl)methyl)-N,N-dimethylpicolinamide

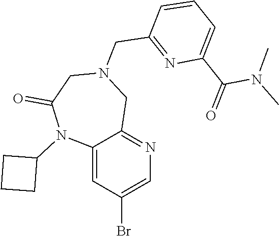 [SMILES: CN(C)C(=O)c1cccc(CN2CC(=O)N(C3CCC3)c3cc(Br)cnc3C2)n1]

To a stirring solution of 6-((8-bromo-2-oxo-1,2,3,5-tetrahydro-4H-pyrido[3,2-e][1,4]diazepin-4-yl)methyl)-N,N-dimethylpicolinamide (Int. 28) (200 mg, 0.50 mmol) in DMF (3 mL) was added cesium carbonate (484 mg, 1.48 mmol) and iodocyclobutane (237 μL, 2.47 mmol) sequentially. The reaction mixture was heated to 60° C. and stirred for 2 hours. The product mixture was diluted with brine (30 mL) and extracted with a 3:1 CHCl₃/iPrOH mixture (3×30 mL). The combined organic layers were dried over MgSO₄, filtered and concentrated under reduced pressure. The residue obtained was purified by silica gel column chromatography (0-5% MeOH/DCM) to yield the title compound (158 mg, 70% yield). LC-MS calc. for C₂₁H₂₅BrN₅O₂[M+H]⁺: m/z=458.1/460.1; Found: 458.1/460.1.

### Step 2. 6-((8-(4-amino-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide

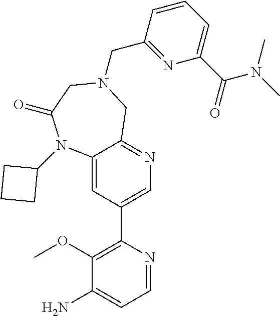 [SMILES: COc1c(N)ccnc1-c1cnc2c(c1)N(C1CCC1)C(=O)CN(Cc1cccc(C(=O)N(C)C)n1)C2]

A 20 mL scintillation vial was charged with bis(pinacolato)diboron (158 mg, 0.62 mmol), potassium pivalate (145 mg, 1.0 mmol), [1,1′-bis(diphenylphosphino)ferrocene]dichloro-palladium(II) complex with dichloromethane (28 mg, 0.034 mmol), and 6-((8-bromo-1-cyclobutyl-2-oxo-1,2,3,5-tetrahydro-4H-pyrido[3,2-e][1,4]diazepin-4-yl)methyl)-N,N-dimethylpicolinamide (158 mg, 0.35 mmol). The mixture was dissolved in 1,4-dioxane (3 mL), sparged with nitrogen gas for 5 minutes and sealed. Then the reaction mixture was heated to 95° C. for 50 minutes. The mixture was cooled to rt, and 2-chloro-3-methoxypyridin-4-amine (164 mg, 1.0 mmol), potassium carbonate (143 mg, 1.0 mmol), [1,1′-bis(diphenylphosphino)ferrocene]dichloropalladium(II) complex with dichloromethane (28 mg, 0.034 mmol), and water (600 μL) were added to the reaction mixture sequentially. The mixture was sparged with nitrogen gas for 5 minutes and sealed. The reaction mixture was heated to 100° C. and stirred overnight. The product mixture was diluted with brine (60 mL) and extracted with a 3:1 CHCl₃/iPrOH mixture (3×60 mL). The combined organic layers were dried over MgSO₄, filtered and concentrated under reduced pressure. The residue obtained was purified by silica gel column chromatography (0-15% MeOH/DCM) to yield the title compound (156 mg, 90% yield). LC-MS calc. for C₂₇H₃₂N₇O₃ [M+H]⁺: m/z=502.3; Found: 502.2.

### Step 3. 6-((8-(4-((5-acetyl-2-(cyclopropanecarboxamido)pyridin-4-yl)amino)-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide

A 20 mL scintillation vial was charged with 6-((8-(4-amino-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide (156 mg, 0.31 mmol), N-(5-acetyl-4-chloropyridin-2-yl)cyclopropanecarboxamide (Int. 17) (111 mg, 0.47 mmol), cesium carbonate (304 mg, 0.93 mmol), tris(dibenzylideneacetone)dipalladium(0) (29 mg, 0.031 mmol), and 1,1′-bis(diphenylphosphino)-ferrocene (35 mg, 0.062 mmol). The mixture was dissolved in 1,4-dioxane (2 mL), sparged with nitrogen gas for 5 minutes and sealed. The reaction mixture was heated to 100° C. and stirred overnight. The product mixture was diluted with MeCN (6 mL), filtered, and purified directly by prep-HPLC on a C₁₈ column (10.5-28.5% MeCN in 0.2% TFA(aq)) to afford the TFA salt of the title compound (48 mg, 17% yield). LC-MS calc. for C₃₈H₄₂N₉O₅ [M+H]⁺: m/z=704.3; Found: 704.3.

### Examples 104-134

Examples 104-134 are shown below in Table 14 and were prepared in accordance with the synthetic protocols set forth in Example 103 using the appropriate intermediates, as well as commercial starting materials. The following compounds are TFA salts unless otherwise noted.

TABLE 14
Examples 104-134

| | | | Analytical Data (LCMS |
|---|---|---|---|
| Ex. | Name | Structure | and/or NMR) |
| 104 | (S)-6-((8-(4-((5-acetyl- 2- (cyclopropanecarboxamido) pyridin-4- yl)amino)-3- methoxypyridin-2-yl)- 6-fluoro-2-oxo-1- (tetrahydrofuran-3-yl)- 1,2,3,5-tetrahydro-4H- benzo[e][1,4]diazepin- 4-yl)methyl)-N,N- dimethylpicolinamide | 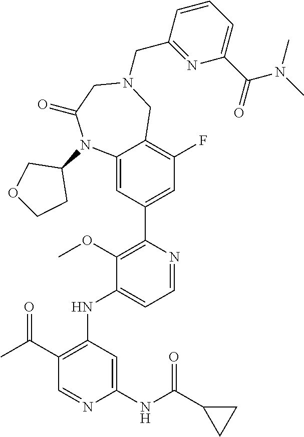 [SMILES: COc1c(Nc2cc(NC(=O)C3CC3)ncc2C(C)=O)ccnc1-c1cc(F)c2c(c1)N([C@H]1CCOC1)C(=O)CN(Cc1cccc(C(=O)N(C)C)n1)C2] | LC-MS calc. for C₃₉H₄₂FN₈O₆ [M + H]⁺: m/z = 737.3; Found: 737.3. |
| 105 | 6-((8-(4-((5-acetyl-2- (cyclopropanecarboxamido) pyridin-4-yl)amino)-3- methoxypyridin-2-yl)- 1-(2,2-difluoroethyl)- 6-fluoro-2-oxo- 1,2,3,5-tetrahydro-4H- benzo[e][1,4]diazepin- 4-yl)methyl)-N,N- dimethylpicolinamide | 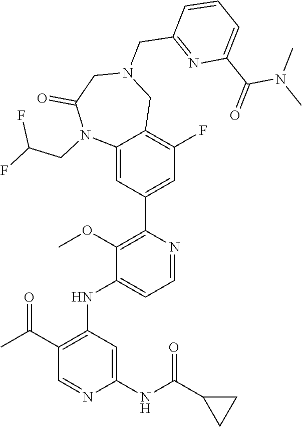 [SMILES: COc1c(Nc2cc(NC(=O)C3CC3)ncc2C(C)=O)ccnc1-c1cc(F)c2c(c1)N(CC(F)F)C(=O)CN(Cc1cccc(C(=O)N(C)C)n1)C2] | LC-MS calc. for C₃₇H₃₈F₃N₈O₅ [M + H]⁺: m/z = 731.3; Found: 731.3. |
| 106 | 6-((8-(4-((5-acetyl-2- (cyclopropanecarboxamido) pyridin-4-yl)amino)-3- methoxypyridin-2-yl)- 1-ethyl-6-fluoro-2- oxo-1,2,3,5-tetrahydro-4H- benzo[e][1,4]diazepin- 4-yl)methyl)-N,N- dimethylpicolinamide | 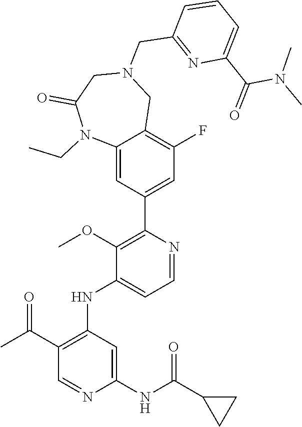 [SMILES: CCN1C(=O)CN(Cc2cccc(C(=O)N(C)C)n2)Cc2c(F)cc(-c3nccc(Nc4cc(NC(=O)C5CC5)ncc4C(C)=O)c3OC)cc21] | LC-MS calc. for C₃₇H₄₀FN₈O₅ [M + H]⁺: m/z = 695.3; Found: 695.4. |
| 107 | 6-((8-(4-((5-acetyl-2- (cyclopropanecarboxamido) pyridin-4-yl)amino)-3- methoxypyridin-2-yl)- 1-methyl-2-oxo-1,2,3,5- tetrahydro-4H- benzo[e][1,4]diazepin- 4-yl)methyl)-N,N- dimethylpicolinamide | 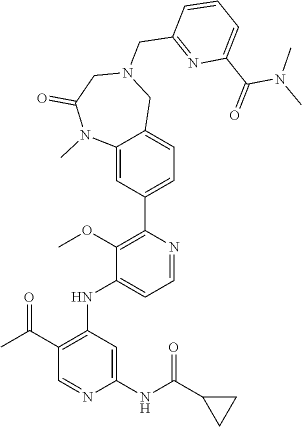 [SMILES: COc1c(Nc2cc(NC(=O)C3CC3)ncc2C(C)=O)ccnc1-c1ccc2c(c1)N(C)C(=O)CN(Cc1cccc(C(=O)N(C)C)n1)C2] | LC-MS calc. for C₃₆H₃₉N₈O₅ [M + H]⁺: m/z = 633.3; Found: 663.4. |
| 108 | 6-((8-(4-((5-acetyl-2- (cyclopropanecarboxamido) pyridin-4-yl)amino)-3- methoxypyridin-2-yl)- 1-isopropyl-2-oxo- 1,2,3,5-tetrahydro-4H- benzo[e][1,4]diazepin- 4-yl)methyl)-N,N- dimethylpicolinamide | 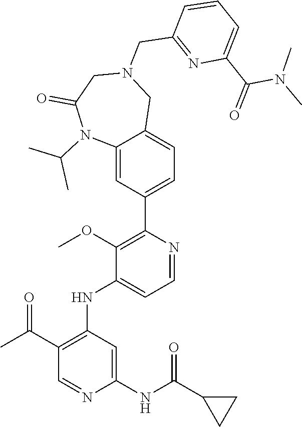 [SMILES: COc1c(Nc2cc(NC(=O)C3CC3)ncc2C(C)=O)ccnc1-c1ccc2c(c1)N(C(C)C)C(=O)CN(Cc1cccc(C(=O)N(C)C)n1)C2] | LC-MS calc. for C₃₈H₄₃N₈O₅ [M + H]⁺: m/z = 691.3; Found: 691.6. |
| 109 | 6-((8-(2-((5-acetyl-2- (cyclopropanecarboxamido) pyridin-4-yl)amino)-3- methoxypyridin-4-yl)- 1-isopropyl-2-oxo- 1,2,3,5-tetrahydro-4H- benzo[e][1,4]diazepin- 4-yl)methyl)-N,N- dimethylpicolinamide | 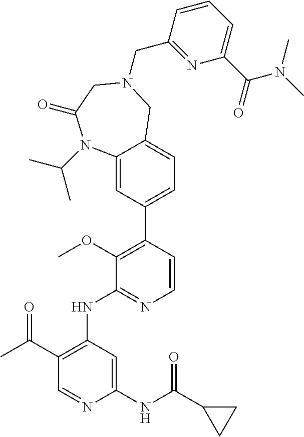 [SMILES: COc1c(-c2ccc3c(c2)N(C(C)C)C(=O)CN(Cc2cccc(C(=O)N(C)C)n2)C3)ccnc1Nc1cc(NC(=O)C2CC2)ncc1C(C)=O] | LC-MS calc. for C₃₈H₄₃N₈O₅ [M + H]⁺: m/z = 691.3; Found: 691.7. |
| 110 | 6-((8-(4-((5-acetyl-2- ((1R,2S)-2- fluorocyclopropane-1- carboxamido)pyridin- 4-yl)amino)-3- methoxypyridin-2-yl)- 1-methyl-2-oxo- 1,2,3,5-tetrahydro-4H- benzo[e][1,4]diazepin- 4-yl)methyl)-N,N- dimethylpicolinamide | 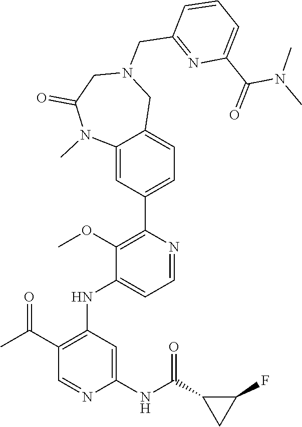 [SMILES: COc1c(Nc2cc(NC(=O)[C@H]3C[C@@H]3F)ncc2C(C)=O)ccnc1-c1ccc2c(c1)N(C)C(=O)CN(Cc1cccc(C(=O)N(C)C)n1)C2] | LC-MS calc. for C₃₆H₃₈FN₈O₅ [M + H]⁺: m/z = 681.3; Found: 681.5. |
| 111 | 6-((8-(3-((5-acetyl-2- ((1S,2S)-2- methylcyclopropane-1- carboxamido)pyridin- 4-yl)amino)-5-fluoro- 2-methoxyphenyl)-1- (2,2-difluoroethyl)-2-oxo- 1,2,3,5-tetrahydro-4H- pyrido[3,2-e][1,4] diazepin-4-yl)methyl)- N,N- dimethylpicolinamide | 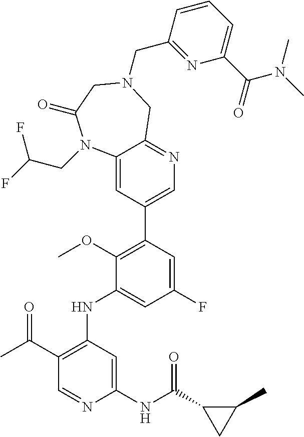 [SMILES: COc1c(Nc2cc(NC(=O)[C@H]3C[C@@H]3C)ncc2C(C)=O)cc(F)cc1-c1cnc2c(c1)N(CC(F)F)C(=O)CN(Cc1cccc(C(=O)N(C)C)n1)C2] | LC-MS calc. for C₃₈H₄₀F₃N₈O₅ [M + H]⁺: m/z = 745.3; Found: 745.6. |
| 112 | 6-((8-(3-((5-acetyl-2- ((1R,2R)-2- methylcyclopropane-1- carboxamido)pyridin- 4-yl)amino)-5-fluoro- 2-methoxyphenyl)-1- (2,2-difluoroethyl)-2- oxo-1,2,3,5- tetrahydro-4H- pyrido[3,2-e][1,4] diazepin-4- yl)methyl)-N,N- dimethylpicolinamide | 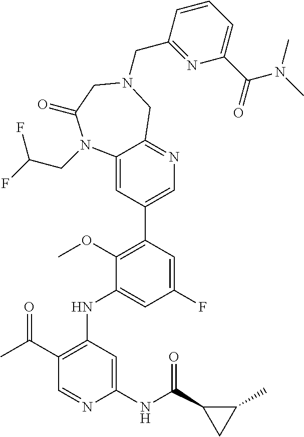 [SMILES: COc1c(Nc2cc(NC(=O)[C@@H]3C[C@H]3C)ncc2C(C)=O)cc(F)cc1-c1cnc2c(c1)N(CC(F)F)C(=O)CN(Cc1cccc(C(=O)N(C)C)n1)C2] | LC-MS calc. for C₃₈H₄₀F₃N₈O₅ [M + H]⁺: m/z = 745.3; Found: 745.6. |
| 113 | 6-(8-(3-((5-acetyl-2- (cyclopropanecarboxamido) pyridin-4- yl)amino)-2- methoxyphenyl)-1- (2,2-difluoroethyl)-2- oxo-2,3,4,5- tetrahydro-1H- pyrido[3,2-e][1,4] diazepine-4- carbonyl)-N,N- dimethylpicolinamide | 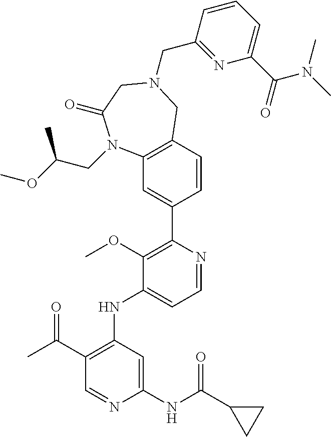 | LC-MS calc. for C₃₉H₄₅N₈O₆ [M + H]⁺: m/z = 721.3; Found: 721.4. |
| 114 | 6-((8-(4-((5-acetyl-2- ((1S,2R)-2- fluorocyclopropane-1- carboxamido)pyridin- 4-yl)amino)-3- methoxypyridin-2-yl)- 2-oxo-1-((S)- tetrahydrofuran-3-yl)- 1,2,3,5-tetrahydro-4H- benzo[e][1,4]diazepin- 4-yl)methyl)-N,N- dimethylpicolinamide | 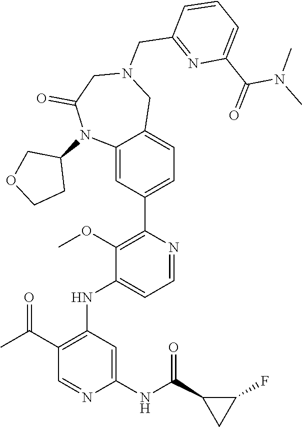 [SMILES: COc1c(Nc2cc(NC(=O)[C@@H]3C[C@H]3F)ncc2C(C)=O)ccnc1-c1ccc2c(c1)N([C@H]1CCOC1)C(=O)CN(Cc1cccc(C(=O)N(C)C)n1)C2] | LC-MS calc. for C₃₉H₄₂FN₈O₆ [M + H]⁺: m/z = 737.3; Found: 737.3. ¹H NMR (400 MHz, DMSO-d₆) δ 11.58 (s, 1H), 11.37 (s, 1H), 9.00 (s, 1H), 8.47 (d, J = 5.5 Hz, 1H), 8.35 (s, 1H), 8.03 (t, J = 7.8 Hz, 1H), 7.89 (d, J = 7.1 Hz, 2H), 7.64 (t, J = 7.7 Hz, 3H), 7.57 (d, J = 7.7 Hz, 1H), 5.01 (d, J = 3.3 Hz, 1H), 4.84 (d, J = 3.7 Hz, 1H), 4.41 (s, 1H), 4.26 (s, 2H), 3.98 (dd, J = 13.6, 8.2 Hz, 3H), 3.69 (d, J = 7.4 Hz, 2H), 3.56 (s, 3H), 3.46 (s, 2H), 3.03 (d, J = 8.4 Hz, 3H), 2.96 (d, J = 11.1 Hz, 3H), 2.69 (s, 3H), 2.67-2.57 (m, 1H), 2.29 (d, J = 31.2 Hz, 2H), 1.66- 1.52 (m, 1H), 1.28 (td, J = 13.0, 6.4 Hz, 1H). |
| 115 | 6-((8-(2-((5-acetyl-2- (cyclopropanecarboxamido) pyridin-4- yl)amino)-3-methoxy- pyridin-4-yl)-1- isopropyl-2-oxo-1,2,3,5- tetrahydro-4H-pyrido [3,2-e][1,4]diazepin-4- yl)methyl)-N,N- dimethylpicolinamide | 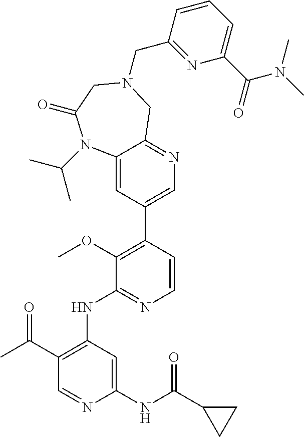 [SMILES: COc1c(-c2cnc3c(c2)N(C(C)C)C(=O)CN(Cc2cccc(C(=O)N(C)C)n2)C3)ccnc1Nc1cc(NC(=O)C2CC2)ncc1C(C)=O] | LC-MS calc. for C₃₇H₄₂N₉O₅ [M + H]⁺: m/z = 692.3; Found: 692.2. |
| 116 | 6-((8-(2-((5-acetyl-2- (cyclopropanecarboxamido) pyridin-4-yl)amino)-3- methoxypyridin-4-yl)- 1-(2,2-difluoroethyl)- 2-oxo-1,2,3,5- tetrahydro-4H- pyrido[3,2-e][1,4] diazepin-4-yl)methyl)- N,N-dimethylpicolinamide | 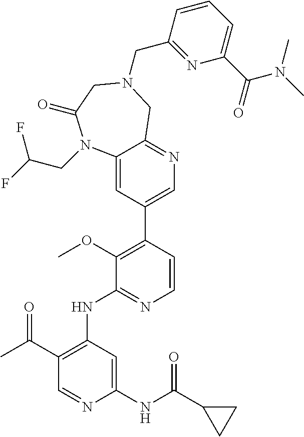 [SMILES: COc1c(-c2cnc3c(c2)N(CC(F)F)C(=O)CN(Cc2cccc(C(=O)N(C)C)n2)C3)ccnc1Nc1cc(NC(=O)C2CC2)ncc1C(C)=O] | LC-MS calc. for C₃₆H₃₈F₂N₉O₅ [M + H]⁺: m/z = 714.3; Found: 714.1. |
| 117 | 6-((8-(4-((5-acetyl-2- (cyclopropanecarboxamido) pyridin-4- yl)amino)-3- methoxypyridin-2-yl)- 1-neopentyl-2-oxo- 1,2,3,5- tetrahydro-4H- pyrido[3,2-e][1,4] diazepin-4- yl)methyl)-N,N- dimethylpicolinamide | 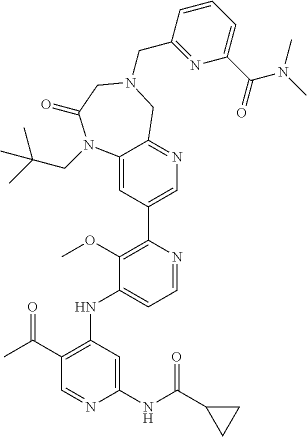 [SMILES: COc1c(Nc2cc(NC(=O)C3CC3)ncc2C(C)=O)ccnc1-c1cnc2c(c1)N(CC(C)(C)C)C(=O)CN(Cc1cccc(C(=O)N(C)C)n1)C2] | LC-MS calc. for C₃₉H₄₆N₉O₅ [M + H]⁺: m/z = 720.4; Found: 720.4. |
| 118 | (S)-6-((8-(4-((5-acetyl-2- (cyclopropanecarboxamido) pyridin-4-yl)amino)-3- methoxypyridin-2-yl)- 2-oxo-1- (tetrahydrofuran-3-yl)- 1,2,3,5-tetrahydro-4H- pyrido[3,2-e][1,4] diazepin-4- yl)methyl)-N,N- dimethylpicolinamide | 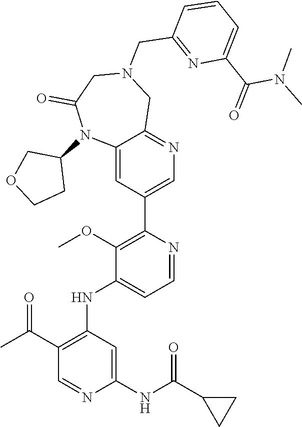 [SMILES: COc1c(Nc2cc(NC(=O)C3CC3)ncc2C(C)=O)ccnc1-c1cnc2c(c1)N([C@H]1CCOC1)C(=O)CN(Cc1cccc(C(=O)N(C)C)n1)C2] | LC-MS calc. for C₃₈H₄₂N₉O₆ [M + H]⁺: m/z = 720.3; Found: 720.1. |
| 119 | 6-((8-(4-((5-acetyl-2- ((1S,2R)-2- fluorocyclopropane-1- carboxamido)pyridin- 4-yl)amino)-3- methoxypyridin-2-yl)- 1-cyclobutyl-2-oxo- 1,2,3,5-tetrahydro-4H- pyrido[3,2-e][1,4] diazepin-4- yl)methyl)-N,N- dimethylpicolinamide | 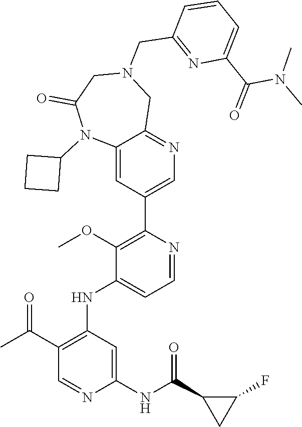 [SMILES: COc1c(Nc2cc(NC(=O)[C@@H]3C[C@H]3F)ncc2C(C)=O)ccnc1-c1cnc2c(c1)N(C1CCC1)C(=O)CN(Cc1cccc(C(=O)N(C)C)n1)C2] | LC-MS calc. for C₃₈H₄₁FN₉O₅ [M + H]⁺: m/z = 722.3; Found: 722.1. |
| 120 | 6-((8-(4-((5-acetyl-2- (cyclopropanecarboxamido) pyridin-4-yl)amino)-3- methoxypyridin-2-yl)- 1-(2,2-difluoroethyl)- 2-oxo-1,2,3,5- tetrahydro-4H- benzo[e][1,4]diazepin- 4-yl)methyl)-N,N- dimethylpicolinamide | 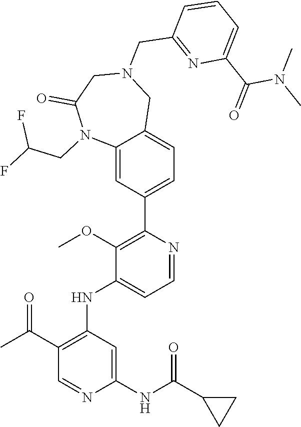 [SMILES: COc1c(Nc2cc(NC(=O)C3CC3)ncc2C(C)=O)ccnc1-c1ccc2c(c1)N(CC(F)F)C(=O)CN(Cc1cccc(C(=O)N(C)C)n1)C2] | LC-MS calc. for C₃₇H₃₉F₂N₈O₅ [M + H]⁺: m/z = 713.3; Found: 713.2. ¹H NMR (400 MHz, CDCl₃) δ 12.59 (s, 1H), 12.21 (s, 1H), 8.78-8.69 (m, 3H), 8.00-7.89 (m, 3H), 7.73 (d, J = 5.7 Hz, 1H), 7.69-7.58 (m, 3H), 6.17 (t, J = 55.5 Hz, 1H), 4.51 (s, 2H), 4.40 (d, J = 30.7 Hz, 4H), 3.86 (s, 2H), 3.64 (s, 3H), 3.15 (s, 3H), 3.06 (s, 3H), 2.72 (s, 3H), 1.98 (td, J = 7.5, 3.9 Hz, 1H), 1.29-1.02 (m, 4H). |
| 121 | (S)-6-((8-(4-((5-acetyl- 2- (cyclopropanecarboxamido) pyridin-4- yl)amino)-3- methoxypyridin-2-yl)- 2-oxo-1- (tetrahydrofuran-3-yl)- 1,2,3,5-tetrahydro-4H- benzo[e][1,4]diazepin- 4-yl)methyl)-N,N- dimethylpicolinamide | 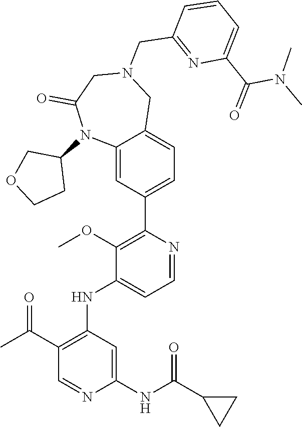 [SMILES: COc1c(Nc2cc(NC(=O)C3CC3)ncc2C(C)=O)ccnc1-c1ccc2c(c1)N([C@H]1CCOC1)C(=O)CN(Cc1cccc(C(=O)N(C)C)n1)C2] | LC-MS calc. for C₃₉H₄₃N₈O₆ [M + H]⁺: m/z = 719.3; Found: 719.2 ¹H NMR (600 MHz, DMSO-d₆) δ 11.60 (s, 1H), 11.26 (s, 1H), 8.99 (s, 1H), 8.49 (s, 1H), 8.42 (s, 1H), 8.03 (t, J = 7.7 Hz, 1H), 7.89 (d, J = 8.3 Hz, 2H), 7.70- 7.60 (m, 3H), 7.57 (d, J = 7.7 Hz, 1H), 5.06-4.94 (m, 1H), 4.47-4.35 (m, 2H), 4.31-4.17 (m, 2H), 3.98 (dd, J = 13.7, 8.0 Hz, 2H), 3.70 (s, 2H), 3.56 (s, 3H), 3.46 (s, 2H), 3.02 (s, 3H), 2.94 (s, 3H), 2.69 (s, 3H), 2.34-2.14 (m, 2H), 2.11- 2.05 (m, 1H), 0.93-0.80 (m, 4H). |
| 122 | 6-((8-(4-((5-acetyl-2- (cyclopropanecarboxamido) pyridin-4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 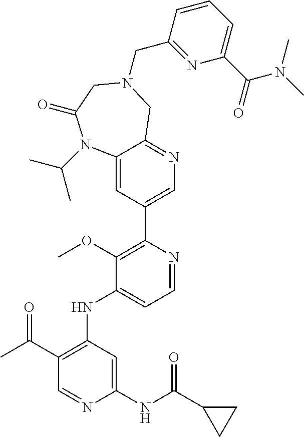 [SMILES: COc1c(Nc2cc(NC(=O)C3CC3)ncc2C(C)=O)ccnc1-c1cnc2c(c1)N(C(C)C)C(=O)CN(Cc1cccc(C(=O)N(C)C)n1)C2] | LC-MS calc. for C₃₇H₄₂N₉O₅ [M + H]⁺: m/z = 692.3; Found: 692.1. ¹H NMR (400 MHz, CDCl₃) δ 12.88 (s, 1H), 12.09 (s, 1H), 9.19 (s, 1H), 8.73-8.63 (m, 3H), 8.30 (s, 1H), 7.90 (t, J = 7.7 Hz, 1H), 7.69-7.59 (m, 3H), 4.80 (p, J = 7.0 Hz, 1H), 4.41 (d, J = 15.1 Hz, 4H), 3.69 (s, 5H), 3.10 (d, J = 28.9 Hz, 6H), 2.71 (s, 3H), 2.01 (td, J = 7.7, 4.0 Hz, 1H), 1.44 (s, 6H), 1.14 (p, J = 4.2 Hz, 2H), 1.05 (dq, J = 7.8, 3.8 Hz, 2H). |
| 123 | 6-((8-(4-((5-acetyl-2- ((1S,2S)-2- methylcyclopropane-1- carboxamido)pyridin- 4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 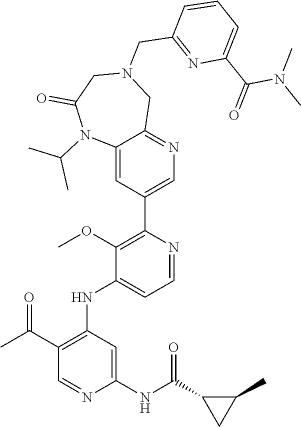 [SMILES: COc1c(Nc2cc(NC(=O)[C@H]3C[C@@H]3C)ncc2C(C)=O)ccnc1-c1cnc2c(c1)N(C(C)C)C(=O)CN(Cc1cccc(C(=O)N(C)C)n1)C2] | LC-MS calc. for C₃₈H₄₄N₉O₅ [M + H]⁺: m/z = 706.3; Found: 706.3. ¹H NMR (400 MHz, CDCl₃) δ 12.55 (s, 1H), 11.98 (s, 1H), 9.15 (d, J = 1.9 Hz, 1H), 8.68- 8.60 (m, 3H), 8.25 (d, J = 1.9 Hz, 1H), 7.84 (t, J = 7.8 Hz, 1H), 7.65-7.55 (m, 3H), 4.83 (p, J = 6.9 Hz, 1H), 4.19 (s, 2H), 4.11 (s, 2H), 3.67 (s, 3H), 3.40 (s, 2H), 3.13 (s, 3H), 3.09 (s, 3H), 2.70 (s, 4H), 1.71 (dt, J = 8.1, 4.2 Hz, 1H), 1.58-1.48 (m, 1H), 1.42 (s, 6H), 1.31 (dt, J = 8.5, 4.1 Hz, 1H), 1.26 (s, 1H), 1.20 (d, J = 6.0 Hz, 3H), 0.90 (td, J = 7.4, 4.0 Hz, 1H). |
| 124 | 6-((8-(4-((5-acetyl-2- ((1S,2R)-2- methylcyclopropane-1- carboxamido)pyridin- 4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 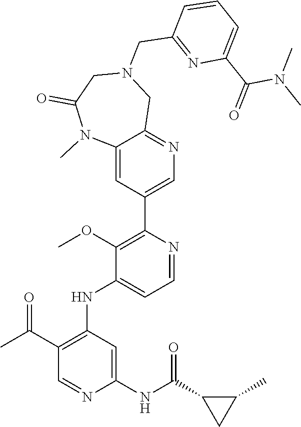 | LC-MS calc. for C₃₈H₄₄N₉O₅ [M + H]⁺: m/z = 706.3; Found: 706.3. |
| 125 | 6-((8-(4-((5-acetyl-2- ((1R,2R)-2- methylcyclopropane- 1- carboxamido)pyridin- 4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 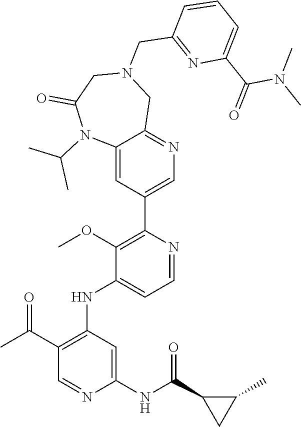 [SMILES: COc1c(Nc2cc(NC(=O)[C@@H]3C[C@H]3C)ncc2C(C)=O)ccnc1-c1cnc2c(c1)N(C(C)C)C(=O)CN(Cc1cccc(C(=O)N(C)C)n1)C2] | LC-MS calc. for C₃₈H₄₄N₉O₅ [M + H]⁺: m/z = 706.3; Found: 706.4. ¹H NMR (400 MHz, CDCl₃) δ 12.74 (s, 1H), 12.06 (s, 1H), 9.19 (d, J = 1.8 Hz, 1H), 8.70-8.64 (m, 3H), 8.30 (d, J = 1.8 Hz, 1H), 7.89 (t, J = 7.8 Hz, 1H), 7.66-7.60 (m, 3H), 4.81 (p, J = 6.9 Hz, 1H), 4.35 (d, J = 19.7 Hz, 4H), 3.69 (s, 3H), 3.63 (s, 3H), 3.14 (s, 3H), 3.07 (s, 3H), 2.71 (s, 3H), 1.72 (dt, J = 8.1, 4.2 Hz, 1H), 1.58-1.49 (m, 1H), 1.44 (s, 6H), 1.32 (dt, J = 8.6, 4.2 Hz, 1H), 1.20 (d, J = 6.0 Hz, 3H), 0.91 (td, J = 7.4, 4.0 Hz, 1H). |
| 126 | 6-((8-(4-((5-acetyl-2- ((1R,2S)-2- fluorocyclopropane-1- carboxamido)pyridin- 4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 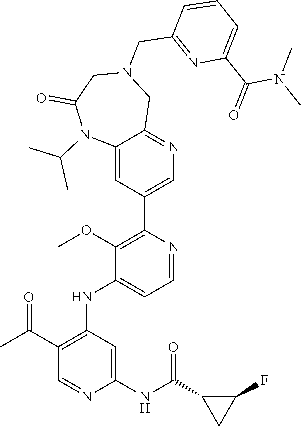 [SMILES: COc1c(Nc2cc(NC(=O)[C@H]3C[C@@H]3F)ncc2C(C)=O)ccnc1-c1cnc2c(c1)N(C(C)C)C(=O)CN(Cc1cccc(C(=O)N(C)C)n1)C2] | LC-MS calc. for C₃₇H₄₁FN₉O₅ [M + H]⁺: m/z = 710.3; Found: 710.2. ¹H NMR (400 MHz, CDCl₃) δ 13.05 (s, 1H), 12.12 (s, 1H), 9.19 (s, 1H), 8.76-8.60 (m, 3H), 8.30 (s, 1H), 7.91 (t, J = 7.8 Hz, 1H), 7.63 (dd, J = 8.5, 5.0 Hz, 3H), 4.79 (d, J = 6.6 Hz, 2H), 4.45 (s, 2H), 4.40 (s, 2H), 3.71 (d, J = 11.4 Hz, 10H), 3.10 (d, J = 30.8 Hz, 7H), 2.72 (s, 3H), 2.51 (dt, J = 16.3, 8.1 Hz, 1H), 1.64 (s, 1H), 1.45 (s, 7H). |
| 127 | 6-((8-(4-((5-acetyl-2- ((1S,2S)-2- fluorocyclopropane-1- carboxamido)pyridin- 4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 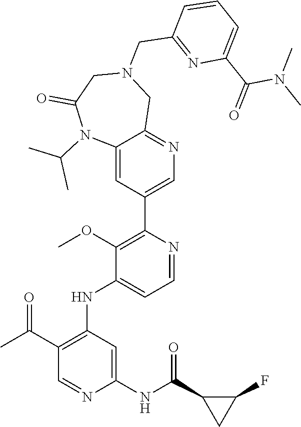 [SMILES: COc1c(Nc2cc(NC(=O)[C@@H]3C[C@@H]3F)ncc2C(C)=O)ccnc1-c1cnc2c(c1)N(C(C)C)C(=O)CN(Cc1cccc(C(=O)N(C)C)n1)C2] | LC-MS calc. for C₃₇H₄₁FN₉O₅ [M + H]⁺: m/z = 710.3; Found: 710.3. |
| 128 | 6-((8-(4-((5-acetyl-2- ((1S,2R)-2- fluorocyclopropane-1- carboxamido)pyridin- 4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 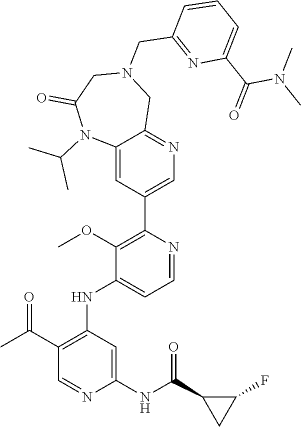 [SMILES: COc1c(Nc2cc(NC(=O)[C@@H]3C[C@H]3F)ncc2C(C)=O)ccnc1-c1cnc2c(c1)N(C(C)C)C(=O)CN(Cc1cccc(C(=O)N(C)C)n1)C2] | LC-MS calc. for C₃₇H₄₁FN₉O₅ [M + H]⁺: m/z = 710.3; Found: 710.2. ¹H NMR (400 MHz, CDCl₃) δ 13.05 (s, 1H), 12.13 (s, 1H), 9.19 (d, J = 2.1 Hz, 1H), 8.77- 8.62 (m, 3H), 8.30 (s, 1H), 7.91 (t, J = 7.7 Hz, 1H), 7.64 (dt, J = 7.6, 4.7 Hz, 3H), 4.45 (d, J = 15.8 Hz, 5H), 3.75 (s, 2H), 3.70 (s, 3H), 3.10 (d, J = 31.9 Hz, 6H), 2.72 (s, 3H), 2.51 (dt, J = 16.6, 8.5 Hz, 1H), 1.73- 1.60 (m, 1H), 1.46 (d, J = 6.8 Hz, 7H). |
| 129 | 6-((8-(4-((5-acetyl-2- ((1R,2R)-2- fluorocyclopropane-1- carboxamido)pyridin- 4-yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 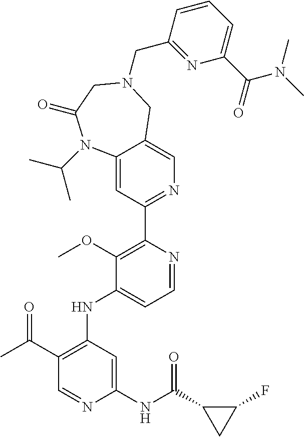 [SMILES: COc1c(Nc2cc(NC(=O)[C@H]3C[C@H]3F)ncc2C(C)=O)ccnc1-c1cc2c(cn1)CN(Cc1cccc(C(=O)N(C)C)n1)CC(=O)N2C(C)C] | LC-MS calc. for C₃₇H₄₁FN₉O₅ [M + H]⁺: m/z = 710.3; Found: 710.3. |
| 130 | 6-((8-(4-((2- (cyclopropane- carboxamido)-5- propionylpyridin-4- yl)amino)-3- methoxypyridin-2-yl)- 1-isopropyl-2-oxo- 1,2,3,5-tetrahydro-4H- pyrido[3,2-e][1,4] diazepin-4- yl)methyl)-N,N- dimethylpicolinamide | 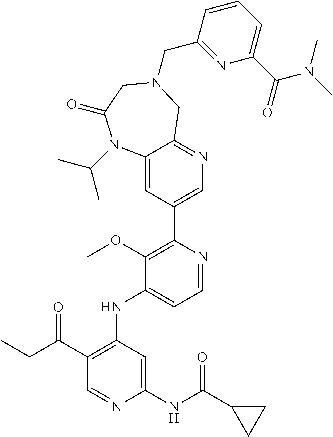 [SMILES: CCC(=O)c1cnc(NC(=O)C2CC2)cc1Nc1ccnc(-c2cnc3c(c2)N(C(C)C)C(=O)CN(Cc2cccc(C(=O)N(C)C)n2)C3)c1OC] | LC-MS calc. for C₃₈H₄₄N₉O₅ [M + H]⁺: m/z = 706.3; Found: 706.3. |
| 131 | 6-((8-(4-((5-acetyl-2- (cyclopropanecarboxamido) pyridin-4-yl)amino)-3- methoxypyridin-2-yl)- 1-(2,2-difluoroethyl)- 2-oxo- 1,2,3,5-tetrahydro-4H- pyrido[3,2-e][1,4] diazepin-4- yl)methyl)-N,N- dimethylpicolinamide | 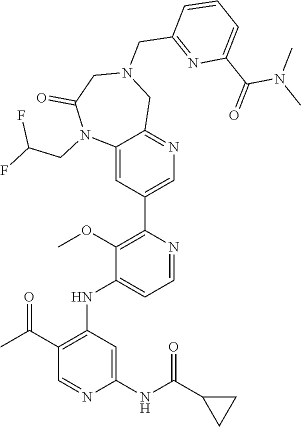 [SMILES: COc1c(Nc2cc(NC(=O)C3CC3)ncc2C(C)=O)ccnc1-c1cnc2c(c1)N(CC(F)F)C(=O)CN(Cc1cccc(C(=O)N(C)C)n1)C2] | LC-MS calc. for C₃₆H₃₈F₂N₉O₅ [M + H]⁺: m/z = 714.3; Found: 714.2. |
| 132 | 6-((8-(4-((5-acetyl-2- ((1S,2R)-2- fluorocyclopropane-1- carboxamido)pyridin- 4-yl)amino)-3- methoxypyridin-2-yl)- 1-(2,2-difluoroethyl)- 2-oxo- 1,2,3,5-tetrahydro-4H- pyrido[3,2-e][1,4] diazepin-4- yl)methyl)-N,N- dimethylpicolinamide | 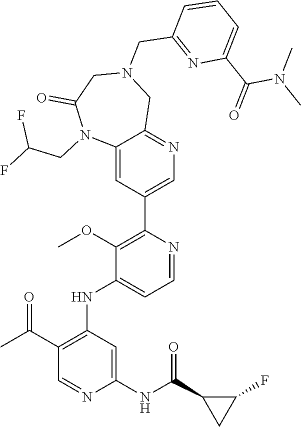 [SMILES: COc1c(Nc2cc(NC(=O)[C@@H]3C[C@H]3F)ncc2C(C)=O)ccnc1-c1cnc2c(c1)N(CC(F)F)C(=O)CN(Cc1cccc(C(=O)N(C)C)n1)C2] | LC-MS calc. for C₃₆H₃₇F₃N₉O₅ [M + H]⁺: m/z = 732.3; Found: 732.2. |
| 133 | 6-((8-(3-((5-acetyl-2- (cyclopropanecarboxamido) pyridin-4-yl)amino)-5- chloro-2-methoxyphenyl)- 1-(2,2-difluoroethyl)-2- oxo-1,2,3,5-tetrahydro-4H- pyrido[3,2-e][1,4] diazepin-4- yl)methyl)-N,N- dimethylpicolinamide | 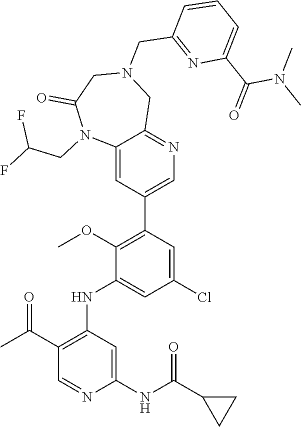 [SMILES: COc1c(Nc2cc(NC(=O)C3CC3)ncc2C(C)=O)cc(Cl)cc1-c1cnc2c(c1)N(CC(F)F)C(=O)CN(Cc1cccc(C(=O)N(C)C)n1)C2] | LC-MS calc. for C₃₇H₃₈ClF₂N₈O₅ [M + H]⁺: m/z = 747.3; Found: 747.3. |
| 134 | (R)-6-((8-(4-((5- acetyl-2- (cyclopropanecarboxamido) pyridin-4-yl)amino)-3- methoxypyridin-2-yl)- 1-(1-methoxypropan- 2-yl)-2-oxo- 1,2,3,5-tetrahydro-4H- pyrido[3,2-e][1,4] diazepin-4- yl)methyl)-N,N- dimethylpicolinamide | 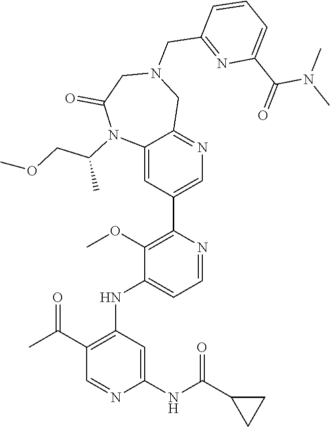 | LC-MS calc. for C₃₈H₄₄N₉O₆ [M + H]⁺: m/z = 722.3; Found: 722.2. |

### Example 135. (1R,2S)—N-(5-acetyl-4-((5-fluoro-2-methoxy-3-(1-methyl-4-((6-(morpholine-4-carbonyl)pyridin-2-yl)methyl)-2-oxo-2,3,4,5-tetrahydro-1H-benzo[e][1,4]diazepin-8-yl)phenyl)amino)pyridin-2-yl)-2-fluorocyclopropane-1-carboxamide

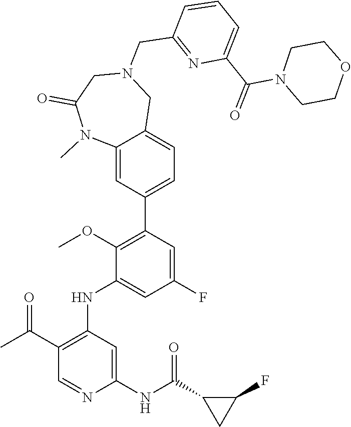 [SMILES: COc1c(Nc2cc(NC(=O)[C@H]3C[C@@H]3F)ncc2C(C)=O)cc(F)cc1-c1ccc2c(c1)N(C)C(=O)CN(Cc1cccc(C(=O)N3CCOCC3)n1)C2]

### Step 1. 6-(Morpholine-4-carbonyl)picolinaldehyde

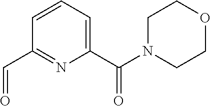 [SMILES: O=Cc1cccc(C(=O)N2CCOCC2)n1]

1-[Bis(dimethylamino)methylene]-1H-1,2,3-triazolo[4,5-b]pyridinium 3-oxid hexafluorophosphate (755 mg, 1.99 mmol) was added to a stirring solution of methyl 6-formylpicolinate (200 mg, 1.32 mmol), morpholine (139 μL, 1.59 mmol) and N,N-diisopropylethylamine (692 μL, 3.97 mmol) in dimethylacetamide (3 mL) at rt. The reaction mixture was stirred for 3 hours. The product mixture was diluted with EtOAc (40 mL) and washed with sat. brine (aq) solution (3×40 mL). The organic layer was dried over Na₂SO₄, filtered, and concentrated under reduced pressure. The residue obtained was purified by silica gel column chromatography (0-10% MeOH/DCM) to yield the title compound (260 mg, 89% yield). LCMS calc. for C₁₁H₁₃N₂O₃ [M+H]⁺: m/z=221.1; Found: 221.3.

### Step 2. 8-Bromo-4-((6-(morpholine-4-carbonyl)pyridin-2-yl)methyl)-1,3,4,5-tetrahydro-2H-benzo[e][1,4]diazepin-2-one

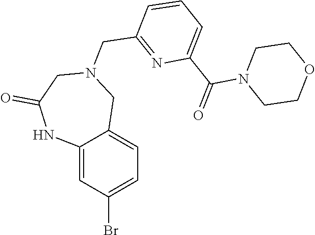 [SMILES: O=C1CN(Cc2cccc(C(=O)N3CCOCC3)n2)Cc2ccc(Br)cc2N1]

Sodium triacetoxyborohydride (319 mg, 1.5 mmol) was added to a stirring solution of 8-bromo-1,3,4,5-tetrahydro-2H-benzo[e][1,4]diazepin-2-one (100 mg, 0.42 mmol), 6-(morpholine-4-carbonyl)picolinaldehyde (133 mg, 0.603 mmol) and acetic acid (52 μL, 0.90 mmol) in DMSO (3 mL). The reaction mixture was stirred for 16 hours at rt. The product mixture was diluted with water (30 mL) and extracted with EtOAc (3×20 mL). The combined organic layers were dried over Na₂SO₄, filtered, and concentrated under reduced pressure to yield the title compound (104 mg, 78% yield) which was used without further purification. LCMS calc. for C₂₀H₂₂BrN₄O₃[M+H]⁺: m/z=445.1/447.1; Found: 445.2/447.2.

### Step 3. (1R,2S)—N-(5-Acetyl-4-((5-fluoro-2-methoxy-3-(1-methyl-4-((6-(morpholine-4-carbonyl)pyridin-2-yl)methyl)-2-oxo-2,3,4,5-tetrahydro-1H-benzo[e][1,4]diazepin-8-yl)phenyl)-amino)pyridin-2-yl)-2-fluorocyclopropane-1-carboxamide

The title compound was prepared in accordance with the synthetic protocols set forth in Example 71 Step 1 and Example 36 Step 2 using appropriate intermediates, as well as commercial starting materials. LCMS calc. for C₃₉H₄₀F₂N₇O₆[M+H]⁺: m/z=740.3; Found: 740.2.

### Examples 136-137

Examples 136-137 are shown below in Table 15 and were prepared in accordance with the synthetic protocols set forth in Example 135 using the appropriate intermediates, as well as commercial starting materials. The following compounds are TFA salts unless otherwise noted.

TABLE 15
Examples 136-137

| | | | Analytical Data (LCMS and/or |
|---|---|---|---|
| Ex. | Name | Structure | NMR) |
| 136 | N-(5-acethyl-4- ((3-(4-((6-(3,3- difluoropyrrolidin- 1- carbonyl)pyridin- 2-yl)methyl)- 1-ethyl-2-oxo- 2,3,4,5- tetrahydro-1H- benzo[e][1,4] diazepin-8-yl)-2- methoxyphenyl) amino)pyridin- 2- yl)cyclopropane- carboxamide | 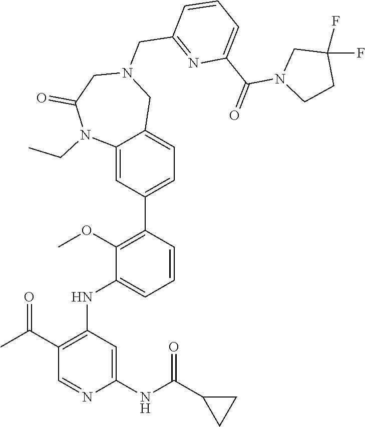 [SMILES: CCN1C(=O)CN(Cc2cccc(C(=O)N3CCC(F)(F)C3)n2)Cc2ccc(-c3cccc(Nc4cc(NC(=O)C5CC5)ncc4C(C)=O)c3OC)cc21] | LC-MS calc. for C₄₀H₄₂F₂N₇O₅ [M + H]⁺: m/z = 738.3; Found: 738.7. ¹H NMR (400 MHz, DMSO-d₆) δ 11.05 (s, 1H), 11.02 (d, J = 8.5 Hz, 1H), 8.85 (s, 1H), 8.06 (t, J = 7.7 Hz, 1H), 7.97 (s, 1H), 7.82 (dd, J = 22.7, 7.7 Hz, 1H), 7.73 (dd, J = 9.6, 5.6 Hz, 2H), 7.58-7.48 (m, 3H), 7.36-7.27 (m, 2H), 4.33- 4.26 (m, 2H), 4.02-3.91 (m, 6H), 3.80-3.75 (m, 2H), 3.46 (s, 2H), 3.36 (s, 3H), 2.65 (s, 3H), 2.44 (dd, J = 14.5, 7.7 Hz, 2H), 2.01 (s, 1H), 1.12 (t, J = 7.0 Hz, 3H), 0.85-0.76 (m, 4H). |
| 137 | N-(5-acetyl-4- ((3-(1-ethyl-4- ((1-methyl-1H- pyrazol-4- yl)methyl)-2- oxo-2,3,4,5- tetrahydro-1H- benzo[e][1,4] diazepin-8-yl)-2- methoxyphenyl) amino)pyridin- 2-yl)cyclo- propanecarboxamide | 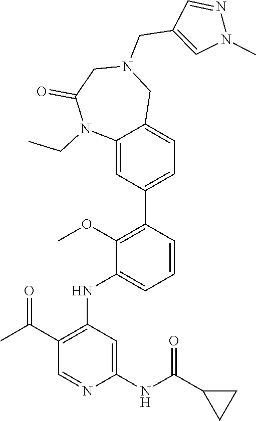 [SMILES: CCN1C(=O)CN(Cc2cnn(C)c2)Cc2ccc(-c3cccc(Nc4cc(NC(=O)C5CC5)ncc4C(C)=O)c3OC)cc21] | LC-MS calc. for C₃₄H₃₈N₇O₄ [M + H]⁺: m/z = 608.3; Found: 608.6. ¹H NMR (400 MHz, DMSO-d₆) δ 11.02 (s, 1H), 10.97 (s, 1H), 8.82 (s, 1H), 7.98 (s, 1H), 7.90 (s, 1H), 7.72 (s, 1H), 7.62 (d, J = 8.0 Hz, 1H), 7.58 (s, 1H), 7.55 (d, J = 7.8 Hz, 1H), 7.49 (dd, J = 7.4, 1.8 Hz, 1H), 7.31 (d, J = 7.6 Hz, 1H), 7.28-7.26 (m, 1H), 4.24 (s, 3H), 3.98 (s, 3H), 3.85 (s, 2H), 3.34 (s, 2H), 2.96 (s, 3H), 2.62 (s, 2H), 2.51 (s, 2H), 1.99-1.95 (m, 1H), 1.11 (t, J = 7.0 Hz, 3H), 0.81-0.73 (m, 4H). |

### Example 138: N-(5-acetyl-4-((3-(1-ethyl-4-((6-((3aR,6aR)-hexahydro-1H-furo[3,4-c]pyrrole-5-carbonyl)pyridin-2-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide

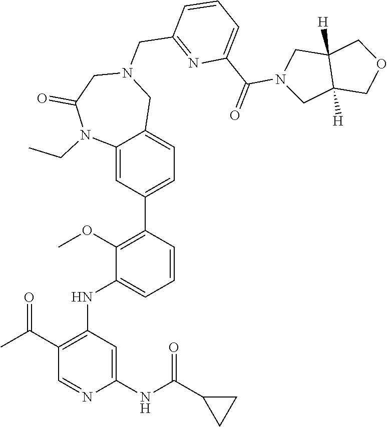 [SMILES: CCN1C(=O)CN(Cc2cccc(C(=O)N3C[C@@H]4COC[C@H]4C3)n2)Cc2ccc(-c3cccc(Nc4cc(NC(=O)C5CC5)ncc4C(C)=O)c3OC)cc21]

### Step 1. N-(5-acetyl-4-((3-(I-ethyl-2-oxo-2,3,4,5-tetrahydro-1H-benzo[e][1,4]diazepin-8-yl)-2-methoxyphenyl)amino)pyridin-2-yl)cyclopropanecarboxamide

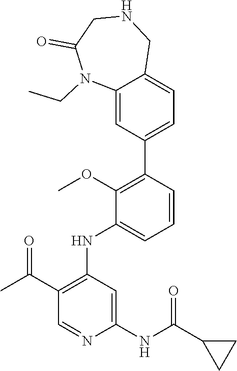 [SMILES: CCN1C(=O)CNCc2ccc(-c3cccc(Nc4cc(NC(=O)C5CC5)ncc4C(C)=O)c3OC)cc21]

The title compound was prepared as an HCl salt in accordance with the synthetic protocols set forth in Example 64 Steps 1-3 using the appropriate intermediates and commercially available materials. LC-MS calc. for C₂₉H₃₂N₅O₄ [M+H]⁺: m/z=514.2; Found: 514.1. ¹H NMR (400 MHz, DMSO-d₆) δ 11.04 (d, J=14.2 Hz, 2H), 8.85 (s, 1H), 8.04 (t, J=7.8 Hz, 1H), 7.98 (s, 1H), 7.71 (s, 1H), 7.66 (d, J=7.8 Hz, 1H), 7.61 (d, J=7.7 Hz, 1H), 7.55 (s, 2H), 7.51 (dd, J=7.1, 2.5 Hz, 1H), 7.34-7.26 (m, 2H), 3.70-3.64 (m, 16H), 3.56 (t, J=4.7 Hz, 6H), 3.39 (d, J=15.3 Hz, 8H), 2.65 (s, 3H), 1.13 (t, J=7.1 Hz, 3H), 0.81 (dt, J=12.0, 3.6 Hz, 4H).

### Step 2. 6-((3aR,6aR)-hexahydro-1H-furo[3,4-c]pyrrole-5-carbonyl)picolinaldehyde

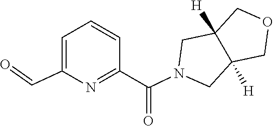 [SMILES: O=Cc1cccc(C(=O)N2C[C@@H]3COC[C@H]3C2)n1]

The title compound was prepared in accordance with the synthetic protocols set forth in Example 135 Step 1 using the appropriate intermediates and commercial materials. LC-MS calc. for C₁₃H₁₅N₂O₃ [M+H]⁺: m/z=247.1; Found: 247.0.

### Step 3. N-(5-acetyl-4-((3-(I-ethyl-4-((6-((3aR,6aR)-hexahydro-1H-furo[3,4-c]pyrrole-5-carbonyl)pyridin-2-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide

A solution of N-(5-acetyl-4-((3-(1-ethyl-2-oxo-2,3,4,5-tetrahydro-1H-benzo[e][1,4]-diazepin-8-yl)-2-methoxyphenyl)amino)pyridin-2-yl)cyclopropanecarboxamide HCl (7.5 mg, 0.014 mmol), 6-((3aR,6aR)-hexahydro-1H-furo[3,4-c]pyrrole-5-carbonyl)picolinaldehyde (10 mg, 0.041 mmol) and acetic acid (10 μL, 0.175 mmol) in DMF (0.3 mL) is stirred at 32° C. for 30 minutes before adding STAB (14 mg, 0.068 mmol). The reaction was stirred at rt for 3 hours, then diluted in MeOH and purified on prep-LCMS (CSH-C18, 17.4-37.4% MeCN/water with 0.2% TFA over 5 min) to afford the title compound (7.2 mg, 620 yield) as a TFA salt. LC-MS calc. for C₄₂H₄₆N₇O₆ [M+H]⁺: m/z=744.4; Found: 744.2.

### Examples 139-159

Examples 139-159 are set forth in Table 16 and were prepared in accordance with the synthetic protocols set forth in Example 138 Step 3 using the appropriate intermediates, as well as commercial starting materials. The following compounds are TFA salts unless otherwise noted.

TABLE 16
Examples 139-159

| | | | Analytical |
|---|---|---|---|
| | | | Data (LCMS |
| Ex. | Name | Structure | and/or NMR) |
| 139 | N-(5-acetyl-4-((3-(1-ethyl- 4-((6-(morpholine-4- carbonyl)pyridin-2- yl)methyl)-2-oxo- 2,3,4,5-tetrahydro-1H- benzo[e][1,4]diazepin-8- yl)-2-methoxyphenyl)- amino)pyridin-2- yl)cyclopropanecarboxamide | 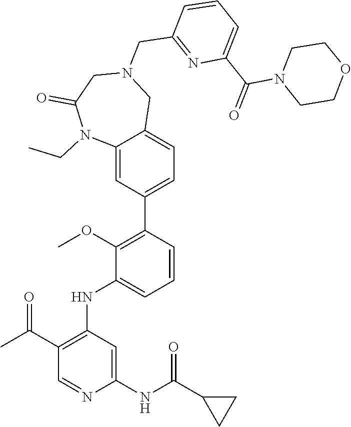 [SMILES: CCN1C(=O)CN(Cc2cccc(C(=O)N3CCOCC3)n2)Cc2ccc(-c3cccc(Nc4cc(NC(=O)C5CC5)ncc4C(C)=O)c3OC)cc21] | LC-MS calc. for C₄₀H₄₄N₇O₆ [M + H]⁺: m/z = 718.3; Found: 718.5. |
| 140 | N-(4-((3-(4- ([1,2,4]triazolo[1,5- a]pyridin-5-ylmethyl)-1- ethyl-2-oxo-2,3,4,5- tetrahydro-1H- benzo[e][1,4]diazepin-8- yl)-2-methoxyphenyl)- amino)-5-acetylpyridin-2- yl)cyclopropanecarboxamide | 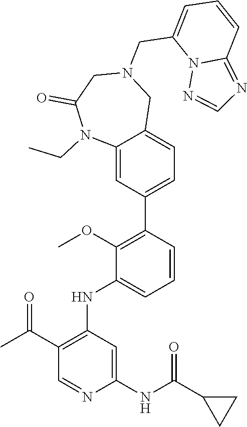 [SMILES: CCN1C(=O)CN(Cc2cccc3ncnn23)Cc2ccc(-c3cccc(Nc4cc(NC(=O)C5CC5)ncc4C(C)=O)c3OC)cc21] | LC-MS calc. for C₃₆H₃₇N₈O₄ [M + H]⁺: m/z = 645.3; Found: 645.2. |
| 141 | N-(4-((3-(4- ([1,2,4]triazolo[1,5- a]pyridin-8-ylmethyl)-1- ethyl-2-oxo-2,3,4,5- tetrahydro-1H- benzo[e][1,4]diazepin-8- yl)-2-methoxyphenyl)- amino)-5-acetylpyridin-2- yl)cyclopropanecarboxamide | 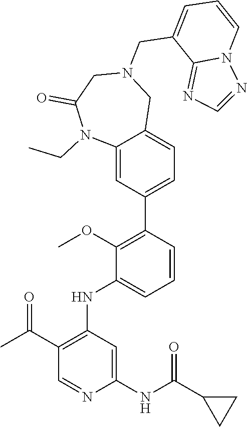 [SMILES: CCN1C(=O)CN(Cc2cccn3ncnc23)Cc2ccc(-c3cccc(Nc4cc(NC(=O)C5CC5)ncc4C(C)=O)c3OC)cc21] | LC-MS calc. for C₃₆H₃₇N₈O₄ [M + H]⁺: m/z = 645.3; Found: 645.2 |
| 142 | N-(5-acetyl-4-((3-(1-ethyl- 4-(imidazo[1,5-a]pyridin- 8-ylmethyl)-2-oxo-2,3,4,5- tetrahydro-1H- benzo[e][1,4]diazepin-8- yl)-2-methoxyphenyl)- amino)pyridin-2- yl)cyclopropanecarboxamide | 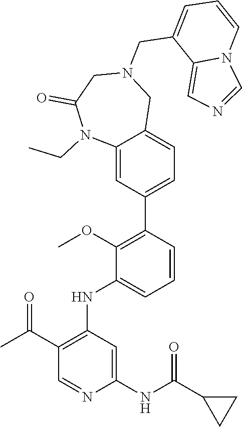 [SMILES: CCN1C(=O)CN(Cc2cccn3cncc23)Cc2ccc(-c3cccc(Nc4cc(NC(=O)C5CC5)ncc4C(C)=O)c3OC)cc21] | LC-MS calc. for C₃₇H₃₈N₇O₄ [M + H]⁺: m/z = 644.3; Found: 644.2. |
| 143 | N-(5-acetyl-4-((3-(1-ethyl- 4-(imidazo[1,2-a]pyridin- 8-ylmethyl)-2-oxo-2,3,4,5- tetrahydro-1H- benzo[e][1,4]diazepin-8- yl)-2-methoxyphenyl)- amino)pyridin-2- yl)cyclopropanecarboxamide | 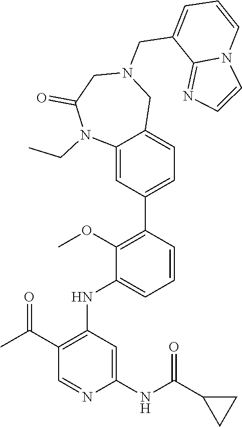 [SMILES: CCN1C(=O)CN(Cc2cccn3ccnc23)Cc2ccc(-c3cccc(Nc4cc(NC(=O)C5CC5)ncc4C(C)=O)c3OC)cc21] | LC-MS calc. for C₃₇H₃₈N₇O₄ [M + H]⁺: m/z = 644.3; Found: 644.2. |
| 144 | N-(5-acetyl-4-((3-(1-ethyl- 2-oxo-4-(pyrazolo[1,5- a]pyridin-7-ylmethyl)- 2,3,4,5-tetrahydro-1H- benzo[e][1,4]diazepin-8- yl)-2-methoxyphenyl)- amino)pyridin-2- yl)cyclopropanecarboxamide | 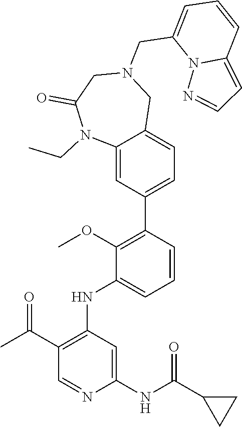 [SMILES: CCN1C(=O)CN(Cc2cccc3ccnn23)Cc2ccc(-c3cccc(Nc4cc(NC(=O)C5CC5)ncc4C(C)=O)c3OC)cc21] | LC-MS calc. for C₃₇H₃₈N₇O₄ [M + H]⁺: m/z = 644.3; Found: 644.2. |
| 145 | N-(5-acetyl-4-((3-(1-ethyl- 4-(imidazo[1,2-a]pyridin- 5-ylmethyl)-2-oxo-2,3,4,5- tetrahydro-1H- benzo[e][1,4]diazepin-8- yl)-2-methoxyphenyl)- amino)pyridin-2- yl)cyclopropanecarboxamide | 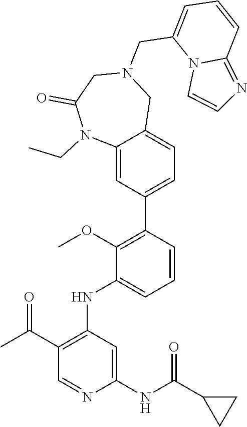 [SMILES: CCN1C(=O)CN(Cc2cccc3nccn23)Cc2ccc(-c3cccc(Nc4cc(NC(=O)C5CC5)ncc4C(C)=O)c3OC)cc21] | LC-MS calc. for C₃₇H₃₈N₇O₄ [M + H]⁺: m/z = 644.3; Found: 644.2 |
| 146 | N-(5-acetyl-4-((3-(1-ethyl- 4-((6- (methylsulfonyl)pyridin-2- yl)methyl)-2-oxo- 2,3,4,5-tetrahydro-1H- benzo[e][1,4]diazepin-8- yl)-2-methoxyphenyl)- amino)pyridin-2- yl)cyclopropanecarboxamide | 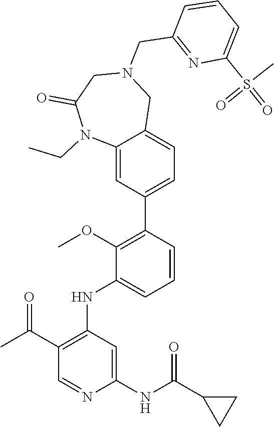 [SMILES: CCN1C(=O)CN(Cc2cccc(S(C)(=O)=O)n2)Cc2ccc(-c3cccc(Nc4cc(NC(=O)C5CC5)ncc4C(C)=O)c3OC)cc21] | LC-MS calc. for C₃₆H₃₉N₆O₆S [M + H]⁺: m/z = 683.3; Found: 683.3. |
| 147 | N-(5-acetyl-4-((3-(1-ethyl- 4-(isoquinolin-1- ylmethyl)-2-oxo- 2,3,4,5-tetrahydro-1H- benzo[e][1,4]diazepin-8- yl)-2-methoxyphenyl)- amino)pyridin-2- yl)cyclopropanecarboxamide | 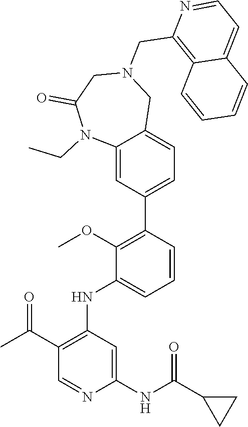 [SMILES: CCN1C(=O)CN(Cc2nccc3ccccc23)Cc2ccc(-c3cccc(Nc4cc(NC(=O)C5CC5)ncc4C(C)=O)c3OC)cc21] | LC-MS calc. for C₃₉H₃₉N₆O₄ [M + H]⁺: m/z = 655.3; Found: 655.4. |
| 148 | N-(5-acetyl-4-((3-(4-((4- cyanopyridin-2-yl)methyl)- 1-ethyl-2-oxo- 2,3,4,5-tetrahydro-1H- benzo[e][1,4]diazepin-8- yl)-2-methoxyphenyl)- amino)pyridin-2- yl)cyclopropanecarboxamide | 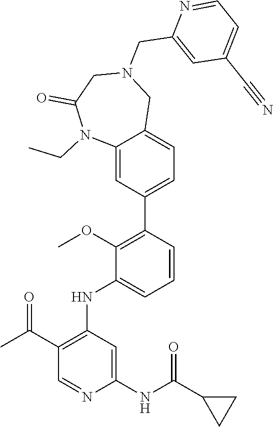 [SMILES: CCN1C(=O)CN(Cc2cc(C#N)ccn2)Cc2ccc(-c3cccc(Nc4cc(NC(=O)C5CC5)ncc4C(C)=O)c3OC)cc21] | LC-MS calc. for C₃₆H₃₆N₇O₄ [M + H]⁺: m/z = 630.3; Found: 630.2. |
| 149 | N-(5-acetyl-4-((3-(1-(2,2- difluoroethyl)-4- (imidazo[1,5-a]pyridin-8- ylmethyl)-2-oxo-2,3,4,5- tetrahydro-1H-pyrido[3,2- e][1,4]diazepin-8-yl)-2- methoxyphenyl)amino) pyridin-2-yl)cyclopropane- carboxamide | 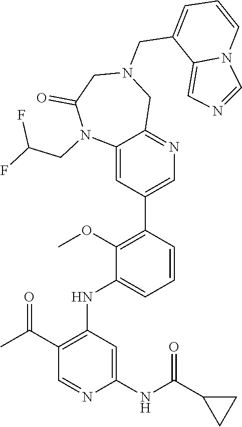 [SMILES: COc1c(Nc2cc(NC(=O)C3CC3)ncc2C(C)=O)cccc1-c1cnc2c(c1)N(CC(F)F)C(=O)CN(Cc1cccn3cncc13)C2] | LC-MS calc. for C₃₆H₃₅F₂N₈O₄ [M + H]⁺: m/z = 681.3; Found: 681.2. |
| 150 | N-(5-acetyl-4-((3-(1-(2,2- difluoroethyl)-4- (imidazo[1,2-a]pyridin-8- ylmethyl)-2-oxo-2,3,4,5- tetrahydro-1H-pyrido[3,2- e][1,4]diazepin-8-yl)-2- methoxyphenyl)amino) pyridin-2-yl)cyclopropane- carboxamide | 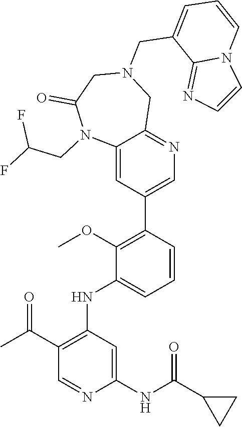 [SMILES: COc1c(Nc2cc(NC(=O)C3CC3)ncc2C(C)=O)cccc1-c1cnc2c(c1)N(CC(F)F)C(=O)CN(Cc1cccn3ccnc13)C2] | LC-MS calc. for C₃₆H₃₅F₂N₈O₄ [M + H]⁺: m/z = 681.3; Found: 681.2. |
| 151 | N-(5-acetyl-4-((3-(1-(2,2- difluoroethyl)-4-(3- (difluoromethyl)-2- fluorobenzyl)-2-oxo- 2,3,4,5-tetrahydro-1H- pyrido[3,2-e][1,4]diazepin- 8-yl)-2-methoxyphenyl)- amino)pyridin-2-yl)cyclo- propanecarboxamide | 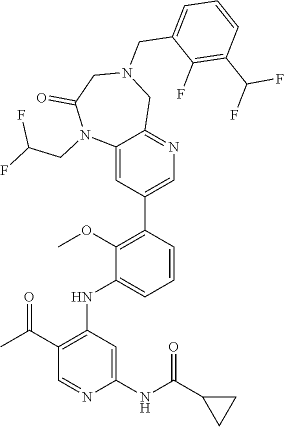 [SMILES: COc1c(Nc2cc(NC(=O)C3CC3)ncc2C(C)=O)cccc1-c1cnc2c(c1)N(CC(F)F)C(=O)CN(Cc1cccc(C(F)F)c1F)C2] | LC-MS calc. for C₃₆H₃₄F₅N₆O₄ [M + H]⁺: m/z = 709.3; Found: 709.3. |
| 152 | N-(5-acetyl-4-((3-(4-(3- chlorobenzyl)-1-(2,2- difluoroethyl)-2-oxo- 2,3,4,5-tetrahydro-1H- pyrido[3,2-e][1,4]diazepin- 8-yl)-2-methoxyphenyl)- amino)pyridin-2- yl)cyclopropane- carboxamide | 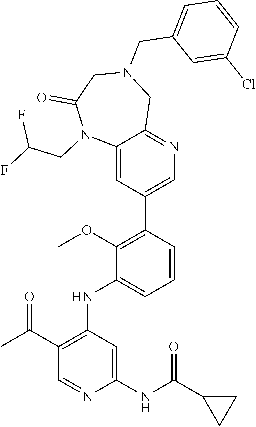 [SMILES: COc1c(Nc2cc(NC(=O)C3CC3)ncc2C(C)=O)cccc1-c1cnc2c(c1)N(CC(F)F)C(=O)CN(Cc1cccc(Cl)c1)C2] | LC-MS calc. for C₃₅H₃₄ClF₂N₆O₄ [M + H]⁺: m/z = 675.2; Found: 675.2. |
| 153 | N-(5-acetyl-4-((3-(1-(2,2- difluoroethyl)-4-((1- methyl-2-oxo-1,2- dihydropyridin-3- yl)methyl)-2-oxo-2,3,4,5- tetrahydro-1H-pyrido[3,2- e][1,4]diazepin-8-yl)-2- methoxyphenyl)amino) pyridin-2-yl)cyclopropane- carboxamide | 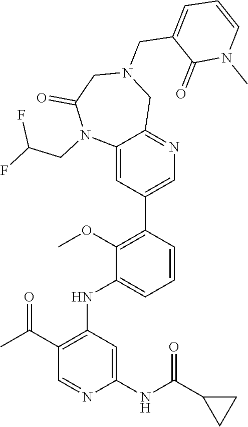 [SMILES: COc1c(Nc2cc(NC(=O)C3CC3)ncc2C(C)=O)cccc1-c1cnc2c(c1)N(CC(F)F)C(=O)CN(Cc1cccn(C)c1=O)C2] | LC-MS calc. for C₃₅H₃₆F₂N₇O₅ [M + H]⁺: m/z = 672.3; Found: 672.4. |
| 154 | N-(5-acetyl-4-((3-(1-(2,2- difluoroethyl)-2-oxo-4-((6- oxo-1,6-dihydropyridin-2- yl)methyl)-2,3,4,5- tetrahydro-1H-pyrido[3,2- e][1,4]diazepin-8-yl)-2- methoxyphenyl)amino) pyridin-2-yl)cyclopropane- carboxamide | 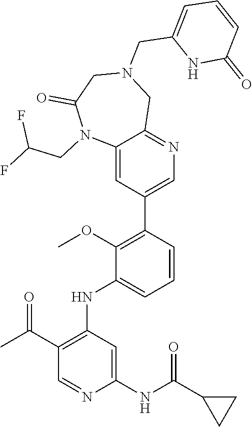 [SMILES: COc1c(Nc2cc(NC(=O)C3CC3)ncc2C(C)=O)cccc1-c1cnc2c(c1)N(CC(F)F)C(=O)CN(Cc1cccc(=O)[nH]1)C2] | LC-MS calc. for C₃₄H₃₄F₂N₇O₅ [M + H]⁺: m/z = 658.3; Found: 658.3. |
| 155 | N-(5-acetyl-4-((3-(1-(2,2- difluoroethyl)-4-((6- (difluoromethyl)pyridin-2- yl)methyl)-2-oxo-2,3,4,5- tetrahydro-1H-pyrido[3,2- e][1,4]diazepin-8-yl)-2- methoxyphenyl)amino) pyridin-2-yl)cyclopropane- carboxamide | 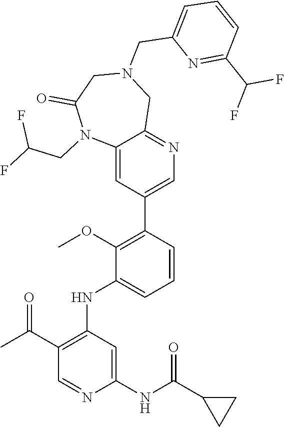 [SMILES: COc1c(Nc2cc(NC(=O)C3CC3)ncc2C(C)=O)cccc1-c1cnc2c(c1)N(CC(F)F)C(=O)CN(Cc1cccc(C(F)F)n1)C2] | LC-MS calc. for C₃₅H₃₄F₄N₇O₄ [M + H]⁺: m/z = 692.3; Found: 692.2. |
| 156 | N-(5-acetyl-4-((3-(1-(2,2- difluoroethyl)-2-oxo-4- (pyridin-2-ylmethyl)- 2,3,4,5-tetrahydro-1H- pyrido[3,2-e][1,4]diazepin- 8-yl)-5-fluoro-2- methoxyphenyl)amino) pyridin-2-yl)cyclopropane- carboxamide | 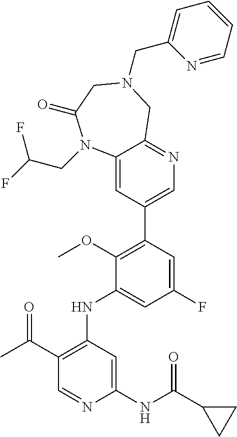 [SMILES: COc1c(Nc2cc(NC(=O)C3CC3)ncc2C(C)=O)cc(F)cc1-c1cnc2c(c1)N(CC(F)F)C(=O)CN(Cc1ccccn1)C2] | LC-MS calc. for C₃₄H₃₃F₃N₇O₄ [M + H]⁺: m/z = 660.3; Found: 660.2. |
| 157 | N-(5-acetyl-4-((3-(1-(2,2- difluoroethyl)-2-oxo-4- (pyridin-3-ylmethyl)- 2,3,4,5-tetrahydro-1H- pyrido[3,2-e][1,4]diazepin- 8-yl)-5-fluoro-2- methoxyphenyl)amino) pyridin-2-yl)cyclopropane- carboxamide | 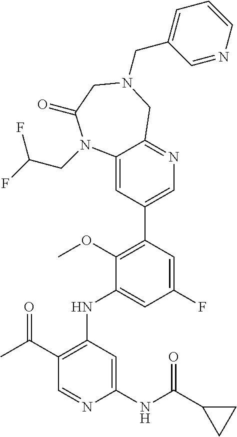 [SMILES: COc1c(Nc2cc(NC(=O)C3CC3)ncc2C(C)=O)cc(F)cc1-c1cnc2c(c1)N(CC(F)F)C(=O)CN(Cc1cccnc1)C2] | LC-MS calc. for C₃₄H₃₃F₃N₇O₄ [M + H]⁺: m/z = 660.3; Found: 660.1. |
| 158 | N-(5-acetyl-4-((3-(1-(2,2- difluoroethyl)-4-((6- (hydroxymethyl)pyridin-2- yl)methyl)-2-oxo-2,3,4,5- tetrahydro-1H-pyrido[3,2- e][1,4]diazepin-8-yl)-5- fluoro-2-methoxyphenyl)- amino)pyridin-2- yl)cyclopropane- carboxamide | 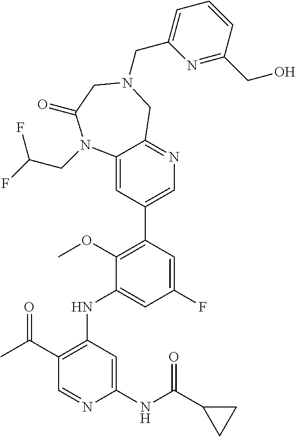 [SMILES: COc1c(Nc2cc(NC(=O)C3CC3)ncc2C(C)=O)cc(F)cc1-c1cnc2c(c1)N(CC(F)F)C(=O)CN(Cc1cccc(CO)n1)C2] | LC-MS calc. for C₃₅H₃₅F₃N₇O₅ [M + H]⁺: m/z = 690.3; Found: 690.2. |
| 159 | N-(5-acetyl-4-((3-(1-(2,2- difluoroethyl)-2-oxo-4- (thiazol-4-ylmethyl)- 2,3,4,5-tetrahydro-1H- pyrido[3,2-e][1,4] diazepin-8-yl)-5-fluoro-2- methoxyphenyl)amino) pyridin-2-yl)cyclopropane- carboxamide | 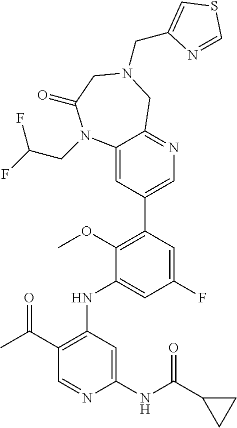 [SMILES: COc1c(Nc2cc(NC(=O)C3CC3)ncc2C(C)=O)cc(F)cc1-c1cnc2c(c1)N(CC(F)F)C(=O)CN(Cc1cscn1)C2] | LC-MS calc. for C₃₂H₃₁F₃N₇O₄S [M + H]⁺: m/z = 666.2; Found: 666.2. |

### Example 160. 6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methoxy-N-methylpicolinamide

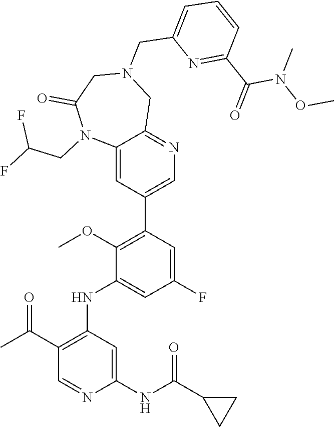 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)OC)n1)C2]

### Step 1. 6-formyl-N-methoxy-N-methylpicolinamide

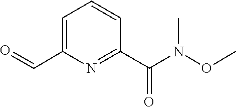

To a stirring suspension of 6-formylpicolinic acid (150 mg, 0.99 mmol) in DCM (3.3 mL) at 0° C. was added oxalyl chloride (102 μL, 1.19 mmol) and 1 drop of DMF. The reaction was allowed to warm to rt and stirred for 3 hours, then directly condensed at 30° C. The residue was taken up in 1 mL EtOAc and added to an ice cold, stirring, biphasic solution of potassium carbonate (274 mg, 1.99 mmol) in water (3.3 mL) and EtOAc (6.6 mL). The reaction was allowed to warm to rt and stirred vigorously overnight. The layers were separated, and the aqueous layer was extracted with EtOAc. The combined organics were dried and condensed to afford the title compound (193 mg, assumed quant. yield) which was carried forward without any further purification. LC-MS calc. for C₉H₁₁N₂O₃ [M+H]⁺: m/z=195.1; Found: 195.0.

### Step 2. 6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methoxy-N-methylpicolinamide

The title compound was prepared as a TFA salt in accordance with the synthetic protocols set forth in Example 138 Step 3 using the appropriate intermediates. LC-MS calc. for C₃₇H₃₈F₃N₈O₆ [M+H]⁺: m/z=747.3; Found: 747.1.

### Example 161. 3-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methoxy-N-methylbenzamide

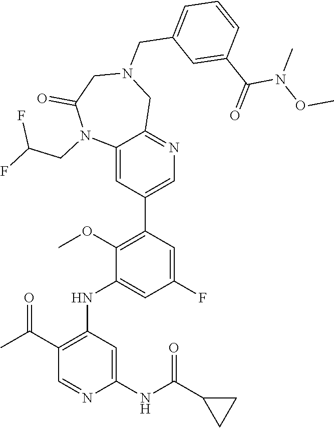 [SMILES: COc1c(Nc2cc(NC(=O)C3CC3)ncc2C(C)=O)cc(F)cc1-c1cnc2c(c1)N(CC(F)F)C(=O)CN(Cc1cccc(C(=O)N(C)OC)c1)C2]

The title compound was prepared as a TFA salt in accordance with the synthetic protocols set forth in Example 160 using the appropriate intermediates and commercially available starting materials. LC-MS calc. for C₃₈H₃₉F₃N₇O₆ [M+H]⁺: m/z=746.3; Found: 746.1.

### Example 162. N-(5-acetyl-4-((3-(4-((6-aminopyridin-2-yl)methyl)-1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide

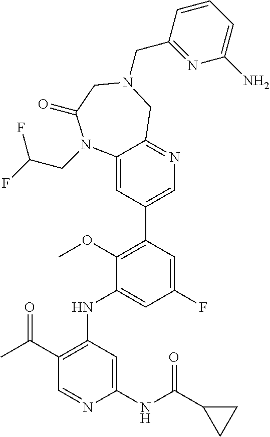 [SMILES: COc1c(Nc2cc(NC(=O)C3CC3)ncc2C(C)=O)cc(F)cc1-c1cnc2c(c1)N(CC(F)F)C(=O)CN(Cc1cccc(N)n1)C2]

### Step 1. tert-butyl (6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)pyridin-2-yl)carbamate

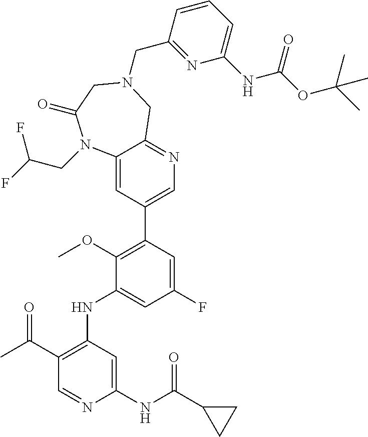 [SMILES: COc1c(Nc2cc(NC(=O)C3CC3)ncc2C(C)=O)cc(F)cc1-c1cnc2c(c1)N(CC(F)F)C(=O)CN(Cc1cccc(NC(=O)OC(C)(C)C)n1)C2]

The title compound was prepared in accordance with the synthetic protocols set forth in Example 138 Step 2 using the appropriate intermediates and commercial starting materials. LC-MS calc. for C₃₉H₄₂F₃N₈O₆ [M+H]⁺: m/z=775.3; Found: 775.2.

### Step 2. N-(5-acetyl-4-((3-(4-((6-aminopyridin-2-yl)methyl)-1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide

To a stirring solution of tert-butyl (6-((8-(3-((5-acetyl-2-(cyclopropanecarboxamido)-pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)pyridin-2-yl)carbamate (6.9 mg, 0.009 mmol) in 1,4-dioxane (50 μL) was added HCl (45 μL, 0.18 mmol, 4M in 1,4-dioxane) and the reaction was stirred at rt overnight. The reaction mixture was directly condensed, then taken up in MeOH and purified by prep-HPLC (CSH-C18, 18.6-38.6% MeCN/water (0.2% TFA) over 5 min) to afford the TFA salt of the title compound (1.9 mg, 21% yield) as a fluffy white powder. LC-MS calc. for C₃₄H₃₄F₃N₈O₄ [M+H]⁺: m/z=675.3; Found: 675.3.

### Example 163. N-(5-acetyl-4-((3-(1-(2,2-difluoroethyl)-4-((6-(3-methylureido)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide

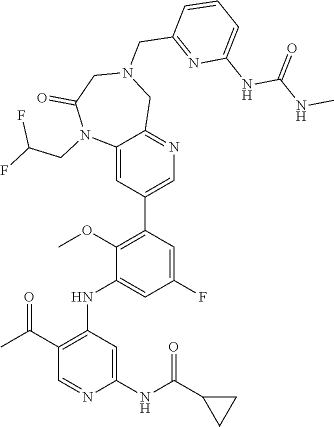 [SMILES: CNC(=O)Nc1cccc(CN2CC(=O)N(CC(F)F)c3cc(-c4cc(F)cc(Nc5cc(NC(=O)C6CC6)ncc5C(C)=O)c4OC)cnc3C2)n1]

### Step 1. N-(5-acetyl-4-((3-(4-((6-aminopyridin-2-yl)methyl)-1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide

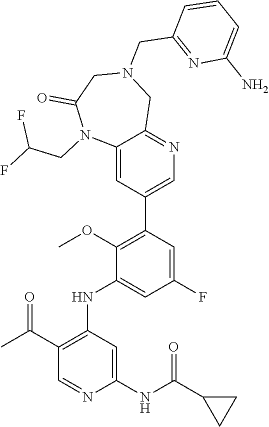 [SMILES: COc1c(Nc2cc(NC(=O)C3CC3)ncc2C(C)=O)cc(F)cc1-c1cnc2c(c1)N(CC(F)F)C(=O)CN(Cc1cccc(N)n1)C2]

To a stirring solution of tert-butyl (6-((8-(3-((5-acetyl-2-(cyclopropanecarboxamido)-pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)pyridin-2-yl)carbamate (64 mg, 0.086 mmol) in 1,4-dioxane (0.5 mL) was added HCl (0.41 mL, 1.65 mmol, 4M in 1,4-dioxane) and the reaction was stirred at rt overnight. The reaction mixture was directly condensed to afford the HCl salt of the title compound (62 mg, quant yield). The material was used crude without purification. LC-MS calc. for C₃₄H₃₄F₃N₈O₄[M+H]⁺: m/z=675.3; Found: 675.3.

### Step 2. N-(5-acetyl-4-((3-(1-(2,2-difluoroethyl)-4-((6-(3-methylureido)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide

To a stirring solution of N-(5-acetyl-4-((3-(4-((6-aminopyridin-2-yl)methyl)-1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide HCl (10 mg, 0.013 mmol) in pyridine (0.10 mL) was added methylcarbamic chloride (9.8 μL, 0.128 mmol). The reaction was stirred at rt for 3 hours before another portion of methylcarbamic chloride (9.8 μL, 0.128 mmol) was added, and the reaction was stirred at rt for 6 days. The reaction mixture was diluted in MeOH and purified by prep-LCMS (CSH-C18, 19.6-39.6% MeCN/water (0.2% TFA) over 5 min) to afford the title compound (1.4 mg, 10% yield) as a fluffy white powder. LC-MS calc. for C₃₆H₃₇F₃N₉O₅ [M+H]⁺: m/z=732.3; Found: 732.3.

### Example 164. N-(5-acetyl-4-((3-(1-(2,2-difluoroethyl)-4-((6-((N-methylsulfamoyl)amino)-pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide

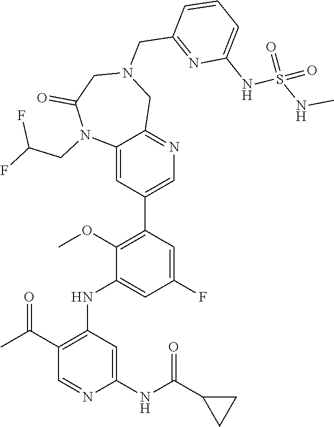 [SMILES: CNS(=O)(=O)Nc1cccc(CN2CC(=O)N(CC(F)F)c3cc(-c4cc(F)cc(Nc5cc(NC(=O)C6CC6)ncc5C(C)=O)c4OC)cnc3C2)n1]

To a stirring solution of N-(5-acetyl-4-((3-(4-((6-aminopyridin-2-yl)methyl)-1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide HCl (7 mg, 0.009 mmol) in pyridine (0.1 mL) and DCM (0.25 mL) was added methylsulfamoyl chloride (1 drop). The reaction was stirred at rt for 1 hour, then diluted with MeOH and TFA and purified on prep-LCMS (CSH-C18, 18.7-38.7% MeCN/water (0.2% TFA) over 5 min) to afford the TFA salt of the title compound (1.3 mg, 13% yield). LC-MS calc. for C₃₅H₃₇F₃N₉O₆S [M+H]⁺: m/z=768.3; Found: 768.2.

### Example 165. N-(5-Acetyl-4-((2-methoxy-3-(1-methyl-2-oxo-4-((6-(tetrahydrofuran-2-yl)pyridin-2-yl)methyl)-2,3,4,5-tetrahydro-1H-benzo[e][1,4]diazepin-8-yl)phenyl)amino)pyridin-2-yl)cyclopropanecarboxamide

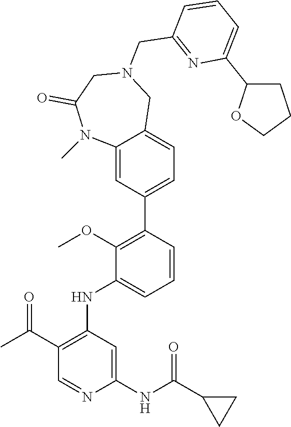 [SMILES: COc1c(Nc2cc(NC(=O)C3CC3)ncc2C(C)=O)cccc1-c1ccc2c(c1)N(C)C(=O)CN(Cc1cccc(C3CCCO3)n1)C2]

### Step 1. tert-Butyl 8-bromo-2-oxo-1,2,3,5-tetrahydro-4H-benzo[e][1,4]diazepine-4-carboxylate

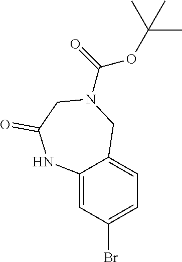 [SMILES: CC(C)(C)OC(=O)N1CC(=O)Nc2cc(Br)ccc2C1]

Di-tert-butyl decarbonate (95 μL, 0.41 mmol) was added to a stirring solution of 8-bromo-1,3,4,5-tetrahydro-2H-benzo[e][1,4]diazepin-2-one (Int. 25) (100 mg, 0.28 mmol) and N,N-diisopropylethylamine (98 μL, 0.56 mmol) in THF (2 mL). The reaction mixture was stirred for 1.5 hours. The product mixture was diluted with EtOAc (50 mL) and washed with a sat. NH₄Cl (aq) solution and brine (50 mL). The organic layer was dried over Na₂SO₄, filtered and concentrated under reduced pressure to yield the title compound (96 mg, 100% yield) which was used without further purification. LC-MS calc. for C₁₀H₁₀BrN₂O₃[M+H—C₄H₈]⁺: m/z=285.0/287.0; Found: 284.9/286.9.

### Step 2. tert-Butyl 8-bromo-1-methyl-2-oxo-1,2,3,5-tetrahydro-4H-benzo[e][1,4]-diazepine-4-carboxylate

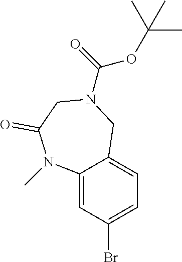 [SMILES: CN1C(=O)CN(C(=O)OC(C)(C)C)Cc2ccc(Br)cc21]

Iodomethane (19 μL, 0.31 mmol) was added to a stirring solution of tert-butyl 8-bromo-2-oxo-1,2,3,5-tetrahydro-4H-benzo[e][1,4]diazepine-4-carboxylate (70 mg, 0.21 mmol) and cesium carbonate (134 mg, 0.41 mmol) in DMF (1 mL). The reaction mixture was stirred at rt for 4 hours. The product mixture was diluted with EtOAc (30 mL) and washed with brine (30 mL). The organic layer was dried over Na₂SO₄, filtered and concentrated under reduced pressure. The residue obtained was purified by silica gel column chromatography (0-100% EtOAc/Hexane) to yield the title compound (48 mg, 66% yield). LC-MS calc. for C₁₁H₁₂BrN₂O₃[M+H—C₄H₈]⁺: m/z=299.0/301.0; Found: 298.9/300.9.

### Step 3. 8-Bromo-1-methyl-1,3,4,5-tetrahydro-2H-benzo[e][1,4]diazepin-2-one

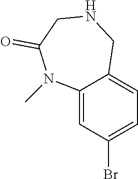

Hydrochloric acid (0.34 mL, 1.4 mmol, 4.0 M in 1,4-dioxane) was added to a stirring solution of tert-butyl 8-bromo-1-methyl-2-oxo-1,2,3,5-tetrahydro-4H-benzo[e][1,4]diazepine-4-carboxylate (48 mg, 0.14 mmol) in DCM (2 mL). The reaction mixture was stirred for 4 hours. The product mixture was concentrated under reduced pressure to obtain the title compound as the HCl salt (39 mg, 99% yield). LC-MS calc. for C₁₀H₁₂BrN₂O [M+H]⁺: m/z=255.0/257.0; Found: 254.9/256.9.

### Step 4. 6-(Furan-2-yl)picolinaldehyde

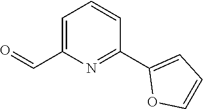 [SMILES: O=Cc1cccc(-c2ccco2)n1]

A 20 mL scintillation vial was charged with 6-bromopicolinaldehyde (200 mg, 1.1 mmol), furan-2-ylboronic acid (180 mg, 1.6 mmol), potassium carbonate (446 mg, 3.2 mmol), and [1,1′-bis(diphenylphosphino)ferrocene]dichloropalladium(II) complex with dichloromethane (44 mg, 0.05 mmol). The mixture was dissolved in 1,4-dioxane (5 mL) and water (1 mL), was sparged with nitrogen gas for 5 minutes and sealed. Then the reaction mixture was heated to 80° C. for 3 hours. The product mixture was diluted with EtOAc (60 mL) and washed with sat. NaHCO₃ (aq) solution (60 mL) and then brine (60 mL). The organic layer was dried over Na₂SO₄, filtered and concentrated under reduced pressure. The residue obtained was purified by silica gel column chromatography (0-5% MeOH/DCM) to yield the title compound (170 mg, 91% yield). LC-MS calc. for C₁₀H₈NO₂ [M+H]⁺: m/z=174.1; Found: 174.0.

### Step 5. (6-(Tetrahydrofuran-2-yl)pyridin-2-yl)methanol

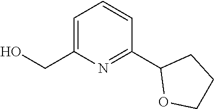 [SMILES: OCc1cccc(C2CCCO2)n1]

A 40 mL scintillation vial was charged with 6-(furan-2-yl)picolinaldehyde (170 mg, 0.98 mmol) and palladium on carbon (313 mg, 0.29 mmol, 10% wt). The mixture was dissolved in MeOH (4 mL) and placed under an atmosphere of hydrogen gas. The reaction mixture was heated to 50° C. and stirred overnight. The product mixture was filtered through celite and concentrated under reduced pressure to yield the title compound (160 mg, 91% yield). LC-MS calc. for C₁₀H₁₄NO₂ [M+H]⁺: m/z=180.1; Found: 180.0.

### Step 6. (6-(Tetrahydrofuran-2-yl)pyridin-2-yl)methyl methanesulfonate

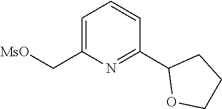 [SMILES: CS(=O)(=O)OCc1cccc(C2CCCO2)n1]

Methanesulfonyl chloride (52 μL, 0.67 mmol) was added to a stirring solution of (6-(tetrahydrofuran-2-yl)pyridin-2-yl)methanol (80 mg, 0.45 mmol) and triethylamine (187 μL, 1.3 mmol) in DCM (3 mL) at 0° C. The reaction mixture was stirred for 1 hour. The product mixture was diluted with sat. NaHCO₃ (aq) (30 mL) and extracted with DCM (2×30 mL). The combined organic layers were dried over Na₂SO₄, filtered and concentrated under reduced pressure. The residue obtained was purified by silica gel column chromatography (0-90% EtOAc/Hexane) to yield the title compound (63 mg, 55% yield). LC-MS calc. for C₁₁H₁₆NO₄S [M+H]⁺: m/z=258.1; Found: 258.0.

### Step 7. 8-Bromo-1-methyl-4-((6-(tetrahydrofuran-2-yl)pyridin-2-yl)methyl)-1,3,4,5-tetrahydro-2H-benzo[e][1,4]diazepin-2-one

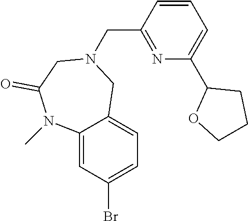 [SMILES: CN1C(=O)CN(Cc2cccc(C3CCCO3)n2)Cc2ccc(Br)cc21]

Potassium carbonate (55 mg, 0.40 mmol) was added to a stirring solution of (6-(tetrahydrofuran-2-yl)pyridin-2-yl)methyl methanesulfonate (52 mg, 0.20 mmol) and 8-bromo-1-methyl-1,3,4,5-tetrahydro-2H-benzo[e][1,4]diazepin-2-one hydrochloride (39 mg, 0.13 mmol) in DMF (1 mL). The reaction mixture was stirred overnight. The product mixture was diluted with MeCN (3 mL), filtered and purified directly by prep-HPLC on a C₁₈ column (17.2-37.2% MeCN in 0.2% TFA(aq)) to afford the TFA salt of the title compound (84 mg, 97% yield). LC-MS calc. for C₂₀H₂₃BrN₃O₂[M+H]⁺: m/z=416.1/418.1; Found: 416.0/418.1.

### Step 8. N-(5-acetyl-4-((2-methoxy-3-(1-methyl-2-oxo-4-((6-(tetrahydrofuran-2-yl)pyridin-2-yl)methyl)-2,3,4,5-tetrahydro-1H-benzo[e][1,4]diazepin-8-yl)phenyl)amino)pyridin-2-yl)cyclopropanecarboxamide

The title compound was prepared in accordance with the synthetic protocols set forth in Example 36 Step 2 using appropriate intermediates, as well as commercial starting materials. LC-MS calc. for C₃₈H₄₁N₆O₅ [M+H]⁺: m/z=661.3; Found: 661.3.

### Example 166. N-(5-Acetyl-4-((2-methoxy-3-(4-((6-(3-methoxyoxetan-3-yl)pyridin-2-yl)methyl)-1-methyl-2-oxo-2,3,4,5-tetrahydro-1H-benzo[e][1,4]diazepin-8-yl)phenyl)amino)pyridin-2-yl)cyclopropanecarboxamide

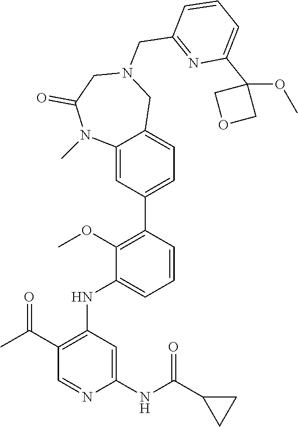 [SMILES: COc1c(Nc2cc(NC(=O)C3CC3)ncc2C(C)=O)cccc1-c1ccc2c(c1)N(C)C(=O)CN(Cc1cccc(C3(OC)COC3)n1)C2]

### Step 1. 2-Bromo-6-(((tert-butyldimethylsilyl)oxy)methyl)pyridine

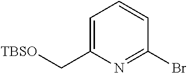 [SMILES: CC(C)(C)[Si](C)(C)OCc1cccc(Br)n1]

tert-Butyldimethylsilyl chloride (601 mg, 4.0 mmol) was added to a stirring solution of imidazole (362 mg, 5.3 mmol) and (6-bromopyridin-2-yl)methanol (500 mg, 2.7 mmol) in DCM (10 mL). The reaction mixture was stirred at rt for 2 hours. The product mixture was diluted with EtOAc (60 mL) and washed with water (60 mL) then brine (60 mL). The organic layer was dried over Na₂SO₄, filtered and concentrated under reduced pressure. The residue obtained was purified by silica gel column chromatography (0-30% EtOAc/Hexane) to yield the title compound (765 mg, 95% yield). LC-MS calc. for C₁₂H₂₁BrNOSi [M+H]⁺: m/z=302.1/304.1; Found: 302.0/303.9.

### Step 2. 3-(6-(((tert-Butyldimethylsilyl)oxy)methyl)pyridin-2-yl)oxetan-3-ol

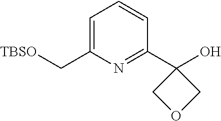 [SMILES: CC(C)(C)[Si](C)(C)OCc1cccc(C2(O)COC2)n1]

n-Butyllithium (970 μL, 2.4 mmol, 2.5 M in hexanes) was added to a stirring solution of 2-bromo-6-(((tert-butyldimethylsilyl)oxy)methyl)pyridine (665 mg, 2.2 mmol) in THF (11 mL) at −78° C. The reaction mixture was stirred for 10 minutes then a solution of oxetan-3-one (210 μL, 3.3 mmol) in THF (5 mL) was added to the reaction mixture. The reaction mixture was stirred for 2 hours and allowed to warm to rt. The product mixture was diluted with sat. NaHCO₃ (aq) solution (60 mL) and extracted with EtOAc (60 mL). The organic layer was washed with brine (60 mL), dried over Na₂SO₄, filtered and concentrated under reduced pressure. The residue obtained was purified by silica gel column chromatography (0-100% EtOAc/Hexane) to yield the title compound (473 mg, 73% yield). LC-MS calc. for C₁₅H₂₆NO₃Si [M+H]⁺: m/z=296.2; Found: 296.1.

### Step 3. 2-(((tert-Butyldimethylsilyl)oxy)methyl)-6-(3-methoxyoxetan-3-yl)pyridine

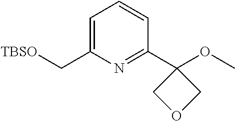 [SMILES: COC1(c2cccc(CO[Si](C)(C)C(C)(C)C)n2)COC1]

Sodium hydride (16 mg, 0.41 mmol, 60% dispersion in mineral oil) was added to a stirring solution of 3-(6-(((tert-butyldimethylsilyl)oxy)methyl)pyridin-2-yl)oxetan-3-ol (100 mg, 0.34 mmol) in THF (3 mL) at 0° C. The reaction mixture was stirred for 5 minutes then iodomethane (32 μL, 0.51 mmol) was added. The reaction mixture was warmed to rt and stirred for 6 hours. The product mixture was diluted with water (30 mL) and extracted with EtOAc (30 mL). The organic layer was washed with brine (30 mL), dried over Na₂SO₄, filtered and concentrated under reduced pressure. The residue obtained was purified by silica gel column chromatography (0-100% EtOAc/Hexane) to yield the title compound (91 mg, 87% yield). LC-MS calc. for C₁₆H₂₈NO₃Si [M+H]⁺: m/z=310.2; Found: 310.1.

### Step 4. 6-(3-Methoxyoxetan-3-yl)picolinaldehyde

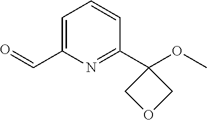

Tetrabutylammonium fluoride (440 μL, 0.44 mmol, 1.0 M in THF) was added to a stirring solution of 2-(((tert-butyldimethylsilyl)oxy)methyl)-6-(3-methoxyoxetan-3-yl)pyridine (91 mg, 0.29 mmol) in THF (2.7 mL) at 0° C. The reaction mixture was stirred for 2 hours at 0° C. The product mixture was diluted with sat. NaHCO₃ (aq) (30 mL) and extracted with a 3:1 CHCl₃/iPrOH mixture (5×30 mL). The combined organic layers were dried over MgSO₄, filtered and concentrated under reduced pressure. The residue obtained was dissolved in DCM (2.7 mL). The solution was cooled to 0° C. and Dess-Martin periodinane (172 mg, 0.41 mmol) was added. The reaction mixture was stirred at rt for 2 hours. The product mixture was diluted with sat. NaHCO₃ (aq) (30 mL) and extracted with a 3:1 CHCl₃/iPrOH mixture (3×30 mL). The combined organic layers were dried over MgSO₄, filtered and concentrated under reduced pressure. The residue obtained was purified by silica gel column chromatography (0-10% MeOH/DCM) to yield the title compound (31 mg, 55% yield). LC-MS calc. for C₁₀H₁₂NO₃ [M+H]⁺: m/z=194.1; Found: 194.0.

### Step 5. 8-Bromo-4-((6-(3-methoxyoxetan-3-yl)pyridin-2-yl)methyl)-1-methyl-1,3,4,5-tetrahydro-2H-benzo[e][1,4]diazepin-2-one

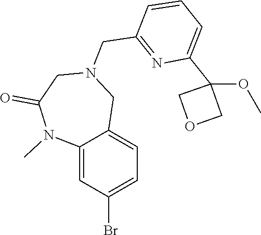 [SMILES: COC1(c2cccc(CN3CC(=O)N(C)c4cc(Br)ccc4C3)n2)COC1]

Sodium triacetoxyborohydride (44 mg, 21 μmol) was added to a stirring solution of 6-(3-methoxyoxetan-3-yl)picolinaldehyde (31 mg, 150 μmol) and 8-bromo-1-methyl-1,3,4,5-tetrahydro-2H-benzo[e][1,4]diazepin-2-one hydrochloride (20 mg, 69 μmol) in DCM (1 mL). The reaction mixture was heated to 35° C. and stirred for 4 hours. The product mixture was diluted with DMSO (3 mL) and directly purified by prep-HPLC on a C₁₈ column (17.7-37.7% MeCN in 0.2% TFA(aq)) to afford the TFA salt of the title compound (15 mg, 40% yield). LC-MS calc. for C₂₀H₂₃BrN₃O₃ [M+H]⁺: m/z=432.1/434.1; Found: 432.0/434.0.

### Step 6. N-(5-acetyl-4-((2-methoxy-3-(4-((6-(3-methoxyoxetan-3-yl)pyridin-2-yl)methyl)-1-methyl-2-oxo-2,3,4,5-tetrahydro-1H-benzo[e][1,4]diazepin-8-yl)phenyl)amino)pyridin-2-yl)cyclopropanecarboxamide

The title compound was prepared in accordance with the synthetic protocols set forth in Example 84 Step 2 using appropriate intermediates, as well as commercial starting materials. LC-MS calc. for C₃₈H₄₁N₆O₆ [M+H]⁺: m/z=677.3; Found: 677.3.

### Example 167. N-(5-Acetyl-4-((3-(4-((6-acetylpyridin-2-yl)methyl)-1-methyl-2-oxo-2,3,4,5-tetrahydro-1H-benzo[e][1,4]diazepin-8-yl)-2-methoxyphenyl)amino)pyridin-2-yl)cyclopropanecarboxamide

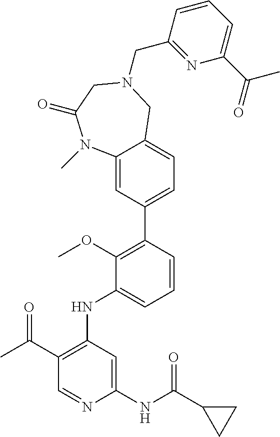 [SMILES: COc1c(Nc2cc(NC(=O)C3CC3)ncc2C(C)=O)cccc1-c1ccc2c(c1)N(C)C(=O)CN(Cc1cccc(C(C)=O)n1)C2]

### Step 1. 1-(6-(Bromomethyl)pyridin-2-yl)ethan-1-one

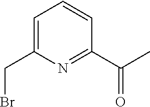

A 40 mL scintillation vial was charged with 1-(6-methylpyridin-2-yl)ethan-1-one (380 mg, 2.8 mmol), N-bromosuccinimide (494 mg, 2.8 mmol), and azobisisobutyronitrile (23 mg, 140 μmmol). The mixture was dissolved in CCl₄ (8 mL), sparged with nitrogen gas for 5 minutes, and sealed. The reaction mixture was heated to 70° C. and stirred for 12 hours. The product mixture was diluted with EtOAc (50 mL) and washed with water (50 mL) then brine (50 mL). The organic layer was dried over Na₂SO₄, filtered and concentrated under reduced pressure. The residue obtained was purified by silica gel column chromatography (0-100% EtOAc/Hexane) to yield the title compound (381 mg, 63% yield). LC-MS calc. for C₈H₈BrNO [M+H]⁺: m/z=214.0/216.0; Found: 213.9/215.9.

### Step 2. 4-((6-Acetylpyridin-2-yl)methyl)-8-bromo-1-methyl-1,3,4,5-tetrahydro-2H-benzo[e][1,4]diazepin-2-one

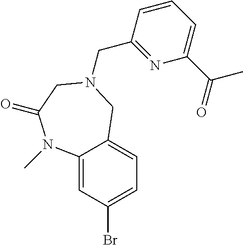 [SMILES: CC(=O)c1cccc(CN2CC(=O)N(C)c3cc(Br)ccc3C2)n1]

Potassium carbonate (33 mg, 0.24 mmol) was added to a stirring solution of 8-bromo-1-methyl-1,3,4,5-tetrahydro-2H-benzo[e][1,4]diazepin-2-one hydrochloride (23 mg, 79 μmol) and 1-(6-(bromomethyl)pyridin-2-yl)ethan-1-one (20 mg, 91 μmol) in MeCN (1 mL). The reaction mixture was heated to 50° C. and stirred for 4 hours. The product mixture was diluted with water (30 mL) and extracted with a 3:1 CHCl₃/iPrOH mixture (3×100 mL). The combined organic layers were dried over MgSO₄, filtered and concentrated under reduced pressure. The residue obtained was purified by silica gel column chromatography (0-5% MeOH/DCM) to yield the title compound (15 mg, 49% yield). LC-MS calc. for C₁₈H₁₉BrN₃O₂[M+H]⁺: m/z=388.1/390.1; Found: 388.0/390.0.

### Step 3. N-(5-Acetyl-4-((3-(4-((6-acetylpyridin-2-yl)methyl)-1-methyl-2-oxo-2,3,4,5-tetrahydro-1H-benzo[e][1,4]diazepin-8-yl)-2-methoxyphenyl)amino)pyridin-2-yl)cyclopropanecarboxamide

The title compound was prepared in accordance with the synthetic protocols set forth in Example 84 Step 2 using appropriate intermediates, as well as commercial starting materials. LC-MS calc. for C₃₆H₃₇N₆O₅ [M+H]⁺: m/z=633.3; Found: 633.3.

### Example 168. 6-(2-(8-(3-((5-Acetyl-2-(cyclopropanecarboxamido)pyridin-4-yl)amino)-2-methoxyphenyl)-1-methyl-2-oxo-1,2,3,5-tetrahydro-4H-benzo[e][1,4]diazepin-4-yl)propan-2-yl)-N,N-dimethylpicolinamide

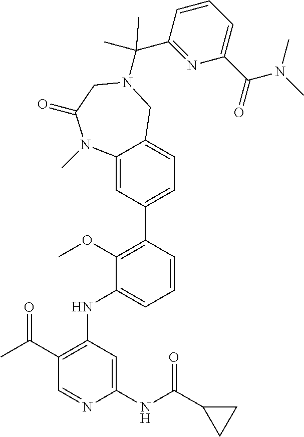 [SMILES: COc1c(Nc2cc(NC(=O)C3CC3)ncc2C(C)=O)cccc1-c1ccc2c(c1)N(C)C(=O)CN(C(C)(C)c1cccc(C(=O)N(C)C)n1)C2]

### Step 1. Methyl 6-(2-cyanopropan-2-yl)picolinate

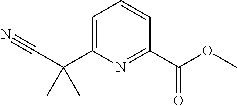

MeCN (1.6 mL, 31 mmol) was added to a stirring solution of n-butyllithium (10.7 mL, 26.7 mmol, 2.5 M in hexanes) in THF (17 mL) at −78° C. The reaction mixture was stirred for 30 minutes then 6-chloropicolinic acid (700 mg, 4.4 mmol) in THF (5 mL) was added. The reaction mixture was stirred for 1 hour at −78° C. then 1 hour at rt. The product mixture was filtered, and the precipitate was collected. The precipitate was transferred to a separatory funnel containing 1 N HCl (aq) (50 mL) and DCM (50 mL). The diluted product was extracted with DCM (2×50 mL), and the combined organic layers were dried over Na₂SO₄, filtered and concentrated under reduced pressure. The residue obtained was dissolved in DMF (17 mL) and cooled to 0° C. Sodium hydride (533 mg, 22.2 mmol, 60% dispersion in mineral oil) was added to the reaction mixture, and the reaction mixture was stirred for 10 minutes at 0° C. Then iodomethane (2.77 mL, 44.4 mmol) was added to the reaction mixture. The reaction mixture was warmed to rt and stirred for 1 hour. The product mixture was diluted with EtOAc (50 mL) and washed with water (50 mL) then brine (50 mL). The organic layer was dried over Na₂SO₄, filtered and concentrated under reduced pressure. The residue obtained was purified by silica gel column chromatography (0-100% EtOAc/Hexane) to yield the title compound (814 mg, 90% yield). LC-MS calc. for C₁₁H₁₃N₂O₂ [M+H]⁺: m/z=205.1; Found: 205.1.

### Step 2. 6-(1-Amino-2-methyl-1-oxopropan-2-yl)picolinic acid

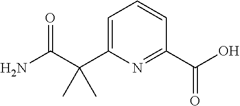

A solution of hydrogen peroxide-urea (1875 mg, 19.9 mmol) and sodium hydroxide (1594 mg, 39.9 mmol) in water (10 mL) was added to a stirring solution of methyl 6-(2-cyanopropan-2-yl)picolinate (814 mg, 3.99 mmol) in EtOH (10 mL). The reaction mixture was stirred 12 hours. The product mixture was diluted with MeCN (10 mL) and trifluoroacetic acid (1 mL) and purified directly by prep-HPLC on a C₁₈ column (5.1-25.1% MeCN in 0.2% TFA(aq)) to afford the TFA salt of the title compound (977 mg, 76% yield). LC-MS calc. for C₁₀H₁₃N₂O₃ [M+H]⁺: m/z=209.1; Found: 209.1.

### Step 3. 6-(1-Amino-2-methyl-1-oxopropan-2-yl)-N,N-dimethylpicolinamide

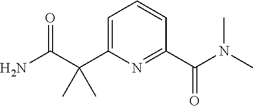 [SMILES: CN(C)C(=O)c1cccc(C(C)(C)C(N)=O)n1]

1-[Bis(dimethylamino)methylene]-1H-1,2,3-triazolo[4,5-b]pyridinium 3-oxid hexafluorophosphate (1.5 g, 3.9 mmol) was added to a stirring solution of N,N-diisopropylethylamine (3.17 mL, 18.2 mmol), dimethylamine hydrochloride (989 mg, 12.1 mmol) and the TFA salt of 6-(1-amino-2-methyl-1-oxopropan-2-yl)picolinic acid (977 mg, 3.0 mmol) in DMF (12 mL) at 0° C. The reaction mixture was stirred for 1 hour at 0° C. The product mixture was diluted with MeOH (5 mL) and purified directly by prep-HPLC on a C₁₈ column (7.0-27.0% MeCN in 0.2% TFA(aq)) to afford the TFA salt of the title compound (807 mg, 76% yield). LC-MS calc. for C₁₂H₁₈N₃O₂ [M+H]⁺: m/z=236.1; Found: 236.1.

### Step 4. 6-(2-Aminopropan-2-yl)-N,N-dimethylpicolinamide

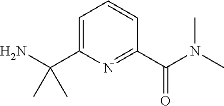

[Bis(trifluoroacetoxy)iodo]benzene (1192 mg, 2.77 mmol) was added to a stirring solution of the TFA salt of 6-(1-amino-2-methyl-1-oxopropan-2-yl)-N,N-dimethylpicolinamide (807 mg, 2.31 mmol) in MeCN (10 mL) and water (10 mL). The reaction was stirred at rt overnight. Then sodium hydroxide (462 mg, 11.6 mmol) was added, and the reaction mixture was stirred an additional 1 hour. The product mixture was diluted with MeCN (7 mL) and TFA (3 mL) and purified directly by prep-HPLC on a C₁₈ column (5.0-25.0% MeCN in 0.2% TFA(aq)) to afford the TFA salt of the title compound (380 mg, 51% yield). LC-MS calc. for C₁₁H₁₈N₃O [M+H]⁺: m/z=208.1; Found: 208.1.

### Step 5. 6-(2-((4-bromo-2-nitrobenzyl)amino)propan-2-yl)-N,N-dimethylpicolinamide

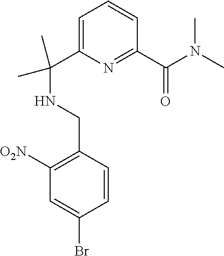 [SMILES: CN(C)C(=O)c1cccc(C(C)(C)NCc2ccc(Br)cc2[N+](=O)[O-])n1]

4-bromo-1-(bromomethyl)-2-nitrobenzene (236 mg, 0.80 mmol) was added to a stirring solution of potassium carbonate (492 mg, 3.6 mmol) and the TFA salt of 6-(2-aminopropan-2-yl)-N,N-dimethylpicolinamide (286 mg, 0.89 mmol) in DMF (4 mL) at rt. The reaction mixture was stirred for 2 hours. The product mixture was diluted with water (30 mL) and extracted with DCM (3×30 mL). The combined organic layers were dried over Na₂SO₄, filtered and concentrated under reduced pressure. The residue obtained was purified by silica gel column chromatography (0-5% MeOH/DCM) to yield the title compound (110 mg, 29% yield). LC-MS calc. for C₁₈H₂₂BrN₄O₃ [M+H]⁺: m/z=421.1; Found: 421.1.

### Step 6. Methyl N-(4-bromo-2-nitrobenzyl)-N-(2-(6-(dimethylcarbamoyl)pyridin-2-yl)propan-2-yl)glycinate

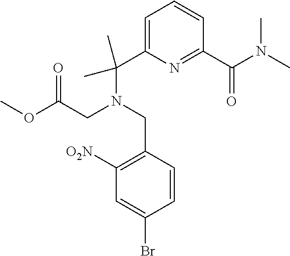

Methyl bromoacetate (33 μL, 0.34 mmol) was added to a stirring solution of 6-(2-((4-bromo-2-nitrobenzyl)amino)propan-2-yl)-N,N-dimethylpicolinamide (110 mg, 0.26 mmol) and potassium carbonate (72 mg, 0.52 mmol) in DMF (3 mL). The reaction mixture was heated to 85° C. and stirred for 4 hours. Another portion of methyl bromoacetate (33 μL, 0.34 mmol) was added and the reaction mixture was stirred another 4 hours at 85° C. The product mixture was diluted with EtOAc (30 mL) and washed with water (30 mL) and brine (2×30 mL). The organic layer was dried over Na₂SO₄, filtered and concentrated under reduced pressure. The residue obtained was purified by silica gel column chromatography (0-100% EtOAc/Hexane) to yield the title compound (71 mg, 55% yield). LC-MS calc. for C₂₁H₂₆BrN₄O₅[M+H]⁺: m/z=493.1; Found: 493.1.

### Step 7. 6-(2-(8-Bromo-2-oxo-1,2,3,5-tetrahydro-4H-benzo[e][1, 4]diazepin-4-yl)propan-2-yl)-N,N-dimethylpicolinamide

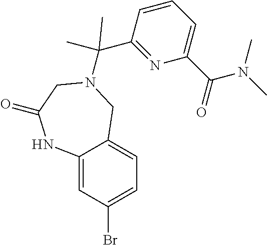 [SMILES: CN(C)C(=O)c1cccc(C(C)(C)N2CC(=O)Nc3cc(Br)ccc3C2)n1]

Iron powder (44 mg, 0.79 mmol) and ammonium chloride (42 mg, 0.79 mmol) were added to a stirring solution of methyl N-(4-bromo-2-nitrobenzyl)-N-(2-(6-(dimethylcarbamoyl)-pyridin-2-yl)propan-2-yl)glycinate (71 mg, 0.16 mmol) in EtOH (1.2 mL) and water (300 μL). The reaction mixture was heated to 80° C. and stirred for 1 hour. The product mixture was diluted with EtOAc (30 mL) and washed with water (30 mL) and brine (30 mL). The organic layer was dried over Na₂SO₄, filtered and concentrated under reduced pressure. The residue obtained was dissolved in THF (3 mL). Sodium hydride (19 mg, 0.47 mmol, 60% dispersion in mineral oil) was added, and the reaction mixture was heated to 60° C. and stirred for 3 hours. The product mixture was diluted with sat. NH₄Cl (aq) (30 mL) and extracted with a 3:1 CHCl₃/iPrOH mixture (3×30 mL). The combined organic layers were dried over MgSO₄, filtered and concentrated under reduced pressure to yield the title compound (43 mg, 63% yield). LC-MS calc. for C₂₀H₂₄BrN₄O₂[M+H]⁺: m/z=431.1/433.1; Found: 431.2/433.2.

### Step 8. 6-(2-(8-bromo-1-methyl-2-oxo-1,2,3,5-tetrahydro-4H-benzo[e][1,4]diazepin-4-yl)propan-2-yl)-N,N-dimethylpicolinamide

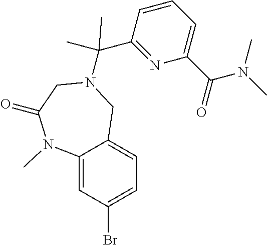 [SMILES: CN(C)C(=O)c1cccc(C(C)(C)N2CC(=O)N(C)c3cc(Br)ccc3C2)n1]

Iodomethane (4.3 μL, 70 μmol) was added to a stirring solution of cesium carbonate (34 mg, 104 μmol) and 6-(2-(8-bromo-2-oxo-1,2,3,5-tetrahydro-4H-benzo[e][1,4]diazepin-4-yl)propan-2-yl)-N,N-dimethylpicolinamide (15 mg, 35 μmol) in DMF (500 μL) at rt. The reaction mixture was stirred for 2 hours. The product mixture was purified directly by silica gel column chromatography (0-10% MeOH/DCM) to yield the title compound (7 mg, 45% yield). LC-MS calc. for C₂₁H₂₆BrN₄O₂[M+H]⁺: m/z=445.1/447.1; Found: 445.1/447.1.

### Step 9. 6-(2-(8-(3-((5-Acetyl-2-(cyclopropanecarboxamido)pyridin-4-yl)amino)-2-methoxyphenyl)-1-methyl-2-oxo-1,2,3,5-tetrahydro-4H-benzo[e][1,4]diazepin-4-yl)propan-2-yl)-N,N-dimethylpicolinamide

The title compound was prepared in accordance with the synthetic protocols set forth in Example 84 Step 2 using appropriate intermediates, as well as commercial starting materials. LC-MS calc. for C₃₉H₄₄N₇O₅ [M+H]⁺: m/z=690.3; Found: 690.3.

### Example 169. 6-((8-(3-((5-Acetyl-2-(cyclopropanecarboxamido)pyridin-4-yl)amino)-2-methoxyphenyl)-1-(tert-butyl)-2-oxo-1,2,3,5-tetrahydro-4H-pyrido[3,2-e][1,4]diazepin-4-yl)methyl)-N,N-dimethylpicolinamide

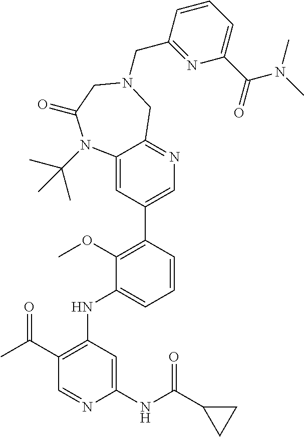 [SMILES: COc1c(Nc2cc(NC(=O)C3CC3)ncc2C(C)=O)cccc1-c1cnc2c(c1)N(C(C)(C)C)C(=O)CN(Cc1cccc(C(=O)N(C)C)n1)C2]

### Step 1. 5-Bromo-3-(tert-butylamino)picolinonitrile

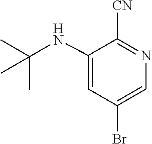

tert-Butylamine (780 μL, 7.5 mmol) was added to a stirring solution of N,N-diisopropylethylamine (1.7 mL, 9.9 mmol) and 5-bromo-3-fluoropicolinonitrile (1.0 g, 5.0 mmol) in DMF (10 mL). The reaction mixture was heated to 100° C. and stirred for 2 hours. The product mixture was diluted with EtOAc (100 mL) and washed with water (100 mL) and brine (2×100 mL). The organic layer was dried over Na₂SO₄, filtered and concentrated under reduced pressure. The residue obtained was purified by silica gel column chromatography (0-100% EtOAc/Hexane) to yield the title compound (1.13 g, 89% yield). LC-MS calc. for C₁₀H₁₃BrN₃ [M+H]⁺: m/z=254.0; Found: 254.0.

### Step 2. 2-(Aminomethyl)-5-bromo-N-(tert-butyl)pyridin-3-amine

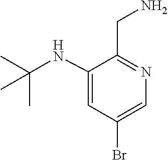

Borane-dimethyl sulfide (1.27 mL, 13.4 mmol) was added to a stirring solution of 5-bromo-3-(tert-butylamino)picolinonitrile (1.13 g, 4.46 mmol) in THF (22 mL). The reaction mixture was heated to 50° C. and stirred for 1 hour. The product mixture was cooled to 0° C. and quenched with dropwise addition of MeOH (3 mL). The product mixture was diluted with 1 N HCl (aq) (30 mL) and stirred at rt for 1 hour. The diluted product mixture was basified with sat. Na₂CO₃ (aq) (100 mL) and extracted with a 3:1 CHCl₃/iPrOH mixture (3×60 mL). The combined organic layers were dried over MgSO₄, filtered and concentrated under reduced pressure. The residue obtained was purified by silica gel column chromatography (0-20% MeOH/DCM) to yield the title compound (424 mg, 37% yield). LC-MS calc. for C₁₀H₁₇BrN₃ [M+H]⁺: m/z=258.1; Found: 258.0.

### Step 3. Methyl 6-((((5-bromo-3-(tert-butylamino)pyridin-2-yl)methyl)(tert-butoxycarbonyl)amino)methyl)picolinate

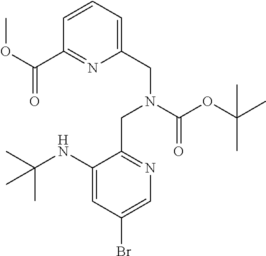 [SMILES: COC(=O)c1cccc(CN(Cc2ncc(Br)cc2NC(C)(C)C)C(=O)OC(C)(C)C)n1]

Methyl 6-formylpicolinate (217 mg, 1.31 mmol) was added to a stirring solution of 2-(aminomethyl)-5-bromo-N-(tert-butyl)pyridin-3-amine (424 mg, 1.6 mmol) in MeOH (8 mL). The reaction mixture was stirred overnight. Then sodium cyanoborohydride (206 mg, 3.3 mmol) was added and the reaction mixture was stirred another hour. The product mixture was diluted with sat. Na₂CO₃ (aq) (30 mL) and extracted with DCM (3×30 mL). The combined organic layers were dried over Na₂SO₄, filtered and concentrated under reduced pressure. The residue obtained was dissolved in THF (9 mL). Triethylamine (458 μL, 3.28 mmol) and Di-tert-butyl decarbonate (717 mg, 3.28 mmol) were added in sequence, and the reaction mixture was stirred at rt for 1 hour. The product mixture was concentrated under reduced pressure. The residue obtained was purified by silica gel column chromatography (0-100% EtOAc/Hexane) to yield the title compound (143 mg, 17% yield). LC-MS calc. for C₂₃H₃₂BrN₄O₄[M+H]⁺: m/z=507.2; Found: 507.1.

### Step 4. Methyl 6-((((5-bromo-3-(N-(tert-butyl)-2-chloroacetamido)pyridin-2-yl)methyl)(tert-butoxycarbonyl)amino)methyl)picolinate

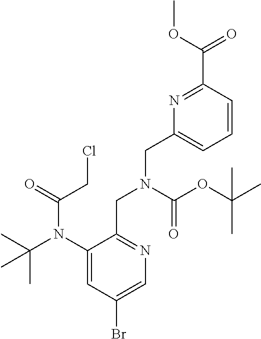 [SMILES: COC(=O)c1cccc(CN(Cc2ncc(Br)cc2N(C(=O)CCl)C(C)(C)C)C(=O)OC(C)(C)C)n1]

Chloroacetyl chloride (28 μL, 0.35 mmol) was added to a stirring solution of methyl 6-((((5-bromo-3-(tert-butylamino)pyridin-2-yl)methyl)(tert-butoxycarbonyl)amino)methyl)picolinate (60 mg, 0.12 mmol) and triethylamine (165 μL, 1.2 mmol) in 1,2-dichloroethane (1 mL). The reaction mixture was heated to 80° C. and stirred for 2 hours. The product mixture was diluted with water (30 mL) and extracted with DCM (3×30 mL). The combined organic layers were dried over MgSO₄, filtered and concentrated under reduced pressure. The residue obtained was purified by silica gel column chromatography (0-80% EtOAc/Hexane) to yield the title compound (30 mg, 43% yield). LC-MS calc. for C₂₅H₃₃BrClN₄O₅[M+H]⁺: m/z=583.1/585.1; Found: 583.1/585.1.

### Step 5. Methyl 6-((((5-bromo-3-(N-(tert-butyl)-2-chloroacetamido)pyridin-2-yl)methyl)amino)methyl)picolinate

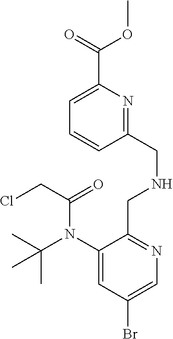 [SMILES: COC(=O)c1cccc(CNCc2ncc(Br)cc2N(C(=O)CCl)C(C)(C)C)n1]

Hydrochloric acid (0.72 mL, 2.9 mmol, 4.0 M in 1,4-dioxane) was added to a stirring solution of methyl 6-((((5-bromo-3-(N-(tert-butyl)-2-chloroacetamido)pyridin-2-yl)methyl)(tert-butoxycarbonyl)amino)methyl)picolinate (70 mg, 0.12 mmol) in DCM (700 μL). The reaction mixture was stirred at rt for 16 hours. The product mixture was concentrated under reduced pressure to obtain the HCl sat of the title compound (57 mg, 98% yield). LC-MS calc. for C₂₀H₂₅BrClN₄O₃ [M+H]⁺: m/z=483.1/485.1; Found: 483.1/485.1.

### Step 6. Methyl 6-((8-bromo-1-(tert-butyl)-2-oxo-1,2,3,5-tetrahydro-4H-pyrido[3,2-e][1,4]diazepin-4-yl)methyl)picolinate

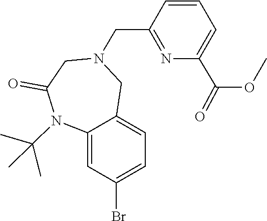 [SMILES: COC(=O)c1cccc(CN2CC(=O)N(C(C)(C)C)c3cc(Br)ccc3C2)n1]

N,N-Diisopropylethylamine (87 μL, 0.50 mmol) and sodium iodide (11 mg, 75 μmol) were added in sequence to a stirring solution of methyl 6-((((5-bromo-3-(N-(tert-butyl)-2-chloroacetamido)pyridin-2-yl)methyl)amino)methyl)picolinate hydrochloride (130 mg, 0.25 mmol) in MeCN (1.3 mL) at rt. The reaction mixture was stirred for 30 minutes. The product mixture was diluted with water (30 mL) and extracted with DCM (3×30 mL). The combined organic layers were dried over MgSO₄, filtered and concentrated under reduced pressure. The residue obtained was purified by silica gel column chromatography (0-5% MeOH/DCM) to yield the title compound (25 mg, 22% yield). LC-MS calc. for C₂₀H₂₄BrN₄O₃[M+H]⁺: m/z=447.1/449.1; Found: 447.0/449.0.

### Step 7. 6-((8-Bromo-1-(tert-butyl)-2-oxo-1,2,3,5-tetrahydro-4H-pyrido[3,2-e][1,4]diazepin-4-yl)methyl)picolinic acid

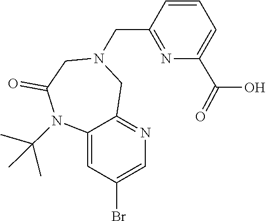 [SMILES: CC(C)(C)N1C(=O)CN(Cc2cccc(C(=O)O)n2)Cc2ncc(Br)cc21]

Lithium hydroxide (13 mg, 560 μmol) was added to a stirring solution of methyl 6-((8-bromo-1-(tert-butyl)-2-oxo-1,2,3,5-tetrahydro-4H-pyrido[3,2-e][1,4]diazepin-4-yl)methyl)picolinate (25 mg, 56 μmol) in THF (500 μL) and water (500 μL). The reaction mixture was stirred for 1 hour. The product mixture was acidified with 1 N HCl (aq) (30 mL) and extracted with a 3:1 CHCl₃/iPrOH mixture (3×30 mL). The combined organic layers were dried over MgSO₄, filtered and concentrated under reduced pressure to obtain the title compound (21 mg, 87% yield) which was used without further purification. LC-MS calc. for C₁₉H₂₂BrN₄O₃[M+H]⁺: m/z=433.1/435.1; Found: 433.0/435.0.

### Step 8. 6-((8-Bromo-1-(tert-butyl)-2-oxo-1,2,3,5-tetrahydro-4H-pyrido[3,2-e][1,4]diazepin-4-yl)methyl)-N,N-dimethylpicolinamide

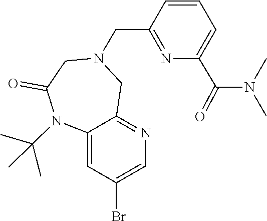 [SMILES: CN(C)C(=O)c1cccc(CN2CC(=O)N(C(C)(C)C)c3cc(Br)cnc3C2)n1]

1-[Bis(dimethylamino)methylene]-1H-1,2,3-triazolo[4,5-b]pyridinium 3-oxid hexafluorophosphate (33 mg, 88 μmol) was added to a stirring solution of N,N-diisopropylethylamine (31 μL, 220 μmol), dimethylamine hydrochloride (11 mg, 132 μmol) and 6-((8-bromo-1-(tert-butyl)-2-oxo-1,2,3,5-tetrahydro-4H-pyrido[3,2-e][1,4]diazepin-4-yl)methyl)picolinic acid (19 mg, 44 μmol) in DMF (1 mL) at 0° C. The reaction mixture was stirred for 30 minutes at 0° C. The product mixture was diluted with sat. NaHCO₃ (aq) (30 mL) and extracted with a 3:1 CHCl₃/iPrOH mixture (2×30 mL). The combined organic layers were dried over MgSO₄, filtered and concentrated under reduced pressure. The residue obtained was purified by silica gel column chromatography (0-5% MeOH/DCM) to yield the title compound (18 mg, 89% yield). LC-MS calc. for C₂₁H₂₇BrN₅O₂ [M+H]⁺: m/z=460.1/462.1; Found: 460.1/462.1.

### Step 9. 6-((8-(3-((5-Acetyl-2-(cyclopropanecarboxamido)pyridin-4-yl)amino)-2-methoxyphenyl)-1-(tert-butyl)-2-oxo-1,2,3,5-tetrahydro-4H-pyrido[3,2-e][1,4]diazepin-4-yl)methyl)-N,N-dimethylpicolinamide

The title compound was prepared in accordance with the synthetic protocols set forth in Example 84 Step 2 using appropriate intermediates, as well as commercial starting materials. LC-MS calc. for C₃₉H₄₅N₈O₅ [M+H]⁺: m/z=705.3; Found: 705.3.

### Example 170. (S)-6-((8-(3-((5-Acetyl-2-(cyclopropanecarboxamido)pyridin-4-yl)amino)-2-methoxyphenyl)-1-(1-acetylpyrrolidin-3-yl)-2-oxo-1,2,3,5-tetrahydro-4H-benzo[e][1,4]diazepin-4-yl)methyl)-N,N-dimethylpicolinamide

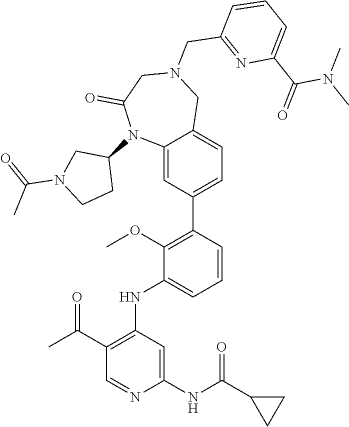 [SMILES: COc1c(Nc2cc(NC(=O)C3CC3)ncc2C(C)=O)cccc1-c1ccc2c(c1)N([C@H]1CCN(C(C)=O)C1)C(=O)CN(Cc1cccc(C(=O)N(C)C)n1)C2]

### Step 1. tert-Butyl (S)-3-((5-bromo-2-cyanophenyl)amino)pyrrolidine-1-carboxylate

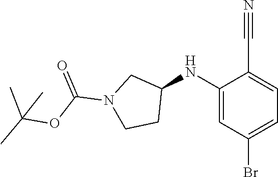 [SMILES: CC(C)(C)OC(=O)N1CC[C@H](Nc2cc(Br)ccc2C#N)C1]

N,N-Diisopropylethylamine (260 μL, 1.5 mmol) was added to a stirring solution of tert-butyl (S)-3-aminopyrrolidine-1-carboxylate (186 mg, 1.0 mmol) and 4-bromo-2-fluorobenzonitrile (100 mg, 0.5 mmol) in N-methyl-2-pyrrolidone (1 mL). The reaction mixture was heated to 120° C. for 4 hours. The product mixture was diluted with EtOAc (50 mL) and washed with sat. brine (aq) solution (3×50 mL). The organic layer was dried over Na₂SO₄, filtered and concentrated under reduced pressure. The residue obtained was purified by silica gel column chromatography (0-60% EtOAc/Hexanes) to yield the title compound (183 mg, 100% yield). LC-MS calc. for C₁₁H₁₃BrN₃ [M+H—C₅H₈O₂]⁺: m/z=266.1/268.1; Found: 266.0/268.0.

### Step 2. tert-Butyl (S)-3-((5-bromo-2-formylphenyl)amino)pyrrolidine-1-carboxylate

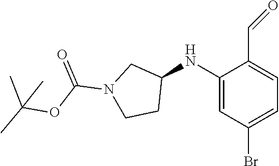 [SMILES: CC(C)(C)OC(=O)N1CC[C@H](Nc2cc(Br)ccc2C=O)C1]

Diisobutylaluminium hydride (1.15 mL, 1.38 mmol, 1.2 M in toluene) was added to a stirring solution of tert-butyl (S)-3-((5-bromo-2-cyanophenyl)amino)pyrrolidine-1-carboxylate (252 mg, 688 μmol) in DCM (3 mL) at −78° C. The reaction mixture was stirred for 1 hour at −78° C. The product mixture was quenched with MeOH (2 mL) at −78° C. and sat. Rochelle salt (aq) solution (5 mL). The quenched reaction mixture was stirred at rt for 30 minutes. The product mixture was diluted with sat. brine (aq) solution (50 mL) and extracted with EtOAc (3×50 mL).

The combined organic layers were dried over MgSO₄, filtered and concentrated under reduced pressure. The residue obtained was purified by silica gel column chromatography (0-80% EtOAc/Hexanes) to yield the title compound (159 mg, 63% yield). LC-MS calc. for C₁₂H₁₄BrN₂O₃ [M+H—C₄H₈]⁺: m/z=313.0/315.0; Found: 313.0/315.0.

### Step 3. tert-Butyl (S)-3-((5-bromo-2-(((2-methoxy-2-oxoethyl)amino)methyl)phenyl)-amino)pyrrolidine-1-carboxylate

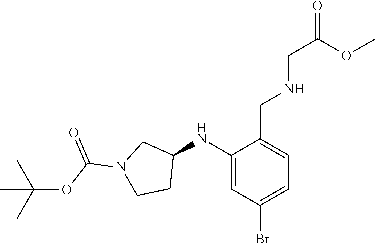 [SMILES: COC(=O)CNCc1ccc(Br)cc1N[C@H]1CCN(C(=O)OC(C)(C)C)C1]

Sodium cyanoborohydride (54 mg, 0.86 mmol) was added to a stirring solution of tert-butyl (S)-3-((5-bromo-2-formylphenyl)amino)pyrrolidine-1-carboxylate (159 mg, 0.43 mmol) and methyl glycinate hydrochloride (162 mg, 1.29 mmol) in MeOH (3 mL). The reaction mixture was stirred at rt for 16 hours. The product mixture was diluted with EtOAc (50 mL) and washed with sat. NaHCO₃ (aq) solution (50 mL) and sat. brine (aq) solution (50 mL). The organic layer was dried over Na₂SO₄, filtered and concentrated under reduced pressure. The residue obtained was purified by silica gel column chromatography (0-80% EtOAc/Hexanes) to yield the title compound (150 mg, 79% yield). LC-MS calc. for C₁₉H₂₉BrN₃O₄[M+H]⁺: m/z=442.1/444.1; Found: 442.1/444.1.

### Step 4. tert-Butyl (S)-3-((5-bromo-2-((((6-(dimethylcarbamoyl)pyridin-2-yl)methyl)(2-methoxy-2-oxoethyl)amino)methyl)phenyl)amino)pyrrolidine-1-carboxylate

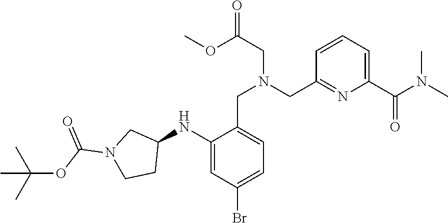 [SMILES: COC(=O)CN(Cc1cccc(C(=O)N(C)C)n1)Cc1ccc(Br)cc1N[C@H]1CCN(C(=O)OC(C)(C)C)C1]

Potassium carbonate (141 mg, 1.02 mmol) was added to a stirring solution of tert-butyl (S)-3-((5-bromo-2-(((2-methoxy-2-oxoethyl)amino)methyl)phenyl)amino)pyrrolidine-1-carboxylate (150 mg, 0.34 mmol) and 6-(bromomethyl)-N,N-dimethylpicolinamide (Int. 23) (95 mg, 0.39 mmol) in MeCN at rt. The reaction mixture was heated to 50° C. for 6 hours. The product mixture was diluted with sat. NaHCO₃ (aq) solution (20 mL) and sat. brine (aq) solution (20 mL) then extracted with a 3:1 CHCl₃/iPrOH mixture (3×40 mL). The combined organic layers were dried over MgSO₄, filtered and concentrated under reduced pressure. The residue obtained was purified by silica gel column chromatography (0-10% MeOH/DCM) to yield the title compound (205 mg, 100% yield). LC-MS calc. for C₂₈H₃₉BrN₅O₅[M+H]⁺: m/z=604.2/606.2; Found: 604.3/606.3.

### Step 5. (S)—N-(4-Bromo-2-((1-(tert-butoxycarbonyl)pyrrolidin-3-yl)amino)benzyl)-N-((6-(dimethylcarbamoyl)pyridin-2-yl)methyl)glycine

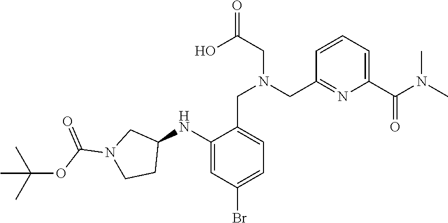 [SMILES: CN(C)C(=O)c1cccc(CN(CC(=O)O)Cc2ccc(Br)cc2N[C@H]2CCN(C(=O)OC(C)(C)C)C2)n1]

Lithium hydroxide (81 mg, 3.4 mmol) was added to a stirring solution of tert-butyl (S)-3-((5-bromo-2-((((6-(dimethylcarbamoyl)pyridin-2-yl)methyl)(2-methoxy-2-oxoethyl)amino)methyl)-phenyl)amino)pyrrolidine-1-carboxylate (205 mg, 0.34 mmol) in THF (2 mL) and water (2 mL) at rt. The reaction mixture was stirred for 30 minutes. The product mixture was acidified with 1 N HCl (aq) (30 mL) and extracted with a 3:1 CHCl₃/iPrOH mixture (3×40 mL). The combined organic layers were dried over MgSO₄, filtered and concentrated under reduced pressure to obtain the title compound (200 mg, 100% yield) which was used without further purification. LC-MS calc. for C₂₇H₃₇BrN₅O₅[M+H]⁺: m/z=590.2/592.2; Found: 590.3/592.3.

### Step 6. (S)-6-((1-(1-Acetylpyrrolidin-3-yl)-8-bromo-2-oxo-1,2,3,5-tetrahydro-4H-benzo[e][1,4]diazepin-4-yl)methyl)-N,N-dimethylpicolinamide

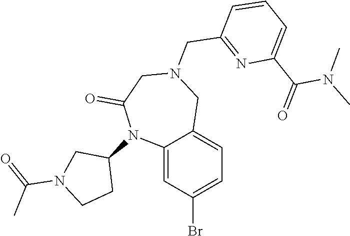 [SMILES: CC(=O)N1CC[C@H](N2C(=O)CN(Cc3cccc(C(=O)N(C)C)n3)Cc3ccc(Br)cc32)C1]

1-[Bis(dimethylamino)methylene]-1H-1,2,3-triazolo[4,5-b]pyridinium 3-oxid hexafluorophosphate (129 mg, 0.34 mmol) was added to a stirring solution of N,N-diisopropylethylamine (189 μL, 1.35 mmol) and (S)—N-(4-bromo-2-((1-(tert-butoxycarbonyl)pyrrolidin-3-yl)amino)benzyl)-N-((6-(dimethylcarbamoyl)pyridin-2-yl)methyl)glycine (19 mg, 44 μmol) in MeCN (6 mL) at 0° C. The reaction mixture was warmed to rt and stirred for 4 hours. The product mixture was diluted with sat. NaHCO₃ (aq) (30 mL) and extracted with a 3:1 CHCl₃/iPrOH mixture (2×30 mL). The combined organic layers were dried over MgSO₄, filtered and concentrated under reduced pressure. The residue obtained was purified by silica gel column chromatography (0-10% MeOH/DCM) to yield the title compound (67 mg, 35% yield). LC-MS calc. for C₂₁H₂₇BrN₅O₂[M+H]⁺: m/z=460.1/462.1; Found: 460.1/462.1.

### Step 7. tert-Butyl (S)-3-(8-(3-((5-acetyl-2-(cyclopropanecarboxamido)pyridin-4-yl)amino)-2-methoxyphenyl)-4-((6-(dimethylcarbamoyl)pyridin-2-yl)methyl)-2-oxo-2,3,4,5-tetrahydro-1H-benzo[e][1,4]diazepin-1-yl)pyrrolidine-1-carboxylate

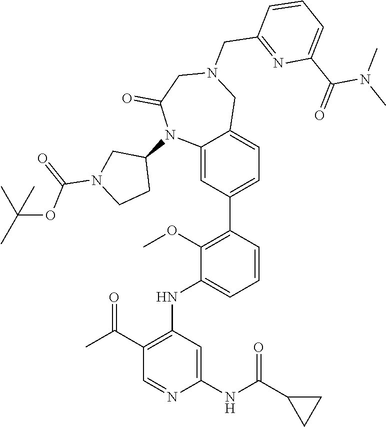 [SMILES: COc1c(Nc2cc(NC(=O)C3CC3)ncc2C(C)=O)cccc1-c1ccc2c(c1)N([C@H]1CCN(C(=O)OC(C)(C)C)C1)C(=O)CN(Cc1cccc(C(=O)N(C)C)n1)C2]

A 20 mL scintillation vial was charged with bis(pinacolato)diboron (54 mg, 0.21 mmol), potassium pivalate (49 mg, 0.35 mmol), [1,1′-bis(diphenylphosphino)ferrocene]dichloro-palladium(II) complex with dichloromethane (12 mg, 15 μmol), and (S)-6-((1-(1-acetylpyrrolidin-3-yl)-8-bromo-2-oxo-1,2,3,5-tetrahydro-4H-benzo[e][1,4]diazepin-4-yl)methyl)-N,N-dimethylpicolinamide (67 mg, 0.12 mmol). The mixture was dissolved in 1,4-dioxane (1.2 mL), was sparged with nitrogen gas for 5 minutes and sealed. Then the reaction mixture was heated to 95° C. for 50 minutes. The mixture was cooled to rt, and N-(5-acetyl-4-((3-bromo-2-methoxyphenyl)amino)-pyridin-2-yl)cyclopropanecarboxamide (Int. 3) (52 mg, 0.12 mmol), potassium carbonate (48 mg, 0.35 mmol), [1,1′-bis(diphenylphosphino)ferrocene]dichloropalladium (II) complex with dichloromethane (9.5 mg, 12 μmol), 1,4-dioxane (1 mL) and water (500 μL) were added to the reaction mixture sequentially. The mixture was sparged with nitrogen gas for 5 minutes and sealed. The reaction mixture was heated to 80° C. for 3 hours. The product mixture was diluted with sat. brine (aq) solution (40 mL) and extracted with a 3:1 CHCl₃/iPrOH mixture (2×30 mL). The combined organic layers were dried over MgSO₄, filtered and concentrated under reduced pressure. The residue obtained was purified by silica gel column chromatography (0-15% MeOH/DCM) to yield the title compound (30 mg, 32% yield). LC-MS calc. for C₄₅H₅₃N₈O₇ [M+H]⁺: m/z=817.4; Found: 817.6.

### Step 8. (S)-6-((8-(3-((5-Acetyl-2-(cyclopropanecarboxamido)pyridin-4-yl)amino)-2-methoxyphenyl)-2-oxo-1-(pyrrolidin-3-yl)-1,2,3,5-tetrahydro-4H-benzo[e][1,4]diazepin-4-yl)methyl)-N,N-dimethylpicolinamide

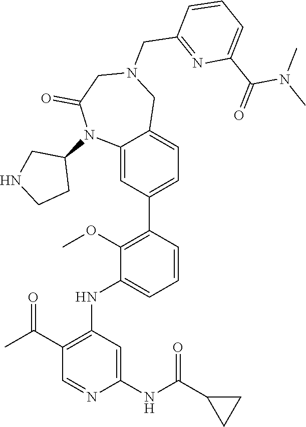 [SMILES: COc1c(Nc2cc(NC(=O)C3CC3)ncc2C(C)=O)cccc1-c1ccc2c(c1)N([C@H]1CCNC1)C(=O)CN(Cc1cccc(C(=O)N(C)C)n1)C2]

Trifluoroacetic acid (100 μL, 1.3 mmol) was added to a stirring solution of tert-butyl (S)-3-(8-(3-((5-acetyl-2-(cyclopropanecarboxamido)pyridin-4-yl)amino)-2-methoxyphenyl)-4-((6-(dimethylcarbamoyl)pyridin-2-yl)methyl)-2-oxo-2,3,4,5-tetrahydro-1H-benzo[e][1,4]diazepin-1-yl)pyrrolidine-1-carboxylate (31 mg, 38 μmol) in DCM (1 mL). The reaction mixture was stirred for 3 hours. The product mixture was concentrated under reduced pressure to obtain the TFA salt of the title compound (30 mg, 84% yield) which was used without further purification. LC-MS calc. for C₄₀H₄₅N₈O₅ [M+H]⁺: m/z=717.3; Found: 717.4.

### Step 9. (S)-6-((8-(3-((5-Acetyl-2-(cyclopropanecarboxamido)pyridin-4-yl)amino)-2-methoxyphenyl)-1-(I-acetylpyrrolidin-3-yl)-2-oxo-1,2,3,5-tetrahydro-4H-benzo[e][1,4]diazepin-4-yl)methyl)-N,N-dimethylpicolinamide

Acetyl chloride (2.2 μL, 31 μmol) was added to a stirring solution of the TFA salt of (S)-6-((8-(3-((5-acetyl-2-(cyclopropanecarboxamido)pyridin-4-yl)amino)-2-methoxyphenyl)-2-oxo-1-(pyrrolidin-3-yl)-1,2,3,5-tetrahydro-4H-benzo[e][1,4]diazepin-4-yl)methyl)-N,N-dimethylpicolinamide (22 mg, 21 μmol) and pyridine (17 μL, 0.21 mmol) in DCM (500 μL) at 0° C. The reaction mixture was stirred at 0° C. for 1 hour. The product mixture was diluted with MeOH (4 mL) and purified directly by prep-HPLC on a C₁₈ column (13.3-33.3% MeCN in 0.2% TFA(aq)) to afford the TFA salt of the title compound (5.3 mg, 26% yield). LC-MS calc. for C₄₂H₄₆N₈O₆ [M+H]⁺: m/z=759.4; Found: 759.4.

### Example 171. 6-((8-(3-((5-Acetyl-2-(cyclopropanecarboxamido)pyridin-4-yl)amino)-2-methoxyphenyl)-1-(azetidin-3-yl)-2-oxo-1,2,3,5-tetrahydro-4H-benzo[e][1,4]diazepin-4-yl)methyl)-N,N-dimethylpicolinamide

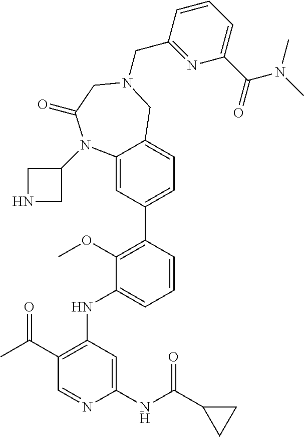 [SMILES: COc1c(Nc2cc(NC(=O)C3CC3)ncc2C(C)=O)cccc1-c1ccc2c(c1)N(C1CNC1)C(=O)CN(Cc1cccc(C(=O)N(C)C)n1)C2]

### Step 1. tert-Butyl 3-(8-(3-((5-acetyl-2-(cyclopropanecarboxamido)pyridin-4-yl)amino)-2-methoxyphenyl)-4-((6-(dimethylcarbamoyl)pyridin-2-yl)methyl)-2-oxo-2,3,4,5-tetrahydro-1H-benzo[e][1,4]diazepin-1-yl)azetidine-1-carboxylate

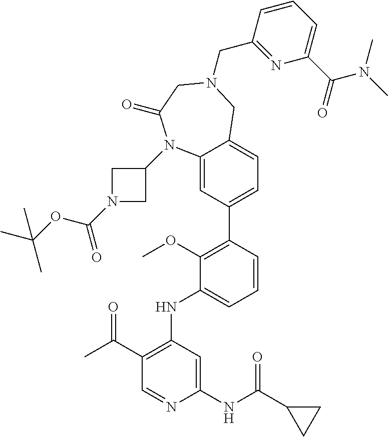 [SMILES: COc1c(Nc2cc(NC(=O)C3CC3)ncc2C(C)=O)cccc1-c1ccc2c(c1)N(C1CN(C(=O)OC(C)(C)C)C1)C(=O)CN(Cc1cccc(C(=O)N(C)C)n1)C2]

The title compound was prepared in accordance with the synthetic protocols set forth in Example 170 Steps 1 through Step 7 using appropriate intermediates, as well as commercial starting materials. LC-MS calc. for C₄₄H₅₁N₈O₇ [M+H]⁺: m/z=803.4; Found: 803.5.

### Step 2. 6-((8-(3-((5-Acetyl-2-(cyclopropanecarboxamido)pyridin-4-yl)amino)-2-methoxyphenyl)-1-(azetidin-3-yl)-2-oxo-1,2,3,5-tetrahydro-4H-benzo[e][1,4]diazepin-4-yl)methyl)-N,N-dimethylpicolinamide

Trifluoroacetic acid (100 μL, 1.3 mmol) was added to a stirring solution of tert-butyl 3-(8-(3-((5-acetyl-2-(cyclopropanecarboxamido)pyridin-4-yl)amino)-2-methoxyphenyl)-4-((6-(dimethylcarbamoyl)pyridin-2-yl)methyl)-2-oxo-2,3,4,5-tetrahydro-1H-benzo[e][1,4]diazepin-1-yl)azetidine-1-carboxylate (40 mg, 50 μmol) in DCM (1 mL). The reaction mixture was stirred for 3 hours. The product mixture was concentrated under reduced pressure and purified by prep-HPLC on a C₁₈ column (10.9-30.9% MeCN in 0.2% TFA(aq)) to afford the TFA salt of the title compound (5.3 mg, 26% yield). LC-MS calc. for C₃₉H₄₃N₈O₅ [M+H]⁺: m/z=703.3; Found: 703.4.

### Example 172. 6-((8-(4-((5-Acetyl-2-(cyclopropanecarboxamido)pyridin-4-yl)amino)-3-methoxy-6-(trifluoromethyl)pyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide

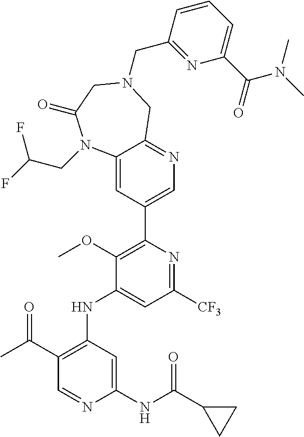 [SMILES: COc1c(Nc2cc(NC(=O)C3CC3)ncc2C(C)=O)cc(C(F)(F)F)nc1-c1cnc2c(c1)N(CC(F)F)C(=O)CN(Cc1cccc(C(=O)N(C)C)n1)C2]

### Step 1. 2-Chloro-4-iodo-6-(trifluoromethyl)pyridin-3-ol

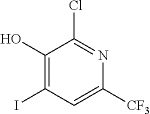 [SMILES: Oc1c(I)cc(C(F)(F)F)nc1Cl]

Iodine (1.93 g, 7.59 mmol) was added slowly to a stirring solution of 2-chloro-6-(trifluoromethyl)pyridin-3-ol (1.0 g, 5.1 mmol) and sodium hydroxide (607 mg, 15.2 mmol) in water (15 mL) at 0° C. The reaction mixture was stirred for 20 minutes at rt. Then an additional portion of iodine (1.93 g, 7.59 mmol) was added, and the reaction was heated to 80° C. for 4 hours. The product mixture was diluted with 1 N HCl (aq) solution (100 mL) and extracted with a 3:1 CHCl₃/iPrOH mixture (3×100 mL). The combined organic layers were dried over MgSO₄, filtered and concentrated under reduced pressure. The residue obtained was purified by silica gel column chromatography (0-5% MeOH/DCM) to yield the title compound (1.6 g, 60% yield). LC-MS calc. for C₆H₃ClF₃INO [M+H]⁺: m/z=323.9; Found: 324.1.

### Step 2. 2-Chloro-4-iodo-3-methoxy-6-(trifluoromethyl)pyridine

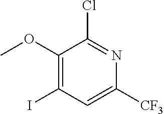

Potassium carbonate (990 mg, 7.2 mmol) and iodomethane (446 μL, 7.2 mmol) were added in sequence to a stirring solution of 2-chloro-4-iodo-6-(trifluoromethyl)pyridin-3-ol (773 mg, 2.4 mmol) in MeCN (12 mL) at rt. The reaction mixture was heated to 50° C. for 16 hours. The product mixture was diluted with water (60 mL) and extracted with EA (2×50 mL). The combined organic layers were washed with brine (50 mL), dried over Na₂SO₄, filtered, and concentrated under reduced pressure. The residue obtained was purified by silica gel column chromatography (0-70% EtOAc/Hexanes) to yield the title compound (800 mg, 99% yield). LC-MS calc. for C₇H₅ClF₃INO [M+H]⁺: m/z=337.9; Found: 338.2.

### Step 3. tert-Butyl (2-chloro-3-methoxy-6-(trifluoromethyl)pyridin-4-yl)carbamate

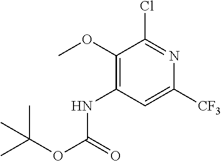 [SMILES: COc1c(NC(=O)OC(C)(C)C)cc(C(F)(F)F)nc1Cl]

A 20 mL scintillation vial was charged with 2-chloro-4-iodo-3-methoxy-6-(trifluoro-methyl)pyridine (83 mg, 0.25 mmol), tert-butyl carbamate (43 mg, 0.37 mmol), tris(dibenzyl-ideneacetone)dipalladium (0) (23 mg, 25 μmol), 4,5-bis(diphenylphosphino)-9,9-dimethylxanthene (29 mg, 49 μmol), and cesium carbonate (241 mg, 0.74 mmol). The reaction mixture was dissolved in toluene (2.5 mL), sparged with nitrogen gas for 5 minutes, and sealed. Then the reaction mixture was heated to 100° C. for 2 hours. The product mixture was diluted with water (30 mL) and extracted with EtOAc (3×30 mL). The combined organic layers were dried over Na₂SO₄, filtered and concentrated under reduced pressure. The residue obtained was purified by silica gel column chromatography (0-50% EtOAc/Hexane) to yield the title compound (79 mg, 98% yield). LC-MS calc. for C₈H₇C₁F₃N₂O₃ [M+H—C₄H₈]⁺: m/z=271.0; Found: 271.3.

### Step 4. 2-Chloro-3-methoxy-6-(trifluoromethyl)pyridin-4-amine

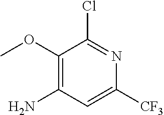

Trifluoroacetic acid (300 μL, 3.9 mmol) was added to a stirring solution of tert-butyl (2-chloro-3-methoxy-6-(trifluoromethyl)pyridin-4-yl)carbamate (79 mg, 0.24 mmol) in DCM (700 μL). The reaction mixture was stirred at rt for 1 hour. The product mixture was diluted with sat. NaHCO₃ (aq) solution (30 mL) and extracted with DCM (3×30 mL). The combined organic layers were dried over Na₂SO₄, filtered and concentrated under reduced pressure. The residue obtained was purified by silica gel column chromatography (0-80% EtOAc/Hexane) to yield the title compound (30 mg, 54% yield). LC-MS calc. for C₇H₇ClF₃N₂O [M+H]⁺: m/z=227.0; Found: 227.2.

### Step 5. (1-(2,2-Difluoroethyl)-4-((6-(dimethylcarbamoyl)pyridin-2-yl)methyl)-2-oxo-2,3,4,5-tetrahydro-1H-pyrido[3,2-e][1,4]diazepin-8-yl)boronic acid

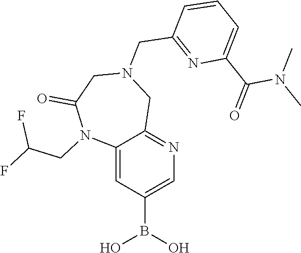 [SMILES: CN(C)C(=O)c1cccc(CN2CC(=O)N(CC(F)F)c3cc(B(O)O)cnc3C2)n1]

A 20 mL scintillation vial was charged with bis(pinacolato)diboron (312 mg, 1.23 mmol), potassium pivalate (345 mg, 2.46 mmol), [1,1′-bis(diphenylphosphino)ferrocene]dichloro-palladium(II) complex with dichloromethane (72 mg, 0.098 mmol), and 6-((8-bromo-1-(2,2-difluoroethyl)-2-oxo-1,2,3,5-tetrahydro-4H-pyrido[3,2-e][1,4]diazepin-4-yl)methyl)-N,N-dimethylpicolinamide (384 mg, 0.82 mmol). The mixture was dissolved in 1,4-dioxane (10 mL), was sparged with nitrogen gas for 5 minutes and sealed. Then the reaction mixture was heated to 90° C. for 1 hour. The product mixture was diluted with water (50 mL) and extracted with a 3:1 CHCl₃/iPrOH mixture (3×50 mL). The combined organic layers were dried over MgSO₄, filtered and concentrated under reduced pressure to yield the title compound (355 mg, 100% yield) which was used without further purification. LC-MS calc. for C₁₉H₂₃BF₂N₅O₄ [M+H]⁺: m/z=434.2; Found: 434.3.

### Step 6. 6-((8-(4-Amino-3-methoxy-6-(trifluoromethyl)pyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide

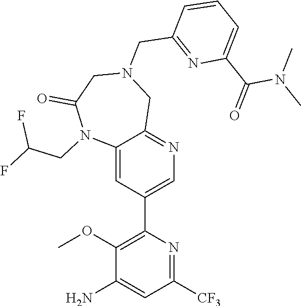 [SMILES: COc1c(N)cc(C(F)(F)F)nc1-c1cnc2c(c1)N(CC(F)F)C(=O)CN(Cc1cccc(C(=O)N(C)C)n1)C2]

A 20 mL scintillation vial was charged with (1-(2,2-difluoroethyl)-4-((6-(dimethyl-carbamoyl)pyridin-2-yl)methyl)-2-oxo-2,3,4,5-tetrahydro-1H-pyrido[3,2-e][1,4]diazepin-8-yl)boronic acid (68 mg, 0.16 mmol), 2-chloro-3-methoxy-6-methylpyridin-4-amine (81 mg, 0.19 mmol), potassium carbonate (54 mg, 0.39 mmol) and [1,1′-bis(di-tert-butylphosphino)ferrocene]dichloropalladium(II) (13 mg, 19 μmol). The mixture was dissolved in 1,4-dioxane (900 μL) and water (100 μL), sparged with nitrogen gas for 5 minutes and sealed. The reaction mixture was heated to 95° C. and was stirred for 4 hours. The product mixture was diluted with water (50 mL) and extracted with a 3:1 CHCl₃/iPrOH mixture (3×40 mL). The combined organic layers were dried over MgSO₄, filtered and concentrated under reduced pressure. The residue obtained was purified by prep-HPLC on a C₁₈ column (5-55% MeCN in 0.05% TFA(aq)) to afford the TFA salt of the title compound (34 mg, 44% yield). LCMS calc. for C₂₆H₂₇F₅N₇O₃ [M+H]⁺: m/z=580.2; Found: 580.5.

### Step 7. 6-((8-(4-((5-Acetyl-2-(cyclopropanecarboxamido)pyridin-4-yl)amino)-3-methoxy-6-(trifluoromethyl)pyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide

A 20 mL scintillation vial was charged with 6-((8-(4-amino-3-methoxy-6-(trifluoro-methyl)pyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide (34 mg, 58 μmmol), N-(5-acetyl-4-chloropyridin-2-yl)cyclopropanecarboxamide (Int. 18) (17 mg, 69 μmmol), palladium (II) acetate (1.9 mg, 8.7 μmol), 1,1′-bis(diphenylphosphino)ferrocene (29 mg, μmol) and cesium carbonate (56 mg, 0.17 mmol). The mixture was dissolved in 1,4-dioxane (1 mL) and water (1 μL), sparged with nitrogen gas for 5 minutes and sealed. The reaction mixture was heated to 100° C. for 1 hour. The product mixture was diluted with water (30 mL) and extracted with a 3:1 CHCl₃/iPrOH mixture (3×30 mL). The combined organic layers were dried over MgSO₄, filtered and concentrated under reduced pressure. The residue obtained was purified by prep-HPLC on a C₁₈ column (10-60% MeCN in 0.05% TFA(aq)) to afford the TFA salt of the title compound (45 mg, 100% yield). LCMS calc. for C₃₇H₃₇F₅N₉O₅[M+H]⁺: m/z=782.3; Found: 782.6.

### Examples 173-174

Examples 173-174 are shown below in Table 17 and were prepared in accordance with the synthetic protocols set forth in Example 172 using the appropriate intermediates, as well as commercial starting materials. The following compounds are TFA salts unless otherwise noted.

TABLE 17
Examples 173-174

| | | | Analytical Data (LCMS |
|---|---|---|---|
| Ex. | Name | Structure | and/or NMR) |
| 173 | (S)-6-((8-(4-((5-acetyl- 2-(cyclopropane- carboxamido)pyridin- 4-yl)amino)-3- methoxy-6- (trifluoromethyl) pyridin-2-yl)-2- oxo-1- (tetrahydrofuran-3-yl)- 1,2,3,5-tetrahydro-4H- pyrido[3,2-e ][1,4] diazepin-4- yl)methyl)-N,N- dimethylpicolinamide | 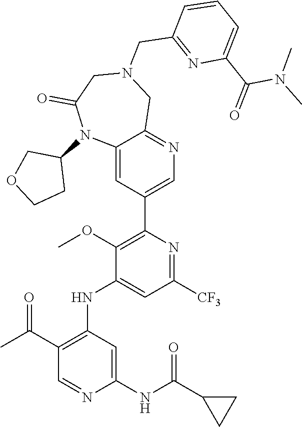 [SMILES: COc1c(Nc2cc(NC(=O)C3CC3)ncc2C(C)=O)cc(C(F)(F)F)nc1-c1cnc2c(c1)N([C@H]1CCOC1)C(=O)CN(Cc1cccc(C(=O)N(C)C)n1)C2] | LC-MS calc. for C₃₉H₄₁F₃N₉O₆ [M + H]⁺: m/z= 788.3; Found: 788.6 ¹H NMR (300 MHz, CDCl₃) δ 12.93 (s, 1H), 12.07 (s, 1H), 9.27 (d, J = 1.3 Hz, 1H), 8.76 (s, 1H), 8.69 (s, 1H), 8.53 (d, J = 1.3 Hz, 1H), 8.00- 7.88 (m, 2H), 7.63 (t, J = 7.5 Hz, 2H), 5.27 (d, J = 3.7 Hz, 1H), 4.54-4.32 (m, 4H), 4.26 (td, J = 8.8, 4.8 Hz, 1H), 4.05-3.90 (m, 2H), 3.85-3.65 (m, 5H), 3.11 (d, J = 25.6 Hz, 5H), 2.71 (d, J = 9.5 Hz, 3H), 2.37 (dd, J = 16.7, 12.1 Hz, 1H), 2.09-1.90 (m, 2H), 1.27 (d, J = 2.9 Hz, 2H), 1.20- 1.10 (m, 2H), 1.05 (dd, J = 7.6, 3.1 Hz, 2H). |
| 174 | 6-((8-(4-((5-acetyl-2- ((1S,2S)-2- methylcyclopropane- 1-carboxamido)- pyridin-4-yl)amino)-3- methoxy-6- (trifluoromethyl) pyridin-2-yl)-2- oxo-1-((S)- tetrahydrofuran-3-yl)- 1,2,3,5-tetrahydro-4H- pyrido[3,2-e ][1,4] diazepin-4- yl)methyl)-N,N- dimethylpicolinamide | 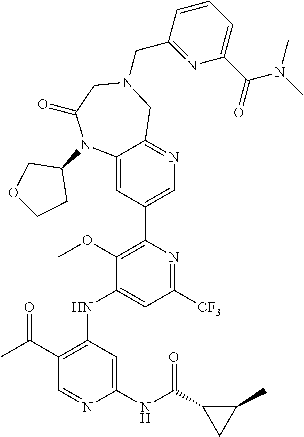 [SMILES: COc1c(Nc2cc(NC(=O)[C@H]3C[C@@H]3C)ncc2C(C)=O)cc(C(F)(F)F)nc1-c1cnc2c(c1)N([C@H]1CCOC1)C(=O)CN(Cc1cccc(C(=O)N(C)C)n1)C2] | LC-MS calc. for C₄₀H₄₃F₃N₉O₆ [M + H]⁺: m/z = 802.3; Found: 802.6 |

### Example 175. (S)-6-((8-(2-((5-Acetyl-2-(cyclopropanecarboxamido)pyridin-4-yl)amino)-3-methoxy-6-(trifluoromethyl)pyridin-4-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide

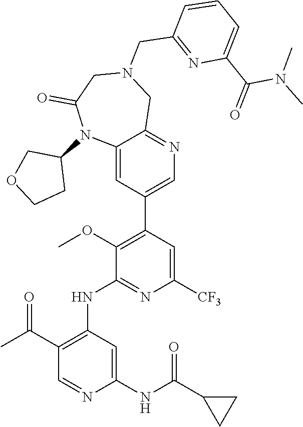 [SMILES: COc1c(-c2cnc3c(c2)N([C@H]2CCOC2)C(=O)CN(Cc2cccc(C(=O)N(C)C)n2)C3)cc(C(F)(F)F)nc1Nc1cc(NC(=O)C2CC2)ncc1C(C)=O]

### Step 1. (S)-(4-((6-(Dimethylcarbamoyl)pyridin-2-yl)methyl)-2-oxo-1-(tetrahydrofuran-3-yl)-2,3,4,5-tetrahydro-1H-pyrido[3,2-e][1,4]diazepin-8-yl)boronic acid

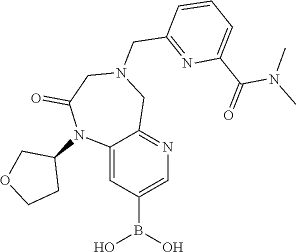 [SMILES: CN(C)C(=O)c1cccc(CN2CC(=O)N([C@H]3CCOC3)c3cc(B(O)O)cnc3C2)n1]

The title compound was prepared in accordance with the synthetic protocols set forth in Example 172 Step 5 using appropriate intermediates, as well as commercial starting materials. LC-MS calc. for C₂₁H₂₇BN₅O₅[M+H]⁺: m/z=440.2; Found: 440.4.

### Step 2. (S)-6-((8-(2-Chloro-3-methoxy-6-(trifluoromethyl)pyridin-4-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide

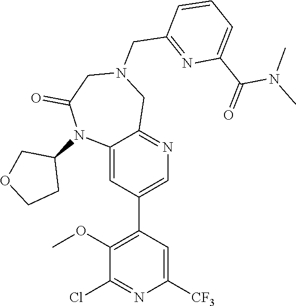 [SMILES: COc1c(-c2cnc3c(c2)N([C@H]2CCOC2)C(=O)CN(Cc2cccc(C(=O)N(C)C)n2)C3)cc(C(F)(F)F)nc1Cl]

A 20 mL scintillation vial was charged with (S)-(4-((6-(dimethylcarbamoyl)pyridin-2-yl)methyl)-2-oxo-1-(tetrahydrofuran-3-yl)-2,3,4,5-tetrahydro-1H-pyrido[3,2-e][1,4]diazepin-8-yl)boronic acid (137 mg, 0.31 mmol), 2-chloro-4-iodo-3-methoxy-6-(trifluoromethyl)pyridine (158 mg, 0.47 mmol), potassium carbonate (129 mg, 0.94 mmol) and [1,1′-bis(diphenylphosphino)-ferrocene]dichloropalladium (II) (34 mg, 47 μmol). The mixture was dissolved in 1,4-dioxane (2.7 mL) and water (300 μL), sparged with nitrogen gas for 5 minutes and sealed. The reaction mixture was heated to 70° C. for 2 hours. The product mixture was diluted with water (50 mL) and extracted with a 3:1 CHCl₃/iPrOH mixture (3×40 mL). The combined organic layers were dried over MgSO₄, filtered and concentrated under reduced pressure. The residue obtained was purified by silica gel column chromatography (0-30% MeOH/DCM) to yield the title compound (128 mg, 68% yield). LC-MS calc. for C₂₈H₂₉C₁F₃N₆O₄ [M+H]⁺: m/z=605.2; Found: 605.3.

### Step 3. (S)-6-((8-(2-((5-Acetyl-2-(cyclopropanecarboxamido)pyridin-4-yl)amino)-3-methoxy-6-(trifluoromethyl)pyridin-4-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide

A 20 mL scintillation vial was charged with (S)-6-((8-(2-chloro-3-methoxy-6-(trifluoromethyl)pyridin-4-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide (90 mg, 0.15 mmol), N-(5-acetyl-4-aminopyridin-2-yl)cyclopropanecarboxamide (49 mg, 0.22 mmol), BrettPhos Pd G3 (27 mg, 30 μmol), BrettPhos (16 mg, 30 μmol), and cesium carbonate (97 g, 0.30 mmol). The reaction mixture was dissolved in 1,4-dioxane (1.5 mL), sparged with nitrogen gas for 5 minutes, and sealed. Then the reaction mixture was heated to 100° C. for 2 hours. The product mixture was diluted with water (30 mL) and extracted with a 3:1 CHCl₃/iPrOH mixture (3×30 mL). The combined organic layers were dried over MgSO₄, filtered and concentrated under reduced pressure. The residue obtained was purified by prep-HPLC on a C₁₈ column (15-55% MeCN in 0.05% TFA(aq)) to afford the TFA salt of the title compound (29 mg, 25% yield). LC-MS calc. for C₃₉H₄₁F₃N₉O₆[M+H]⁺: m/z=788.3; Found: 788.6.

### Example 176. 6-((8-(4-((5-Acetyl-2-(cyclopropanecarboxamido)pyridin-4-yl)amino)-3-methoxypyridin-2-yl)-1-(bicyclo[1.1.1]pentan-1-yl)-2-oxo-1,2,3,5-tetrahydro-4H-benzo[e][1,4]diazepin-4-yl)methyl)-N,N-dimethylpicolinamide

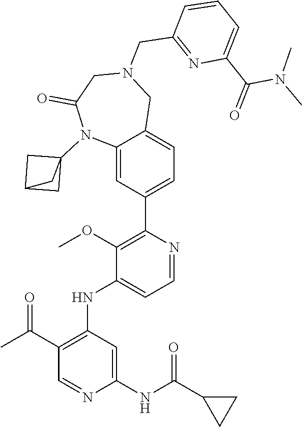 [SMILES: COc1c(Nc2cc(NC(=O)C3CC3)ncc2C(C)=O)ccnc1-c1ccc2c(c1)N(C13CC(C1)C3)C(=O)CN(Cc1cccc(C(=O)N(C)C)n1)C2]

### Step 1. tert-Butyl (4-bromo-2-iodobenzyl)glycinate

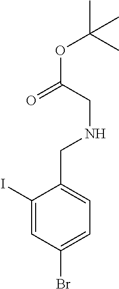 [SMILES: CC(C)(C)OC(=O)CNCc1ccc(Br)cc1I]

Sodium triacetoxyborohydride (2.04 g, 9.65 mmol) was added to a stirring solution of 4-bromo-2-iodobenzaldehyde (2.50 g, 8.04 mmol) and tert-butyl glycinate (1.27 g, 9.65 mmol) in DCM (20 mL). The reaction mixture was stirred at rt for 30 minutes. The product mixture was diluted with water (100 mL) and extracted with EtOAc (3×100 mL). The combined organic layers were dried over Na₂SO₄, filtered and concentrated under reduced pressure. The residue obtained was purified by silica gel column chromatography (0-20% EtOAc/Hexane) to yield the title compound (2.0 g, 58% yield). LC-MS calc. for C₁₃H₁₈BrINO₂ [M+H]⁺: m/z=426.0/428.0; Found: 425.9/427.9.

### Step 2. tert-Butyl N-(4-bromo-2-iodobenzyl)-N-(tert-butoxycarbonyl)glycinate

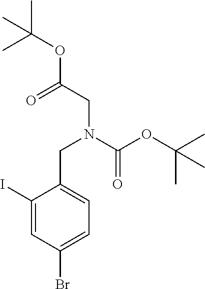 [SMILES: CC(C)(C)OC(=O)CN(Cc1ccc(Br)cc1I)C(=O)OC(C)(C)C]

Di-tert-butyl dicarbonate (1.42 mL, 6.2 mmol) was added to a stirring solution of tert-butyl (4-bromo-2-(cyclohexylamino)benzyl)glycinate (2.40 g, 5.63 mmol) and triethylamine (1.18 mL, 8.45 mmol) in DCM (48 mL) at rt. The reaction mixture was stirred for 16 hours. The product mixture was diluted with 1.0 N HCl (aq) (50 mL) and extracted with DCM (3×50 mL). The combined organic layers were dried over Na₂SO₄, filtered and concentrated under reduced pressure to yield the title compound (2.96 g, quant. yield) which was used without further purification. LCMS calc. for C₁₈H₂₆BrINO₄ [M+H]⁺: m/z=526.0/528.0; Found: 525.9/527.9.

### Step 3. N-(4-Bromo-2-iodobenzyl)-N-(tert-butoxycarbonyl)glycine

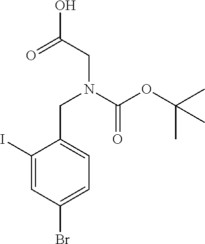 [SMILES: CC(C)(C)OC(=O)N(CC(=O)O)Cc1ccc(Br)cc1I]

Sodium hydroxide (1.09 g, 27.4 mmol) was added to a stirring solution of tert-butyl N-(4-bromo-2-iodobenzyl)-N-(tert-butoxycarbonyl)glycinate (2.4 g, 4.56 mmol) in MeOH (8 mL) and water (8 mL). The reaction mixture was heated to 60° C. and stirred for 2 hours. The product mixture was cooled to 0° C. and quenched with 1 N HCl (aq) solution to reach a pH of 3. The diluted product mixture was extracted with EtOAc (3×100 mL). The combined organic layers were dried over Na₂SO₄, filtered and concentrated under reduced pressure. The residue obtained was purified by silica gel column chromatography (0-30% EtOAc/Hexane) to yield the title compound (2.00 g, 76% yield). LC-MS calc. for C₁₄H₁₈BrINO₄ [M+H]⁺: m/z=470.0/472.0; Found: 469.9/471.9.

### Step 4. tert-Butyl (2-(bicyclo[1.1.1]pentan-1-ylamino)-2-oxoethyl)(4-bromo-2-iodobenzyl)carbamate

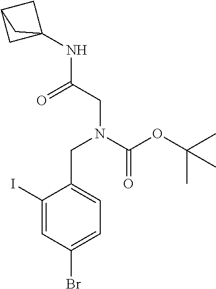 [SMILES: CC(C)(C)OC(=O)N(CC(=O)NC12CC(C1)C2)Cc1ccc(Br)cc1I]

1-[Bis(dimethylamino)methylene]-1H-1,2,3-triazolo[4,5-b]pyridinium 3-oxid hexafluorophosphate (300 mg, 0.789 mmol) was added to a stirring solution of triethylamine (275 μL, 1.97 mmol), bicyclo[1.1.1]pentan-1-amine hydrochloride (94 mg, 0.789 mmol) and N-(4-bromo-2-iodobenzyl)-N-(tert-butoxycarbonyl)glycine (309 mg, 0.658 mmol) in DCM (2 mL) at rt. The reaction mixture was stirred for 16 hours. The product mixture was diluted with water (50 mL) and extracted with EtOAc (3×50 mL). The combined organic layers were dried over Na₂SO₄, filtered, and concentrated under reduced pressure. The residue obtained was purified by silica gel column chromatography (0-25% EtOAc/Hexanes) to yield the title compound (279 mg, 79% yield). LC-MS calc. for C₁₉H₂₅BrIN₂O₃ [M+H]⁺: m/z=535.0/537.0; Found: 534.9/536.9.

### Step 5. tert-Butyl 1-(bicyclo[1.1.1]pentan-1-yl)-8-bromo-2-oxo-1,2,3,5-tetrahydro-4H-benzo[e][1,4]diazepine-4-carboxylate

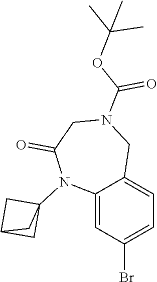 [SMILES: CC(C)(C)OC(=O)N1CC(=O)N(C23CC(C2)C3)c2cc(Br)ccc2C1]

A 20 mL scintillation vial was charged with tert-butyl (2-(bicyclo[1.1.1]pentan-1-ylamino)-2-oxoethyl)(4-bromo-2-iodobenzyl)carbamate (167 mg, 0.31 mmol), 3,4,7,8-Tetramethyl-1,10-phenanthroline (15 mg, 62 μmol), copper (II) acetate (11 mg, 62 μmol) and potassium phosphate tribasic (199 mg, 0.936 mmol). The mixture was dissolved in DMSO (1.5 mL), sparged with nitrogen gas for 5 minutes, and sealed. The reaction mixture was heated to 100° C. for 2 hours. The product mixture was diluted with water (30 mL) and extracted with EtOAc (3×50 mL). The combined organic layers were dried over MgSO₄, filtered and concentrated under reduced pressure. The residue obtained was purified by silica gel column chromatography (0-15% EtOAc/Heptane) to yield the title compound (73 mg, 57% yield). LC-MS calc. for C₁₉H₂₄BrN₂O₃[M+H]⁺: m/z=407.1/409.1; Found: 407.2/409.2.

### Step 6. 6-((1-(Bicyclo[1.1.1]pentan-1-yl)-8-bromo-2-oxo-1,2,3,5-tetrahydro-4H-benzo[e][1,4]diazepin-4-yl)methyl)-N,N-dimethylpicolinamide

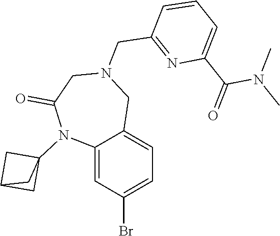 [SMILES: CN(C)C(=O)c1cccc(CN2CC(=O)N(C34CC(C3)C4)c3cc(Br)ccc3C2)n1]

Trifluoroacetic acid (500 μL, 6.5 mmol) was added to a stirring solution of tert-butyl 1-(bicyclo[1.1.1]pentan-1-yl)-8-bromo-2-oxo-1,2,3,5-tetrahydro-4H-benzo[e][1,4]diazepine-4-carboxylate (73 mg, 0.18 mmol) in DCM (1 mL). The reaction was stirred for 1 hour then concentrated under reduced vacuum. The residue was dissolved in DCM (1 mL) and 6-formyl-N,N-dimethylpicolinamide (38 mg, 0.22 mmol) was added. The reaction mixture was stirred for 10 minutes then sodium triacetoxyborohydride (38 mg, 0.18 mmol) was added. The reaction mixture was stirred for 16 hours at rt. The product mixture was purified directly by silica gel column chromatography (0-10% MeOH/DCM) to yield the title compound (50 mg, 59% yield). LC-MS calc. for C₂₃H₂₆BrN₄O₂[M+H]⁺: m/z=469.1/471.1; Found: 469.1/471.1.

### Step 7. 6-((1-(Bicyclo[1.1.1]pentan-1-yl)-2-oxo-8-(4,4,5,5-tetramethyl-1, 3,2-dioxaborolan-2-yl)-1,2,3,5-tetrahydro-4H-benzo[e][1,4]diazepin-4-yl)methyl)-N,N-dimethylpicolinamide

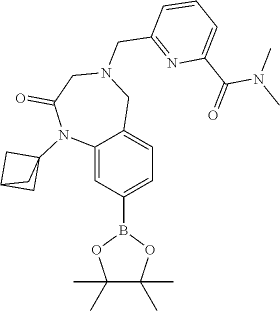 [SMILES: CN(C)C(=O)c1cccc(CN2CC(=O)N(C34CC(C3)C4)c3cc(B4OC(C)(C)C(C)(C)O4)ccc3C2)n1]

A 20 mL scintillation vial was charged with bis(pinacolato)diboron (49 mg, 0.19 mmol), potassium acetate (31 mg, 0.32 mmol), [1,1′-bis(diphenylphosphino)ferrocene]dichloropalladium(II) complex with dichloromethane (8.7 mg, 11 μmol), and 6-((1-(bicyclo[1.1.1]pentan-1-yl)-8-bromo-2-oxo-1,2,3,5-tetrahydro-4H-benzo[e][1,4]diazepin-4-yl)methyl)-N,N-dimethylpicolinamide (50 mg, 0.11 mmol). The mixture was dissolved in 1,4-dioxane (2 mL), was sparged with nitrogen gas for 5 minutes and sealed. Then the reaction mixture was heated to 90° C. for 1 hour. The product mixture was diluted with water (30 mL) and extracted with EtOAc (3×30 mL). The combined organic layers were dried over MgSO₄, filtered and concentrated under reduced pressure. The residue obtained was purified by silica gel column chromatography (0-2% MeOH/DCM) to yield the title compound (48 mg, 87% yield). LC-MS calc. for C₂₉H₃₈BN₄O₄[M+H]⁺: m/z=517.3; Found: 517.3.

### Step 8. 6-((8-(4-((5-Acetyl-2-(cyclopropanecarboxamido)pyridin-4-yl)amino)-3-methoxypyridin-2-yl)-1-(bicyclo[1.1.1]pentan-1-yl)-2-oxo-1,2,3,5-tetrahydro-4H-benzo[e][1,4]diazepin-4-yl)methyl)-N,N-dimethylpicolinamide

A 20 mL scintillation vial was charged with N-(5-acetyl-4-((2-chloro-3-methoxypyridin-4-yl)amino)pyridin-2-yl)cyclopropanecarboxamide (Int. 16) (34 mg, 94 μmol), 6-((1-(bicyclo-[1.1.1]pentan-1-yl)-2-oxo-8-(4,4,5,5-tetramethyl-1,3,2-dioxaborolan-2-yl)-1,2,3,5-tetrahydro-4H-benzo[e][1,4]diazepin-4-yl)methyl)-N,N-dimethylpicolinamide (49 mg, 94 μmol), potassium phosphate tribasic (60 mg, 0.28 mmol) and [1,1′-bis(di-tert-butylphosphino)ferrocene]dichloro-palladium(II) 6.1 mg, 9.4 μmol). The mixture was dissolved in 1,4-dioxane (2 mL) and water (250 μL), was sparged with nitrogen gas for 5 minutes and sealed. Then the reaction mixture was heated to 90° C. for 16 hours. The product mixture was diluted with water (30 mL) and extracted with EtOAc (3×30 mL). The combined organic layers were dried over Na₂SO₄, filtered and concentrated under reduced pressure. The residue obtained was purified by prep-HPLC on a C₁₈ column (15-40% MeCN in 0.05% TFA(aq)) to afford the TFA salt of the title compound (29 mg, 43% yield). LC-MS calc. for C₄₀H₄₃N₈O₅ [M+H]⁺: m/z=715.3; Found: 715.5.

### Examples 177-178

Examples 177-178 are shown below in Table 18 and were prepared in accordance with the synthetic protocols set forth in Example 176 Steps 1 through 8 using the appropriate intermediates, as well as commercial starting materials. The following compounds are TFA salts unless otherwise noted.

TABLE 18
Examples 177-178

| | | | Analytical Data (LCMS |
|---|---|---|---|
| Ex. | Name | Structure | and/or NMR) |
| 177 | 6-((8-(4-((5-acetyl-2- (cyclopropane- carboxamido) pyridin-4- yl)amino)-3- methoxypyridin-2-yl)- 1-(3-fluorobicyclo- [1.1.1]pentan-1-yl)-2- oxo-1,2,3,5- tetrahydro-4H- pyrido[3,2-e][1,4] diazepin-4- yl)methyl)-N,N- dimethylpicolinamide | 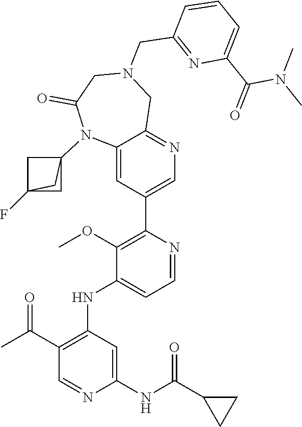 [SMILES: COc1c(Nc2cc(NC(=O)C3CC3)ncc2C(C)=O)ccnc1-c1cnc2c(c1)N(C13CC(F)(C1)C3)C(=O)CN(Cc1cccc(C(=O)N(C)C)n1)C2] | LCMS calc. for C₃₉H₄₁FN₉O₅ [M + H]⁺: m/z = 734.3; Found: 734.4. |
| 178 | 6-((8-(4-((5-acetyl-2- ((1S,2R)-2- fluorocyclopropane-1- carboxamido)pyridin- 4-yl)amino)-3- methoxypyridin-2-yl)- 1-(bicyclo[1.1.1]- pentan-1-yl)-2-oxo- 1,2,3,5-tetrahydro-4H- pyrido[3,2-e][1,4] diazepin-4- yl)methyl)-N,N- dimethylpicolinamide | 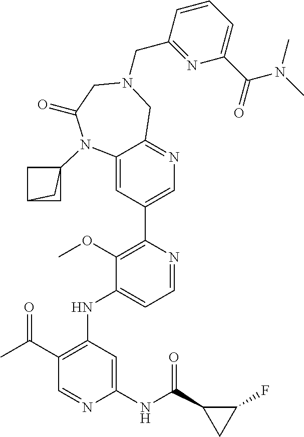 [SMILES: COc1c(Nc2cc(NC(=O)[C@@H]3C[C@H]3F)ncc2C(C)=O)ccnc1-c1cnc2c(c1)N(C13CC(C1)C3)C(=O)CN(Cc1cccc(C(=O)N(C)C)n1)C2] | LCMS calc. for C₃₉H₄₁FN₉O₅ [M + H]⁺: m/z = 734.3; Found: 734.4. |

### Example 179. 6-((8-(3-((5-Acetyl-2-(cyclopropanecarboxamido)pyridin-4-yl)amino)-2-methoxyphenyl)-1-(2-oxabicyclo[2.1.1]hexan-4-yl)-2-oxo-1,2,3,5-tetrahydro-4H-pyrido[3,2-e][1,4]diazepin-4-yl)methyl)-N,N-dimethylpicolinamide

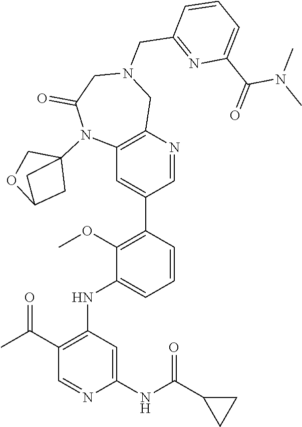 [SMILES: COc1c(Nc2cc(NC(=O)C3CC3)ncc2C(C)=O)cccc1-c1cnc2c(c1)N(C13COC(C1)C3)C(=O)CN(Cc1cccc(C(=O)N(C)C)n1)C2]

### Step 1. 6-((1-(2-Oxabicyclo[2.1.1]hexan-4-yl)-8-bromo-2-oxo-1,2,3,5-tetrahydro-4H-pyrido[3,2-e][1,4]diazepin-4-yl)methyl)-N,N-dimethylpicolinamide

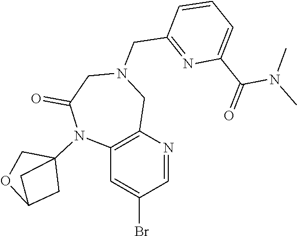 [SMILES: CN(C)C(=O)c1cccc(CN2CC(=O)N(C34COC(C3)C4)c3cc(Br)cnc3C2)n1]

The title compound was prepared in accordance with the synthetic protocols set forth in Example 176 Steps 1 through 6 using appropriate intermediates, as well as commercial starting materials. LCMS calc. for C₂₂H₂₅BrN₅O₃[M+H]⁺: m/z=486.1; Found: 486.2.

### Step 2. 6-((8-(3-((5-Acetyl-2-(cyclopropanecarboxamido)pyridin-4-yl)amino)-2-methoxyphenyl)-1-(2-oxabicyclo[2.1.1]hexan-4-yl)-2-oxo-1,2,3,5-tetrahydro-4H-pyrido[3,2-e][1,4]diazepin-4-yl)methyl)-N,N-dimethylpicolinamide

A mixture of [3-[[5-acetyl-2-(cyclopropanecarbonylamino)-4-pyridinyl]amino]-2-methoxyphenyl]boronic acid (7.89 mg, 0.02 mmol), 6-((1-(2-oxabicyclo[2.1.1]hexan-4-yl)-8-bromo-2-oxo-1,2,3,5-tetrahydro-4H-pyrido[3,2-e][1,4]diazepin-4-yl)methyl)-N,N-dimethylpicolinamide (8.0 mg, 0.02 mmol), potassium carbonate (11.4 mg, 0.08 mmol) and [1,1′-bis(diphenylphosphino)ferrocene]dichloropalladium(II) complex with dichloromethane (2.02 mg, 2.7 μmol) in 1,4-dioxane (1 mL) and water (100 μL) was degassed and stirred at 90° C. for 2 h. The mixture was purified on prepLCMS to give the title compound (5.8 mg, 7.94 μmol, 48% yield). LCMS calc. for C₄₀H₄₃N₈O₆ [M+H]⁺: m/z=731.3; Found: 731.4.

### Example 180. 6-((8-(4-((5-acetyl-2-(cyclopropanecarboxamido)pyridin-4-yl)amino)-3-methoxypyridin-2-yl)-1-isobutyl-2-oxo-1,2,3,5-tetrahydro-4H-pyrido[3,2-e][1,4]diazepin-4-yl)methyl)-N,N-dimethylpicolinamide

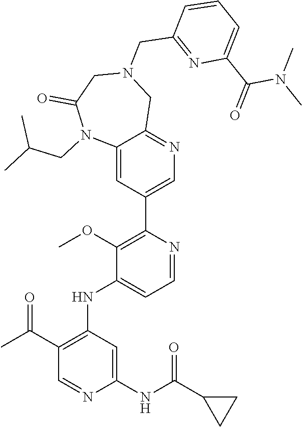 [SMILES: COc1c(Nc2cc(NC(=O)C3CC3)ncc2C(C)=O)ccnc1-c1cnc2c(c1)N(CC(C)C)C(=O)CN(Cc1cccc(C(=O)N(C)C)n1)C2]

### Step 1. 8-bromo-1-isobutyl-1,3,4,5-tetrahydro-2H-pyrido[3,2-e][1,4]diazepin-2-one

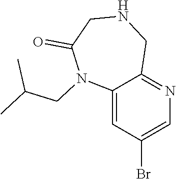 [SMILES: CC(C)CN1C(=O)CNCc2ncc(Br)cc21]

The title compound was prepared in accordance with the synthetic protocols set forth in Example 165 Steps 1 through 3 using the appropriate intermediates and commercial starting materials. LC-MS calc. for C₁₂H₁₇BrN₃O [M+H]⁺: m/z=298.1/300.1; Found: 298.0/300.0.

### Step 2. 6-((8-(4-((5-acetyl-2-(cyclopropanecarboxamido)pyridin-4-yl)amino)-3-methoxypyridin-2-yl)-1-isobutyl-2-oxo-1,2,3,5-tetrahydro-4H-pyrido[3,2-e][1,4]diazepin-4-yl)methyl)-N,N-dimethylpicolinamide

The title compound was prepared in accordance with the synthetic protocols set forth in Example 176 Steps 6-8 using the appropriate intermediates and commercial starting materials. LC-MS calc. for C₃₈H₄₄N₉O₅ [M+H]⁺: m/z=706.3; Found: 706.6. ¹H NMR (300 MHz, CDCl₃) δ 11.72 (s, 1H), 9.12 (s, 1H), 8.82 (s, 1H), 8.55-8.50 (m, 2H), 8.42 (s, 1H), 8.25 (s, 1H), 7.90-7.80 (m, 1H), 7.70-7.60 (m, 2H), 7.58 (d, J=7.5 Hz, 1H), 4.11 (s, 2H), 4.06 (s, 2H), 3.89 (d, J=7.1 Hz, 2H), 3.67 (s, 3H), 3.33 (s, 2H), 3.17 (s, 3H), 3.15 (s, 3H), 2.73 (s, 3H), 2.05-1.95 (m, 1H), 1.65-1.60 (m, 1H), 1.20-1.15 (m, 2H), 1.05-1.00 (m, 2H), 0.98 (t, J=8.9 Hz, 6H).

### Example 181. 6-((8-(4-((5-Acetyl-2-(cyclopropanecarboxamido)pyridin-4-yl)amino)-3-methoxypyridin-2-yl)-1-cyclopentyl-2-oxo-1,2,3,5-tetrahydro-4H-pyrido[3,2-e][1,4]diazepin-4-yl)methyl)-N,N-dimethylpicolinamide

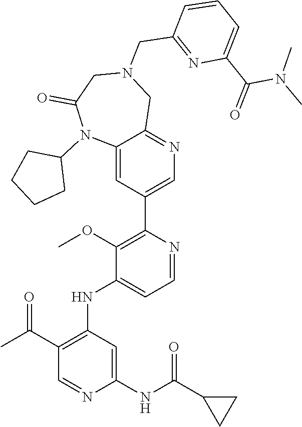 [SMILES: COc1c(Nc2cc(NC(=O)C3CC3)ncc2C(C)=O)ccnc1-c1cnc2c(c1)N(C1CCCC1)C(=O)CN(Cc1cccc(C(=O)N(C)C)n1)C2]

The title compound was prepared as a TFA salt in accordance with the synthetic protocols set forth in Example 180 using the appropriate intermediates and commercial starting materials. LC-MS calc. for C₃₉H₄₄N₉O₅ [M+H]⁺: m/z=718.3; Found: 718.6.

### Example 182. 6-((8-(3-((5-Acetyl-2-((1R,2S)-2-fluorocyclopropane-1-carboxamido)pyridin-4-yl)amino)-2-methoxyphenyl)-2-oxo-1-((S)-tetrahydro-2H-pyran-3-yl)-1,2,3,5-tetrahydro-4H-pyrido[3,2-e][1,4]diazepin-4-yl)methyl)-N,N-dimethylpicolinamide

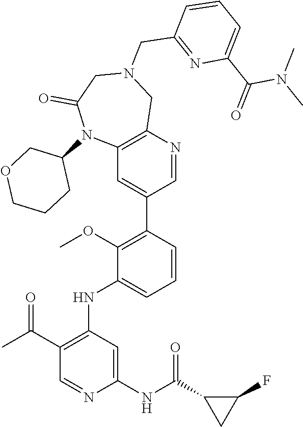 [SMILES: COc1c(Nc2cc(NC(=O)[C@H]3C[C@@H]3F)ncc2C(C)=O)cccc1-c1cnc2c(c1)N([C@H]1CCCOC1)C(=O)CN(Cc1cccc(C(=O)N(C)C)n1)C2]

### Step 1. (R)-Tetrahydro-2H-pyran-3-yl 4-nitrobenzenesulfonate

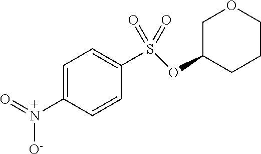

To a mixture of (3R)-oxan-3-ol (50.0 mg, 0.49 mmol) in DCM (1 mL) was added triethylamine (0.14 mL, 0.98 mmol) followed by a solution of 4-nitrobenzenesulfonyl chloride (162.74 mg, 0.73 mmol) in DCM cooled in an ice bath. The mixture was stirred at rt overnight. The reaction was washed with water, concentrated and purified on FCC (0-60% EtOAc/Hex) to give the title compound (140 mg, 99.5% yield). The product does not ionize.

### Step 2. tert-Butyl (S)-8-bromo-2-oxo-1-(tetrahydro-2H-pyran-3-yl)-1,2,3,5-tetrahydro-4H-pyrido[3,2-e][1,4]diazepine-4-carboxylate

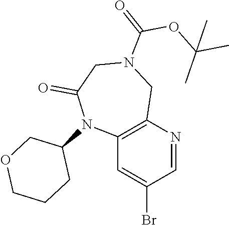 [SMILES: CC(C)(C)OC(=O)N1CC(=O)N([C@H]2CCCOC2)c2cc(Br)cnc2C1]

A mixture of tert-butyl 8-bromo-2-oxo-1,2,3,5-tetrahydro-4H-pyrido[3,2-e][1,4]-diazepine-4-carboxylate (Int. 32) (166.8 mg, 0.49 mmol), cesium carbonate (476.3 mg, 1.46 mmol) and (R)-tetrahydro-2H-pyran-3-yl 4-nitrobenzenesulfonate (140.0 mg, 0.49 mmol) in 1,4-dioxane (2 mL) was stirred at 70° C. overnight. The mixture was diluted with DCM, washed with water, concentrated and purified on FCC (0-100% EtOAc/Hexanes) to give the title compound (40 mg, 19.3% yield). LCMS calc. for C₁₈H₂₅BrN₃O₄[M+H]⁺: m/z=426.1; Found: 426.2.

### Step 3. 6-((8-(3-((5-Acetyl-2-((1R,2S)-2-fluorocyclopropane-1-carboxamido)pyridin-4-yl)amino)-2-methoxyphenyl)-2-oxo-1-((S)-tetrahydro-2H-pyran-3-yl)-1,2,3,5-tetrahydro-4H-pyrido[3,2-e][1,4]diazepin-4-yl)methyl)-N,N-dimethylpicolinamide

The title compound was prepared in accordance with the synthetic protocols set forth in Example 176 Step 6 and Example 179 Step 2 using appropriate intermediates, as well as commercial starting materials. LCMS calc. for C₄₀H₄₄FN₈O₆[M+H]⁺: m/z=751.3; Found: 751.4.

### Example 183. (S)-6-((8-(4-((5-acetyl-2-(cyclopropanecarboxamido)pyridin-4-yl)amino)-3-methoxypyridin-2-yl)-1-(sec-butyl)-2-oxo-1,2,3,5-tetrahydro-4H-pyrido[3,2-e][1,4]diazepin-4-yl)methyl)-N,N-dimethylpicolinamide

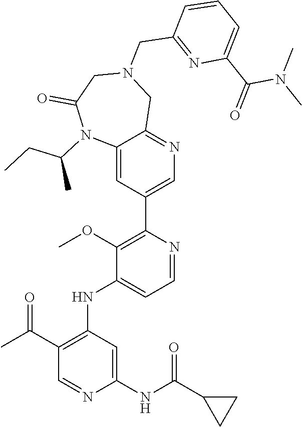 [SMILES: CC[C@H](C)N1C(=O)CN(Cc2cccc(C(=O)N(C)C)n2)Cc2ncc(-c3nccc(Nc4cc(NC(=O)C5CC5)ncc4C(C)=O)c3OC)cc21]

### Step 1. (S)-6-((8-bromo-1-(sec-butyl)-2-oxo-1,2,3,5-tetrahydro-4H-pyrido[3,2-e][1,4]diazepin-4-yl)methyl)-N,N-dimethylpicolinamide

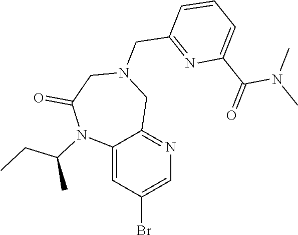 [SMILES: CC[C@H](C)N1C(=O)CN(Cc2cccc(C(=O)N(C)C)n2)Cc2ncc(Br)cc21]

The title compound was prepared in accordance with the synthetic protocols set forth in Example 182 Steps 1-2 using the appropriate intermediates and commercial starting materials. LC-MS calc. for C₂₁H₂₇BrN₅O₂[M+H]⁺: m/z=460.1; Found: 460.3.

### Step 2. (S)-6-((8-(4-((5-acetyl-2-(cyclopropanecarboxamido)pyridin-4-yl)amino)-3-methoxypyridin-2-yl)-1-(sec-butyl)-2-oxo-1,2,3,5-tetrahydro-4H-pyrido[3,2-e][1,4]diazepin-4-yl)methyl)-N,N-dimethylpicolinamide

The title compound was prepared as a TFA salt in accordance with the synthetic protocols set forth in Example 176 Steps 7-8 using the appropriate intermediates and commercial starting materials. LC-MS calc. for C₃₈H₄₄N₉O₅ [M+H]⁺: m/z=706.3; Found: 706.6.

### Examples 184-185

Examples 184-185 are shown below in Table 19 and were prepared in accordance with the synthetic protocols set forth in Example 183 using the appropriate intermediates, as well as commercial starting materials. The following compounds are TFA salts unless otherwise noted.

TABLE 19
Examples 184-185

| | | | Analytical Data |
|---|---|---|---|
| | | | (LCMS and/or |
| Ex. | Name | Structure | NMR) |
| 184 | (R)-6-((8-(4-((5-acetyl-2- (cyclopropanecarboxamido) pyridin-4-yl)amino)-3- methoxypyridin-2-yl)-1-(sec- butyl)-2-oxo-1,2,3,5- tetrahydro-4H-pyrido[3,2- e][1,4]diazepin-4-yl)methyl)- N,N-dimethylpicolinamide | 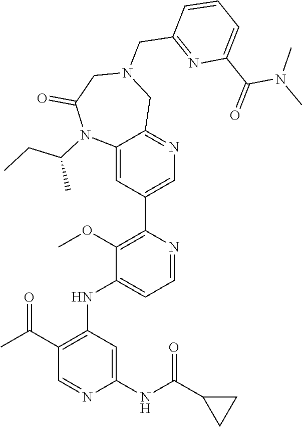 [SMILES: CC[C@@H](C)N1C(=O)CN(Cc2cccc(C(=O)N(C)C)n2)Cc2ncc(-c3nccc(Nc4cc(NC(=O)C5CC5)ncc4C(C)=O)c3OC)cc21] | LC-MS calc. for C₃₈H₄₄N₉O₅ [M + H]⁺: m/z = 706.3; Found: 706.5. |
| 185 | 6-((8-(4-((5-acetyl-2- ((1S,2R)-2- fluorocyclopropane-1- carboxamido)pyridin-4- yl)amino)-3-methoxypyridin- 2-yl)-1-((R)-sec-butyl)-2- oxo-1,2,3,5-tetrahydro-4H- pyrido[3,2-e][1,4]diazepin-4- yl)methyl)-N,N- dimethylpicolinamide | 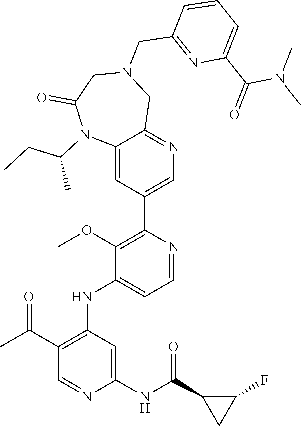 [SMILES: CC[C@@H](C)N1C(=O)CN(Cc2cccc(C(=O)N(C)C)n2)Cc2ncc(-c3nccc(Nc4cc(NC(=O)[C@@H]5C[C@H]5F)ncc4C(C)=O)c3OC)cc21] | LC-MS calc. for C₃₈H₄₃FN₉O₅ [M + H]+: m/z = 724.3; Found: 724.5. |

### Example 186. 5-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylbicyclo[3.1.1]heptane-1-carboxamide

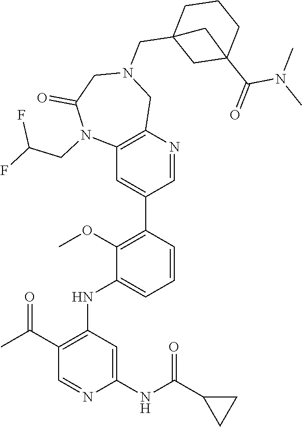 [SMILES: COc1c(Nc2cc(NC(=O)C3CC3)ncc2C(C)=O)cccc1-c1cnc2c(c1)N(CC(F)F)C(=O)CN(CC13CCCC(C(=O)N(C)C)(C1)C3)C2]

### Step 1. Methyl 5-(dimethylcarbamoyl)bicyclo[3.1.1]heptane-1-carboxylate

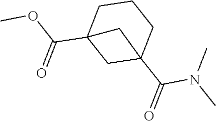

To a mixture of 5-methoxycarbonylbicyclo[3.1.1]heptane-1-carboxylic acid (114 mg, 0.58 mmol), dimethylamine HCl (61 mg, 0.75 mmol) and 1-[bis(dimethylamino)methylene]-1H-1,2,3-triazolo[4,5-b]pyridinium 3-oxid hexafluorophosphate (241 mg, 0.63 mmol) in DCM (2 mL) was added N,N-diisopropylethylamine (301 μL, 1.73 mmol). The mixture was stirred at rt for 1 h. The mixture was washed with water, concentrated and purified on prepLCMS on a C₁₈ column (16.2-36.2% MeCN in 0.2% TFA(aq)) to give the title compound (120 mg, 0.53 mmol, 93% yield). LCMS calc. for C₁₂H₂₀NO₃ [M+H]⁺: m/z=226.1; Found: 226.2.

### Step 2. 5-(Hydroxymethyl)-N,N-dimethylbicyclo[3.1.1]heptane-1-carboxamide

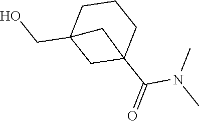

To a mixture of methyl 5-(dimethylcarbamoyl)bicyclo[3.1.1]heptane-1-carboxylate (60.0 mg, 0.27 mmol) in THF (2 mL) and ethanol (1 mL) was added lithium borohydride (0.4 mL, 0.8 mmol, 2.0 M in THF) dropwise. The mixture was stirred at rt for 1 h. The mixture was diluted with water and purified on prepLCMS on a C₁₈ column (8.4-28.4% MeCN in 0.2% TFA(aq)) to give the title compound (50 mg, 0.25 mmol, 95.2% yield) LCMS calc. for C₁₁H₂₀NO₂ [M+H]⁺: m/z=198.1; Found: 198.2.

### Step 3. 5-Formyl-N,N-dimethylbicyclo[3.1.1]heptane-1-carboxamide

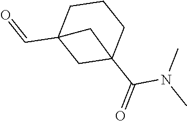

To a mixture of 5-(hydroxymethyl)-N,N-dimethylbicyclo[3.1.1]heptane-1-carboxamide (50.0 mg, 0.25 mmol) in DCM (2 mL) was added Dess-Martin periodinane (215 mg, 0.51 mmol). The mixture was stirred at rt for 1 h. The mixture was diluted with water and purified on prepLCMS to give the title compound (6 mg, 0.031 mmol, 12% yield). LCMS calc. for C₁₁H₁₈NO₂ [M+H]⁺: m/z=196.1; Found: 196.2.

### Step 4. 5-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylbicyclo[3.1.1]heptane-1-carboxamide

The title compound was prepared in accordance with the synthetic protocols set forth in Example 176 Step 6 and Example 179 Step 2 using appropriate intermediates, as well as commercial starting materials. LCMS calc. for C₃₉H₄₆F₂N₇O₅[M+H]⁺: m/z=730.4; Found: 730.4.

### Example 187. 6-((8-(4-((5-Acetyl-2-(cyclopropanecarboxamido)pyridin-4-yl)amino)-3-methoxypyridin-2-yl)-1-cyclopentyl-2-oxo-1,2,3,5-tetrahydro-4H-benzo[e][1,4]diazepin-4-yl)methyl)-N,N-dimethylpicolinamide

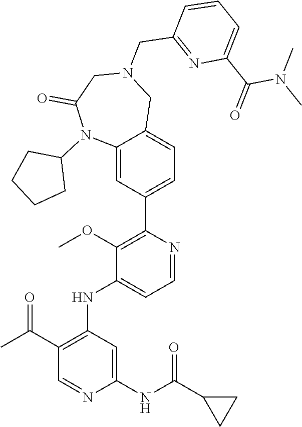 [SMILES: COc1c(Nc2cc(NC(=O)C3CC3)ncc2C(C)=O)ccnc1-c1ccc2c(c1)N(C1CCCC1)C(=O)CN(Cc1cccc(C(=O)N(C)C)n1)C2]

### Step 1. 6-((8-bromo-1-cyclopentyl-2-oxo-1,2,3,5-tetrahydro-4H-benzo[e][1,4]diazepin-4-yl)methyl)-N,N-dimethylpicolinamide

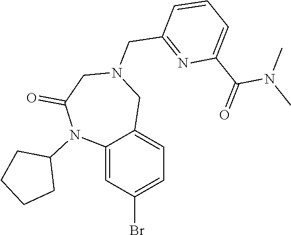 [SMILES: CN(C)C(=O)c1cccc(CN2CC(=O)N(C3CCCC3)c3cc(Br)ccc3C2)n1]

The title compound was prepared in accordance with the synthetic protocols set forth in Example 103 Step 1 using appropriate intermediates, as well as commercial starting materials. LCMS calc. for C₂₃H₂₈BrN₄O₂[M+H]⁺: m/z=470.1/472.1; Found: 470.5/472.2.

### Step 2. 6-((8-(4-((5-acetyl-2-(cyclopropanecarboxamido)pyridin-4-yl)amino)-3-methoxypyridin-2-yl)-1-cyclopentyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 176 Steps 7 through 8 using appropriate intermediates, as well as commercial starting materials. LCMS calc. for C₄₀H₄₅N₈O₅ [M+H]⁺: m/z=717.4; Found: 717.4.

### Example 188: 6-((8-(4-((5-acetyl-2-(cyclopropanecarboxamido)pyridin-4-yl)amino)-3-methoxypyridin-2-yl)-2-oxo-1-propyl-1,2,3,5-tetrahydro-4H-pyrido[3,2-e][1,4]diazepin-4-yl)methyl)-N,N-dimethylpicolinamide

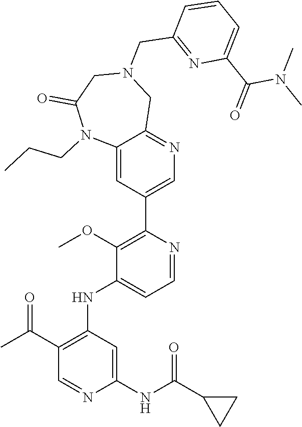 [SMILES: CCCN1C(=O)CN(Cc2cccc(C(=O)N(C)C)n2)Cc2ncc(-c3nccc(Nc4cc(NC(=O)C5CC5)ncc4C(C)=O)c3OC)cc21]

### Step 1. 6-((8-Bromo-2-oxo-1-propyl-1,2,3,5-tetrahydro-4H-pyrido[3,2-e][1,4]diazepin-4-yl)methyl)-N,N-dimethylpicolinamide

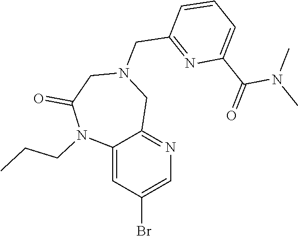 [SMILES: CCCN1C(=O)CN(Cc2cccc(C(=O)N(C)C)n2)Cc2ncc(Br)cc21]

The title compound was prepared in accordance with the synthetic protocols set forth in Example 36 Step 1 using the appropriate intermediates and commercial starting materials. LC-MS calc. for C₂₀H₂₅BrN₅O₂[M+H]⁺: m/z=446.1; Found: 446.2.

### Step 2. 6-((8-(4-((5-acetyl-2-(cyclopropanecarboxamido)pyridin-4-yl)amino)-3-methoxypyridin-2-yl)-2-oxo-1-propyl-1,2,3,5-tetrahydro-4H-pyrido[3,2-e][1,4]diazepin-4-yl)methyl)-N,N-dimethylpicolinamide

The title compound was prepared as a TFA salt in accordance with the synthetic protocols set forth in Example 176 Steps 7-8 using the appropriate intermediates and commercial starting materials. LC-MS calc. for C₃₇H₄₂N₉O₅ [M+H]⁺: m/z=692.3; Found: 692.6.

### Example 189. 6-((8-(4-((5-acetyl-2-(cyclopropanecarboxamido)pyridin-4-yl)amino)-3-methoxypyridin-2-yl)-2-oxo-1-(pentan-3-yl)-1,2,3,5-tetrahydro-4H-pyrido[3,2-e][1,4]diazepin-4-yl)methyl)-N,N-dimethylpicolinamide

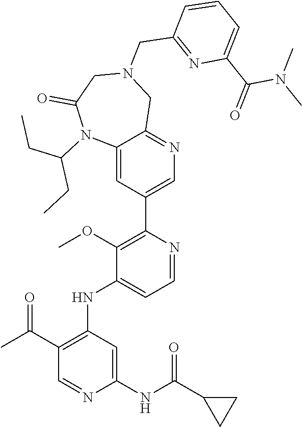 [SMILES: CCC(CC)N1C(=O)CN(Cc2cccc(C(=O)N(C)C)n2)Cc2ncc(-c3nccc(Nc4cc(NC(=O)C5CC5)ncc4C(C)=O)c3OC)cc21]

The title compound was prepared in accordance with the synthetic protocols set forth in Example 188 using the appropriate intermediates and commercial starting materials. LC-MS calc. for C₃₉H₄₆N₉O₅ [M+H]⁺: m/z=720.4; Found: 720.4. ¹H NMR (300 MHz, CDCl₃) δ 12.89 (s, 1H), 12.21 (s, 1H), 9.21 (s, 1H), 8.85-8.80 (m, 1H), 8.80-8.75 (m, 2H), 8.30 (s, 1H), 8.00-7.90 (m, 1H), 7.80-7.75 (m, 1H), 7.70-7.60 (m, 2H), 4.61 (s, 4H), 4.40-4.35 (m, 1H), 3.93 (s, 2H), 3.74 (s, 3H), 3.18 (s, 3H), 3.09 (s, 3H), 2.77 (s, 3H), 2.10-2.00 (s, 1H), 2.00-1.80 (m, 4H), 1.20-1.10 (m, 2H), 1.10-1.05 (m, 2H), 1.00-0.90 (m, 6H).

### Example 190. 6-((8-(3-((5-Acetyl-2-(cyclopropanecarboxamido)pyridin-4-yl)amino)-2-methoxyphenyl)-1-cyclohexyl-2-oxo-1,2,3,5-tetrahydro-4H-benzo[e][1,4]diazepin-4-yl)methyl)-N,N-dimethylpicolinamide

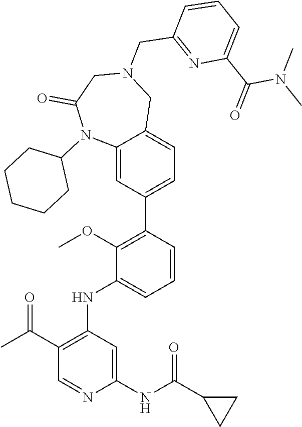 [SMILES: COc1c(Nc2cc(NC(=O)C3CC3)ncc2C(C)=O)cccc1-c1ccc2c(c1)N(C1CCCCC1)C(=O)CN(Cc1cccc(C(=O)N(C)C)n1)C2]

### Step 1. 4-Bromo-2-(cyclohexylamino)benzonitrile

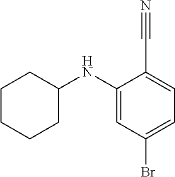 [SMILES: N#Cc1ccc(Br)cc1NC1CCCCC1]

Cyclohexylamine (3.43 mL, 30 mmol) was added to a stirring solution of 4-bromo-2-fluorobenzonitrile (2.0 g, 10 mmol) and N,N-diisopropylethylamine (5.23 mL, 30 mmol) in N-methyl-2-pyrrolidone (40 mL). The reaction mixture was heated to 110° C. and stirred for 16 hours. The product mixture was diluted with water (150 mL), extracted with EtOAc (150 mL), and washed with sat. brine (aq) solution (3×100 mL). The organic layer was dried over Na₂SO₄, filtered and concentrated under reduced pressure. The residue obtained was purified by silica gel column chromatography (0-5% EtOAc/DCM) to yield the title compound (2.5 g, 89% yield). LCMS calc. for C₁₃H₁₆BrN₂ [M+H]⁺: m/z=279.1/281.1; Found: 278.9/280.9.

### Step 2. 2-(Aminomethyl)-5-bromo-N-cyclohexylaniline

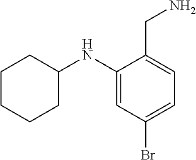 [SMILES: NCc1ccc(Br)cc1NC1CCCCC1]

Borane dimethyl sulfide complex (4.48 mL, 44.8 mmol) was added dropwise to a stirring solution of 4-bromo-2-(cyclohexylamino)benzonitrile (2.5 g, 8.95 mmol) in THF (25 mL) at 10° C. The reaction mixture was stirred at 10° C. for 16 hours. The product mixture was diluted with water (100 mL) and extracted with EtOAc (3×100 mL). The combined organic layers were dried over Na₂SO₄, filtered and concentrated under reduced pressure. The residue obtained was purified by silica gel column chromatography (0-5% MeOH/DCM) to yield the title compound (1.1 g, 43% yield). LCMS calc. for C₁₃H₁₇BrN [M−H₂N]⁺: m/z=266.1/268.1; Found: 265.9/267.8.

### Step 3. tert-Butyl (4-bromo-2-(cyclohexylamino)benzyl)glycinate

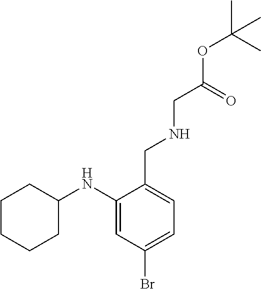 [SMILES: CC(C)(C)OC(=O)CNCc1ccc(Br)cc1NC1CCCCC1]

tert-Butyl bromoacetate was added to a stirring solution of 2-(aminomethyl)-5-bromo-N-cyclohexylaniline (1.4 g, 4.9 mmol) and N,N-diisopropylethylamine (1.03 mL, 5.93 mmol) in dimethylacetamide (20 mL). The reaction mixture was heated to 30° C. for 2 hours. The product mixture was concentrated under reduced pressure. The residue obtained was purified by silica gel column chromatography (0-5% EtOAc/DCM) to yield the title compound (1.1 g, 56% yield). LCMS calc. for C₁₉H₃₀BrN₂O₂[M+H]⁺: m/z=397.2/399.2; Found: 397.0/399.3.

### Step 4. tert-Butyl N-(4-bromo-2-(cyclohexylamino)benzyl)-N-(tert-butoxycarbonyl)-glycinate

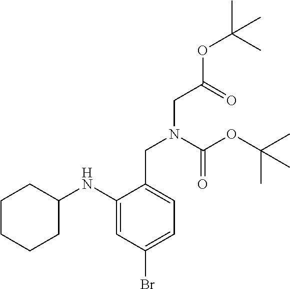 [SMILES: CC(C)(C)OC(=O)CN(Cc1ccc(Br)cc1NC1CCCCC1)C(=O)OC(C)(C)C]

Di-tert-butyl dicarbonate (827 μL, 3.6 mmol) was added to a stirring solution of tert-butyl (4-bromo-2-(cyclohexylamino)benzyl)glycinate (1.3 g, 3.3 mmol) and N,N-diisopropylethylamine (683 μL, 3.93 mmol) in DCM (30 mL) at 10° C. The reaction mixture was stirred for 16 hours. The product mixture was concentrated under reduced pressure. The residue obtained was purified by silica gel column chromatography (0-5% EtOAc/DCM) to yield the title compound (1.4 g, 86% yield). LCMS calc. for C₂₄H₃₈BrN₂O₄[M+H]⁺: m/z=497.2/499.2; Found: 497.3/499.3.

### Step 5. N-(4-Bromo-2-(cyclohexylamino)benzyl)-N-(tert-butoxycarbonyl)glycine

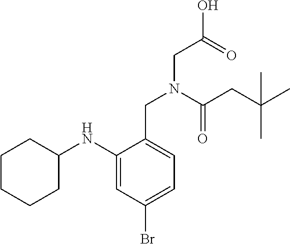 [SMILES: CC(C)(C)CC(=O)N(CC(=O)O)Cc1ccc(Br)cc1NC1CCCCC1]

Sodium hydroxide (338 mg, 14.1 mmol) was added to tert-butyl N-(4-bromo-2-(cyclohexylamino)benzyl)-N-(tert-butoxycarbonyl)glycinate (700 mg, 1.41 mmol) in MeOH (20 mL) at 15° C. The reaction mixture was stirred at 15° C. for 16 hours. The product mixture was concentrated under reduced pressure to yield the title compound (500 mg, 1.13 mmol) which was used without further purification. LCMS calc. for C₂₀H₃₀BrN₂O₄[M+H]⁺: m/z=441.1/443.1; Found: 440.9/442.8.

### Step 6. tert-Butyl 8-bromo-1-cyclohexyl-2-oxo-1,2,3,5-tetrahydro-4H-benzo[e][1,4]-diazepine-4-carboxylate

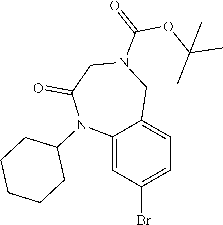 [SMILES: CC(C)(C)OC(=O)N1CC(=O)N(C2CCCCC2)c2cc(Br)ccc2C1]

Triethylamine (789 μL, 5.66 mmol) was added to a stirring solution N-(4-bromo-2-(cyclohexylamino)benzyl)-N-(tert-butoxycarbonyl)glycine (500 mg, 1.13 mmol) and 2-chloro-1-methylpyridinium iodide (2.91 g, 11.3 mmol) in 1,2-dichloroethane (20 mL). The reaction mixture was heated to 60° C. for 2 hours. The product mixture was diluted with water (50 mL) and extracted with EtOAc (3×50 mL). The combined organic layers were dried over Na₂SO₄, filtered and concentrated under reduced pressure. The residue obtained was purified by silica gel column chromatography (0-5% EtOAc/DCM) to yield the title compound (200 mg, 42% yield). LCMS calc. for C₂₀H₂₇BrN₂NaO₃ [M+Na]⁺: m/z=445.1/447.1; Found: 444.8/446.7.

### Step 7. 6-((8-Bromo-1-cyclohexyl-2-oxo-1,2,3,5-tetrahydro-4H-benzo[e][1,4]diazepin-4-yl)methyl)-N,N-dimethylpicolinamide

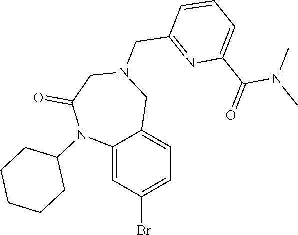 [SMILES: CN(C)C(=O)c1cccc(CN2CC(=O)N(C3CCCCC3)c3cc(Br)ccc3C2)n1]

The title compound was prepared in accordance with the synthetic protocols set forth in Example 176 Step 6 using appropriate intermediates, as well as commercial starting materials. LCMS calc. for C₂₄H₃₀BrN₄O₂[M+H]⁺: m/z=485.2/487.2; Found: 484.7/486.6.

### Step 8. 6-((8-(3-((5-Acetyl-2-(cyclopropanecarboxamido)pyridin-4-yl)amino)-2-methoxyphenyl)-1-cyclohexyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 84 Step 2 using appropriate intermediates, as well as commercial starting materials. LCMS calc. for C₄₂H₄₈N₇O₅ [M+H]⁺: m/z=730.4; Found: 730.2. ¹H NMR (600 MHz, DMSO-d₆) δ 11.11 (s, 1H), 11.05 (s, 1H), 8.85 (s, 1H), 8.05 (t, J=7.8 Hz, 1H), 7.92 (s, 1H), 7.66 (d, J=8.6 Hz, 2H), 7.59 (dd, J=7.8, 2.5 Hz, 3H), 7.52 (dd, J=7.2, 2.3 Hz, 1H), 7.38-7.32 (m, 2H), 4.57-4.21 (m, 5H), 3.53 (s, 2H), 3.36 (s, 3H), 3.03 (s, 3H), 2.94 (s, 3H), 2.66 (s, 3H), 2.04 (s, 1H), 2.02-1.99 (m, 1H), 1.85 (d, J=12.6 Hz, 1H), 1.82-1.73 (m, 2H), 1.64-1.54 (m, 3H), 1.33 (s, 2H), 1.06 (d, J=12.7 Hz, 1H), 0.81 (ddd, J=21.2, 8.3, 3.9 Hz, 4H).

### Example 191. 6-((8-(4-((5-Acetyl-2-(cyclopropanecarboxamido)pyridin-4-yl)amino)-3-methoxypyridin-2-yl)-1-cyclohexyl-2-oxo-1,2,3,5-tetrahydro-4H-pyrido[3,2-e][1,4]diazepin-4-yl)methyl)-N,N-dimethylpicolinamide

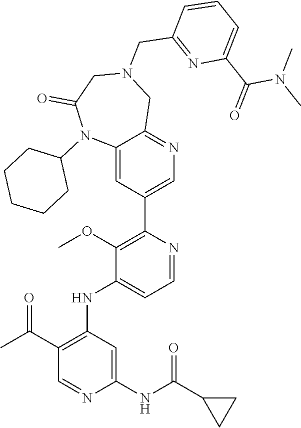 [SMILES: COc1c(Nc2cc(NC(=O)C3CC3)ncc2C(C)=O)ccnc1-c1cnc2c(c1)N(C1CCCCC1)C(=O)CN(Cc1cccc(C(=O)N(C)C)n1)C2]

### Step 1. tert-Butyl 8-bromo-1-(cyclohex-2-en-1-yl)-2-oxo-1,2,3,5-tetrahydro-4H-pyrido[3,2-e][1,4]diazepine-4-carboxylate

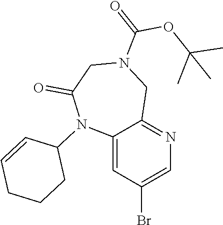 [SMILES: CC(C)(C)OC(=O)N1CC(=O)N(C2C=CCCC2)c2cc(Br)cnc2C1]

To a stirring solution of tert-butyl 8-bromo-2-oxo-1,2,3,5-tetrahydro-4H-pyrido[3,2-e][1,4]diazepine-4-carboxylate (Int. 32) (200 mg, 0.58 mmol) in DMF (1 mL) was added cesium carbonate (571 mg, 1.75 mmol) and 3-bromocyclohexene (2350 mg, 14.6 mmol) sequentially. The reaction mixture was heated to 60° C. and stirred for 2 hours. The product mixture was diluted with brine (30 mL) and extracted with a 3:1 CHCl₃/iPrOH mixture (3×30 mL). The combined organic layers were dried over MgSO₄, filtered and concentrated under reduced pressure. The residue obtained was purified by silica gel column chromatography (0-5% MeOH/DCM) to yield the title compound (200 mg, 81% yield). LC-MS calc. for C₁₉H₂₄BrN₃NaO₃ [M+Na]⁺: m/z=444.1/446.1; Found: 444.1/446.1.

### Step 2. tert-Butyl 8-bromo-1-cyclohexyl-2-oxo-1,2,3,5-tetrahydro-4H-pyrido[3,2-e][1,4]diazepine-4-carboxylate

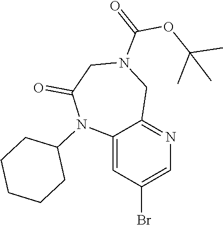 [SMILES: CC(C)(C)OC(=O)N1CC(=O)N(C2CCCCC2)c2cc(Br)cnc2C1]

Platinum on carbon (60 mg, 31 μmol, 10 wt %) was added to a solution of tert-butyl 8-bromo-1-(cyclohex-2-en-1-yl)-2-oxo-1,2,3,5-tetrahydro-4H-pyrido[3,2-e][1,4]diazepine-4-carboxylate (220 mg, 0.52 mmol) in cyclohexane (8 mL) and EtOAc (2 mL). The reaction mixture was placed under H₂ atmosphere using a balloon for 90 minutes. The product mixture was filtered through celite and concentrated under reduced pressure. The residue obtained was purified by silica gel column chromatography (0-30% EtOAc/Heptane) to yield the title compound (162 mg, 73% yield). LC-MS calc. for C₁₉H₂₇BrN₃O₃[M+H]⁺: m/z=424.1/426.1; Found: 424.3/426.3.

### Step 3. 6-((8-(4-((5-Acetyl-2-(cyclopropanecarboxamido)pyridin-4-yl)amino)-3-methoxypyridin-2-yl)-1-cyclohexyl-2-oxo-1,2,3,5-tetrahydro-4H-pyrido[3,2-e][1,4]diazepin-4-yl)methyl)-N,N-dimethylpicolinamide

The title compound was prepared in accordance with the synthetic protocols set forth in Example 176 Steps 6-8 using appropriate intermediates, as well as commercial starting materials. LCMS calc. for C₄₀H₄₆N₉O₅ [M+H]⁺: m/z=732.4; Found: 732.5.

### Example 192. 6-((8-(3-((5-Acetyl-2-(cyclopropanecarboxamido)pyridin-4-yl)amino)-2-methoxyphenyl)-2-oxo-1-(tetrahydro-2H-pyran-4-yl)-1,2,3,5-tetrahydro-4H-benzo[e][1,4]diazepin-4-yl)methyl)-N,N-dimethylpicolinamide

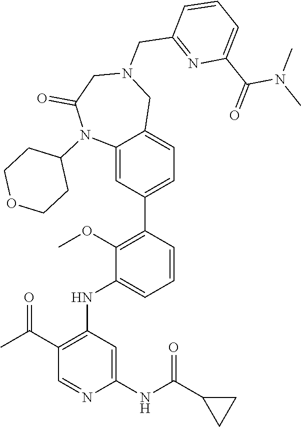 [SMILES: COc1c(Nc2cc(NC(=O)C3CC3)ncc2C(C)=O)cccc1-c1ccc2c(c1)N(C1CCOCC1)C(=O)CN(Cc1cccc(C(=O)N(C)C)n1)C2]

The title compound was prepared in accordance with the synthetic protocols set forth in Example 190 Steps 1 through 7 using appropriate intermediates, as well as commercial starting materials. LCMS calc. for C₄₁H₄₆N₇O₆ [M+H]⁺: m/z=732.4; Found: 732.5.

### Example 193. 6-((8-(3-((5-Acetyl-2-(cyclopropanecarboxamido)pyridin-4-yl)amino)-5-fluoro-2-methoxyphenyl)-1-(2-hydroxy-2-methylpropyl)-2-oxo-1,2,3,5-tetrahydro-4H-benzo[e][1,4]diazepin-4-yl)methyl)-N,N-dimethylpicolinamide

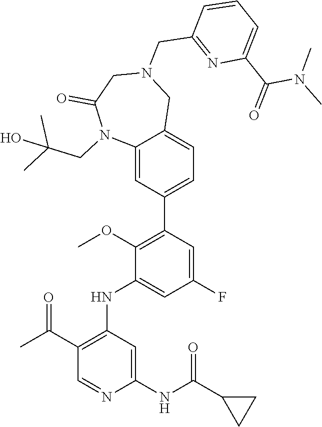 [SMILES: COc1c(Nc2cc(NC(=O)C3CC3)ncc2C(C)=O)cc(F)cc1-c1ccc2c(c1)N(CC(C)(C)O)C(=O)CN(Cc1cccc(C(=O)N(C)C)n1)C2]

### Step 1. tert-Butyl 8-bromo-1-(2-hydroxy-2-methylpropyl)-2-oxo-1,2,3,5-tetrahydro-4H-benzo[e][1,4]diazepine-4-carboxylate

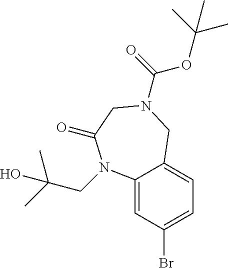 [SMILES: CC(C)(O)CN1C(=O)CN(C(=O)OC(C)(C)C)Cc2ccc(Br)cc21]

Cesium carbonate (955 mg, 2.93 mmol) was added to a stirring solution of tert-butyl 8-bromo-2-oxo-1,2,3,5-tetrahydro-4H-benzo[e][1,4]diazepine-4-carboxylate (500 mg, 1.47 mmol) and 2,2-dimethyloxirane (211 mg, 2.93 mmol) in DMF (5 mL). The reaction mixture was heated to 50° C. and stirred for 15 hours. The product mixture was diluted with water (50 mL) and extracted with EtOAc (3×50 mL). The combined organic layers were dried over Na₂SO₄, filtered, and concentrated under reduced pressure. The residue obtained was purified by silica gel column chromatography (5-50% EtOAc/Hexanes) to yield the title compound (342 mg, 56% yield). LCMS calc. for C₁₈H₂₅BrN₂NaO₄ [M+Na]⁺: m/z=435.1/417.1; Found: 435.1/437.0.

### Step 2. 6-((8-Bromo-1-(2-hydroxy-2-methylpropyl)-2-oxo-1,2,3,5-tetrahydro-4H-benzo[e][1,4]diazepin-4-yl)methyl)-N,N-dimethylpicolinamide

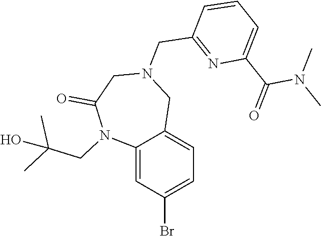 [SMILES: CN(C)C(=O)c1cccc(CN2CC(=O)N(CC(C)(C)O)c3cc(Br)ccc3C2)n1]

The title compound was prepared in accordance with the synthetic protocols set forth in Example 176 Steps 6 using appropriate intermediates, as well as commercial starting materials. LCMS calc. for C₂₂H₂₈BrN₄O₃[M+H]⁺: m/z=475.1/477.1; Found: 475.1/477.1.

### Step 3. 6-((8-(3-((5-Acetyl-2-(cyclopropanecarboxamido)pyridin-4-yl)amino)-5-fluoro-2-methoxyphenyl)-1-(2-hydroxy-2-methylpropyl)-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 84 Steps 1 and 2 using appropriate intermediates, as well as commercial starting materials. LCMS calc. for C₄₀H₄₅FN₇O₆[M+H]⁺: m/z=738.3; Found: 738.4. ¹H NMR (600 MHz, DMSO-d₆) δ 11.18 (s, 1H), 11.07 (s, 1H), 8.88 (s, 1H), 8.11 (s, 1H), 8.02 (s, 1H), 7.85 (s, 1H), 7.64 (d, J=8.0 Hz, 1H), 7.56 (s, 3H), 7.43-7.38 (m, 1H), 7.13 (dd, J=9.0, 2.8 Hz, 1H), 4.33 (d, J=49.2 Hz, 6H), 3.47 (s, 2H), 3.34 (s, 3H), 3.02 (s, 3H), 2.94 (s, 3H), 2.66 (s, 3H), 2.08-2.01 (m, 1H), 1.04 (s, 6H), 0.83 (d, J=7.9 Hz, 4H).

### Example 194. 6-((8-(3-((5-Acetyl-2-(cyclopropanecarboxamido)pyridin-4-yl)amino)-2-methoxyphenyl)-1-cyclopropyl-2-oxo-1,2,3,5-tetrahydro-4H-benzo[e][1,4]diazepin-4-yl)methyl)-N,N-dimethylpicolinamide

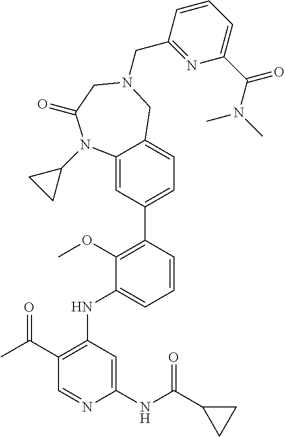 [SMILES: COc1c(Nc2cc(NC(=O)C3CC3)ncc2C(C)=O)cccc1-c1ccc2c(c1)N(C1CC1)C(=O)CN(Cc1cccc(C(=O)N(C)C)n1)C2]

### Step 1. 6-((1-Cyclopropyl-2-oxo-8-(4,4,5,5-tetramethyl-1, 3,2-dioxaborolan-2-yl)-1,2,3,5-tetrahydro-4H-benzo[e][1,4]diazepin-4-yl)methyl)-N,N-dimethylpicolinamide

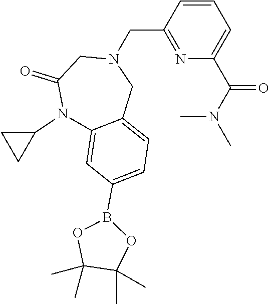 [SMILES: CN(C)C(=O)c1cccc(CN2CC(=O)N(C3CC3)c3cc(B4OC(C)(C)C(C)(C)O4)ccc3C2)n1]

The title compound was synthesized by a procedure analogous to that outlined in Example 14 Step 1 with appropriate starting materials. LCMS calc. for C₂₇H₃₆BN₄O₄[M+H]⁺: m/z=491.3; Found: 491.4.

### Step 2. 6-((8-(3-((5-Acetyl-2-(cyclopropanecarboxamido)pyridin-4-yl)amino)-2-methoxyphenyl)-1-cyclopropyl-2-oxo-1,2,3,5-tetrahydro-4H-benzo[e][1,4]diazepin-4-yl)methyl)-N,N-dimethylpicolinamide

The title compound, as the formate salt, was synthesized by a procedure analogous to that outlined in Example 14 Step 2 with appropriate starting materials. LCMS calc. for C₃₉H₄₂N₇O₅ [M+H]⁺: m/z=688.3; Found: 688.6.

### Example 195. 3-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-2-methoxy-N,N-dimethylbenzamide

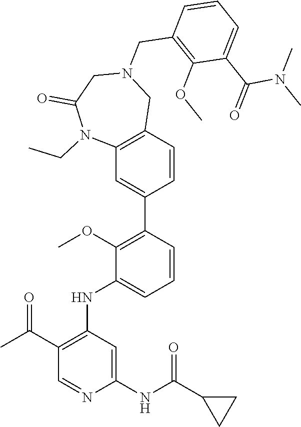 [SMILES: CCN1C(=O)CN(Cc2cccc(C(=O)N(C)C)c2OC)Cc2ccc(-c3cccc(Nc4cc(NC(=O)C5CC5)ncc4C(C)=O)c3OC)cc21]

### Step 1. 3-Formyl-2-methoxy-N,N-dimethylbenzamide

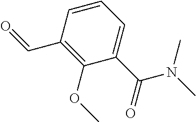

To a mixture of 3-formyl-2-methoxybenzoic acid (29.0 mg, 0.16 mmol), dimethylamine HCl (30.0 mg, 0.37 mmol) and 1-[bis(dimethylamino)methylene]-1H-1,2,3-triazolo[4,5-b]pyridinium 3-oxid hexafluorophosphate (73.45 mg, 0.19 mmol) in DCM (2 mL) was added N,N-diisopropylethylamine (112.15 μL, 0.64 mmol) dropwise at 0° C. The mixture was stirred at rt for 1 h. The mixture was quenched with water, concentrated and purified on FCC (0-100% EtOAc/Hex) to give the title compound (27 mg, 0.1303 mmol, 81% yield). LCMS calc. for C₁₁H₁₄NO₃ [M+H]⁺: m/z=208.1; Found: 208.2.

### Step 2. 3-((8-Bromo-2-oxo-1,2,3,5-tetrahydro-4H-benzo[e][1,4]diazepin-4-yl)methyl)-2-methoxy-N,N-dimethylbenzamide

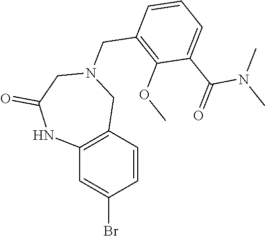 [SMILES: COc1c(CN2CC(=O)Nc3cc(Br)ccc3C2)cccc1C(=O)N(C)C]

A mixture of 8-bromo-1,3,4,5-tetrahydro-1,4-benzodiazepin-2-one; 2,2,2-trifluoroacetic acid (TFA salt of Int. 25) (46.5 mg, 0.13 mmol), 3-formyl-2-methoxybenzoic acid (27.14 mg, 0.13 mmol), N,N-diisopropylethylamine (22.81 μL, 0.13 mmol) and acetic acid (11.23 μL, 0.2 mmol) in DMF (2 mL) was stirred at rt for 30 minutes then sodium triacetoxyborohydride (83.26 mg, 0.39 mmol) was added at 0° C. The mixture was stirred at rt overnight. The mixture was quenched with water, extracted with DCM and purified on FCC (0-100% EtOAc/Hex) to give the title compound (8.3 mg, 0.019 mmol, 15% yield). LCMS calc. for C₂₀H₂₃BrN₃O₃[M+H]⁺: m/z=432.1; Found: 432.1.

### Step 3. 3-((8-Bromo-1-ethyl-2-oxo-1,2,3,5-tetrahydro-4H-benzo[e][1,4]diazepin-4-yl)methyl)-2-methoxy-N,N-dimethylbenzamide

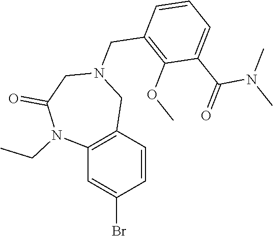 [SMILES: CCN1C(=O)CN(Cc2cccc(C(=O)N(C)C)c2OC)Cc2ccc(Br)cc21]

A mixture of 3-((8-bromo-2-oxo-1,2,3,5-tetrahydro-4H-benzo[e][1,4]diazepin-4-yl)methyl)-2-methoxy-N,N-dimethylbenzamide (8.3 mg, 0.02 mmol), cesium carbonate (12.5 mg, 0.04 mmol) and iodoethane (2.3 μL, 0.03 mmol) in DMSO (1 mL) was stirred at rt for 1 h. The mixture was purified on prepLCMS (pH 2) to obtain the title compound (6.7 mg, 0.0145 mmol, 76% yield). LCMS calc. for C₂₂H₂₇BrN₃O₃[M+H]⁺: m/z=460.1; Found: 460.2.

### Step 4. (4-(3-(Dimethylcarbamoyl)-2-methoxybenzyl)-1-ethyl-2-oxo-2,3,4,5-tetrahydro-1H-benzo[e][1,4]diazepin-8-yl)boronic acid

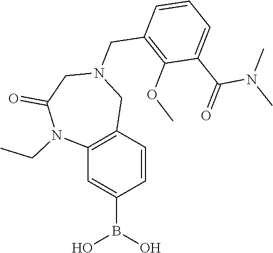 [SMILES: CCN1C(=O)CN(Cc2cccc(C(=O)N(C)C)c2OC)Cc2ccc(B(O)O)cc21]

A mixture of 3-((8-bromo-1-ethyl-2-oxo-1,2,3,5-tetrahydro-4H-benzo[e][1,4]diazepin-4-yl)methyl)-2-methoxy-N,N-dimethylbenzamide (6.7 mg, 10 μmol), bis(pinacolato)diboron (7.39 mg, 30 μmol), [1,1′-bis(diphenylphosphino)ferrocene]dichloropalladium (II) complex with dichloromethane (1.79 mg, 2.2 μmol) and potassium pivalate (6.12 mg, 40 μmol) in 1,4-dioxane (2 mL) was sparged with N₂ for −1 min then stirred at 90° C. for 2 h. The mixture was filtered to give the title compound (6 mg, 0.014 mmol, 97% yield) as crude. LCMS calc. for C₂₂H₂₉BN₃O₅[M+H]⁺: m/z=426.2; Found: 426.2.

### Step 5. 3-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-2-methoxy-N,N-dimethylbenzamide

A mixture of (4-(3-(dimethylcarbamoyl)-2-methoxybenzyl)-1-ethyl-2-oxo-2,3,4,5-tetrahydro-1H-benzo[e][1,4]diazepin-8-yl)boronic acid (6.0 mg, 0.01 mmol), N-(5-acetyl-4-((3-bromo-2-methoxyphenyl)amino)pyridin-2-yl)cyclopropanecarboxamide (Int. 3) (8.0 mg, 0.02 mmol), potassium carbonate (3.9 mg, 0.03 mmol) and XPhos-Pd-G2 (1.67 mg, 2.1 μmol) in 1,4-dioxane (1 mL) and water (0.10 mL) was purged with N₂ and then stirred at 100° C. for 2 h. The mixture was purified on prep LCMS to give the TFA salt of the title compound (3.8 mg, 38.2% yield). LCMS calc. for C₄₀H₄₅N₆O₆ [M+H]⁺: m/z=705.3; Found: 705.4.

### Example 196

Example 196 is set forth in Table 20 and was prepared in accordance with the synthetic protocols set forth in Example 195 using the appropriate intermediates, as well as commercial starting materials. The following compounds is a TFA salt.

TABLE 20
Example 196

| | | | Analytical Data (LCMS |
|---|---|---|---|
| Ex. | Name | Structure | and/or NMR) |
| 196 | 3-((8-(3-((5-acetyl-2- (cyclopropanecarboxamido) pyridin-4-yl)amino)-2- methoxyphenyl)-1-ethyl-2- oxo-1,2,3,5-tetrahydro-4H- benzo[e][1,4]diazepin-4- yl)methyl)-N,N,2- trimethylbenzamide | 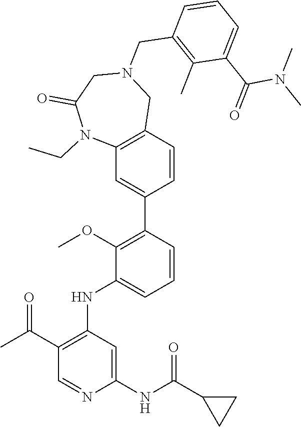 [SMILES: CCN1C(=O)CN(Cc2cccc(C(=O)N(C)C)c2C)Cc2ccc(-c3cccc(Nc4cc(NC(=O)C5CC5)ncc4C(C)=O)c3OC)cc21] | LCMS calc. for C₄₀H₄₅N₆O₅ [M + H]⁺: m/z = 689.3; Found: 689.2. |

### Example 197. 6-(8-(3-((5-Acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylpicolinamide

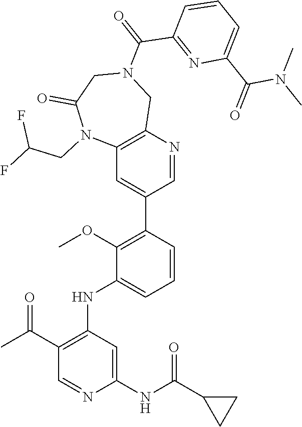 [SMILES: COc1c(Nc2cc(NC(=O)C3CC3)ncc2C(C)=O)cccc1-c1cnc2c(c1)N(CC(F)F)C(=O)CN(C(=O)c1cccc(C(=O)N(C)C)n1)C2]

### Step 1. Methyl 6-(dimethylcarbamoyl)picolinate

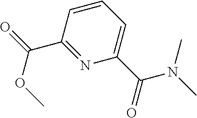

To a mixture of 6-(methoxycarbonyl)picolinic acid (35.0 mg, 0.19 mmol), dimethylamine hydrochloride (23.63 mg, 0.29 mmol) and 1-[bis(dimethylamino)methylene]-1H-1,2,3-triazolo[4,5-b]pyridinium 3-oxid hexafluorophosphate (80.81 mg, 0.21 mmol) in DCM (2 mL) was added N,N-diisopropylethylamine (100.96 μL, 0.58 mmol). The mixture was stirred at rt for 1 h. The mixture was washed with water, dried and concentrated to give methyl 6-(dimethylcarbamoyl)picolinate (40 mg, 0.19 mmol, 99.4% yield) as crude. LCMS calc. for C₁₀H₁₃N₂O₃ [M+H]⁺: m/z=209.1; Found: 209.2.

### Step 2. 6-(Dimethylcarbamoyl)picolinic acid

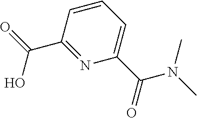

Methyl 6-(dimethylcarbamoyl)picolinate (40.0 mg, 0.19 mmol) was treated with lithium hydroxide (46.01 mg, 1.92 mmol) in THF (2 mL) and Water (2 mL) at rt for 30 min. The mixture was purified on prepLCMS to give the title compound (28 mg, 0.14 mmol, 75% yield). LCMS calc. for C₉H₁₁N₂O₃ [M+H]⁺: m/z=195.1; Found: 195.1.

### Step 3. 6-(8-Bromo-1-(2,2-difluoroethyl)-2-oxo-2,3,4,5-tetrahydro-1H-pyrido[3,2-e][1,4]diazepine-4-carbonyl)-N,N-dimethylpicolinamide

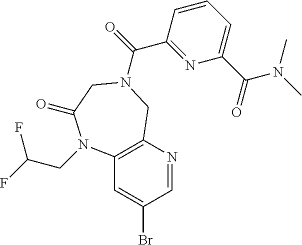 [SMILES: CN(C)C(=O)c1cccc(C(=O)N2CC(=O)N(CC(F)F)c3cc(Br)cnc3C2)n1]

To a mixture of 8-bromo-1-(2,2-difluoroethyl)-1,3,4,5-tetrahydro-2H-pyrido[3,2-e][1,4]-diazepin-2-one; bis(2,2,2-trifluoroacetic acid) (24.8 mg, 0.05 mmol), 6-(dimethylcarbamoyl)-picolinic acid (9.0 mg, 0.05 mmol) and 1-[bis(dimethylamino)methylene]-1H-1,2,3-triazolo[4,5-b]pyridinium 3-oxid hexafluorophosphate (21.2 mg, 0.06 mmol) in DCM (2 mL) was added N,N-diisopropylethylamine (48.4 μL, 0.28 mmol). The mixture was stirred at rt for 1 h. The mixture was diluted with water and purified on prepLCMS to give the title compound (8.7 mg, 0.018 mmol, 39% yield). LCMS calc. for C₁₉H₁₉BrF₂N₅O₃ [M+H]⁺: m/z=482.1; Found: 482.2.

### Step 4. 6-(8-(3-((5-Acetyl-2-(cyclopropanecarboxamido)pyridin-4-yl)amino)-2-methoxy-phenyl)-1-(2,2-difluoroethyl)-2-oxo-2,3,4,5-tetrahydro-1H-pyrido[3,2-e][1,4]diazepine-4-carbonyl)-N,N-dimethylpicolinamide

A mixture of 6-(8-bromo-1-(2,2-difluoroethyl)-2-oxo-2,3,4,5-tetrahydro-1H-pyrido[3,2-e][1,4]diazepine-4-carbonyl)-N,N-dimethylpicolinamide (8.7 mg, 0.02 mmol), [3-[[5-acetyl-2-(cyclopropanecarbonylamino)-4-pyridinyl]amino]-2-methoxyphenyl]boronic acid (8.66 mg, 0.02 mmol), [1,1′-bis(diphenylphosphino)ferrocene]dichloropalladium (II) complex with dichloromethane (2.22 mg, 0. mmol) and potassium carbonate (9.97 mg, 0.07 mmol) in 1,4-dioxane (1 mL) and water (0.10 mL) was degassed and then stirred at 90° C. for 2 h. The mixture was purified on prepLCMS to give the title compound (5 mg, 0.0069 mmol, 38% yield) as a TFA salt. LCMS calc. for C₃₇H₃₇F₂N₈O₆[M+H]⁺: m/z=727.3; Found: 727.4.

### Example 198. 5-(8-(3-((5-Acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylbicyclo[3.1.1]heptane-1-carboxamide

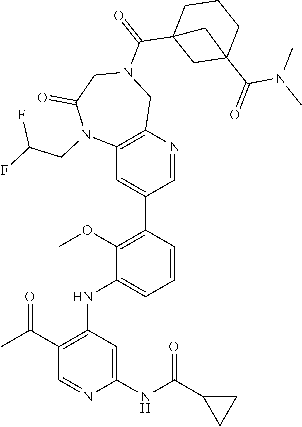 [SMILES: COc1c(Nc2cc(NC(=O)C3CC3)ncc2C(C)=O)cccc1-c1cnc2c(c1)N(CC(F)F)C(=O)CN(C(=O)C13CCCC(C(=O)N(C)C)(C1)C3)C2]

The title compound was prepared in accordance with the synthetic protocols set forth in Example 197 Steps 2 through 4 using appropriate intermediates, as well as commercial starting materials. LCMS calc. for C₃₉H₄₄F₂N₇O₆[M+H]⁺: m/z=744.3; Found: 744.4.

### Example 199. 6-((1-(4-(1H-Pyrazol-1-yl)phenyl)-8-(3-((5-acetyl-2-(cyclopropanecarboxamido)-pyridin-4-yl)amino)-2-methoxyphenyl)-2-oxo-1,2,3,5-tetrahydro-4H-benzo[e][1,4]diazepin-4-yl)methyl)-N,N-dimethylpicolinamide

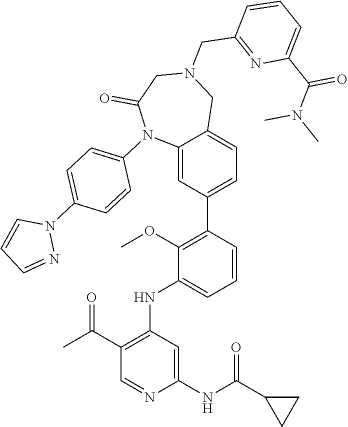 [SMILES: COc1c(Nc2cc(NC(=O)C3CC3)ncc2C(C)=O)cccc1-c1ccc2c(c1)N(c1ccc(-n3cccn3)cc1)C(=O)CN(Cc1cccc(C(=O)N(C)C)n1)C2]

### Step 1. 6-((1-(4-(1H-Pyrazol-1-yl)phenyl)-8-bromo-2-oxo-1,2,3,5-tetrahydro-4H-benzo[e][1,4]diazepin-4-yl)methyl)-N,N-dimethylpicolinamide and 6-((1-(4-(1H-pyrazol-1-yl)phenyl)-8-iodo-2-oxo-1,2,3,5-tetrahydro-4H-benzo[e][1,4]diazepin-4-yl)methyl)-N,N-dimethylpicolinamide

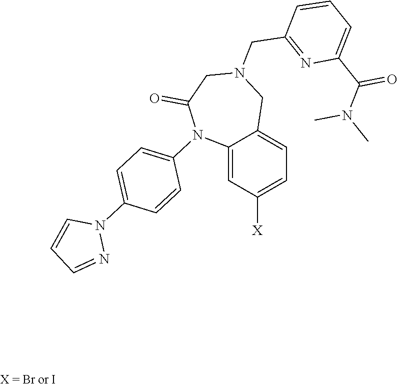

Potassium carbonate (102.8 mg, 0.74 mmol), copper iodide (9.5 mg, 0.05 mmol), 6-((8-bromo-2-oxo-1,2,3,5-tetrahydro-4H-benzo[e][1,4]diazepin-4-yl)methyl)-N,N-dimethylpicolinamide (Int. 26) (100.0 mg, 0.25 mmol), 1-(4-iodophenyl)pyrazole (167.4 mg, 0.62 mmol) and (+\−)-trans-1,2-diaminocyclohexane (5.6 mg, 0.05 mmol) were added in 1,4-dioxane (2 mL) under N₂. It stirred at 125° C. overnight. The reaction was filtered. Water was added and it was extracted with EtOAc (3×10 mL). The organic portion was concentrated and purified by silica gel column chromatography (10-40% EtOAc/Heptanes) to give the title compound (70 mg, 0.13 mmol, 52% yield). This reaction brings some Br/I exchange compound. LCMS calc. for C₂₇H₂₆BrN₆O₂[M+H]⁺: m/z=545.1/547.1; Found: 545.0/546.9. LCMS calc. for C₂₇H₂₆IN₆O₂[M+H]⁺: m/z=593.1; Found: 593.7.

### Step 2. 6-((1-(4-(1H-Pyrazol-1-yl)phenyl)-8-(3-((5-acetyl-2-(cyclopropanecarboxamido-)pyridin-4-yl)amino)-2-methoxyphenyl)-2-oxo-1,2,3,5-tetrahydro-4H-benzo[e][1,4]diazepin-4-yl)methyl)-N,N-dimethylpicolinamide

[3-[[5-Acetyl-2-(cyclopropanecarbonylamino)-4-pyridinyl]amino]-2-methoxyphenyl]-boronic acid (9.4 mg, 0.03 mmol), [1,1′-bis(diphenylphosphino)ferrocene]dichloropalladium (II) complex with dichloromethane (2.1 mg, 0.003 mmol), cesium acetate (24.3 mg, 0.13 mmol) and 6-((1-(4-(1H-pyrazol-1-yl)phenyl)-8-iodo-2-oxo-1,2,3,5-tetrahydro-4H-benzo[e][1,4]diazepin-4-yl)methyl)-N,N-dimethylpicolinamide (15.0 mg, 0.03 mmol) were added in 1,4-dioxane (0.95 mL) and water (50 μL) under N₂. It was stirred at 95° C. overnight. Reaction was quenched with water and extracted with EtOAc (3×5 mL). The organic phase was concentrated and purified by prep-HPLC (15-32% MeCN in 0.05% formic acid (aq)) to give the formate salt of the title compound (16.2 mg, 0.021 mmol, 81% yield) as a white solid. LCMS calc. for C₄₅H₄₄N₉O₅ [M+H]⁺: m/z=790.35; Found: 790.67. ¹H NMR (300 MHz, Methanol-d₄) δ 8.70 (s, 1H), 8.26 (d, J=2.3 Hz, 1H), 8.05 (t, J=7.7 Hz, 1H), 7.85 (d, J=8.9 Hz, 1H), 7.78-7.66 (m, 2H), 7.62 (d, J=7.8 Hz, 2H), 7.52-7.41 (m, 3H), 7.37 (t, J=6.5 Hz, 1H), 7.31 (d, J=8.2 Hz, 1H), 6.66 (s, 1H), 6.55 (s, 1H), 4.62-4.31 (m, 6H), 3.89-3.71 (m, 4H), 3.15 (s, 3H), 3.06 (s, 3H), 2.83 (bs, 2H), 2.65 (s, 3H), 1.74 (s, 1H), 1.14-0.92 (m, 4H).

### Example 200. 6-((1-(6-(1H-Pyrazol-1-yl)pyridin-3-yl)-8-(3-((5-acetyl-2-(cyclopropane-carboxamido)pyridin-4-yl)amino)-2-methoxyphenyl)-2-oxo-1,2,3,5-tetrahydro-4H-benzo[e][1,4]diazepin-4-yl)methyl)-N,N-dimethylpicolinamide

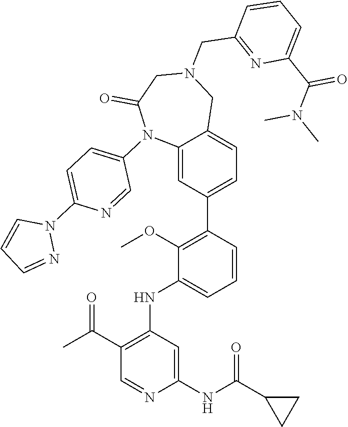 [SMILES: COc1c(Nc2cc(NC(=O)C3CC3)ncc2C(C)=O)cccc1-c1ccc2c(c1)N(c1ccc(-n3cccn3)nc1)C(=O)CN(Cc1cccc(C(=O)N(C)C)n1)C2]

The title compound was prepared in accordance with the synthetic protocols set forth in Example 199 using appropriate intermediates, as well as commercial starting materials. LCMS calc. for C₄₄H₄₃N₁₀O₅[M+H]⁺: m/z=791.3; Found: 791.9. ¹H NMR (300 MHz, Methanol-d₄) δ 8.69 (s, 1H), 8.58 (d, J=2.6 Hz, 1H), 8.46 (d, J=2.4 Hz, 1H), 8.15-7.93 (m, 3H), 7.84-7.60 (m, 5H), 7.53-7.33 (m, 4H), 6.67-6.51 (m, 2H), 4.66 (s, 4H), 3.97 (s, 2H), 3.35 (s, 3H), 3.16 (s, 3H), 3.07 (s, 3H), 2.65 (s, 3H), 1.85-1.69 (m, 1H), 1.16-1.02 (m, 4H).

### Example 201. 6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)methyl)-N,N-dimethylpicolinamide

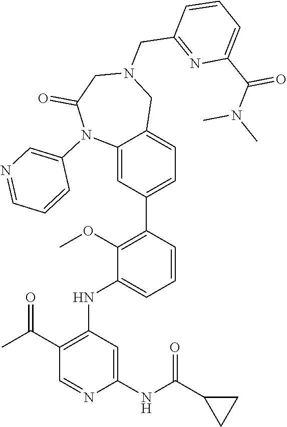 [SMILES: COc1c(Nc2cc(NC(=O)C3CC3)ncc2C(C)=O)cccc1-c1ccc2c(c1)N(c1cccnc1)C(=O)CN(Cc1cccc(C(=O)N(C)C)n1)C2]

### Step 1. 6-((8-Bromo-2-oxo-1-(pyridin-3-yl)-1,2,3,5-tetrahydro-4H-benzo[e][1,4]diazepin-4-yl)methyl)-N,N-dimethylpicolinamide

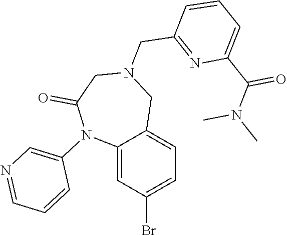 [SMILES: CN(C)C(=O)c1cccc(CN2CC(=O)N(c3cccnc3)c3cc(Br)ccc3C2)n1]

Pyridine 3-boronic acid (15.0 mg, 0.12 mmol), copper acetate (11.0 mg, 0.06 mmol), 6-((8-bromo-2-oxo-1,2,3,5-tetrahydro-4H-benzo[e][1,4]diazepin-4-yl)methyl)-N,N-dimethylpicolinamide (Int. 26) (25.0 mg, 0.06 mmol) and 4-(dimethylamino)pyridine (23.0 mg, 0.19 mmol) were added in toluene (1 mL) under air. The reaction mixture was stirred at 100° C. for overnight. The product mixture was quenched with water and extracted with EtOAc (3×5 mL). The organic portion was concentrated and purified by prep-HPLC (15-35% MeCN in 0.05% TFA (aq)) to give the title compound (12 mg, 0.025 mmol, 40% yield) as a white solid. LCMS calc. for C₂₃H₂₃BrN₅O₂ [M+H]⁺: m/z=480.1/482.1; Found: 480.3/482.3.

### Step 2. 6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)methyl)-N,N-dimethylpicolinamide

A mixture of (3-((5-acetyl-2-(cyclopropanecarboxamido)pyridin-4-yl)amino)-2-methoxy-phenyl)boronic acid (13.3 mg, 0.04 mmol), [1,1′-bis(diphenylphosphino)ferrocene]dichloro-palladium (II) complex with dichloromethane (2.2 mg, 3 μmol), cesium acetate (28.8 mg, 0.15 mmol) and 6-((8-bromo-2-oxo-1-(pyridin-3-yl)-1,2,3,5-tetrahydro-4H-benzo[e][1,4]diazepin-4-yl)methyl)-N,N-dimethylpicolinamide (14.4 mg, 0.03 mmol) was dissolved in water (60 μL) and 1,4-dioxane (0.90 mL) under N₂. It was stirred at 100° C. overnight. The reaction mixture was washed with water and extracted with EtOAc (3×5 mL). The combined organic layers were concentrated and purified by silica chromatography (0-5% MeOH/EtOAc) and prep-HPLC (15-40% MeCN/Water, 0.05% TFA) to give the title compound (7.2 mg, 0.010 mmol, 33% yield) as a white solid. LCMS calc. for C₄₁H₄₁N₈O₅ [M+H]⁺: m/z=725.32; Found: 725.61. ¹H NMR (300 MHz, Methanol-d₄) δ 8.74 (s, 1H), 8.64 (d, J=2.4 Hz, 1H), 8.57 (dd, J=5.0, 1.5 Hz, 1H), 8.06 (t, J=7.8 Hz, 1H), 8.00 (ddd, J=8.3, 2.4, 1.6 Hz, 1H), 7.78-7.60 (m, 5H), 7.49 (dd, J=7.5, 2.0 Hz, 1H), 7.43-7.31 (m, 2H), 7.29 (d, J=1.7 Hz, 1H), 6.71 (s, 1H), 4.58 (s, 4H), 3.90 (s, 2H), 3.15 (s, 3H), 3.06 (s, 3H), 2.68 (s, 3H), 2.66 (s, 2H), 1.89-1.69 (m, 1H), 1.22-0.95 (m, 4H).

### Example 202 to 222

Examples 202-222 are set forth in Table 21 and were prepared in accordance with the synthetic protocols set forth in Example 201 using the appropriate intermediates, as well as commercial starting materials. The following compounds are TFA salts unless otherwise noted.

TABLE 21
Examples 202-222

| | | | Analytical Data (LCMS |
|---|---|---|---|
| Ex. | Name | Structure | and/or NMR) |
| 202 | 6-((8-(3-((5-acetyl-2- (cyclopropanecarboxamido) pyridin-4-yl)amino)-2- methoxyphenyl)-1-(6- methoxypyridin-3-yl)-2-oxo- 1,2,3,5-tetrahydro-4H- benzo[e][1,4]diazepin-4- yl)methyl)-N,N- dimethylpicolinamide | 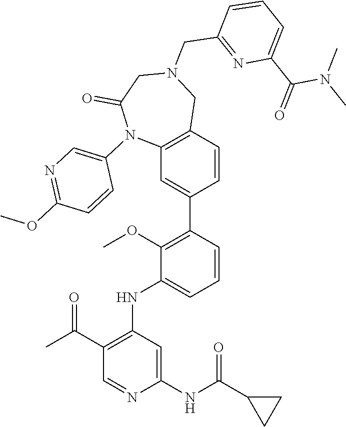 | LCMS calc. for C₄₂H₄₃N₈O₆ [M + H]⁺: m/z = 755.3; Found: 755.2. |
| 203 | 6-((8-(3-((5-acetyl-2- (cyclopropanecarboxamido) pyridin-4-yl)amino)-2- methoxyphenyl)-1-(2- fluorophenyl)-2-oxo-1,2,3,5- tetrahydro-4H- benzo[e][1,4]diazepin-4- yl)methyl)-N,N- dimethylpicolinamide | 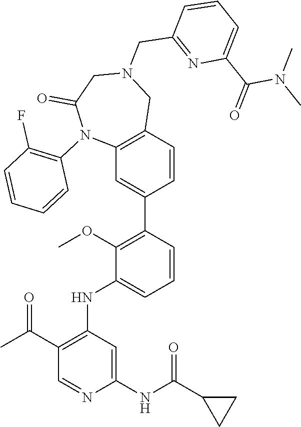 [SMILES: COc1c(Nc2cc(NC(=O)C3CC3)ncc2C(C)=O)cccc1-c1ccc2c(c1)N(c1ccccc1F)C(=O)CN(Cc1cccc(C(=O)N(C)C)n1)C2] | LCMS calc. for C₄₂H₄₁FN₇O₅ [M + H]⁺: m/z = 742.3; Found: 742.2. |
| 204 | 6-((8-(3-((5-acetyl-2- (cyclopropanecarboxamido) pyridin-4-yl)amino)-2- methoxyphenyl)-1-(3,4- difluorophenyl)-2-oxo- 1,2,3,5-tetrahydro-4H- benzo[e][1,4]diazepin-4- yl)methyl)-N,N- dimethylpicolinamide | 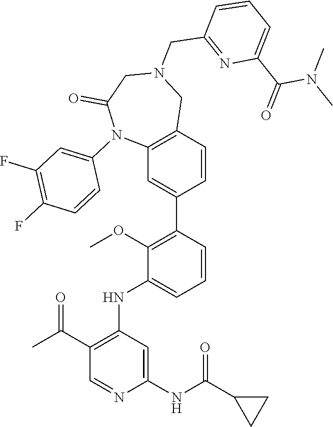 [SMILES: COc1c(Nc2cc(NC(=O)C3CC3)ncc2C(C)=O)cccc1-c1ccc2c(c1)N(c1ccc(F)c(F)c1)C(=O)CN(Cc1cccc(C(=O)N(C)C)n1)C2] | LCMS calc. for C₄₂H₄₀F₂N₇O₅ [M + H]⁺: m/z = 760.3; Found: 760.2. |
| 205 | 6-((8-(3-((5-acetyl-2- (cyclopropanecarboxamido) pyridin-4-yl)amino)-2- methoxyphenyl)-2-oxo-1- (quinolin-7-yl)-1,2,3,5- tetrahydro-4H- benzo[e][1,4]diazepin-4- yl)methyl)-N,N- dimethylpicolinamide | 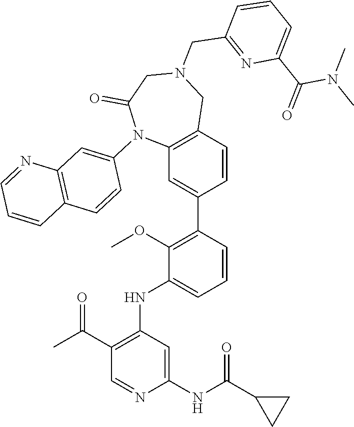 [SMILES: COc1c(Nc2cc(NC(=O)C3CC3)ncc2C(C)=O)cccc1-c1ccc2c(c1)N(c1ccc3cccnc3c1)C(=O)CN(Cc1cccc(C(=O)N(C)C)n1)C2] | LCMS calc. for C₄₅H₄₃N₈O₅ [M + H]⁺: m/z = 775.3; Found: 775.2. |
| 206 | 6-((8-(3-((5-acetyl-2- (cyclopropanecarboxamido) pyridin-4-yl)amino)-2- methoxyphenyl)-2-oxo-1- (quinolin-6-yl)-1,2,3,5- tetrahydro-4H- benzo[e][1,4]diazepin-4- yl)methyl)-N,N- dimethylpicolinamide | 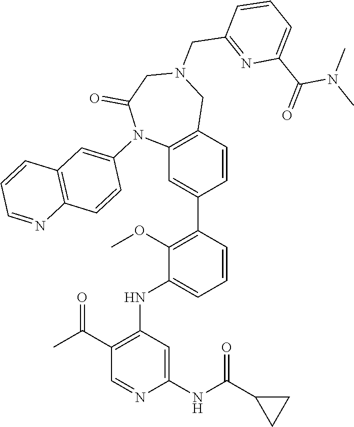 [SMILES: COc1c(Nc2cc(NC(=O)C3CC3)ncc2C(C)=O)cccc1-c1ccc2c(c1)N(c1ccc3ncccc3c1)C(=O)CN(Cc1cccc(C(=O)N(C)C)n1)C2] | LCMS calc. for C₄₅H₄₃N₈O₅ [M + H]⁺: m/z = 775.3; Found: 775.2. |
| 207 | 6-((8-(3-((5-acetyl-2- (cyclopropanecarboxamido) pyridin-4-yl)amino)-2- methoxyphenyl)-1-(5- fluoropyridin-2-yl)-2-oxo- 1,2,3,5-tetrahydro-4H- benzo[e][1,4]diazepin-4- yl)methyl)-N,N- dimethylpicolinamide | 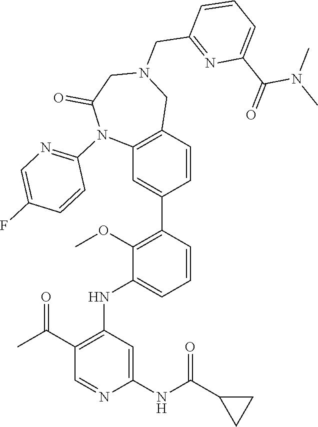 [SMILES: COc1c(Nc2cc(NC(=O)C3CC3)ncc2C(C)=O)cccc1-c1ccc2c(c1)N(c1ccc(F)cn1)C(=O)CN(Cc1cccc(C(=O)N(C)C)n1)C2] | LCMS calc. for C₄₁H₄₀FN₈O₅ [M + H]⁺: m/z = 743.3; Found: 743.2. |
| 208 | 6-((8-(3-((5-acetyl-2- (cyclopropanecarboxamido) pyridin-4-yl)amino)-2- methoxyphenyl)-1-(3- fluoropyridin-4-yl)-2-oxo- 1,2,3,5-tetrahydro-4H- benzo[e][1,4]diazepin-4- yl)methyl)-N,N- dimethylpicolinamide | 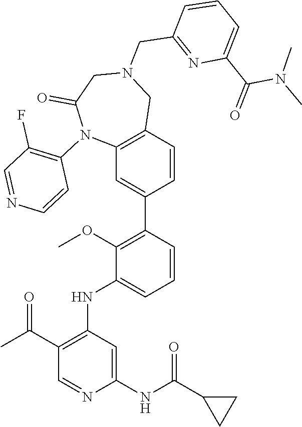 [SMILES: COc1c(Nc2cc(NC(=O)C3CC3)ncc2C(C)=O)cccc1-c1ccc2c(c1)N(c1ccncc1F)C(=O)CN(Cc1cccc(C(=O)N(C)C)n1)C2] | LCMS calc. for C₄₁H₄₀FN₈O₅ [M + H]⁺: m/z = 743.3; Found: 743.2. |
| 209 | 6-((8-(3-((5-acetyl-2- (cyclopropanecarboxamido) pyridin-4-yl)amino)-2- methoxyphenyl)-1-(2,4- difluorophenyl)-2-oxo- 1,2,3,5-tetrahydro-4H- benzo[e][1,4]diazepin-4- yl)methyl)-N,N- dimethylpicolinamide | 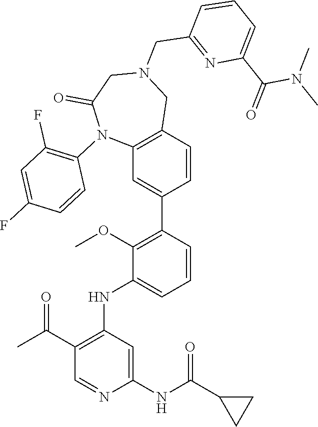 [SMILES: COc1c(Nc2cc(NC(=O)C3CC3)ncc2C(C)=O)cccc1-c1ccc2c(c1)N(c1ccc(F)cc1F)C(=O)CN(Cc1cccc(C(=O)N(C)C)n1)C2] | LCMS calc. for C₄₂H₄₀F₂N₇O₅ [M + H]⁺: m/z = 760.3; Found: 760.2. |
| 210 | 6-((8-(3-((5-acetyl-2- (cyclopropanecarboxamido) pyridin-4-yl)amino)-2- methoxyphenyl)-1-(4- cyanophenyl)-2-oxo-1,2,3,5- tetrahydro-4H- benzo[e][1,4]diazepin-4- yl)methyl)-N,N- dimethylpicolinamide | 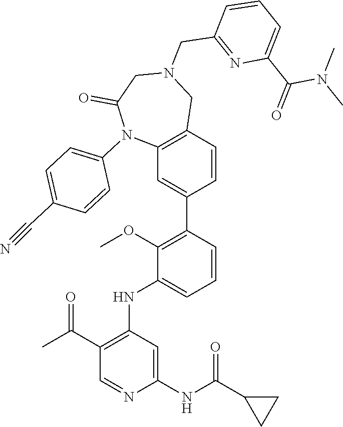 [SMILES: COc1c(Nc2cc(NC(=O)C3CC3)ncc2C(C)=O)cccc1-c1ccc2c(c1)N(c1ccc(C#N)cc1)C(=O)CN(Cc1cccc(C(=O)N(C)C)n1)C2] | LCMS calc. for C₄₃H₄₁N₈O₅ [M + H]⁺: m/z = 749.3; Found: 749.2. |
| 211 | 6-((8-(3-((5-acetyl-2- (cyclopropanecarboxamido) pyridin-4-yl)amino)-2- methoxyphenyl)-2-oxo-1- phenyl-1,2,3,5-tetrahydro- 4H-benzo[e][1,4]diazepin-4- yl)methyl)-N,N- dimethylpicolinamide | 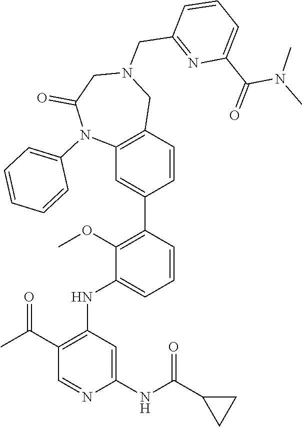 [SMILES: COc1c(Nc2cc(NC(=O)C3CC3)ncc2C(C)=O)cccc1-c1ccc2c(c1)N(c1ccccc1)C(=O)CN(Cc1cccc(C(=O)N(C)C)n1)C2] | LCMS calc. for C₄₂H₄₂N₇O₅ [M + H]⁺: m/z = 724.3; Found: 724.2. |
| 212 | 6-((8-(3-((5-acetyl-2- (cyclopropanecarboxamido) pyridin-4-yl)amino)-2- methoxyphenyl)-1-(3- chlorophenyl)-2-oxo-1,2,3,5- tetrahydro-4H- benzo[e][1,4]diazepin-4- yl)methyl)-N,N- dimethylpicolinamide | 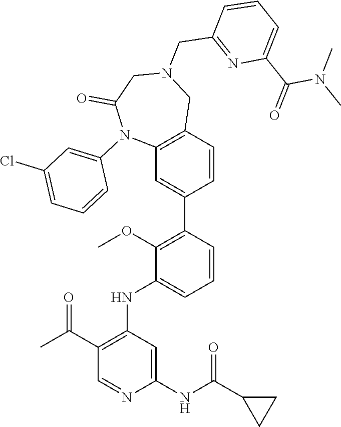 [SMILES: COc1c(Nc2cc(NC(=O)C3CC3)ncc2C(C)=O)cccc1-c1ccc2c(c1)N(c1cccc(Cl)c1)C(=O)CN(Cc1cccc(C(=O)N(C)C)n1)C2] | LCMS calc. for C₄₂H₄₁ClN₇O₅ [M + H]⁺: m/z = 758.3; Found: 758.2. |
| 213 | 6-((8-(3-((5-acetyl-2- (cyclopropanecarboxamido) pyridin-4-yl)amino)-2- methoxyphenyl)-1-(3- fluorophenyl)-2-oxo-1,2,3,5- tetrahydro-4HI- benzo[e][1,4]diazepin-4- yl)methyl)-N,N- dimethylpicolinamide | 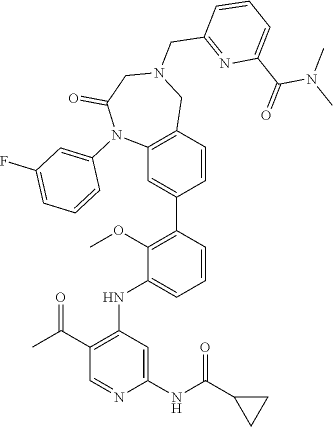 [SMILES: COc1c(Nc2cc(NC(=O)C3CC3)ncc2C(C)=O)cccc1-c1ccc2c(c1)N(c1cccc(F)c1)C(=O)CN(Cc1cccc(C(=O)N(C)C)n1)C2] | LCMS calc. for C₄₂H₄₁FN₇O₅ [M + H]⁺: m/z = 742.3; Found: 742.2. |
| 214 | 6-((8-(3-((5-acetyl-2- (cyclopropanecarboxamido) pyridin-4-yl)amino)-2- methoxyphenyl)-1-(6- methylpyridin-3-yl)-2-oxo- 1,2,3,5-tetrahydro-4H- benzo[e][1,4]diazepin-4- yl)methyl)-N,N- dimethylpicolinamide | 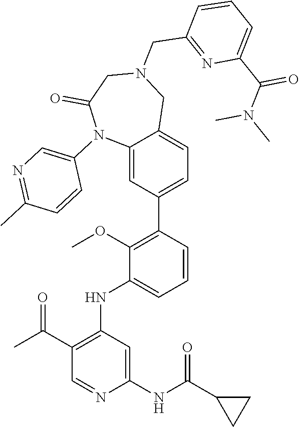 [SMILES: COc1c(Nc2cc(NC(=O)C3CC3)ncc2C(C)=O)cccc1-c1ccc2c(c1)N(c1ccc(C)nc1)C(=O)CN(Cc1cccc(C(=O)N(C)C)n1)C2] | LCMS calc. for C₄₂H₄₃N₈O₅ [M + H]⁺: m/z = 739.3; Found: 739.2. |
| 215 | 6-((8-(4-((5-acetyl-2- (cyclopropanecarboxamido) pyridin-4-yl)amino)-3- methoxypyridin-2-yl)-2-oxo- 1-(quinolin-3-yl)-1,2,3,5- tetrahydro-4H- benzo[e][1,4]diazepin-4- yl)methyl)-N,N- dimethylpicolinamide | 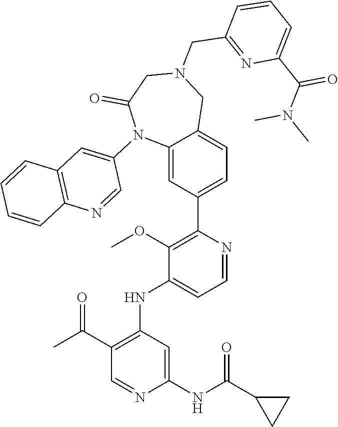 [SMILES: COc1c(Nc2cc(NC(=O)C3CC3)ncc2C(C)=O)ccnc1-c1ccc2c(c1)N(c1cnc3ccccc3c1)C(=O)CN(Cc1cccc(C(=O)N(C)C)n1)C2] | LCMS calc. for C₄₄H₄₂N₉O₅ [M + H]⁺: m/z = 776.3; Found: 776.2. |
| 216 | 6-((8-(3-((5-acetyl-2- (cyclopropanecarboxamido) pyridin-4-yl)amino)-2- methoxyphenyl)-1-(4- fluorophenyl)-2-oxo-1,2,3,5- tetrahydro-4H-pyrido[3,2- e][1,4]diazepin-4-yl)methyl)- N,N-dimethylpicolinamide | 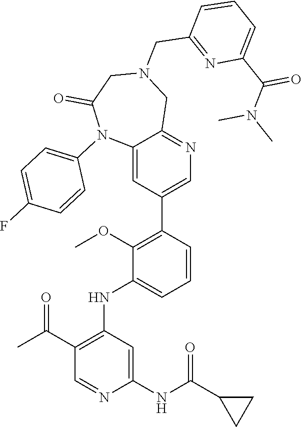 [SMILES: COc1c(Nc2cc(NC(=O)C3CC3)ncc2C(C)=O)cccc1-c1cnc2c(c1)N(c1ccc(F)cc1)C(=O)CN(Cc1cccc(C(=O)N(C)C)n1)C2] | LCMS calc. for C₄₁H₄₀FN₈O₅ [M + H]⁺: m/z = 743.3; Found 743.0. |
| 217 | 6-((8-(3-((5-acetyl-2- (cyclopropanecarboxamido) pyridin-4-yl)amino)-2- methoxyphenyl)-2-oxo-1- (thiazol-5-yl)-1,2,3,5- tetrahydro-4H- benzo[e][1,4]diazepin-4- yl)methyl)-N,N- dimethylpicolinamide | 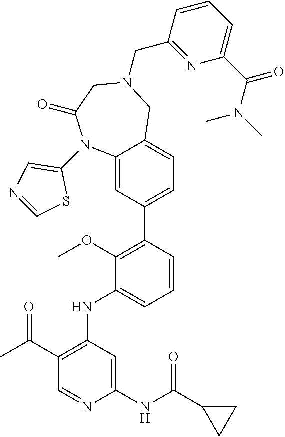 [SMILES: COc1c(Nc2cc(NC(=O)C3CC3)ncc2C(C)=O)cccc1-c1ccc2c(c1)N(c1cncs1)C(=O)CN(Cc1cccc(C(=O)N(C)C)n1)C2] | LCMS calc. for C₃₉H₃₉N₈O₅S [M + H]⁺: m/z = 731.3; Found: 731.0. |
| 218 | 6-((8-(3-((5-acetyl-2- (cyclopropanecarboxamido) pyridin-4-yl)amino)-2- methoxyphenyl)-2-oxo-1- (pyridin-2-yl)-1,2,3,5- tetrahydro-4HI- benzo[e][1,4]diazepin-4- yl)methyl)-N,N- dimethylpicolinamide | 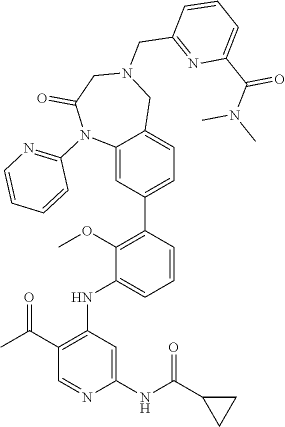 [SMILES: COc1c(Nc2cc(NC(=O)C3CC3)ncc2C(C)=O)cccc1-c1ccc2c(c1)N(c1ccccn1)C(=O)CN(Cc1cccc(C(=O)N(C)C)n1)C2] | LCMS calc. for C₄₁H₄₁N₈O₅ [M + H]⁺: m/z = 725.3; Found: 725.0. |
| 219 | 6-((8-(3-((5-acetyl-2- (cyclopropanecarboxamido) pyridin-4-yl)amino)-2- methoxyphenyl)-2-oxo-1- (pyridin-4-yl)-1,2,3,5- tetrahydro-4H- benzo[e][1,4]diazepin-4- yl)methyl)-N,N- dimethylpicolinamide | 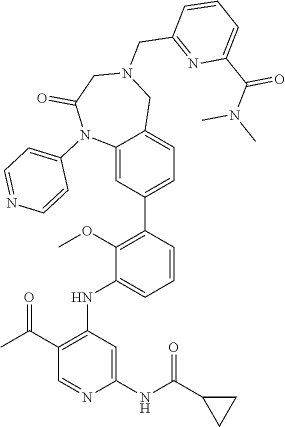 [SMILES: COc1c(Nc2cc(NC(=O)C3CC3)ncc2C(C)=O)cccc1-c1ccc2c(c1)N(c1ccncc1)C(=O)CN(Cc1cccc(C(=O)N(C)C)n1)C2] | LCMS calc. for C₄₁H₄₁N₈O₅ [M + H]⁺: m/z = 725.3; Found: 725.0. |
| 220 | 6-((8-(3-((5-acetyl-2- (cyclopropanecarboxamido) pyridin-4-yl)amino)-2- methoxyphenyl)-1-(5- fluoropyridin-3-yl)-2-oxo- 1,2,3,5-tetrahydro-4H- benzo[e][1,4]diazepin-4- yl)methyl)-N,N- dimethylpicolinamide | 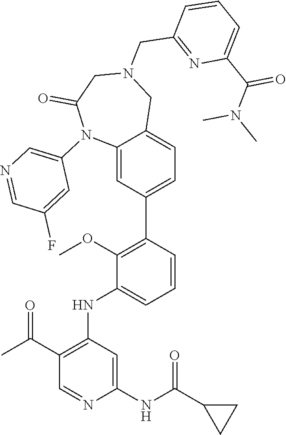 [SMILES: COc1c(Nc2cc(NC(=O)C3CC3)ncc2C(C)=O)cccc1-c1ccc2c(c1)N(c1cncc(F)c1)C(=O)CN(Cc1cccc(C(=O)N(C)C)n1)C2] | LCMS calc. for C₄₁H₄₀FN₈O₅ [M + H]⁺: m/z = 743.3; Found: 743.4. |
| 221 | 6-((8-(3-((5-acetyl-2- (cyclopropanecarboxamido) pyridin-4-yl)amino)-2- methoxyphenyl)-1-(2- methoxypyridin-4-yl)-2-oxo- 1,2,3,5-tetrahydro-4H- benzo[e][1,4]diazepin-4- yl)methyl)-N,N- dimethylpicolinamide | 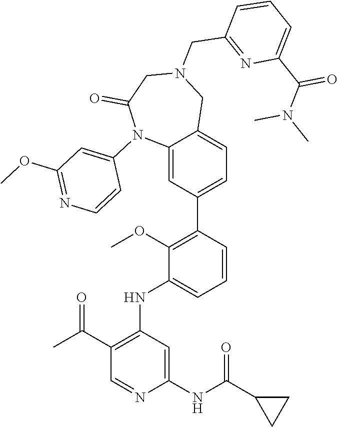 [SMILES: COc1cc(N2C(=O)CN(Cc3cccc(C(=O)N(C)C)n3)Cc3ccc(-c4cccc(Nc5cc(NC(=O)C6CC6)ncc5C(C)=O)c4OC)cc32)ccn1] | LCMS calc. for C₄₂H₄₃N₈O₆ [M + H]⁺: m/z = 755.3; Found: 755.4. |
| 222 | N-(5-acetyl-4-((2-methoxy-3- (4-((6-(morpholine-4- carbonyl)pyridin-2- yl)methyl)-2-oxo-1-(pyridin- 3-yl)-2,3,4,5-tetrahydro-1H- benzo[e][1,4]diazepin-8- yl)phenyl)amino)pyridin-2- yl)cyclopropanecarboxamide | 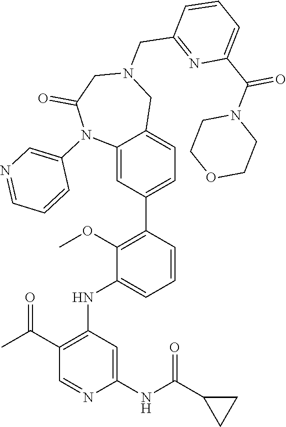 [SMILES: COc1c(Nc2cc(NC(=O)C3CC3)ncc2C(C)=O)cccc1-c1ccc2c(c1)N(c1cccnc1)C(=O)CN(Cc1cccc(C(=O)N3CCOCC3)n1)C2] | LCMS calc. for C₄₃H₄₃N₈O₆ [M + H]⁺ : m/z = 767.33; Found: 767.57. 1H NMR (400 MHZ, DMSO-d₆) δ 11.09 (s, 1H), 10.95 (s, 1H), 8.79 (s, 1H), 8.58 (d, J = 14.2 Hz, 2H), 8.02 (t, J = 7.8 Hz, 1H), 7.87 (dt, J = 8.3, 1.7 Hz, 1H), 7.78 (s, 1H), 7.66-7.54 (m, 5H), 7.43 (dd, J = 7.9, 1.6 Hz, 1H), 7.24 (t, J = 7.8 Hz, 1H), 7.18-7.13 (m, 2H), 4.37 (s, 4H), 3.66 (d, J = 8.7 Hz, 6H), 3.54 (t, J = 4.7 Hz, 2H), 3.40 (t, J = 4.7 Hz, 2H), 3.15 (s, 3H), 2.60 (s, 3H), 1.95 (dt, J = 7.6, 3.0 Hz, 1H), 0.83-0.75 (m, 4H). |

### Example 223. 6-((8-(3-((5-acetyl-2-(cyclopropanecarboxamido)pyridin-4-yl)amino)-2-methoxyphenyl)-1-(methylsulfonyl)-2-oxo-1,2,3,5-tetrahydro-4H-benzo[e][1,4]diazepin-4-yl)methyl)-N,N-dimethylpicolinamide

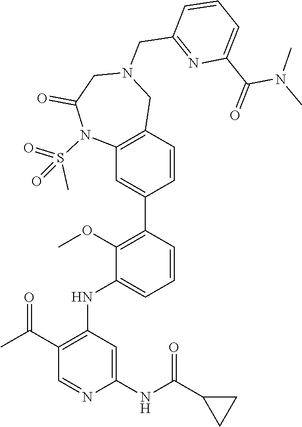 [SMILES: COc1c(Nc2cc(NC(=O)C3CC3)ncc2C(C)=O)cccc1-c1ccc2c(c1)N(S(C)(=O)=O)C(=O)CN(Cc1cccc(C(=O)N(C)C)n1)C2]

### Step 1. 6-((8-bromo-1-(methylsulfonyl)-2-oxo-1,2,3,5-tetrahydro-4H-benzo[e][1,4]-diazepin-4-yl)methyl)-N,N-dimethylpicolinamide

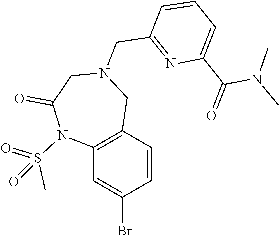 [SMILES: CN(C)C(=O)c1cccc(CN2CC(=O)N(S(C)(=O)=O)c3cc(Br)ccc3C2)n1]

To a mixture of 6-((8-bromo-2-oxo-1,2,3,5-tetrahydro-4H-benzo[e][1,4]diazepin-4-yl)methyl)-N,N-dimethylpicolinamide (Int. 26) (31.0 mg, 0.08 mmol) in THF (2 mL) was added sodium hydride (6.15 mg, 0.15 mmol, 60% in mineral oil) slowly. The reaction was stirred at rt for 10 min then a solution of methanesulfonyl chloride (11.9 μL, 0.15 mmol) was added. The mixture was stirred at rt for 1 h and purified on prepLCMS to give the title compound (20 mg, 0.042 mmol, 54.1% yield). LCMS calc. for C₁₉H₂₂BrN₄O₄S [M+H]⁺: m/z=481.1; Found: 481.2.

### Step 2. 6-((8-(3-((5-acetyl-2-(cyclopropanecarboxamido)pyridin-4-yl)amino)-2-methoxyphenyl)-1-(methylsulfonyl)-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 179 Step 2 using appropriate intermediates, as well as commercial starting materials. LCMS calc. for C₃₇H₄₀N₇O₇S [M+H]⁺: m/z=726.3; Found: 726.4.

### Example 224. 6-((8-(4-((5-acetyl-2-(cyclopropanecarboxamido)pyridin-4-yl)amino)-3-methoxypyridin-2-yl)-6-cyano-1-isopropyl-2-oxo-1,2,3,5-tetrahydro-4H-benzo[e][1,4]diazepin-4-yl)methyl)-N,N-dimethylpicolinamide

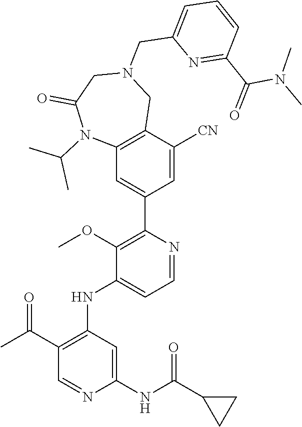 [SMILES: COc1c(Nc2cc(NC(=O)C3CC3)ncc2C(C)=O)ccnc1-c1cc(C#N)c2c(c1)N(C(C)C)C(=O)CN(Cc1cccc(C(=O)N(C)C)n1)C2]

### Step 1. 1-Bromo-2-(bromomethyl)-5-chloro-3-nitrobenzene

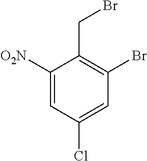 [SMILES: O=[N+]([O-])c1cc(Cl)cc(Br)c1CBr]

A mixture of 1-bromo-5-chloro-2-methyl-3-nitrobenzene (2.62 g, 10.46 mmol) and NBS (2.79 g, 15.69 mmol) in 28 mL of trifluorotoluene was purged with N₂ for 1 minute. Benzoyl peroxide (0.68 g, 2.09 mmol) was added and the mixture was further purged with N₂ for 1 min. The reaction was stirred at 90° C. under N₂ for 6 h. The product mixture was poured into water and was extracted by MTBE twice. The combined organic phase was dried over Na₂SO₄ and concentrated under reduced pressure. The residue obtained was purified by silica gel column chromatography (0-30% DCM/Heptanes) to give the title compound (3.2 g, 9.72 mmol, 92.9% yield). ¹H NMR (300 MHz, CDCl₃) δ 7.89 (d, J=1.2 Hz, 2H), 4.84 (s, 2H).

### Step 2. tert-Butyl (2-bromo-4-chloro-6-nitrobenzyl)glycinate

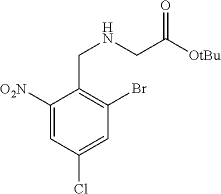 [SMILES: CC(C)(C)OC(=O)CNCc1c(Br)cc(Cl)cc1[N+](=O)[O-]]

To a cold solution of 1-bromo-2-(bromomethyl)-5-chloro-3-nitrobenzene (3.2 g, 9.72 mmol) in DMF (6 mL) was added tert-butyl glycinate (6.37 g, 48.58 mmol). The reaction was stirred at rt for 30 minutes. The reaction was diluted by ethyl acetate and was poured into saturated NH₄Cl solution. The mixture was extracted by ethyl acetate twice. The combined organic phase was dried over Na₂SO₄. After removal of solvent, the residue was purified by silica gel column chromatography (5-40% EtOAc/Heptane) to give the title compound (3.0 g, 7.90 mmol, 81.3% yield) as pale-yellow oil. LC-MS calc. for C₉H₉BrClN₂O₄[M+H—C₄H₈]⁺: m/z=324.9/322.9; Found: 325.1/323.1.

### Step 3. tert-Butyl (2-amino-6-bromo-4-chlorobenzyl)glycinate

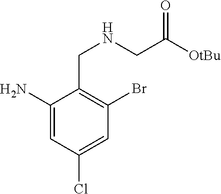 [SMILES: CC(C)(C)OC(=O)CNCc1c(N)cc(Cl)cc1Br]

Zinc (3.10 g, 47.41 mmol) was added portion wise to a mixture of tert-butyl (2-bromo-4-chloro-6-nitrobenzyl)glycinate (3.0 g, 7.9 mmol) and ammonium chloride (5.07 g, 94.83 mmol) in methanol (100 mL) at 0° C. The reaction was brought back to rt and stirred for 1 h. The reaction was diluted by 400 mL of DCM and was filtered through celite. The filtrate was concentrated under reduced pressure. The residue was purified by silica gel column chromatography (0-5% MeOH/DCM) to give the title compound (2.5 g, 7.15 mmol, 90.5% yield) as off-white solids. LC-MS calc. for C₁₃H₁₉BrClN₂O₂[M+H]⁺: m/z=351.0/349.0/353.0; Found: 351.1/349.1/353.1.

### Step 4. tert-Butyl N-(2-amino-6-bromo-4-chlorobenzyl)-N-(tert-butoxycarbonyl)glycinate

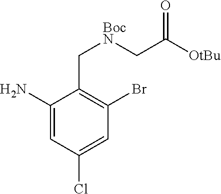 [SMILES: CC(C)(C)OC(=O)CN(Cc1c(N)cc(Cl)cc1Br)C(=O)OC(C)(C)C]

To a cold solution of tert-butyl (2-amino-6-bromo-4-chlorobenzyl)glycinate (2.5 g, 7.15 mmol) in THF (100 mL) was added di-tert butyl dicarbonate (1.72 g, 7.87 mmol). The reaction was stirred at rt for 1 h. The reaction mixture was concentrated under reduced pressure to obtain the title compound (3.2 g, 7.11 mmol, 99.5% yield) as colorless viscous solids which was used without further purification. LC-MS calc. for C₁₈H₂₇BrClN₂O₄[M+H]⁺: m/z=451.1/449.1; Found: 451.3/449.1.

### Step 5. N-(2-Amino-6-bromo-4-chlorobenzyl)-N-(tert-butoxycarbonyl)glycine

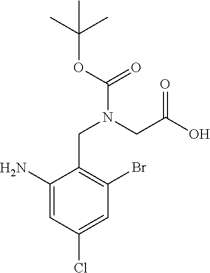 [SMILES: CC(C)(C)OC(=O)N(CC(=O)O)Cc1c(N)cc(Cl)cc1Br]

To a cold solution of tert-butyl N-(2-amino-6-bromo-4-chlorobenzyl)-N-(tert-butoxycarbonyl)glycinate (3.2 g, 7.11 mmol) in THF (10 mL) and methanol (60 mL) was added NaOH (4.27 g, 106.72 mmol) in cold water (60 mL). The reaction was stirred at 55° C. overnight. The product mixture was concentrated to about half its volume and cooled to 0° C. and the pH was adjusted to 3. The acidified mixture was extracted by DCM/MeOH (v/v=15/1) three times. The combined organic phase was dried over Na₂SO₄, filtered and concentrated under reduced pressure to obtain the title compound (2.85 g, 7.24 mmol, 102% yield) as white solids. LC-MS calc. for C₁₀H₁₁BrClN₂O₄[M+H—C₄H₈]⁺: m/z=339.0/337.0; Found: 339.1/337.2.

### Step 6. tert-Butyl 6-bromo-8-chloro-2-oxo-1,2,3,5-tetrahydro-4H-benzo[e][1,4]diazepine-4-carboxylate

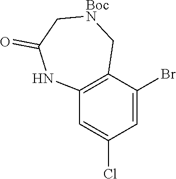 [SMILES: CC(C)(C)OC(=O)N1CC(=O)Nc2cc(Cl)cc(Br)c2C1]

To a solution of N-(2-amino-6-bromo-4-chlorobenzyl)-N-(tert-butoxycarbonyl)glycine (2.85 g, 7.24 mmol) in DCE (150 mL) was added EDC (2.25 g, 14.48 mmol). The reaction mixture was stirred at rt for 5 min, then pyridine (2.33 mL, 28.96 mmol) and DMAP (88.45 mg, 0.72 mmol) was added. The reaction was stirred at rt for 1 h. The reaction was diluted by DCM, washed with sat. NH₄Cl (aq) with a small portion of 1N HCl (aq) and brine. The organic phase was dried over Na₂SO₄. After removal of solvent, the residue was purified by silica gel column chromatography (0-30% EtOAc/DCM) to give the title compound (2.12 g, 5.64 mmol, 78% yield) as white solids. LC-MS calc. for C₁₄H₁₇BrClN₂O₃[M+H]⁺: m/z=377.0/375.0; Found: 377.1/375.2.

### Step 7. tert-Butyl 6-bromo-8-chloro-1-isopropyl-2-oxo-1, 2,3,5-tetrahydro-4H-benzo[e][1,4]diazepine-4-carboxylate

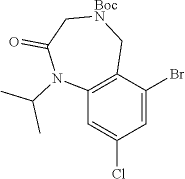 [SMILES: CC(C)N1C(=O)CN(C(=O)OC(C)(C)C)Cc2c(Br)cc(Cl)cc21]

To a mixture of tert-butyl 6-bromo-8-chloro-2-oxo-1,2,3,5-tetrahydro-4H-benzo[e]-[1,4]diazepine-4-carboxylate (0.84 g, 2.24 mmol) and Cs₂CO₃ (2.19 g, 6.71 mmol) in DMSO (10 mL) was added 2-iodopropane (0.56 mL, 5.59 mmol). The reaction was stirred at 40° C. for 90 mins under N₂. The reaction was diluted by ethyl acetate and was poured into cold water. The mixture was extracted by MTBE twice. The combined organic layer was washed by brine and dried over Na₂SO₄, filtered, and concentrated under reduced pressure. The residue obtained was purified by silica gel column chromatography (DCM then 10% MTBE in DCM) To yield the title compound (817 mg, 1.96 mmol, 87.5% yield) as colorless viscous solids. LC-MS calc. for C₁₇H₂₃BrClN₂O₃[M+H]⁺: m/z=419.1/417.1; Found: 419.2/417.2.

### Step 8. tert-Butyl 8-chloro-6-cyano-1-isopropyl-2-oxo-1,2,3,5-tetrahydro-4H-benzo[e][1,4]diazepine-4-carboxylate

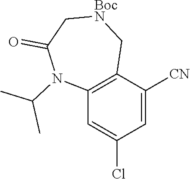 [SMILES: CC(C)N1C(=O)CN(C(=O)OC(C)(C)C)Cc2c(C#N)cc(Cl)cc21]

A mixture of tert-butyl 6-bromo-8-chloro-1-isopropyl-2-oxo-1,2,3,5-tetrahydro-4H-benzo[e][1,4]diazepine-4-carboxylate (350 mg, 0.84 mmol), zinc cyanide (59 mg, 0.5 mmol), zinc (11 mg, 0.17 mmol), and Pd(PPh₃)₄ (145 mg, 0.13 mmol) was dissolved in DMF (6 mL) and water (6 μL). The reaction mixture was purged with N₂ for 2 min, sealed, and stirred at 120° C. for 1 h. The reaction was cooled to rt and was diluted by ethyl acetate. The mixture was poured into sat. NaHCO₃ (aq) solution and was extracted by ethyl acetate twice. The combined organic phase was diluted by heptanes and was washed by saturated NH₄Cl solution. The organic phase was dried over Na₂SO₄, filtered and concentrated under reduced pressure. The residue was purified by silica gel column chromatography (0-30% MTBE/Heptanes) to give the title compound (175 mg, 0.48 mmol, 57.4% yield) as white solids. LC-MS calc. for C₁₈H₂₂ClN₃NaO₃ [M+Na]⁺: m/z=386.1; Found: 386.3.

### Step 9. 6-((8-Chloro-6-cyano-1-isopropyl-2-oxo-1,2,3,5-tetrahydro-4H-benzo[e]-[1,4]diazepin-4-yl)methyl)-N,N-dimethylpicolinamide

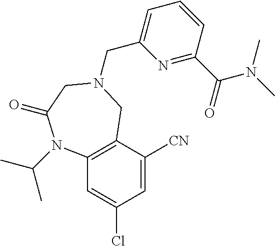 [SMILES: CC(C)N1C(=O)CN(Cc2cccc(C(=O)N(C)C)n2)Cc2c(C#N)cc(Cl)cc21]

The title compound was prepared in accordance with the synthetic protocols set forth in Example 176 Step 6 using appropriate intermediates, as well as commercial starting materials. LC-MS calc. for C₂₂H₂₅ClN₅O₂[M+H]⁺: m/z=426.2; Found: 426.2.

### Step 10. (6-Cyano-4-((6-(dimethylcarbamoyl)pyridin-2-yl)methyl)-1-isopropyl-2-oxo-2,3,4,5-tetrahydro-1H-benzo[e][1,4]diazepin-8-yl)boronic acid

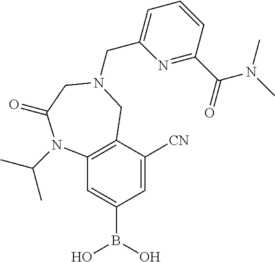 [SMILES: CC(C)N1C(=O)CN(Cc2cccc(C(=O)N(C)C)n2)Cc2c(C#N)cc(B(O)O)cc21]

A mixture of 6-((8-chloro-6-cyano-1-isopropyl-2-oxo-1,2,3,5-tetrahydro-4H-benzo[e]-[1,4]diazepin-4-yl)methyl)-N,N-dimethylpicolinamide (125.0 mg, 0.29 mmol) and potassium acetate (86.42 mg, 0.88 mmol) was dissolved in ethanol (3 mL). The mixture was purged with N₂ for 1 minute then Xphos Pd G2 (23.09 mg, 0.03 mmol), XPhos (13.99 mg, 0.03 mmol) and B₂(OH)₄ (92.09 mg, 1.03 mmol) were added. The reaction mixture was further purged with N₂ for 1 minute and stirred at 50° C. for 45 min. The reaction was cooled to rt, diluted by cold water and was extracted by DCM/MeOH (v/v=15/1) three times. The combined organic phase was dried over Na₂SO₄, filtered and concentrated under reduced pressure. The residue obtained was purified by prep-HPLC on C₁₈ column (30×250 mm, 10 m) using mobile phase 10% to 50% MeCN/H₂O (w/0.1% formic acid). The desired fractions were collected, concentrated to dryness to give the title compound (92 mg, 0.21 mmol, 72.0% yield) as white solids. LC-MS calc. for C₂₂H₂₇BN₅O₄ [M+H]⁺: m/z=436.2; Found: 436.3.

### Step 11. 6-((8-(4-((5-Acetyl-2-(cyclopropanecarboxamido)pyridin-4-yl)amino)-3-methoxypyridin-2-yl)-6-cyano-1-isopropyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 176 Step 8 using appropriate intermediates, as well as commercial starting materials. LC-MS calc. for C₃₉H₄₂N₉O₅ [M+H]⁺: m/z=716.3; Found: 716.5. ¹H NMR (300 MHz, Methanol-d₄) δ 8.90 (s, 1H), 8.44 (s, 1H), 8.40 (d, J=5.1 Hz, 1H), 8.27 (s, 1H), 8.20 (s, 1H), 7.97 (t, J=7.7 Hz, 1H), 7.71 (t, J=7.1 Hz, 2H), 7.50 (d, J=7.4 Hz, 1H), 4.80-4.69 (m, 1H), 4.06 (bs, 4H), 3.63 (s, 3H), 3.18 (s, 2H), 3.13 (s, 3H), 3.04 (s, 3H), 2.68 (s, 3H), 1.93 (s, 1H), 1.30 (d, J=13.9 Hz, 6H), 1.07-0.88 (m, 4H).

### Example 225. (S)-6-((8-(4-((5-acetyl-2-(cyclopropanecarboxamido)pyridin-4-yl)amino)-3-methoxypyridin-2-yl)-1-(1,1-difluoropropan-2-yl)-2-oxo-1,2,3,5-tetrahydro-4H-pyrido[3,2-e][1,4]diazepin-4-yl)methyl)-N,N-dimethylpicolinamide

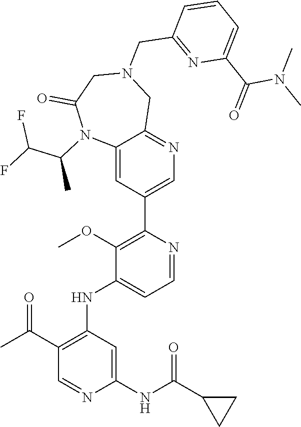 [SMILES: COc1c(Nc2cc(NC(=O)C3CC3)ncc2C(C)=O)ccnc1-c1cnc2c(c1)N([C@@H](C)C(F)F)C(=O)CN(Cc1cccc(C(=O)N(C)C)n1)C2]

### Step 1. Ethyl ((3-amino-5-bromopyridin-2-yl)methyl)glycinate

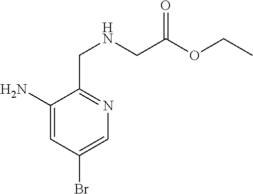 [SMILES: CCOC(=O)CNCc1ncc(Br)cc1N]

The title compound was prepared in accordance with the synthetic protocols set forth in Int. 28 Step 1 using the appropriate intermediates and commercial starting materials. LC-MS calc. for C₁₀H₁₅BrN₃O₂[M+H]⁺: m/z=288.0/290.0; Found: 288.0/290.0.

### Step 2. Ethyl N-((3-amino-5-bromopyridin-2-yl)methyl)-N-(tert-butoxycarbonyl)glycinate

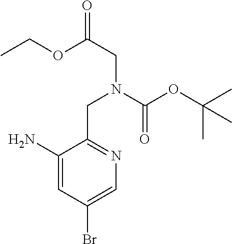 [SMILES: CCOC(=O)CN(Cc1ncc(Br)cc1N)C(=O)OC(C)(C)C]

The title compound was prepared in accordance with the synthetic protocols set forth in Example 224 Step 4 using the appropriate intermediates. LC-MS calc. for C₁₅H₂₃BrN₃O₄[M+H]⁺: m/z=388.1/390.1; Found: 388.1/390.1.

### Step 3. Ethyl N-((5-bromo-3-iodopyridin-2-yl)methyl)-N-(tert-butoxycarbonyl)glycinate

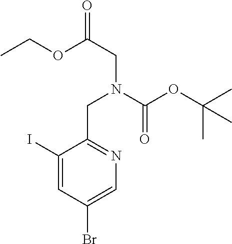 [SMILES: CCOC(=O)CN(Cc1ncc(Br)cc1I)C(=O)OC(C)(C)C]

tert-Butyl nitrite (1.88 mL, 15.9 mmol) was added dropwise to a solution of ethyl N-((3-amino-5-bromopyridin-2-yl)methyl)-N-(tert-butoxycarbonyl)glycinate (4.4 g, 11.3 mmol) and copper (I) iodide (3.24 g, 17 mmol) in MeCN (38 mL) at 0° C. The reaction mixture was then stirred at 60° C. for 4 hours. The reaction mixture was directly condensed, then taken up in 3:1 CHCl₃/IPA and centrifuged. The solids were washed with 3:1 CHCl₃/IPA (×2) and centrifuged again. The combined organic phases were washed with sat. NaHCO₃ (aq.), dried, condensed, and purified by SiO₂ FCC (0-15% EtOAc/heptane) to afford the title compound (3 g, 53% yield) as a white solid. LC-MS calc. for C₁₅H₂₁BrIN₂O₄ [M+H]⁺: m/z=499.0/501.0; Found: 498.9/500.9.

### Step 4. (S)-6-((8-(4-((5-Acetyl-2-(cyclopropanecarboxamido)pyridin-4-yl)amino)-3-methoxypyridin-2-yl)-1-(1,1-difluoropropan-2-yl)-2-oxo-1,2,3,5-tetrahydro-4H-pyrido[3,2-e][1,4]diazepin-4-yl)methyl)-N,N-dimethylpicolinamide

The title compound was prepared as the formate salt in accordance with the synthetic protocols set forth in Example 176 Steps 3 through 8 using the appropriate intermediates and commercial starting materials. LC-MS calc. for C₃₇H₄₀F₂N₉O₅[M+H]⁺: m/z=728.3; Found: 728.3.

### Example 226. (R)-6-((8-(4-((5-Acetyl-2-(cyclopropanecarboxamido)pyridin-4-yl)amino)-3-methoxypyridin-2-yl)-1-(1,1-difluoropropan-2-yl)-2-oxo-1,2,3,5-tetrahydro-4H-pyrido[3,2-e][1,4]diazepin-4-yl)methyl)-N,N-dimethylpicolinamide

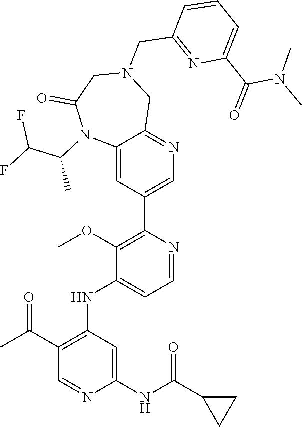 [SMILES: COc1c(Nc2cc(NC(=O)C3CC3)ncc2C(C)=O)ccnc1-c1cnc2c(c1)N([C@H](C)C(F)F)C(=O)CN(Cc1cccc(C(=O)N(C)C)n1)C2]

The title compound was prepared as a formate salt in accordance with the synthetic protocols set forth in Example 225 using the appropriate intermediates and commercial materials. LC-MS calc. for C₃₇H₄₀F₂N₉O₅[M+H]⁺: m/z=728.3; Found: 728.4.

### Example 227. 6-((8-(4-((5-Acetyl-2-(cyclopropanecarboxamido)pyridin-4-yl)amino)-3-methoxypyridin-2-yl)-6-(ethylamino)-1-isopropyl-2-oxo-1,2,3,5-tetrahydro-4H-benzo[e][1,4]diazepin-4-yl)methyl)-N,N-dimethylpicolinamide

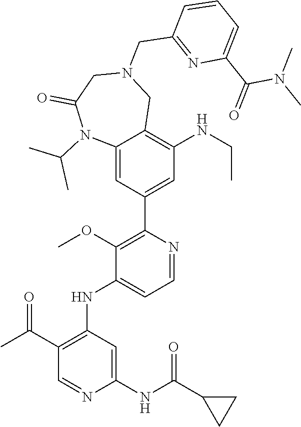 [SMILES: CCNc1cc(-c2nccc(Nc3cc(NC(=O)C4CC4)ncc3C(C)=O)c2OC)cc2c1CN(Cc1cccc(C(=O)N(C)C)n1)CC(=O)N2C(C)C]

### Step 1. tert-Butyl 8-chloro-6-(ethylamino)-1-isopropyl-2-oxo-1,2,3,5-tetrahydro-4H-benzo[e][1,4]diazepine-4-carboxylate

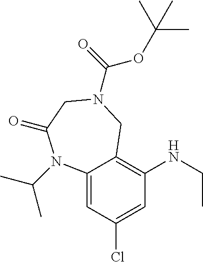 [SMILES: CCNc1cc(Cl)cc2c1CN(C(=O)OC(C)(C)C)CC(=O)N2C(C)C]

A mixture of tert-butyl 6-bromo-8-chloro-1-isopropyl-2-oxo-1,2,3,5-tetrahydro-4H-benzo[e][1,4]diazepine-4-carboxylate (314.0 mg, 0.75 mmol), Cs₂CO₃ (734.7 mg, 2.26 mmol), Pd(OAc)₂ (16.9 mg, 0.08 mmol), and BINAP (117.01 mg, 0.19 mmol) was dissolved in toluene (8 mL). The reaction mixture was purged with N₂ for 1 min then ethylamine 2.0M in THF (2.26 mL, 4.51 mmol) was added. The reaction mixture was purged with N₂ for 30 seconds, sealed and was stirred at 90° C. for 2 hours. The reaction was poured into the brine and extracted by ethyl acetate twice. The combined organic phase was dried over Na₂SO₄, filtered and concentrated under reduced pressure. The residue obtained was purified by silica gel column chromatography (0-35% EtOAc/Heptane) to give the title compound (224 mg, 0.59 mmol, 78.0% yield) as colorless viscous solids. LC-MS calc. for C₁₉H₂₉ClN₃O₃[M+H]⁺: m/z=382.2; Found: 382.4.

### Step 2. 6-((8-Chloro-6-(ethylamino)-1-isopropyl-2-oxo-1,2,3,5-tetrahydro-4H-benzo[e][1,4]diazepin-4-yl)methyl)-N,N-dimethylpicolinamide

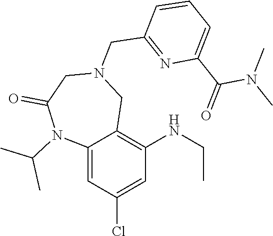 [SMILES: CCNc1cc(Cl)cc2c1CN(Cc1cccc(C(=O)N(C)C)n1)CC(=O)N2C(C)C]

To a cold solution of tert-butyl 8-chloro-6-(ethylamino)-1-isopropyl-2-oxo-1,2,3,5-tetrahydro-4H-benzo[e][1,4]diazepine-4-carboxylate (175.0 mg, 0.46 mmol) in DCM (4 mL) was added trifluoroacetic acid (2 mL). The reaction was stirred at rt for 1 h. The reaction was concentrated under reduced pressure. The residue was dissolved in toluene and concentrated under reduced pressure. The residue and 6-formyl-N,N-dimethylpicolinamide (Int. 24) (98.0 mg, 0.55 mmol) was dissolved in DCM (4 mL). The mixture was stirred at rt for 15 mins then sodium triacetoxyborohydride (291.4 mg, 1.37 mmol) was added. The reaction mixture was stirred at rt for 3 hours. The product mixture was poured into sat. NaHCO₃ (aq) solution and was extracted by DCM/MeOH (v/v=15/1) three times. The combined organic phase was dried over Na₂SO₄, filtered, and concentrated under reduced pressure. The residue obtained was purified by silica gel column chromatography (0-8% MeOH/DCM) to give the title compound (150 mg, 0.34 mmol, 73.7% yield) as colorless viscous solids. LC-MS calc. for C₂₃H₃₁ClN₅O₂[M+H]⁺: m/z=444.2; Found: 444.3.

### Step 3. (4-((6-(Dimethylcarbamoyl)pyridin-2-yl)methyl)-6-(ethylamino)-1-isopropyl-2-oxo-2,3,4,5-tetrahydro-1H-benzo[e][1,4]diazepin-8-yl)boronic acid

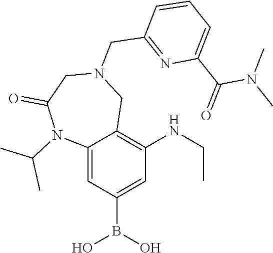 [SMILES: CCNc1cc(B(O)O)cc2c1CN(Cc1cccc(C(=O)N(C)C)n1)CC(=O)N2C(C)C]

A mixture of 6-((8-chloro-6-(ethylamino)-1-isopropyl-2-oxo-1,2,3,5-tetrahydro-4H-benzo[e][1,4]diazepin-4-yl)methyl)-N,N-dimethylpicolinamide (95.0 mg, 0.21 mmol), potassium acetate (63.0 mg, 0.64 mmol), tetrahydroxydiboron (76.73 mg, 0.86 mmol), Xphos Pd G2 (16.84 mg, 0.02 mmol) and Xphos (10.2 mg, 0.02 mmol) was dissolved in EtOH (3 mL) and ethylene glycol (300 μL). The reaction mixture was purged with N₂ for 1 minute, sealed, and stirred at 60° C. for 45 min. The product mixture was diluted by cold water and a small portion of sat. NaHCO₃ (aq) solution. The diluted mixture was extracted by DCM/MeOH (v/v=10/1) three times. The combined organic phase was dried over Na₂SO₄, filtered and concentrated under reduced pressure. The residue obtained was purified by silica gel column chromatography (0-10% MeOH/DCM) to give the title compound (85 mg, 0.19 mmol, 87.6% yield) as colorless viscous solids. LC-MS calc. for C₂₃H₃₃BN₅O₄[M+H]⁺: m/z=454.3; Found: 454.4.

### Step 4. 6-((8-(4-((5-Acetyl-2-(cyclopropanecarboxamido)pyridin-4-yl)amino)-3-methoxypyridin-2-yl)-6-(ethylamino)-1-isopropyl-2-oxo-1,2,3,5-tetrahydro-4H-benzo[e][1,4]-diazepin-4-yl)methyl)-N,N-dimethylpicolinamide

The title compound was prepared in accordance with the synthetic protocols set forth in Example 176 Step 8 using appropriate intermediates, as well as commercial starting materials. LC-MS calc. for C₄₀H₄₈N₉O₅ [M+H]⁺: m/z=734.4; Found: 734.6. ¹H NMR (300 MHz, Methanol-d₄) δ 9.04 (s, 1H), 8.49 (s, 1H), 8.47 (d, J=6.7 Hz, 1H), 8.09 (t, J=7.8 Hz, 1H), 8.01 (d, J=6.7 Hz, 1H), 7.68 (t, J=7.6 Hz, 2H), 7.17 (s, 2H), 4.96 (d, J=14.1 Hz, 1H), 4.74 (p, J=6.8 Hz, 1H), 4.57 (d, J=3.4 Hz, 2H), 4.18 (d, J=14.1 Hz, 1H), 3.86-3.66 (m, 5H), 3.38-3.32 (m, 2H), 3.16 (s, 3H), 3.06 (s, 3H), 2.73 (s, 3H), 2.03-1.87 (m, 1H), 1.56 (d, J=6.7 Hz, 3H), 1.42-1.28 (m, 6H), 1.12-0.93 (m, 4H).

### Example 228. 6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-pyrido[2,3-e][1,4]diazepin-4-yl)methyl)-N,N-dimethylpicolinamide

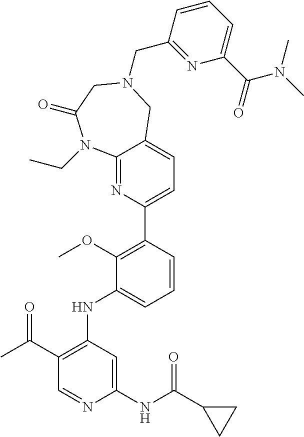 [SMILES: CCN1C(=O)CN(Cc2cccc(C(=O)N(C)C)n2)Cc2ccc(-c3cccc(Nc4cc(NC(=O)C5CC5)ncc4C(C)=O)c3OC)nc21]

### Step 1. 3-(Aminomethyl)-6-chloropyridin-2-amine

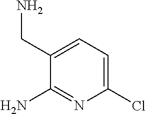

To a solution of 2-amino-6-chloronicotinonitrile (1.0 g, 6.51 mmol) in THF (20 mL) was added borane dimethyl sulfide (0.90 g, 65.117 mmol). The reaction was stirred at 45° C. for 2 hours. The reaction mixture was cooled to 0° C. and quenched by dropwise addition of methanol. The reaction mixture was concentrated under reduced pressure. The residue obtained was dissolved in 4 N HCl in IPA and stirred at rt overnight. The reaction mixture was concentrated under reduced pressure to the title compound (1.5 g, 6.51 mmol, 99.9% yield) as the HCl salt, which was used in the next step without further purification. LC-MS calc. for C₆H₆ClN₂ [M−H₂N]⁺: m/z=141.1; Found: 141.3.

### Step 2. Ethyl ((2-amino-6-chloropyridin-3-yl)methyl)glycinate

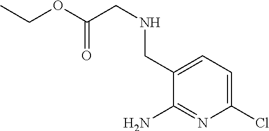 [SMILES: CCOC(=O)CNCc1ccc(Cl)nc1N]

To a mixture of 3-(aminomethyl)-6-chloropyridin-2-amine; dihydrochloride (1.5 g, 6.51 mmol) in THF (20 mL) was added triethylamine (3.29 g, 32.54 mmol) and ethyl bromoacetate (1.63 g, 9.76 mmol). The reaction mixture was stirred at rt for 30 minutes. Then the reaction mixture was heated to 50° C. and stirred for 1 hour. The reaction was concentrated, diluted by DCM, poured into sat. NaHCO₃ (aq) solution and was extracted by DCM/MeOH (v/v=10/1) three times. The combined organic phase was dried over Na₂SO₄, filtered and concentrated under reduced pressure. The residue obtained was purified by silica gel column chromatography (0-6% MeOH/DCM) to give the title compound (380 mg, 1.56 mmol, 24.0% yield) as light-yellow viscous solids. LC-MS calc. for C₁₀H₁₅ClN₃O₂[M+H]⁺: m/z=244.1; Found: 244.4.

### Step 3. Ethyl N-((2-amino-6-chloropyridin-3-yl)methyl)-N-((6-(dimethylcarbamoyl)-pyridin-2-yl)methyl)glycinate

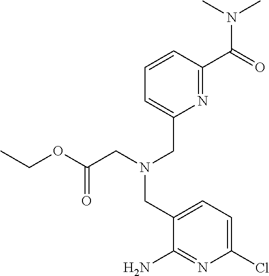 [SMILES: CCOC(=O)CN(Cc1cccc(C(=O)N(C)C)n1)Cc1ccc(Cl)nc1N]

To a mixture of ethyl 2-[(2-amino-6-chloro-3-pyridinyl)methylamino]acetate (330.0 mg, 1.35 mmol) and 6-(bromomethyl)-N,N-dimethylpicolinamide (Int. 23) (362.1 mg, 1.49 mmol) in DMF (4 mL) was added Cs₂CO₃ (573.6 mg, 1.76 mmol). The reaction mixture wad stirred at rt for 5 hours. The reaction was diluted by MTBE and poured into cold water. The reaction mixture was extracted by MTBE 3 times. The combined organic phase was washed by brine and dried over Na₂SO₄, filtered, and concentrated under reduced pressure. The residue obtained was purified by silica gel column chromatography (0-6% MeOH/DCM) to give the title compound (550 mg, 1.08 mmol, 80.1% yield) with 80% purity. LC-MS calc. for C₁₉H₂₅ClN₅O₃[M+H]⁺: 406.2; Found: 406.4.

### Step 4. N-((2-Amino-6-chloropyridin-3-yl)methyl)-N-((6-(dimethylcarbamoyl)pyridin-2-yl)methyl)glycine; trihydrochloride

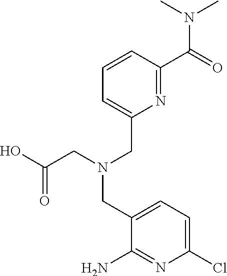 [SMILES: CN(C)C(=O)c1cccc(CN(CC(=O)O)Cc2ccc(Cl)nc2N)n1]

To a cold solution of ethyl N-((2-amino-6-chloropyridin-3-yl)methyl)-N-((6-(dimethyl-carbamoyl)pyridin-2-yl)methyl)glycinate (550.0 mg, 1.08 mmol) in cold methanol (4 mL) was added NaOH (130.0 mg, 3.25 mmol) in water (4 mL). The reaction was stirred at rt for 1 h. The reaction was neutralized by TFA at 0° C. The resulting solution was directly purified by prep-HPLC on C₁₈ column (30×250 mm, 10 m) using mobile phase 6% to 30% MeCN/H₂O (w/0.05% TFA) (tR=18 min). The desired fractions were collected, concentrated under reduced pressure. The residue obtained was dissolved in THF and 4 N HCl in dioxane. A precipitate formed. The resulting mixture was sonicated briefly and concentrated under reduced pressure to give the HCl salt of the title compound (380 mg, 72% yield) as white solids. LC-MS calc. for C₁₇H₂₁ClN₅O₃[M+H]⁺: m/z=378.1; Found: 378.4.

### Step 5. 6-((8-Chloro-2-oxo-1,2,3,5-tetrahydro-4H-pyrido[2,3-e][1,4]diazepin-4-yl)methyl)-N,N-dimethylpicolinamide

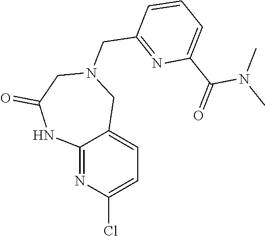 [SMILES: CN(C)C(=O)c1cccc(CN2CC(=O)Nc3nc(Cl)ccc3C2)n1]

To a cold solution of N-((2-amino-6-chloropyridin-3-yl)methyl)-N-((6-(dimethyl-carbamoyl)pyridin-2-yl)methyl)glycine; trihydrochloride (380.0 mg, 0.78 mmol) in DMF (6 mL) was added pyridine (0.63 mL, 7.8 mmol) followed by propylphosphonic anhydride (2.32 mL, 3.9 mmol, 50 wt % in THF solution). The reaction was stirred at rt for 1 h. Another portion of pyridine (0.63 mL, 7.8 mmol) and propylphosphonic anhydride (2.48 mL, 3.90 mmol, 50 wt % in THF solution) was added to the reaction. The reaction was stirred for another hour. The reaction was poured into cold water and was extracted by DCM/MeOH (v/v=15/1) three times. The combined organic phase was dried over Na₂SO₄, filtered, and concentrated under reduced pressure. The residue obtained was purified by prep-HPLC on C₁₈ column (30×250 mm, 10 m) using mobile phase 6% to 30% MeCN/H₂O (w/0.05% TFA) (tR=18 min). The desired fractions were collected, concentrated and poured into sat. NaHCO₃ (aq) solution and were extracted by DCM/MeOH (v/v=15/1) three times. The combined organic phase was dried over Na₂SO₄, filtered and concentrated under reduced pressure to obtain the title compound (210 mg, 74.8% yield) as colorless viscous solids. LC-MS calc. for C₁₇H₁₉ClN₅O₂[M+H]⁺: m/z=360.1; Found: 360.3.

### Step 6. 6-((8-Chloro-1-ethyl-2-oxo-1,2,3,5-tetrahydro-4H-pyrido[2,3-e][1,4]diazepin-4-yl)methyl)-N,N-dimethylpicolinamide

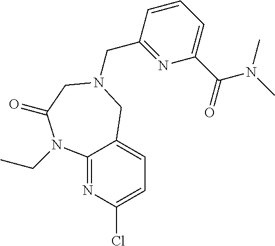 [SMILES: CCN1C(=O)CN(Cc2cccc(C(=O)N(C)C)n2)Cc2ccc(Cl)nc21]

To a mixture of 6-((8-chloro-2-oxo-1,2,3,5-tetrahydro-4H-pyrido[2,3-e][1,4]diazepin-4-yl)methyl)-N,N-dimethylpicolinamide (195.0 mg, 0.54 mmol) and Cs₂CO₃ (441.4 mg, 1.35 mmol) in DMF (3 mL) was added iodoethane (101.4 mg, 0.65 mmol). The reaction mixture was stirred at rt for 1 h. The reaction was filtered. Three drops of TFA were added to the filtrate, and the filtrate was directly purified by prep-HPLC on C₁₈ column (30×250 mm, 10 m) using mobile phase 8% to 55% MeCN/H₂O (w/0.1% TFA) (tR=18 min). The desired fractions were collected, concentrated, poured into sat. NaHCO₃ (aq) solution and were extracted by DCM/MeOH (v/v=15/1) three times. The combined organic phase was dried over Na₂SO₄, filtered and concentrated under reduced pressure to obtain the title compound (160 mg, 0.41 mmol, 76.1% yield) as colorless oil. LC-MS calc. for C₁₉H₂₃ClN₅O₂[M+H]⁺: m/z=388.2; Found: 388.3.

### Step 7. 6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-pyrido[2,3-e][1,4]diazepin-4-yl)methyl)-N,N-dimethylpicolinamide

A mixture of 6-((8-chloro-1-ethyl-2-oxo-1,2,3,5-tetrahydro-4H-pyrido[2,3-e][1,4]-diazepin-4-yl)methyl)-N,N-dimethylpicolinamide (18.9 mg, 0.05 mmol), [3-[[5-acetyl-2-(cyclopropanecarbonylamino)-4-pyridinyl]amino]-2-methoxyphenyl]boronic acid (18.0 mg, 0.05 mmol), cesium acetate (28.08 mg, 0.15 mmol), and Pd(dppf)Cl₂ (5.35 mg, 0.01 mmol) was dissolved in 1,4-dioxane (1.5 mL) and water (0.08 mL). The reaction mixture was purged with N₂ for 1 minutes, sealed, and stirred at 98° C. overnight. The product mixture was diluted by MeCN and filtered through a syringe filter. The filtrate was concentrated under reduced pressure. The residue obtained was purified by prep-HPLC on C₁₈ column (30×250 mm, 10 m) using mobile phase 8% to 55% MeCN/H₂O (w/0.1% TFA) (tR=18 min). The desired fractions were collected, concentrated and freeze-dried to give the title compound (17.2 mg, 0.025 mmol, 52.1% yield) as white solids. LC-MS calc. for C₃₇H₄₁N₈O₅ [M+H]⁺: m/z=677.3; Found: 677.6. ¹H NMR (300 MHz, Methanol-d₄) δ 8.77 (s, 1H), 8.05 (t, J=7.0 Hz, 1H), 8.01 (d, J=6.4 Hz, 1H), 7.94 (d, J=7.9 Hz, 1H), 7.90 (dd, J=7.8, 1.7 Hz, 1H), 7.68 (d, J=7.1 Hz, 1H), 7.65-7.56 (m, 2H), 7.44 (t, J=7.9 Hz, 1H), 6.74 (d, J=4.4 Hz, 1H), 4.51 (s, 2H), 4.24 (d, J=7.3 Hz, 4H), 3.71 (s, 2H), 3.62 (s, 3H), 3.15 (s, 3H), 3.05 (s, 3H), 2.72 (s, 3H), 1.78 (tt, J=7.7, 4.3 Hz, 1H), 1.34 (t, J=7.1 Hz, 3H), 1.17-0.98 (m, 4H).

### Example 229. 6-((8-(3-((5-Acetyl-2-(cyclopropanecarboxamido)pyridin-4-yl)amino)-2-methoxyphenyl)-6-ethoxy-1-ethyl-2-oxo-1,2,3,5-tetrahydro-4H-benzo[e][1,4]diazepin-4-yl)methyl)-N,N-dimethylpicolinamide

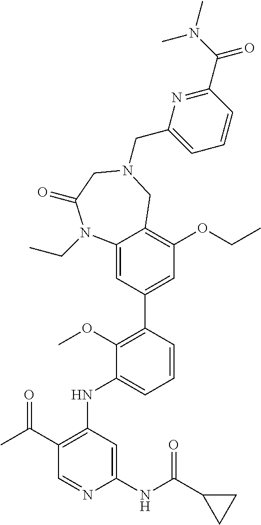 [SMILES: CCOc1cc(-c2cccc(Nc3cc(NC(=O)C4CC4)ncc3C(C)=O)c2OC)cc2c1CN(Cc1cccc(C(=O)N(C)C)n1)CC(=O)N2CC]

### Step 1. 4-Bromo-2-hydroxy-6-nitrobenzaldehyde

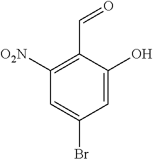 [SMILES: O=Cc1c(O)cc(Br)cc1[N+](=O)[O-]]

3-bromo-5-nitrophenol (3.0 g, 13.76 mmol) and hexamethylenetetramine (4.05 g, 28.9 mmol) was dissolved in trifluoroacetic acid (33 mL). The reaction was sealed and stirred at 100° C. for 20 hours. The reaction mixture was concentrated under reduced pressure. The residue obtained was diluted with ethyl acetate and water. The pH of aqueous layer was adjusted to pH~3 by sat. NaHCO₃ (aq) solution and extracted with ethyl acetate 3 times. The combined organic phase was dried over Na₂SO₄, filtered and concentrated under reduced pressure. The residue obtained was purified by silica gel column chromatography (5-40% EtOAc/Heptanes) to give a mixture of 4-bromo-2-hydroxy-6-nitrobenzaldehyde and 2-bromo-6-hydroxy-4-nitrobenzaldehyde (total 1.75 g, 7.11 mmol, 51.7% yield) as a yellow oil. LC-MS [M−H]⁻ calc. for C₇H₃BrNO₄: 243.9/245.9; Found: 244.3/246.3.

### Step 2. 4-Bromo-2-ethoxy-6-nitrobenzaldehyde

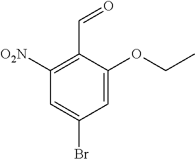

To a mixture of 4-bromo-2-hydroxy-6-nitrobenzaldehyde and 2-bromo-6-hydroxy-4-nitrobenzaldehyde (total 1.65 g, 6.71 mmol) in DMSO (16 mL) was added iodoethane (1.62 mL, 20.1 mmol) and K₂CO₃ (1.85 g, 13.4 mmol). The reaction was stirred at rt for 6 h. The reaction was diluted by MTBE, poured into water, and was extracted by MTBE 3 times. The combined organic phase was washed by brine, dried over Na₂SO₄, filtered, and concentrated under reduced pressure. The residue obtained was purified by silica gel column chromatography (0-20% MTBE/Heptanes) to give a mixture of 4-bromo-2-ethoxy-6-nitrobenzaldehyde and 2-bromo-6-ethoxy-4-nitrobenzaldehyde (total 1.72 g, 6.28 mmol, 93.6% yield). (Rf=0.5 and 0.52, MTBE/Heptanes=1/3).

### Step 3. 6-[(8-Bromo-6-ethoxy-2-oxo-3,5-dihydro-1H-1,4-benzodiazepin-4-yl)methyl]-N,N-dimethylpyridine-2-carboxamide

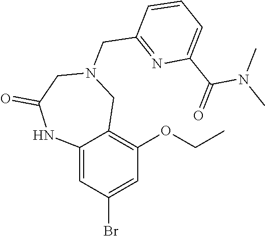 [SMILES: CCOc1cc(Br)cc2c1CN(Cc1cccc(C(=O)N(C)C)n1)CC(=O)N2]

The title compound was prepared in accordance with the synthetic protocols set forth in Int. 25 Steps 1 through 2 and Int. 26 Step 1 using appropriate intermediates, as well as commercial starting materials. LC-MS calc. for C₂₀H₂₄BrN₄O₃[M+H]⁺:447.1/449.1; Found: 447.3/449.3.

### Step 4. 6-((8-(3-((5-Acetyl-2-(cyclopropanecarboxamido)pyridin-4-yl)amino)-2-methoxyphenyl)-6-ethoxy-1-ethyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 36 Steps 1 through 2 using appropriate intermediates, as well as commercial starting materials. LC-MS calc. for C₄₀H₄₆N₇O₆ [M+H]⁺: m/z=720.4; Found: 720.6. ¹H NMR (300 MHz, Methanol-d₄) δ 8.78 (s, 1H), 8.07 (t, J=7.8 Hz, 1H), 7.66 (d, J=2.2 Hz, 1H), 7.63 (d, J=2.2 Hz, 1H), 7.57 (dd, J=7.8, 1.5 Hz, 1H), 7.51 (dd, J=7.7, 1.5 Hz, 1H), 7.45-7.35 (m, 2H), 7.29 (s, 1H), 7.01 (d, J=4.7 Hz, 1H), 4.60 (bs, 4H), 4.22 (q, J=6.8 Hz, 2H), 4.09 (bs, 2H), 3.81 (s, 2H), 3.51 (s, 3H), 3.16 (s, 3H), 3.07 (s, 3H), 2.71 (s, 3H), 1.82 (tt, J=7.8, 4.7 Hz, 1H), 1.35 (t, J=6.9 Hz, 3H), 1.26 (t, J=7.1 Hz, 3H), 1.13-0.97 (m, 4H).

### Example 230. 6-((8-(4-((5-Acetyl-2-(cyclopropanecarboxamido)pyridin-4-yl)amino)-3-methoxypyridin-2-yl)-6-ethoxy-1-ethyl-2-oxo-1,2,3,5-tetrahydro-4H-benzo[e][1,4]diazepin-4-yl)methyl)-N,N-dimethylpicolinamide

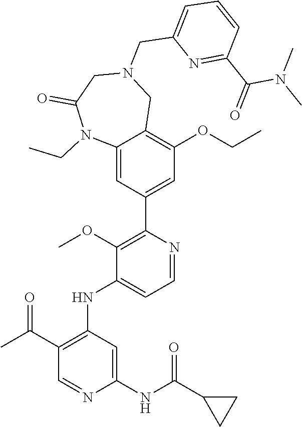 [SMILES: CCOc1cc(-c2nccc(Nc3cc(NC(=O)C4CC4)ncc3C(C)=O)c2OC)cc2c1CN(Cc1cccc(C(=O)N(C)C)n1)CC(=O)N2CC]

The title compound was prepared in accordance with the synthetic protocols set forth in Example 176 Steps 7 through 8 using appropriate intermediates, as well as commercial starting materials. LCMS calc. for C₃₉H₄₅N₈O₆ [M+H]⁺: m/z=721.35; Found: 721.69. ¹H NMR (600 MHz, DMSO-d₆) δ 11.52 (s, 1H), 11.17 (s, 1H), 8.95 (s, 1H), 8.42 (d, J=5.3 Hz, 1H), 8.38 (s, 1H), 7.92 (t, J=7.7 Hz, 1H), 7.57 (dd, J=20.3, 6.4 Hz, 3H), 7.47 (s, 1H), 7.43 (d, J=7.6 Hz, 1H), 4.11 (q, J=6.9 Hz, 2H), 3.92 (d, J=5.9 Hz, 2H), 3.82 (s, 2H), 3.75 (d, J=9.9 Hz, 2H), 3.56 (s, 3H), 3.05 (s, 2H), 3.00 (s, 3H), 2.92 (s, 3H), 2.68 (s, 3H), 2.10-2.03 (m, 1H), 1.30 (t, J=6.9 Hz, 3H), 1.11 (t, J=7.1 Hz, 3H), 0.86 (d, J=7.7 Hz, 4H).

### Example 231. 6-((8-(4-((5-Acetyl-2-(cyclopropanecarboxamido)pyridin-4-yl)amino)-3-methoxypyridin-2-yl)-1-isopropyl-6-methoxy-2-oxo-1,2,3,5-tetrahydro-4H-benzo[e][1,4]diazepin-4-yl)methyl)-N,N-dimethylpicolinamide

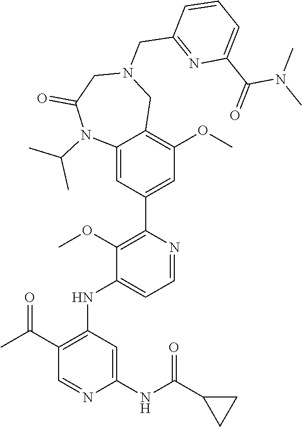 [SMILES: COc1cc(-c2nccc(Nc3cc(NC(=O)C4CC4)ncc3C(C)=O)c2OC)cc2c1CN(Cc1cccc(C(=O)N(C)C)n1)CC(=O)N2C(C)C]

### Step 1. 2-Amino-4-bromo-6-methoxybenzonitrile

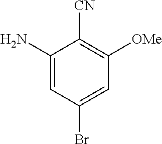 [SMILES: COc1cc(Br)cc(N)c1C#N]

To a solution of 2-amino-4-bromo-6-fluorobenzonitrile (5.0 g, 23.3 mmol) in methanol (30 mL) was added NaOMe (2.5 g, 46.5 mmol) in methanol. The reaction was stirred at 50° C. for 1 h. Then the reaction mixture was quenched with water and was extracted with DCM (10 mL×3). The combined organic phase was dried over Na₂SO₄, filtered, and concentrated under reduced pressure. The residue was purified by FCC (0% to 10% MeOH in DCM) to give the title compound (4.0 g, 17.6 mmol, 75.8% yield). LCMS calc. for C₈H₈BrN₂O [M+H]⁺: m/z=227.0/229.0; Found: 227.1/229.1.

### Step 3. Ethyl (2-amino-4-bromo-6-methoxybenzyl)glycinate

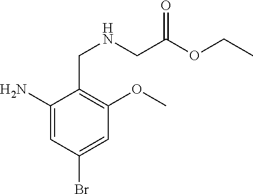 [SMILES: CCOC(=O)CNCc1c(N)cc(Br)cc1OC]

The title compound was prepared in accordance with the synthetic protocols set forth in Example 228 Steps 1 through 2 using appropriate intermediates, as well as commercial starting materials. LCMS calc. for C₁₂H₁₈BrN₂O₃[M+H]⁺: m/z=317.1/319.1; Found: 317.3/319.3.

### Step 4. tert-Butyl 8-bromo-6-methoxy-2-oxo-1,2,3,5-tetrahydro-4H-benzo[e][1,4]-diazepine-4-carboxylate

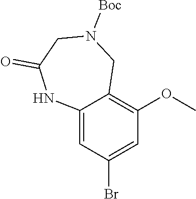 [SMILES: COc1cc(Br)cc2c1CN(C(=O)OC(C)(C)C)CC(=O)N2]

The title compound was prepared in accordance with the synthetic protocols set forth in Example 224 Steps 4 through 6 using appropriate intermediates, as well as commercial starting materials. LCMS calc. for C₁₅H₂₀BrN₂O₄[M+H]⁺: m/z=371.1/373.1; Found: 371.2/373.2.

### Step 5. 6-((8-Bromo-6-methoxy-2-oxo-1,2,3,5-tetrahydro-4H-benzo[e][1,4]diazepin-4-yl)methyl)-N,N-dimethylpicolinamide

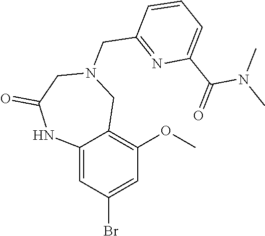 [SMILES: COc1cc(Br)cc2c1CN(Cc1cccc(C(=O)N(C)C)n1)CC(=O)N2]

To a solution of tert-butyl 8-bromo-6-methoxy-2-oxo-1,2,3,5-tetrahydro-4H-benzo[e]-[1,4]diazepine-4-carboxylate (1.1 g, 2.96 mmol) in DCM (20 mL) was added trifluoroacetic acid (10 mL). The reaction was stirred at rt for 1 h. The reaction mixture was concentrated under reduced pressure. Toluene was added and the mixture was concentrated again. The residue and 6-formyl-N,N-dimethylpicolinamide (Int. 24) (0.58 g, 3.26 mmol) were dissolved in DCM (30 mL). The reaction was stirred at rt for 30 minutes. Then sodium triacetoxyborohydride (1.88 g, 8.88 mmol) was added to the reaction mixture, and the reaction mixture was stirred for 3 hours. The reaction mixture was poured into sat. NaHCO₃ (aq) solution and was extracted by DCM/MeOH (v/v=10/1) three times. The combined organic phase was washed by brine, dried over Na₂SO₄, filtered, and concentrated under reduced pressure. The residue obtained was purified by silica gel column chromatography (0-10% MeOH/DCM) to give the title compound (0.4 g, 31.2% yield) as colorless viscous solids. LC-MS calc. for C₁₉H₂₂BrN₄O₃[M+H]⁺: m/z=433.1/435.1; Found: 433.3/435.3.

### Step 6. 6-((8-Bromo-1-isopropyl-6-methoxy-2-oxo-1,2,3,5-tetrahydro-4H-benzo[e][1,4]diazepin-4-yl)methyl)-N,N-dimethylpicolinamide

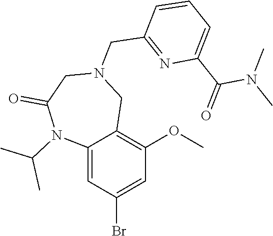 [SMILES: COc1cc(Br)cc2c1CN(Cc1cccc(C(=O)N(C)C)n1)CC(=O)N2C(C)C]

To a solution of 6-((8-bromo-6-methoxy-2-oxo-1,2,3,5-tetrahydro-4H-benzo[e][1,4]-diazepin-4-yl)methyl)-N,N-dimethylpicolinamide (200 mg, 0.46 mmol) in DMSO (4 mL) was added cesium carbonate (451.2 mg, 1.38 mmol) and 2-iodopropane (0.09 mL, 0.92 mmol). The reaction was stirred at 40° C. for 2 h. Then the reaction mixture was quenched with cold water and was extracted with EtOAc (10 mL×3). The combined organic phase was dried over Na₂SO₄, filtered and concentrated with reduced pressure. The residue obtained was purified by FCC (0% to 10% MeOH in DCM) to give the title compound (30 mg, 13.7% yield). LC-MS calc. for C₂₂H₂₈BrN₄O₃[M+H]⁺: m/z=475.1/477.1; Found: 475.2/477.2.

### Step 9. 6-((8-(4-((5-acetyl-2-(cyclopropanecarboxamido)pyridin-4-yl)amino)-3-methoxypyridin-2-yl)-1-isopropyl-6-methoxy-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 176 Steps 7 through 8 using appropriate intermediates, as well as commercial starting materials. LC-MS calc. for C₃₉H₄₅N₈O₆ [M+H]⁺: m/z=721.4; Found: 721.5.

### Example 232. 6-((8-(4-((5-Acetyl-2-(cyclopropanecarboxamido)pyridin-4-yl)amino)-3-methoxypyridin-2-yl)-6-chloro-1-ethyl-2-oxo-1,2,3,5-tetrahydro-4H-benzo[e][1,4]diazepin-4-yl)methyl)-N,N-dimethylpicolinamide

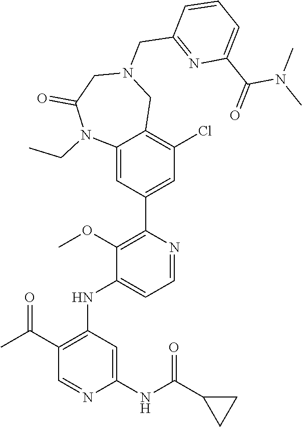 [SMILES: CCN1C(=O)CN(Cc2cccc(C(=O)N(C)C)n2)Cc2c(Cl)cc(-c3nccc(Nc4cc(NC(=O)C5CC5)ncc4C(C)=O)c3OC)cc21]

### Step 1. 4-Bromo-2-chloro-6-nitrobenzonitrile

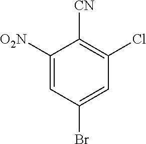 [SMILES: N#Cc1c(Cl)cc(Br)cc1[N+](=O)[O-]]

To a heterogenous mixture of copper (I) cyanide (3.56 g, 39.8 mmol) in MeCN (56 mL) at 70° C. tert-butyl nitrite (4.71 mL, 39.8 mmol) was added dropwise. The mixture was stirred at 70° C. for 20 minutes. Then 4-bromo-2-chloro-6-nitroaniline (5.00 g, 19.9 mmol) was added portion wise to the mixture at 70° C. The reaction mixture was stirred at 70° C. for 16 hours. After cooling to rt the mixture was centrifuged to remove any undissolved solids. The resulting supernatant was concentrated to dryness. The residue obtained was purified by flash column chromatography (0-50% EtOAc/heptane) to afford the title compound (1.63 g, 6.23 mmol, 31% yield) as a light orange solid. ¹H NMR (300 MHz, CDCl₃) δ 8.37 (d, J=1.8 Hz, 1H), 8.05 (d, J=1.8 Hz, 1H).

### Step 2. 2-Amino-4-bromo-6-chlorobenzamide

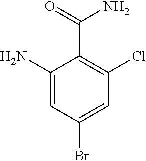 [SMILES: NC(=O)c1c(N)cc(Br)cc1Cl]

4-bromo-2-chloro-6-nitrobenzonitrile (1.63 g, 6.23 mmol) was dissolved in methanol (62 mL). Ammonium chloride (4.00 g, 74.8 mmol) was added, and the mixture was cooled to 0° C. Zinc (3.26 g, 49.9 mmol) was added portion wise and the mixture was slowly warmed to rt and stirred for an additional hour. The crude mixture was diluted with DCM (60 mL) and filtered. The filtrate was concentrated to dryness and purified by flash column chromatography (0-10% MeOH/DCM) to afford the title compound (1.02 g, 4.41 mmol, 71% yield) as an orange oil. LCMS calc. for C₇H₄BrClNO [M−H₂N]+m/z=231.92; Found: 231.88.

### Step 3. 6-((8-(4-((5-Acetyl-2-(cyclopropanecarboxamido)pyridin-4-yl)amino)-3-methoxypyridin-2-yl)-6-chloro-1-ethyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 231 Steps 2 through 10 using appropriate intermediates, as well as commercial starting materials. LCMS calc. for C₃₇H₄₀ClN₈O₅[M+H]+m/z=711.28; Found: 711.36.

### Example 233. 6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)sulfonyl)-N,N-dimethylpicolinamide

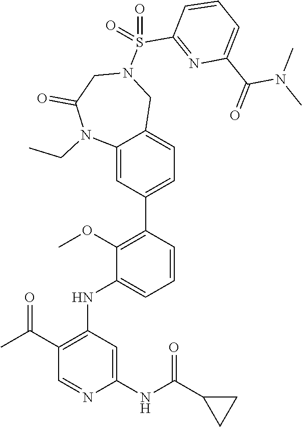 [SMILES: CCN1C(=O)CN(S(=O)(=O)c2cccc(C(=O)N(C)C)n2)Cc2ccc(-c3cccc(Nc4cc(NC(=O)C5CC5)ncc4C(C)=O)c3OC)cc21]

### Step 1. 8-Bromo-1-ethyl-1, 3, 4,5-tetrahydro-2H-benzo[e][l, 4]diazepin-2-one

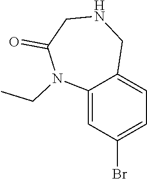

The title compound was prepared in accordance with the synthetic protocols set forth in Example 165 Steps 1 through 3 using appropriate intermediates, as well as commercial starting materials. LCMS calc. for C₁₁H₁₄BrN₂O [M+H]⁺: m/z=269.0/271.0; Found: 269.4/271.3.

### Step 2. 6-Chloro-N,N-dimethylpicolinamide

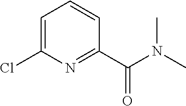

Triethylamine (0.88 mL, 6.35 mmol), 6-chloropicolinic acid (500.0 mg, 3.17 mmol) and 1-[bis(dimethylamino)methylene]-1H-1,2,3-triazolo[4,5-b]pyridinium 3-oxid hexafluorophosphate (1.45 g, 3.81 mmol) were added in DCM (5 mL) and stirred at rt for 30 min. Then 2 M dimethylamine solution in THF (0.3 mL, 4.76 mmol) was added at 0° C. The reaction mixture was stirred overnight at rt. The product mixture was diluted with water and extracted with EtOAc. The combined organic layers were dried over Na₂SO₄, filtered and concentrated under reduced pressure. The residue obtained was purified by silica chromatography (50% EtOAc/Hep) to give the title compound (580 mg, 3.14 mmol, 99% yield) as a light-yellow oil. LCMS calc. for C₈H₁₀ClN₂O [M+H]⁺: m/z=185.05; Found: 185.31.

### Step 3. 6-(Benzylthio)-N,N-dimethylpicolinamide

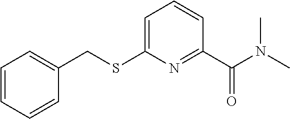 [SMILES: CN(C)C(=O)c1cccc(SCc2ccccc2)n1]

6-Chloro-N,N-dimethylpicolinamide (550.0 mg, 2.98 mmol), K₂CO₃ (823 mg, 5.96 mmol) and benzylthiol (0.4 mL, 3.57 mmol) were added in DMF (4 mL). The reaction mixture was heated to 80° C. and stirred for 16 hours. The reaction was washed with water and extracted with EtOAc (3×15 mL). The combined organic layers were dried over Na₂SO₄, filtered and concentrated under reduced pressure. The residue obtained was purified by silica gel column chromatography (50% EtOAc/Hep) to give the title compound (601 mg, 2.20 mmol, 73.9% Yield) as a yellow oil. LCMS calc. for C₁₅H₁₇N₂OS [M+H]⁺: m/z=273.11; Found: 273.39.

### Step 4. 6-(dimethylcarbamoyl)pyridine-2-sulfonyl chloride

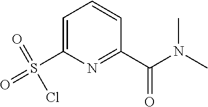

6-(Benzylthio)-N,N-dimethylpicolinamide (200.0 mg, 0.73 mmol) was dissolved in water (2 mL) and DCM (6 mL). The reaction mixture was cooled and held at 0° C. C₁₂ gas was generated by KMnO₄ and conc. HCl and was slowly purged into the reaction solution over 30 min. The reaction mixture was stirred at 0° C. for 2 h. Then, N₂ was purged for another 10 min to remove C₁₂ gas in the system. The organic layer was separated and concentrated. The crude mixture was purified by silica chromatography (50-70%, Hep/EA) to give the title compound (101 mg, 0.41 mmol, 55.3% yield) as a light-yellow oil. This compound is not stable, keep cold for long term storage. LCMS calc. for C₈H₁₀ClN₂O₃S [M+H]⁺: m/z=249.01; Found: 249.25.

### Step 5. 6-((8-Bromo-1-ethyl-2-oxo-1,2,3,5-tetrahydro-4H-benzo[e][1,4]diazepin-4-yl)sulfonyl)-N,N-dimethylpicolinamide

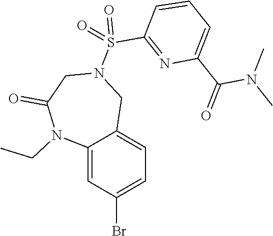 [SMILES: CCN1C(=O)CN(S(=O)(=O)c2cccc(C(=O)N(C)C)n2)Cc2ccc(Br)cc21]

8-Bromo-1-ethyl-1,3,4,5-tetrahydro-2H-benzo[e][1,4]diazepin-2-one (30 mg, 0.11 mmol) was added in 1 mL DCM followed by the addition of TEA (34 mg, 0.34 mmol) at 0° C. Then 6-(dimethylcarbamoyl)pyridine-2-sulfonyl chloride (54 mg, 0.22 mmol) in 1 mL DCM was added. The reaction was allowed to stir at rt overnight. The product mixture was washed with water. The organic layer was separated and concentrated. The residue was purified by silica chromatography (DCM/MeOH, 0-5% yield) to give the title compound (11 mg, 0.23 mmol, 20.5% yield) as a colorless film. LCMS calc. for C₁₉H₂₂BrN₄O₄S [M+H]⁺: m/z=481.05/483.05; Found: 481.28/483.29.

### Step 6. 6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)sulfonyl)-N,N-dimethylpicolinamide

[3-[[5-Acetyl-2-(cyclopropanecarbonylamino)-4-pyridinyl]amino]-2-methoxyphenyl]-boronic acid (12 mg, 0.03 mmol), [1,1′-bis(diphenylphosphino)ferrocene]dichloropalladium (II) (1.61 mg, 0.002 mmol), cesium acetate (12.7 mg, 0.07 mmol), 6-((8-bromo-1-ethyl-2-oxo-1,2,3,5-tetrahydro-4H-benzo[e][1,4]diazepin-4-yl)sulfonyl)-N,N-dimethylpicolinamide (10.6 mg, 0.02 mmol) were added in water (0.03 mL) and 1,4-dioxane (0.60 mL) under N₂. The reaction mixture was stirred at 100° C. overnight. The reaction was quenched by adding water and extracted with EtOAc (3×5 mL). The organic portion was concentrated and purified by silica chromatography (80% EtOAc/Hep then 1% MeOH/EtOAc) and prep-HPLC (MeCN/water, 0.05% Formic acid, 15-60% yield) to give the title compound (3.2 mg, 0.004 mmol, 20% yield) as the formate salt. LCMS calc. for C₃₇H₄₀N₇O₇S [M+H]⁺: m/z=726.27; Found: 726.44.

### Example 234. N-(5-Acetyl-4-((2-methoxy-3-(1-methyl-4-((6-(morpholine-4-carbonyl)pyridin-2-yl)sulfonyl)-2-oxo-2,3,4,5-tetrahydro-1H-benzo[e][1,4]diazepin-8-yl)phenyl)amino)pyridin-2-yl)cyclopropanecarboxamide

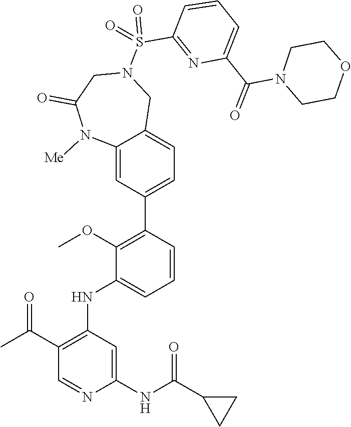 [SMILES: COc1c(Nc2cc(NC(=O)C3CC3)ncc2C(C)=O)cccc1-c1ccc2c(c1)N(C)C(=O)CN(S(=O)(=O)c1cccc(C(=O)N3CCOCC3)n1)C2]

The title compound was prepared in accordance with the synthetic protocols set forth in Example 233 Steps 1 through 6 using appropriate intermediates, as well as commercial starting materials. LCMS calc. for C₃₈H₄₀N₇O₈S [M+H]⁺: m/z=754.27; Found: 754.28. ¹H NMR (300 MHz, Methanol-d₄) δ 11.46 (s, OH), 8.76 (s, 1H), 8.23 (t, J=7.7 Hz, 1H), 8.19-8.11 (m, 1H), 7.87 (dd, J=7.6, 1.3 Hz, 1H), 7.61 (d, J=1.7 Hz, 1H), 7.56-7.46 (m, 1H), 7.45 (td, J=7.9, 1.8 Hz, 2H), 7.37 (t, J=7.7 Hz, 1H), 7.29 (d, J=8.0 Hz, 1H), 6.86 (d, J=5.1 Hz, 1H), 4.70 (s, 2H), 3.93 (s, 2H), 3.75 (s, 3H), 3.62 (t, J=4.6 Hz, 2H), 3.48 (t, J=4.8 Hz, 2H), 3.42 (s, 3H), 3.36 (s, 3H), 2.70 (s, 3H), 1.79 (ddd, J=12.6, 7.9, 4.5 Hz, 1H), 1.15-0.97 (m, 4H).

### Example 235. 6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)sulfonyl)-N,N-dimethylpicolinamide

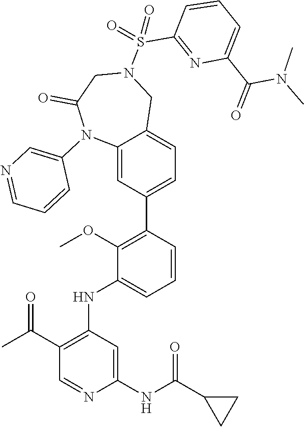 [SMILES: COc1c(Nc2cc(NC(=O)C3CC3)ncc2C(C)=O)cccc1-c1ccc2c(c1)N(c1cccnc1)C(=O)CN(S(=O)(=O)c1cccc(C(=O)N(C)C)n1)C2]

### Step 1. tert-Butyl 8-bromo-2-oxo-1-(pyridin-3-yl)-1,2,3,5-tetrahydro-4H-benzo[e][1, 4]-diazepine-4-carboxylate

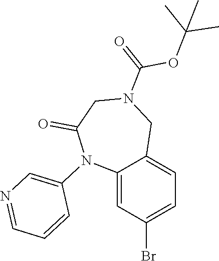 [SMILES: CC(C)(C)OC(=O)N1CC(=O)N(c2cccnc2)c2cc(Br)ccc2C1]

To a solution of tert-butyl 8-bromo-2-oxo-1,2,3,5-tetrahydro-4H-benzo[e][1,4]diazepine-4-carboxylate (100 mg, 0.29 mmol) in 1,4-dioxane (2 mL) was added 3-iodopyridine (180 mg, 0.88 mmol), potassium carbonate (81 mg, 0.59 mmol), (1S,2S)-(+)-1,2-diaminocyclohexane (7.00 mg, 0.06 mmol) and cuprous iodide (11.0 mg, 0.06 mmol) under N₂. The resulting mixture was stirred at 120° C. for 18 h. The reaction mixture was quenched by water and was extracted with ethyl acetate (20 mL×3). The combined organic layers were washed with brine, dried over sodium sulfate, filtered and concentrated under reduced pressure to obtain crude, which was purified by prep-TLC (50% EtOAc/Hexanes) to give the title compound (96.0 mg, 0.23 mmol, 78.3% yield) as a yellow solid. LCMS calc. for C₁₉H₂₁BrN₃O₃ [M+H]+. m/z=418.08; found 417.90.

### Step 2. Bromo-1-(pyridin-3-yl)-1,3,4,5-tetrahydro-2H-benzo[e][1,4]diazepin-2-one

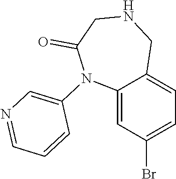 [SMILES: O=C1CNCc2ccc(Br)cc2N1c1cccnc1]

To a solution of tert-butyl 8-bromo-2-oxo-1-(pyridin-3-yl)-1,2,3,5-tetrahydro-4H-benzo[e][1,4]diazepine-4-carboxylate (96.0 mg, 0.23 mmol) in dichloromethane (1 mL) was added trifluoroacetic acid (1 mL) under N₂ at 15° C. The resulting mixture was stirred at 15° C. for 1 h. The product mixture was concentrated under reduced pressure to give the TFA salt of the title compound (73.0 mg, 0.23 mmol, quant. yield) as a yellow oil. LCMS calc. for C₁₄H₁₃BrN₃O [M+H]⁺: m/z=318.02; found 318.02.

### Step 3. 6-((8-Bromo-2-oxo-1-(pyridin-3-yl)-1,2,3,5-tetrahydro-4H-benzo[e][1,4]diazepin-4-yl)sulfonyl)-N,N-dimethylpicolinamide

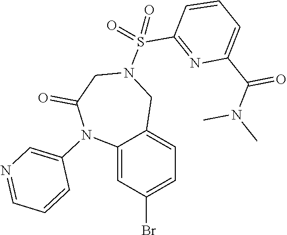 [SMILES: CN(C)C(=O)c1cccc(S(=O)(=O)N2CC(=O)N(c3cccnc3)c3cc(Br)ccc3C2)n1]

To a solution of 8-bromo-1-(pyridin-3-yl)-1,3,4,5-tetrahydro-2H-benzo[e][1,4]diazepin-2-one (24.0 mg, 0.08 mmol) in dichloromethane (2 mL) was added triethylamine (31.0 mg, 0.30 mmol) and 6-(dimethylcarbamoyl)pyridine-2-sulfonyl chloride (38.0 mg, 0.15 mmol) under N₂ at 15° C., The resulting mixture was stirred at 15° C. for 3 h. The product mixture was quenched by adding water and was extracted with dichloromethane (20 mL×3). The combined organic layers were washed with brine, dried over sodium sulfate, filtered and concentrated under reduced pressure. The residue obtained was purified by prep-HPLC on a C₁₈ column (20-70% Acetonitrile in 0.1% Trifluoroacetic acid (aq), pH=2) to give the title compound (13.0 mg, 0.03 mmol, 32.5% yield) as a white solid. LCMS calc. for C₂₂H₂₁BrN₅O₄S [M+H]⁺: m/z=530.05; Found: 530.12.

### Step 4. 6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)sulfonyl)-N,N-dimethylpicolinamide

To a solution of 6-((8-bromo-2-oxo-1-(pyridin-3-yl)-1,2,3,5-tetrahydro-4H-benzo[e]-[1,4]diazepin-4-yl)sulfonyl)-N,N-dimethylpicolinamide (13.0 mg, 0.02 mmol) in 1,4-dioxane (2.50 mL) and water (0.50 mL) was added (3-((5-acetyl-2-(cyclopropanecarboxamido)pyridin-4-yl)amino)-2-methoxyphenyl)boronic acid (12.0 mg, 0.03 mmol), potassium carbonate (11.0 mg, 0.07 mmol) and [1,1′-bis(diphenylphosphino)ferrocene]dichloropalladium (II) (2.00 mg, 2.00 mol) under N₂. The resulting mixture was stirred at 90° C. for 2 h. The product mixture was quenched by adding water and was extracted with ethyl acetate (20 mL×3). The combined organic layers were washed with brine, dried over sodium sulfate, filtered and concentrated under reduced pressure. The residue obtained was purified by prep-HPLC on a C₁₈ column (20-70% Acetonitrile in 0.1% TFA (aq), pH=2) to give the TFA salt of the title compound (4.00 mg, 5.20 mol, 21.1% yield) as a white solid. LCMS calc. for C₄₀H₃₉N₈O₇S [M+H]⁺: m/z=775.27; Found: 775.38. ¹H NMR (400 MHz, DMSO-d₆) δ 10.99 (s, 1H), 10.91 (s, 1H), 8.79 (s, 1H), 8.51-8.48 (m, 2H), 8.25 (s, 1H), 8.17 (d, J=6.8 Hz, 1H), 7.83 (s, 1H), 7.80-7.78 (m, 1H), 7.76-7.74 (m, 1H), 7.49 (d, J=3.5 Hz, 1H), 7.42 (d, J=6.4 Hz, 1H), 7.34 (s, 2H), 7.22 (s, 1H), 7.09 (d, J=6.1 Hz, 1H), 7.04 (s, 1H), 4.87 (s, 2H), 3.99 (s, 2H), 3.10 (s, 3H), 2.90 (s, 3H), 2.61 (s, 3H), 2.51 (s, 3H), 1.96 (d, J=4.9 Hz, 1H), 0.79 (ddd, J=7.0, 4.8, 2.0 Hz, 4H).

### Example 236. (R)-6-((8-(3-((5-Acetyl-2-(cyclopropanecarboxamido)pyridin-4-yl)amino)-2-methoxyphenyl)-1-ethyl-3-methyl-2-oxo-1,2,3,5-tetrahydro-4H-benzo[e][1,4]diazepin-4-yl)methyl)-N,N-dimethylpicolinamide

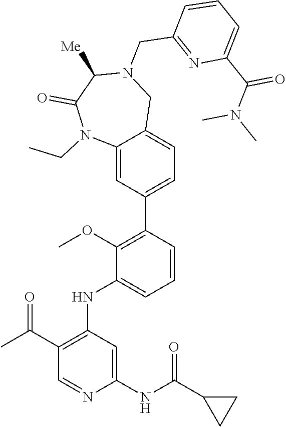 [SMILES: CCN1C(=O)[C@@H](C)N(Cc2cccc(C(=O)N(C)C)n2)Cc2ccc(-c3cccc(Nc4cc(NC(=O)C5CC5)ncc4C(C)=O)c3OC)cc21]

### Step 1. tert-Butyl (2-amino-4-bromobenzyl)-D-alaninate

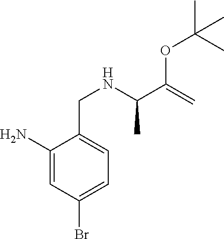 [SMILES: C=C(OC(C)(C)C)[C@@H](C)NCc1ccc(Br)cc1N]

To a solution of 2-amino-4-bromobenzaldehyde (1.00 g, 5.00 mmol) in DMSO (20 mL) was added tert-butyl D-alaninate hydrochloride (1.09 g, 6.00 mmol) and sodium triacetoxyborohydride (5.30 g, 25.0 mmol) under N₂ at 25° C. The resulting mixture was stirred at 25° C. for 40 h. The product mixture was quenched by water and was extracted with EtOAc (50 mL×3). The combined organic layers were washed with brine, dried over sodium sulfate, filtered and concentrated under reduced pressure. The residue obtained was purified by silica gel column chromatography (20% EtOAc/Hexanes) to obtain the title compound (730 mg, 2.22 mmol, 44.3% yield) as a yellow solid. LCMS calc. for C₁₄H₂₂BrN₂O₂[M+H]⁺: m/z=329.08; Found: 329.12.

### Step 2. (R)-8-Bromo-3-methyl-1,3,4,5-tetrahydro-2H-benzo[e][1,4]diazepin-2-one

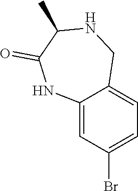

To a solution of tert-butyl (2-amino-4-bromobenzyl)-D-alaninate (730 mg, 2.22 mmol) in trifluoroacetic acid (8 mL) was added toluene (8 mL) under N₂ at 30° C. The resulting mixture was stirred at 100° C. for 1.5 h. The product mixture was concentrated under reduced pressure. The residue obtained was purified by prep-HPLC on a C₁₈ column (20-70% Acetonitrile in 0.1% Trifluoroacetic acid (aq), pH=2) to give the title compound (443 mg, 1.74 mmol, 78.3% yield) as a white solid. LCMS calc. for C₁₀H₁₂BrN₂O [M+H]⁺: m/z=255.01; Found: 255.10.

### Step 3. (R)-6-((8-Bromo-3-methyl-2-oxo-1,2,3,5-tetrahydro-4H-benzo[e][1,4]diazepin-4-yl)methyl)-N,N-dimethylpicolinamide

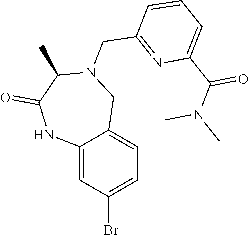 [SMILES: C[C@@H]1C(=O)Nc2cc(Br)ccc2CN1Cc1cccc(C(=O)N(C)C)n1]

To a solution of (R)-8-bromo-3-methyl-1,3,4,5-tetrahydro-2H-benzo[e][1,4]diazepin-2-one (100 mg, 0.39 mmol) in DCM (4 mL) was added 6-formyl-N,N-dimethylpicolinamide (Int. 24) (140 mg, 0.78 mmol) and 1 drop acetic acid under N₂ at 30° C. The resulting mixture was stirred at 30° C. for 2 h. Then sodium triacetoxyborohydride (416 mg, 1.96 mmol) was added to the reaction mixture under N₂ at 30° C. The resulting mixture was stirred at 30° C. for 1 h. The product mixture was quenched by water and was extracted with dichloromethane (3×100 mL). The combined organic layers were washed with brine, dried over sodium sulfate, filtered and concentrated under reduced pressure to give the crude title compound (136 mg, 0.32 mmol, 83.1% yield) as a yellow solid, which was used without further purification. LCMS calc. for C₁₉H₂₂BrN₄O₂[M+H]⁺: m/z=417.08; Found: 417.14.

### Step 4. (R)-6-((8-Bromo-1-ethyl-3-methyl-2-oxo-1,2,3,5-tetrahydro-4H-benzo[e][1,4]-diazepin-4-yl)methyl)-N,N-dimethylpicolinamide

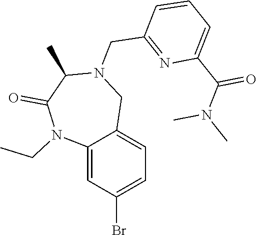 [SMILES: CCN1C(=O)[C@@H](C)N(Cc2cccc(C(=O)N(C)C)n2)Cc2ccc(Br)cc21]

To a solution of (R)-6-((8-bromo-3-methyl-2-oxo-1,2,3,5-tetrahydro-4H-benzo[e][1,4]-diazepin-4-yl)methyl)-N,N-dimethylpicolinamide (30.0 mg, 0.07 mmol) and iodoethane (34.0 mg, 0.22 mmol) in DMF (2 mL) was added cesium carbonate (141 mg, 0.43 mmol) under N₂ at 25° C. The resulting mixture was stirred at 80° C. for 2 h. The product mixture was quenched by water and was extracted with ethyl acetate (20 mL×3). The combined organic layers were washed with brine, dried over sodium sulfate, filtered and concentrated under reduced pressure to give the crude title compound (25.0 mg, 0.06 mmol, 78.1% yield) as a yellow oil, which was used without further purification. LCMS calc. for C₂₁H₂₆BrN₄O₂[M+H]⁺: m/z=445.12; Found: 445.10.

### Step 5. (R)-6-((8-(3-((5-acetyl-2-(cyclopropanecarboxamido)pyridin-4-yl)amino)-2-methoxyphenyl)-1-ethyl-3-methyl-2-oxo-1,2,3,5-tetrahydro-4H-benzo[e][1,4]diazepin-4-yl)methyl)-N,N-dimethylpicolinamide

To a solution of (R)-6-((8-bromo-1-ethyl-3-methyl-2-oxo-1,2,3,5-tetrahydro-4H-benzo[e][1,4]diazepin-4-yl)methyl)-N,N-dimethylpicolinamide (25.0 mg, 0.06 mmol) in 1,4-dioxane (2.50 mL) and water (0.50 mL) was added [3-[[5-acetyl-2-(cyclopropanecarbonylamino)-4-pyridinyl]amino]-2-methoxyphenyl]boronic acid (62.0 mg, 0.17 mmol), potassium carbonate (24.0 mg, 0.17 mmol) and [1,1′-bis(diphenylphosphino)ferrocene]dichloropalladium (II) (4.00 mg, 0.01 mmol) under N₂. The resulting mixture was stirred at 90° C. for 2 h. The reaction mixture was quenched by water and was extracted with EtOAc (20 mL×3). The combined organic layers were washed with brine, dried over sodium sulfate, filtered and concentrated under reduced pressure. The residue obtained was purified by prep-HPLC on a C₁₈ column (20-70% Acetonitrile in 0.1% Trifluoroacetic acid (aq), pH=2) to give the title compound (19.0 mg, 0.03 mmol, 49.1% yield) as a yellow solid. LCMS calc. for C₃₉H₄₄N₇O₅ [M+H]⁺: m/z=690.33; Found: 690.60. ¹H NMR (400 MHz, DMSO-d₆) δ 11.01 (s, 1H), 10.92 (s, 1H), 8.81 (s, 1H), 8.02 (s, 1H), 7.91 (d, J=7.8 Hz, 1H), 7.62-7.55 (m, 2H), 7.47-7.40 (m, 3H), 7.32-7.25 (m, 3H), 4.13 (dd, J=13.9, 7.1 Hz, 1H), 3.65 (q, J=9.1, 8.0 Hz, 4H), 3.49 (d, J=13.4 Hz, 1H), 3.32 (s, 3H), 3.09 (d, J=6.3 Hz, 1H), 2.99 (s, 3H), 2.95 (s, 3H), 2.62 (s, 3H), 2.02-1.97 (m, 1H), 1.15 (d, J=6.2 Hz, 3H), 1.02 (t, J=7.1 Hz, 3H), 0.77 (dd, J=6.2, 4.0 Hz, 4H).

### Example 237. (S)-6-((8-(3-((5-Acetyl-2-(cyclopropanecarboxamido)pyridin-4-yl)amino)-2-methoxyphenyl)-1-ethyl-3-methyl-2-oxo-1,2,3,5-tetrahydro-4H-benzo[e][1,4]diazepin-4-yl)methyl)-N,N-dimethylpicolinamide

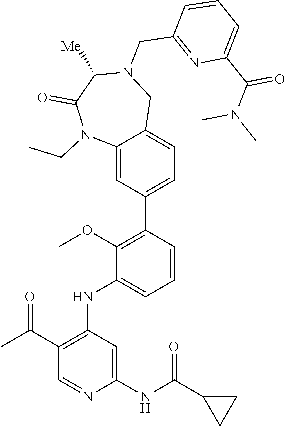 [SMILES: CCN1C(=O)[C@H](C)N(Cc2cccc(C(=O)N(C)C)n2)Cc2ccc(-c3cccc(Nc4cc(NC(=O)C5CC5)ncc4C(C)=O)c3OC)cc21]

The title compound was prepared in accordance with the synthetic protocols set forth in Example 236 using appropriate intermediates, as well as commercial starting materials. LCMS calc. for C₃₉H₄₄N₇O₅ [M+H]⁺: m/z=690.33; Found: 690.57. ¹H NMR (400 MHz, DMSO-d₆) δ 11.01 (s, 1H), 10.92 (s, 1H), 8.81 (s, 1H), 8.02 (s, 1H), 7.91 (d, J=7.7 Hz, 1H), 7.62-7.56 (m, 2H), 7.47-7.40 (m, 3H), 7.31-7.24 (m, 3H), 4.16-4.11 (m, 1H), 3.69-3.58 (m, 4H), 3.49 (d, J=13.4 Hz, 1H), 3.32 (s, 3H), 3.09 (d, J=6.3 Hz, 1H), 2.99 (s, 3H), 2.94 (s, 3H), 2.62 (s, 3H), 2.02-1.97 (m, 1H), 1.15 (d, J=6.2 Hz, 3H), 1.02 (t, J=7.0 Hz, 3H), 0.77 (dq, J=5.9, 3.3 Hz, 4H).

### Example 238. 6-((8-(3-((5-Acetyl-2-(cyclopropanecarboxamido)pyridin-4-yl)amino)-2-methoxyphenyl)-1-ethyl-5-methyl-2-oxo-1,2,3,5-tetrahydro-4H-benzo[e][1,4]diazepin-4-yl)methyl)-N,N-dimethylpicolinamide

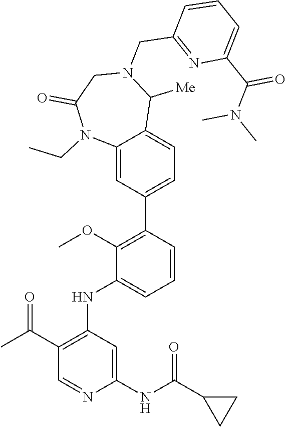 [SMILES: CCN1C(=O)CN(Cc2cccc(C(=O)N(C)C)n2)C(C)c2ccc(-c3cccc(Nc4cc(NC(=O)C5CC5)ncc4C(C)=O)c3OC)cc21]

### Step 1. 6-((8-Bromo-5-methyl-2-oxo-1,2,3,5-tetrahydro-4H-benzo[e][1,4]diazepin-4-yl)methyl)-N,N-dimethylpicolinamide

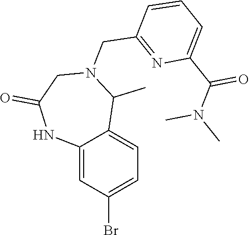 [SMILES: CC1c2ccc(Br)cc2NC(=O)CN1Cc1cccc(C(=O)N(C)C)n1]

The title compound was prepared in accordance with the synthetic protocols set forth in Int. 25 Steps 1 through 2 and Int. 26 Step 1 using appropriate intermediates, as well as commercial starting materials. LCMS calc. for C₁₉H₂₂BrN₄O₂[M+H]⁺: m/z=417.09; Found: 417.19.

### Step 2. 6-((8-(3-((5-acetyl-2-(cyclopropanecarboxamido)pyridin-4-yl)amino)-2-methoxyphenyl)-1-ethyl-5-methyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 36 Steps 1 through 2 using appropriate intermediates, as well as commercial starting materials. LCMS calc. for C₃₉H₄₄N₇O₅ [M+H]⁺: m/z=690.3; Found: 690.5. ¹H NMR (400 MHz, DMSO-d₆) δ 11.06 (s, 2H), 8.85 (s, 1H), 8.01-7.95 (m, 2H), 7.68 (s, 1H), 7.64-7.55 (m, 3H), 7.51 (dd, J=6.7, 2.9 Hz, 2H), 7.31 (dd, J=6.9, 4.3 Hz, 2H), 4.12 (dd, J=13.9, 7.0 Hz, 3H), 3.83 (dd, J=14.1, 7.1 Hz, 2H), 3.38 (s, 3H), 3.22 (dd, J=23.8, 10.9 Hz, 2H), 3.01 (s, 3H), 2.91 (s, 3H), 2.66 (d, J=6.5 Hz, 3H), 2.00 (dd, J=10.9, 6.1 Hz, 1H), 1.70 (s, 3H), 1.15 (t, J=7.1 Hz, 3H), 0.84-0.77 (m, 4H).

### Example 239. 6-((8-(3-((2-(Cyclopropanecarboxamido)-5-(2-fluoroacetyl)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

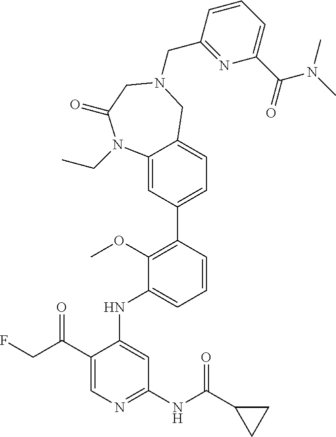 [SMILES: CCN1C(=O)CN(Cc2cccc(C(=O)N(C)C)n2)Cc2ccc(-c3cccc(Nc4cc(NC(=O)C5CC5)ncc4C(=O)CF)c3OC)cc21]

### Step 1. 1-(4-((3-Bromo-2-methoxyphenyl)amino)-6-chloropyridin-3-yl)-2-fluoroethan-1-one

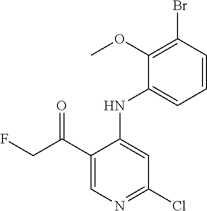 [SMILES: COc1c(Br)cccc1Nc1cc(Cl)ncc1C(=O)CF]

To 1-(4-((3-bromo-2-methoxyphenyl)amino)-6-chloropyridin-3-yl)ethan-1-one (100 mg, 0.28 mmol) in toluene (2 mL) was added triethylamine (196 μL, 1.41 mmol) and TMSOTf (254 μL, 1.41 mmol) and the reaction mixture was stirred at 80° C. for 90 minutes. The reaction was cooled and the top layer was transferred to another vial and directly condensed. The residue was taken up in MeCN (2 mL), Selectfluor (199 mg, 0.56 mmol) was added, and the reaction was stirred at rt for 1 h. Water and ethyl acetate were added to the reaction and after 10 minutes of stirring and the organic layer was dried over Na₂SO₄, condensed, and purified by SiO₂ FCC (0-30% EtOAc/heptane) to afford the title compound as a yellow solid (90 mg, 86% yield). LC-MS calc. for C₁₄H₁₂BrClFN₂O₂ [M+H]⁺: m/z=375.0/373.0/377.0; Found: 375.2/373.0/377.2.

### Step 2. N-(4-((3-Bromo-2-methoxyphenyl)amino)-5-(2-fluoroacetyl)pyridin-2-yl)cyclopropanecarboxamide

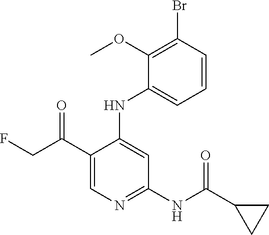 [SMILES: COc1c(Br)cccc1Nc1cc(NC(=O)C2CC2)ncc1C(=O)CF]

The title compound was prepared in accordance with the protocols set forth in Intermediate 18 using the appropriate intermediates. LC-MS calc. for C₁₈H₁₈BrFN₃O₃[M+H]⁺: m/z=422.0/424.0; Found: 422.3/424.3.

### Step 3. 6-((8-(3-((2-(Cyclopropanecarboxamido)-5-(2-fluoroacetyl)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

The title compound was prepared in accordance with the protocols set forth in Example 14 Step 2 using the appropriate intermediates. LC-MS calc. for C₃₈H₄₁FN₇O₅[M+H]⁺: m/z=694.3; Found: 694.7.

### Example 240. 6-((8-(3-((2-(Cyclopropanecarboxamido)-5-(2,2-difluoroacetyl)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

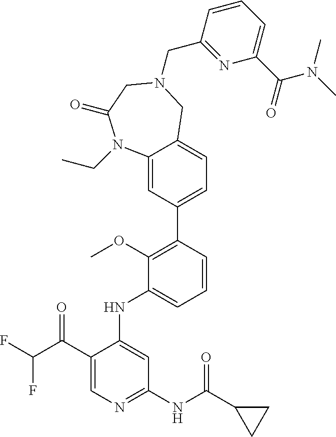 [SMILES: CCN1C(=O)CN(Cc2cccc(C(=O)N(C)C)n2)Cc2ccc(-c3cccc(Nc4cc(NC(=O)C5CC5)ncc4C(=O)C(F)F)c3OC)cc21]

### Step 1. 1-(4,6-Dichloropyridin-3-yl)-2,2-difluoroethan-1-ol

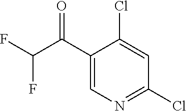 [SMILES: O=C(c1cnc(Cl)cc1Cl)C(F)F]

To a stirring solution of 4,6-dichloronicotinaldehyde (700 mg, 3.98 mmol and (bromo-difluoromethyl)trimethylsilane (1.21 g, 5.97 mmol) in DMF (14 mL) was added triphenylphosphine (1.56 g, 5.97 mmol) and the resulting mixture was stirred at rt. After 2 hours, it was cooled to 0° C., KOH (8.0 mL, 11.9 mmol, 1.5 M (aq)) was added dropwise. The reaction was stirred at 0° C. for 1 hour, quenched with sat. NH₄Cl (aq), and extracted with MTBE 2×. The combined organics were dried over Na₂SO₄, condensed, and purified by SiO₂ FCC (0-30% MTBE/heptane) to afford the title compound (740 mg, 82%) as a white solid. LC-MS calc. for C₇H₆Cl₂F₂NO [M+H]⁺: m/z=228.0/230.0; Found 228.2/230.3.

### Step 2. 1-(4,6-Dichloropyridin-3-yl)-2,2-difluoroethan-1-one

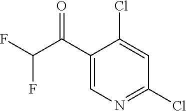 [SMILES: O=C(c1cnc(Cl)cc1Cl)C(F)F]

To a stirring solution of 1-(4,6-dichloropyridin-3-yl)-2,2-difluoroethan-1-ol (700 mg, 3.1 mmol) in DCM (20 mL) at 0° C. was added Dess-Martin periodinane (3.91 g, 9.2 mmol) and the reaction mixture was allowed to warm to room temperature over 3 hours. The reaction was quenched with sat. NaHCO₃ (aq) and extracted with DCM 2×. The combined extracts were washed with brine, dried over Na₂SO₄, condensed, and purified by silica gel chromatography (0-10% EtOAc/heptane) to afford the title compound (680 mg, 98% yield). LC-MS calc. for C₇H₄Cl₂F₂NO [M+H]⁺: m/z=226.0; Found: 226.3.

### Step 3. 1-(4-((3-Bromo-2-methoxyphenyl)amino)-6-chloropyridin-3-yl)-2,2-difluoroethan-1-one

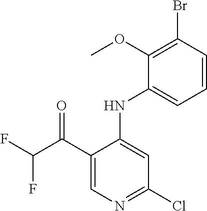 [SMILES: COc1c(Br)cccc1Nc1cc(Cl)ncc1C(=O)C(F)F]

The title compound was prepared in accordance with the protocols set forth in Int. 3 Step 1 using the appropriate intermediates and commercial starting materials. LC-MS calc. for C₁₄H₁₁BrClF₂N₂O₂ [M+H]⁺: m/z=391.0; Found: 391.2.

### Step 4. N-(4-((3-Bromo-2-methoxyphenyl)amino)-5-(2,2-difluoroacetyl)pyridin-2-yl)cyclopropanecarboxamide

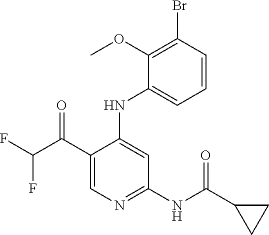 [SMILES: COc1c(Br)cccc1Nc1cc(NC(=O)C2CC2)ncc1C(=O)C(F)F]

The title compound was prepared in accordance with the protocols set forth in Int. 18 using the appropriate intermediates and commercial starting materials. LC-MS calc. for C₁₈H₁₇BrF₂N₃O₃ [M+H]⁺: m/z=440.0; Found: 440.3.

### Step 5. 6-((8-(3-((2-(Cyclopropanecarboxamido)-5-(2,2-difluoroacetyl)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

The title compound was prepared as a TFA salt using procedures analogous to Example 14 Step 2 using the appropriate intermediates. LC-MS calc. for C₃₈H₄₀F₂N₇O₅[M+H]⁺: m/z=712.3; Found: 712.6.

### Example 241. 6-((8-(3-((5-Acetyl-2-(cyclopropanecarboxamido)pyrimidin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

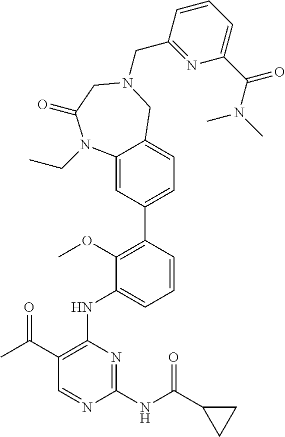 [SMILES: CCN1C(=O)CN(Cc2cccc(C(=O)N(C)C)n2)Cc2ccc(-c3cccc(Nc4nc(NC(=O)C5CC5)ncc4C(C)=O)c3OC)cc21]

### Step 1. 1-(4-((3-Bromo-2-methoxyphenyl)amino)-2-chloropyrimidin-5-yl)ethan-1-one

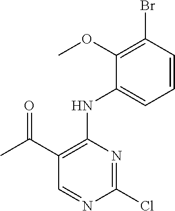 [SMILES: COc1c(Br)cccc1Nc1nc(Cl)ncc1C(C)=O]

A mixture of 1-(2,4-dichloropyrimidin-5-yl)ethan-1-one (900 mg, 4.71 mmol) and 3-bromo-2-methoxyaniline (1.14 g, 5.65 mmol) was dissolved in DCM (10 mL) and tert-butanol (10 mL). The mixture was cooled to 0° C. and zinc chloride (642 mg, 4.71 mmol) was added. The reaction was stirred at 0° C. for 2 h. The product mixture was quenched with sat. NH₄Cl (aq) solution, diluted with brine (100 mL), and was extracted with DCM 3 times. The combined organic layers were dried with Na₂SO₄, filtered and concentrated under reduced pressure. The residue obtained was purified by silica gel column chromatography (2-30%, EtOAc/Hexanes) to afford the title compound (710 mg, 1.99 mmol, 42.3% yield). LCMS calc. for C₁₃H₁₂BrClN₃O₂[M+H]⁺: m/z=358.0/356.0/360.0; Found: 358.3/356.3/360.3.

### Step 2. N-[5-Acetyl-4-(3-bromo-2-methoxyanilino)pyrimidin-2-yl]cyclopropanecarboxamide

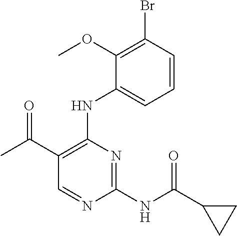 [SMILES: COc1c(Br)cccc1Nc1nc(NC(=O)C2CC2)ncc1C(C)=O]

To a solution of 1-(4-((3-bromo-2-methoxyphenyl)amino)-2-chloropyrimidin-5-yl)ethan-1-one (200 mg, 0.56 mmol), 9,9-dimethyl-4,5-bis(diphenylphosphino)xanthene (23.4 mg, 0.04 mmol) cyclopropanecarboxamide (3.5 mg, 0.04 mmol), CsOAc (23.3 mg, 0.12 mmol) in 1,4-dioxane (3 mL) was added Pd₂(dba)₃ (37.0 mg, 0.04 mmol). The tube was sealed, placed under vacuum and backfilled with nitrogen gas. The reaction was stirred at 60° C. for 2 h. The product mixture was diluted with water and extracted with DCM 3 times. The combined organic layers were dried with Na₂SO₄, filtered and concentrated under reduced pressure. The residue obtained was purified by silica gel column chromatography (1-5% MeOH:DCM) then Prep HPLC (30-65% MeCN in H₂O with 0.05% TFA) to afford the title compound (125 mg, 0.31 mmol, 55.0% yield). LCMS calc. for C₁₇H₁₈BrN₄O₃[M+H]⁺: m/z=405.1/407.1; Found: 405.2/407.2.

### Step 3. 6-((8-(3-((5-Acetyl-2-(cyclopropanecarboxamido)pyrimidin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

To a solution of 6-((8-bromo-1-ethyl-2-oxo-1,2,3,5-tetrahydro-4H-benzo[e][1,4]diazepin-4-yl)methyl)-N,N-dimethylpicolinamide (22.4 mg, 0.047 mmol) N-[5-acetyl-4-(3-bromo-2-methoxyanilino)pyrimidin-2-yl]cyclopropanecarboxamide (19 mg, 0.047 mmol) and CsOAc (27.0 mg, 0.14 mmol) in 1,4-dioxane (1.6 mL) and water (0.40 mL) was added Pd(dppf)Cl₂ (3.4 mg, 0.0047 mmol). The reaction mixture was sealed, placed under vacuum and backfilled with nitrogen 5 times. The mixture was stirred at 60° C. for 6 h. The product mixture was cooled to rt and diluted with acetonitrile. The crude mixture was filtered, concentrated, dissolved in DMSO, and purified by prep-HPLC (MeCN/water 20% to 45% with 0.05% TFA) to the title compound (3.3 mg, 0.0049 mmol, 10.4% yield) as a white solid. LCMS calc. for C₃₇H₄₁N₈O₅ [M+H]⁺: m/z=677.3; Found: 677.5.

### Example 242. 6-((8-(3-((3-Acetyl-6-(cyclopropanecarboxamido)pyridazin-4-yl)amino)-2-methoxyphenyl)-1-methyl-2-oxo-1,2,3,5-tetrahydro-4H-benzo[e][1,4]diazepin-4-yl)methyl)-N,N-dimethylpicolinamide

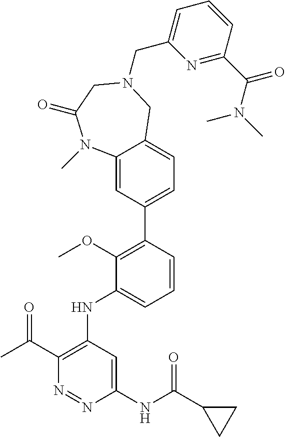 [SMILES: COc1c(Nc2cc(NC(=O)C3CC3)nnc2C(C)=O)cccc1-c1ccc2c(c1)N(C)C(=O)CN(Cc1cccc(C(=O)N(C)C)n1)C2]

### Step 1. 4,6-Dichloro-N-methoxy-N-methylpyridazine-3-carboxamide

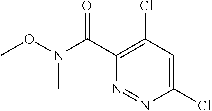

To a solution of lithium 4,6-dichloropyridazine-3-carboxylate (1.50 g, 7.54 mmol) in DCM (15 mL) was added N-methoxymethanamine; hydrochloride (1.839 g, 18.85 mmol) and pyridine (3.05 mL, 37.71 mmol) at 0° C. under N₂. Then phosphorus oxychloride (3.51 mL, 37.71 mmol) was added dropwise to the mixture at 0° C. The resulting mixture was stirred at 25° C. for 3 h. The product mixture was poured into water and extracted with DCM (45 mL). The organic layer was washed with sat. NaHCO₃ (aq) (15 mL) and sat. brine (aq) (10 mL), dried over Na₂SO₄, filtered, and concentrated under reduced pressure. The residue obtained was purified by trituration with hexane:ethyl acetate=10:1-1:1 to afford the title compound (1.5 g, 6.35 mmol, 84.2% yield) as a white solid. LCMS calc. for C₇H₈C₁₂N₃O₂ [M+H]⁺: m/z=236.0; Found: 236.1.

### Step 2. 6-Chloro-4-((2,4-dimethoxybenzyl)amino)-N-methoxy-N-methylpyridazine-3-carboxamide

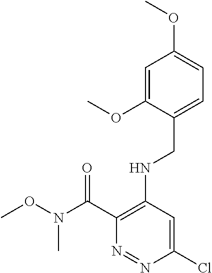 [SMILES: COc1ccc(CNc2cc(Cl)nnc2C(=O)N(C)OC)c(OC)c1]

4,6-Dichloro-N-methoxy-N-methylpyridazine-3-carboxamide (1.20 g, 5.08 mmol) and 2,4-dimethoxybenzylamine (2.29 mL, 15.3 mmol) were added to DMF (10 mL). The reaction mixture was heated to 25° C. for 16 hours. The product mixture was poured into ethyl acetate (10 mL) and water (10 mL). The organic phase was washed with sat. brine (aq) (10 mL) and concentrated under reduced pressure. The residue obtained was purified by silica gel column chromatography (16-33% EtOAc/Hexanes) to give the title compound (650 mg, 1.77 mmol, 34.8% yield) as a yellow oil. LCMS calc. for C₁₆H₂₀ClN₄O₄[M+H]⁺: m/z=367.1; Found: 367.3.

### Step 3. 1-(6-Chloro-4-((2,4-dimethoxybenzyl)amino)pyridazin-3-yl)ethan-1-one

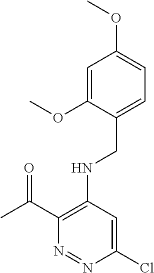 [SMILES: COc1ccc(CNc2cc(Cl)nnc2C(C)=O)c(OC)c1]

To a solution of 6-chloro-4-((2,4-dimethoxybenzyl)amino)-N-methoxy-N-methyl-pyridazine-3-carboxamide (675 mg, 1.84 mmol) in THF (10 mL) was added dropwise methylmagnesium bromide (1.23 mL, 3.68 mmol, 3.2 M in 2-MeTHF) at −70° C. The reaction mixture was stirred at −70° C. for 1.5 h. The mixture was quenched with sat. NH₄Cl (aq) solution (20 mL) and warmed to rt. The aqueous layer was extracted with EtOAc (30 mL). The organic layer was dried over Na₂SO₄, filtered, and concentrated under reduced pressure. The residue obtained was purified by basic silica gel column chromatography (16-50% EtOAc/Hexanes) to give the title compound (430 mg, 1.33 mmol, 72.6% yield) as a yellow solid. LCMS calc. for C₁₅H₁₇ClN₃O₃ [M+H]⁺: m/z=322.1; Found: 322.24.

### Step 5. N-(6-Acetyl-5-((2,4-dimethoxybenzyl)amino)pyridazin-3-yl)cyclopropanecarboxamide

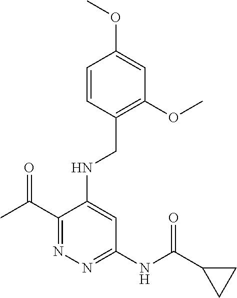 [SMILES: COc1ccc(CNc2cc(NC(=O)C3CC3)nnc2C(C)=O)c(OC)c1]

The title compound was prepared in accordance with the synthetic protocols set forth in Int. 3 Step 2 using appropriate intermediates, as well as commercial starting materials. LCMS calc. for C₁₉H₂₃N₄O₄ [M+H]⁺: m/z=371.2; Found: 371.4.

### Step 6. N-(6-Acetyl-5-aminopyridazin-3-yl)cyclopropanecarboxamide

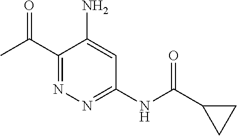 [SMILES: CC(=O)c1nnc(NC(=O)C2CC2)cc1N]

To a stirred solution of N-[6-acetyl-5-[(2,4-dimethoxyphenyl)methylamino]pyridazin-3-yl]cyclopropanecarboxamide (900 mg, 2.43 mmol) in DCM (10 mL) was added TFA (3.0 mL) at 0° C. The reaction mixture was warmed to 25° C. and stirred at 25° C. for 1 h. The product mixture was evaporated under reduced pressure afford the TFA salt of the title compound (450 mg, 2.04 mmol, 84.1% yield) as a yellow solid. LCMS calc. for C₁₀H₁₃N₄O₂ [M+H]⁺: m/z=221.1; Found: 221.17.

### Step 7. N-[6-Acetyl-5-(3-bromo-2-methoxyanilino)pyridazin-3-yl]cyclopropanecarboxamide

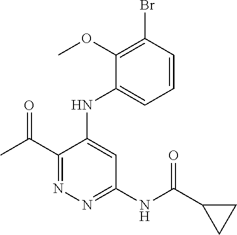 [SMILES: COc1c(Br)cccc1Nc1cc(NC(=O)C2CC2)nnc1C(C)=O]

To a solution of N-(6-acetyl-5-aminopyridazin-3-yl)cyclopropanecarboxamide (450 mg, 2.04 mmol) in 1,4-dioxane (20 mL) was added 2,6-dibromanisol (543 mg, 2.04 mmol), tris(dibenzylideneacetone)dipalladium (374 mg, 0.41 mmol), Xantphos (472 mg, 0.82 mmol) and cesium carbonate (1.99 mg, 6.13 mmol). The resulting mixture was purged with N₂ three times, and the mixture was stirred at 110° C. for 3 h. The mixture was concentrated to give a residue. The residue was purified by silica gel column chromatography to give the title compound (410 mg, 1.01 mmol, 49.5% yield) as a yellow solid. LCMS calc. for C₁₇H₁₈BrN₄O₃[M+H]⁺: m/z=405.06; Found: 405.25.

### Step 8. 6-((8-(3-((3-Acetyl-6-(cyclopropanecarboxamido)pyridazin-4-yl)amino)-2-methoxyphenyl)-1-methyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 36 Step 2 using appropriate intermediates, as well as commercial starting materials. LCMS calc. for C₃₆H₃₉N₈O₅ [M+H]⁺: m/z=663.3; Found: 663.4. ¹H NMR (400 MHz, DMSO-d₆) δ 11.54 (s, 1H), 10.54 (s, 1H), 8.45 (s, 1H), 8.14 (s, 1H), 7.93 (t, J=7.7 Hz, 2H), 7.59-7.56 (m, 2H), 7.49-7.45 (m, 2H), 7.44 (s, 1H), 7.32-7.31 (m, 1H), 3.86 (s, 2H), 3.68 (s, 2H), 3.36 (s, 3H), 3.08 (s, 2H), 3.01 (s, 3H), 2.94 (s, 3H), 2.79 (s, 3H), 2.35-2.31 (m, 3H), 2.10 (dd, J=5.2, 3.3 Hz, 1H), 0.87-0.81 (m, 4H).

### Example 243. 6-((8-(3-((2-(Cyclopropanecarboxamido)-5-oxo-5,6-dihydro-1,6-naphthyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

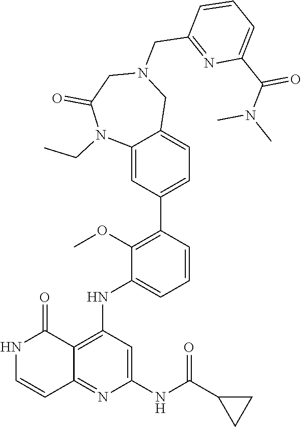 [SMILES: CCN1C(=O)CN(Cc2cccc(C(=O)N(C)C)n2)Cc2ccc(-c3cccc(Nc4cc(NC(=O)C5CC5)nc5cc[nH]c(=O)c45)c3OC)cc21]

### Step 1. 2,4-Dichloro-1, 6-naphthyridin-5(6H)-one

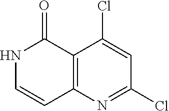 [SMILES: O=c1[nH]ccc2nc(Cl)cc(Cl)c12]

To a solution of ethyl 4,6-dichloro-2-methylnicotinate (1.00 g, 4.27 mmol) in tert-butanol (10 mL) were added 1,3,5-triazine (692 mg, 8.54 mmol) and potassium tert-butoxide (958 mg, 8.54 mmol). After stirring at 90° C. for 1 h, the mixture was cooled to 25° C. and evaporated under reduced pressure. The resulting residue was diluted with water and then acidified to pH=5 using an aqueous 2 M HCl solution. The mixture was diluted with water (5 mL) and ethyl acetate (5 mL) and was filtered through celite to collect the dark precipitate. The layers of the filtrate were separated, and the aqueous layer was extracted with ethyl acetate (30 mL). The combined organic layers were dried over sodium sulfate, filtered, combined with the previously obtained dark precipitate, and concentrated. The residue was then purified by silica gel column chromatography (0-70% EtOAc/Hexanes) to afford the title compound (820 mg, 3.81 mmol, 89.26% yield) as a white solid. LCMS calc. for C₈H₅C₁₂N₂O [M+H]⁺: m/z=215.0; Found: 215.1.

### Step 2. 4-(3-Bromo-2-methoxyanilino)-2-chloro-6H-1,6-naphthyridin-5-one

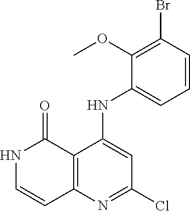 [SMILES: COc1c(Br)cccc1Nc1cc(Cl)nc2cc[nH]c(=O)c12]

To a solution of 2,4-dichloro-1,6-naphthyridin-5(6H)-one (100 mg, 0.47 mmol) in THF (3 mL) was added 3-bromo-2-methoxyaniline (94.0 mg, 0.47 mmol). The mixture was purged with N₂ three times and then lithium bis(trimethylsilyl)amide (1.4 mL, 1.4 mmol, 1.3 M in THF) was added to the mixture at 0° C. The resulting mixture was stirred at 25° C. for 1 h. The reaction mixture was poured into sat. NH₄Cl (aq) (10 mL) and was extracted with DCM (30 mL). The combined organic layers were washed with brine, dried over sodium sulfate, and concentrated under reduced pressure to give a residue. The residue was purified by trituration with hexane:ethyl acetate=5:1 to afford the title compound (56 mg, 0.147 mmol, 31.6% yield) as a light yellow solid. LCMS calc. for C₁₅H₁₂BrClN₃O₂ [M+H]⁺: m/z=380.0; Found: 380.2.

### Step 3. N-(4-((3-Bromo-2-methoxyphenyl)amino)-5-oxo-5,6-dihydro-1, 6-naphthyridin-2-yl)cyclopropanecarboxamide

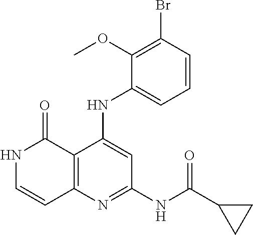 [SMILES: COc1c(Br)cccc1Nc1cc(NC(=O)C2CC2)nc2cc[nH]c(=O)c12]

The title compound was prepared in accordance with the synthetic protocols set forth in Int. 3 Step 2 using appropriate intermediates, as well as commercial starting materials. LCMS calc. for C₁₉H₁₈BrN₄O₃[M+H]⁺: m/z=429.06; Found: 429.05.

### Step 5. 6-((8-(3-((2-(Cyclopropanecarboxamido)-5-oxo-5,6-dihydro-1, 6-naphthyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide

The title compound was prepared in accordance with the synthetic protocols set forth in Example 36 Step 2 using appropriate intermediates, as well as commercial starting materials. LCMS calc. for C₃₉H₄₁N₈O₅ [M+H]⁺: m/z=701.3; Found: 701.2.

### Example 244. 6-((8-(3-((5-acetyl-2-(2-cyclopropylacetamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

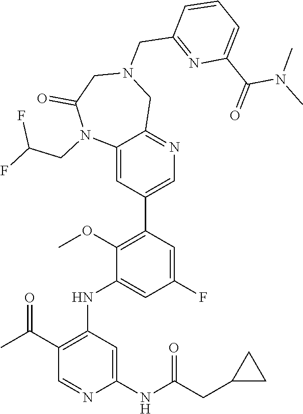 [SMILES: COc1c(Nc2cc(NC(=O)CC3CC3)ncc2C(C)=O)cc(F)cc1-c1cnc2c(c1)N(CC(F)F)C(=O)CN(Cc1cccc(C(=O)N(C)C)n1)C2]

### Step 1. tert-butyl (5-acetyl-4-((3-(1-(2, 2-difluoroethyl)-4-((6-(dimethylcarbamoyl)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)carbamate

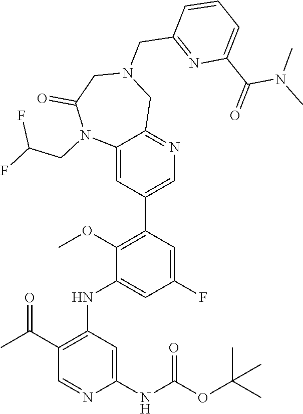 [SMILES: COc1c(Nc2cc(NC(=O)OC(C)(C)C)ncc2C(C)=O)cc(F)cc1-c1cnc2c(c1)N(CC(F)F)C(=O)CN(Cc1cccc(C(=O)N(C)C)n1)C2]

The title compound was prepared in accordance with the synthetic protocols set forth in Example 103 Steps 2-3 using the appropriate intermediates and commercial materials. LC-MS calc. for C₃₈H₄₂F₃N₈O₆[M+H]⁺: m/z=763.3; Found: 763.3.

### Step 2. 6-((8-(3-((5-acetyl-2-aminopyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

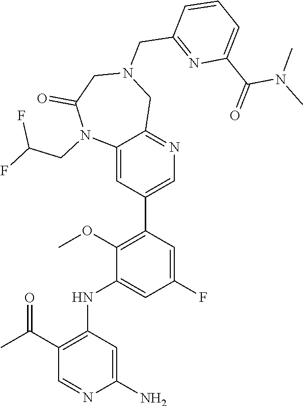 [SMILES: COc1c(Nc2cc(N)ncc2C(C)=O)cc(F)cc1-c1cnc2c(c1)N(CC(F)F)C(=O)CN(Cc1cccc(C(=O)N(C)C)n1)C2]

The title compound was prepared as a free base in accordance with the synthetic protocols set forth in Example 175 Step 5 using the appropriate intermediates. LC-MS calc. for C₃₃H₃₄F₃N₈O₄ [M+H]⁺: m/z=663.3; Found: 663.3.

### Step 3: 6-((8-(3-((5-acetyl-2-(2-cyclopropylacetamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

To a stirring solution of 6-((8-(3-((5-acetyl-2-aminopyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide (10.8 mg, 0.016 mmol) and 2-cyclopropylacetic acid (1.9 mg, 0.019 mmol) in pyridine (0.5 mL) at 0° C. was added phosphorus oxychloride (3 μL, 0.033 mmol). The reaction was stirred at 0° C. for 1 hour before being quenched with water and TFA, diluted in MeOH, and purified by prep-LCMS (CSH-C18, 20.2-40.2% MeCN in 0.2% TFA (aq)). The purified fractions were lyophilized to afford the title compound (2.1 mg, 12% yield) as a TFA salt. LC-MS calc. for C₃₈H₄₀F₃N₈O₅[M+H]⁺: m/z=745.3; Found: 745.3.

### Examples 245-247

Examples 245-247 are shown below in Table 22 and were prepared in accordance with the synthetic protocols set forth in Example 244 Step 3 using appropriate intermediates, as well as commercial starting materials. The following compounds are TFA salts unless otherwise noted.

TABLE 22
Example 245-247

| | | | Analytical Data |
|---|---|---|---|
| | | | (LCMS and/or |
| Ex. | Name | Structure | NMR) |
| 245 | 6-((8-(3-((5-acetyl-2- ((1R,2R)-2- (trifluoromethyl) cyclopropane-1- carboxamido)pyridin- 4-yl)amino)-5-fluoro-2- methoxyphenyl)-1-(2,2- difluoroethyl)-2-oxo-1,2,3,5- tetrahydro-4H-pyrido[3,2- e][1,4]diazepin-4- yl)methyl)-N,N- dimethylpicolinamide | 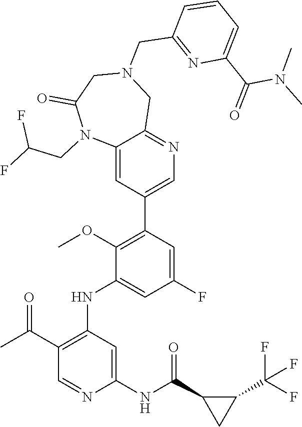 [SMILES: COc1c(Nc2cc(NC(=O)[C@@H]3C[C@H]3C(F)(F)F)ncc2C(C)=O)cc(F)cc1-c1cnc2c(c1)N(CC(F)F)C(=O)CN(Cc1cccc(C(=O)N(C)C)n1)C2] | LC-MS calc. for C₃₈H₃₇F₆N₈O₅ [M + H]+: m/z = 799.3; Found: 799.3. |
| 246 | (S)-6-((8-(3-((5-acetyl-2- (2,2-difluorocyclopropane-1- carboxamido)pyridin-4- yl)amino)-5-fluoro-2- methoxyphenyl)-1-(2,2- difluoroethyl)-2-oxo-1,2,3,5- tetrahydro-4H-pyrido[3,2- e][1,4]diazepin-4- yl)methyl)-N,N- dimethylpicolinamide | 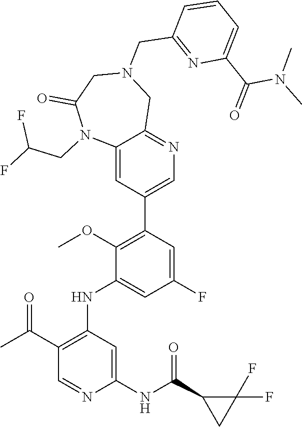 [SMILES: COc1c(Nc2cc(NC(=O)[C@@H]3CC3(F)F)ncc2C(C)=O)cc(F)cc1-c1cnc2c(c1)N(CC(F)F)C(=O)CN(Cc1cccc(C(=O)N(C)C)n1)C2] | LC-MS calc. for C₃₇H₃₆F₅N₈O₅ [M + H]⁺: m/z = 767.3; Found: 767.3. |
| 247 | (R)-6-((8-(3-((5-acetyl-2- (2,2-difluorocyclopropane- 1-carboxamido)pyridin-4- yl)amino)-2- methoxyphenyl)-1-ethyl-2- oxo-1,2,3,5-tetrahydro-4H- benzo[e][1,4]diazepin-4- yl)methyl)-N,N- dimethylpicolinamide | 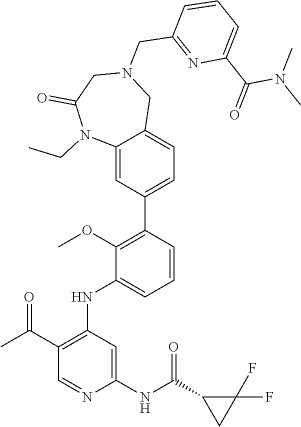 [SMILES: CCN1C(=O)CN(Cc2cccc(C(=O)N(C)C)n2)Cc2ccc(-c3cccc(Nc4cc(NC(=O)[C@H]5CC5(F)F)ncc4C(C)=O)c3OC)cc21] | LC-MS calc. for C₃₈H₄₀F₂N₇O₅ [M + H]⁺: m/z = 712.3; Found: 712.1. |

### Example 248. 6-((8-(3-((5-acetyl-2-(2,2-dimethylcyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide

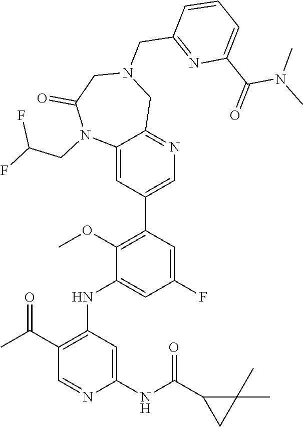 [SMILES: COc1c(Nc2cc(NC(=O)C3CC3(C)C)ncc2C(C)=O)cc(F)cc1-c1cnc2c(c1)N(CC(F)F)C(=O)CN(Cc1cccc(C(=O)N(C)C)n1)C2]

To a stirring solution of 6-((8-(3-((5-acetyl-2-aminopyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide (10.8 mg, 0.016 mmol) in pyridine (0.5 mL) at 0° C. was added 2,2-dimethylcyclopropane-1-carbonyl chloride (2.3 μL, 0.19 mmol). The reaction was stirred on ice for 1 h before being quenched with a mixture of MeOH, water, and TFA, diluted in MeOH, and purified by prep-LCMS (CSH-C18, 23.1-43.1% MeCN/0.2% TFA (aq) over 5 min) to afford the title compound (3.5 mg, 20% yield) as a TFA salt. LC-MS calc. for C₃₉H₄₂F₃N₈O₅[M+H]⁺: m/z=759.3; Found: 759.3.

### Example 249. 6-((5-((1-((3′,6′-dihydroxy-3-oxo-3H-spiro[isobenzofuran-1,9′-xanthen]-5-yl)amino)-1-thioxo-5,8,11-trioxa-2-azatridecan-13-yl)carbamoyl)pyridin-2-yl)amino)-4-((3-(1-(1-((6-(dimethylcarbamoyl)pyridin-2-yl)methyl)azetidin-3-yl)-1H-pyrazol-4-yl)-2-methoxyphenyl)amino)-N-methylpyridazine-3-carboxamide

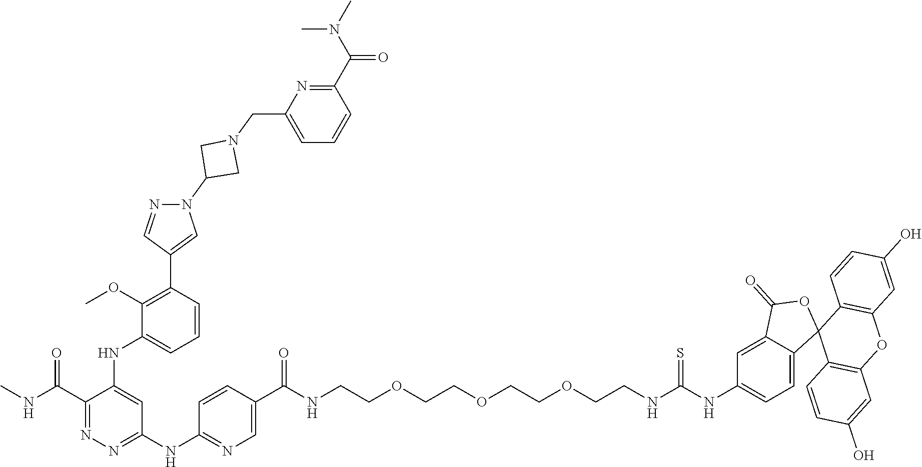 [SMILES: CNC(=O)c1nnc(Nc2ccc(C(=O)NCCOCCOCCOCCNC(=S)Nc3ccc4c(c3)C(=O)OC43c4ccc(O)cc4Oc4cc(O)ccc43)cn2)cc1Nc1cccc(-c2cnn(C3CN(Cc4cccc(C(=O)N(C)C)n4)C3)c2)c1OC]

### Example A. Biological Data

### JAK2V617F JH2 and JAK2 JH1 Binding Assay

Compounds were evaluated in a TR-FRET binding assay using recombinant JAK2 protein and a fluorescently labeled tracer. Recombinant JAK2 protein was purchased from BPS Bioscience or CARNA bio (JAK2 JH1, Carna P/N 08-445-20N; JAK2V617F JH2, BPS Bioscience 79498). Fluorescently labeled tracers were made in house (Example 249) or purchased from MedChem Express (JH2 Tracer, #HY-102055). TR-FRET assays were conducted in black 384-well low volume microplate (ProxiPlate, Revvity P/N 6008260) in 12 μL binding buffer (50 mM TRIS, pH 7.4, 10 mM MgCl₂, 0.01% Brij, 1 mM EGTA, 5 mM DTT, >133 nM Streptavidin-Tb cryptate (Revvity P/N 610SATLF) and 0.1% BSA). JAK2 protein at a final concentration of 0.1 nM (JAK2 JH1) or 0.5 nM (JAK2V617F JH2) was tested with 50 nM of tracer (Example 249 or JH2 Tracer). Compounds dissolved in DMSO were plated in a 9-point dilution scheme using an acoustic liquid handler (Echo 650T). The assay was incubated for 60 minutes at room temperature before reading by a microplate plate reader (BMG Labtech PHERAstar or Revvity Envision 2105).

The results are summarized below in Table 23.

TABLE 23
Binding IC₅₀ Values

| | JAK2V617F | Jak2 JH1 |
|---|---|---|
| Example | JH2 IC₅₀ (nM) | IC₅₀ (nM) |
| 1 | A | D |
| 2 | A | D |
| 3 | B | D |
| 4 | A | D |
| 5 | A | D |
| 6 | A | D |
| 7 | A | A |
| 8 | B | D |
| 9 | B | ND |
| 10 | A | D |
| 11 | A | D |
| 12 | A | D |
| 13 | A | D |
| 14 | A | D |
| 15 | A | D |
| 16 | A | ND |
| 17 | A | ND |
| 18 | A | ND |
| 19 | A | ND |
| 20 | A | ND |
| 21 | A | ND |
| 22 | A | ND |
| 23 | A | ND |
| 24 | A | ND |
| 25 | A | ND |
| 26 | A | ND |
| 27 | A | ND |
| 28 | A | ND |
| 29 | A | ND |
| 30 | A | ND |
| 31 | A | ND |
| 32 | A | ND |
| 33 | A | ND |
| 34 | A | ND |
| 35 | A | ND |
| 36 | A | D |
| 37 | A | ND |
| 38 | A | ND |
| 39 | A | ND |
| 40 | A | ND |
| 41 | A | ND |
| 42 | A | ND |
| 43 | A | ND |
| 44 | A | ND |
| 45 | A | ND |
| 46 | A | ND |
| 47 | A | ND |
| 48 | A | ND |
| 49 | A | ND |
| 50 | A | ND |
| 51 | A | ND |
| 52 | A | ND |
| 53 | A | ND |
| 54 | B | ND |
| 55 | A | ND |
| 56 | A | ND |
| 57 | A | ND |
| 58 | A | ND |
| 59 | A | ND |
| 60 | A | ND |
| 61 | A | ND |
| 62 | A | D |
| 63 | A | ND |
| 64 | A | ND |
| 65 | A | ND |
| 66 | A | ND |
| 67 | A | ND |
| 68 | A | ND |
| 69 | A | ND |
| 70 | A | ND |
| 71 | A | ND |
| 72 | A | ND |
| 73 | A | ND |
| 74 | A | ND |
| 75 | A | ND |
| 76 | A | ND |
| 77 | A | ND |
| 78 | A | ND |
| 79 | A | ND |
| 80 | A | ND |
| 81 | A | ND |
| 82 | A | ND |
| 83 | B | ND |
| 84 | A | ND |
| 85 | A | ND |
| 86 | A | ND |
| 87 | A | ND |
| 88 | A | ND |
| 89 | A | ND |
| 90 | A | ND |
| 91 | A | ND |
| 92 | A | ND |
| 93 | B | ND |
| 94 | B | ND |
| 95 | A | ND |
| 96 | A | ND |
| 97 | A | ND |
| 98 | A | ND |
| 99 | A | ND |
| 100 | A | ND |
| 101 | A | ND |
| 102 | A | ND |
| 103 | A | ND |
| 104 | A | ND |
| 105 | A | ND |
| 106 | A | ND |
| 107 | A | ND |
| 108 | A | ND |
| 109 | A | ND |
| 110 | A | ND |
| 111 | A | ND |
| 112 | A | ND |
| 113 | B | ND |
| 114 | A | ND |
| 115 | A | ND |
| 116 | A | ND |
| 117 | A | ND |
| 118 | A | D |
| 119 | A | ND |
| 120 | A | ND |
| 121 | A | ND |
| 122 | A | ND |
| 123 | A | D |
| 124 | A | ND |
| 125 | A | D |
| 126 | A | ND |
| 127 | A | ND |
| 128 | A | ND |
| 129 | A | ND |
| 130 | B | ND |
| 131 | A | ND |
| 132 | A | C |
| 133 | A | ND |
| 134 | A | ND |
| 135 | A | ND |
| 136 | A | ND |
| 137 | B | ND |
| 138 | A | ND |
| 139 | A | ND |
| 140 | B | ND |
| 141 | B | ND |
| 142 | A | ND |
| 143 | A | ND |
| 144 | A | ND |
| 145 | A | ND |
| 146 | B | ND |
| 147 | B | ND |
| 148 | B | ND |
| 149 | A | ND |
| 150 | A | ND |
| 151 | B | ND |
| 152 | B | ND |
| 153 | A | ND |
| 154 | A | ND |
| 155 | A | ND |
| 156 | A | ND |
| 157 | A | ND |
| 158 | A | ND |
| 159 | A | ND |
| 160 | A | ND |
| 161 | A | ND |
| 162 | A | ND |
| 163 | A | ND |
| 164 | A | ND |
| 165 | A | ND |
| 166 | B | ND |
| 167 | C | ND |
| 168 | B | ND |
| 169 | A | ND |
| 170 | A | ND |
| 171 | A | ND |
| 172 | A | ND |
| 173 | A | ND |
| 174 | A | ND |
| 175 | A | ND |
| 176 | A | ND |
| 177 | A | ND |
| 178 | A | ND |
| 179 | A | ND |
| 180 | A | ND |
| 181 | A | D |
| 182 | B | ND |
| 183 | A | ND |
| 184 | A | D |
| 185 | A | ND |
| 186 | B | ND |
| 187 | A | ND |
| 188 | A | ND |
| 189 | A | ND |
| 190 | A | ND |
| 191 | A | ND |
| 192 | A | ND |
| 193 | A | ND |
| 194 | A | ND |
| 195 | A | ND |
| 196 | A | ND |
| 197 | B | ND |
| 198 | B | ND |
| 199 | A | ND |
| 200 | A | ND |
| 201 | A | ND |
| 202 | A | ND |
| 203 | A | ND |
| 204 | A | ND |
| 205 | A | ND |
| 206 | A | ND |
| 207 | A | ND |
| 208 | A | ND |
| 209 | A | ND |
| 210 | A | ND |
| 211 | A | ND |
| 212 | A | ND |
| 213 | A | ND |
| 214 | A | ND |
| 215 | A | ND |
| 216 | A | ND |
| 217 | A | ND |
| 218 | A | ND |
| 219 | A | ND |
| 220 | A | ND |
| 221 | B | ND |
| 222 | A | ND |
| 223 | A | ND |
| 224 | A | ND |
| 225 | A | ND |
| 226 | A | ND |
| 227 | A | ND |
| 228 | A | ND |
| 229 | A | ND |
| 230 | A | ND |
| 231 | A | ND |
| 232 | A | ND |
| 233 | A | ND |
| 234 | A | ND |
| 235 | A | ND |
| 236 | C | ND |
| 237 | B | ND |
| 238 | A | ND |
| 239 | B | ND |
| 240 | B | ND |
| 241 | B | ND |
| 242 | B | ND |
| 243 | B | ND |
| 244 | A | ND |
| 245 | A | ND |
| 246 | A | ND |
| 247 | A | ND |
| 248 | A | ND |

In Table 23, a “A” denotes an IC₅₀ value of ≤50 nM; a “B” denotes an IC₅₀ value of 50 nM≤IC₅₀≤500 nM; a “C” denotes an IC₅₀ value of 500 nM≤IC₅₀≤5000 nM; a “D” denotes an IC₅₀ value of >5000 nM; ND=not determined.

pSTAT5 Assay

The inhibitory activity of compounds was evaluated by measuring phospho-STAT5 (phosphorylated on Tyr694/699) as a readout of the JAK2-STAT5 pathway using AlphaLISA (Revvity catalog number: ALSU-PST5). These studies were conducted in SET2 cell lines. SET2 cells were maintained in a 37° C. incubator at 5% CO₂ in the following media: RPMI 1640, ATCC© Modification (Gibco, A10491-01) supplemented with 20% v/v heat inactivated FBS (Gibco, 26140-079), 1% penicillin streptomycin (Gibco, 15140-122). Compounds dissolved in DMSO were plated in a 9-point dilution scheme into 384-well plates using an acoustic liquid handler (Echo 650T). Cells were seeded on top of dosed compounds at a density of 6,000 cells/well in HBSS (ThermoScientific, catalogue number J67763.AP). After 2 hours of treatment, cells were lysed for thirty minutes and incubated with detection reagents per AlphaLISA kit material and manufacturer's instructions. Signal was measured with plate reader (Envision 2105, Perkin Elmer).

The results are summarized below in Table 24.

TABLE 24
SET2 pSTAT5 IC₅₀ Values

| Example | SET2 pSTAT5 IC₅₀ (nM) |
|---|---|
| 1 | B |
| 2 | A |
| 3 | B |
| 4 | A |
| 5 | B |
| 6 | A |
| 7 | A |
| 8 | B |
| 9 | B |
| 10 | B |
| 11 | C |
| 12 | B |
| 13 | B |
| 14 | A |
| 15 | B |
| 16 | A |
| 17 | A |
| 18 | A |
| 19 | A |
| 20 | B |
| 21 | A |
| 22 | A |
| 23 | A |
| 24 | B |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | A |
| 43 | A |
| 44 | A |
| 45 | A |
| 46 | A |
| 47 | B |
| 48 | A |
| 49 | A |
| 50 | A |
| 51 | A |
| 52 | A |
| 53 | A |
| 54 | B |
| 55 | A |
| 56 | A |
| 57 | A |
| 58 | A |
| 59 | A |
| 60 | A |
| 61 | A |
| 62 | B |
| 63 | A |
| 64 | B |
| 65 | A |
| 66 | A |
| 67 | A |
| 68 | A |
| 69 | A |
| 70 | A |
| 71 | A |
| 72 | A |
| 73 | A |
| 74 | A |
| 75 | A |
| 76 | A |
| 77 | A |
| 78 | A |
| 79 | A |
| 80 | A |
| 81 | A |
| 82 | A |
| 83 | A |
| 84 | A |
| 85 | A |
| 86 | A |
| 87 | A |
| 88 | A |
| 89 | A |
| 90 | B |
| 91 | A |
| 92 | A |
| 93 | B |
| 94 | B |
| 95 | A |
| 96 | A |
| 97 | A |
| 98 | A |
| 99 | A |
| 100 | A |
| 101 | B |
| 102 | A |
| 103 | A |
| 104 | A |
| 105 | A |
| 106 | A |
| 107 | A |
| 108 | A |
| 109 | A |
| 110 | A |
| 111 | A |
| 112 | A |
| 113 | C |
| 114 | A |
| 115 | B |
| 116 | A |
| 117 | A |
| 118 | A |
| 119 | A |
| 120 | A |
| 121 | A |
| 122 | A |
| 123 | A |
| 124 | A |
| 125 | A |
| 126 | B |
| 127 | B |
| 128 | B |
| 129 | B |
| 130 | B |
| 131 | A |
| 132 | A |
| 133 | A |
| 134 | B |
| 135 | A |
| 136 | B |
| 137 | B |
| 138 | A |
| 139 | A |
| 140 | B |
| 141 | B |
| 142 | A |
| 143 | A |
| 144 | B |
| 145 | B |
| 146 | B |
| 147 | B |
| 148 | B |
| 149 | A |
| 150 | A |
| 151 | B |
| 152 | B |
| 153 | B |
| 154 | A |
| 155 | A |
| 156 | A |
| 157 | A |
| 158 | A |
| 159 | A |
| 160 | A |
| 161 | A |
| 162 | A |
| 163 | B |
| 164 | B |
| 165 | B |
| 166 | B |
| 167 | B |
| 168 | B |
| 169 | A |
| 170 | B |
| 171 | D |
| 172 | A |
| 173 | A |
| 174 | B |
| 175 | A |
| 176 | A |
| 177 | A |
| 178 | A |
| 179 | A |
| 180 | B |
| 181 | A |
| 182 | B |
| 183 | B |
| 184 | A |
| 185 | B |
| 186 | B |
| 187 | A |
| 188 | A |
| 189 | B |
| 190 | A |
| 191 | B |
| 192 | B |
| 193 | A |
| 194 | A |
| 195 | A |
| 196 | A |
| 197 | C |
| 198 | C |
| 199 | B |
| 200 | A |
| 201 | A |
| 202 | B |
| 203 | A |
| 204 | A |
| 205 | B |
| 206 | B |
| 207 | A |
| 208 | A |
| 209 | A |
| 210 | A |
| 211 | A |
| 212 | B |
| 213 | A |
| 214 | A |
| 215 | A |
| 216 | A |
| 217 | A |
| 218 | A |
| 219 | A |
| 220 | A |
| 221 | B |
| 222 | A |
| 223 | B |
| 224 | A |
| 225 | A |
| 226 | A |
| 227 | A |
| 228 | B |
| 229 | B |
| 230 | A |
| 231 | A |
| 232 | A |
| 233 | A |
| 234 | A |
| 235 | A |
| 236 | C |
| 237 | B |
| 238 | B |
| 239 | B |
| 240 | B |
| 241 | B |
| 242 | B |
| 243 | B |
| 244 | B |
| 245 | A |
| 246 | B |
| 247 | A |
| 248 | B |

In Table 24, a “A” denotes an IC₅₀ value of ≤200 nM; a “B” denotes an IC₅₀ value of 200 nM<IC₅₀≤1000 nM; a “C” denotes an IC₅₀ value of >1000 nM. ND=not determined.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A compound of Formula (I) 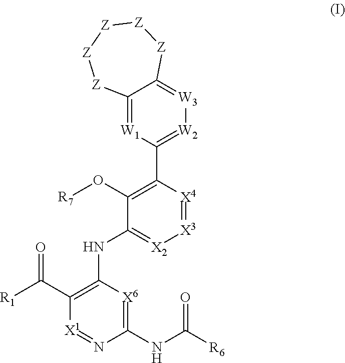
or a pharmaceutically acceptable salt or solvate or N-oxide thereof, wherein
each of W₁, W₂, and W₃ is independently N or CR₈;
each Z is independently O, S, SO₂, NR₄, NR₉ or C(R₈)₂, wherein at least 3 Z are C(R₈)₂ and wherein at least one Z is NR₄;
each R₈ is independently H, D, halogen, —OH, —CN, —NO₂, —C₁-C₆alkyl, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
or any two R₈ groups attached to the same carbon form ═O;
or any two R₈ groups attached to the same carbon form a 3-6 membered spirocycloalkyl or spiroheterocycloalkyl ring;
or any two R₈ groups attached to adjacent carbon atoms form a 3-6 membered cycloalkyl or heterocycloalkyl ring;
each R₄ is independently -L¹-L²-L³;
each R₉ is independently H, —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl, —C(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b}; wherein each of —C₁-C₆alkyl, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl, spirocycloalkenyl, spiroheterocycloalkenyl can be optionally substituted by 1-6 R_{f} groups;
each L¹ is independently absent, or is independently —(CR^{c}R^{d})ₚ—, —(CR^{a}R^{c})ₚO—, —S(O)—, —S(O)₂—, —C(═O)—, —C(═O)O—, —C(═O)NR^{a}—, —S(═O)NR^{a}—, —S(═O)₂NR^{a}—, or aryl, heteroaryl, cycloalkyl, cycloalkenyl, heterocycloalkyl, or heterocycloalkenyl;
each L² is independently absent, or is independently aryl, heteroaryl, cycloalkyl, cycloalkenyl, heterocycloalkyl, or heterocycloalkenyl;
each L³ is independently absent, or is independently H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
p is 1, 2, 3 or 4;
each X₁, X₂, X₃, X₄, and X₆ is independently N or CR₁₀;
each R₁₀ is independently H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
R₁ is H, D, —C₁-C₆alkyl, —C₁-C₆haloalkyl, C₁-C₈ alkoxide, —C₂-C₆alkenyl, —C₂-C₆alkynyl, —NHR₅;
R₅ is H, —C₁-C₆alkyl, C₁-C₈ alkoxide, —C₂-C₆alkenyl, —C₂-C₆alkynyl, cycloalkyl, cycloalkenyl, heterocycloalkyl, or heterocycloalkenyl;
R₆ is H, D, —OH, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl or spiroheterocycloalkenyl;
R₇ is H, —C₁-C₆alkyl, —C₁-C₆haloalkyl, —C₂-C₆alkenyl, or cycloalkyl;
each R^{a} is independently H, —C₁-C₁₀alkyl, —C₂-C₁₀ alkenyl, —C₂-C₁₀ alkynyl, aryl, cycloalkyl, cycloalkenyl, heteroaryl, heterocycloalkyl, or heterocycloalkenyl;
each R^{b}, is independently H, D, —C₁-C₆ alkyl, —C₂-C₆ alkenyl, —C₂-C₆ alkynyl, aryl, cycloalkyl, cycloalkenyl, heteroaryl, heterocycloalkyl, or heterocycloalkenyl;
each R^{c} or R^{d} is independently H, D, —C₁-C₁₀ alkyl, —C₂-C₆ alkenyl, —C₂-C₆ alkynyl, —OC₁-C₆alkyl, —O-cycloalkyl, aryl, heteroaryl, cycloalkyl, cycloalkenyl, heterocycloalkyl, or heterocycloalkenyl;
or R^{c} and R^{d}, together with the atom to which they are both attached, form a monocyclic or multicyclic heterocycloalkyl, or a monocyclic or multicyclic heterocyclo-alkenyl group;
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, heterocycloalkenyl, spirocycloalkyl, spiroheterocycloalkyl and spiroheterocycloalkenyl groups in any of R₁-R₁₀ and L¹-L³ is optionally substituted by 1-6 R^{f} groups; and
each R^{f} is independently H, D, oxo, halogen, C₁-C₈ alkoxy, C₁-C₈ alkyl, C₁₋₆ haloalkyl, C₁₋₆ haloalkoxy, —OH, —CN, —NO₂, —C₂₋₆ alkenyl, —C₂₋₆ alkynyl, C₆₋₁₀ aryl, C₅₋₁₂ heteroaryl, C₃₋₈ cycloalkyl, C₃₋₈ cycloalkenyl, C₃₋₈ heterocycloalkyl, C₃₋₈ heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)R^{c}R^{d}, —P(O)(OR^{b})(OR^{b}), —B(OR^{c})(OR^{d}), —S(O)₂R^{b}, —C(O)NR^{b}OR^{b}, —S(O)₂OR^{b}, —OS(O)₂OR^{b}, or —OPO(OR^{b})(OR^{b}); wherein said C₁-C₈ alkyl is optionally substituted by 1-6 groups selected from D, halogen, —OH, —CN, —OR^{a}, —SR^{a}, —NR^{a}R^{d} or NR^{c}R^{d}.

2-7. (canceled)

8. The compound of claim 1, wherein the compound is a compound of Formula (II): 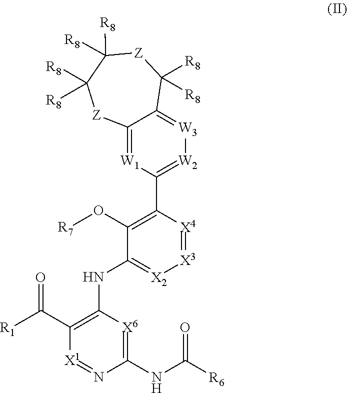
or a pharmaceutically acceptable salt or solvate or N-oxide thereof.

9. The compound of claim 8, wherein the compound is a compound of Formula (III), Formula (IV) or Formula (V): 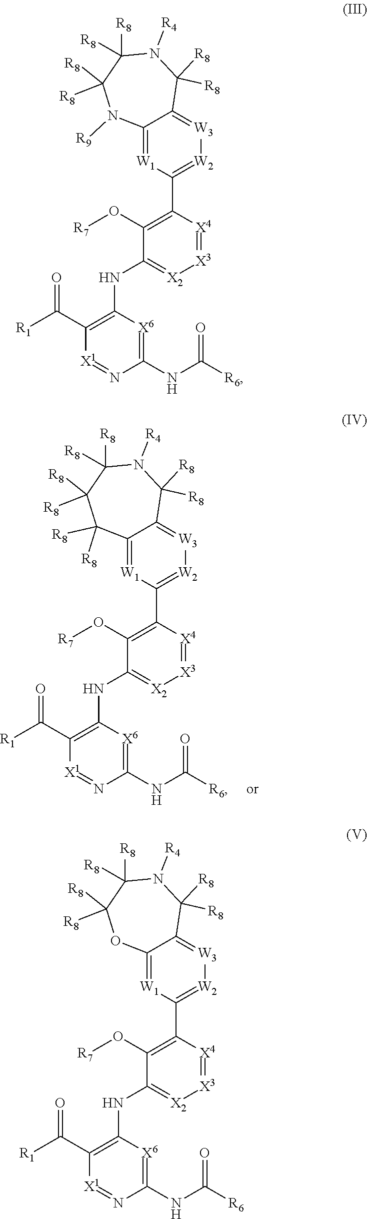
or a pharmaceutically acceptable salt or solvate or N-oxide thereof.

10. The compound of claim 8, wherein two R₈ groups attached to the same carbon join together to form ═O.

11. The compound of claim 1, wherein the compound is a compound of Formula (VI), Formula (VII), Formula (VIII), Formula (XIX), Formula (X), Formula (XI), Formula (XII), Formula (XIII), Formula (XIV), or Formula (XV): 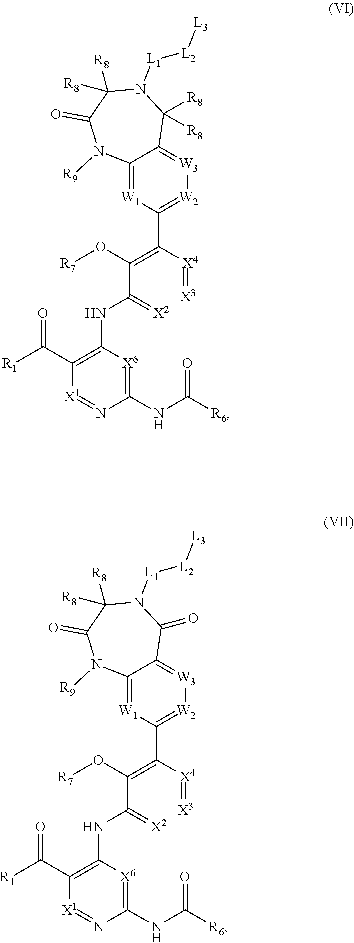 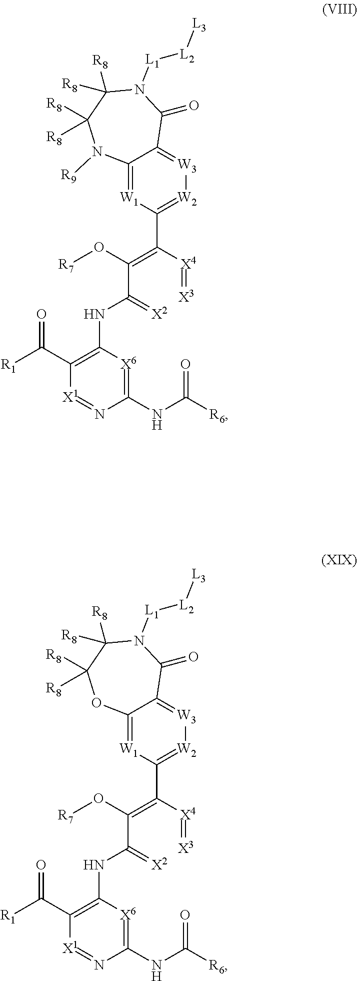 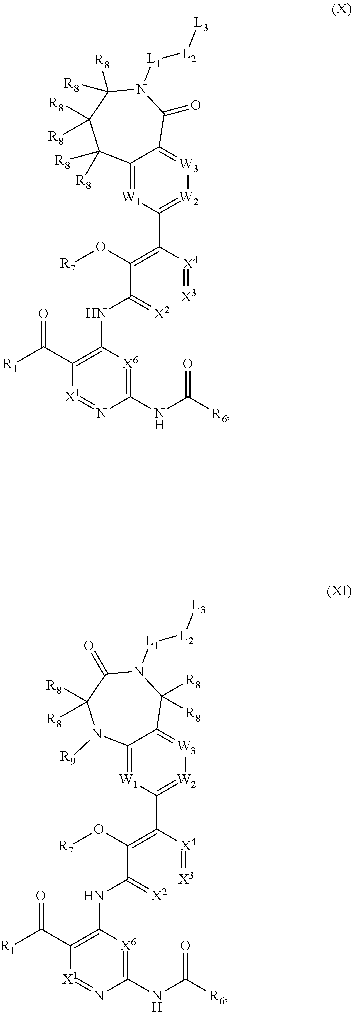 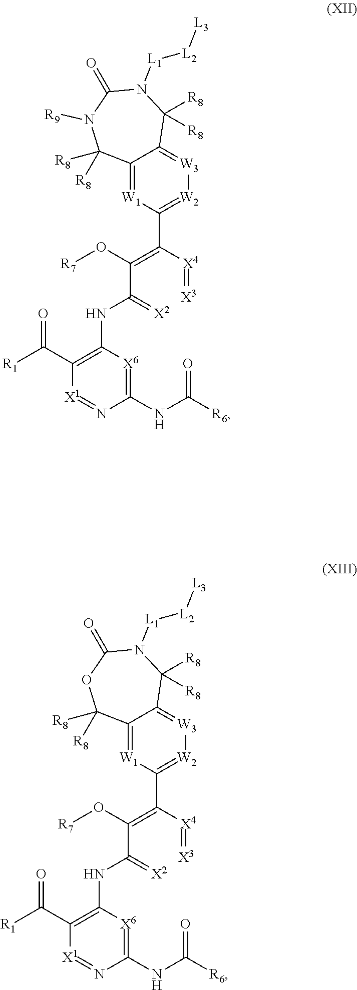 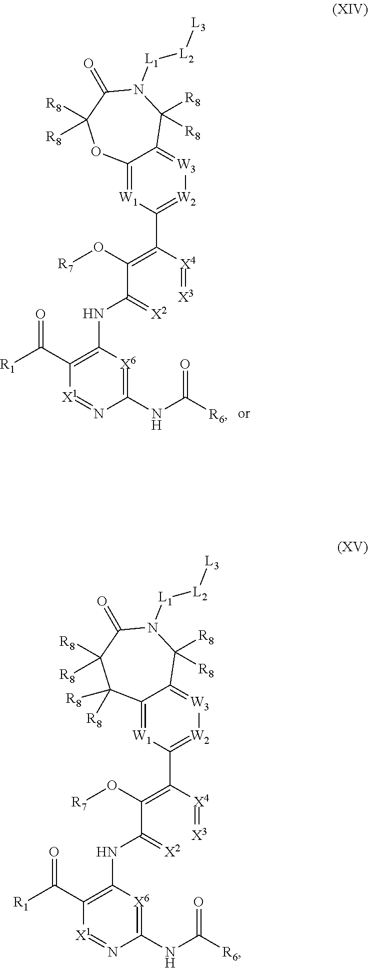
or a pharmaceutically acceptable salt or solvate or N-oxide thereof; wherein
L¹ is absent, —(CR^{c}R^{d})ₚ—, —S(O)—, —S(O)₂—, or —C(═O)—;
L² is absent, aryl or heteroaryl; and
L³ is H, D, halogen, —OH, —CN, —NO₂, —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, or heterocycloalkenyl, —OR^{a}, —SR^{a}, —NR^{c}R^{d}, —NR^{a}R^{c}, —C(O)R^{b}, —OC(O)R^{b}, —C(O)OR^{b}, —C(O)NR^{c}R^{d}, —S(O)R^{b}, —S(O)₂NR^{c}R^{d}, —S(O)(═NR^{b})R^{b}, —SF₅, —P(O)R^{b}R^{b}, —P(O)(OR^{b})(OR^{b}), —B(OR^{c})(OR^{d}) or —S(O)₂R^{b};
wherein any of the —C₁-C₆alkyl, C₁-C₈ alkoxide, —C₂-C₆alkenyl, —C₂-C₆alkynyl, aryl, heteroaryl, cycloalkyl, cycloalkenyl, heterocycloalkyl, and heterocycloalkenyl groups of L² and L³ are optionally substituted by 1-6 R_{f} groups.

12. The compound of claim 1, wherein each of X₂, X₃, and X₄ is CH.

13. The compound of claim 1, wherein at least one of X₂, X₃ and X₄ is N.

14-21. (canceled)

22. The compound of claim 21, wherein R₆ is cycloalkyl optionally substituted by 1-6 R_{f} groups.

23. The compound of claim 22, wherein R₆ is cyclopropyl optionally substituted by 1-6 R_{f} groups.

24. The compound of claim 22, wherein R₆ is fluorine substituted cyclopropane.

25. The compound of claim 11, wherein L¹ is —(CR^{c}R^{d})ₚ—.

26. (canceled)

27. The compound of claim 11, wherein L² is phenyl or pyridyl.

28. The compound of claim 11, wherein L³ is —C(O)NR^{c}R^{d} wherein R^{c} and R^{d} are each independently —C₁-C₁₀ alkyl;
or R^{c} and R^{d}, together with the atom to which they are both attached, form a monocyclic or multicyclic heterocycloalkyl.

29. (canceled)

30. The compound of claim 1, wherein R₁ is —NHR₅.

31. The compound of claim 30, wherein R₅ is H, —C₁-C₆alkyl, or C₁-C₈ alkoxide, wherein the —C₁-C₆alkyl and C₁-C₈ alkoxide groups are optionally substituted by 1-6 R_{f} groups.

32. The compound of claim 31, wherein R₅ is —C₁-C₆alkyl optionally substituted by 1-6 R_{f} groups.

33-35. (canceled)

36. The compound of claim 1, wherein R₁ is C₁-C₆ alkyl optionally substituted by 1-6 R group selected from H, D or halogen or —C₁-C₆haloalkyl optionally substituted by 1-6 R groups selected from H, D or halogen.

37-38. (canceled)

39. The compound of claim 1, wherein R₇ is —C₁-C₆alkyl.

40. (canceled)

41. The compound of claim 1 that is:
6-(Cyclopropanecarboxamido)-4-((3-(1-ethyl-4-((6-(dimethylcarbamoyl)pyridin-2-yl)methyl)-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-isopropyl-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-(3-(dimethylcarbamoyl)-2-fluorobenzyl)-1-ethyl-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(1-cyclopropyl-4-((6-(dimethylcarbamoyl)pyridin-2-yl)methyl)-2-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(2-((6-(dimethylcarbamoyl)pyridin-2-yl)methyl)-1-oxo-2,3,4,5-tetrahydro-1H-benzo[c]azepin-7-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-5-oxo-2,3,4,5-tetrahydrobenzo[f][1,4]oxazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-6-fluoro-5-oxo-2,3,4,5-tetrahydrobenzo[f][1,4]oxazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-methyl-5-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-ethyl-5-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-isopropyl-5-oxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-methyl-2,5-dioxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-ethyl-2,5-dioxo-2,3,4,5-tetrahydro-1H-benzo[e][1,4]diazepin-8-yl)-2-methoxyphenyl)amino)-N-methylpyridazine-3-carboxamide;
6-(Cyclopropanecarboxamido)-4-((3-(4-((6-(dimethylcarbamoyl)pyridin-2-yl)methyl)-1-isopropyl-2,5-dioxo-2,3,4,5-tetrahydro-1H-benzo[e][1,4]diazepin-8-yl)-2-methoxyphenyl)amino)-N-methylpyridazine-3-carboxamide;
6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
3-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-2-fluoro-N,N-dimethylbenzamide;
6-((8-(3-((5-Acetyl-2-(cyclopropanecarboxamido)pyridin-4-yl)amino)-2-methoxyphenyl)-1-(2-fluoroethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(2,2,2-trifluoroethyl)-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(cyanomethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(oxetan-3-yl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(3-((5-Acetyl-2-((1R,2S)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
(S)-6-((8-(3-((5-Acetyl-2-(spiro[2.2]pentane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
(R)-6-((8-(3-((5-Acetyl-2-(spiro[2.2]pentane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-((1S,2R)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-((1R,2R)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-((1S,2S)-2-fluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-cyclopropyl-2-oxo-1,2,3,5-tetrahydro-4H-benzo[e][1,4]diazepin-4-yl)methyl)-N,N-dimethylpicolinamide;
or a pharmaceutically acceptable salt thereof.

42. The compound of claim 1 that is:
6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(S)-6-((8-(3-((5-acetyl-2-(cyclopropanecarboxamido)pyridin-4-yl)amino)-5-fluoro-2-methoxyphenyl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-(2-fluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-isopropyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-isobutyl-2-oxo-1,2,3,5-tetrahydro-4H-pyrido[3,2-e][1,4]diazepin-4-yl)methyl)-N,N-dimethylpicolinamide;
or a pharmaceutically acceptable salt thereof.

43. The compound of claim 1 that is:
6-((8-(3-((5-acetyl-2-(cyclopropanecarboxamido)pyridin-4-yl)amino)-2-methoxyphenyl)-1-cyclobutyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(1-fluoropropan-2-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(thiazol-5-ylmethyl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(cyclopropylmethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-cyclopentyl-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-2-oxo-1-((S)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-2-oxo-1-((S)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-(2-fluoroethyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2R)-2-methylcyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1S,2S)-2-methylcyclopropane-1-carboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-(2-fluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-cyclobutyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-6-fluoro-2-oxo-1-((S)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-2-oxo-1-((R)-tetrahydrofuran-3-yl)-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-(2,2-difluoroethyl)-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-(2-fluoroethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-6-fluoro-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxy-5-(trifluoromethyl)phenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxy-5-(trifluoromethyl)phenyl)-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-(2-fluoro-2-methylpropyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-(2-methoxy-2-methylpropyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-isobutyl-2-oxo-1,2,3,5-tetrahydro-4H-pyrido[3,2-e][1,4]diazepin-4-yl)methyl)-N,N-dimethylpicolinamide;
3-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylbenzamide;
6-((8-(3-((5-Acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methyl-N-(2,2,2-trifluoroethyl)picolinamide;
N-(5-acetyl-4-((3-(1-(2,2-difluoroethyl)-4-((6-(3-hydroxypyrrolidine-1-carbonyl)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-2-oxo-4-((6-(pyrrolidine-1-carbonyl)pyridin-2-yl)methyl)-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-(2,2-difluoroethyl)-N-methylpicolinamide;
6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-(2-hydroxyethyl)-N-methylpicolinamide;
6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-(tert-butyl)-N-methylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-methyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-2-methoxyphenyl)-6-fluoro-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-6-fluoro-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-6-fluoro-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-5-fluoro-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxy-5-methylphenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxy-5-(trifluoromethyl)phenyl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-4-fluoro-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(R)-6-((8-(3-((5-acetyl-2-(cyclopropanecarboxamido)pyridin-4-yl)amino)-2-methoxyphenyl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
(S)-6-((8-(3-((5-acetyl-2-(cyclopropanecarboxamido)pyridin-4-yl)amino)-2-methoxyphenyl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-isopropyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-ethyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-isopropyl-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-((1s,3s)-3-cyanocyclobutyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(1-cyanoethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxy-5-(trifluoromethyl)phenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-ethyl-7-fluoro-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2-cyanoethyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-methyl-2-oxo-1,2,3,5-tetrahydro-4H-pyrido[4,3-e][1,4]diazepin-4-yl)methyl)-N,N-dimethylpicolinamide;
6-((8-(3-((5-acetyl-2-((1S,2R)-2-fluorocyclopropane-1-carboxamido)pyridin-4-yl)amino)-2-methoxyphenyl)-1-(2-methoxyethyl)-2-oxo-1,2,3,5-tetrahydro-4H-pyrido[4,3-e][1,4]diazepin-4-yl)methyl)-N,N-dimethylpicolinamide;
6-((8-(3-((5-acetyl-2-((1R,2S)-2-fluorocyclopropane-1-carboxamido)pyridin-4-yl)amino)-5-fluoro-2-methoxyphenyl)-1-methyl-2-oxo-1,2,3,5-tetrahydro-4H-pyrido[4,3-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-isopropyl-2-oxo-1,2,3,5-tetrahydro-4H-pyrido[4,3-e][1,4]diazepin-4-yl)methyl)-N,N-dimethylpicolinamide;
6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[4,3-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2,2-difluoropropyl)-2-oxo-1,2,3,5-tetrahydro-4H-pyrido[3,2-e][1,4]diazepin-4-yl)methyl)-N,N-dimethylpicolinamide;
(R)-6-((8-(3-((5-acetyl-2-(cyclopropanecarboxamido)pyridin-4-yl)amino)-2-methoxyphenyl)-1-(1-methoxypropan-2-yl)-2-oxo-1,2,3,5-tetrahydro-4H-pyrido[3,2-e][1,4]diazepin-4-yl)methyl)-N,N-dimethylpicolinamide;
(S)-6-((8-(3-((5-acetyl-2-(cyclopropanecarboxamido)pyridin-4-yl)amino)-2-methoxyphenyl)-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-6-fluoro-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(2,2-difluoroethyl)-6-fluoro-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-ethyl-6-fluoro-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-methyl-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-isopropyl-2-oxo-1,2,3,5-tetrahydro-4H-benzo[e][1,4]diazepin-4-yl)methyl)-N,N-dimethylpicolinamide;
6-((8-(2-((5-acetyl-2-(cyclopropanecarboxamido)pyridin-4-yl)amino)-3-methoxypyridin-4-yl)-1-isopropyl-2-oxo-1,2,3,5-tetrahydro-4H-benzo[e][1,4]diazepin-4-yl)methyl)-N,N-dimethylpicolinamide;
6-((8-(4-((5-acetyl-2-((1R,2S)-2-fluorocyclopropane-1-carboxamido)pyridin-4-yl)amino)-3-methoxypyridin-2-yl)-1-methyl-2-oxo-1,2,3,5-tetrahydro-4H-benzo[e][1,4]diazepin-4-yl)methyl)-N,N-dimethylpicolinamide;
6-((8-(3-((5-acetyl-2-((1S,2S)-2-methylcyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2R)-2-methylcyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-(8-(3-((5-acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-2-oxo-1-((S)-tetrahydrofuran-3-yl)-1,2,3,5-tetrahydro-4H-benzo[e][1,4]diazepin-4-yl)methyl)-N,N-dimethylpicolinamide;
6-((8-(2-((5-acetyl-2-(cyclopropanecarboxamido)pyridin-4-yl)amino)-3-methoxypyridin-4-yl)-1-isopropyl-2-oxo-1,2,3,5-tetrahydro-4H-pyrido[3,2-e][1,4]diazepin-4-yl)methyl)-N,N-dimethylpicolinamide;
6-((8-(2-((5-acetyl-2-(cyclopropanecarboxamido)pyridin-4-yl)amino)-3-methoxypyridin-4-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-neopentyl-2-oxo-1,2,3,5-tetrahydro-4H-pyrido[3,2-e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-cyclobutyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(2,2-difluoroethyl)-2-oxo-1,2,3,5-tetrahydro-4H-benzo[e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-2-oxo-1-(tetrahydrofuran-3-yl)-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2S)-2-methylcyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-methylcyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1R,2R)-2-methylcyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1R,2S)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2S)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1R,2R)-2-fluorocyclopropane-1-carboxamido)pyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((2-(cyclopropanecarboxamido)-5-propionylpyridin-4-yl)amino)-3-methoxypyridin-2-yl)-1-isopropyl-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-5-chloro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(R)-6-((8-(4-((5-acetyl-2-(cyclopropanecarboxamido)pyridin-4-yl)amino)-3-methoxypyridin-2-yl)-1-(1-methoxypropan-2-yl)-2-oxo-1,2,3,5-tetrahydro-4H-pyrido[3,2-e][1,4]diazepin-4-yl)methyl)-N,N-dimethylpicolinamide;
(1R,2S)—N-(5-acetyl-4-((5-fluoro-2-methoxy-3-(1-methyl-4-((6-(morpholine-4-carbonyl)pyridin-2-yl)methyl)-2-oxo-2,3,4,5-tetrahydro-1H-benzo[e][1,4]diazepin-8-yl)phenyl)amino)pyridin-2-yl)-2-fluorocyclopropane-1-carboxamide;
N-(5-acetyl-4-((3-(4-((6-(3,3-difluoropyrrolidine-1-carbonyl)pyridin-2-yl)methyl)-1-ethyl-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-((1-methyl-1H-pyrazol-4-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-((6-((3aR,6aR)-hexahydro-1H-furo[3,4-c]pyrrole-5-carbonyl)pyridin-2-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-((6-(morpholine-4-carbonyl)pyridin-2-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(4-((3-(4-([1,2,4]triazolo[1,5-a]pyridin-5-ylmethyl)-1-ethyl-2-oxo-2,3,4,5-tetrahydro-1H-benzo[e][1,4]diazepin-8-yl)-2-methoxyphenyl)amino)-5-acetylpyridin-2-yl)cyclopropanecarboxamide;
N-(4-((3-(4-([1,2,4]triazolo[1,5-a]pyridin-8-ylmethyl)-1-ethyl-2-oxo-2,3,4,5-tetrahydro-1H-benzo[e][1,4]diazepin-8-yl)-2-methoxyphenyl)amino)-5-acetylpyridin-2-yl)cyclopropanecarboxamide;
N-(5-acetyl-4-((3-(1-ethyl-4-(imidazo[1,5-a]pyridin-8-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-(imidazo[1,2-a]pyridin-8-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-2-oxo-4-(pyrazolo[1,5-a]pyridin-7-ylmethyl)-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-(imidazo[1,2-a]pyridin-5-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-((6-(methylsulfonyl)pyridin-2-yl)methyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-ethyl-4-(isoquinolin-1-ylmethyl)-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(4-((4-cyanopyridin-2-yl)methyl)-1-ethyl-2-oxo-2,3,4,5-tetrahydro-1H-benzo[e][1,4]diazepin-8-yl)-2-methoxyphenyl)amino)pyridin-2-yl)cyclopropanecarboxamide;
N-(5-acetyl-4-((3-(1-(2,2-difluoroethyl)-4-(imidazo[1,5-a]pyridin-8-ylmethyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-(imidazo[1,2-a]pyridin-8-ylmethyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-(3-(difluoromethyl)-2-fluorobenzyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(4-(3-chlorobenzyl)-1-(2,2-difluoroethyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-((1-methyl-2-oxo-1,2-dihydropyridin-3-yl)methyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-2-oxo-4-((6-oxo-1,6-dihydropyridin-2-yl)methyl)-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-4-((6-(difluoromethyl)pyridin-2-yl)methyl)-2-oxo-2,3,4,5-tetrahydro-1H-pyrido[3,2-e][1,4]diazepin-8-yl)-2-methoxyphenyl)amino)pyridin-2-yl)cyclopropanecarboxamide;
N-(5-acetyl-4-((3-(1-(2,2-difluoroethyl)-2-oxo-4-(pyridin-2-ylmethyl)-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-2-oxo-4-(pyridin-3-ylmethyl)-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-4-((6-(hydroxymethyl)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-2-oxo-4-(thiazol-4-ylmethyl)-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
6-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methoxy-N-methylpicolinamide;
3-((8-(3-((5-acetyl-2-(cyclopropanecarboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N-methoxy-N-methylbenzamide;
N-(5-acetyl-4-((3-(4-((6-aminopyridin-2-yl)methyl)-1-(2,2-difluoroethyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-4-((6-(3-methylureido)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-acetyl-4-((3-(1-(2,2-difluoroethyl)-4-((6-((N-methylsulfamoyl)amino)pyridin-2-yl)methyl)-2-oxo-2,3,4,5-tetrahydro-1H-pyrido[3,2-e][1,4]diazepin-8-yl)-5-fluoro-2-methoxyphenyl)amino)pyridin-2-yl)cyclopropanecarboxamide;
N-(5-Acetyl-4-((2-methoxy-3-(1-methyl-2-oxo-4-((6-(tetrahydrofuran-2-yl)pyridin-2-yl)methyl)-2,3,4,5-tetrahydro-1H-benzo[e][1,4]diazepin-8-yl)phenyl)amino)pyridin-2-yl)cyclopropanecarboxamide;
N-(5-Acetyl-4-((2-methoxy-3-(4-((6-(3-methoxyoxetan-3-yl)pyridin-2-yl)methyl)-1-methyl-2-oxo-2,3,4,5-tetrahydro-1H-benzo[e][1,4]diazepin-8-yl)phenyl)amino)pyridin-2-yl)cyclopropanecarboxamide;
N-(5-Acetyl-4-((3-(4-((6-acetylpyridin-2-yl)methyl)-1-methyl-2-oxo-2,3,4,5-tetrahydro-1H-benzo[e][1,4]diazepin-8-yl)-2-methoxyphenyl)amino)pyridin-2-yl)cyclopropanecarboxamide;
6-(2-(8-(3-((5-Acetyl-2-(cyclopropanecarboxamido)pyridin-4-yl)amino)-2-methoxyphenyl)-1-methyl-2-oxo-1,2,3,5-tetrahydro-4H-benzo[e][1,4]diazepin-4-yl)propan-2-yl)-N,N-dimethylpicolinamide;
6-((8-(3-((5-Acetyl-2-(cyclopropanecarboxamido)pyridin-4-yl)amino)-2-methoxyphenyl)-1-(tert-butyl)-2-oxo-1,2,3,5-tetrahydro-4H-pyrido[3,2-e][1,4]diazepin-4-yl)methyl)-N,N-dimethylpicolinamide;
(S)-6-((8-(3-((5-Acetyl-2-(cyclopropanecarboxamido)pyridin-4-yl)amino)-2-methoxyphenyl)-1-(1-acetylpyrrolidin-3-yl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(azetidin-3-yl)-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxy-6-(trifluoromethyl)pyridin-2-yl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxy-6-(trifluoromethyl)pyridin-2-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(4-((5-acetyl-2-((1S,2S)-2-methylcyclopropane-1-carboxamido)pyridin-4-yl)amino)-3-methoxy-6-(trifluoromethyl)pyridin-2-yl)-2-oxo-1-((S)-tetrahydrofuran-3-yl)-1,2,3,5-tetrahydro-4H-pyrido[3,2-e][1,4]diazepin-4-yl)methyl)-N,N-dimethylpicolinamide;
(S)-6-((8-(2-((5-Acetyl-2-(cyclopropanecarboxamido)pyridin-4-yl)amino)-3-methoxy-6-(trifluoromethyl)pyridin-4-yl)-2-oxo-1-(tetrahydrofuran-3-yl)-1,2,3,5-tetrahydro-4H-pyrido[3,2-e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-(bicyclo[1.1.1]pentan-1-yl)-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-(3-fluorobicyclo[1.1.1]pentan-1-yl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-(bicyclo[1.1.1]pentan-1-yl)-2-oxo-1,2,3,5-tetrahydro-4H-pyrido[3,2-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-(2-oxabicyclo[2.1.1]hexan-4-yl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-1-isobutyl-2-oxo-1,2,3,5-tetrahydro-4H-pyrido[3,2-e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-cyclopentyl-2-oxo-1,2,3,5-tetrahydro-4H-pyrido[3,2-e][1,4]diazepin-4-yl)methyl)-N,N-dimethylpicolinamide;
6-((8-(3-((5-Acetyl-2-((1R,2S)-2-fluorocyclopropane-1-carboxamido)pyridin-4-yl)amino)-2-methoxyphenyl)-2-oxo-1-((S)-tetrahydro-2H-pyran-3-yl)-1,2,3,5-tetrahydro-4H-pyrido[3,2-e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-1-(sec-butyl)-2-oxo-1,2,3,5-tetrahydro-4H-pyrido[3,2-e][1,4]diazepin-4-yl)methyl)-N,N-dimethylpicolinamide;
(R)-6-((8-(4-((5-acetyl-2-(cyclopropanecarboxamido)pyridin-4-yl)amino)-3-methoxypyridin-2-yl)-1-(sec-butyl)-2-oxo-1,2,3,5-tetrahydro-4H-pyrido[3,2-e][1,4]diazepin-4-yl)methyl)-N,N-dimethylpicolinamide;
6-((8-(4-((5-acetyl-2-((1S,2R)-2-fluorocyclopropane-1-carboxamido)pyridin-4-yl)amino)-3-methoxypyridin-2-yl)-1-((R)-sec-butyl)-2-oxo-1,2,3,5-tetrahydro-4H-pyrido[3,2-e][1,4]diazepin-4-yl)methyl)-N,N-dimethylpicolinamide;
5-((8-(3-((5-Acetyl-2-(cyclopropanecarboxamido)pyridin-4-yl)amino)-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylbicyclo[3.1.1]heptane-1-carboxamide;
6-((8-(4-((5-Acetyl-2-(cyclopropanecarboxamido)pyridin-4-yl)amino)-3-methoxypyridin-2-yl)-1-cyclopentyl-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-2-oxo-1-propyl-1,2,3,5-tetrahydro-4H-pyrido[3,2-e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-2-oxo-1-(pentan-3-yl)-1,2,3,5-tetrahydro-4H-pyrido[3,2-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-cyclohexyl-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-cyclohexyl-2-oxo-1,2,3,5-tetrahydro-4H-pyrido[3,2-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(tetrahydro-2H-pyran-4-yl)-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-5-fluoro-2-methoxyphenyl)-1-(2-hydroxy-2-methylpropyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-cyclopropyl-2-oxo-1,2,3,5-tetrahydro-4H-benzo[e][1,4]diazepin-4-yl)methyl)-N,N-dimethylpicolinamide;
3-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-2-methoxy-N,N-dimethylbenzamide;
3-((8-(3-((5-acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N,2-trimethylbenzamide;
6-(8-(3-((5-Acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylpicolinamide;
5-(8-(3-((5-Acetyl-2-(cyclopropanecarboxamido)pyridin-4-yl)amino)-2-methoxyphenyl)-1-(2,2-difluoroethyl)-2-oxo-2,3,4,5-tetrahydro-1H-pyrido[3,2-e][1,4]diazepine-4-carbonyl)-N,N-dimethylbicyclo[3.1.1]heptane-1-carboxamide;
6-((1-(4-(1H-Pyrazol-1-yl)phenyl)-8-(3-((5-acetyl-2-(cyclopropanecarboxamido)pyridin-4-yl)amino)-2-methoxyphenyl)-2-oxo-1,2,3,5-tetrahydro-4H-benzo[e][1,4]diazepin-4-yl)methyl)-N,N-dimethylpicolinamide;
6-((1-(6-(1H-Pyrazol-1-yl)pyridin-3-yl)-8-(3-((5-acetyl-2-(cyclopropanecarboxamido)pyridin-4-yl)amino)-2-methoxyphenyl)-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(6-methoxypyridin-3-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2-fluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3,4-difluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(quinolin-7-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(quinolin-6-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(5-fluoropyridin-2-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3-fluoropyridin-4-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2,4-difluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(4-cyanophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-phenyl-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3-chlorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(3-fluorophenyl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(6-methylpyridin-3-yl)-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-2-oxo-1-(quinolin-3-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(4-fluorophenyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(thiazol-5-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(pyridin-2-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-2-oxo-1-(pyridin-4-yl)-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(5-fluoropyridin-3-yl)-2-oxo-1,2,3,5-tetrahydro-4H-benzo[e][1,4]diazepin-4-yl)methyl)-N,N-dimethylpicolinamide;
6-((8-(3-((5-acetyl-2-(cyclopropanecarboxamido)pyridin-4-yl)amino)-2-methoxyphenyl)-1-(2-methoxypyridin-4-yl)-2-oxo-1,2,3,5-tetrahydro-4H-benzo[e][1,4]diazepin-4-yl)methyl)-N,N-dimethylpicolinamide;
N-(5-acetyl-4-((2-methoxy-3-(4-((6-(morpholine-4-carbonyl)pyridin-2-yl)methyl)-2-oxo-1-(pyridin-3-yl)-2,3,4,5-tetrahydro-1H-benzo[e][1,4]diazepin-8-yl)phenyl)amino)pyridin-2-yl)cyclopropanecarboxamide;
6-((8-(3-((5-acetyl-2-(cyclopropanecarboxamido)pyridin-4-yl)amino)-2-methoxyphenyl)-1-(methylsulfonyl)-2-oxo-1,2,3,5-tetrahydro-4H-benzo[e][1,4]diazepin-4-yl)methyl)-N,N-dimethylpicolinamide;
6-((8-(4-((5-acetyl-2-(cyclopropanecarboxamido)pyridin-4-yl)amino)-3-methoxypyridin-2-yl)-6-cyano-1-isopropyl-2-oxo-1,2,3,5-tetrahydro-4H-benzo[e][1,4]diazepin-4-yl)methyl)-N,N-dimethylpicolinamide;
(S)-6-((8-(4-((5-acetyl-2-(cyclopropanecarboxamido)pyridin-4-yl)amino)-3-methoxypyridin-2-yl)-1-(1,1-difluoropropan-2-yl)-2-oxo-1,2,3,5-tetrahydro-4H-pyrido[3,2-e][1,4]diazepin-4-yl)methyl)-N,N-dimethylpicolinamide;
(R)-6-((8-(4-((5-Acetyl-2-(cyclopropanecarboxamido)pyridin-4-yl)amino)-3-methoxypyridin-2-yl)-1-(1,1-difluoropropan-2-yl)-2-oxo-1,2,3,5-tetrahydro-4H-pyrido[3,2-e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-6-(ethylamino)-1-isopropyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-pyrido[2,3-e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-6-ethoxy-1-ethyl-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-6-ethoxy-1-ethyl-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-1-isopropyl-6-methoxy-2-oxo-1,2,3,5-tetrahydro-4H-benzo[e][1,4]diazepin-4-yl)methyl)-N,N-dimethylpicolinamide;
6-((8-(4-((5-Acetyl-2-(cyclopropanecarboxamido)pyridin-4-yl)amino)-3-methoxypyridin-2-yl)-6-chloro-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-ethyl-2-oxo-1,2,3,5-tetrahydro-4H-benzo[e][1,4]diazepin-4-yl)sulfonyl)-N,N-dimethylpicolinamide;
N-(5-Acetyl-4-((2-methoxy-3-(1-methyl-4-((6-(morpholine-4-carbonyl)pyridin-2-yl)sulfonyl)-2-oxo-2,3,4,5-tetrahydro-1H-benzo[e][1,4]diazepin-8-yl)phenyl)amino)pyridin-2-yl)cyclopropanecarboxamide;
6-((8-(3-((5-Acetyl-2-(cyclopropanecarboxamido)pyridin-4-yl)amino)-2-methoxyphenyl)-2-oxo-1-(pyridin-3-yl)-1,2,3,5-tetrahydro-4H-benzo[e][1,4]diazepin-4-yl)sulfonyl)-N,N-dimethylpicolinamide;
(R)-6-((8-(3-((5-Acetyl-2-(cyclopropanecarboxamido)pyridin-4-yl)amino)-2-methoxyphenyl)-1-ethyl-3-methyl-2-oxo-1,2,3,5-tetrahydro-4H-benzo[e][1,4]diazepin-4-yl)methyl)-N,N-dimethylpicolinamide;
(S)-6-((8-(3-((5-Acetyl-2-(cyclopropanecarboxamido)pyridin-4-yl)amino)-2-methoxyphenyl)-1-ethyl-3-methyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyridin-4-yl)amino)-2-methoxyphenyl)-1-ethyl-5-methyl-2-oxo-1,2,3,5-tetrahydro-4H-benzo[e][1,4]diazepin-4-yl)methyl)-N,N-dimethylpicolinamide;
6-((8-(3-((2-(Cyclopropanecarboxamido)-5-(2-fluoroacetyl)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((2-(Cyclopropanecarboxamido)-5-(2,2-difluoroacetyl)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-Acetyl-2-(cyclopropanecarboxamido)pyrimidin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((3-Acetyl-6-(cyclopropanecarboxamido)pyridazin-4-yl)amino)-2-methoxyphenyl)-1-methyl-2-oxo-1,2,3,5-tetrahydro-4H-benzo[e][1,4]diazepin-4-yl)methyl)-N,N-dimethylpicolinamide;
6-((8-(3-((2-(Cyclopropanecarboxamido)-5-oxo-5,6-dihydro-1,6-naphthyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-acetyl-2-(2-cyclopropylacetamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((8-(3-((5-acetyl-2-((1R,2R)-2-(trifluoromethyl)cyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(S)-6-((8-(3-((5-acetyl-2-(2,2-difluorocyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
(R)-6-((8-(3-((5-acetyl-2-(2,2-difluorocyclopropane-1-carboxamido)pyridin-4-yl)amino)-2-methoxyphenyl)-1-ethyl-2-oxo-1,2,3,5-tetrahydro-4H-benzo[e][1,4]diazepin-4-yl)methyl)-N,N-dimethylpicolinamide;
6-((8-(3-((5-acetyl-2-(2,2-dimethylcyclopropane-1-carboxamido)pyridin-4-yl)amino)-5-fluoro-2-methoxyphenyl)-1-(2,2-difluoroethyl)-2-oxo-1,2,3,5-tetrahydro-4H-pyrido[3,2-e][1,4]diazepin-4-yl)methyl)-N,N-dimethylpicolinamide;
6-((5-((1-((3′,6′-dihydroxy-3-oxo-3H-spiro[isobenzofuran-1,9′-xanthen]-5-yl)amino)-1-thioxo-5,8,11-trioxa-2-azatridecan-13-yl)carbamoyl)pyridin-2-yl)amino)-4-((3-(1-(1-((6-(dimethylcarbamoyl)pyridin-2-yl)methyl)azetidin-3-yl)-1H-pyrazol-4-yl)-2-methoxyphenyl)amino)-N-methylpyridazine-3-carboxamide;
or a pharmaceutically acceptable salt thereof.

44. The compound of claim 1 in the form of a pharmaceutically acceptable salt.

45. A pharmaceutical composition comprising a compound according to claim 1, or a pharmaceutically acceptable salt thereof, and a pharmaceutically acceptable excipient.

46-48. (canceled)

49. A method of treating a JAK2-mediated disorder in a patient in need thereof, comprising administering to the patient a compound of claim 1, or a pharmaceutically acceptable salt thereof, or a pharmaceutical composition comprising the compound.

50. The method of claim 49, wherein the JAK2-mediated disorder is a disorder mediated by a JAK2 containing a V617F mutation.

51. The method of claim 49, wherein the JAK2-mediated disorder is a proliferative disease.

52. The method of claim 49, wherein the proliferative disease displays overexpression or amplification of JAK2, or somatic mutation of JAK2.

53. The method of claim 49, wherein the JAK2-mediated disorder is myelofibrosis (MF), polycythemia Vera (PV), essential thrombocythemia (ET), acute megakaryocytic leukemia, T-cell acute lymphoblastic leukemia (T-ALL), B-cell acute lymphoblastic leukemia (B-ALL), acute myeloid leukemia (AML), Chronic Myelomonocytic Leukemia (CMML), T-cell large granular lymphocytic leukemia (T-LGL), T-cell prolymphocytic leukemia (T-PLL), or graft versus host disease (GVHD).

54. The method of claim 49, wherein the method comprises the steps of:
(i) identifying a subject in need of such treatment;
(ii) providing a disclosed compound, or a pharmaceutically acceptable salt thereof, and
(iii) administering said provided compound in a therapeutically effective amount to treat, suppress and/or prevent the disease state or condition in a subject in need of such treatment.
